(19) United States
(12) Patent Application Publication
(10) Pub. No.: US 2023/0151049 A1
(43) Pub. Date: May 18, 2023

(54) Compounds Useful in HIV Therapy

(71) Applicant: VIIV HEALTHCARE COMPANY, Wilmington, DE (US)

(72) Inventor: Namasimhulu B. NAIDU, Branford, CT (US)

(73) Assignee: VIIV HEALTHCARE COMPANY, Wilmington, DE (US)

(21) Appl. No.: 17/911,712

(22) PCT Filed: Mar. 18, 2021
(86) PCT No.: PCT/US2021/022880

§ 371 Date: Sep. 15, 2022

Related U.S. Application Data
(60) Provisional application No. 62/993,300, filed on Mar. 23, 2020.

(51) Int. Cl. C07H 21/02; A61K 45/06

(52) U.S. Cl. CPC C07H 21/02; A61K 45/06

(57) ABSTRACT

The invention relates to compounds of Formula (I), salts thereof, pharmaceutical compositions thereof, as well as methods of treating or preventing HIV in subjects.

### FIELD OF THE INVENTION

The present invention relates to compounds, pharmaceutical compositions, and methods of use thereof in connection with individuals infected with HIV. In particular, such methods of use encompass e.g., methods for treating HIV and methods of preventing HIV.

### BACKGROUND OF THE INVENTION

Human immunodeficiency virus type 1 (HIV-1) infection leads to the contraction of acquired immune deficiency disease (AIDS). The number of cases of HIV continues to rise, and currently an estimated over thirty-five million individuals worldwide suffer from HIV infection e.g., http://www.sciencedirect.com/science/article/pii/S235230181630087X? via %3Dihub

Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Hutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. Induction of the latent reservoir typically results in either direct death of the latently infected cell or killing of the induced cell by the immune system after the virus is made visible. As this is performed during ART, viral genomes produced are believed to not result in the infection of new cells and the size of the reservoir may decay.

HAART therapies are often complex because a combination of different drugs must be administered often daily to the patient to avoid the rapid emergence of drug-resistant HIV-1 variants. Despite the positive impact of HAART on patient survival, drug resistance can still occur.

Current guidelines recommend that therapy includes three fully active drugs. See e.g. https://aidsinfo.nih.gov/guidelines. Additionally, two drug combinations may be employed as therapeutic regimens. Typically, first-line therapies combine two to three drugs targeting the viral enzymes reverse transcriptase and integrase. It is believed that sustained successful treatment of HIV-1-infected patients with antiretroviral drugs employ the continued development of new and improved drugs that are effective against HIV strains that have formed resistance to approved drugs. For example, an individual on a regimen containing 3TC/FTC (lamivudine/emtricitabine) may select for the M184V mutation that reduces susceptibility to these drugs by >100 fold. See e g., https://hivdb.stanford.edu/dr-summary/resistance-notes/NRTI

Another way to potentially address preventing formation of mutations is to increase patient adherence to a drug regimen. One manner that may be employed to accomplish this is by reducing the dosing frequency. For parenteral administration, it is believed to be advantageous to have drug substances with high lipophilicity in order to reduce solubility and limit the release rate within interstitial fluid. However, most nucleoside reverse transcriptase inhibitors are hydrophilic thereby potentially limiting their use as long acting parenteral agents.

There remains a need for compounds which may address the shortcomings set forth above.

### SUMMARY OF THE INVENTION

In one aspect, the invention provides a compound of the formula (I):

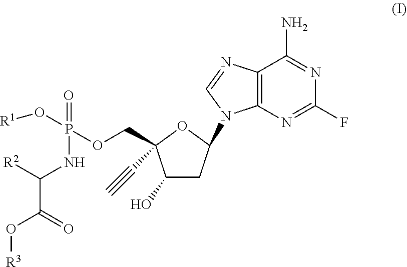

wherein:

R¹ is selected from the group consisting of (C₁-C₂₅) alkyl, (C₁-C₂₅) alkylaryl, (C₁-C₂₀) alkyl-CO₂R⁴ and aryl;

R² is selected from the group consisting of (C₁-C₁₀) alkyl; (C₁-C₁₀) alkylaryl and aryl;

R³ is selected from the group consisting of (C₁-C₂₅) alkyl, (C₁-C₂₅) alkylaryl and aryl;

R⁴ is selected from the group consisting of (C₁-C₂₅) alkyl, (C₁-C₂₅) alkylaryl and aryl; and

wherein each of R¹, R², R³, and R⁴ may be independently and optionally substituted by one or more (C₁-C₁₄) alkyl, Cl, F, oxo, or (C₁-C₆) alkoxy;

or a pharmaceutically acceptable salt thereof.

In another aspect, the invention provides pharmaceutical compositions comprising a compound of Formula (I) or a pharmaceutically acceptable salt thereof and an excipient.

In another aspect, the invention provides a method of treating an HIV infection in a patient comprising administering to the patient a compound of Formula (I), or a pharmaceutically acceptable salt thereof.

In another aspect, the invention provides a method of preventing an HIV infection in a subject at risk for developing an HIV infection, comprising administering to the subject a compound of Formula (I), or a pharmaceutically acceptable salt thereof.

In another aspect, there is provided a compound of Formula (I) or a pharmaceutically acceptable salt thereof for use in therapy.

In another aspect, there is provided a compound of Formula (I) or a pharmaceutically acceptable salt thereof for use in treating an HIV infection.

In another aspect, there is provided a compound of Formula (I) or a pharmaceutically acceptable salt thereof for use in preventing an HIV infection.

In another aspect, there is provided the use of a compound of Formula (I) or a pharmaceutically acceptable salt thereof in the manufacture of a medicament for treating an HIV infection.

In another aspect, there is provided the use of a compound of Formula (I) or a pharmaceutically acceptable salt thereof in the manufacture of a medicament for preventing an HIV infection.

These and other aspects are encompassed by the invention as set forth herein.

### BRIEF DESCRIPTION OF THE DRAWINGS

FIG. 1 represents the mean plasma concentration profiles of EfDA after subcutaneous dosing at 20 mpk in Wistar Han rats (N=3/time point.

FIG. 2 represents the mean plasma concentration profiles of EfDA after subcutaneous dosing at 20 mpk in Wistar Han rats (N=3/time point.

FIG. 3 represents the mean plasma concentration time profiles of Example 1A and EfDA after single IM injection of Example 1A at 20 mpk equivalent of EfDA in male Wistar Han rats (N=3/time point).

FIG. 4 represents the mean plasma concentration time profiles of Example 1B and EfDA after single IM injection of Example 1B at 62 mg/kg in male Wistar Han rats (N=3/time point).

FIG. 5 represents the mean plasma concentration time profiles of Example 2A and EfDA after single IM injection of Example 2A at 56 mg/kg in male Wistar Han rats (N=3/time point).

FIG. 6 represents the mean plasma concentration time profiles of Example 2B and EfDA after single IM injection of Example 2B at 20 mg/kg in male Wistar Han rats (N=3/time point).

FIG. 7 represents the mean plasma concentration time profiles of Example 4B and EfDA after single IM injection of Example 4B at 20 mg/kg in male Wistar Han rats (N=3/time point).

FIG. 8 represents the mean plasma concentration time profiles of Example 9B and EfDA after single IM injection of Example 9B at 20 mg/kg in male Wistar Han rats (N=3/time point).

FIG. 9 represents the mean plasma concentration time profiles of Example 11A and EfDA after single IM injection of Example 11A at 20 mg/kg in male Wistar Han rats (N=3/time point).

FIG. 10 represents the mean plasma concentration time profiles of Example 16A and EfDA after single IM injection of Example 16A at 20 mg/kg in male Wistar Han rats (N=3/time point).

FIG. 11 represents the mean plasma concentration time profiles of Example 18A and EfDA after single IM injection of Example 16A at 54 mg/kg in male Wistar Han rats (N=3/time point).

FIG. 12 represents the mean plasma concentration time profiles of Example 19A and EfDA after single IM injection of Example 19A at 55 mg/kg in male Wistar Han rats (N=3/time point).

FIG. 13 represents the mean plasma concentration time profiles of Example 20A and EfDA after single IM injection of Example 20A at 57 mg/kg in male Wistar Han rats (N=3/time point).

FIG. 14 represents the mean plasma concentration time profiles of Example 26A and EfDA after single IM injection of Example 26A at 63 mg/kg in male Wistar Han rats (N=3/time point).

FIG. 15 represents the mean plasma concentration time profiles of Example 37A and EfDA after single IM injection of Example 37A at 56 mg/kg in male Wistar Han rats (N=3/time point).

FIG. 16 represents the mean plasma concentration time profiles of Example 39A and EfDA after single IM injection of Example 39A at 58 mg/kg in male Wistar Han rats (N=3/time point).

FIG. 17 represents the mean plasma concentration time profiles of Example 40A and EfDA after single IM injection of Example 40A at 60 mg/kg in male Wistar Han rats (N=3/time point).

FIG. 18 represents the mean plasma concentration time profiles of Example 42B and EfDA after single IM injection of Example 42B at 64 mg/kg in male Wistar Han rats (N=3/time point).

FIG. 19 represents the individual plasma concentration time profiles of Example 43A and EfDA after single IM injection of Example 43A at 60 mg/kg in male Wistar Han rats (N=3/time point).

### DETAILED DESCRIPTION OF REPRESENTATIVE EMBODIMENTS

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

It is to be understood that the terminology used herein is for the purpose of describing particular embodiments only and is not intended to limit the scope of the present invention. In this specification and in the claims that follow, reference will be made to a number of terms that shall be defined to have the following meanings, unless otherwise noted herein by embodiments set forth.

The term “alkyl” represents a saturated, straight or branched hydrocarbon moiety having the specified number of carbon atoms, and many be monovalent or divalent. For example, the term “(C₁-C₂₅)alkyl” refers to an alkyl moiety containing e.g., from 1 to 25 carbon atoms. Exemplary alkyls include, but are not limited to, methyl, ethyl, n-propyl, isopropyl, n-butyl, isobutyl, s-butyl, t-butyl, pentyl, and hexyl. Other terms are encompassed by this definition, e.g., (C₁-C₂₀)alkyl, (C₁-C₁₀)alkyl, (C₁-C₅)alkyl and C₁alkyl.

The term “alkyl” may be used in combination with other substituent groups, such as e.g., “alkylaryl”, the term “alkyl” is intended to encompass a divalent straight or branched-chain hydrocarbon radical, wherein the point of attachment is through the alkyl moiety to the aryl group. Non-limiting examples of alkylaryl groups include (C₁-C₂₅)alkylaryl, (C₁-C₂₀)alkylaryl, (C₁-C₁₀)alkylaryl and (C₁)alkylaryl. Aryl in these embodiments is most preferably defined as C₆aryl.

The term “alkoxy” refers to a group containing an alkyl radical, defined hereinabove, attached through an oxygen linking atom. As one example, the term “(C₁-C₆)alkoxy” refers to a straight- or branched-chain hydrocarbon radical having at least 1 and up to 6 carbon atoms attached through an oxygen linking atom. Exemplary “(C₁-C₆)alkoxy” groups useful in the present invention include methoxy, ethoxy, n-propoxy, isopropoxy, n-butoxy, s-butoxy, isobutoxy, and t-butoxy. Other alkoxy ranges are contemplated within the scope of the invention.

The term “aryl” refers to optionally substituted monocyclic, fused bicyclic, or fused tricyclic groups having, for example, 6 to 14 carbon atoms and having at least one aromatic ring that complies with Hückers Rule. Examples of “aryl” groups are phenyl i.e., ((C₆)aryl), naphthyl, indenyl, dihydroindenyl, anthracenyl, phenanthrenyl, and the like.

The terms “halogen” and “halo” represent fluoro (F), chloro (Cl), bromo (Br), or iodo (I) substituents.”

The term “hydroxy” or “hydroxyl” is intended to mean the radical —OH.

The term “optionally” means that the subsequently described event(s) may or may not occur, and includes both event(s) that occur and event(s) that do not occur.

The term “oxo” refers to an “═O” group bonded to a carbon.

The term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. “Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002. For example, the pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The salt may precipitate from solution and be collected by filtration or may be recovered by evaporation of the solvent. The degree of ionization in the salt may vary from completely ionized to almost non-ionized. Lists of suitable salts are found in Remington's Pharmaceutical Sciences, 17th ed., Mack Publishing Company, Easton, Pa., 1985, p. 1418, the disclosure of which is hereby incorporated by reference only with regards to the lists of suitable salts.

The terms prevention or “preventing” a disease in a patient refers to the prophylactic administration of a drug to substantially diminish the likelihood or severity of a disorder or biological manifestation thereof, or to delay the onset of such disorder or biological manifestation thereof.

The term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

The terms “compound”, “compounds”, “chemical entity”, and “chemical entities” as used herein refers to a compound encompassed by the Formula (I) disclosed herein, any subgenus of these generic formulae, and any forms of the compounds within the generic formula, including the racemates, stereoisomers, and tautomers of the compound or compounds.

The term “heteroatom” means nitrogen, oxygen, or sulfur and includes any oxidized form of phosphorus, nitrogen, such as N(O) {N⁺—O⁻} and sulfur such as S(O) and S(O)₂, and the quaternized form of any basic nitrogen.

The terms“patient” or “subject” refers to mammals and includes humans and non-human mammals. Most preferably, a “patient” is construed to refer to humans.

In one aspect, there is provided a compound of the formula (I):

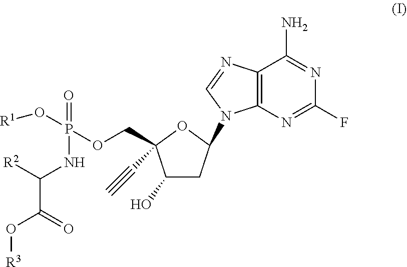

wherein:

R¹ is selected from the group consisting of (C₁-C₂₅) alkyl, (C₁-C₂₅) alkylaryl, (C₁-C₂₀) alkyl-CO₂R⁴ and aryl;

R² is selected from the group consisting of (C₁-C₁₀) alkyl; (C₁-C₁₀) alkylaryl and aryl;

R³ is selected from the group consisting of (C₁-C₂₅) alkyl, (C₁-C₂₅) alkylaryl and aryl; and

R⁴ is selected from the group consisting of (C₁-C₂₅) alkyl, (C₁-C₂₅) alkylaryl and aryl;

wherein each of R¹, R², R³, and R⁴ may be independently and optionally substituted by one or more (C₁-C₁₄) alkyl, Cl, F, oxo, or (C₁-C₆) alkoxy;

or a pharmaceutically acceptable salt thereof.

In one embodiment, R¹ is (C₆-C₁₄)aryl. More preferably, R¹ is C₆ aryl.

In one embodiment, R¹ is (C₁-C₂₀) alkyl-CO₂R₄. More preferably, R¹ is selected from (C₁-C₂₀) alkyl-CO₂R⁴, wherein R⁴ is (C₁-C₂₅) alkyl.

In one embodiment, R² is (C₁-C₁₀)alkyl(C₆-C₁₄)aryl. More preferably, R² is C₁ alkyl C₆aryl.

In one embodiment, R³ is (C₁-C₂₅)alkyl. More preferably, R³ is (C₅-C₂₅)alkyl. In one embodiment, R¹ is C₆ aryl, R² is C₁ alkyl or C₁ alkyl C₆aryl and R³ is (C₁-C₂₅)alkyl.

In one embodiment, R¹ is (C₁-C₂₅)alkyl-CO₂R⁴, R² is selected from C₁ alkyl or C₁ alkyl C₆ aryl, and R³ is (C₁-C₂₅)alkyl.

In one embodiment, R² is (C₁-C₁₀)alkyl. More preferably, R² is C₁₋₆ alkyl.

In additional to that set forth above, in further embodiments, the compounds of the present invention may be optionally substituted by one or more substituents as set forth below. For example, in one embodiment, each of R¹, R², R³, and R⁴ may be independently and optionally substituted by one or more (C₁-C₆) alkyl, Cl, F, oxo, or (C₁-C₆) alkoxy. Preferably, as an example, each of the aryl groups may be optionally substituted by one or more substituents from (C₁-C₅) alkyl, Cl, F, oxo, or (C₁-C₆) alkoxy.

In one embodiment, R² is (C₁-C₄) alkyl-C₆aryl. In another embodiment, R² is (C₁-C₄) alkyl-C₆aryl, wherein C₆ aryl is substituted by one or more substituents selected from Cl or F. In another embodiment, R² is (C₁-C₄) alkyl-C₆aryl, wherein C₆ aryl is substituted by one or more substituents which are F. In another embodiment, R² is (C₁) alkyl-C₆aryl, wherein C₆ aryl is substituted by two substituents which are F. In another embodiment, R² is:

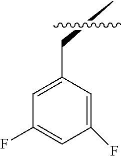

In one embodiment, R¹ is C₆ aryl, R² is is (C₁-C₄) alkyl-C₆ aryl, wherein C₆ aryl is substituted by one or more substituents which are each F and R³ is (C₁₀-C₂₅)alkyl. In another embodiment, R¹ is C₆ aryl, R² is is (C₁) alkyl-C₆ aryl, wherein C₆ aryl is substituted by two substituents which are each F, and R³ is (C₁₀-C₂₅)alkyl. In another embodiment, R¹ is C₆ aryl, R² is is (C₁) alkyl-C₆ aryl, wherein C₆ aryl is substituted by two substituents which are each F and is of the formula:

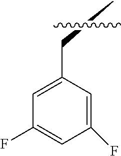

and R³ is (C₁₀-C₂₅)alkyl.

In one embodiment, R³ is (C₅-C₂₀) alkyl substituted by one or more substituents selected from Cl, F or both. In another embodiment, R³ is (C₅-C₂₀) alkyl substituted by one or more substituents selected from Cl, F or both. In another embodiment, R³ is (C₅-C₂₀) alkyl substituted by two or more substituents which are each F. In another embodiment, R³ is (C₅-C₁₅) alkyl, substituted by 5 to 15 substituents which are each F.

In another aspect of the present invention, the invention may encompass various individual compounds. As an example, such specific compounds may be selected from the group consisting of Table 1:

TABLE 1

| Example | Structure | Chemical Name |
|---|---|---|
| 1 | 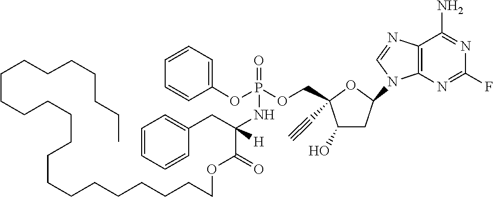 [SMILES: C#C[C@]1(COP(=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1O] | Docosyl ((((2R,3S,5R)-5-(6- amino-2-fluoro-9H-purin-9- yl)-2-ethynyl-3-hydroxy- tetrahydrofuran-2-yl)meth- oxy)(phenoxy)phosphoryl)- L-phenylalaninate |
| 2 | 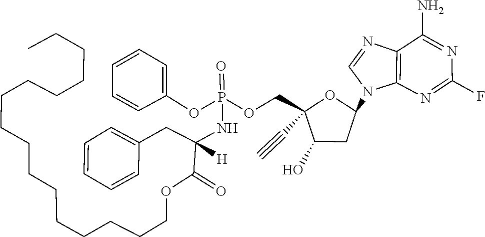 [SMILES: C#C[C@]1(COP(=O)(N[C@@H](Cc2ccccc2)C(=O)OCCCCCCCCCCCCCCCC)Oc2ccccc2)O[C@@H](n2cnc3c(N)nc(F)nc32)C[C@@H]1O] | Hexadecyl ((((2R,3S,5R)-5- (6-amino-2-fluoro-9H-purin- 9-yl)-2-ethynyl-3-hydroxy- tetrahydrofuran-2-yl)meth- oxy)(phenoxy)phosphoryl)- L-phenylalaninate |
| 3 | 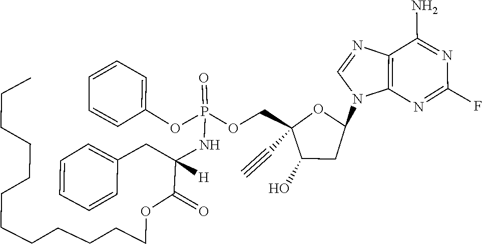 [SMILES: C#C[C@]1(COP(=O)(N[C@@H](Cc2ccccc2)C(=O)OCCCCCCCCCCCC)Oc2ccccc2)O[C@@H](n2cnc3c(N)nc(F)nc32)C[C@@H]1O] | Dodecyl ((((2R,3S,5R)-5-(6- amino-2-fluoro-9H-purin-9- yl)-2-ethynyl-3-hydroxy- tetrahydrofuran-2-yl)meth- oxy)(phenoxy)phosphoryl)- L-phenylalaninate |
| 4 | 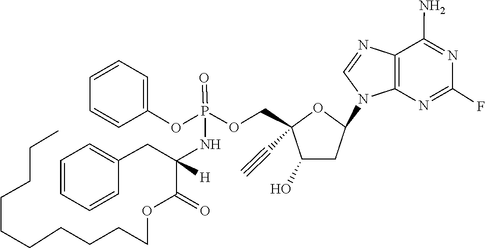 [SMILES: C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1O] | Decyl ((((2R,3S,5R)-5-(6- amino-2-fluoro-9H-purin-9- yl)-2-ethynyl-3-hydroxy- tetrahydrofuran-2-yl)meth- oxy)(phenoxy)phosphoryl)- L-phenylalaninate |
| 5 | 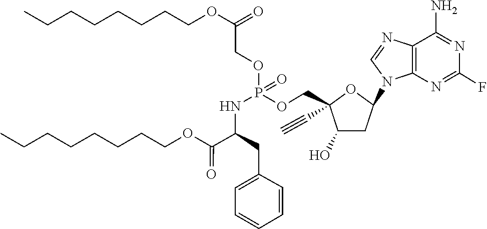 [SMILES: C#C[C@]1(COP(=O)(N[C@@H](Cc2ccccc2)C(=O)OCCCCCCCC)OCC(=O)OCCCCCCCC)O[C@@H](n2cnc3c(N)nc(F)nc32)C[C@@H]1O] | Octyl ((((2R,3S,5R)-5-(6- amino-2-fluoro-9H-purin-9- yl)-2-ethynyl-3-hydroxy- tetrahydrofuran-2-yl)meth- oxy)(2-(octyloxy)-2-oxo- ethoxy)phosphoryl)-L- phenylalaninate |
| 6 | 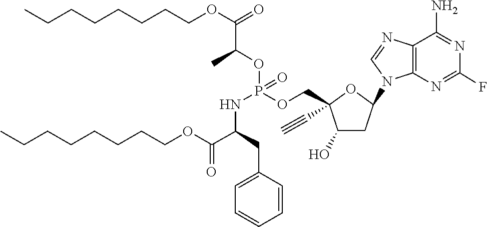 [SMILES: C#C[C@]1(COP(=O)(N[C@@H](Cc2ccccc2)C(=O)OCCCCCCCC)O[C@@H](C)C(=O)OCCCCCCCC)O[C@@H](n2cnc3c(N)nc(F)nc32)C[C@@H]1O] | Octyl ((((2R,3S,5R)-5-(6- amino-2-fluoro-9H-purin-9- yl)-2-ethynyl-3-hydroxy- tetrahydrofuran-2-yl)meth- oxy)(((S)-1-(octyloxy)-1- oxopropan-2-yl)oxy)phos- phoryl)-L-phenylalaninate |
| 7 | 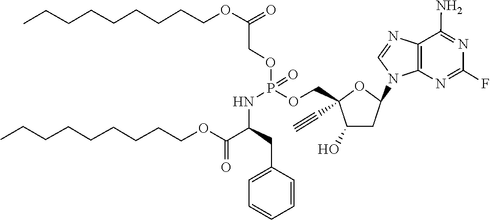 [SMILES: C#C[C@]1(COP(=O)(N[C@@H](Cc2ccccc2)C(=O)OCCCCCCCCC)OCC(=O)OCCCCCCCCC)O[C@@H](n2cnc3c(N)nc(F)nc32)C[C@@H]1O] | Nonyl ((((2R,3S,5R)-5-(6- amino-2-fluoro-9H-purin- 9-yl)-2-ethynyl-3-hydroxy- tetrahydrofuran-2-yl)meth- oxy)(2-(nonyloxy)-2-oxo- ethoxy)phosphoryl)-L- phenylalaninate |
| 8 | 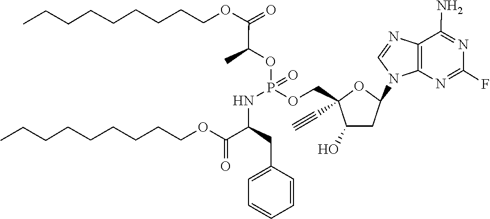 [SMILES: C#C[C@]1(COP(=O)(N[C@@H](Cc2ccccc2)C(=O)OCCCCCCCCC)O[C@@H](C)C(=O)OCCCCCCCCC)O[C@@H](n2cnc3c(N)nc(F)nc32)C[C@@H]1O] | Nonyl ((((2R,3S,5R)-5-(6- amino-2-fluoro-9H-purin- 9-yl)-2-ethynyl-3-hydroxy- tetrahydrofuran-2-yl)meth- oxy)(((S)-1-(nonyloxy)-1- oxopropan-2-yl)oxy)phos- phoryl)-L-phenylalaninate |
| 9 | 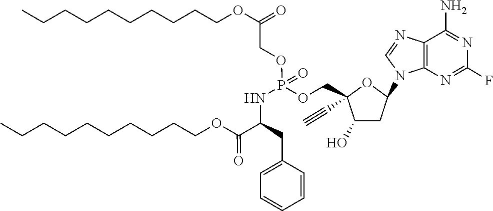 [SMILES: C#C[C@]1(COP(=O)(N[C@@H](Cc2ccccc2)C(=O)OCCCCCCCCCC)OCC(=O)OCCCCCCCCCC)O[C@@H](n2cnc3c(N)nc(F)nc32)C[C@@H]1O] | Decyl ((((2R,3S,5R)-5-(6- amino-2-fluoro-9H-purin- 9-yl)-2-ethynyl-3-hydroxy- tetrahydrofuran-2-yl)meth- oxy)(2-(decyloxy)-2-oxo- ethoxy)phosphoryl)-L- phenylalaninate |
| 10 | 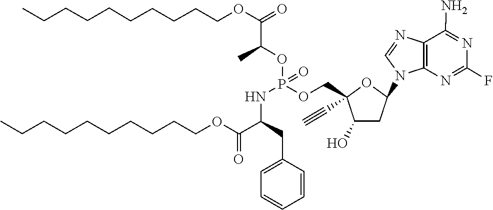 [SMILES: C#C[C@]1(COP(=O)(N[C@@H](Cc2ccccc2)C(=O)OCCCCCCCCCC)O[C@@H](C)C(=O)OCCCCCCCCCC)O[C@@H](n2cnc3c(N)nc(F)nc32)C[C@@H]1O] | Decyl ((((2R,3S,5R)-5-(6- amino-2-fluoro-9H-purin- 9-yl)-2-ethynyl-3-hydroxy- tetrahydrofuran-2-yl)meth- oxy)(((S)-1-(decyloxy)-1- oxopropan-2-yl)oxy)phos- phoryl)-L-phenylalaninate |
| 11 | 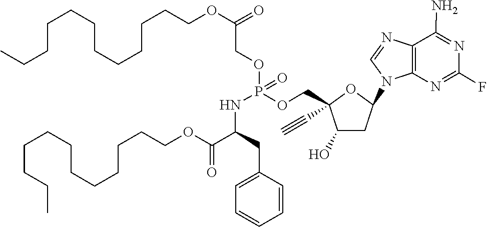 [SMILES: C#C[C@]1(COP(=O)(N[C@@H](Cc2ccccc2)C(=O)OCCCCCCCCCCCC)OCC(=O)OCCCCCCCCCCCC)O[C@@H](n2cnc3c(N)nc(F)nc32)C[C@@H]1O] | Dodecyl ((((2R,3S,5R)-5- (6-amino-2-fluoro-9H- purin-9-yl)-2-ethynyl-3- hydroxytetrahydrofuran-2- yl)methoxy)(2-(dodecyl- oxy)-2-oxoethoxy)phos- phoryl)-L-phenylalaninate |
| 12 | 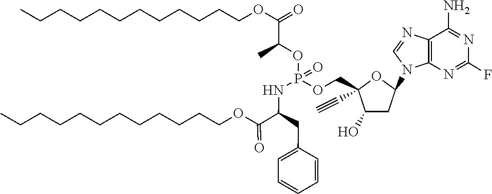 [SMILES: C#C[C@]1(COP(=O)(N[C@@H](Cc2ccccc2)C(=O)OCCCCCCCCCCCC)O[C@@H](C)C(=O)OCCCCCCCCCCCC)O[C@@H](n2cnc3c(N)nc(F)nc32)C[C@@H]1O] | Dodecyl ((((2R,3S,5R)-5- (6-amino-2-fluoro-9H- purin-9-yl)-2-ethynyl-3- hydroxytetrahydrofuran-2- yl)methoxy)(((S)-1- (dodecyloxy)-1-oxopropan- 2-yl)oxy)phosphoryl)-L- phenylalaninate |
| 13 | 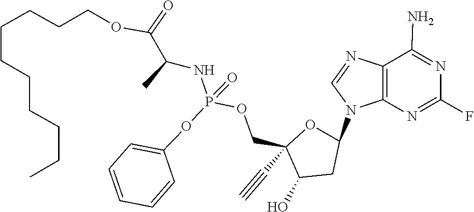 [SMILES: C#C[C@]1(COP(=O)(N[C@@H](C)C(=O)OCCCCCCCCCC)Oc2ccccc2)O[C@@H](n2cnc3c(N)nc(F)nc32)C[C@@H]1O] | Decyl ((((2R,3S,5R)-5- (6-amino-2-fluoro-9H- purin-9-yl)-2-ethynyl-3- hydroxytetrahydrofuran-2- yl)methoxy)(phenoxy)phos- phoryl)-L-alaninate |
| 14 | 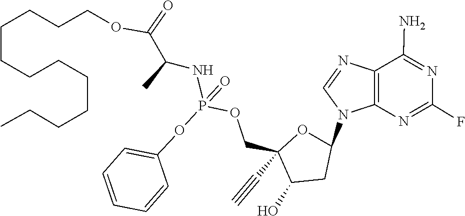 [SMILES: C#C[C@]1(COP(=O)(N[C@@H](C)C(=O)OCCCCCCCCCCCC)Oc2ccccc2)O[C@@H](n2cnc3c(N)nc(F)nc32)C[C@@H]1O] | Dodecyl ((((2R,3S,5R)-5- (6-amino-2-fluoro-9H- purin-9-yl)-2-ethynyl-3- hydroxytetrahydrofuran-2- yl)methoxy)(phenoxy)phos- phoryl)-L-alaninate |
| 15 | 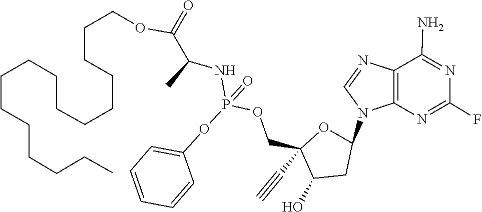 [SMILES: C#C[C@]1(COP(=O)(N[C@@H](C)C(=O)OCCCCCCCCCCCCCCCC)Oc2ccccc2)O[C@@H](n2cnc3c(N)nc(F)nc32)C[C@@H]1O] | Hexadecyl ((((2R,3S,5R)- (6-amino-2-fluoro-9H- purin-9-yl)-2-ethynyl-3- hydroxytetrahydrofuran-2- yl)methoxy)(phenoxy)- phosphoryl)-L-alaninate |
| 16 | 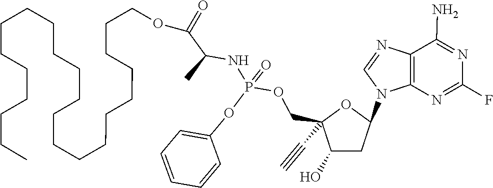 | Docosyl ((((2R,3S,5R)-5- (6-amino-2-fluoro-9H- purin-9-yl)-2-ethynyl-3- hydroxytetrahydrofuran- 2-yl)methoxy)(phenoxy)- phosphoryl)-L-alaninate |
| 17 | 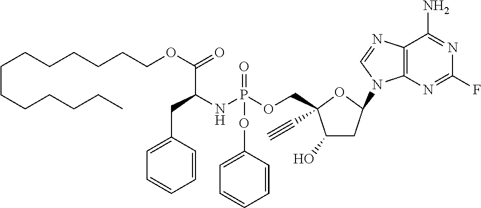 [SMILES: C#C[C@]1(COP(=O)(N[C@@H](Cc2ccccc2)C(=O)OCCCCCCCCCCCCC)Oc2ccccc2)O[C@@H](n2cnc3c(N)nc(F)nc32)C[C@@H]1O] | Tridecyl ((((2R,3S,5R)-5- (6-amino-2-fluoro-9H- purin-9-yl)-2-ethynyl-3- hydroxytetrahydrofuran- 2-yl)methoxy)(phenoxy)- phosphoryl)-L-phenyl- alaninate |
| 18 | 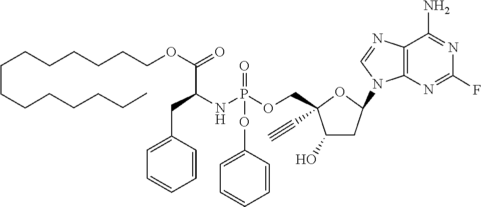 [SMILES: C#C[C@]1(COP(=O)(N[C@@H](Cc2ccccc2)C(=O)OCCCCCCCCCCCCCC)Oc2ccccc2)O[C@@H](n2cnc3c(N)nc(F)nc32)C[C@@H]1O] | Tetradecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 19 | 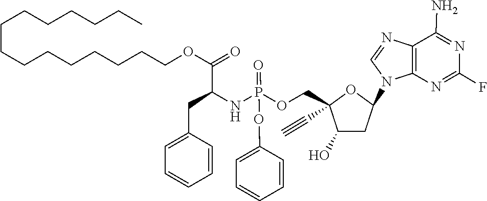 [SMILES: C#C[C@]1(COP(=O)(N[C@@H](Cc2ccccc2)C(=O)OCCCCCCCCCCCCCCC)Oc2ccccc2)O[C@@H](n2cnc3c(N)nc(F)nc32)C[C@@H]1O] | Pentadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 20 | 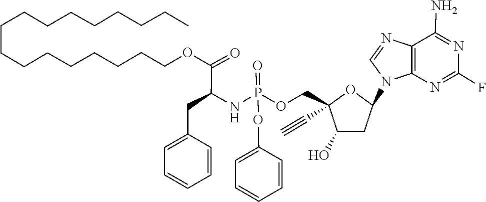 [SMILES: C#C[C@]1(COP(=O)(N[C@@H](Cc2ccccc2)C(=O)OCCCCCCCCCCCCCCCCC)Oc2ccccc2)O[C@@H](n2cnc3c(N)nc(F)nc32)C[C@@H]1O] | Heptadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 21 | 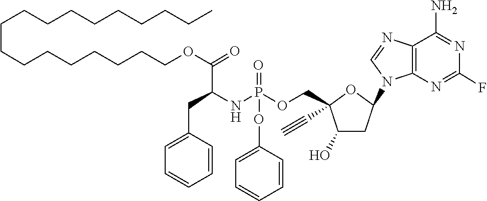 [SMILES: C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1O] | Octadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 22 | 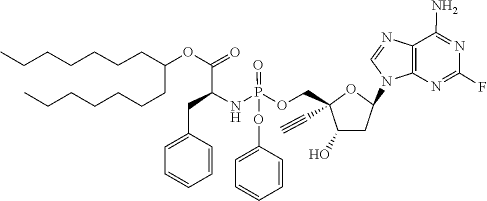 [SMILES: C#C[C@]1(COP(=O)(N[C@@H](Cc2ccccc2)C(=O)OC(CCCCCCC)CCCCCCC)Oc2ccccc2)O[C@@H](n2cnc3c(N)nc(F)nc32)C[C@@H]1O] | Pentadecan-8-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 23 | 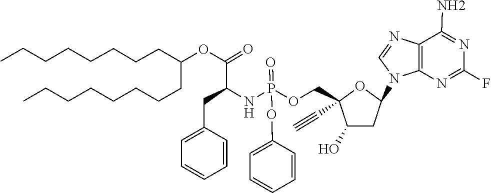 [SMILES: C#C[C@]1(COP(=O)(N[C@@H](Cc2ccccc2)C(=O)OC(CCCCCCCC)CCCCCCCC)Oc2ccccc2)O[C@@H](n2cnc3c(N)nc(F)nc32)C[C@@H]1O] | Heptadecan-9-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 24 | 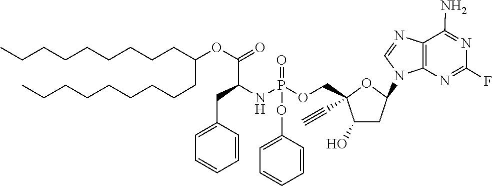 [SMILES: C#C[C@]1(COP(=O)(N[C@@H](Cc2ccccc2)C(=O)OC(CCCCCCCCC)CCCCCCCCC)Oc2ccccc2)O[C@@H](n2cnc3c(N)nc(F)nc32)C[C@@H]1O] | Nonadecan-10-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 25 | 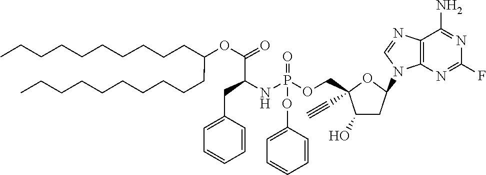 [SMILES: C#C[C@]1(COP(=O)(N[C@@H](Cc2ccccc2)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 26 | 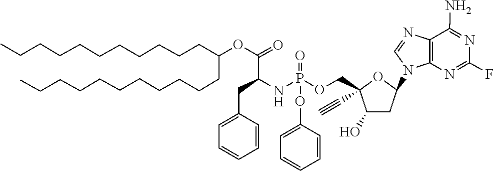 [SMILES: C#C[C@]1(COP(=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 27 | 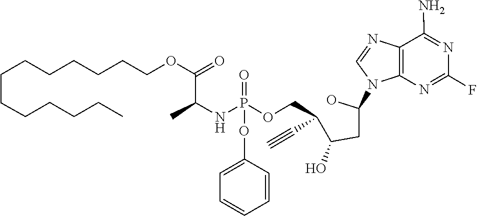 | Tridecyl ((((2R,3S,5R)- 5-(6-amino-2-fluoro-9H- purin-9-yl)-2-ethynyl-3- hydroxytetrahydrofuran- 2-yl)methoxy)(phenoxy)- phosphoryl)-L-alaninate |
| 28 | 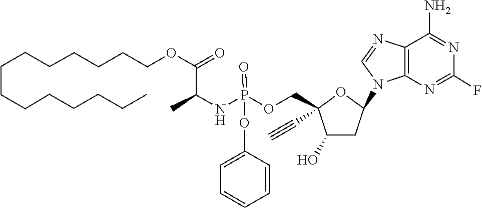 [SMILES: C#C[C@]1(COP(=O)(N[C@@H](C)C(=O)OCCCCCCCCCCCCCC)Oc2ccccc2)O[C@@H](n2cnc3c(N)nc(F)nc32)C[C@@H]1O] | Tetradecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 29 | 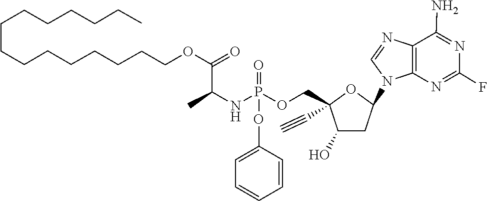 [SMILES: C#C[C@]1(COP(=O)(N[C@@H](C)C(=O)OCCCCCCCCCCCCCCC)Oc2ccccc2)O[C@@H](n2cnc3c(N)nc(F)nc32)C[C@@H]1O] | Pentadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 30 | 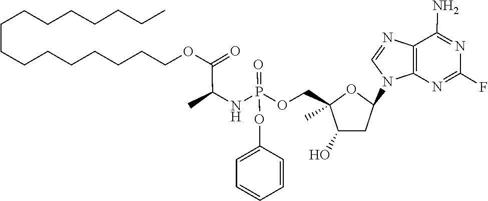 [SMILES: CCCCCCCCCCCCCCCCOC(=O)[C@H](C)NP(=O)(OC[C@@]1(C)O[C@@H](n2cnc3c(N)nc(F)nc32)C[C@@H]1O)Oc1ccccc1] | Hexadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 31 | 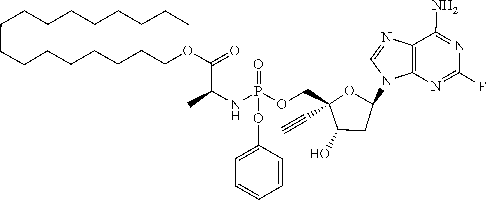 | Heptadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 32 | 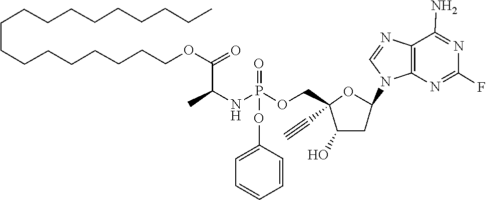 | Octadecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 33 | 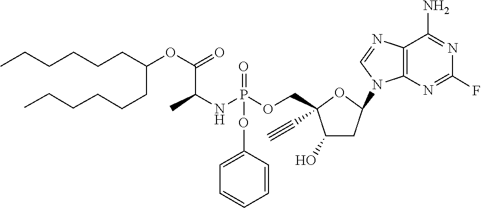 [SMILES: C#C[C@]1(COP(=O)(N[C@@H](C)C(=O)OC(CCCCCC)CCCCCC)Oc2ccccc2)O[C@@H](n2cnc3c(N)nc(F)nc32)C[C@@H]1O] | Tridecan-7-yl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 34 | 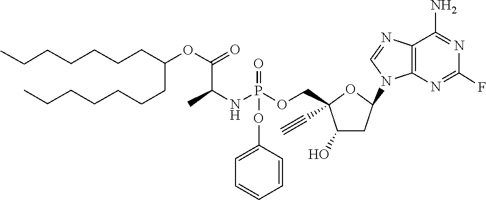 [SMILES: C#C[C@]1(COP(=O)(N[C@@H](C)C(=O)OC(CCCCCCC)CCCCCCC)Oc2ccccc2)O[C@@H](n2cnc3c(N)nc(F)nc32)C[C@@H]1O] | Pentadecan-8-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- alaninate |
| 35 | 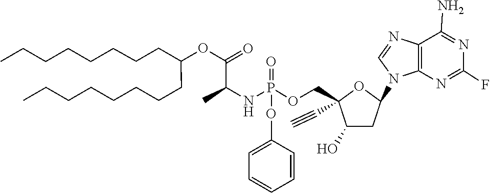 [SMILES: C#C[C@]1(COP(=O)(N[C@@H](C)C(=O)OC(CCCCCCCC)CCCCCCCC)Oc2ccccc2)O[C@@H](n2cnc3c(N)nc(F)nc32)C[C@@H]1O] | Heptadecan-9-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- alaninate |
| 36 | 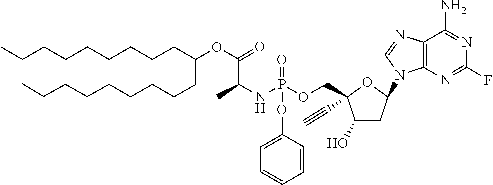 [SMILES: C#C[C@]1(COP(=O)(N[C@@H](C)C(=O)OC(CCCCCCCCC)CCCCCCCCC)Oc2ccccc2)O[C@@H](n2cnc3c(N)nc(F)nc32)C[C@@H]1O] | Nonadecan-10-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- alaninate |
| 37 | 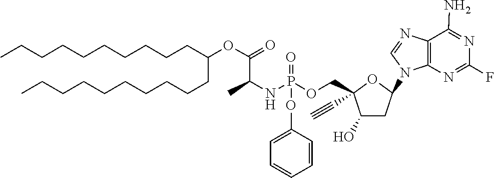 [SMILES: C#C[C@]1(COP(=O)(N[C@@H](C)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- alaninate |
| 38 | 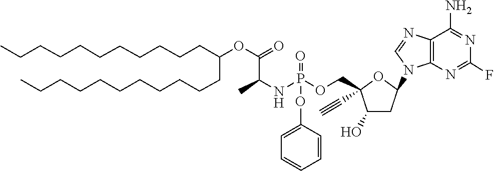 [SMILES: C#C[C@]1(COP(=O)(N[C@@H](C)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- alaninate |
| 39 | 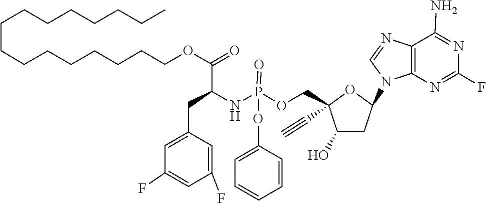 [SMILES: C#C[C@]1(COP(=O)(N[C@@H](Cc2cc(F)cc(F)c2)C(=O)OCCCCCCCCCCCCCCCC)Oc2ccccc2)O[C@@H](n2cnc3c(N)nc(F)nc32)C[C@@H]1O] | Hexadecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 40 | 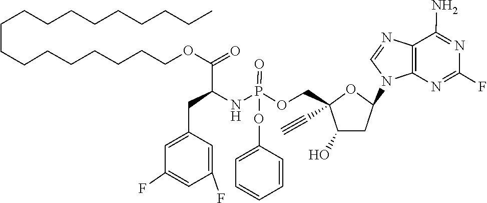 [SMILES: C#C[C@]1(COP(=O)(N[C@@H](Cc2cc(F)cc(F)c2)C(=O)OCCCCCCCCCCCCCCCCCC)Oc2ccccc2)O[C@@H](n2cnc3c(N)nc(F)nc32)C[C@@H]1O] | Octadecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 41 | 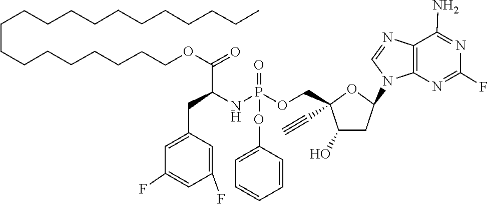 [SMILES: C#C[C@]1(COP(=O)(N[C@@H](Cc2cc(F)cc(F)c2)C(=O)OCCCCCCCCCCCCCCCCCCCC)Oc2ccccc2)O[C@@H](n2cnc3c(N)nc(F)nc32)C[C@@H]1O] | Icosyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 42 | 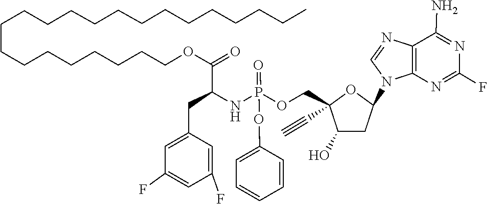 [SMILES: C#C[C@]1(COP(=O)(N[C@@H](Cc2cc(F)cc(F)c2)C(=O)OCCCCCCCCCCCCCCCCCCCCCC)Oc2ccccc2)O[C@@H](n2cnc3c(N)nc(F)nc32)C[C@@H]1O] | Docosyl (2S)-2- (((((2R,3S,5R)-5-(6-amino- 2-fluoro-9H-purin-9-yl)- 2-ethynyl-3-hydroxytetra- hydrofuran-2-yl)methoxy)- (phenoxy)phosphoryl)- amino)-3-(3,5-difluoro- phenyl)propanoate |
| 43 | 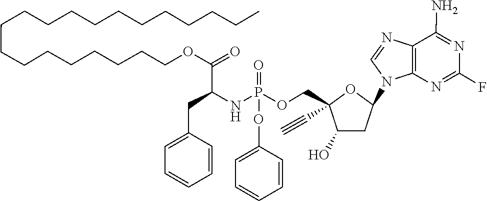 [SMILES: C#C[C@]1(COP(=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1O] | Icosyl ((((2R,3S,5R)-5-(6- amino-2-fluoro-9H-purin- 9-yl)-2-ethynyl-3-hydroxy- tetrahydrofuran-2-yl)meth- oxy)(phenoxy)phosphoryl)- L-phenylalaninate |
| 44 | 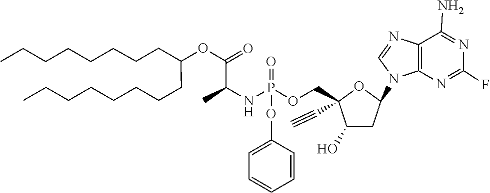 [SMILES: C#C[C@]1(COP(=O)(N[C@@H](C)C(=O)OC(CCCCCCCC)CCCCCCCC)Oc2ccccc2)O[C@@H](n2cnc3c(N)nc(F)nc32)C[C@@H]1O] | 12,12,13,13,14,14,15,15,15- Nonafluoropentadecyl ((((2R,3S,5R)-5-(6-amino- 2-fluoro-9H-purin-9-yl)- 2-ethynyl-3-hydroxytetra- hydrofuran-2-yl)methoxy)- (phenoxy)phosphoryl)- L-phenylalaninate |
| 45 | 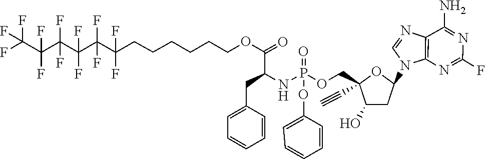 [SMILES: C#C[C@]1(COP(=O)(N[C@@H](Cc2ccccc2)C(=O)OCCCCCCC(F)(F)C(F)(F)C(F)(F)C(F)(F)C(F)(F)C(F)(F)F)Oc2ccccc2)O[C@@H](n2cnc3c(N)nc(F)nc32)C[C@@H]1O] | 7,7,8,8,9,9,10,10,11,11, 12,12,12-Tridecafluoro- dodecyl ((((2R,3S,5R)-5-(6- amino-2-fluoro-9H-purin-9- yl)-2-ethynyl-3-hydroxy- tetrahydrofuran-2-yl)meth- oxy)(phenoxy)phosphoryl)- L-phenylalaninate |
| 46 | 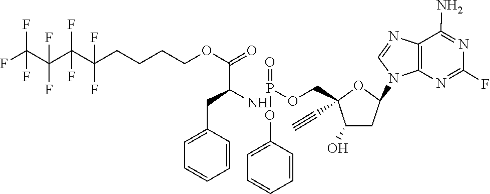 | 5,5,6,6,7,7,8,8,8-Nona- fluorooctyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 47 | 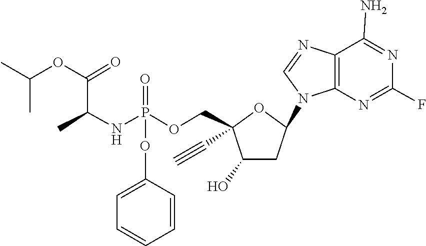 [SMILES: C#C[C@]1(COP(=O)(N[C@@H](C)C(=O)OC(C)C)Oc2ccccc2)O[C@@H](n2cnc3c(N)nc(F)nc32)C[C@@H]1O] | Isopropyl ((((2R,3S,5R)-5- (6-amino-2-fluoro-9H- purin-9-yl)-2-ethynyl-3- hydroxytetrahydrofuran-2- yl)methoxy)(phenoxy)phos- phoryl)-L-alaninate |
| 48 | 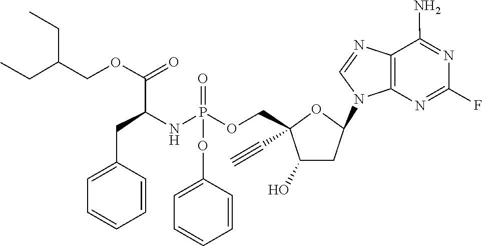 [SMILES: C#C[C@]1(COP(=O)(N[C@@H](Cc2ccccc2)C(=O)OCC(CC)CC)Oc2ccccc2)O[C@@H](n2cnc3c(N)nc(F)nc32)C[C@@H]1O] | 2-Ethylbutyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 49 | 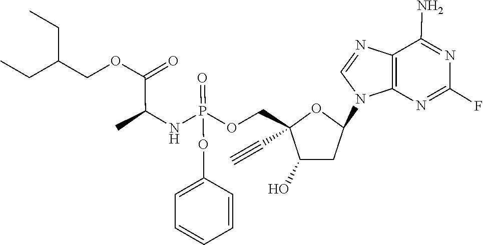 [SMILES: C#C[C@]1(COP(=O)(N[C@@H](C)C(=O)OCC(CC)CC)Oc2ccccc2)O[C@@H](n2cnc3c(N)nc(F)nc32)C[C@@H]1O] | 2-Ethylbutyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-alaninate |
| 50 | 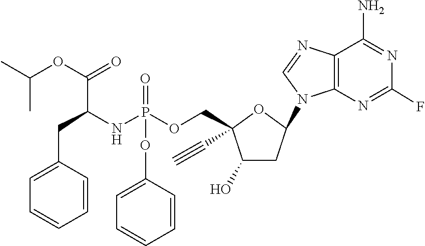 [SMILES: C#C[C@]1(COP(=O)(N[C@@H](Cc2ccccc2)C(=O)OC(C)C)Oc2ccccc2)O[C@@H](n2cnc3c(N)nc(F)nc32)C[C@@H]1O] | Isopropyl ((((2R,3S,5R)-5- (6-amino-2-fluoro-9H-purin- 9-yl)-2-ethynyl-3-hydroxy- tetrahydrofuran-2-yl)meth- oxy)(phenoxy)phosphoryl)- L-phenylalaninate |
| 51 | 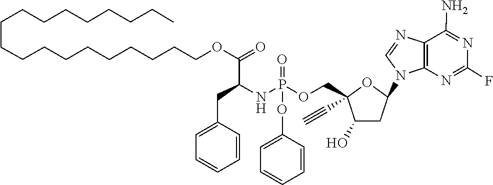 [SMILES: C#C[C@]1(COP(=O)(N[C@@H](Cc2ccccc2)C(=O)OCCCCCCCCCCCCCCCCCCC)Oc2ccccc2)O[C@@H](n2cnc3c(N)nc(F)nc32)C[C@@H]1O] | Nonadecyl ((((2R,3S,5R)-5- (6-amino-2-fluoro-9H-purin- 9-yl)-2-ethynyl-3-hydroxy- tetrahydrofuran-2-yl)meth- oxy)(phenoxy)phosphoryl)- L-phenylalaninate |
| 52 | 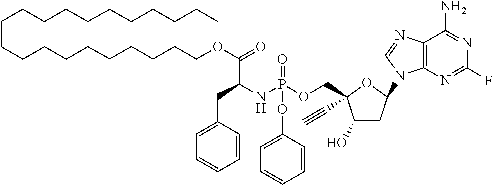 [SMILES: C#C[C@]1(COP(=O)(N[C@@H](Cc2ccccc2)C(=O)OCCCCCCCCCCCCCCCCCCCCC)Oc2ccccc2)O[C@@H](n2cnc3c(N)nc(F)nc32)C[C@@H]1O] | Henicosyl ((((2R,3S,5R)-5- (6-amino-2-fluoro-9H-purin- 9-yl)-2-ethynyl-3-hydroxy- tetrahydrofuran-2-yl)meth- oxy)(phenoxy)phosphoryl)- L-phenylalaninate |
| 53 | 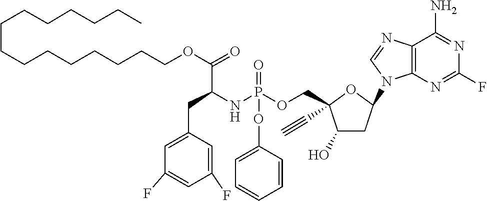 [SMILES: C#C[C@]1(COP(=O)(N[C@@H](Cc2cc(F)cc(F)c2)C(=O)OCCCCCCCCCCCCCCC)Oc2ccccc2)O[C@@H](n2cnc3c(N)nc(F)nc32)C[C@@H]1O] | Pentadecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 54 | 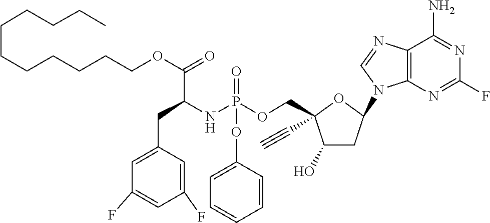 [SMILES: C#C[C@]1(COP(=O)(N[C@@H](Cc2cc(F)cc(F)c2)C(=O)OCCCCCCCCCCC)Oc2ccccc2)O[C@@H](n2cnc3c(N)nc(F)nc32)C[C@@H]1O] | Undecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 55 | 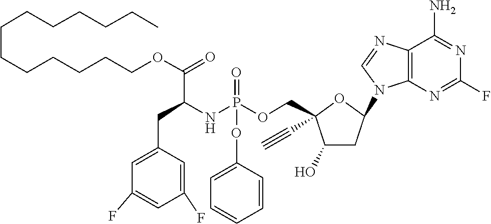 [SMILES: C#C[C@]1(COP(=O)(N[C@@H](Cc2cc(F)cc(F)c2)C(=O)OCCCCCCCCCCCCC)Oc2ccccc2)O[C@@H](n2cnc3c(N)nc(F)nc32)C[C@@H]1O] | Tridecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 56 | 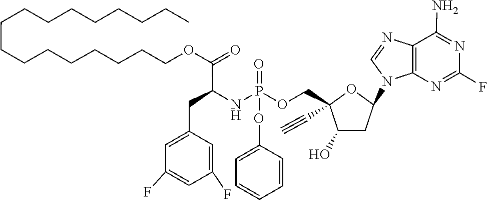 [SMILES: C#C[C@]1(COP(=O)(N[C@@H](Cc2cc(F)cc(F)c2)C(=O)OCCCCCCCCCCCCCCCCC)Oc2ccccc2)O[C@@H](n2cnc3c(N)nc(F)nc32)C[C@@H]1O] | Heptadecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 57 | 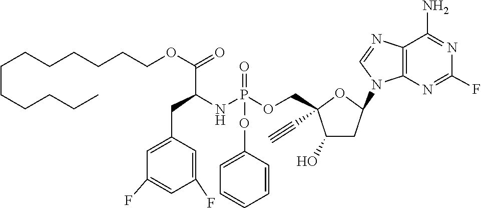 [SMILES: C#C[C@]1(COP(=O)(N[C@@H](Cc2cc(F)cc(F)c2)C(=O)OCCCCCCCCCCCC)Oc2ccccc2)O[C@@H](n2cnc3c(N)nc(F)nc32)C[C@@H]1O] | Dodecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 58 | 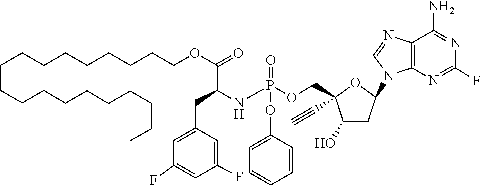 [SMILES: C#C[C@]1(COP(=O)(N[C@@H](Cc2cc(F)cc(F)c2)C(=O)OCCCCCCCCCCCCCCCCCCC)Oc2ccccc2)O[C@@H](n2cnc3c(N)nc(F)nc32)C[C@@H]1O] | Nonadecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 59 | 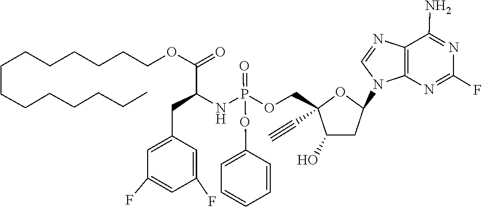 [SMILES: C#C[C@]1(COP(=O)(N[C@@H](Cc2cc(F)cc(F)c2)C(=O)OCCCCCCCCCCCCCC)Oc2ccccc2)O[C@@H](n2cnc3c(N)nc(F)nc32)C[C@@H]1O] | Tetradecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 60 | 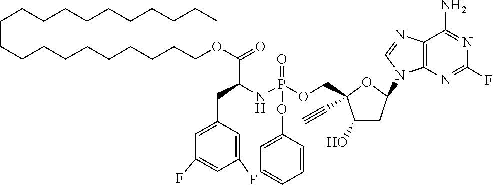 [SMILES: C#C[C@]1(COP(=O)(N[C@@H](Cc2cc(F)cc(F)c2)C(=O)OCCCCCCCCCCCCCCCCCCCCC)Oc2ccccc2)O[C@@H](n2cnc3c(N)nc(F)nc32)C[C@@H]1O] | Henicosyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluoro- phenyl)propanoate |
| 61 | 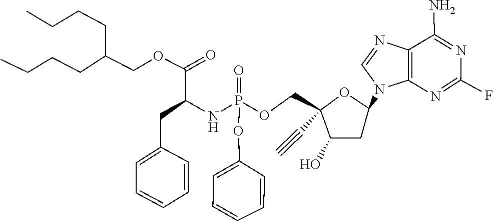 [SMILES: C#C[C@]1(COP(=O)(N[C@@H](Cc2ccccc2)C(=O)OCC(CCCC)CCCC)Oc2ccccc2)O[C@@H](n2cnc3c(N)nc(F)nc32)C[C@@H]1O] | 2-Butylhexyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 62 | 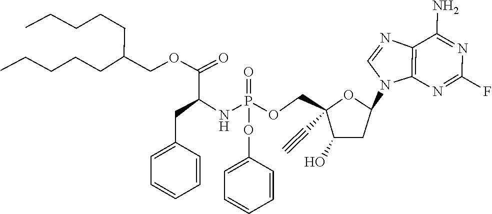 [SMILES: C#C[C@]1(COP(=O)(N[C@@H](Cc2ccccc2)C(=O)OCC(CCCCC)CCCCC)Oc2ccccc2)O[C@@H](n2cnc3c(N)nc(F)nc32)C[C@@H]1O] | 2-Pentylheptyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 63 | 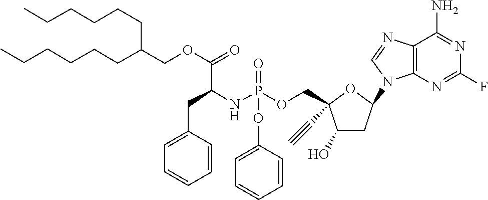 [SMILES: C#C[C@]1(COP(=O)(N[C@@H](Cc2ccccc2)C(=O)OCC(CCCCCC)CCCCCC)Oc2ccccc2)O[C@@H](n2cnc3c(N)nc(F)nc32)C[C@@H]1O] | 2-Hexyloctyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 64 | 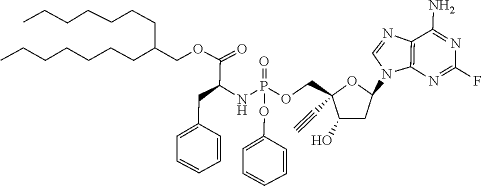 [SMILES: C#C[C@]1(COP(=O)(N[C@@H](Cc2ccccc2)C(=O)OCC(CCCCCCC)CCCCCCC)Oc2ccccc2)O[C@@H](n2cnc3c(N)nc(F)nc32)C[C@@H]1O] | 2-Hexyloctyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 65 | 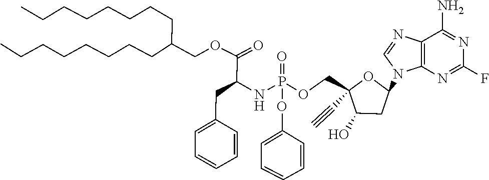 [SMILES: C#C[C@]1(COP(=O)(N[C@@H](Cc2ccccc2)C(=O)OCC(CCCCCCCC)CCCCCCCC)Oc2ccccc2)O[C@@H](n2cnc3c(N)nc(F)nc32)C[C@@H]1O] | 2-Octyldecyl ((((2R,3S,5R)- 5-(6-amino-2-fluoro-9H- purin-9-yl)-2-ethynyl-3- hydroxytetrahydrofuran-2- yl)methoxy)(phenoxy)phos- phoryl)-L-phenylalaninate |
| 66 | 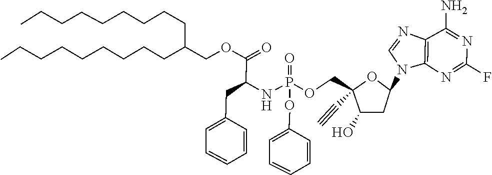 [SMILES: C#C[C@]1(COP(=O)(N[C@@H](Cc2ccccc2)C(=O)OCC(CCCCCCCCC)CCCCCCCCC)Oc2ccccc2)O[C@@H](n2cnc3c(N)nc(F)nc32)C[C@@H]1O] | 2-Nonylundecyl ((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)-L- phenylalaninate |
| 67 | 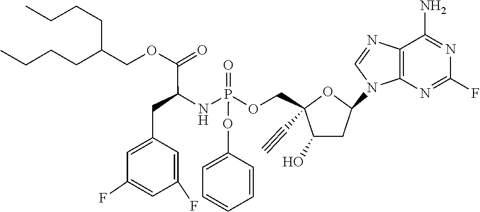 [SMILES: C#C[C@]1(COP(=O)(N[C@@H](Cc2cc(F)cc(F)c2)C(=O)OCC(CCCC)CCCC)Oc2ccccc2)O[C@@H](n2cnc3c(N)nc(F)nc32)C[C@@H]1O] | 2-Butylhexyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |
| 68 | 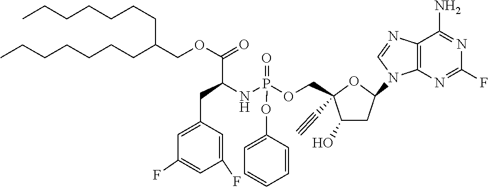 [SMILES: C#C[C@]1(COP(=O)(N[C@@H](Cc2cc(F)cc(F)c2)C(=O)OCC(CCCCCCC)CCCCCCC)Oc2ccccc2)O[C@@H](n2cnc3c(N)nc(F)nc32)C[C@@H]1O] | 2-Heptylnonyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |
| 69 | 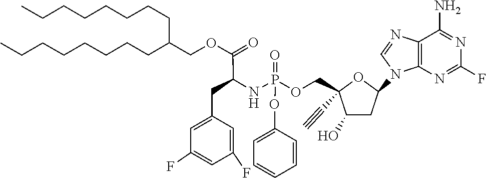 [SMILES: C#C[C@]1(COP(=O)(N[C@@H](Cc2cc(F)cc(F)c2)C(=O)OCC(CCCCCCCC)CCCCCCCC)Oc2ccccc2)O[C@@H](n2cnc3c(N)nc(F)nc32)C[C@@H]1O] | 2-Octyldecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |
| 70 | 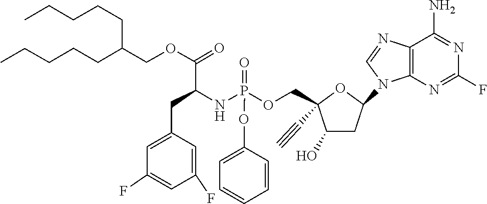 [SMILES: C#C[C@]1(COP(=O)(N[C@@H](Cc2cc(F)cc(F)c2)C(=O)OCC(CCCCC)CCCCC)Oc2ccccc2)O[C@@H](n2cnc3c(N)nc(F)nc32)C[C@@H]1O] | 2-Pentylheptyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |
| 71 | 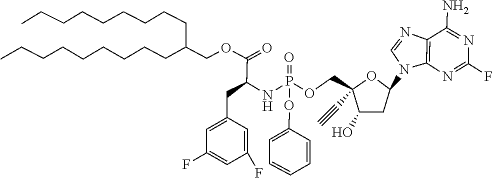 [SMILES: C#C[C@]1(COP(=O)(N[C@@H](Cc2cc(F)cc(F)c2)C(=O)OCC(CCCCCCCCC)CCCCCCCCC)Oc2ccccc2)O[C@@H](n2cnc3c(N)nc(F)nc32)C[C@@H]1O] | 2-Nonylundecyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |
| 72 | 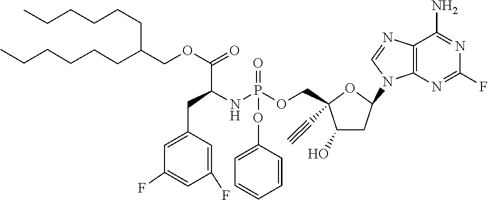 [SMILES: C#C[C@]1(COP(=O)(N[C@@H](Cc2cc(F)cc(F)c2)C(=O)OCC(CCCCCC)CCCCCC)Oc2ccccc2)O[C@@H](n2cnc3c(N)nc(F)nc32)C[C@@H]1O] | 2-Hexyloctyl (2S)-2- (((((2R,3S,5R)-5-(6-amino- 2-fluoro-9H-purin-9-yl)-2- ethynyl-3-hydroxytetra- hydrofuran-2-yl)methoxy)- (phenoxy)phosphoryl)- amino)-3-(3,5-difluorophen- yl)propanoate |

and pharmaceutically acceptable salts thereof.

In one embodiment, the present invention encompasses each individual compound listed in the above Table 1, or a pharmaceutically acceptable salt thereof.

In various embodiments, prodrugs of any of the compounds of formula (I) set forth herein are also within the scope of the present invention.

In accordance with one embodiment of the present invention, there is provided a pharmaceutical composition comprising a compound of Formulas (I) or a pharmaceutically acceptable salt thereof, and a pharmaceutically acceptable excipient. In a further embodiment, the compound is present in amorphous form. In a further embodiment, the compound is present in crystalline form. In a further embodiment, the pharmaceutical composition is in a tablet form. In a further embodiment, the pharmaceutical composition is in parenteral form. In a further embodiment, the compound is present as a spray dried dispersion.

In accordance with one embodiment of the present invention, there is provided a method of treating an HIV infection in a subject comprising administering to the subject a compound of Formulas (I) or a pharmaceutically acceptable salt thereof.

In accordance with one embodiment of the present invention, there is provided a method of treating an HIV infection in a subject comprising administering to the subject a pharmaceutical composition as described herein.

In accordance with one embodiment of the present invention, there is provided a method of preventing an HIV infection in a subject at risk for developing an HIV infection, comprising administering to the subject a compound of Formulas (I) or a pharmaceutically acceptable salt thereof.

In accordance with one embodiment of the present invention, there is provided the use of a compound of Formula (I) in the manufacture of a medicament for treating an HIV infection.

In accordance with one embodiment of the present invention, there is provided the use of a compound of Formula (I) in the manufacture of a medicament for preventing an HIV infection.

In accordance with one embodiment of the present invention, there is provided a compound according to Formula (I) for use in treating an HIV infection.

In accordance with one embodiment of the present invention, there is provided a compound according to Formula (I) for use in preventing an HIV infection.

In accordance with one embodiment of the present invention, there is provided a method of preventing an HIV infection in a subject at risk for developing an HIV infection, comprising administering to the subject a pharmaceutical composition as described herein.

Furthermore, the compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the present invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

In another embodiment of the invention, there is provided a compound of Formula (I) wherein the compound or salt of the compound is used in the manufacture of a medicament for use in the treatment of an HIV infection in a human.

In another embodiment of the invention, there is provided a compound of Formula (I) wherein the compound or salt of the compound is used in the manufacture of a medicament for use in the prevention of an HIV infection in a human.

In one embodiment, the pharmaceutical formulation containing a compound of Formula (I) or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formula (I) further comprise administration of one or more additional pharmaceutical agents active against HIV.

In such embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, bictegravir, vicriviroc (Sch-C), Sch-D, TAK779, maraviroc, TAK449, didanosine, tenofovir, lopinavir, and darunavir.

As such, the compounds of the present invention of Formulas (I) and any other pharmaceutically active agent(s) may be administered together or separately and, when administered separately, administration may occur simultaneously or sequentially, in any order. The amounts of the compounds of Formula (I) of the present invention and the other pharmaceutically active agent(s) and the relative timings of administration will be selected in order to achieve the desired combined therapeutic effect. The administration in combination of a compound of the present invention of Formula (I) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula (I) or salts thereof and the other pharmaceutically active agent(s) and the relative timings of administration will be selected in order to achieve the desired combined therapeutic effect.

In addition, the compounds of the present invention of Formula (I) may be used in combination with one or more other agents that may be useful in the prevention or treatment of HIV. Examples of such agents include:

Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, doravirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068 (Fostemsavir), BMS-626529 (Temsavir), 5-Helix and similar agents;
Integrase inhibitors such as raltegravir, elvitegravir, dolutegravir, bictegravir, cabotegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.
CAPSID inhibitors such GS-6207, and similar agents.

Further examples where the compounds of the present invention may be used in combination with one or more agents useful in the prevention or treatment of HIV are found in Table 2.

TABLE 2

| | | | |
|---|---|---|---|
| FDA | Brand | | |
| Approval | Name | Generic Name | Manufacturer |

Nucleoside Reverse Transcriptase Inhibitors (NRTIs)

| | | | |
|---|---|---|---|
| 1987 | Retrovir | zidovudine, azidothymidine, | GlaxoSmithKline |
| | | AZT, ZDV | |
| 1991 | Videx | didanosine, dideoxyinosine, | Bristol-Myers |
| | | ddI | Squibb |
| 1992 | Hivid | zalcitabine, | Roche |
| | | dideoxycytidine, ddC | Pharmaceuticals |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + zidovudine | GlaxoSmithKline |
| 1998 | Ziagen | abacavir sulfate, ABC | GlaxoSmithKline |
| 2000 | Trizivir | abacavir + lamivudine + | GlaxoSmithKline |
| | | zidovudine | |
| 2000 | Videx EC | enteric coated didanosine, | Bristol-Myers |
| | | ddI EC | Squibb |
| 2001 | Viread | tenofovir disoproxil | Gilead Sciences |
| | | fumarate, TDF | |
| 2003 | Emtriva | emtricitabine, FTC | Gilead Sciences |
| 2004 | Epzicom | abacavir + lamivudine | GlaxoSmithKline |
| 2004 | Truvada | emtricitabine + tenofovir | Gilead Sciences |
| | | disoproxil fumarate | |

Non-Nucleosides Reverse Transcriptase Inhibitors (NNRTIs)

| | | | |
|---|---|---|---|
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |
| 2011 | Edurant | Rilpivirine | Tibotec |
| | | | Therapeutics |
| 2018 | Pifeltro | Doravirine | Merck |

Protease Inhibitors (PIs)

| | | | |
|---|---|---|---|
| 1995 | Invirase | Saquinavir mesylate, SQV | Roche |
| | | | Pharmaceuticals |
| 1996 | Norvir | ritonavir, RTV | Abbott |
| | | | Laboratories |
| 1996 | Crixivan | indinavir, IDV | Merck |
| 1997 | Viracept | Nelfinavir mesylate, NFV | Pfizer |
| 1997 | Fortovase | saquinavir (no longer | Roche |
| | | marketed) | Pharmaceuticals |
| 1999 | Agenerase | amprenavir, APV | GlaxoSmithKline |
| 2000 | Kaletra | lopinavir + ritonavir, | Abbott |
| | | LPV/RTV | Laboratories |
| 2003 | Reyataz | atazanavir sulfate, ATV | Bristol-Myers |
| | | | Squibb |
| 2003 | Lexiva | Fosamprenavir calcium, | GlaxoSmithKline |
| | | FOS-APV | |
| 2005 | Aptivus | tripranavir, TPV | Boehringer |
| | | | Ingelheim |
| 2006 | Prezista | Darunavir | Tibotec |
| | | | Therapeutics |

Fusion Inhibitors

| | | | |
|---|---|---|---|
| 2003 | Fuzeon | Enfuvirtide, T-20 | Roche |
| | | | Pharmaceuticals |
| | | | & Trimeris |

Entry Inhibitors

| | | | |
|---|---|---|---|
| 2007 | Selzentry | Maraviroc | Pfizer |

Integrase Inhibitors

| | | | |
|---|---|---|---|
| 2007 | Isentress | Raltegravir | Merck |
| 2013 | Tivicay | Dolutegravir | ViiV Healthcare |
| 2018 | | Bictegravir | Gilead Sciences |
| — | — | Cabotegravir | ViiV Healthcare |

Capsid Inhibitors

| | | |
|---|---|---|
| — | GS-6207 | Gilead Sciences |

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

The present invention may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350, and SPI-452. Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott park, Illinois, as Norvir. Ritonavir is an HIV protease inhibitor indicated with other antiretroviral agents for the treatment of HIV infection. Ritonavir also inhibits P450 mediated drug metabolism as well as the P-gycoprotein (Pgp) cell transport system, thereby resulting in increased concentrations of active compound within the organism.

GS-9350 is a compound being developed by Gilead Sciences of Foster City Calif. as a pharmacological enhancer.
SPI-452 is a compound being developed by Sequoia Pharmaceuticals of Gaithersburg, Md., as a pharmacological enhancer.

In one embodiment of the present invention, a compound of Formula (I) is used in combination with ritonavir. In one embodiment, the combination is an oral fixed dose combination. In another embodiment, the compound of Formula (I) is formulated as a long acting parenteral injection and ritonavir is formulated as an oral composition. In one embodiment, a kit containing the compound of Formula (I) is formulated as a long acting parenteral injection and ritonavir formulated as an oral composition. In another embodiment, the compound of Formula (I) is formulated as a long acting parenteral injection and ritonavir is formulated as an injectable composition. In one embodiment, a kit containing the compound of Formula (I) is formulated as a long acting parenteral injection and ritonavir formulated as an injectable composition.

In another embodiment of the present invention, a compound of Formula (I) is used in combination with GS-9350. In one embodiment, the combination is an oral fixed dose combination. In another embodiment, the compound of Formula (I) is formulated as a long acting parenteral injection and GS-9350 is formulated as an oral composition. In one embodiment, there is provided a kit containing the compound of Formula (I) is formulated as a long acting parenteral injection and GS-9350 formulated as an oral composition. In another embodiment, the compound of Formula (I) is formulated as a long acting parenteral injection and GS-9350 is formulated as an injectable composition. In one embodiment, is a kit containing the compound of Formula (I) is formulated as a long acting parenteral injection and GS-9350 formulated as an injectable composition.

In one embodiment of the present invention, a compound of Formula (I) is used in combination with SPI-452. In one embodiment, the combination is an oral fixed dose combination. In another embodiment, the compound of Formula (I) is formulated as a long acting parenteral injection and SPI-452 is formulated as an oral composition. In one embodiment, there is provided a kit containing the compound of Formula (I) formulated as a long acting parenteral injection and SPI-452 formulated as an oral composition. In another embodiment, the compound of Formula (I) is formulated as a long acting parenteral injection and SPI-452 is formulated as an injectable composition. In one embodiment, there is provided a kit containing the compound of Formula (I) formulated as a long acting parenteral injection and SPI-452 formulated as an injectable composition.

In one embodiment of the present invention, a compound of Formula (I) is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I).

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), wherein said virus is an HIV virus. In some embodiments, the HIV virus is the HIV-1 virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I) further comprising administration of a therapeutically effective amount of one or more agents active against an HIV virus.

In another embodiment of the invention, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CAPSID inhibitors, CXCR4 inhibitors; and CCR5 inhibitors.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I).

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), wherein said virus is an HIV virus. In some embodiments, the HIV virus is the HIV-1 virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against an HIV virus.

In another embodiment of the invention, there is provided a method for preventing a viral infection in a mammal mediated at least in part by a virus in the retrovirus family of viruses which method comprises administering to a mammal, that has been diagnosed with said viral infection or is at risk of developing said viral infection, a compound of Formula (I), further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CAPSID inhibitors, CXCR4 inhibitors; and CCR5 inhibitors.

In further embodiments, the compound of the present invention of Formula (I) or a pharmaceutically acceptable salt thereof, is selected from the group of compounds set forth in Table 1 above.

The compounds of Table 1 were synthesized according to the Synthetic Methods, General Schemes, and the Examples described below.

In another embodiment, there is provided a pharmaceutical composition comprising a pharmaceutically acceptable diluent and a therapeutically effective amount of a compound of Formula (I) or a pharmaceutically acceptable salt thereof.

In certain embodiments, the compound(s) of the present invention, or a pharmaceutically acceptable salt thereof, is chosen from the compounds set forth in Table 1.

The compounds of Formula (I) of the invention may exist in both unsolvated and solvated forms. The term ‘solvate’ comprises the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Compounds of Formula (I) containing one or more asymmetric carbon atoms can exist as two or more stereoisomers. Where a compound of Formula (I) contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC), Supercritical fluid chromatography (SFC).

Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC or SFC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds of Formula (I) can generally be prepared by conventional techniques known to those skilled in the art or by processes analogous to those described herein using an appropriate isotopically-labelled reagents in place of the non-labelled reagent previously employed.

The compounds of the present invention may be administered as prodrugs. Thus, certain derivatives of compounds of Formula (I), which may have little or no pharmacological activity themselves can, when administered into or onto the body, be converted into compounds of Formula (I) as ‘prodrugs’. One example of a compound that such prodrugs may encompass is 4′-ethylnyl-2-fluoro-2′-dooxyadenosine (EFdA) disclosed e.g., in U.S. Pat. No. 7,339,053. The compounds of the present invention may be administered as prodrugs. In one embodiment, the compounds of the invention are prodrugs of 4′-ethynyl-2-fluoro-2′-deoxyadenosine (EFdA) disclosed e.g., in U.S. Pat. No. 7,339,053, which is a nucleoside reverse transcriptase inhibitor of the formula:

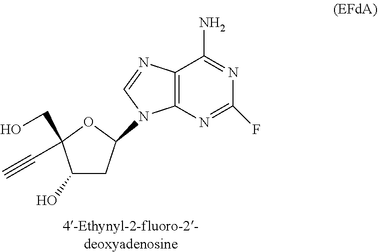 [SMILES: C#C[C@]1(CO)O[C@@H](n2cnc3c(N)nc(F)nc32)C[C@@H]1O]

The prodrugs are useful in that they are believed to be capable of modulating physicochemical properties, facilitating multiple dosing paradigms and improving pharmacokinetic and/or pharmacodynamic profiles of the active parent (EfdA). For example, the prodrugs may facilitate long-acting parenteral dosing modalities, and/or improvements in antiviral persistence profiles as compared to EFdA.

Administration of the chemical entities and combinations of entities described herein can be via any of the accepted modes of administration for agents that serve similar utilities including, but not limited to, orally, sublingually, subcutaneously, intravenously, intranasally, topically, transdermally, intraperitoneally, intramuscularly, intrapulmonarilly, vaginally, rectally, or intraocularly. In some embodiments, oral or parenteral administration is used. Examples of dosing include, without limitation, once every seven days for oral, once every eight weeks for intramuscular, or once every six months for subcutaneous.

Pharmaceutical compositions or formulations include solid, semi-solid, liquid and aerosol dosage forms, such as, e.g., tablets, capsules, powders, liquids, suspensions, suppositories, aerosols or the like. The chemical entities can also be administered in sustained or controlled release dosage forms, including depot injections, osmotic pumps, pills, transdermal (including electrotransport) patches, and the like, for prolonged and/or timed, pulsed administration at a predetermined rate. In certain embodiments, the compositions are provided in unit dosage forms suitable for single administration of a precise dose.

The chemical entities described herein can be administered either alone or more typically in combination with a conventional pharmaceutical carrier, excipient or the like (e.g., mannitol, lactose, starch, magnesium stearate, sodium saccharine, talcum, cellulose, sodium crosscarmellose, glucose, gelatin, sucrose, magnesium carbonate, and the like). If desired, the pharmaceutical composition can also contain minor amounts of nontoxic auxiliary substances such as wetting agents, emulsifying agents, solubilizing agents, pH buffering agents and the like (e.g., sodium acetate, sodium citrate, cyclodextrine derivatives, sorbitan monolaurate, triethanolamine acetate, triethanolamine oleate, and the like). Generally, depending on the intended mode of administration, the pharmaceutical composition will contain about 0.005% to 95%; in certain embodiments, about 0.5% to 50% by weight of a chemical entity. Actual methods of preparing such dosage forms are known, or will be apparent, to those skilled in this art; for example, see Remington's Pharmaceutical Sciences, Mack Publishing Company, Easton, Pa.

In certain embodiments, the compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils or triglycerides) is encapsulated in a gelatin capsule.

Liquid pharmaceutically administrable compositions can, for example, be prepared by dissolving, dispersing, etc. at least one chemical entity and optional pharmaceutical adjuvants in a carrier (e.g., water, saline, aqueous dextrose, glycerol, glycols, ethanol or the like) to form a solution or suspension. Injectables can be prepared in conventional forms, either as liquid solutions or suspensions, as emulsions, or in solid forms suitable for dissolution or suspension in liquid prior to injection. The percentage of chemical entities contained in such parenteral compositions is highly dependent on the specific nature thereof, as well as the activity of the chemical entities and the needs of the subject. However, percentages of active ingredient of 0.01% to 10% in solution are employable, and will be higher if the composition is a solid which will be subsequently diluted to the above percentages. In certain embodiments, the composition may comprise from about 0.2 to 2% of the active agent in solution.

Pharmaceutical compositions of the chemical entities described herein may also be administered to the respiratory tract as an aerosol or solution for a nebulizer, or as a microfine powder for insufflation, alone or in combination with an inert carrier such as lactose. In such a case, the particles of the pharmaceutical composition have diameters of less than 50 microns, in certain embodiments, less than 10 microns.

In general, the chemical entities provided will be administered in a therapeutically effective amount by any of the accepted modes of administration for agents that serve similar utilities. The actual amount of the chemical entity, i.e., the active ingredient, will depend upon numerous factors such as the severity of the disease to be treated, the age and relative health of the subject, the potency of the chemical entity used the route and form of administration, and other factors. The drug can be administered more than once a day, such as once or twice a day.

In general, the chemical entities will be administered as pharmaceutical compositions by any one of the following routes: oral, systemic (e.g., transdermal, intranasal or by suppository), or parenteral (e.g., intramuscular, intravenous or subcutaneous) administration. In certain embodiments, oral administration with a convenient daily dosage regimen that can be adjusted according to the degree of affliction may be used. Compositions can take the form of tablets, pills, capsules, semisolids, powders, sustained release formulations, solutions, suspensions, elixirs, aerosols, or any other appropriate compositions. Another manner for administering the provided chemical entities is inhalation.

The choice of formulation depends on various factors such as the mode of drug administration and bioavailability of the drug substance. For delivery via inhalation the chemical entity can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDIs typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical compositions have been developed for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, at least one chemical entity described herein in combination with at least one pharmaceutically acceptable excipient. Acceptable excipients are non-toxic, aid administration, and do not adversely affect the therapeutic benefit of the at least one chemical entity described herein. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, for injectable solutions, include water, saline, aqueous dextrose, and glycols.

Compressed gases may be used to disperse a chemical entity described herein in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the chemical entity in a composition can vary within the full range employed by those skilled in the art. Typically, the composition will contain, on a weight percent (wt %) basis, from about 0.01-99.99 wt % of at least one chemical entity described herein based on the total composition, with the balance being one or more suitable pharmaceutical excipients. In certain embodiments, the at least one chemical entity described herein is present at a level of about 1-80 wt %.

In various embodiments, pharmaceutical compositions of the present invention encompass compounds of Formula (I), salts thereof, and combinations of the above.

### Synthetic Methods

The methods of synthesis may employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The compounds of Formula (I) herein including those in Examples 1-72 contain a phosphorus chiral center. The isomer mixture were separated, providing an Isomer #A e.g. Isomer 1A (faster eluting isomer) and an Isomer #B, e g. Isomer 1B (slower eluting isomer), based on their observed elution order resulting from the separation as performed in the Example. Where retention times are shown, they are provided only to show, the relative order of elution of each isomer in an Example. Elution order of separated isomers may differ if performed under conditions different than those employed herein. Absolute stereochemistry (R or S) of the phosphorus chiral center in each of the “A” and “B” stereoisomers in Examples 1 to 72 was not determined. An asterisk (*) may be used in the associated chemical structure drawings of the Example compounds to indicate the phosphorus chiral center.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Ernka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein may take place at atmospheric pressure, generally within a temperature range from −78° C. to 200° C. Further, except as employed in the Example or as otherwise specified, reaction times and conditions are intended to be approximate, e.g., taking place at about atmospheric pressure within a temperature range of about −78° C. to about 110° C. over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein can be affected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### EXAMPLE AND GENERAL SYNTHESIS

The following example and prophetic synthesis method serve to more fully describe the manner of making and using the above-described invention. It is understood that this in no way serve to limit the true scope of the invention, but rather is presented for illustrative purposes. Unless otherwise specified, the following abbreviations have the following meanings. If an abbreviation is not defined, it has its generally accepted meaning.
aq.=aqueous
μL=microliters
μM=micromolar
NMR=nuclear magnetic resonance
Boc=tert-butoxycarbonyl
br=broad
BQL=below quantification limit
Cbz=benzyloxycarbonyl
d=doublet
° C.=degrees Celsius
DCM=dichloromethane
DIPEA=N,N-diisopropylethylamine
dd=doublet of doublets
DMAP=N,N-dimethylaminopyridine
DMEM=Dulbeco's Modified Eagle's Medium
DMF=N,N-dimethylformamide
DMSO=dimethylsufoxide
EDC=N-(3-Dimethylaminopropyl)-N′-ethylcarbodiimide hydrochloride
EtOAc=ethyl acetate
g=gram
h or hr=hour(s)
HPLC=high performance liquid chromatography
Hz=Hertz
IU=International Units
IC₅₀=50% inhibitory concentration
J=coupling constant in Hz
LCMS=liquid chromatography mass spectrometry
m=multiplet
M=molar concentration
M+H⁺=parent mass spectrum peak plus H⁺
mg=milligram
min=minute(s)
mL=milliliter
mM=millimolar
mmol=millimole
MMTr=monomethoxytrityl
MS=mass spectrum
MTBE=methyl tert-butyl ether
nM=nanomolar
PE=petroleum ether
ppm=parts per million
q.s.=sufficient amount
s=singlet
RT=retention time
sat.=saturated
t=triplet
TBDMS=tert-butyldimethylsilyl
TBDPS=tert-butyldiphenylsilyl
TEA=triethylamine
THF=tetrahydrofuran
TMS=trimethylsilyl

Additionally, various compounds of the invention may be made, in one embodiment, by way of the general synthesis routes set forth in Schemes 1-2 below:

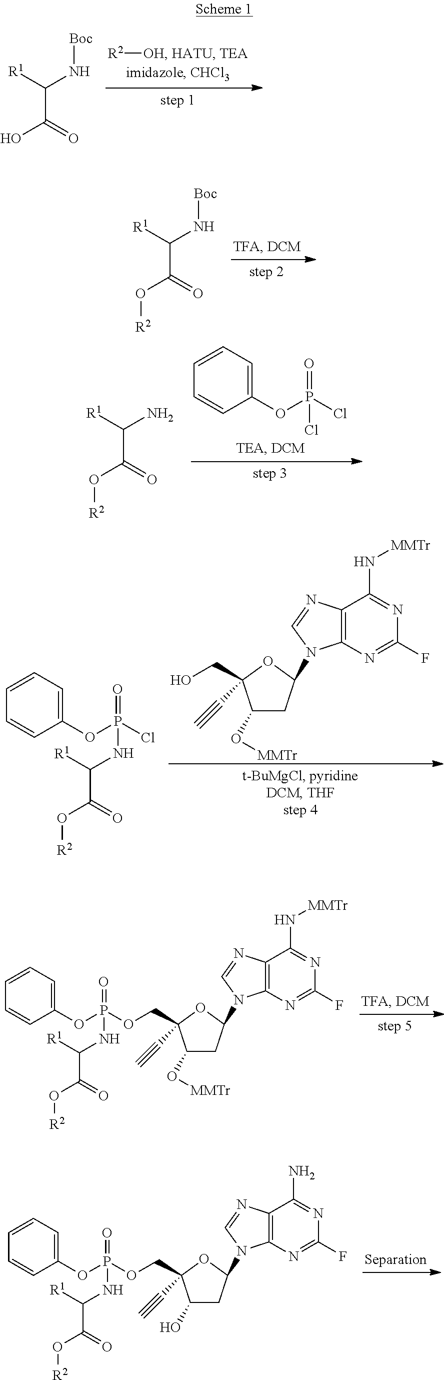

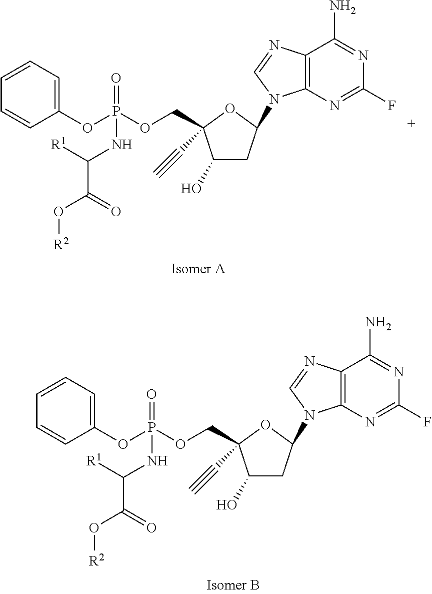

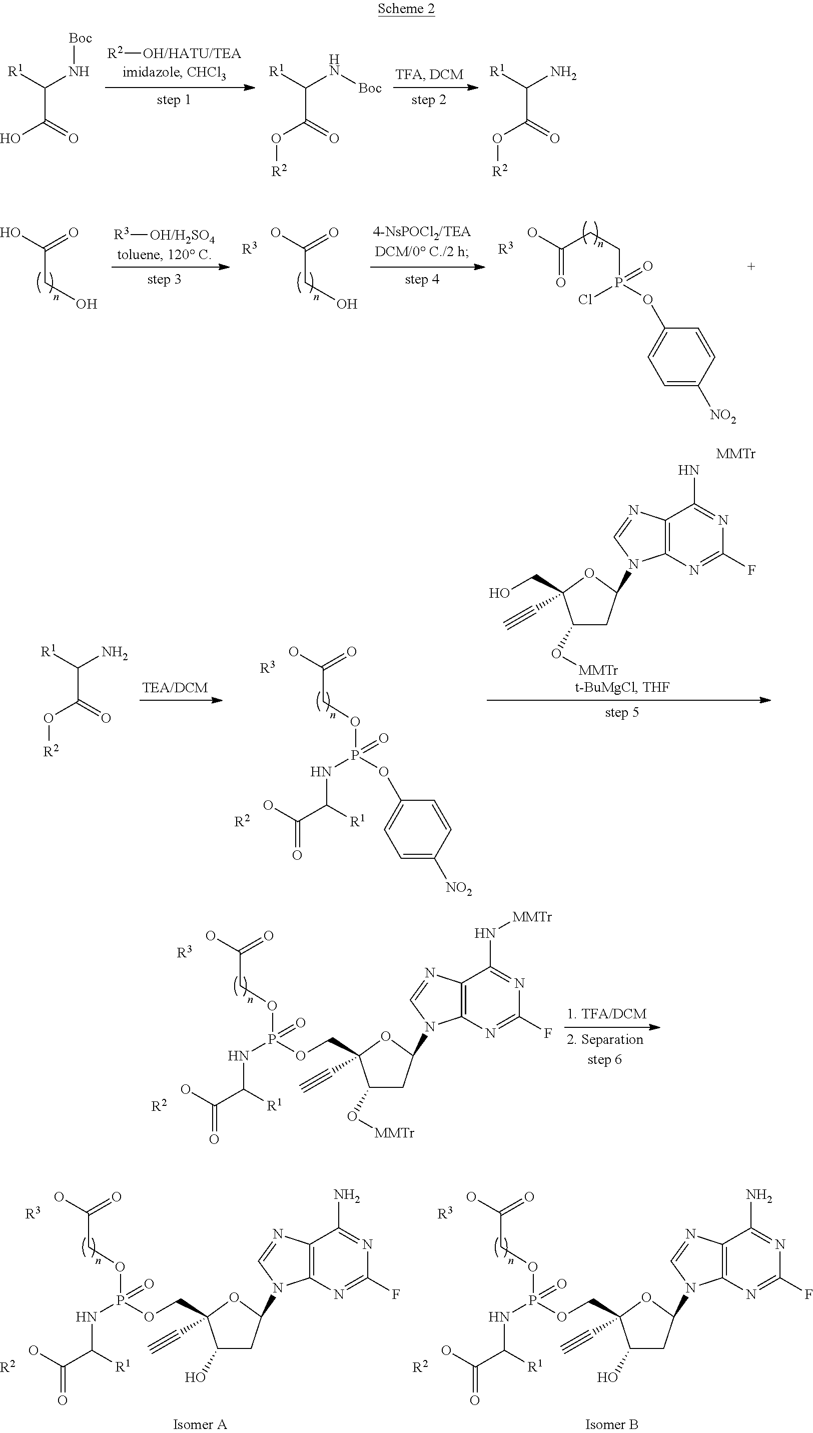

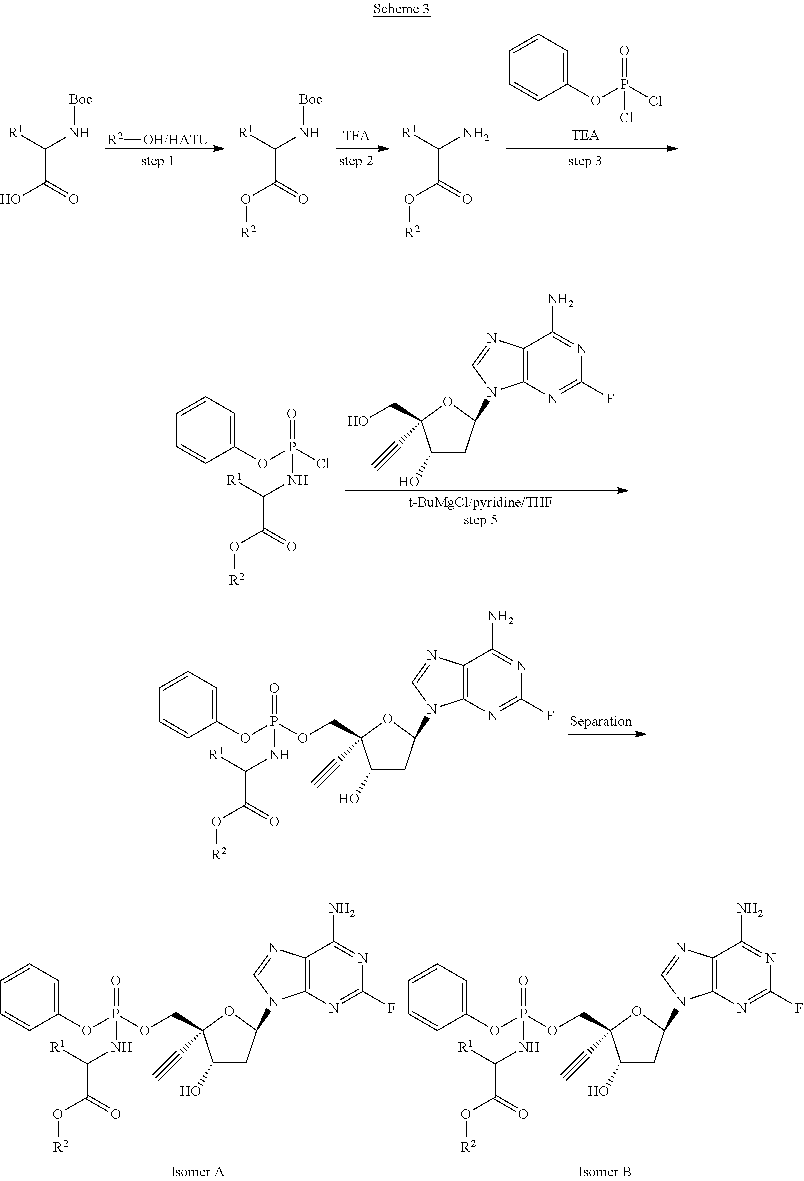

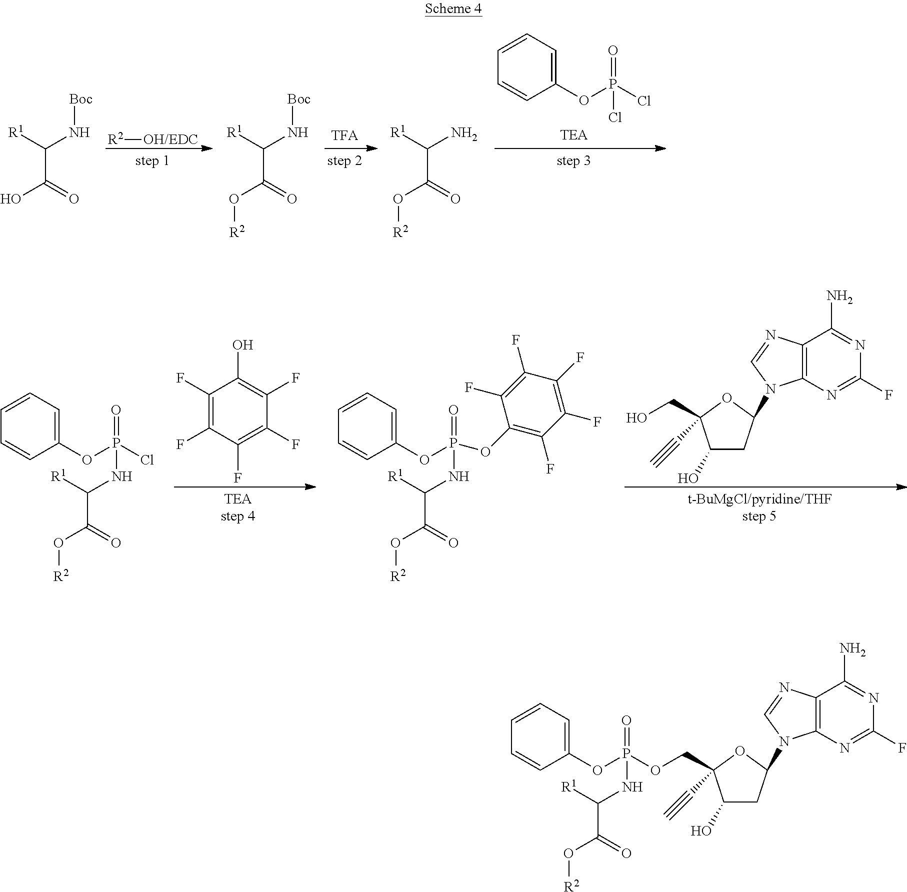

Absolute stereochemistry (R or S) of the phosphorus chiral center in each of the “A and “B” stereoisomers in Schemes 1 and 2 was not determined.

In Scheme 2, n is from 1 to 10, and each of, R¹, R² and R³ are alkyl, alkylaryl, or aryl.

### Example 1

### Docosyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

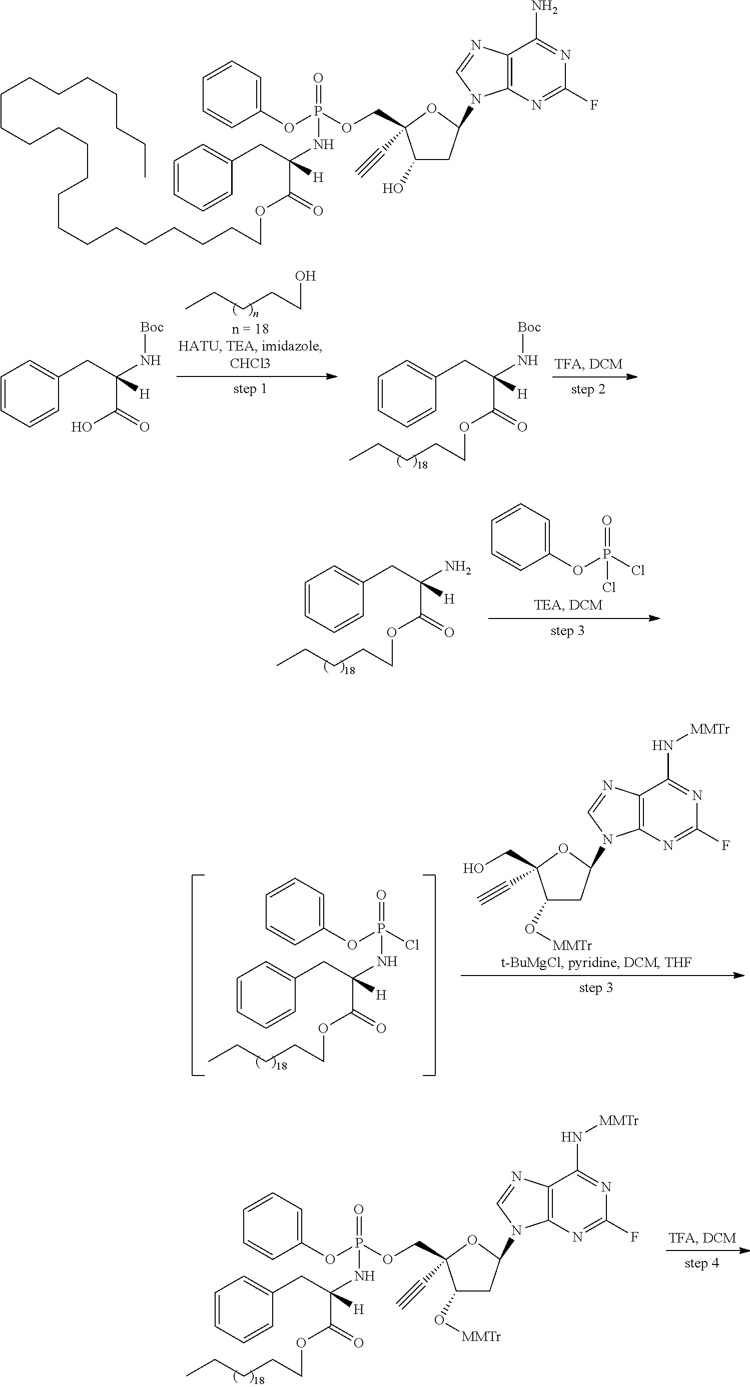

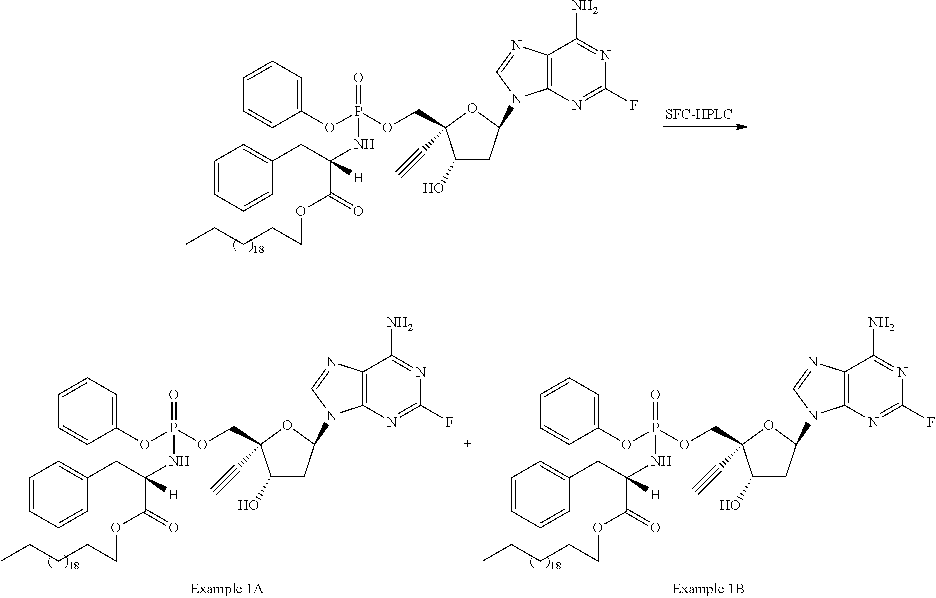

### Step 1: Docosyl (tert-butoxycarbonyl)-L-phenylalaninate

To a solution of (tert-butoxycarbonyl)-L-phenylalanine (10 g, 37.7 mmol), HATU (28.7 g, 75 mmol) and icosan-1-ol (16.88 g, 56.5 mmol) in chloroform (100 mL) stirred under nitrogen at 25° C. was added 1H-imidazole 7.7 g (113 mmol) and DIEA (19.75 mL, 113 mmol) in chloroform (100 mL). The reaction mixture was stirred at 25° C. for 24 hours. LCMS indicated completion of reaction. The reaction mixture was diluted with DCM (100 mL), washed with water (100 mL), brine (100 mL), dried over anhydrous sodium sulfate and concentrated to dryness in vaccum. The crude product was purified by flash column chromatography (silica gel, 500 g, pet. ether:EtOAc=20:1) to give the title compound (14 g, 97%, yield: 62.8%) as a white solid. LCMS (ESI) m/z calcd for C₃₆H₆₃NO₄: 573; found: 574 (M+1). ¹H NMR (400 MHz, Chloroform-d) δ 7.28 (dd, J=8.0, 6.0 Hz, 2H), 7.23 (dd, J=8.4, 6.0 Hz, 1H), 7.16-7.10 (m, 2H), 4.97 (d, J=8.4 Hz, 1H), 4.74-4.33 (m, 1H), 4.08 (td, J=6.8, 1.6 Hz, 2H), 3.09 (q, J=7.6 Hz, 2H), 1.57 (d, J=5.2 Hz, 2H), 1.42 (s, 9H) 1.26 (s, 38H), 0.88 (t, J=6.8 Hz, 3H).

### Step 2: Docosyl L-phenylalaninate

To a solution of docosyl (tert-butoxycarbonyl)-L-phenylalaninate (14 g, 24.39 mmol) in DCM (140 mL) stirred at 25° C. was added TFA (46 ml, 24.39 mmol) dropwise. The reaction mixture was stirred at 25° C. for 1 hour. LCMS indicated completion of reaction. The reaction was quenched with aqueous NaHCO₃ and pH was adjusted to −8 and the organic layer was separated. The aqueous phase was extracted with DCM (300 mL*3). The combined organic layers were washed with brine, dried over anhydrous sodium sulfate and concentrated to dryness in vaccum to give title compound (11 g, 90%, yield: 86%) as a white solid. LCMS (ESI) m/z calcd for C₃₁H₅₅NO₂: 473; found: 474 (M+1). ¹H NMR (400 MHz, Chloroform-d) δ 7.33-7.17 (m, 5H), 4.09 (t, J=6.8 Hz, 2H), 3.72 (dd, J=7.6, 5.2 Hz, 1H), 3.13-2.82 (m, 2H), 1.65-1.49 (m, 4H), 1.25 (s, 38H), 0.88 (t, J=6.8 Hz, 3H).

### Step 3: Docosyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methoxy)(phenoxy)phosphoryl)-L-phenylalaninate

To a solution of docosyl L-phenylalaninate (1.5 g, 3.17 mmol) and triethylamine (1.282 g, 12.66 mmol) in DCM (15 mL) stirred under nitrogen at 0° C. was added phenyl phosphorodichloridate (0.668 g, 3.17 mmol) portionwise. The reaction mixture was stirred at 25° C. for 1 hour. To a solution of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (2.65 g, 3.17 mmol) in pyridine (15.00 mL)/THF (15.000 mL) stirred under nitrogen at 25° C. was added a solution of tert-butylmagnesium chloride (4.43 mL, 4.43 mmol, 1 N) in THF dropwise. The reaction mixture was stirred at 25° C. for 0.5 hour. To this reaction mixture was added above reaction mixture at 25° C. and the resulting mixture was stirred at 25° C. for 1 hour. LCMS indicated completion of reaction. The reaction was quenched with water (50 mL) and the organic layer was separated. The aqueous phase was extracted with DCM (20 ml*3). The combined organic layers were washed with brine (20 mL), dried over anhydrous sodium sulfate and concentrated to dryness in vaccum. The crude product was purified by flash column chromatography (silica gel, 330 g, pet. ether:EtOAc=5:3) to give the title compound (1.2 g, 80%, yield: 20.9%) as white solid. LCMS (ESI) m/z calcd for C₈₉H₁₀₂FN₆O₉P: 1449; found: 1450 (M+1). ¹H NMR (400 MHz, Chloroform-d) δ 8.61-8.54 (m, 1H), 7.94 (d, J=9.6 Hz, 1H), 7.52-7.36 (m, 5H), 7.35-7.24 (m, 12H), 7.24-6.98 (m, 15H), 6.92-6.76 (m, 6H), 6.17-5.90 (m, 2H), 4.41 (q, J=6.0 Hz, 1H), 4.00-3.72 (m, 5H), 3.69 (d, J=1.6 Hz, 3H), 3.65 (d, J=1.6 Hz, 3H), 3.52 (dd, J=10.8, 4.2 Hz, 1H), 2.91-2.67 (m, 2H), 1.68 (d, J=7.6 Hz, 2H), 1.32-1.24 (m, 2H), 1.19 (d, J=6.0 Hz, 38H), 0.88-0.74 (m, 3H).

### Step 4: Docosyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

To a solution of docosyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)meth-oxy)(phenoxy)phosphoryl)-L-phenylalaninate (1.1 g, 0.759 mmol) in DCM (11 mL) stirred at room 25° C. was added trifluoroacetic acid (TFA) (1.1 mL). After 2 hours at 25° C., LCMS indicated completion of. reaction. The reaction was quenched with saturated aqueous NaHCO₃ and pH was adjusted to 8. The organic layer was separated, dried over anhydrous sodium sulfate and concentrated to dryness in vacuum. The crude product was purified by flash column chromatography (silica gel, 40 g, pet. ether:EtOAc=5:2) to give the product (640 mg) as a white solid. The diastereomers were separated with Prep-SFC (Column: CHIRALPAK IH, 2*25 cm, 5 μm; Mobile Phase A:CO₂, Mobile Phase B:MeOH-Preparative; Flow rate: 40 mL/min; Gradient: 40% B; 254 nm; RT₁: 3.74; RT₂: 6.7; Injection Volumn: 4.5 ml; Number of Runs: 9 to give two isomers. First eluting isomer (Example 1A, RT1: 3.74 min, 133.9 mg, 99.19%, yield: 19.34%) obtained as an off-white solid; LCMS (ESI) m/z calcd for C₄₉H₇₀FN₆O₇P: 905; found: 906 (M+1); ¹H NMR (400 MHz, Chloroform-d) 67.97 (s, 1H), 7.28-7.26 (m, 1H), 7.24-7.09 (m, 9H), 6.33-6.05 (m, 3H), 4.72-4.70 (m, 1H), 4.27-4.21 (m, 3H), 4.05-3.96 (m, 2H), 3.70 (br, 1H), 3.00-2.99 (d, J=6 Hz, 2H), 2.73-2.70 (m, 3H), 1.54-1.50 (m, 2H), 1.30-1.23 (m, 38H), 0.88 (t, J=6.8 Hz, 3H); and second eluting isomer (Example 1B, RT2: 6.7 min, 247.2 mg, 99.33%, yield: 35.8%) obtained as an off-white solid; LCMS (ESI) m/z calcd for C₄₉H₇₀FN₆O₇P: 905; found: 906 (M+1); ¹H NMR (400 MHz, Chloroform-d) δ7.82 (s, 1H), 7.32-7.26 (m, 3H), 7.24-7.22 (m, 2H), 7.17-7.15 (m, 3H), 7.08-7.07 (m, 2H), 6.32-6.31 (m, 1H), 6.1 (br s, 1H), 4.65-4.60 (m, 1H), 4.20-4.17 (m, 2H), 4.04 (t, J=6 Hz, 2H), 3.99-3.90 (m, 1H), 3.68-3.55 (m, 2H), 3.00-2.96 (m, 2H), 2.71 (s, 1H), 2.70-2.50 (m, 2H), 1.60-1.50 (m, 2H), 1.30-1.20 (m, 38H), 0.88 (t, J=6.8 Hz, 3H).

### Example 2

### Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

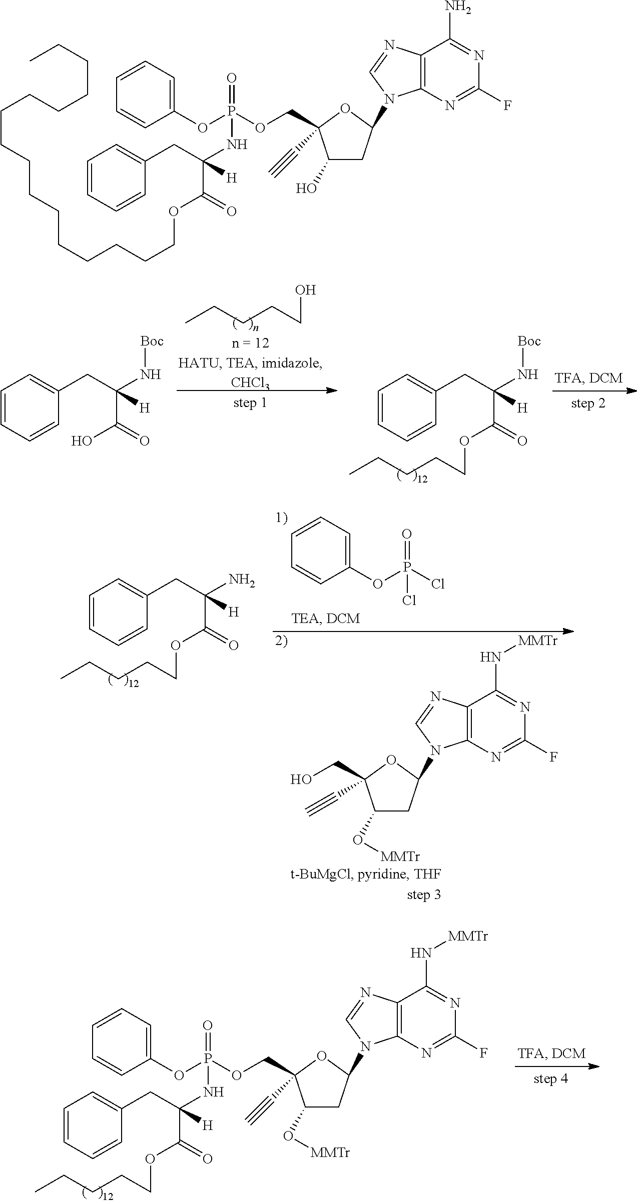

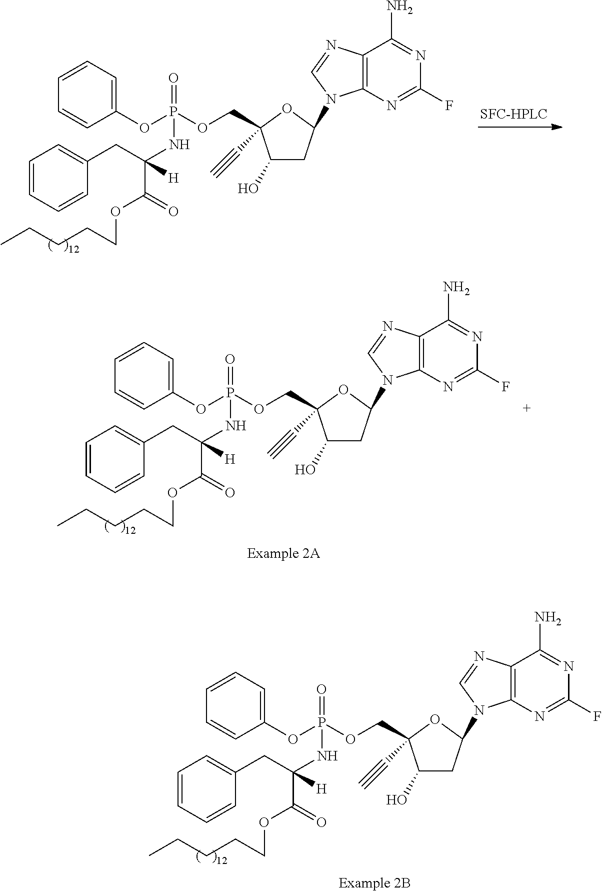

### Step 1: Hexadecyl (tert-butoxycarbonyl)-L-phenylalaninate

To a stirred mixture of (tert-butoxycarbonyl)-L-phenylalanine (10 g, 37.7 mmol) in chloroform (70 mL) was added HATU (21.50 g, 56.5 mmol), hexadecan-1-ol (9.14 g, 37.7 mmol), TEA (15.76 mL, 113 mmol) and imidazole (7.70 g, 113 mmol) at 0° C. The reaction mixture was stirred for 16 h at room temperature. LCMS indicated completion of. reaction. The reaction mixture was quenched with water (100 mL) and extracted with DCM (100 mL*2). The combined organic phases were washed with brine (30 mL), dried over Na2SO4 and concentrated under vacuum. The residue was purified by flash column chromatography (silica gel, 330 g, pet. ether:EA=10:1) to give hexadecyl (tert-butoxycarbonyl)-L-phenylalaninate (8.5 g, 100% purity, 46.0% yield) as a yellow oil. LCMS (ESI) m/z calcd for C₃₀H₅₁NO₄:489; found: 490 (M+H). ¹H NMR (400 MHz, CDCl₃) δ 7.38-7.12 (m, 5H), 5.00 (d, J=11.2 Hz, 1H), 4.65-4.52 (m, 1H), J=8.8 Hz, 2H), 3.18-2.98 (m, 2H), 1.71-1.52 (m, 4H), 1.50-1.19 (m, 33H), 0.97-0.83 (m, 3H).

### Step 2: Hexadecyl L-phenylalaninate

To a stirred mixture of hexadecyl (tert-butoxycarbonyl)-L-phenylalaninate (8 g, 16.34 mmol) in DCM (50 mL) was added TFA (15.0 mL, 195 mmol) at 0° C. The reaction mixture was stirred for 1 h at room temperature. LCMS indicated completion of. reaction. The reaction mixture was quenched with NaHCO₃ (100 mL). and extracted with DCM (80 mL×2). The combined organic phases were washed with brine (15 mL), dried over Na2SO4 and concentrated under vacuum to give hexadecyl L-phenylalaninate (6 g, 100% purity, 94 yield) as a white solid. LCMS (ESI) m/z calcd for C₂₅H₄₃NO₂: 389; found: 390 (M+H). ¹H NMR (400 MHz, CDCl₃) δ 7.32-7.15 (m, 5H), 4.08 (t, J=6.4 Hz, 2H), 3.71 (dd, J=5.6, 8.0 Hz, 1H), 3.07 (dd, J=5.6, 13.6 Hz, 1H), 2.86 (dd, J=7.6, 13.6 Hz, 1H), 1.63-1.48 (m, 4H), 1.34-1.17 (m, 24H), 0.88 (t, J=6.8 Hz, 3H).

### Step 3: Hexadecyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(phenoxy)phosphoryl)-L-phenylalaninate

To a solution of hexadecyl L-phenylalaninate (2 g, 5.13 mmol) and triethylamine (2.078 g, 20.53 mmol) in DCM (20 mL) stirred under nitrogen at 0° C. was added phenyl phosphorodichloridate (1.083 g, 5.13 mmol) portionwise. The reaction mixture was stirred at room temperature for 1 hour.

To a solution of. ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (4.30 g, 5.13 mmol) in pyridine (20.0 mL)/THF (20.000 mL) stirred under nitrogen at room temperature was added a solution of tert-butylmagnesium chloride (7.19 mL, 7.19 mmol) in THF dropwise. The reaction mixture was stirred at 25° C. for 0.5 hour. To this reaction mixture was added above reaction mixture at room temperature and stirred for 16 hours. LCMS indicated completion of reaction. The reaction mixture was evaporated under vacuum to give the crude product as a red oil. The oil was purified by silica column (120 g) using a 0%-60% EtOAc/pet. ether solvent gradient to give hexadecyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(phenoxy) phosphoryl)-L-phenylalaninate (2 g, 90% purity, 28.5% yield) as pale yellow oil. LCMS (ESI) m/z calcd for C₈₃H₉₀FN₆O₉P: 1365; found: 1366 (M+1).

### Step 4: Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of hexadecyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(phenoxy)phosphoryl)-L-phenylalaninate (2 g, 1.465 mmol) in DCM (8 mL) stirred at room temperature was added TFA (1 mL) dropwise. The reaction mixture was stirred at room temperature for 30 min. LCMS indicated completion of. reaction. The reaction mixture was quenched with NaHCO₃ (60 mL) and extracted with DCM (60 mL×2). The combined organic phases were washed with brine (15 mL), dried over Na₂SO₄ and concentrated under vacuum. The residue was purified by gel silica column (80 g, pet. ether:EA=1:2) to give the title compound (1 g) as colorless semisolid. The diastereomers were separated by Prep-SFC-HPLC with the following conditions: Column: CHIRALPAK IH, 50*250 mm; Mobile Phase A: CO2, Mobile Phase B: MEOH (2 mM NH3-MEOH); Flow rate: 180 mL/min; Gradient: 50% B; 220 nm; RT1: 3.91; RT2: 6.75; Injection Volumn: 6 ml; number of runs: 3; to give first eluting isomer (Example 1A, RT1: 3.91 min, 415.9 mg, 98.0% purity, 33.9% yield) obtained as white solid; LCMS (ESI) m/z calcd for C₄₃H₅₈FN₆O₇P: 820; found: 821 (M+1); ¹H NMR (400 MHz, Chloroform-d) δ 8.02 (s, 1H), 7.34-7.08 (m, 10H), 6.32 (dd, J=3.6, 7.2 Hz, 1H), 4.56 (t, J=7.6 Hz, 1H), 4.26-4.11 (m, 2H), 4.05 (t, J=6.8 Hz, 2H), 3.92 (dd, J=9.2, 12.0 Hz, 1H), 3.60 (t, J=10.8 Hz, 1H), 3.46 (br, s, 1H), 3.07-2.90 (m, 2H), 2.75-2.64 (m, 2H), 2.61-2.51 (m, 1H), 1.60-1.51 (m, 2H), 1.33-1.18 (m, 26H), 0.87 (t, J=6.4 Hz, 3H), and the second eluting isomer (Example 1B RT2: 6.75 min, 252.6 mg, 98.05% purity, 20.60% yield) obtained as white solid; LCMS (ESI) m/z calcd for C₄₃H₅₈FN₆O₇P: 820; found: 821 (M+1); ¹H NMR (400 MHz, Chloroform-d) δ 8.17 (s, 1H), 7.29-7.08 (m, 10H), 6.32 (t, J=5.6 Hz, 1H), 4.68 (t, J=7.6 Hz, 1H), 4.30-4.17 (m, 3H), 4.10-4.02 (m, 2H), 3.82 (t, J=10.8 Hz, 1H), 3.60 (t, J=10.8 Hz, 1H), 3.01 (d, J=6.4 Hz, 2H), 2.75-2.66 (m, 3H), 1.59-1.48 (m, 2H), 1.34-1.18 (m, 26H), 0.87 (t, J=6.8 Hz, 3H).

### Example 3

### Dodecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

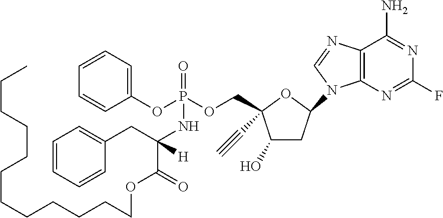 [SMILES: C#C[C@]1(COP(=O)(N[C@@H](Cc2ccccc2)C(=O)OCCCCCCCCCCCC)Oc2ccccc2)O[C@@H](n2cnc3c(N)nc(F)nc32)C[C@@H]1O]

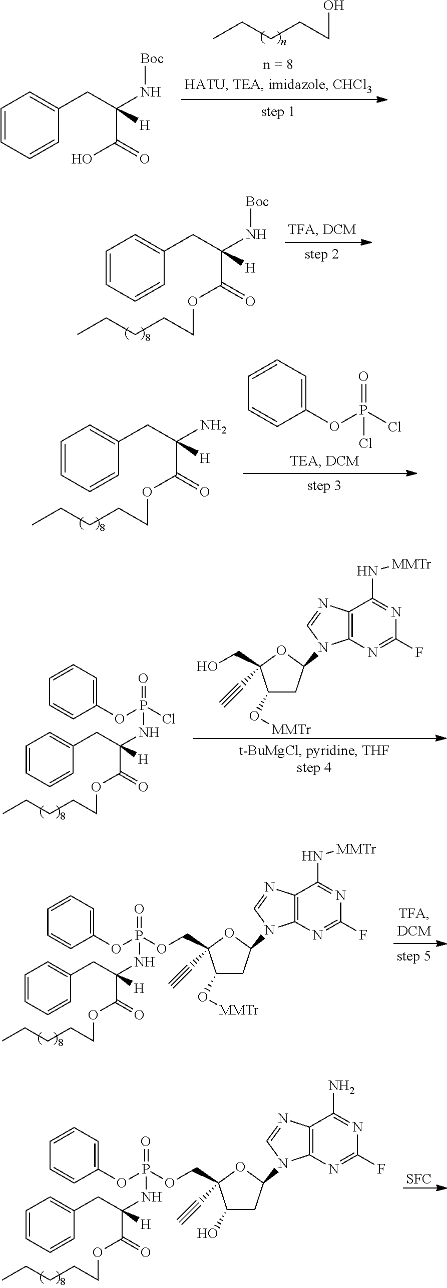

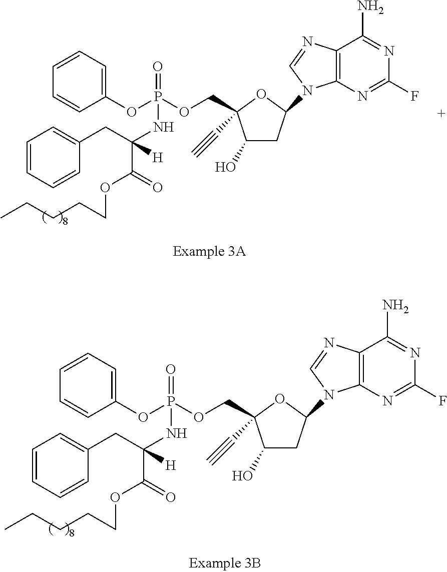

### Step 1: Dodecyl (tert-butoxycarbonyl)-L-phenylalaninate

To a solution of (tert-butoxycarbonyl)-L-phenylalanine (5 g, 18.85 mmol) and HATU (14.33 g, 37.7 mmol) in chloroform (50 mL) stirred at 25° C. was added dodecan-1-ol (5.27 g, 28.3 mmol), 1H-imidazole (3.85 g, 56.5 mmol), N-ethyl-N-isopropylpropan-2-amine (7.31 g, 56.5 mmol). The reaction mixture was stirred at 25° C. for 18 hours. LCMS indicated completion of. reaction. The reaction was washed with water (30 mL*2), brine (30 mL), dried over anhydrous sodium sulfate and concentrated to dryness under vaccum. The crude product was purified by flash column chromatography (silica gel, 330 g, pet. ether:EA=7:3) to give the title compound (6.8 g, 100%, yield: 83%) as colorless oil. LCMS (ESI) m/z calcd for C₂₆H₄₃NO₄: 433; found: 434 (M+1). ¹H NMR (300 MHz, Chloroform-d) δ 7.36-7.22 (m, 3H), 7.22-7.12 (m, 2H), 5.00 (d, J=8.4 Hz, 1H), 4.59 (d, J=7.5 Hz, 1H), 4.16-4.05 (m, 2H), 3.10 (t, J=5.4 Hz, 2H), 1.61 (d, J=6.0 Hz, 2H), 1.44 (s, 9H), 1.33-1.27 (m, 18H), 0.96-0.85 (m, 3H).

### Step 2: Docosyl L-phenylalaninate

To a solution of dodecyl (tert-butoxycarbonyl)-L-phenylalaninate (6.3 g, 14.53 mmol) in DCM (60 mL) stirred in air at 25° C. was added TFA (20 mL, 260 mmol). The reaction mixture was stirred at 25° C. for 30 minutes. LCMS indicated completion of. reaction. The reaction was quenched with saturated aqueous NaHCO₃ and pH was adjusted to 8 and the organic layer was separated. The aqueous phase was extracted with DCM (30 mL*3). The combined organic layers were washed with brine, dried over anhydrous sodium sulfate and concentrated to dryness under vaccum to give the title compound (5 g, 79%, yield: 82%) as red oil. LCMS (ESI) m/z calcd for C₂₁₁H₃₅NO₂: 333; found: 334 (M+1). ¹H NMR (400 MHz, Chloroform-d) δ 7.69 (s, 2H), 7.30-7.26 (m, 3H), 7.23-7.14 (m, 2H), 4.17 (t, J=6.8 Hz, 1H), 4.04 (t, J=6.8 Hz, 2H), 3.29-3.15 (m, 2H), 1.54-1.46 (m, 2H), 1.33-1.25 (m, 18H), 0.88 (t, J=6.8 Hz, 3H).

### Step 3: Dodecyl (chloro(phenoxy)phosphoryl)-L-phenylalaninate

To a solution of dodecyl L-phenylalaninate (3.9 g, 11.69 mmol) and triethylamine (2.367 g, 23.39 mmol) in DCM (400 mL) stirred under nitrogen at 0° C. was added. phenyl phosphorodichloridate (2.467 g, 11.69 mmol) dropwise. The reaction mixture was stirred at 25° C. for 1 hour. LCMS indicated completion of. reaction. The reaction mixture was concentrated and the crude residue was purified by flash column chromatography (silica gel, 40 g, pet. ether:EtOAc=5:1) to give the title compound (1.06 g, 91%, yield: 16.24%) as a white solid. LCMS (ESI) m/z calcd for C₂₇H₃₉ClNO₄P: 507; found: 508 (M+1). ¹H NMR (400 MHz, Chloroform-d) δ 7.37 (t, J=7.6 Hz, 2H), 7.32-7.26 (m, 3H), 7.26-7.06 (m, 5H), 4.49-4.33 (m, 1H), 4.18-3.92 (m, 3H), 3.21-3.06 (m, 2H), 1.64-1.50 (m, 2H), 1.26 (d, J=2.4 Hz, 18H), 0.88 (t, J=6.8 Hz, 3H).

### Step 4: Dodecyl ((((2R,3S,5R)-2-ethynyl-5-(2-fluoro-6-(((4-methoxyphenyl)diphenylmethyl) amino)-9H-purin-9-yl)-3-(4-methoxyphenyl)diphenylmethoxy)tetrahydrofuran-2-yl)methoxy)(phenoxy)phosphoryl)-L-phenylalaninate

To a solution of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (2.309 g, 2.76 mmol) in pyridine (10 mL)/THF (10 mL) stirred under nitrogen at 25° C. was added a solution of 1M tert-butylmagnesium chloride/THF (3.86 mL, 3.86 mmol) dropwise. Then, to the resulting reaction mixture was added solution of dodecyl (chloro(phenoxy)phosphoryl)-L-phenylalaninate (1.4 g, 2.76 mmol) in THF (5 mL) dropwise. The reaction mixture was stirred at 25° C. for 1.5 hours. LCMS indicated completion of. reaction. The reaction was concentrated to dryness in vaccum. The crude product was subjected to reverse phase-HPLC purification (C18, 25-100% MeCN/water with 0.1% HN₄HCO₃) to give the title compound (1.6 g, 99%, yield: 43.9%) as yellow oil. LCMS (ESI) m/z calcd for C₇₉H₈₂FN₆O₉P: 1309; found: 1310 (M+1). ¹H NMR (400 MHz, Chloroform-d) δ 7.72-7.26 (m, 12H), 7.26-6.90 (m, 21H), 6.83-6.70 (m, 4H), 6.15-6.11 (m, 1H), 4.51 (q, J=6.4 Hz, 1H), 4.29-4.17 (m, 1H), 4.09-4.05 (m, 2H), 3.94-3.92 (m, 2H), 3.85-3.83 (m, 1H), 3.77 (s, 3H), 3.72 (s, 3H), 3.30 (t, J=10.4 Hz, 1H), 2.99-2.77 (m, 3H), 1.94-1.92 (m, 1H), 1.84-1.72 (m, 1H), 1.65 (d, J=7.2 Hz, 2H), 1.46 (d, J=7.6 Hz, 2H), 1.27-1.22 (m, 18H), 0.88 (t, J=6.8 Hz, 3H).

### Step 5: Dodecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

To a solution of. dodecyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran yl)methoxy)(phenoxy)phosphoryl)-L-phenylalaninate (1.5 g, 1.145 mmol) in DCM (15.0 mL) stirred at room 25° C. was added TFA (1.5 mL) dropwise. The reaction mixture was stirred at 25° C. for 2 hours. LCMS indicated completion of. reaction. The reaction was quenched with saturated aqueous NaHCO₃ and pH was adjusted to 8, The organic layer was separated, dried over anhydrous sodium sulfate and concentrated to dryness under vacuum. The crude product was purified by flash column chromatography (silica gel, 40 g, pet. ether:EtOAc=1:99) to give the title compound (720 mg) as yellow solid. The diastereomers were separated with Prep-SFC (Column: CHIRALPAK IH, 2.0*25 cm, 5 μm; Mobile Phase A:CO₂, Mobile Phase B:MeOH-Preparative; Flow rate: 40 mL/min; Gradient: 40% B; 254 nm; RT₂: 3.28; RT₂: 5.52; Injection Volumn: 4.5 mL; Number of Runs: 8; to give first eluting isomer (Example 3A, RT1: 3.28 min, 275.7 mg, 99.47%, yield: 31.3%) as an off-white solid; LCMS (ESI) m/z calcd for C₃₉H₅₀FN₆O₇P: 764; found: 765 (M+1); ¹H NMR (400 MHz, Chloroform-d) 7.96 (s, 1H), 7.28-7.26 (m, 1H), 7.24-7.10 (m, 9H), 6.33-6.10 (m, 3H), 4.71 (t, J=8 Hz, 1H), 4.30-4.18 (m, 3H), 4.04-3.97 (m, 3H), 3.75 (br s, 1H), 2.99 (d, 2H), 2.73-2.68 (m, 3H), 1.53-1.50 (m, 1H), 1.31-1.23 (m, 18H), 0.88 (t, J=6.4 Hz, 3H); and second eluting isomer (Example 3B, RT2: 5.52 min, 313.5 mg, 99.60%, yield: 35.6%) as yellow solid; LCMS (ESI) m/z calcd for C₃₉H₅₀FN₆O₇P: 764; found: 765 (M+1); ¹H NMR (400 MHz, Chloroform-d) 7.82 (s, 1H), 7.31-7.26 (m, 2H), 7.24-7.22 (m, 3H), 7.17-7.13 (m, 3H), 7.08-7.06 (m, 2H), 6.33-6.31 (m, 1H), 6.17 (br s, 2H), 4.65-4.60 (m, 1H), 4.21-4.15 (m, 2H), 4.04 (t, J=6.4 Hz, 1H), 3.98-3.95 (m, 1H), 3.70-3.68 (m, 2H), 3.00-2.96 (m, 2H), 2.71 (s, 1H), 2.70-2.50 (m, 2H), 1.60-1.50 (m, 2H), 1.30-1.24 (m, 18H), 0.88 (t, J=6.8 Hz, 3H).

### Example 4

### Decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

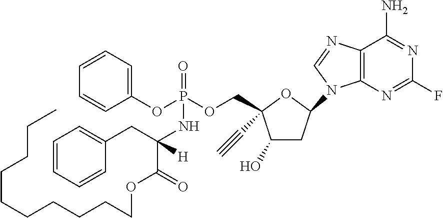 [SMILES: C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1O]

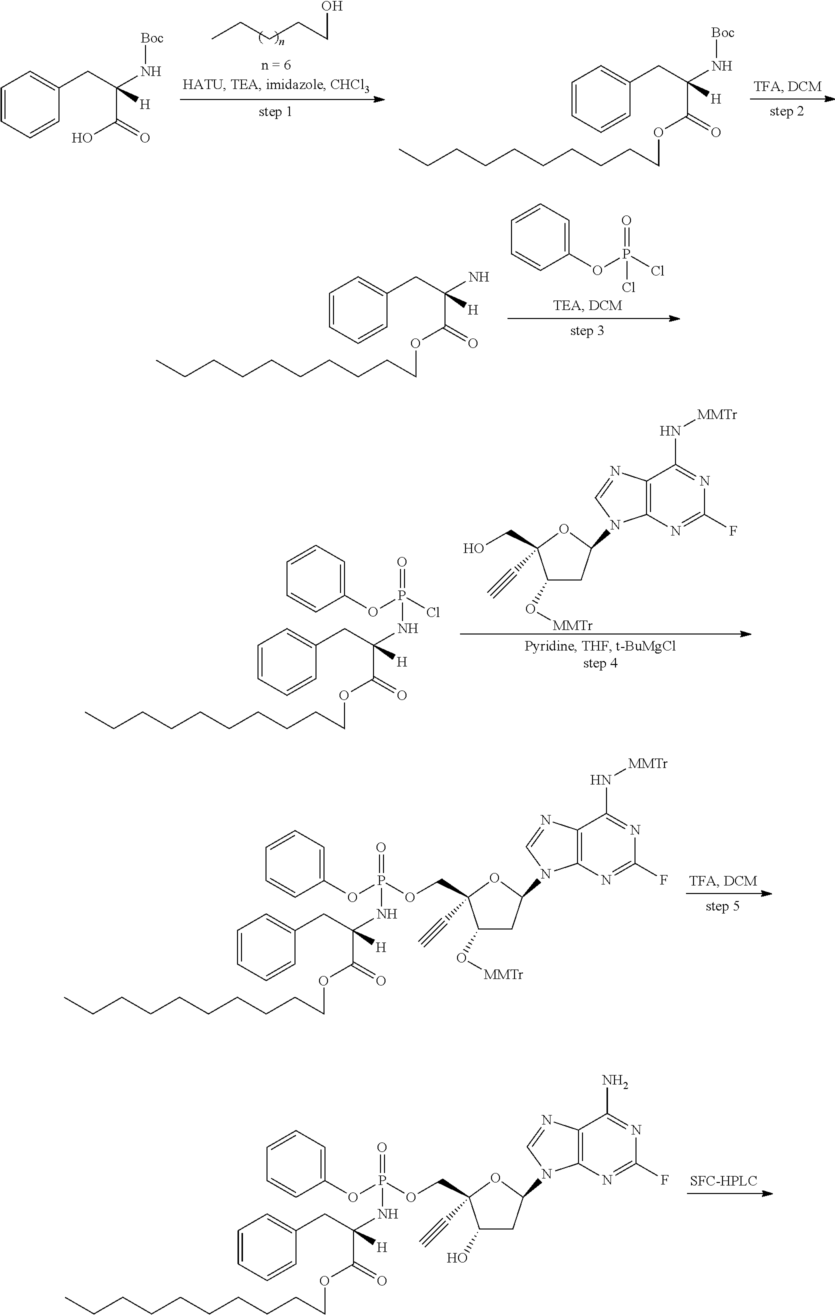

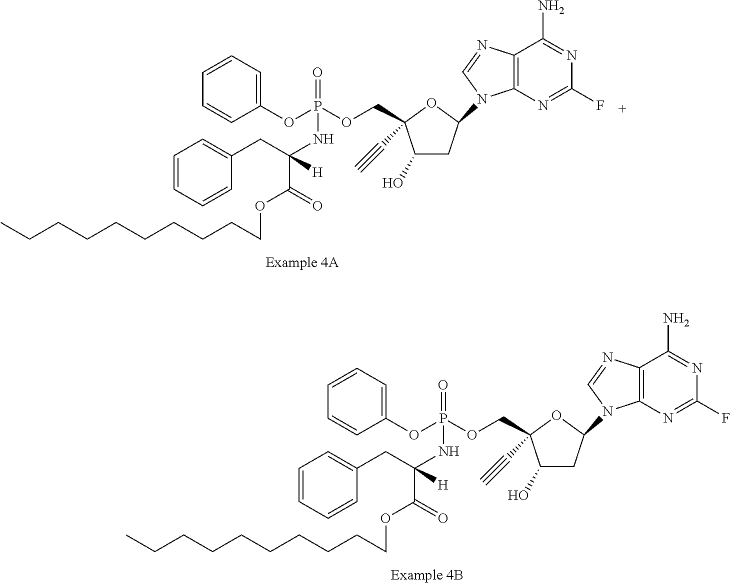 [SMILES: C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OCCCCCCCCCC)Oc2ccccc2)O[C@@H](n2cnc3c(N)nc(F)nc32)C[C@@H]1O]

### Step 1: Decyl (tert-butoxycarbonyl)-L-phenylalaninate

To a stirred mixture of (tert-butoxycarbonyl)-L-phenylalanine (10 g, 37.7 mmol) in chloroform (100 mL) was added HATU (21.50 g, 56.5 mmol), decan-1-ol (8.95 g, 56.5 mmol), TEA (15.76 mL, 113 mmol) and imidazole (7.70 g, 113 mmol) at 0° C. The reaction mixture was stirred for 5 h at room temperature. LCMS indicated completion of. reaction. The reaction was diluted with water (100 mL), extracted with DCM (2×100 mL) and the combined organic extracts were washed with saturated aqueous NaHCO₃ (2×100 mL) and brine (50 mL). The mixture was concentrated to dryness under vacuum. The residue was purified by silica gel column (330 g, pet. ether:EtOAc=2:1) to afford decyl (tert-butoxycarbonyl)-L-phenylalaninate (9.1 g, 22.44 mmol, 59.5% yield) as yellow oil. LCMS (ESI) m/z calcd for C₂₄H₃₉NO₄: 405; found: 305 (M−100).

### Step 2: Decyl L-phenylalaninate

To a stirred mixture of decyl (tert-butoxycarbonyl)-L-phenylalaninate (7.0 g, 17.26 mmol) in DCM (80 mL) was added TFA (15 mL, 195 mmol) at 0° C. The reaction mixture was stirred for 1 h at room temperature. LCMS indicated completion of. reaction. The reaction mixture was quenched with saturated aqueous NaHCO₃ (30 mL). Organic layer separated and aqueous layer was extracted with DCM (20 mL*3). The combined organic layer dried and concentrated to dryness under vacuum to afford decyl L-phenylalaninate (5.6 g, 18.33 mmol, 106% yield) as yellow oil. LCMS (ESI) m/z calcd for C₁₉H₃₁NO₂: 305; found: 306 (M+1). ¹H NMR (400 MHz, CDCl₃) δ 7.31-7.17 (m, 5H), 4.07 (t, J=8 Hz, 2H), 3.80 (t, J=4 Hz, 1H), 3.63 (s, 2H), 3.12-3.07 (m, 1H), 2.96-2.91 (m, 1H), 1.61-1.54 (m, 2H), 1.32-1.26 (m, 14H), 0.88 (t, J=4 Hz, 3H).

### Step 3: Decyl (chloro(phenoxy)phosphoryl)-L-phenylalaninate

To a stirred mixture of decyl L-phenylalaninate (2.0 g, 6.55 mmol) in DCM (20 mL) was added TEA (2.74 mL, 19.64 mmol) and decyl (chloro(phenoxy)phosphoryl)-L-phenylalaninate at 0° C. The reaction mixture was stirred for 1 h at room temperature. LCMS indicated completion of. reaction. The reaction mixture was used in the next step directly without further purification. LCMS (ESI) m/z calcd for C₂₅H₃₅ClNO₄P: 479; found: 480 (m+1).

### Step 4: Decyl ((((2R,3S,5R)-2-ethynyl-5-(2-fluoro-6-(((4-methoxyphenyl)diphenylmethyl)amino)-9H-purin-9-yl)-3-(4-methoxyphenyl)diphenylmethoxy)tetrahydrofuran-2-yl)methoxy)(phenoxy)phosphoryl)-L-phenylalaninate

To a solution of ((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methanol (3.5 g, 4.18 mmol). in pyridine/THF stirred under nitrogen at room temperature was added a solution of tert-butylmagnesium chloride/THF (5.85 ml, 5.85 mmol). The reaction mixture was stirred at 25° C. for 0.5 hour. Then the decyl (chloro(phenoxy)phosphoryl)-L-phenylalaninate (2.005 g, 4.18 mmol) was added to the reaction at 25° C. The resulting mixture was stirred at room temperature for 6 hour. LCMS indicated completion of. reaction. The reaction mixture was quenched with water (50 mL), extracted with DCM (3*50 mL). The combined organic phases were dried and evaporated under vacuum to give the crude product as a red oil. The residue was purified by silica gel column (120 g, pet. ether:EtOAc=1:1) to afford decyl ((((2R,3S,5R)-2-ethynyl-5-(2-fluoro-6-(((4-methoxy-phenyl)diphenylmethyl)amino)-9H-purin-9-yl)-3-((4-methoxyphenyl)diphenylmethoxy)tetrahydrofuran-2-yl)methoxy)(phenoxy)phosphoryl)-L-phenylalaninate (3.88 g, 2.180 mmol, 52.2% yield). as a yellow solid. LCMS (ESI) m/z calcd for C₇₇H₇₈FN₆O₉P: 1281; found: 1282 (m+1).

### Step 5: Decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred mixture of decyl ((((2R,3S,5R)-2-ethynyl-5-(2-fluoro-6-(((4-methoxyphenyl)di-phenyl-methyl)amino)-9H-purin-9-yl)-3-((4-methoxyphenyl)diphenylmethoxy)tetrahydrofuran-2-yl)methoxy)(phenoxy)phosphoryl)-L-phenylalaninate (3.0 g, 2.341 mmol) in DCM (40 mL) was added TFA (2.0 mL) at room temperature. The reaction mixture was stirred for 3 h at room temperature. LCMS showed presence of desired product. The pH was adjusted to 6-7 with saturated aqueous NaHCO₃, extracted with DCM (3*10 mL). The combined organic layers were washed with brine and dried over Na2SO4. After filtration, the filtrate was concentrated to dryness under vacuum. The residue was purified by silica gel column (80 g, EtOAc) to afford decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1.2 g) as a yellow oil. The diastereomers were separated by following condition: Column: CHIRALPAK IH, 2.0*25 cm, 5 μm; Mobile Phase A: CO2, Mobile Phase B: MeOH; Preparative; Flow rate: 40 mL/min; Gradient: 40% B; 254 nm; RT1:3.08; RT2:5.49; Injection Volumn: 3.5 mL; number of runs: 9; the collected fractions were concentrated to dryness in vacuum to first eluting isomer (Example 4A, RT1: 3.08 min) which was re-crystallized from MeOH/H₂O in the ratio of 1:1. The solid was collected by filtration and dried under sun lamp (45° C.) to afford 210 mg, (0.273 mmol, 11.66% yield) as a white amorphous solid; LCMS (ESI) m/z calcd for C₃₇H₄₆FN₆O₇P: 736; found: 737 (m+1). ¹H NMR (400 MHz, CDCl₃) δ 7.96 (s, 1H), 7.28-7.24 (m, 1H), 7.23-7.17 (m, 4H), 7.14-7.08 (m, 5H), 6.33-6.30 (m, 1H), 6.25-6.00 (m, 1H), 4.71 (t, J=8 Hz, 1H), 4.29-4.18 (m, 3H), 4.03 (t, J=8 Hz, 2H), 3.93 (t, J=12 Hz, 1H), 2.99 (d, J=4 Hz, 2H), 2.73-2.68 (m, 3H), 1.52 (t, J=8 Hz, 2H), 1.27 (t, J=12 Hz, 14H), 0.86 (t, J=8 Hz, 3H); and the second eluting isomer (Example 4B, RT2: 5.49 min) was re-crystallized from MeOH. The solid was collected by filtration and dried under sun lamp (45° C.) to afford 222 mg (0.298 mmol, 12.73% yield) as white crystal solid. LCMS (ESI) m/z calcd for C₃₇H₄₆FN₆O₇P: 736; found: 737 (m+1). ¹H NMR (400 MHz, CDCl₃) δ 7.82 (s, 1H), 7.30-7.22 (m, 5H), 7.14 (t, J=8 Hz, 3H), 7.07 (d, J=4 Hz, 2H), 6.33-6.03 (m, 3H), 4.63 (s, 1H), 4.18 (t, J=12 Hz, 2H), 4.05-3.94 (m, 3H), 3.72 (d, J=4 Hz, 1H), 3.00-2.96 (m, 2H), 2.71-2.59 (m, 3H), 1.54 (s, 2H), 1.27 (d, J=12 Hz, 14H), 0.89-0.86 (m, 3H).

### Example 5

### Octyl ((((2R,3S,5R)-5-(6-amino-2-fluoro-9H-purin-9-yl)-2-ethynyl-3-hydroxytetrahydrofuran-2-yl)methoxy)(2-(octyloxy)-2-oxoethoxy)phosphoryl)-L-phenylalaninate

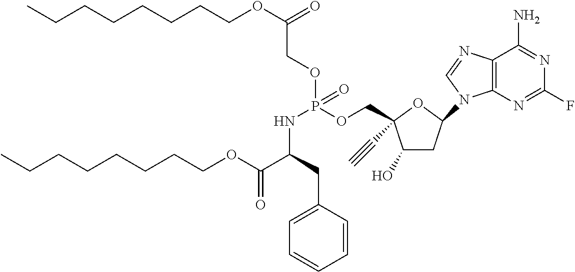 [SMILES: C#C[C@]1(COP(=O)(N[C@@H](Cc2ccccc2)C(=O)OCCCCCCCC)OCC(=O)OCCCCCCCC)O[C@@H](n2cnc3c(N)nc(F)nc32)C[C@@H]1O]

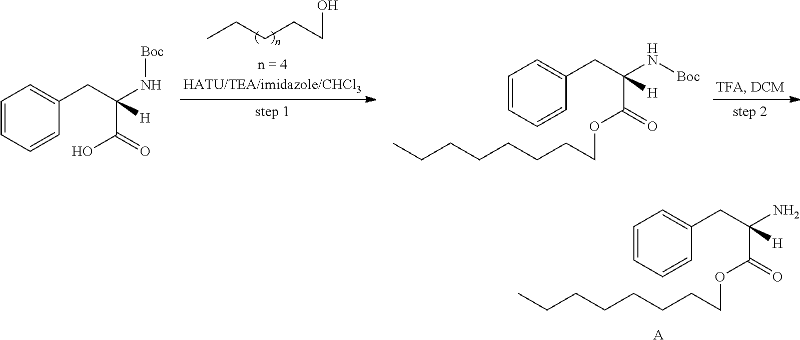

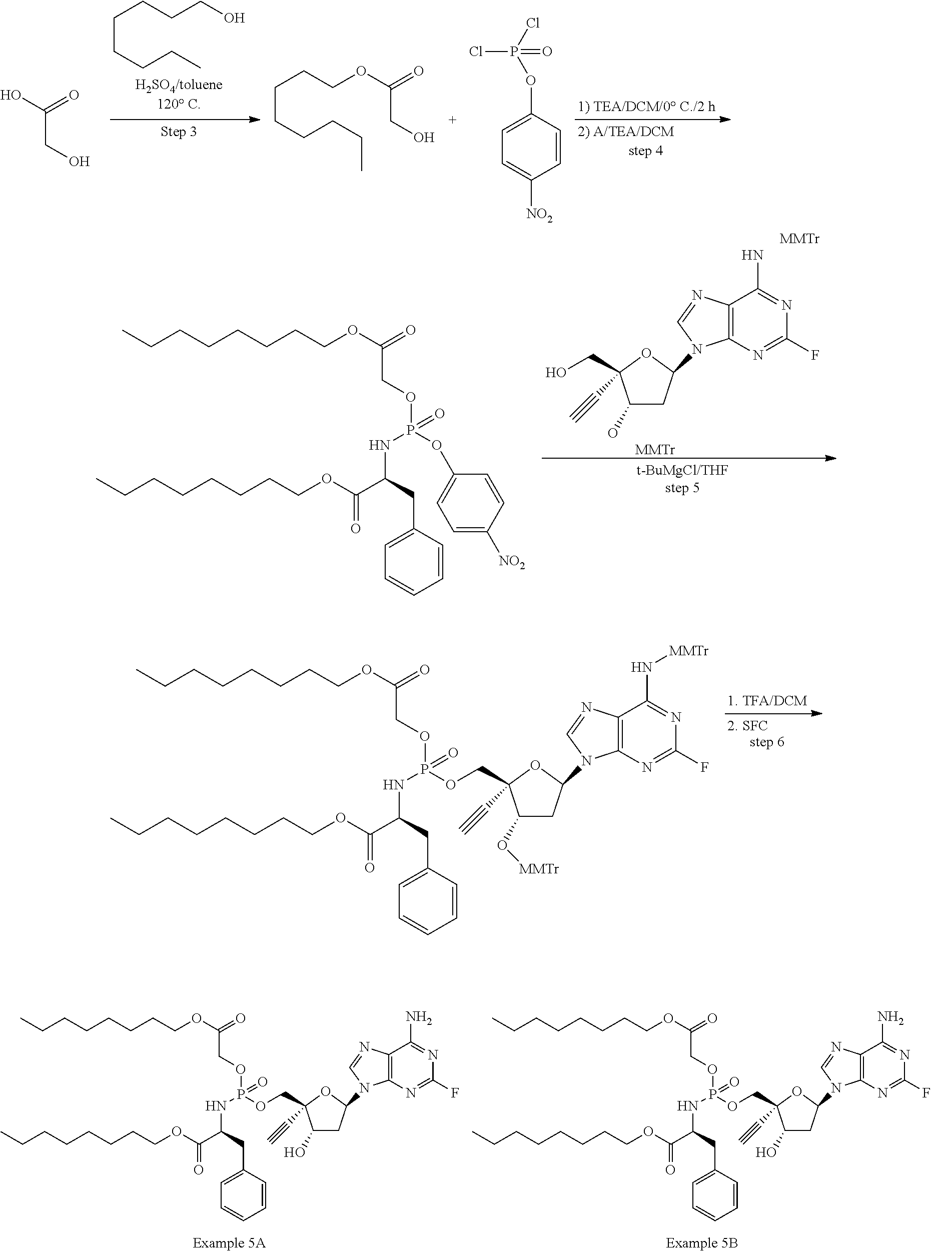

### Step 1: Octyl (tert-butoxycarbonyl)-L-phenylalaninate

To a stirred solution of (tert-butoxycarbonyl)-L-phenylalanine (15 g, 56.5 mmol) in chloroform (100 mL) was added HATU (32.2 g, 85 mmol), octan-1-ol (11.04 g, 85 mmol), TEA (23.64 mL, 170 mmol) and imidazole (11.55 g, 170 mmol) at 0° C. The reaction mixture was stirred for 16 h at room temperature. LCMS indicated completion of. reaction. The reaction mixture was quenched with water (100 mL) and extracted with DCM (100 mL*2). The organic phases were combined, washed with brine (30 mL), dried over Na2SO4 and concentrated under vacuum. The residue was purified by flash column chromatography (silica gel, 330 g, pet. ether:EtOAc=10:1) to give octyl (tert-butoxycarbonyl)-L-phenylalaninate (13 g, 95%, 57.9% yield) as yellow oil. LCMS (ESI) m/z calcd for C₂₂H₃₅NO₄: 377; found: 278 (M−99). ¹H NMR (400 MHz, Chloroform-d) δ 7.31-7.21 (m, 3H), 7.15-7.11 (m, 2H), 4.98 (d, J=8.4 Hz, 1H), 4.60-4.53 (m, 1H), 4.11-4.03 (m, 2H), 3.13-3.00 (m, 2H), 1.61-1.52 (m, 2H), 1.46-1.35 (m, 9H), 1.32-1.20 (m, 10H), 0.91-0.86 (m, 3H).

### Step 2: Octyl L-phenylalaninate

To a stirred mixture of octyl (tert-butoxycarbonyl)-L-phenylalaninate (13.0 g, 34.4 mmol) in DCM (80 mL) was added TFA (15 mL, 195 mmol) at 0° C. The reaction mixture was stirred for 1 h at room temperature. LCMS indicated completion of. reaction. The reaction mixture was quenched with NaHCO₃ (100 mL). and extracted with DCM (80 mL×2). The organic phases were combined, washed with brine (15 mL), dried over Na₂SO₄ and concentrated under vacuum to give octyl L-phenylalaninate (8 g, 90%, 71.2% yield) as white solid. LCMS (ESI) m/z calcd for C₁₇H₂₇NO₂: 277; found: 278 (M+H). ¹H NMR (400 MHz, Chloroform-d) δ 7.33-7.16 (m, 5H), 4.08 (t, J=6.4 Hz, 2H), 3.81 (dd, J=5.6, 7.6 Hz, 1H), 3.46 (br, s, 2H), 3.10 (dd, J=5.6, 13.6 Hz, 1H), 3.10 (dd, J=7.6, 13.6 Hz, 1H), 1.63-1.52 (m, 2H), 1.35-1.17 (m, 10H), 0.88 (t, J=6.8 Hz, 3H).

### Step 3: Octyl 2-hydroxyacetate

To a mixture of 2-hydroxyacetic acid (6 g, 79 mmol) and octan-1-ol (20.55 g, 158 mmol) in toluene (30 mL) was added H₂SO₄ (1 mL, 18.76 mmol). The mixture was stirred for 4 h at 120° C. with a Dean-stark apparatus and cooled to room temperature. The solvent was removed under vacuum and the residue was purified by gel silica column (120 g, pet. ether:EtOAc=8:1) to give octyl 2-hydroxyacetate (12 g, 85%, 64.6% yield) as a colorless oil. ¹H NMR (400 MHz, Chloroform-d) δ 4.27-4.08 (m, 4H), 2.43 (br s, 1H), 1.74-1.57 (m, 2H), 1.50-1.16 (m, 10H), 0.97-0.80 (m, 3H).

### Step 4: Octyl ((4-nitrophenoxy)(2-(octyloxy)-2-oxoethoxy)phosphoryl)-L-phenylalaninate

To a mixture of 4-nitrophenyl phosphorodichloridate (2.99 g, 11.69 mmol) and octyl 2-hydroxyacetate (2.2 g, 11.69 mmol) in DCM (26 mL) was added TEA (0.81 mL, 5.85 mmol) dropwise at 20° C. under N₂ atmosphere. The reaction was stirred for 2 h at 20° C. Then, octyl L-phenylalaninate (3.24 g, 11.69 mmol) in DCM (10 mL) and TEA (1.52 mL, 11.70 mmol) was added. The reaction mixture was stirred for another 1 h at 20° C. LCMS indicated completion of. reaction. The majority of the solvent was removed under vacuum and the residue was purified by gel silica column (120 g, pet. ether; etOAc=5:1) to give octyl ((4-nitrophenoxy)(2-(octyloxy)-2-oxoethoxy)phosphoryl)-L-phenylalaninate (1.8 g, 86%, 19.00% yield) as white semisolid. LCMS (ESI) m/z calcd for C₃₃H₄₉N₂O₉P: 648; found: 649 (M+H). ¹H NMR (400 MHz, Chloroform-d) δ 8.21-8.09 (m, 2H), 7.32-7.18 (m, 5H), 7.16-7.09 (m, 2H), 4.50-4.26 (m, 3H), 4.18-4.04 (m, 4H), 3.75-3.64 (m, 1H), 3.15-2.90 (m, 2H), 1.67-1.52 (m, 4H), 1.36-1.19 (m, 20H), 0.91-0.81 (m, 6H).

### Step 5: Octyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran yl)methoxy)(2-(octyloxy)-2-oxoethoxy)phosphoryl)-L-phenylalaninate

To a mixture of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (1.2 g, 1.432 mmol) in THF (12 mL) was added tert-butylmagnesium chloride (2.148 mL, 2.148 mmol) at 20° C. under N₂ atmosphere. The reaction mixture was stirred for 1 h at room temperature. Then octyl ((4-nitrophenoxy)(2-(octyloxy)-2-oxoethoxy)phosphoryl)-L-phenylalaninate (1.858 g, 2.86 mmol) was added to this mixture. The mixture was stirred for 12 h at room temperature. LCMS indicated completion of. reaction. The reaction was concentrated and the residue was purified by reverse phase column with the following conditions: column C18 silica gel 120 g, Mobile phase A: water (10 mmol/L NH₄HCO₃) and B: CAN (30% to 100% ACN in 20 min), Flow rate: 80 ml/min). to give octyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(2-(octyloxy)-2-oxoethoxy)phosphoryl)-L-phenylalaninate (900 mg, 97%, 45.2% yield) as white semisolid. LCMS (ESI) m/z calcd for C₇₉H₈₈FN₆O₁₁P: 1346; found: 1347 (M+1). ¹H NMR (400 MHz, Chloroform-d) δ 7.86 (s, 1H), 7.55-7.49 (m, 4H), 7.41-7.06 (m, 26H), 6.82-6.75 (m, 4H), 6.20-6.12 (m, 1H), 4.57-4.46 (m, 1H), 4.28-3.91 (m, 9H), 3.88-3.71 (m, 6H), 3.52-3.21 (m, 3H), 2.96-2.79 (m, 3H), 1.86-1.44 (m, 8H), 1.38-1.16 (m, 16H), 0.91-0.78 (m, 6H).

### Step 6: Octyl ((((2R,3S,5R)-5-(6-amino-2-fluoro-9H-purin-9-yl)-2-ethynyl-3-hydroxytetrahydrofuran-2-yl)methoxy)(2-(octyloxy)-2-oxoethoxy)phosphoryl)-L-phenylalaninate

To a solution of octyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)meth-oxy)(2-(octyloxy)-2-oxoethoxy)phosphoryl)-L-phenylalaninate (1.3 g, 0.965 mmol) in DCM (15 mL) was added TFA (2 mL, 26 mmol) dropwise at room temperature and stirred for 30 min. LCMS indicated completion of. reaction. The reaction mixture was quenched with NaHCO₃ (100 mL), extracted with DCM (60 mL×2). The organic phases were combined, washed with brine (15 mL), dried over Na2SO4, and concentrated under vacuum. The residue was purified by silica gel column (120 g, pet. ether:EtOAc=1:1) to give the title compound as colorless semisolid. The diastereomers were separated by Prep-SFC-HPLC. with the following conditions: Column: CHIRALPAK IH, 2.0*25 cm, 5 μm; Mobile Phase A: CO2, Mobile Phase B: MeOH-Preparative; Flow rate: 40 mL/min; Gradient: 30% B; 220 nm; RT1: 3.87; RT2: 5.2; Injection Volumn: 4 mL; number of runs: 9; to give first eluting isomer (Example 5A, RT1: 3.87, 232.0 mg, 95.7% purity, 28.7% yield) as white solid. LCMS (ESI) m/z calcd for C₃₉H₅₆FN₆O₉P: 802; found: 803 (M+1). ¹H NMR (400 MHz, Chloroform-d) δ 8.11 (s, 1H), 7.27-7.16 (m, 5H), 6.36 (dd, J=4.4 Hz, 7.2 Hz, 2H), 4.70 (t, J=7.6 Hz, 1H), 4.45-4.38 (m, 1H), 4.24-4.05 (m, 7H), 3.81 (t, J=10.8 Hz, 1H), 3.72 (dd, J=7.2 Hz, 14.0 Hz, 1H), 3.14-3.07 (m, 1H), 2.92 (dd, J=8.0 Hz, 14.0 Hz, 1H), 2.77-2.64 (m, 3H), 1.66-1.51 (m, 4H), 1.35-1.18 (m, 20H), 0.90-0.84 (m, 6H); and the second eluting isomer (Example 5B, RT2: 5.2, 224.6 mg, 97.5% purity, 28.3 yield) as colorless semisolid. LCMS (ESI) m/z calcd for C₃₉H₅₆FN₆O₉P: 802; found: 803 (M+1). ¹H NMR (400 MHz, DMSO) δ 8.22 (s, 1H), 7.83 (br, s, 2H), 7.26-7.11 (m, 5H), 6.26 (dd, J=5.2 Hz, 6.8 Hz, 1H), 5.76 (dd, J=10.4 Hz, 12.8 Hz, 2H), 4.54-4.47 (m, 1H), 4.15-3.84 (m, 9H), 3.63 (s, 1H), 2.96-2.88 (m, 1H), 2.83-2.63 (m, 2H), 2.45-2.37 (m, 1H), 1.54-1.37 (m, 4H), 1.28-1.08 (m, 20H), 0.87-0.78 (m, 6H).

### Example 6

### Octyl ((((2R,3S,5R)-5-(6-amino-2-fluoro-9H-purin-9-yl)-2-ethynyl-3-hydroxytetrahydrofuran-2-yl)methoxy)(((S)-1-(octyloxy)-1-oxopropan-2-yl)oxy)phosphoryl)-L-phenylalaninate

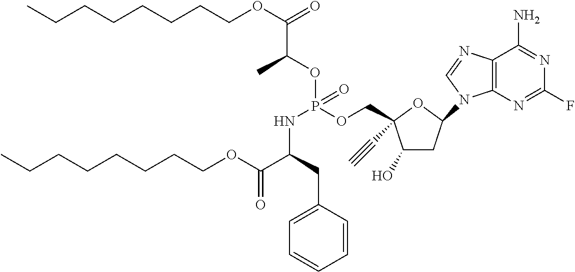 [SMILES: C#C[C@]1(COP(=O)(N[C@@H](Cc2ccccc2)C(=O)OCCCCCCCC)O[C@@H](C)C(=O)OCCCCCCCC)O[C@@H](n2cnc3c(N)nc(F)nc32)C[C@@H]1O]

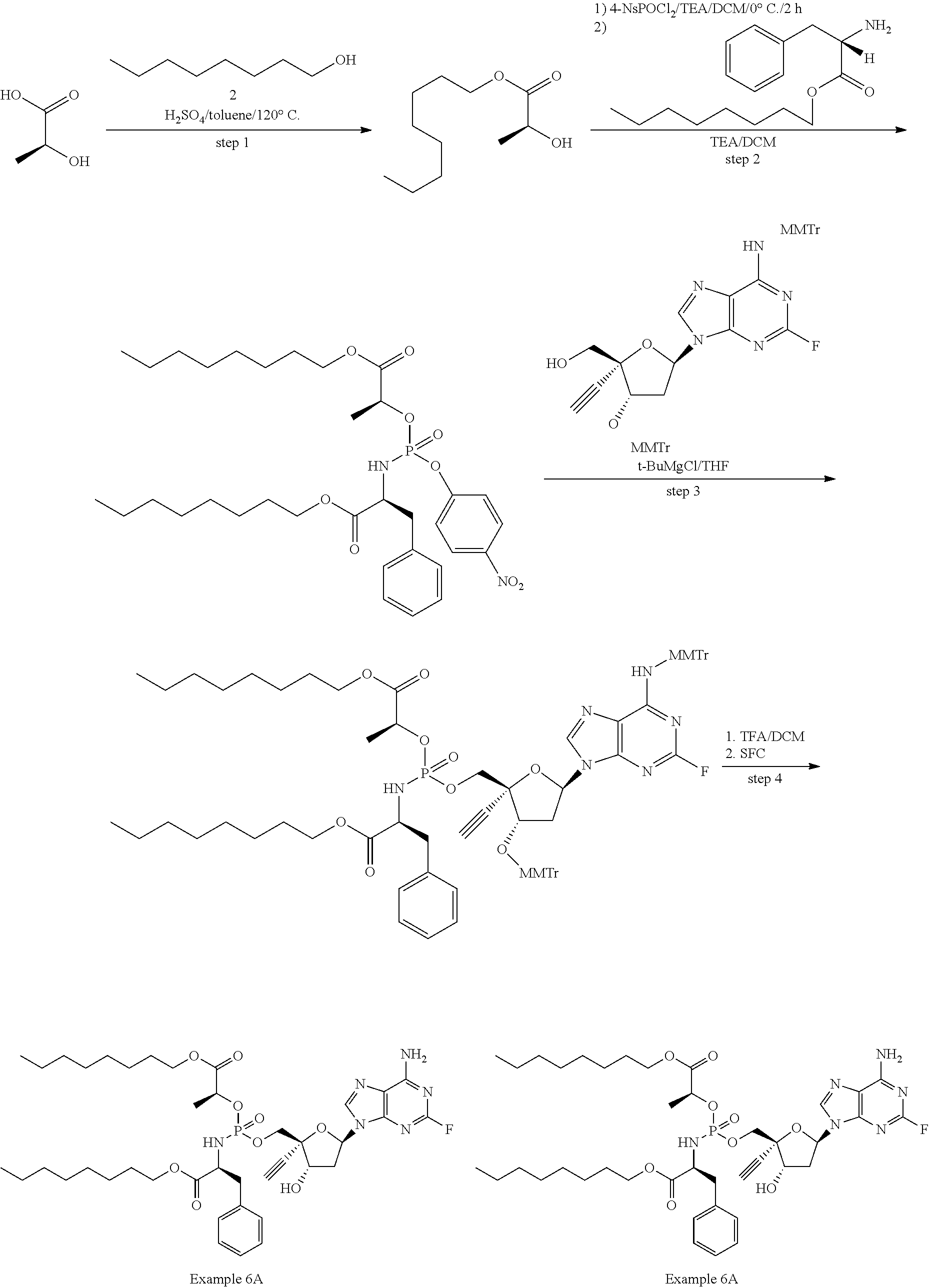

### Step 1: octyl (S)-2-hydroxypropanoate

To a mixture of (S)-2-hydroxypropanoic acid (3 g, 33.3 mmol) and octan-1-ol (8.67 g, 66.6 mmol) in toluene (30 mL) was added H₂SO₄ (1 mL, 18.76 mmol). The mixture was stirred for 4 h at 120° C. with a Dean-Stark apparatus and cooled to room temperature. The solvent was removed under vacuum and the residue was purified by gel silica column (120 g, pet. ether:EtOAc=8:1) to give octyl (S)-2-hydroxypropanoate (2.5 g, 11.74 mmol, 35.3 yield) as colorless oil. ¹H NMR (400 MHz, Chloroform-d) δ 4.28-4.11 (m, 3H), 2.57 (br s, 1H), 1.70-1.57 (m, 2H), 1.40 (d, J=7.2 Hz, 3H), 1.37-1.20 (m, 10H), 0.90-0.84 (m, 3H).

### Step 2: Octyl((4-nitrophenoxy)(((S)-1-(octyloxy)-1-oxopropan-2-yl)oxy)phosphoryl)-L-phenylalaninate

To a mixture of 4-nitrophenyl phosphorodichloridate (3.16 g, 12.36 mmol) and octyl (S)-2-hydroxypropanoate (2.5 g, 12.36 mmol) in DCM (26 mL) was added TEA (0.81 mL, 5.85 mmol) dropwise at 0° C. under N₂ atmosphere. The reaction was stirred for 2 h at 0° C. Then octyl L-phenylalaninate (3.43 g, 12.36 mmol) in DCM (10 mL) and TEA (1.52 mL, 11.70 mmol) was added at 0° C. The reaction mixture was stirred for another 1 h at 0° C. LCMS indicated completion of. reaction. The most of the solvent was removed under vacuum and the residue was purified by gel silica column (120 g, EA: pet. ether=5:1) to give octyl ((4-nitrophenoxy) (((S)-1-(octyloxy)-1-oxopropan-2-yl)oxy)phosphoryl)-L-phenylalaninate (2.5 g, 89%, 27.2% yield) as pale yellow oil. LCMS (ESI) m/z calcd for C₃₄H₅₁N₂O₉P: 662; found: 663 (M+H). ¹H NMR (400 MHz, Chloroform-d) δ 8.22-8.12 (m, 2H), 7.34-7.18 (m, 5H), 7.15-7.06 (m, 2H), 4.92-4.64 (m, 1H), 4.43-4.24 (m, 1H), 4.18-3.98 (m, 4H), 3.68-3.61 (m, 1H), 3.14-2.95 (m, 2H), 1.68-1.44 (m, 7H), 1.39-1.16 (m, 20H). 0.91-0.81 (m, 6H).

### Step 3: octyl octyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methoxy) (((S)-1-(octyloxy)-1-oxopropan-2-yl)oxy)phosphoryl)-L-phenylalaninate

To a mixture of ((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methanol (1.6 g, 1.909 mmol) in THF (16 mL) was added tert-butylmagnesium chloride/THF (2.86 mL, 2.86 mmol) at 20° C. under N₂. The reaction mixture was stirred for 1 h at room temperature. Then, octyl ((4-nitrophenoxy)(((S)-1-(octyloxy)-1-oxopropan-2-yl)oxy)phosphoryl)-L-phenylalaninate (2.53 g, 3.82 mmol) was added to the mixture. The mixture was stirred for 12 h at room temperature. LCMS indicated completion of. reaction. The reaction was concentrated and residue was purified by reverse phase column with the following conditions: column C18 silica gel 120 g, Mobile phase A: water (10 mmol/L NH₄CO₃) and B: CAN (30% to 100% ACN in 20 min), Flow rate: 80 mL/min. to give octyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy) (((S)-1-(octyloxy)-1-oxopropan-2-yl)oxy)phosphoryl)-L-phenylalaninate (750 mg, 98%, 28.3 yield) as white semisolid. LCMS (ESI) m/z calcd for C₈₀H₉₀FN₆O₁₁P: 1360; found: 1361 (M+1). ¹H NMR (400 MHz, Chloroform-d) δ 7.94 (s, 1H), 7.55-7.48 (m, 4H), 7.41-7.04 (m, 26H), 6.83-6.71 (m, 4H), 6.18-6.07 (m, 1H), 4.76-4.37 (m, 3H), 4.27-3.88 (m, 8H), 3.81-3.70 (m, 6H), 3.23 (t, J=10.4 Hz, 1H), 2.98-2.75 (m, 3H), 1.91-1.43 (m, 8H), 1.40-1.36 (m, 3H), 1.34-1.14 (m, 16H), 0.91-0.80 (m, 6H).

### Step 4: Octyl octyl ((((2R,3S,5R)-5-(6-amino-2-fluoro-9H-purin-9-yl)-2-ethynyl-3-hydroxytetrahydrofuran-2-yl)methoxy)(((S)-1-(octyloxy)-1-oxopropan-2-yl)oxy)phosphoryl)-L-phenylalaninate

To a solution of octyl octyl ((((2R,3S,5R)-2-ethynyl-5-(2-fluoro-6-(((4-methoxyphenyl)-diphenylmethyl)amino)-9H-purin-9-yl)-3-((4-methoxyphenyl)diphenylmethoxy)tetrahydrofuran-2-yl)methoxy)(((S)-1-(octyloxy)-1-oxopropan-2-yl)oxy)phosphoryl)-L-phenylalaninate (700 mg, 0.514 mmol) in DCM (10 mL) was added TFA (1.5 mL, 19.47 mmol) dropwise at room temperature and stirred for 30 min. LCMS indicated completion of. reaction. The reaction mixture was quenched with NaHCO₃ (80 mL) and extracted with DCM (60 mL×2). The organic phases were combined, washed with brine (15 mL), dried over Na2SO4, and concentrated under vacuum. The residue was purified by silica gel column chromatography (120 g, pet. ether:EtOAc=1:1) to give the title compound as colorless semisolid. The diastereomers were separated by Prep-Chiral-HPLC with the following conditions: Column: Chiralpak IA, 2*25 cm, 5 μm; Mobile Phase A: Hex (0.1% FA)-HPLC, Mobile Phase B: EtOH-HPLC; Flow rate: 20 mL/min; Gradient: 30 B to 30 B in 16 min; 220/254 nm; RT1: 8.535; RT2: 13.083; Injection Volumn: 1 mL; number of runs: 4; to give first eluting isomer (Example 6A, RT1: 8.535, 37.6 mg, 91.49% purity, 8.19% yield) as a white solid. LCMS (ESI) m/z calcd for C₄₀H₅₈FN₆O₉P: 816; found: 817 (M+1). ¹H NMR (400 MHz, DMSO) δ 8.23 (s, 1H), 7.92-7.76 (m, 2H), 7.26-7.11 (m, 5H), 6.26-6.22 (m, 1H), 5.75 (d, J=5.6 Hz, 1H), 5.63 (t, J=11.6 Hz, 1H), 4.54-4.41 (m, 2H), 4.03-3.82 (m, 7H), 3.65 (s, 1H), 2.94-2.78 (m, 2H), 2.73-2.63 (m, 1H), 2.45-2.37 (m, 1H), 1.56-1.35 (m, 4H), 1.28-1.07 (m, 23H), 0.86-0.78 (m, 6H); and the second eluting isomer (Example 6B, RT2: 13.083, 162.8 mg, 98.0% purity, 38.0% yield) as pale yellow semisolid. LCMS (ESI) m/z calcd for C₄₀H₅₈FN₆O₉P: 816; found: 817 (M+1). ¹H NMR (400 MHz, Chloroform-d) δ 8.23 (s, 1H), 8.05 (br, s, 2H), 7.28-7.16 (m, 5H), 6.36 (dd, J=3.2, 7.6 Hz, 1H), 4.74-4.65 (m, 2H), 4.36-4.31 (m, 1H), 4.26-4.06 (m, 6H), 3.79-3.70 (m, 2H), 3.14-3.06 (m, 1H), 3.03-2.94 (m, 1H), 2.80-2.61 (m, 3H), 1.67-1.52 (m, 4H), 1.38-1.19 (m, 23H), 0.90-0.83 (m, 6H).

### Example 7

### Nonyl ((((2R,3S,5R)-5-(6-amino-2-fluoro-9H-purin-9-yl)-2-ethynyl-3-hydroxytetrahydrofuran-2-yl)methoxy)(2-(nonyloxy)-2-oxoethoxy)phosphoryl)-L-phenylalaninate

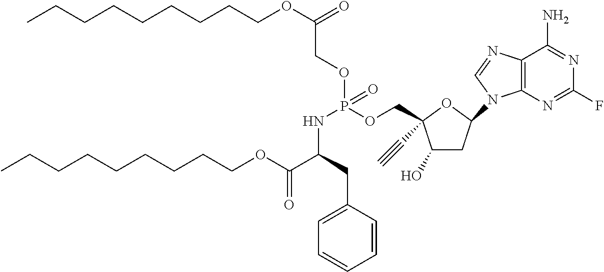 [SMILES: C#C[C@]1(COP(=O)(N[C@@H](Cc2ccccc2)C(=O)OCCCCCCCCC)OCC(=O)OCCCCCCCCC)O[C@@H](n2cnc3c(N)nc(F)nc32)C[C@@H]1O]

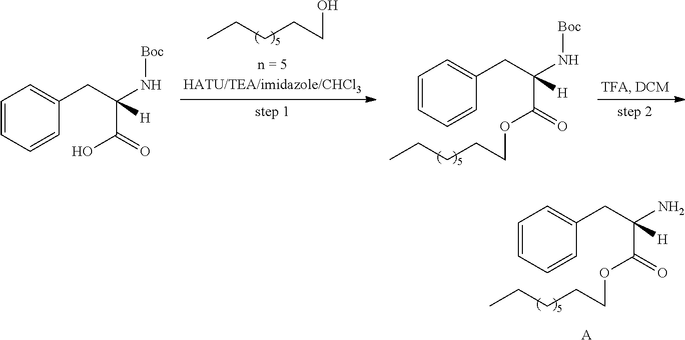

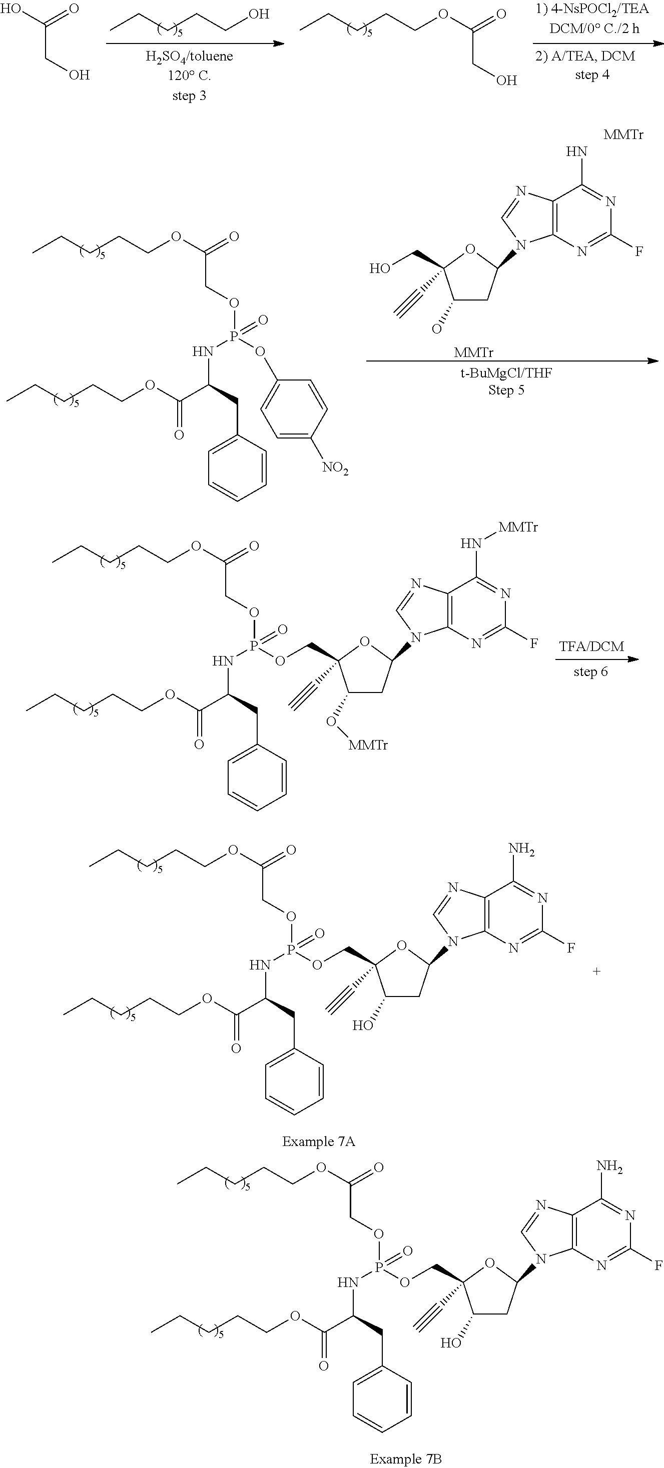

### Step 1: Nonyl (tert-butoxycarbonyl)-L-phenylalaninate

To a stirred mixture of (tert-butoxycarbonyl)-L-phenylalanine (15 g, 56.5 mmol) in chloroform (100 mL) was added HATU (32.2 g, 85 mmol), nonan-1-ol (12.23 g, 85 mmol), TEA (23.64 mL, 170 mmol) and imidazole (11.55 g, 170 mmol) at 0° C. The reaction mixture was stirred for 5 h at room temperature. LCMS indicated completion of. reaction. The reaction was diluted with water (100 mL), extracted with DCM (2×100 mL) and the combined organic extracts were washed with saturated aqueous NaHCO₃ (2×100 mL) and brine (50 mL). The mixture was concentrated to dryness under vacuum. The crude product was purified by flash column chromatography (silica gel, 330 g, pet. ether:EtOAc=10:1) to give the product (13 g, 93%, yield: 54.6%) as yellow oil. LCMS (ESI) m/z calcd for C₂₃H₃₇NO₄: 391; found: 392 (M+1).

### Step 2: Nonyl L-phenylalaninate

To a stirred mixture of nonyl (tert-butoxycarbonyl)-L-phenylalaninate (13.0 g, 33.2 mmol) in DCM (80 mL) was added TFA (15.0 mL, 195 mmol) at 0° C. The reaction mixture was stirred for 1 h at room temperature. LCMS indicated completion of. reaction. The reaction mixture was quenched with saturated aqueous NaHCO₃ (30 mL). The organic layer separated and water layer was extracted with DCM (20 mL*3). The combined organic layers dried, and concentrated to dryness under vacuum to afford nonyl L-phenylalaninate (8 g, 23.33 mmol, 70.3% yield) as yellow oil. LCMS (ESI) m/z calcd for C18H₂₉NO₂: 291; found: 292 (M+1). ¹H NMR (400 MHz, Chloroform-d) δ 7.44-7.13 (m, 5H), 4.22-3.97 (m, 2H), 3.82-3.52 (m, 1H), 3.15-2.68 (m, 2H), 1.63 (d, J=3.7 Hz, 2H), 1.62-1.58 (m, 2H), 1.34-1.26 (m, 12H), 0.95-0.79 (m, 3H).

### Step 3: Nonyl 2-hydroxyacetate

A solution of 2-hydroxyacetic acid (5 g, 65.7 mmol), nonan-1-ol (18.97 g, 131 mmol) and H2SO4 (0.50 ml, 9.38 mmol) in toluene (50 mL) was heated at refulx for 4 hours and the water generated was removed with Dean-Stark. The reaction mixture was concentrated to dryness in vaccum. The residue was diluted with water (100 mL) and extracted with EtOAc (100 mL). The organic layer was washed with saturated aqueous NaHCO₃, dried over anhydrous sodium sulfate and concentrated to dryness under vaccum. The crude product was purified by flash column chromatography (silica gel, 330 g, pet. ether:EtOAc=2:1) to give the title compound (14 g, 50%, yield: 52.6%) as pale yellow oil. LCMS (ESI) m/z calcd for C₁₁H₂₂O₃: 202; found: 203 (M+1). ¹H NMR (400 MHz, Chloroform-d) δ 4.19 (t, J=6.8 Hz, 2H), 4.14 (s, 2H), 3.63 (t, J=6.4 Hz, 2H), 1.69-1.60 (m, 2H), 1.56 (d, J=8.0 Hz, 2H), 1.40-1.17 (m, 24H), 0.92-0.80 (m, 6H).

### Step 4: nonyl ((4-nitrophenoxy)(2-(nonyloxy)-2-oxoethoxy)phosphoryl)-L-phenylalaninate

To a solution of nonyl 2-hydroxyacetate_(3.5 g, 17.30 mmol) and 4-nitrophenyl phosphorodichloridate (4.43 g, 17.30 mmol) in DCM (35 mL) stirred under nitrogen at 0° C. was added TEA (2.41 mL, 17.3 mmol). The resulting mixture was stirred at 0° C. for 2 hours. Then nonyl L-phenylalaninate (4 g, 13.73 mmol) in DCM (40 mL) and TEA (4.82 mL, 34.6 mmol) were added dropwise. The reaction mixture was stirred at 0° C. for 1 h and then stirred at 15° C. for 16 h. LCMS indicated completion of. reaction. The resulting mixture was filtered and the filtrate was concentrated to dryness in vaccum. The crude product was purified by flash column chromatography (silica gel, 330 g, pet. ether:EtOAc=2:1) to give the title compound (4.4 g) as an oil. The crude product was subjected to reverse phase-HPLC purification (C18, 0-100% MeCN/water with 0.1% FA) to give the title compound (3.2 g, 96%, yield: 26%) as white solid. LCMS (ESI) m/z calcd for C₃₅H₅₃N₂O₉P: 676; found: 677 (M+1). ¹H NMR (400 MHz, Chloroform-d) δ 8.22-8.11 (m, 2H), 7.34-7.27 (m, 2H), 7.26-7.22 (m, 3H), 7.15-7.11 (m, 2H), 4.53-4.25 (m, 3H), 4.23-3.98 (m, 4H), 3.68-3.66 (m, 1H), 3.14-3.06 (m, 1H), 2.99-0.96 (m, 1H), 1.62 (d, J=6.8 Hz, 4H), 1.40-1.19 (m, 24H), 0.88 (t, J=6.8 Hz, 6H).

### Step 5: Nonyl ((((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methoxy)(2-(nonyloxy)-2-oxoethoxy)phosphoryl)-L-phenylalaninate

To a solution of. ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (1 g, 1.193 mmol) in tetrahydrofuran (5 mL) stirred under nitrogen at 15° C. was added a solution of 1M tert-butylmagnesium chloride/THF (1.790 mL, 1.790 mmol) dropwise. The reaction mixture was stirred at 15° C. for 2 hours. Then, nonyl ((4-nitrophenoxy)(2-(nonyloxy)-2-oxoethoxy)phosphoryl)-L-phenylalaninate (1.615 mg, 2.387 mmol) in tetrahydrofuran (3 mL) was added dropwise at 15° C. The reaction was stirred at 30° C. for 16 hours. LCMS indicated completion of. reaction. The crude product was purified by flash column chromatography (silica gel, 40 g, pet. ether:EtOAc=1:2) to give the product which was further purified by reverse phase-HPLC purification ((C18, 0-100% MeCN/water with 0.1% FA) to give the title compound (700 mg, 90%, yield: 38%) as white solid. LCMS (ESI) m/z calcd for C₈₁H₉₂FN₆O₁₁P: 1375; found: 1376 (M+1). ¹H NMR (400 MHz, Chloroform-d) δ 7.94-7.90 (m, 1H), 7.55-7.51 (m, 4H), 7.38 (d, J=8.8 Hz, 2H), 7.34-7.26 (m, 7H), 7.26-7.13 (m, 13H), 7.13-7.05 (m, 2H), 6.83-6.75 (m, 4H), 6.24-6.12 (m, 1H), 4.62-4.44 (m, 1H), 4.25 (d, J=11.2 Hz, 1H), 4.19 (d, J=11.2 Hz, 1H), 4.13-4.11 (m, 5H), 3.97 (t, J=6.8 Hz, 1H), 3.77 (s, 3H), 3.74 (s, 3H), 3.45-3.24 (m, 1H), 2.92 (d, J=6.0 Hz, 2H), 2.86-2.81 (m, 1H), 1.89 (s, 1H), 1.81-1.67 (m, 1H), 1.68-1.44 (m, 5H), 1.28-1.20 (m, 24H), 0.88 (td, J=6.8, 1.6 Hz, 6H).

### Step 6: Nonyl ((((2R,3S,5R)-5-(6-amino-2-fluoro-9H-purin-9-yl)-2-ethynyl-3-hydroxytetrahydrofuran-2-yl)methoxy)(2-(nonyloxy)-2-oxoethoxy)phosphoryl)-L-phenylalaninate

To a solution of nonyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(2-(nonyloxy)-2-oxoethoxy)phosphoryl)-L-phenylalaninate (650 mg, 0.473 mmol) in DCM (13 mL) stirred in air at 15° C. was added TFA (1.3 mL) dropwise. The reaction mixture was stirred at 15° C. for 2 hours. LCMS traces showed the reaction was completed. The reaction was quenched with saturated aqueous NaHCO₃ and pH was adjusted to 8-9, and the organic layer was separated. The water phase was extracted with DCM (10 mL), the combined organic layers were dried over anhydrous sodium sulfate and concentrated to dryness under vaccum. The residue was subjected to reverse phase-HPLC purification (C18, 10-100% MeCN/water with 0.1% NH₄HCO₃) to give the title compound (400 mg) as as a white semi-solid. The diastereomers were separated by chiral-HPLC (Column: CHIRALPAK IF, 2*25 cm, 5 μm; Mobile Phase A: Hex:DCM=3:1 (0.1% FA)-HPLC, Mobile Phase B: IPA-HPLC; Flow rate: 16 mL/min; Gradient: 30 B to 30 B in 10 min; 220/254 nm; RT1: 5.159; RT2: 8.215; Injection Volumn: 4 ml; number of runs: 5 to give first eluting isomer (Example 7A 130 mg) and second eluting isomer (Example 7B 140 mg). The first eluting isomer was (130 mg) was repurified with Achiral-SFC (Column: DAICEL DCpak P4VP, 20 mm*250 mm, 5 μm; Mobile Phase A:CO₂, Mobile Phase B: MeOH (8 mmol/L NH3 MeOH)-HPLC; Flow rate: 50 mL/min; Gradient: 25% B; 254 nm; RT: 4.63; Injection Volumn: 0.8 ml; number of runs: 11; to give Example 7A (86.9 mg, 99.15%, yield: 21.94%) as yellow solid. LCMS (ESI) m/z calcd for C₄₁H₆₀FN₆O₉P: 830. Found: 831 (M+1). ¹H NMR (400 MHz, Chloroform-d) δ 8.14 (s, 1H), 7.30-7.23 (m, 2H), 7.18-7.16 (m, 3H), 6.36-6.33 (m, 1H), 6.10 (br s, 1H), 4.90-4.85 (m, 1H), 4.40-4.36 (m, 1H), 4.26-4.24 (m, 1H), 4.17-4.08 (m, 7H), 3.52-3.48 (m, 1H), 3.20-3.10 (m, 1H), 2.98-2.90 (m, 1H), 2.78-2.72 (m, 3H), 1.64-1.57 (m, 4H), 1.1.35-1.20 (m, 24H), 0.90-0.86 (m, 6H). The second elutin isomer (140 mg) was repurified with Achiral-SFC (Column: DAICEL DCpak P4VP, 20 mm*250 mm, 5 μm; Mobile Phase A: CO₂, Mobile Phase B: IPA (8 mmol/L NH₃ MeOH)-HPLC; Flow rate: 50 mL/min; Gradient: 28% B; 254 nm; RT: 4.82; Injection Volumn: 0.5 ml; number of runs: 13 to give Example 7B (78 mg, 98.07%, yield: 19.48%) as yellow solid. LCMS (ESI) m/z calcd for C₄₁H₆₀FN₆O₉P: 830. Found: 831 (M+1). ¹H NMR (400 MHz, Chloroform-d) δ 8.13 (s, 1H), 7.26-7.25 (m, 1H), 7.23-7.17 (m, 4H), 6.38-6.35 (m, 1H), 6.10 (br s, 1H), 4.75-4.68 (m, 1H), 4.48-4.37 (m, 1H), 4.22-4.08 (m, 8H), 3.71-3.63 (m, 1H), 3.17-3.08 (m, 1H), 2.88-2.80 (m, 1H), 2.73-2.67 (m, 3H), 1.63-1.58 (m, 4H), 1.30-1.25 (m, 24H), 0.89-0.86 (m, 6H).

### Example 8

### Nonyl ((((2R,3S,5R)-5-(6-amino-2-fluoro-9H-purin-9-yl)-2-ethynyl-3-hydroxytetrahydrofuran-2-yl)methoxy)(((S)-1-(nonyloxy)-1-oxopropan-2-yl)oxy)phosphoryl)-L-phenylalaninate

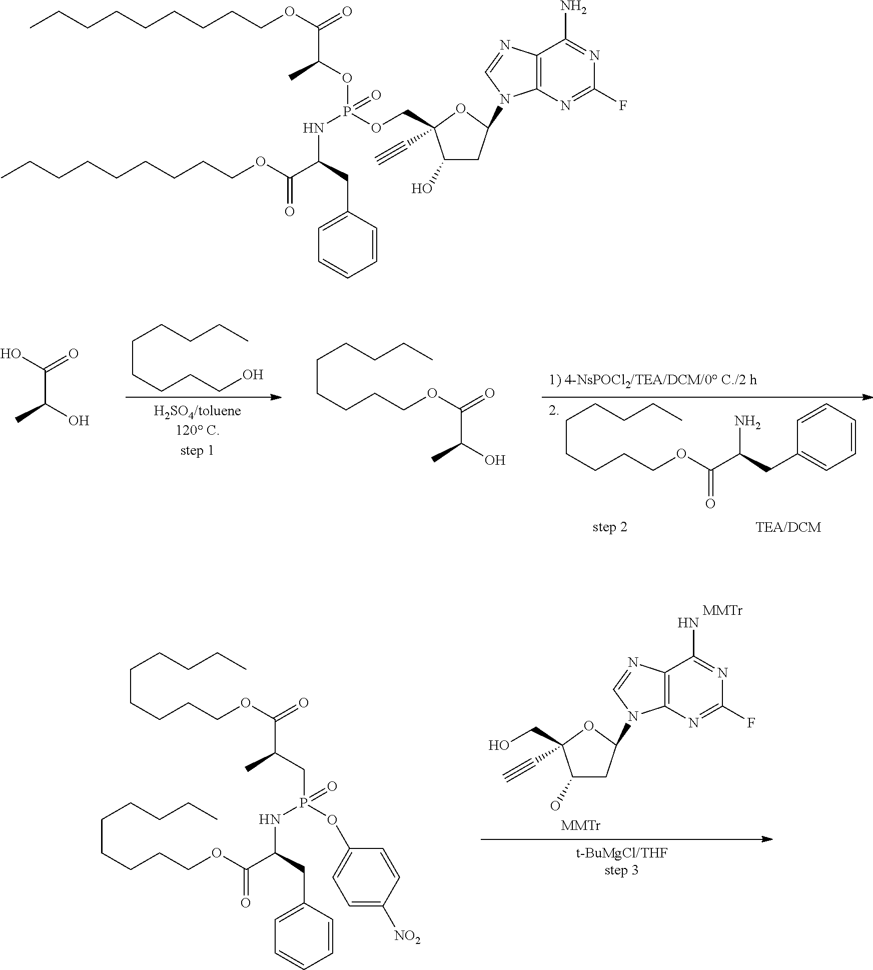

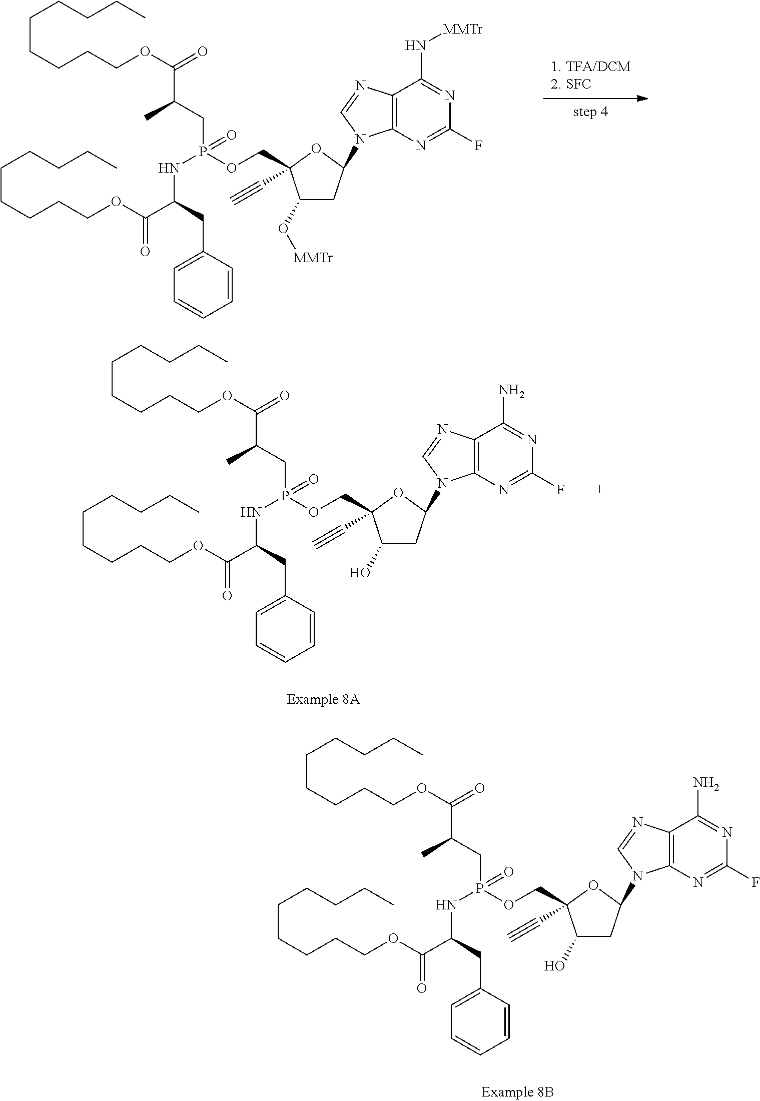

### Step 1: Nonyl (S)-2-hydroxypropanoate

A mixture of nonan-1-ol (16.01 g, 111 mmol), (S)-2-hydroxypropanoic acid (5 g, 55.5 mmol) and sulfuric acid (1 ml, 55.5 mmol) in toluene (50 mL) was heated at 120° C. for 16 hours and the water generated was removed with Dean-Stark condenser. The reaction was evaporated to dryness and the residue was diluted with EtOAc (100 mL). The resulting solution was washed with water (30 mL), aqueous NaHCO₃ (30 mL), brine (30 mL), dried over anhydrous sodium sulfate and concentrated to dryness under vaccum. The crude product was purified by flash column chromatography (silica gel, 330 g, pet. ether:EtOAc=2:1) to give the title compound (9 g, 80%, yield: 60%) as colorless oil. LCMS (ESI) m/z calcd for C₁₂H₂₄O₃: 216; found: 217 (M+1). ¹H NMR (300 MHz, Chloroform-d) δ 4.26 (q, J=6.9 Hz, 1H), 4.23-4.11 (m, 2H), 1.67-1.64 (m, 2H), 1.41 (d, J=6.9 Hz, 3H), 1.34-1.24 (m, 12H), 0.89-0.86 (m, 3H).

### Step 2: Nonyl ((4-nitrophenoxy)(((S)-1-(nonyloxy)-1-oxopropan-2-yl)oxy)phosphoryl)-L-phenylalaninate

To a solution of nonyl (S)-2-hydroxypropanoate (2 g, 9.25 mmol) and 4-nitrophenyl phosphorodichloridate (2.36 g, 9.25 mmol) in DCM (20 mL) stirred under nitrogen at 0° C. was added TEA (1.29 mL, 9.25 mmol). The resulting mixture was stirred at 0° C. for 2 hours. Then nonyl L-phenylalaninate (2.69 g, 9.25 mmol) in DCM (20 mL) and TEA (2.58 mL, 18.5 mmol) were added dropwise. The reaction mixture was stirred at 0° C. for 1 h and then stirred at 15° C. for 16 h. LCMS indicated completion of. reaction. The resulting mixture was washed with water (50 mL), brine (50 mL), dried over anhydrous sodium sulfate and concentrated to dryness in vaccum. The residue was subjected to reverse phase-HPLC purification (C18, 10-100% MeCN/water with 0.1% TFA) to give the title compound (4 g, 90%, yield: 56%) as white solid. LCMS (ESI) m/z calcd for C₃₆H₅₅N₂O₉P: 690; found: 691 (M+1). ¹H NMR (400 MHz, Chloroform-d) δ 8.20-8.16 (m, 2H), 7.34-7.28 (m, 2H), 7.27 (s, 1H), 7.25-7.20 (m, 2H), 7.13 (t, J=7.2 Hz, 2H), 4.81-4.77 (m, 1H), 4.44-4.26 (m, 1H), 4.07-4.05 (m, 3H), 3.65-3.61 (m, 1H), 3.06-3.04 (m, 2H), 1.64 (d, J=6.4 Hz, 2H), 1.58-1.55 (m, 2H), 1.48 (d, J=6.8, 3H), 1.31-1.21 (m, 24H), 0.91-0.85 (m, 6H).

### Step 3: Nonyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methoxy)(((S)-1-(nonyloxy)-1-oxopropan-2-yl)oxy)phosphoryl)-L-phenylalaninate

To a solution of. ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (1 g, 1.193 mmol) in THF (10 mL) stirred under nitrogen at 15° C. was added a solution of tert-butylmagnesium chloride/THF (1.790 mL, 1.790 mmol) in THF (3 mL) dropwise. The reaction mixture was stirred at 15° C. for 2 hours. Then, nonyl ((4-nitrophenoxy)(((S)-1-(nonyloxy)-1-oxopropan-2-yl)oxy)phosphoryl)-L-phenylalaninate (104 mg, 0.15 mmol) in THF (3 mL) was added. The reaction mixture was stirred at 15° C. for 16 hours. LCMS indicated completion of. reaction. The reaction mixture was concentrated and the residue was subjected to reverse phase-HPLC purification (C18, 10-100% MeCN/water with 0.1% NH₄CO₃) to give the title compound (850 mg, 90%, yield: 46%) as white solid. LCMS (ESI) m/z calcd for C₈₂H₉₄FN₆O₁₁P: 1389; found: 1390 (M+1).

### Step 4: Nonyl ((((2R,3S,5R)-5-(6-amino-2-fluoro-9H-purin-9-yl)-2-ethynyl-3-hydroxytetrahydrofuran-2-yl)methoxy)(((S)-1-(nonyloxy)-1-oxopropan-2-yl)oxy)phosphoryl)-L-phenylalaninate

To a solution of. nonyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran yl)meth-oxy)(((S)-1-(nonyloxy)-1-oxopropan-2-yl)oxy)phosphoryl)-L-phenylalaninate (850 mg, 0.612 mmol) in DCM (10 mL) stirred in air at 15° C. was added TFA (1 mL) and stirred at 15° C. for 2 hours. LCMS indicated completion of. reaction. The reaction was quenched with saturated aqueous NaHCO₃ and pH was adjusted to 8. The organic layer was separated and aqueous layer was extracted with DCM (10 mL). The combined organic layers were dried over anhydrous sodium sulfate and concentrated to dryness under vaccum. The crude product was subjected to reverse phase-HPLC purification (10-100% MeCN/water with 0.1% NH₄CO₃) to give product (185 mg) as white solid. The isomers were separated by Prep-Chiral-HPLC (Column: CHIRAL ART Cellulose-SB, 2*25 cm, 5 μm; Mobile Phase A: Hex (0.1% TFA)-HPLC, Mobile Phase B: EtOH-HPLC; Flow rate: 20 mL/min; Gradient: 15 B to 15 B in 14 min; 220/254 nm; RT1: 9.074; RT2: 11.395; Injection Volumn: 0.5 ml; number of runs: 17 to give first eluating isomer (Example 8A, 52 mg, 87%) as a yellow solid (This was repurified with Achiral-SFC (Column: DAICEL DCpak P4VP, 20 mm*250 mm, 5 μm; Mobile Phase A:CO₂, Mobile Phase B: MeOH:DCM=1:1 (2 M NH3-MEOH); Flow rate: 50 mL/min; Gradient: 20% B; 254 nm; RT1: 3.23; Injection Volumn: 0.8 ml; number of runs: 5 to give first eluting isomer Example 8A (19.9 mg, 98.14%, 3.78%) as yellow solid) LCMS (ESI) m/z calcd for C₄₂H₆₂FN₆O₉P: 844; found: 845 (M+1). ¹H NMR (400 MHz, Chloroform-d) δ 8.26 (s, 1H), 7.29-7.26 (m, 1H), 7.24-7.22 (m, 2H), 7.11-7.09 (m, 2H), 6.92-6.88 (m, 1H), 6.39-6.37 (m, 1H), 4.97-4.84 (m, 2H), 4.74-4.72 (m, 1H), 4.53 (br s, 2H), 4.35-4.33 (m, 2H), 4.09-4.05 (m, 4H), 3.12-3.11 (m, 2H), 2.79-2.76 (m, 2H), 2.70 (s, 1H), 1.68-1.59 (m, 4H), 1.51-1.49 (d, J=6.4 Hz, 3H), 1.27-1.25 (m, 24H), 0.90-0.86 (m, 6H); and second eluting isomer (Example 8B, 47.3 mg, 95.11%, yield: 8.7%) as a yellow solid. LCMS (ESI) m/z calcd for C₄₂H₆₂FN₆O₉P: 844; found: 845 (M+1). ¹H NMR (400 MHz, Chloroform-d) δ 8.22 (s, 1H), 7.31-7.29 (m, 1H), 7.26-7.24 (m, 2H), 7.12-7.11 (m, 2H), 6.96-6.94 (m, 1H), 6.39-6.36 (m, 1H), 5.38 (br s, 2H), 4.85-4.84 (m, 1H), 4.72-4.67 (m, 2H), 4.38-4.36 (m, 1H), 4.28-4.26 (m, 1H), 4.08-4.03 (m, 4H), 3.20-3.18 (m, 1H), 3.11-3.09 (m, 1H), 2.80-2.74 (m, 2H), 2.72 (s, 1H), 1.62-1.59 (m, 4H), 1.47-1.45 (d, J=6.4 Hz, 3H) 1.26-1.24 (m, 24H), 0.90-0.85 (m, 6H).

### Example 9

### Decyl ((((2R,3S,5R)-5-(6-amino-2-fluoro-9H-purin-9-yl)-2-ethynyl-3-hydroxytetrahydrofuran-2-yl)methoxy)(2-(decyloxy)-2-oxoethoxy)phosphoryl)-L-phenylalaninate

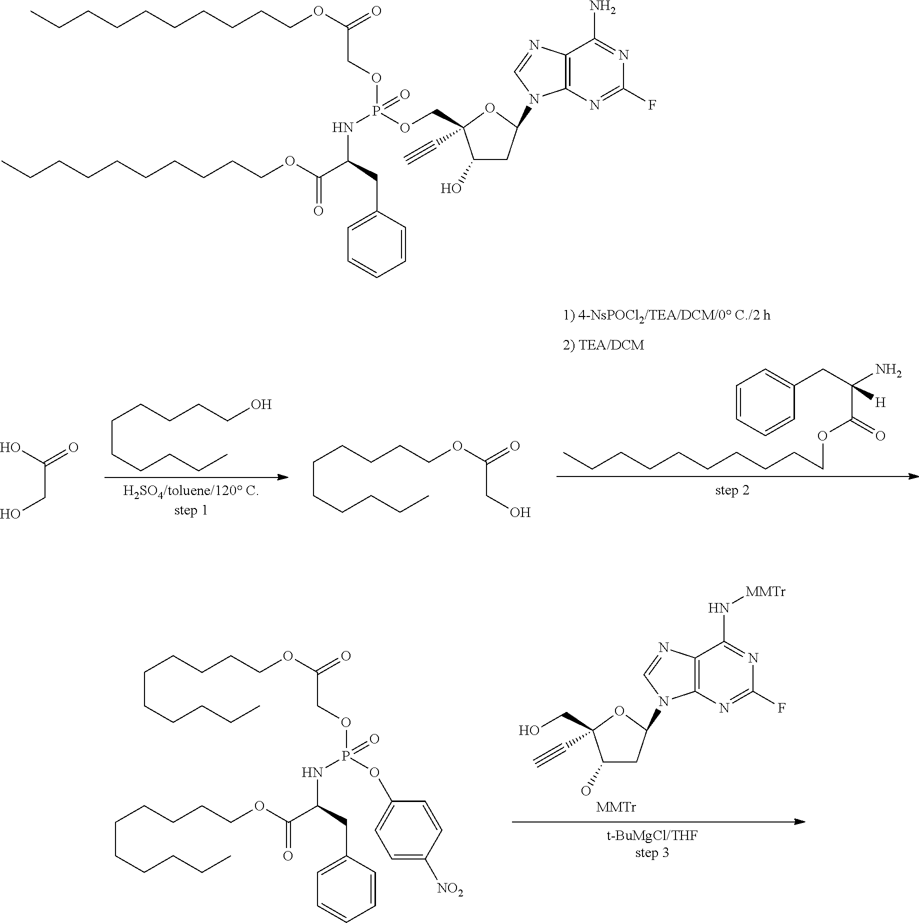

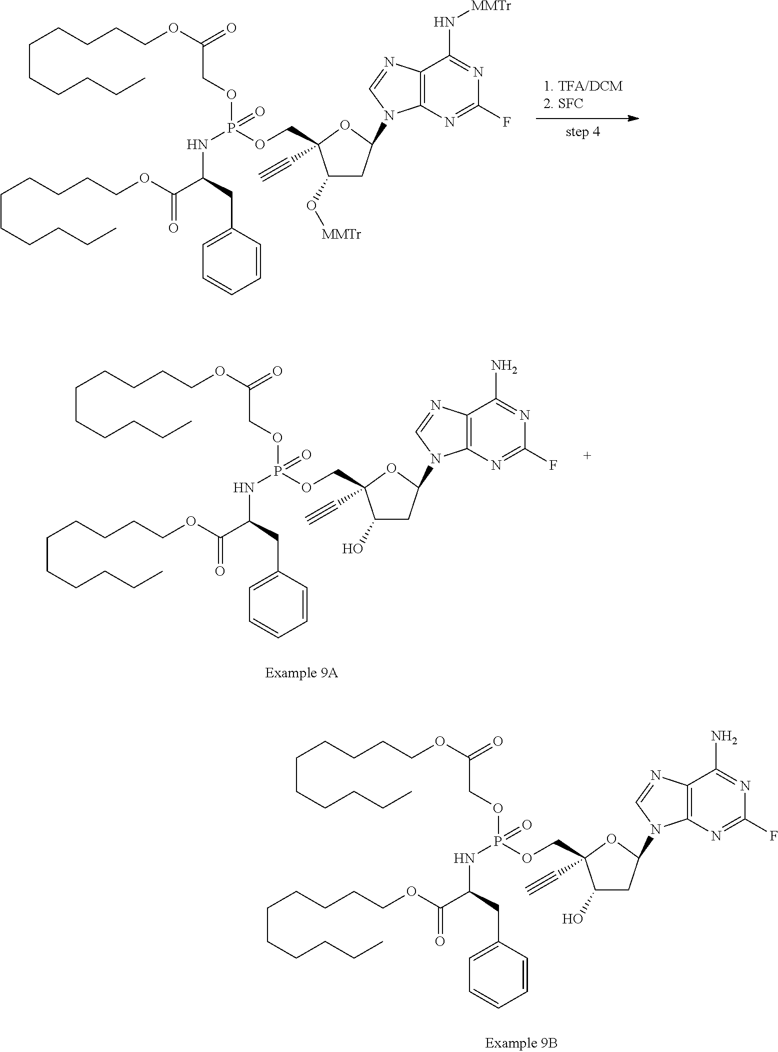

### Step 1: decyl 2-hydroxyacetate

To a mixture of. 2-hydroxyacetic acid (5 g, 65.7 mmol), decan-1-ol (20.81 g, 131 mmol) and sulfuric acid (0.5 mL, 9.38 mmol) in toluene (50 mL) was stirred at 120° C. overnight. TLC showed the reaction was completed (stained with KMnO₄, pet. ether:EtOAc=1:1). The reaction mixture was concentrated and residue was partitioned between EtOAC (50 mL) and water (50 mL). The organic phase was washed with NaHCO₃ (aq) 50 mL, dried. over sodium sulphate and concentrated to give the crude product as white solid. The crude product was added to silica gel column (330 g) and was eluted with 0-10% EtOAc/Hex. The fractions containing desired product was evaporated to give decyl 2-hydroxyacetate (14 g, 38.8 mmol, 59.1% yield) as white solid. LCMS (ESI) m/z calcd for C₁₂H₂₄O₃:216; found: 217 (M+1).

### Step 2: Decyl ((2-(decyloxy)-2-oxoethoxy)(4-nitrophenoxy)phosphoryl)-L-phenylalaninate

To a mixture of 4-nitrophenyl phosphorodichloridate (4.73 g, 18.49 mmol) and decyl 2-hydroxyacetate (4 g, 18.49 mmol) in DCM (120 mL) was added TEA (2.83 mL, 20.3 mmol) dropwise at 0° C. The reaction was stirred for 2 h at 0° C. Then decyl L-phenylalaninate (5.65 g, 18.49 mmol) in DCM (40 mL) and TEA (5.68 mL, 40.7 mmol) was added at 0° C. The reaction mixture was stirred for 1 h at 0° C. LCMS showed the reaction was completed. The reaction mixture was quenched with water (10 mL), extracted with DCM (3*10 mL). The combined organic layers were washed with brine and concentrated to dryness. The residue was purified by reverse phase column (330 g, ACN/water (0.05% NH4HCO3) to give decyl ((2-(decyloxy)-2-oxoethoxy)(4-nitrophenoxy)phosphoryl)-L-phenylalaninate (2.1 g, 2.83 mmol, 15.31% yield) as yellow oil. LCMS (ESI) m/z calcd for C₃₇H₅₇N₂O₉P: 704; found: 705 (M+1).

### Step 3: Decyl ((2-(decyloxy)-2-oxoethoxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate

To a solution of ((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methanol (1.2 g, 1.432 mmol). in THF (12 mL) stirred under nitrogen at room temperature was added a solution of tert-butylmagnesium chloride/THF (2.148 mL, 2.148 mmol). The reaction mixture was stirred at 25° C. for 1 hour. Then, the decyl ((2-(decyloxy)-2-oxoethoxy)(4-nitrophenoxy)phosphoryl)-L-phenylalaninate (2.019 g, 2.86 mmol) was added at 25° C. The resulting mixture was stirred at room temperature for 6 hour. LCMS showed completion of reaction. The reaction mixture was concentrated to dryness, and the residue was purified by silica gel column (120 g, pet. ether:EtOAc=1:1) to afford decyl ((2-(decyloxy)-2-oxoethoxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenyl-methoxy)tetrahydrofuran-2-yl)methoxy)phosphoryl)-L-phenylalaninate (1.0 g, 0.712 mmol, 49.7% yield). as yellow solid. LCMS (ESI) m/z calcd for C₈₃H₉₆FN₆O₁₁P: 1403; found: 1404 (M+1).

### Step 4: Decyl ((((2R,3S,5R)-5-(6-amino-2-fluoro-9H-purin-9-yl)-2-ethynyl-3-hydroxytetrahydrofuran-2-yl)methoxy)(2-(decyloxy)-2-oxoethoxy)phosphoryl)-L-phenylalaninate

To a solution of decyl ((2-(decyloxy)-2-oxoethoxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate (900 mg, 0.641 mmol) in DCM (9 mL) was added TFA (0.9 mL) at room temperature and stirred for 3 h. LCMS showed completion of reaction. The pH was adjusted to 6-7 with saturated aqueous NaHCO₃ and extracted with DCM (3*10 mL). The combined organic layers were washed with brine and dried over Na2SO4. After filtration, the filtrate was concentrated and the residue was purified on C18 column (Mobile Phase A: Water (0.05% TFA), Mobile Phase B: ACN; Flow rate: 80 mL/min; (CAN=100%) to afford decyl ((((2R,3S,5R)-5-(6-amino-2-fluoro-9H-purin-9-yl)-2-ethynyl-3-hydroxytetrahydrofuran-2-yl)methoxy)(2-(decyloxy)-2-oxoethoxy)phosphoryl)-L-phenylalaninate (450 mg) as yellow oil. The isomers were separated by following condition: Column: CHIRALPAK IH, 2.0*25 cm, 5 μm; Mobile Phase A: CO2, Mobile Phase B: MEOH (2 mM NH3-MEOH); Flow rate: 40 mL/min; Gradient: 35% B; 220 nm; RT1: 3.39; RT2: 4.57; Injection Volumn: 2 ml; number of runs: 20; the fractions containing desired compounds were concentrated to dryness under vacuum to provide two isomers. The first eluting isome (Example 9A, RT1: 3.39) was recrystallized from MeOH/H2O in the ratio of 1:1. The solid was collected by filtration and dried under sun lamp (45° C.) to afford 43 mg (0.049 mmol, 7.69% yield) as yellow amorphous solid. LCMS (ESI) m/z calcd for C₄₃H₆₄FN₆O₉P: 858; found: 859 (m+1). ¹H NMR (400 MHz, CDCl₃). δ 8.21 (s, 1H), 7.28-7.17 (m, 5H), 6.37-6.34 (m, 1H), 4.68 (d, J=4 Hz, 1H), 4.43-4.31 (m, 1H), 4.22-4.08 (m, 7H), 3.72-3.63 (m, 2H), 3.11 (t, J=8 Hz, 1H), 2.96-2.91 (m, 1H), 2.74-2.70 (m, 2H), 2.05 (s, 1H), 1.65-1.57 (m, 4H), 1.34-1.25 (m, 30H), 0.88 (t, J=8 HZ 6H). The second eluting isome (Example 9B, RT2: 4.57) was re-crystallized from MeOH. The solid was collected by filtration and dried under sun lamp (45° C.) to afford 144 mg (0.163 mmol, 25.5% yield) as white amorphous solid. LCMS (ESI) m/z calcd for C₄₃H₆₄FN₆O₉P: 858; found: 859 (m+1). ¹H NMR (400 MHz, CDCl₃). δ 8.30 (s, 1H), 7.31-7.17 (m, 5H), 6.37-6.34 (m, 1H), 4.84 (s, 1H), 4.41-4.05 (m, 10H), 3.51 (t, J=12 HZ 1H), 3.19-3.15 (m, 1H), 2.98-2.92 (m, 1H), 2.78-2.72 (m, 3H), 1.64-1.58 (m, 4H), 1.34-1.24 (m, 30H), 0.90-0.58 (m, 6H).

### Example 10

### Decyl ((((2R,3S,5R)-5-(6-amino-2-fluoro-9H-purin-9-yl)-2-ethynyl-3-hydroxytetrahydrofuran-2-yl)methoxy)(((S)-1-(decyloxy)-1-oxopropan-2-yl)oxy)phosphoryl)-L-phenylalaninate

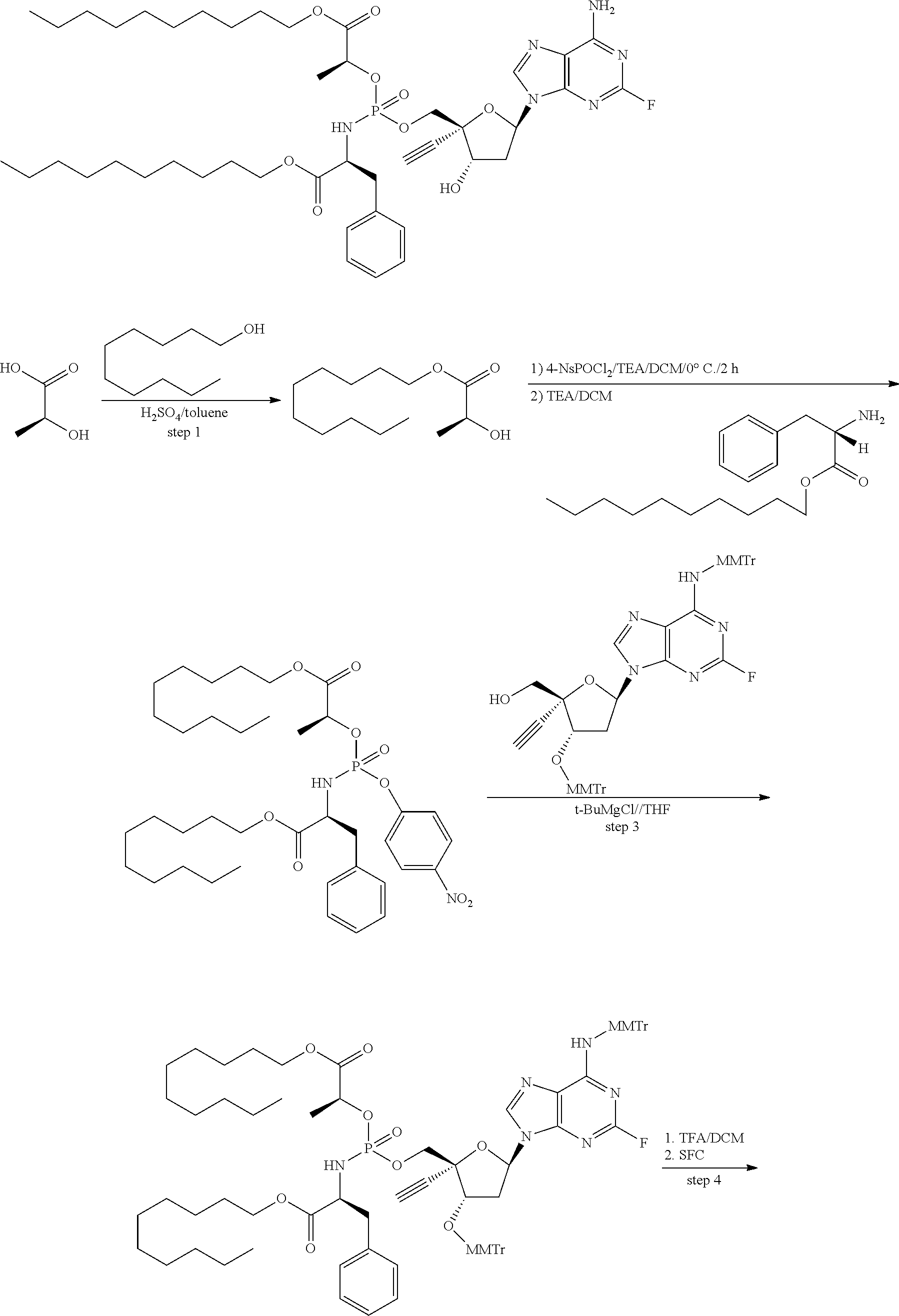

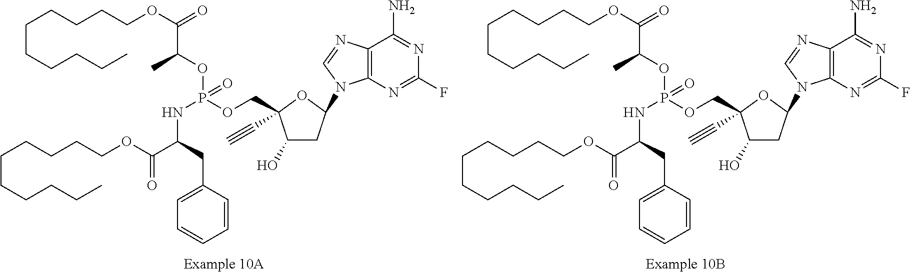 [SMILES: C#C[C@]1(COP(=O)(N[C@@H](Cc2ccccc2)C(=O)OCCCCCCCCCC)O[C@@H](C)C(=O)OCCCCCCCCCC)O[C@@H](n2cnc3c(N)nc(F)nc32)C[C@@H]1O ; C#C[C@]1(COP(=O)(N[C@@H](Cc2ccccc2)C(=O)OCCCCCCCCCC)O[C@@H](C)C(=O)OCCCCCCCCCC)O[C@@H](n2cnc3c(N)nc(F)nc32)C[C@@H]1O]

### Step 1: decyl (S)-2-hydroxypropanoate

To a stirred mixture of (S)-2-hydroxypropanoic acid (5.0 g, 55.5 mmol) in toluene (50 mL). was added decan-1-ol (17.57 g, 111 mmol) and H₂SO₄ (0.5 mL, 9.38 mmol) at room temperature. The reaction mixture was stirred for 6 h at room temperature. The reaction mixture was quenched with water (50 mL) and extracted with DCM (50 mL*3). The combined organic layers were washed with water (50 mL), brine (50 mL), dried over Na2SO4 and concentrated to dryness under vacuum to give the title compound which was used without purification. ¹H NMR (400 MHz, CDCl₃). δ 7.25-7.12 (m, 1H), 4.28-4.10 (m, 2H), 3.63-3.60 (m, 1H), 1.75-1.64 (m, 2H), 1.50-1.49 (m, 3H), 1.41-1.25 (m, 14H), 0.87 (t, J=4 Hz, 3H).

### Step 2: Decyl ((((S)-1-(decyloxy)-1-oxopropan-2-yl)oxy)(4-nitrophenoxy)phosphoryl)-L-phenylalaninate

To a stirred mixture of. 4-nitrophenyl phosphorodichloridate (5.0 g, 19.53 mmol) in DCM (50 mL) at room temp was added. decyl (S)-2-hydroxypropanoate (4.50 g, 19.53 mmol) and TEA (2 g) at room temperature. The reaction mixture was stirred at room temperature for 2 h. To this mixture was added a solution of decyl L-phenylalaninate (5 g, 16.37 mmol) in DCM (10 ml) and TEA (16.34 mL, 117 mmol). The reaction mixture was stirred at room temperature for 16 h. LCMS showed presence of desired product. The reaction mixture was quenched with water (50 mL), extracted with DCM (3*50 mL). The combined organic layers were washed with brine, dried over Na2SO4 and concentrated. The residue was purified by silica gel column (330 g, pet. ether:EtOAc=1:2) to afford decyl ((((S)-1-(decyloxy)-1-oxopropan-2-yl)oxy)(4-nitrophenoxy)phosphoryl)-L-phenylalaninate (5.0 g, 6.96 mmol, 35.6% yield) as a yellow oil. LCMS (ESI) m/z calcd for C₃₈H₅₉N₂O₉P: 718; found: 719 (M+1). ¹H NMR (300 MHz, CDCl₃). δ8.31-8.16 (m, 2H), 7.41-7.24 (m, 5H), 7.17-7.11 (m, 2H), 4.94-4.68 (m, 1H), 4.43-4.29 (m, 1H), 4.22-3.98 (m, 4H), 3.83-3.42 (m, 1H), 3.17-2.94 (m, 2H), 1.60-1.40 (m, 7H), 1.26 (br s, 27H), 0.90 (t, J=6 Hz, 6H).

### Step 3: Decyl ((((S)-1-(decyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate

To a solution of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (1.0 g, 1.193 mmol). in THF (12 mL) stirred under nitrogen at room temperature was added a solution of tert-butylmagnesium chloride/THF (1.790 mL, 1.790 mmol) and stirred at 25° C. for 1 hour. Then the decyl ((((S)-1-(decyloxy)-1-oxopropan-2-yl)oxy)(4-nitrophenoxy)phosphoryl)-L-phenylalaninate (1.716 g, 2.387 mmol) was added to the reaction at 25° C. The resulting mixture was stirred at room temperature for 6 hour. LCMS showed completion of reaction. The reaction mixture was concentrated and the residue was purified by silica gel column (120 g, pet. ether:EtOAc=1:1) to afford decyl ((((S)-1-(decyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenyl-methoxy)tetrahydrofuran-2-yl)methoxy)phosphoryl)-L-phenylalaninate (650 mg, 0.458 mmol, 38.4% yield). as yellow solid. LCMS (ESI) m/z calcd for C₈₄H₉₈FN₆O₁₁P: 1417; found: 1418 (M+1).

### Step 4: Decyl ((((2R,3S,5R)-5-(6-amino-2-fluoro-9H-purin-9-yl)-2-ethynyl-3-hydroxytetrahydrofuran-2-yl)methoxy)(((S)-1-(decyloxy)-1-oxopropan-2-yl)oxy)phosphoryl)-L-phenylalaninate

To a stirred mixture of decyl ((((S)-1-(decyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate (650 mg, 0.298 mmol) in DCM (6 mL) was added TFA (0.600 mL) at 0° C. and stirred for 2 h at room temperature. LCMS showed completion of reaction. The pH was adjusted to 6-7 with saturated aqueous NaHCO₃, and extracted with DCM (3*10 mL). The combined organic layers were washed with brine, and dried over Na2SO4. After filtration, the filtrate was concentrated and the residue was purified by silica gel column (80 g, MeOH:DCM=1:19) to afford decyl ((((2R,3S,5R)-5-(6-amino-2-fluoro-9H-purin-9-yl)-2-ethynyl-3-hydroxytetrahydrofuran-2-yl)methoxy)(((S)-1-(decyloxy)-1-oxopropan-2-yl)oxy)phosphoryl)-L-phenylalaninate (250 mg) as yellow oil. The isomers were separated by following condition: Column: DAICEL Dcpak P4VP, 4.6*50 mm, 3 μm; Mobile Phase A:, Mobile Phase B: ACN:MeOH=80:20 (20 mM NH3); Flow rate: 2 mL/min; Gradient: 5% B; 220 nm; RT1: 2.68; RT2: 2.87; the fractions containing desired compound were concentrated to give two isomers. First eluting isomer (Example 10A, RT1: 2.68) was re-crystallized from ACN/H₂O in the ratio of 1:1. The solid was collected by filtration and dried under sun lamp (45° C.) to afford 60 mg (0.066 mmol, 22.07% yield) as white crystal solid. LCMS (ESI) m/z calcd for C₄₄H₆₆FN₆O₉P: 872; found: 873 (m+1). ¹H NMR (300 MHz CDCl₃). δ 8.12 (s, 1H), 7.26-7.16 (m, 5H), 6.36 (s, 1H), 6.28-5.82 (m, 1H), 4.74 (t, J=6 Hz, 2H), 4.35-4.06 (m, 7H), 3.85 (s, 1H), 3.58 (s, 1H), 3.07-2.98 (m, 2H), 2.72 (s, 3H), 1.64-1.56 (m, 4H), 1.38 (d, J=3 Hz, 4H), 1.25 (s, 27H), 0.88 (t, J=6 Hz, 6H); and second eluting isomer (Example 10B, RT2: 2.87) was re-crystallized from ACN/H₂O. The solid was collected by filtration and dried under sun lamp (45° C.) to afford 48 mg (0.051 mmol, 17.28% yield) as white amorphous solid. LCMS (ESI) m/z calcd for C₄₄H₆₆FN₆O₉P: 872; found: 873 (m+1). ¹H NMR (300 MHz CDCl₃). δ 8.09 (s, 1H), 7.26 (s, 3H), 7.23-7.14 (m, 2H), 6.35 (t, J=6 Hz, 1H), 6.09 (s, 1H), 4.80 (s, 1H), 4.56 (t, J=9 Hz, 1H), 4.31-4.08 (m, 7H), 3.81 (s, 1H), 3.55-3.47 (m, 1H), 3.16 (t, J=3 Hz, 1H), 3.00-2.93 (m, 1H), 1.74 (t, J=3 Hz, 3H), 2.03 (s, 1H), 1.73-1.55 (m, 5H), 1.49 (d, J=3 Hz, 3H), 1.26 (br s, 27H), 0.88 (t, J=6 Hz, 6H).

### Example 11

### Dodecyl ((((2R,3S,5R)-5-(6-amino-2-fluoro-9H-purin-9-yl)-2-ethynyl hydroxytetrahydrofuran-2-yl)methoxy)(2-(dodecyloxy)-2-oxoethoxy)phosphoryl)-L-phenylalaninate

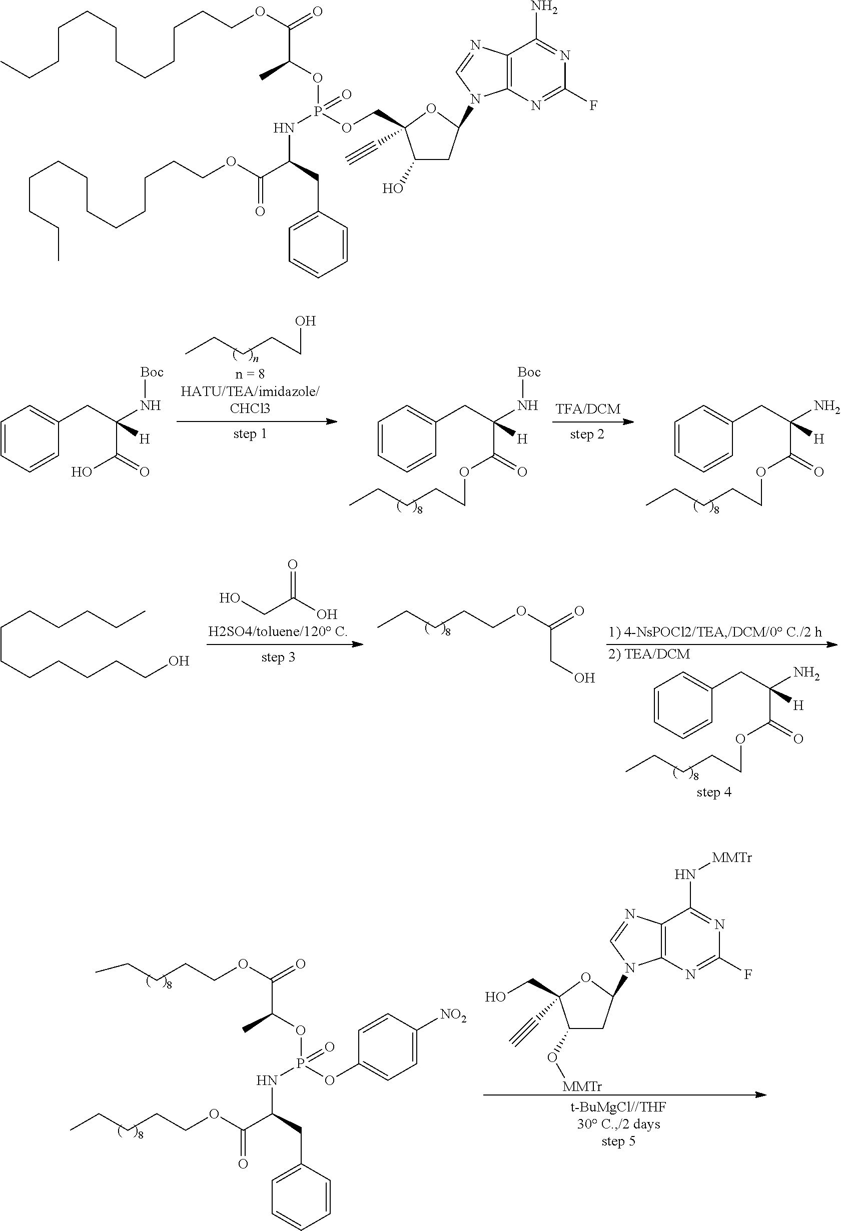

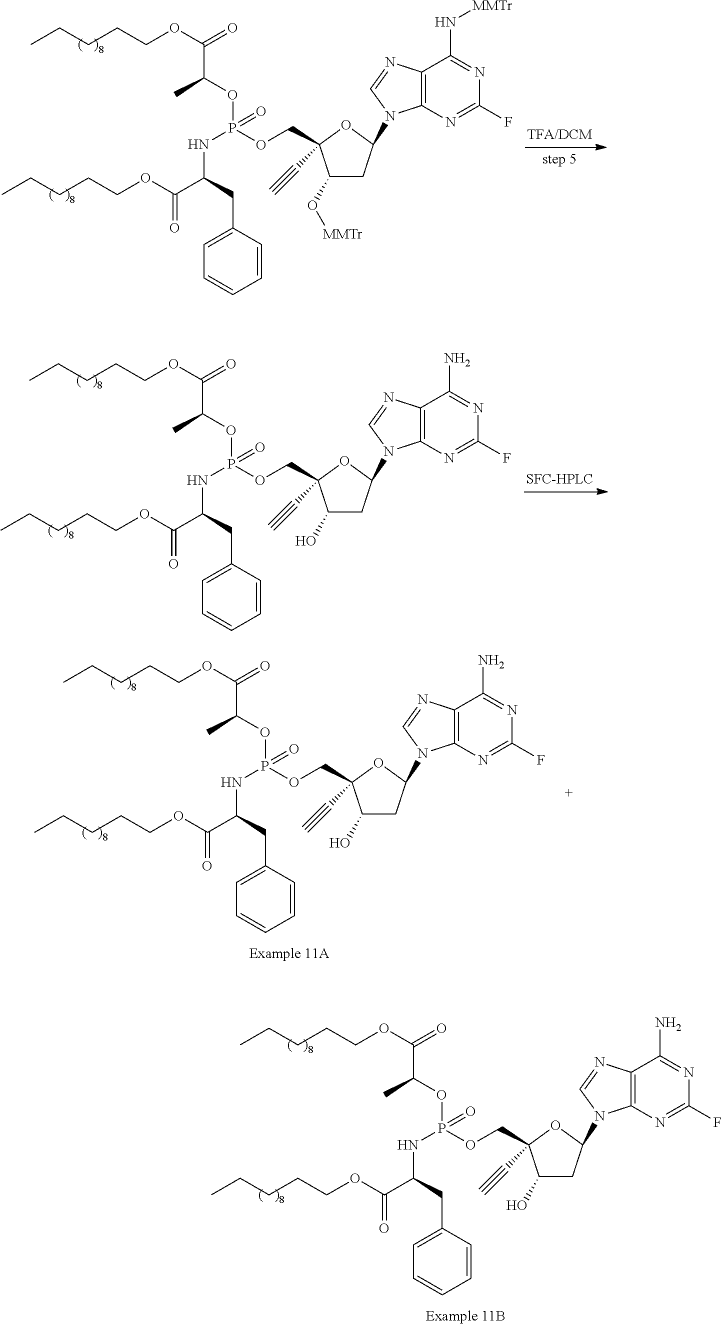

### Step 1: Dodecyl (tert-butoxycarbonyl)-L-phenylalaninate

To a solution of (tert-butoxycarbonyl)-L-phenylalanine (15 g, 56.5 mmol) and HATU (43.0 g, 113 mmol) in chloroform (100 mL) stirred at room temperature was added dodecan-1-ol (15.80 g, 85 mmol), 1H-imidazole (11.55 g, 170 mmol) and N-ethyl-N-isopropylpropan-2-amine (30.2 mL, 170 mmol). The reaction mixture was stirred at room temperature for 18 hours. LCMS indicated completion of. reaction. The reaction mixture was quenched with water and extracted with dichloromethane (150 mL). The organic phase was washed with saturated brine (80 mL), dried over sodium sulphate and evaporated under vacuum to give the crude product as yellow oil. The crude product was purified by gel silica column (330 g, pet. ether:EtOAc=4:1) to give dodecyl (tert-butoxycarbonyl)-L-phenylalaninate (16 g, 35.8 mmol, 63.3% yield) as colourless oil. LCMS (ESI) m/z calcd for C₂₆H₄₃NO₄: 433; found: 378 (M+H−56). ¹H NMR (400 MHz, Chloroform-d) δ 7.32-7.26 (m, 2H), 7.26-7.20 (m, 1H), 7.16-7.11 (m, 2H), 4.98 (d, J=8.4 Hz, 1H), 4.61-4.53 (m, 1H), 4.11-4.04 (m, 2H), 3.14-3.01 (m, 2H), 1.58 (t, J=6.8 Hz, 2H), 1.42 (s, 9H), 1.34-1.21 (m, 18H), 0.91-0.84 (m, 3H).

### Step 2: Dodecyl L-phenylalaninate

To a solution of dodecyl (tert-butoxycarbonyl)-L-phenylalaninate (16 g, 36.9 mmol) in dichloromethane (160 mL) stirred at room temperature was added TFA (30 mL, 389 mmol) and stirred for 1 hour. LCMS indicated completion of. reaction. The reaction mixture was quenched with saturated aqueous NaHCO₃ and extracted with dichloromethane (100 mL). The organic phase was washed with saturated brine (50 mL), dried over sodium sulphate and evaporated under vacuum to give dodecyl L-phenylalaninate (12 g, 35.6 mmol, 97% yield) as yellow solid. LCMS (ESI) m/z calcd for C₂₁H₃₅NO₂: 333; found: 334 (M+H). ¹H NMR (400 MHz, Chloroform-d) δ 7.33-7.26 (m, 2H), 7.26-7.18 (m, 3H), 4.09 (d, J=6.4 Hz, 2H), 3.74 (dd, J=7.6, 5.2 Hz, 1H), 3.09 (dd, J=13.6, 5.6 Hz, 1H), 2.89 (dd, J=13.6, 7.6 Hz, 1H), 1.87 (s, 2H), 1.64-1.54 (m, 2H), 1.30-1.24 (m, 16H), 0.92-0.85 (m, 3H).

### Step 3: Dodecyl 2-hydroxyacetate

To a solution of 2-hydroxyacetic acid (5 g, 65.7 mmol) and dodecan-1-ol (24.50 g, 131 mmol) in toluene (50 mL) stirred under nitrogen was added sulfuric acid (1 mL, 65.7 mmol) at room temperature. The reaction mixture was stirred at 120° C. for 5 hours. LCMS indicated completion of. reaction. The reaction mixture was evaporated and the residue was extracted with ethyl acetate (50 mL). The organic phase was washed with water (30 mL) and saturated sodium bicarbonate solution (30 mL), dried over sodium sulphate and evaporated under vacuum to give crude dodecyl 2-hydroxyacetate (20 g, 49.1 mmol, 74.7% yield) as white solid. ¹H NMR (400 MHz, DMSO-d₆) δ 5.28 (t, J=6.4 Hz, 1H), 4.02 (t, J=6.8 Hz, 2H), 3.96 (d, J=6.4 Hz, 2H), 1.59-1.48 (m, 2H), 1.48-1.33 (m, 2H), 1.29-1.19 (m, 16H), 0.89-0.79 (m, 3H).

### Step 4: Dodecyl ((2-(dodecyloxy)-2-oxoethoxy)(4-nitrophenoxy)phosphoryl)-L-phenylalaninate

To a solution of 4-nitrophenyl phosphorodichloridate (4.19 g, 16.37 mmol) and dodecyl 2-hydroxyacetate (4 g, 16.37 mmol) in dichloromethane (100 mL) stirred under nitrogen at 0° C. was added triethylamine (2.510 mL, 18.01 mmol) dropwise. The reaction mixture was stirred at 0° C. for 2 hours. To the above mixture was added dodecyl L-phenylalaninate (5.46 g, 16.37 mmol) in dichloromethane (50 mL) and triethylamine (5.02 mL, 36.0 mmol) dropwise at 0° C. under nitrogen. The reaction mixture was stirred at 0° C. for 1 hour. LCMS indicated completion of. reaction. The reaction mixture was quenched with water, extracted with dichloromethane (80 mL). The organic phase was washed with water (50 mL) and saturated brine (50 mL), dried over sodium sulphate and evaporated under vacuum to give the crude product as yellow oil. The crude product was purified by gel silica column (330 g, pet. ether:EtOAc=10:1) to give the product (5 g) as yellow oil. The yellow oil was further purified by reverse phase column (330 g, water-5 mM NH₄CO₃/acetonitrile) twice to give dodecyl ((2-(dodecyloxy)-2-oxoethoxy)(4-nitrophenoxy)phospho-ryl)-L-phenylalaninate (3.5 g, 4.14 mmol, 25.3% yield) as colourless oil. LCMS (ESI) m/z calcd for C₄₁H₆₅N₂O₉P: 760; found: 761 (M+H). ¹H NMR (400 MHz, Chloroform-d) δ 8.21-8.14 (m, 2H), 7.33-7.24 (m, 5H), 7.17-7.10 (m, 2H), 4.50-4.27 (m, 3H), 4.20-4.11 (m, 2H), 4.11-4.04 (m, 2H), 3.72-3.61 (m, 1H), 3.15-3.05 (m, 1H), 3.01-2.91 (m, 1H), 1.69-1.60 (m, 2H), 1.26 (br s, 36H), 0.93-0.82 (m, 6H).

### Step 5: Dodecyl ((2-(dodecyloxy)-2-oxoethoxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate

To a mixture of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (700 mg×2, 0.835 mmol) in tetrahydrofuran (7 mL) was added tert-butylmagnesium chloride/THF (1.253 mL×2, 1.253 mmol) at 0° C. under nitrogen atmosphere. The reaction mixture was stirred for 1 hour at room temperature. To the reaction mixture was added dodecyl ((2-(dodecyloxy)-2-oxoethoxy)(4-nitrophenoxy)-phosphoryl)-L-phenylalaninate (1271 mg×2, 1.671 mmol) at 0° C. The reaction mixture was stirred for 12 hours at room temperature. LCMS indicated completion of. reaction. The reaction mixture was purified by gel silica column (120 g, pet. ether:EtOAc=5:1) to give dodecyl ((2-(dodecyloxy)-2-oxoethoxy)(((2R,3S,5R)-2-ethynyl-5-(2-fluoro-6-(((4-methoxy-phenyl)diphenylmethyl)amino)-9H-purin-9-yl)-3-((4-methoxyphenyl)diphenylmethoxy)-tetrahydrofuran-2-yl)methoxy)phosphoryl)-L-phenylalaninate (2 g, 1.233 mmol, 73.8 yield) as yellow solid. LCMS (ESI) m/z calcd for C₈₇H₁₀₄FN₆O₁₁P: 1459; found: 1460 (M+H).

### Step 6: Dodecyl ((((2R,3S,5R)-5-(6-amino-2-fluoro-9H-purin-9-yl)-2-ethynyl hydroxytetrahydrofuran-2-yl)methoxy)(2-(dodecyloxy)-2-oxoethoxy)phosphoryl)-L-phenylalaninate

To a solution of dodecyl ((2-(dodecyloxy)-2-oxoethoxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate (2 g, 1.370 mmol) in DCM (20 mL) stirred at room temperature was added TFA (2 ml, 26.0 mmol) dropwise. The reaction mixture was stirred at room temperature for 1 hour. LCMS indicated completion of reaction. The reaction mixture was quenched with water and extracted with dichloromethane (20 mL). The organic phase was washed with saturated NaHCO₃ solution (20 mL) and saturated brine (20 mL), dried over sodium sulphate and evaporated under vacuum to give the crude product as yellow oil. The crude product was purified by silica gel column (120 g, DCM: MeOH=10:1) to give the title compound (1 g) as yellow oil. The isomers were separated by SCF (Column: CHIRALPAK AS-H, 2.0*25 cm L (5 μm); Mobile Phase A: CO2, Mobile Phase B: IPA; Flow rate: 80 mL/min; Gradient: 50% B; 220 nm; RT1: 3.01; RT2: 4.45; Injection Volumn: 2 ml; number of runs: 11; to give first eluting isomer (Example 11A, 309.5 mg, RT: 3.01) as white solid. LCMS (ESI) m/z calcd for C₄₇H₇₂FN₆O₉P: 914; found: 915 (M+H). ¹H NMR (400 MHz, Chloroform-d) δ 8.17 (s, 1H), 7.32-7.14 (m, 5H), 6.38-6.31 (m, 1H), 6.13 (s, 2H), 4.86 (t, J=8.0 Hz, 1H), 4.39 (dd, J=16.4, 10.0 Hz, 1H), 4.31-3.98 (m, 9H), 3.55-3.44 (m, 1H), 3.19-3.10 (m, 1H), 2.93 (dd, J=13.6, 8.0 Hz, 1H), 2.79-2.70 (m, 3H), 1.66-1.56 (m, 4H), 1.34-1.22 (m, 36H), 0.92-0.84 (m, 6H); and the second eluting isomer (Example 11B, 265.9 mg, RT: 4.45) as white solid. LCMS (ESI) m/z calcd for C₄₇H72FN₆O₉P: 914; found: 915 (M+H). ¹H NMR (400 MHz, Chloroform-d) δ 8.07 (s, 1H), 7.29-7.14 (m, 5H), 6.39-6.11 (m, 3H), 4.71 (t, J=7.6 Hz, 1H), 4.41 (dd, J=16.0, 10.4 Hz, 1H), 4.26-4.04 (m, 8H), 3.83-3.73 (m, 2H), 3.16-3.06 (m, 1H), 2.93 (dd, J=14.0, 8.0 Hz, 1H), 2.75-2.62 (m, 3H), 1.68-1.53 (m, 4H), 1.34-1.22 (m, 36H), 0.92-0.84 (m, 6H).

### Example 12

### Dodecyl ((((2R,3S,5R)-5-(6-amino-2-fluoro-9H-purin-9-yl)-2-ethynyl-3-hydroxytetrahydrofuran-2-yl)methoxy)(((S)-1-(dodecyloxy)-1-oxopropan-2-yl)oxy)phosphoryl)-L-phenylalaninate

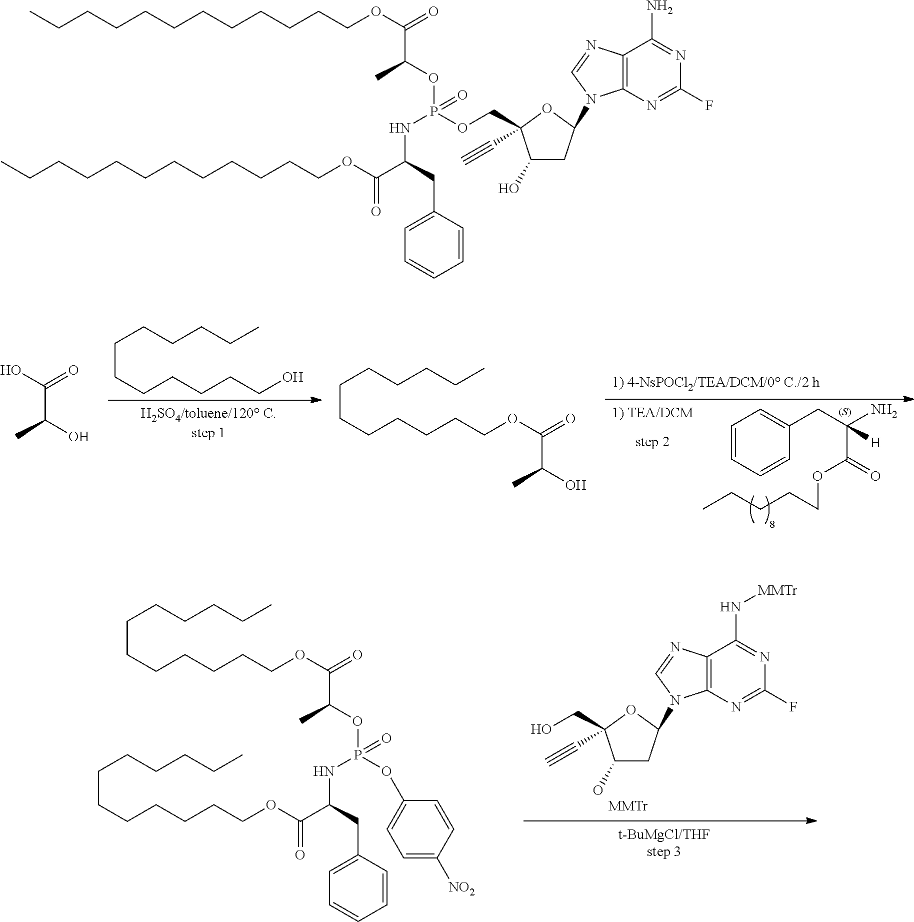

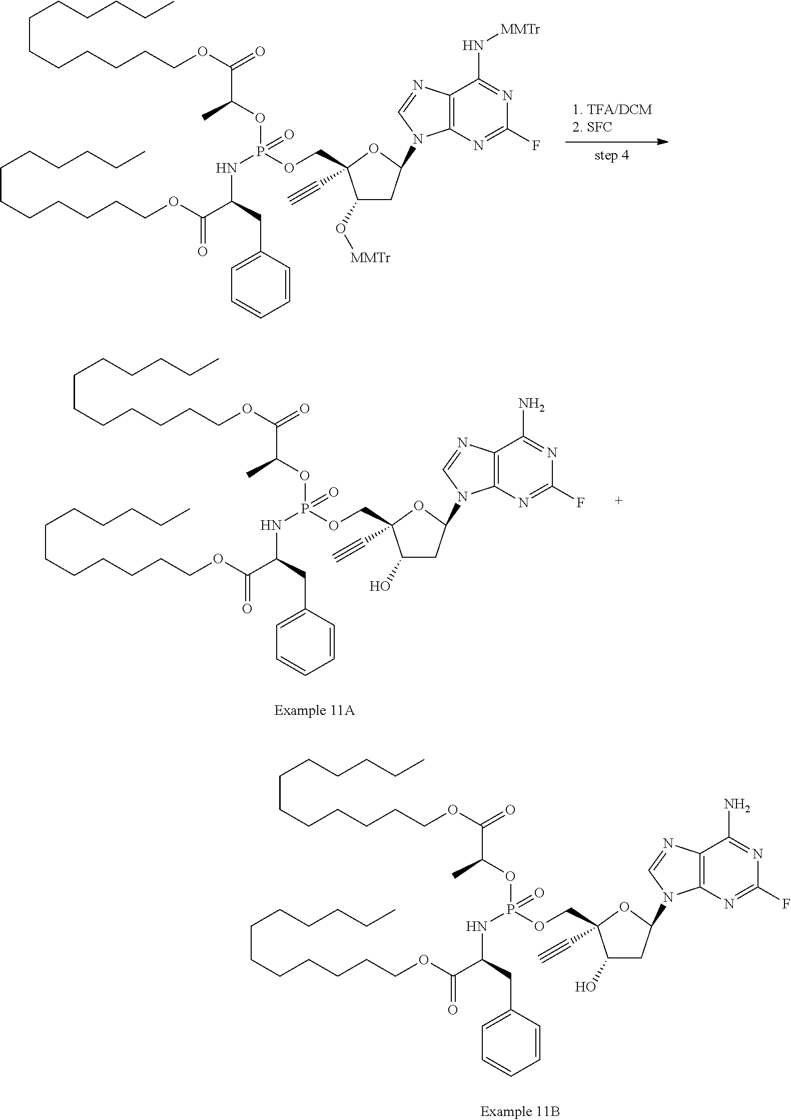

### Step 1: Dodecyl (S)-2-hydroxypropanoate

To a solution of (S)-2-hydroxypropanoic acid (5 g, 55.5 mmol) and dodecan-1-ol (20.69 g, 111 mmol) in toluene (50 mL) stirred under nitrogen was added H2SO4 (0.5 mL, 9.38 mmol) at room temperature. The reaction mixture was stirred at 120° C. for 16 hr. Then, the reaction mixture was concentrated and the residue extracted with ethyl acetate (50 mL). The organic phase was washed with water (20 mL) and saturated NaHCO₃ solution (20 mL), dried over sodium sulphate and evaporated under vacuum to give the crude product as yellow oil. The crude product was crystallized from pet. ether (50 mL) to afford dodecyl (S)-2-hydroxypropanoate (10 g, 23.22 mmol, 41.8% yield) as white solid. ¹H NMR (400 MHz, Chloroform-d) δ 4.28-4.21 (m, 1H), 4.21-4.10 (m, 2H), 1.68-1.59 (m, 2H), 1.58-1.50 (m, 2H), 1.39 (d, J=6.8 Hz, 3H), 1.36-1.19 (m, 16H), 0.90-0.81 (m, 3H).

### Step 2: Dodecyl ((((S)-1-(dodecyloxy)-1-oxopropan-2-yl)oxy)(4-nitrophenoxy)phosphoryl)-L-phenylalaninate

To a stirred mixture of 4-nitrophenyl phosphorodichloridate (2.1 g, 8.20 mmol) in DCM (20 mL) was added dodecyl (S)-2-hydroxypropanoate (2.120 g, 8.20 mmol) and TEA (830 mg) at room temperature. The reaction mixture was stirred at room temperature for 2 h. To the above mixture was added a solution of dodecyl L-phenylalaninate (2.189 g, 6.56 mmol) in DCM (3 ml) and TEA (6.86 mL, 49.2 mmol). The reaction mixture was stirred at room temperature for 16 hr. LCMS analysis showed presence of desired product. The reaction mixture was quenched with water (50 mL) and extracted with DCM (3*50 mL). The combined organic layers were washed with brine and dried over Na2SO4. After filtration, the filtrate was concentrated to dryness under vacuum. The residue was purified by silica gel column (120 g, pet. ether:EtOAc=1:4) to afford dodecyl ((((S)-1-(dodecyloxy) oxopropan-2-yl)oxy)(4-nitrophenoxy)phosphoryl)-L-phenylalaninate (1.2 g, 1.548 mmol, 18.87% yield) as yellow oil. LCMS (ESI) m/z calcd for C₄₂H₆₇N₂O₉P: 773; found: 774 (M+1).

### Step 3: Dodecyl ((((S)-1-(dodecyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate

To a solution of ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (800 mg, 0.955 mmol) in THF (4 mL) stirred under nitrogen was added a solution of tert-butylmagnesium chloride/THF (1.432 mL, 1.432 mmol) at room temperature. The reaction mixture was stirred at 25° C. for 1 hour. Then, the decyl dodecyl ((((S)-1-(dodecyloxy)-1-oxopropan-2-yl)oxy)(4-nitrophenoxy)phosphoryl)-L-phenylalaninate (1110 mg, 1.432 mmol) was added to the reaction mixture at 25° C. The resulting mixture was stirred at room temperature for 6 hour. The reaction mixture was concentrated to dryness under vacuum and the residue was purified by silica gel column (120 g, pet. ether:EtOAc=1:1) to afford dodecyl ((((S)-1-(dodecyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate (1.0 g, 0.339 mmol, 35.5% yield). as yellow solid. LCMS (ESI) m/z calcd for C₈₈H₁₀₆FN₆O₁₁P: 1473; found: 1474 (M+1).

### Step 4: Dodecyl ((((2R,3S,5R)-5-(6-amino-2-fluoro-9H-purin-9-yl)-2-ethynyl-3-hydroxytetrahydrofuran-2-yl)methoxy)(((S)-1-(dodecyloxy)-1-oxopropan-2-yl)oxy)phosphoryl)-L-phenylalaninate

To a stirred mixture of dodecyl (MS)-1-(dodecyloxy)-1-oxopropan-2-yl)oxy)(((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)phosphoryl)-L-phenylalaninate (900 mg, 0.397 mmol) in DCM (9 mL) was added TFA (0.9 mL) at 0° C. The reaction mixture was stirred for 2 h at room temperature. Then, the reaction mixture was concentrated to dryness under vacuum and the residue was purified by silica gel column (80 g, MeOH:DCM=1:19) to afford dodecyl ((((2R,3S,5R)-5-(6-amino-2-fluoro-9H-purin-9-yl)-2-ethynyl-3-hydroxytetrahydrofuran-2-yl)methoxy)(((S)-1-(dodecyloxy) oxopropan-2-yl)oxy)phosphoryl)-L-phenylalaninate (300 mg) as a yellow oil. The isomers were separated by following condition:Column: CHIRALPAK IH, 2.0*25 cm, 5 μm; Mobile Phase A: CO2, Mobile Phase B: MEOH (2 mM NH3-MEOH); Flow rate: 40 mL/min; Gradient: 30% B; 220 nm; RT1: 2.98; RT2: 4.49; Injection Volumn: 1.5 ml; number of runs: 15; the fractions containing desired products were concentrated to dryness under vacuum. The first eluting isome (RT1: 2.98) was re-crystallized from ACN/H₂O in the ratio of 1:1. The solid was collected by filtration and dried under sun lamp (45° C.) to give Example 12A as white crystal solid. LCMS (ESI) m/z calcd for C₄₈H₇₄FN₆O₉P: 929; found: 930 (m+1). ¹H NMR (300 MHz, CDCl₃) δ 8.10 (s, 1H), 7.26-7.16 (m, 5H), 6.36 (s, 1H), 6.28-5.82 (m, 1H), 4.73 (s, 2H), 4.21-4.07 (m, 7H), 3.60-3.50 (m, 1H), 3.15-2.95 (m, 2H), 2.90-2.54 (m, 3H), 2.45-1.75 (m, 2H), 1.60 (t, J=6 Hz 4H), 1.45-1.18 (m, 39H), 0.88 (t, J=6 Hz 6H); and the second eluting isomer (RT2: 4.49) was re-crystallized from ACN/H₂O. The solid was collected by filtration and dried under sun lamp (45° C.) to afford Example 12B as white amorphous solid. LCMS (ESI) m/z calcd for C₄₈H₇₄FN₆O₉P: 929; found: 930 (m+1). ¹H NMR (300 MHz, CDCl₃) δ 8.20 (s, 1H), 7.26-7.15 (m, 5H), 6.35 (t, J=6 Hz, 1H), 6.09 (s, 1H), 5.30 (s, 1H), 4.56 (t, J=9 Hz, 1H), 4.60-4.48 (m, 1H), 4.20-4.08 (m, 6H), 3.48 (s, 1H), 3.25-2.92 (m, 2H), 2.73 (t, J=3 Hz 3H), 1.70-1.53 (m, 3H), 1.49 (d, J=3 Hz, 2H), 1.26 (br s, 39H), 0.88 (t, J=6 Hz, 6H).

### Example 13

### Decyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

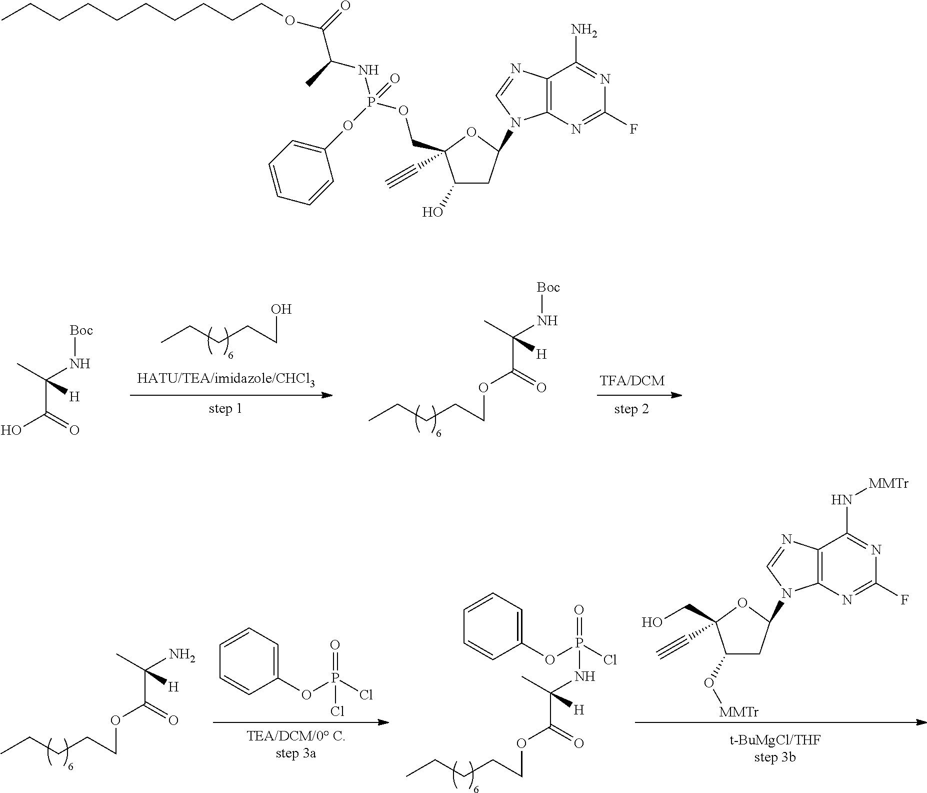

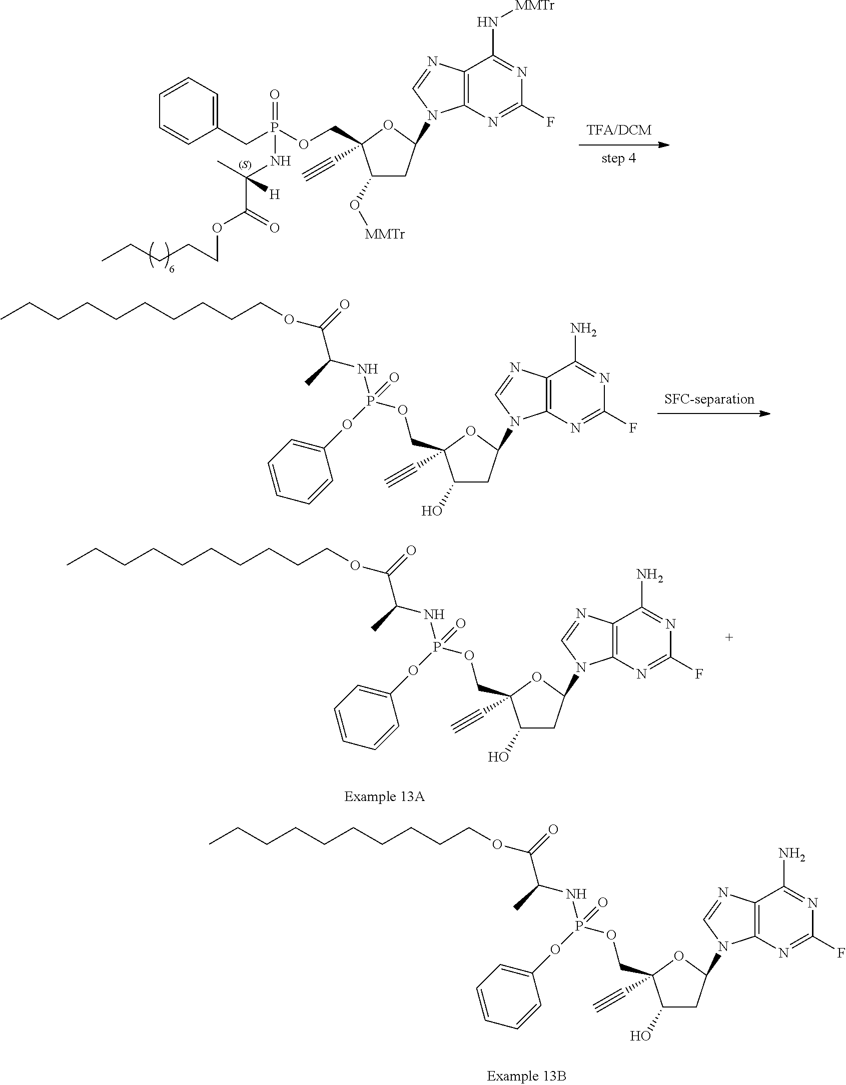

### Step 1: Decyl (tert-butoxycarbonyl)-L-alaninate

To a stirred mixture of (tert-butoxycarbonyl)-L-alanine (10 g, 52.9 mmol) in chloroform (100 mL) was added HATU (30.1 g, 79 mmol), decan-1-ol (12.55 g, 79 mmol), TEA (22.10 mL, 159 mmol) and imidazole (10.79 g, 159 mmol) at 0° C. The reaction mixture was stirred for 2 h at room temperature. TLC showed the reaction was completed (stained with KMnO4, pet. ether:EtOAc=2:1). The reaction was diluted with water (100 mL), extracted with DCM (2×100 mL) and the combined organic extracts were washed with saturated aqueous NaHCO₃ (2×100 mL) and brine (50 mL) and concentrated to dryness under vacuum. The residue was purified by silica gel column (330 g, pet. ether:EtOAc=2:1) to afford decyl (tert-butoxycarbonyl)-L-alaninate (9 g, 24.58 mmol, 46.5% yield) as a yellow oil. ¹H NMR (400 MHz, DMSO-d6) δ 7.25 (d, J=7.4 Hz, 1H), 4.10-3.86 (m, 3H), 1.64-1.48 (m, 2H), 1.39-1.18 (m, 26H), 0.89-0.78 (m, 3H).

### Step 2: Decyl L-alaninate

To a solution of. decyl (tert-butoxycarbonyl)-L-alaninate (8.5 g, 25.8 mmol) in DCM (85 mL) stirred under nitrogen at 0° C. was added TFA (17 ml, 221 mmol). The reaction mixture was stirred at 0° C. for overnight. LCMS indicated completion of. reaction. The reaction mixture was quenched with water, aqueous layer separated and the organic phase was washed with water (100 mL), dried over sodium sulphate and concentrated under vacuum to give the crude product as yellow oil. The crude product was added to a gel silica column (330 g) and was eluted with 0-10% EtOAc/Hex. The fraction containing desired product concentrated under vacuum to give decyl L-alaninate (6.2 g, 27.0 mmol, 105% yield) as yellow oil. LCMS (ESI) m/z calcd for C13H27NO2: 229; found: 230. ¹H NMR (400 MHz, Chloroform-d) δ 4.16-4.06 (m, 2H), 3.56-3.51 (m, 1H), 1.68-1.61 (m, 2H), 1.29 (s, 17H), 0.91-0.85 (m, 3H).

### Step 3: Decyl((((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methoxy)(phenoxy)phosphoryl)-L-alaninate

To a solution of. decyl L-alaninate (3 g, 13.08 mmol), TEA (1.823 mL, 13.08 mmol) and phenyl phosphorodichloridate (2.76 g, 13.08 mmol) in DCM (30 mL) stirred under nitrogen at room temperature for 1 h. Then a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmeth-oxy)tetrahydrofuran-2-yl)methanol (1.5 g, 1.790 mmol) and 1M tert-butylmagnesium chloride/THF (18.31 mL, 18.31 mmol) in THF (15 mL)/pyridine (15.0 mL) was stirred at room temperature for 30 min was added to above mixture. The reaction mixture was stirred at room temperature overnight. LCMS indicated completion of. reaction. The reaction mixture was quenched with water, partitioned between dichloromethane (100 mL) and water (100 mL). The organic phase was washed with water (100 mL) and saturated brine (100 mL), dried. over sodium sulphate and evaporated under vacuum to give the crude product as white solid. The residue was purified by gel silica column (330 g, pet. ether:EtOAc=1:1) to give decyl((((2R,3S,5R)-2-ethynyl-5-(2-fluoro-6-(((4-methoxyphen-yl)diphenylmethyl)amino)-9H-purin-9-yl)-3-((4-methoxyphenyl)diphenylmethoxy)tetrahydrofuran-2-yl)methoxy)(phenoxy)phosphor-yl)-L-alaninate (1 g, purity: 93.9%, yield: 5.96%) as white solid. LCMS (ESI) m/z calcd for C₇₁H₇₄FN₆O₉P: 1204; found: 1205. ¹H NMR (300 MHz, Chloroform-d) δ 7.80-7.68 (m, 1H), 7.62-7.51 (m, 4H), 7.47-7.02 (m, 25H), 6.92-6.73 (m, 4H), 6.25-6.06 (m, 1H), 4.65-4.53 (m, 1H), 4.38-4.28 (m, 2H), 4.20-4.02 (m, 4H), 3.82-3.72 (m, 6H), 3.57-3.46 (m, 1H), 2.89-2.83 (m, 1H), 2.04-1.94 (m, 1H), 1.92-1.84 (m, 1H), 1.78-1.69 (m, 2H), 1.51-1.41 (m, 2H), 1.32-1.24 (m, 15H), 0.90 (t, J=6.0 Hz, 3H).

### Step 4: Decyl((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of. decyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(phenoxy)phosphoryl)-L-alaninate (1 g, 0.830 mmol) in DCM (10 mL) stirred under nitrogen was added. TFA (1 mL, 12.98 mmol) at room temperature. The reaction mixture was stirred at room temperature for 1 h. LCMS indicated completion of. reaction. The reaction mixture was quenched with water and extracted with DCM (20 mL). The organic phase was washed with saturated sodium carbonate (25 mL), water (25 mL) and saturated brine (25 mL), dried over sodium sulphate and evaporated under vacuum to give the crude product as white solid. The crude product was purified by Prep-SFC Column: CHIRALPAK IA, 2*25 cm, 5 μm; Mobile Phase A: Hex: DCM=3:1 (0.1% FA)-HPLC, Mobile Phase B: EtOH-HPLC; Flow rate: 15 mL/min; Gradient: 30 B to 30 B in 10 min; 220/254 nm; RT1: 5.582; RT2: 7.933; Injection Volumn: 1 ml; number of runs: 6 to give first eluting isomer Example 13A (RT1: 5.582) was 100 mg as white solid. (The solid repurified by A-Chiral Column: DAICEL DCpak P4VP, 20*250 cm, 5 um; Mobile Phase A: CO2, Mobile Phase B: MeOH (8 mmol/L NH3/MeOH)-HPLC; Flow rate: 50 mL/min; Gradient: 20% B; 254 nm; Injection Volumn: 1.5 ml; number of runs: 4; to give first eluting isomer (Example 13A, RT1: 6.63, 60.8 mg) as white solid. LCMS (ESI) m/z calcd for C₃₁H₄₂FN₆O₇P: 660; found: 661 (M+H). ¹H NMR (400 MHz, DMSO-d6) δ 8.25 (s, 1H), 7.85 (s, 2H), 7.35-7.26 (m, 2H), 7.18-7.09 (m, 3H), 6.28 (dd, J=7.6, 4.8 Hz, 1H), 6.02-6.00 (m, 1H), 5.83 (d, J=5.2 Hz, 1H), 4.66-4.64 (m, 1H), 4.21 (dd, J=10.8, 5.6 Hz, 1H), 4.10 (dd, J=11.2, 4.4 Hz, 1H), 3.98-3.89 (m, 2H), 3.72-3.64 (m, 2H), 2.83-2.74 (m, 1H), 1.49-1.44 (m, 2H), 1.28-1.08 (m, 17H), 0.89-0.81 (m, 3H); and second eluting isomer (Example 13B, R.T: 7.933, 104.9 mg) as white solid. LCMS (ESI) m/z calcd for C₃₁H₄₂FN₆O₇P: 660; found: 661 (M+H). ¹H NMR (300 MHz, DMSO-d6) δ 8.27 (s, 1H), 7.85 (s, 2H), 7.38-7.26 (m, 2H), 7.22-7.08 (m, 3H), 6.28-6.24 (m, 1H), 6.03-6.00 (m, 1H), 5.86-5.73 (m, 1H), 4.66-4.56 (m, 1H), 4.24 (dd, J=10.5, 5.7 Hz, 1H), 4.06 (dd, J=11.1, 4.8 Hz, 1H), 3.99-3.71 (m, 3H), 3.64 (s, 1H), 2.83-2.67 (m, 1H), 1.53-1.37 (m, 2H), 1.31-1.11 (m, 17H), 0.89-0.80 (m, 3H).

### Example 14

Dodecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

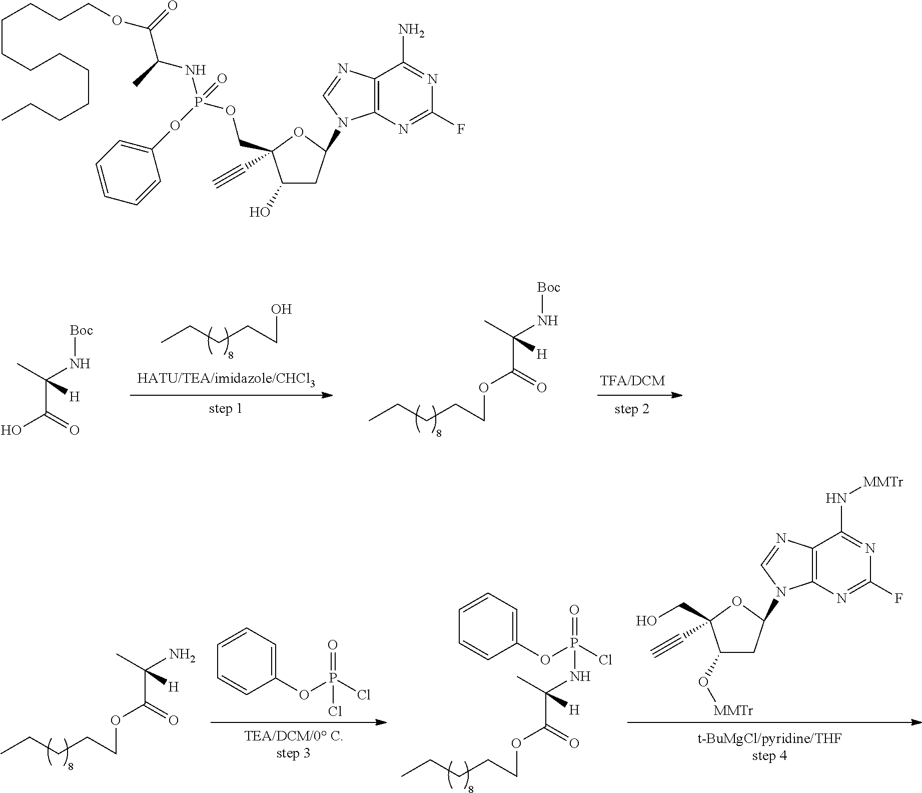

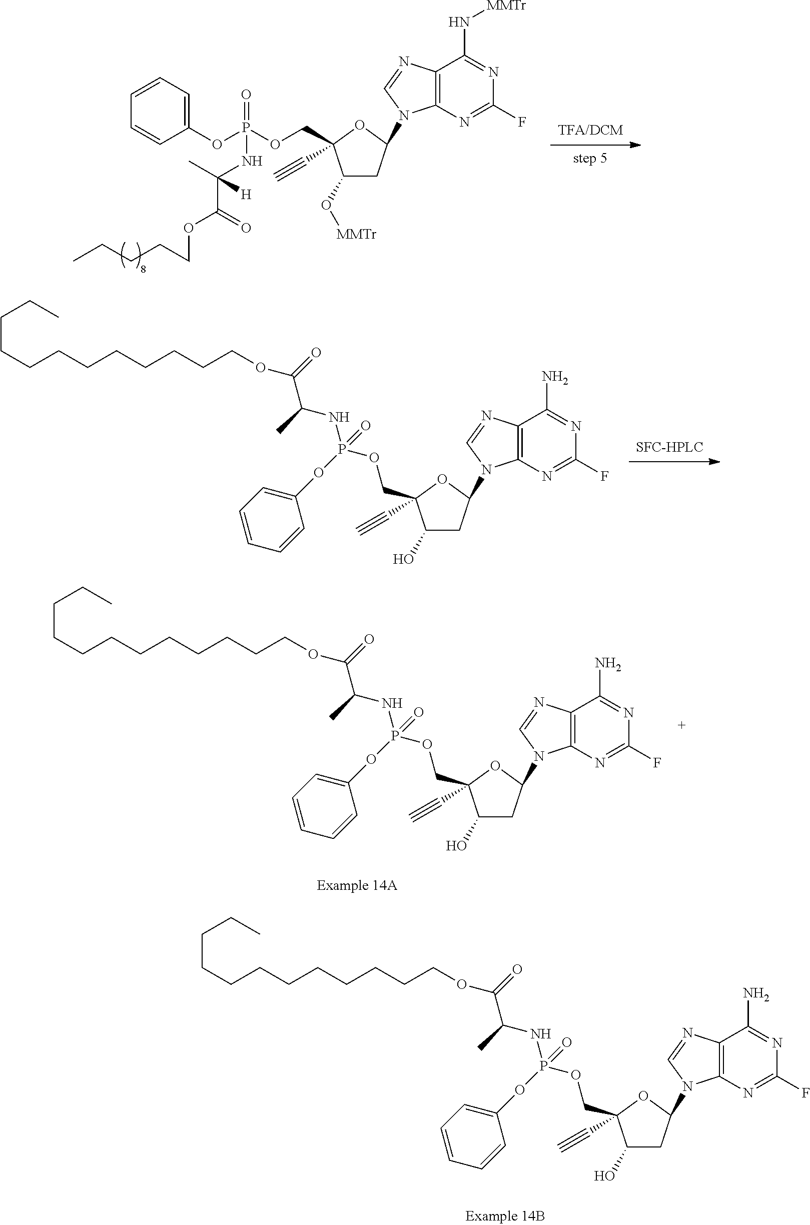

### Step 1: Dodecyl (tert-butoxycarbonyl)-L-alaninate

To a stirred mixture of (tert-butoxycarbonyl)-L-alanine (10 g, 52.9 mmol) in chloroform (100 mL) was added HATU (30.1 g, 79 mmol), dodecan-1-ol (14.77 g, 79 mmol), TEA (22.10 mL, 159 mmol) and imidazole (10.79 g, 159 mmol) at 0° C. The reaction mixture was stirred for 5 h at room temperature. TLC showed the reaction was completed (stained with KMnO4, pet. ether:EtOAc=2:1). The reaction was diluted with water (100 mL), extracted with DCM (2×100 mL) and the combined organic extracts were washed with saturated aqueous NaHCO₃ (2×100 mL) and brine (50 mL) and concentrated to dryness under vacuum. The residue was purified by silica gel column (330 g, pet. ether:EtOAc=2:1) to afford dodecyl (tert-butoxycarbonyl)-L-alaninate (6 g, 28.6% yield) as yellow oil. ¹H NMR (300 MHz, Chloroform-d) δ 5.06 (d, J=6.0 Hz, 1H), 4.28 (t, J=7.1 Hz, 1H), 4.20-4.03 (m, 2H), 1.72-1.55 (m, 2H), 1.43 (s, 9H), 1.39-1.30 (m, 5H), 1.25 (br s, 16H), 0.96-0.78 (m, 3H).

### Step 2: Dodecyl L-alaninate

To a solution of. dodecyl (tert-butoxycarbonyl)-L-alaninate (5.5 g, 15.38 mmol). in DCM (55 mL) stirred under nitrogen added TFA (11 ml, 143 mmol) and stirred at 0° C. for 1 h. LCMS indicated completion of. reaction. The reaction mixture was quenched with water and extracted with DCM (50 mL). The organic phase was washed with water (100 mL), dried over sodium sulphate and evaporated under vacuum to give the crude product as yellow oil. The residue was purified by gel silica column (330 g, pet. ether:EtOAc=20:1) to give dodecyl L-alaninate (4.0 g, purity: 100%; yield 100%) as yellow oil. LCMS (ESI) m/z calcd for C15H31NO2: 257; found: 258 (M+1). ¹H NMR (400 MHz, Chloroform-d): δ 4.22-3.98 (m, 2H), 3.59-3.46 (m, 1H), 1.74-1.58 (m, 2H), 1.32 (s, 3H), 1.29 (br s, 18H), 0.92-0.87 (m, 3H).

### Step 3: Decyl ((((2R,3S,5R)-2-ethynyl-5-(2-fluoro-6-(((4-methoxyphenyl)diphenylmethyl)-amino)-9H-purin-9-yl)-3-(4-methoxyphenyl)diphenylmethoxy)tetrahydrofuran-2-yl)methoxy)(phenoxy)phosphoryl)-L-alaninate

To a solution of. dodecyl L-alaninate (3 g, 11.65 mmol), TEA (4.87 mL, 35.0 mmol) and phenyl phosphorodichloridate (2.459 g, 11.65 mmol) in DCM (30 mL) stirred under nitrogen at room temperature and stirred for 1 h. Then, a solution of ((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)-tetrahydrofuran-2-yl)methanol (1.5 g, 1.790 mmol) and tert-butylmagnesium chloride/THF (16.32 mL, 16.32 mmol) in THF (15 mL)/pyridine (15 mL) was stirred at room temperature for 30 min was added to above mixture. The resulting reaction mixture was stirred at room temperature overnight. The reaction mixture was quenched with water, and partitioned between dichloromethane (100 mL) and water (100 mL). The organic phase was washed with water (100 mL) and saturated brine (100 mL), dried. over sodium sulphate and evaporated under vacuum to give the crude product as white solid. The residue was purified by gel silica column (330 g, pet. ether:EtOAc=1:1) to give dodecyl ((((2R,3S,5R)-2-ethynyl-5-(2-fluoro-6-(((4-methoxyphen-yl)diphenylmethyl) amino)-9H-purin-9-yl)-3-((4-methoxyphenyl)diphenylmethoxy)tetrahydrofuran-2-yl)methoxy)(phenoxy)phosphoryl)-L-alaninate (1.8 g, purity: 98%, yield: 12.27%) as white solid. LCMS (ESI) m/z calcd for C₇₃H78FN₆O₉P: 1233; found: 1234 (M+1). ¹H NMR (300 MHz, Chloroform-d) δ 7.79-7.71 (m, 1H), 7.60-7.49 (m, 4H), 7.45-7.04 (m, 25H), 6.90-6.74 (m, 4H), 6.21-6.13 (m, 1H), 4.65-4.53 (m, 1H), 4.33 (t, J=6.6 Hz, 2H), 4.26-4.17 (m, 1H), 4.15-4.00 (m, 3H), 3.84-3.71 (m, 6H), 3.59-3.42 (m, 1H), 2.05-1.97 (m, 1H), 1.75 (dd, J=8.7, 6.3 Hz, 2H), 1.54-1.41 (m, 2H), 1.27 (br s, 19H), 0.93-0.86 (m, 3H).

### Step 4

### Dodecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of. dodecyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran yl)methoxy)(phenoxy)phosphoryl)-L-alaninate (1.8 g, 1.459 mmol) in DCM (18 mL) stirred under nitrogen at room temperature was added. TFA (1.8 mL, 23.36 mmol). The reaction mixture was stirred at room temperature for 1 h. LCMS indicated completion of reaction. The reaction mixture was quenched with water, extracted with dichloromethane (20 mL). The organic phase was washed with saturated sodium bicarbonate solution (20 mL), water (25 mL) and saturated brine (25 mL), dried over sodium sulphate and evaporated under vacuum to give the crude product as white solid. The crude product was purified by Prep-SFC Column: CHIRALPAK IH, 2*25 cm, 5 μm; Mobile Phase A: CO2, Mobile Phase B: MEOH (2 mM NH3-MEOH); Flow rate: 5 mL/min; Gradient: 30% B; 220 nm; RT1: 3.94; RT2: 5.13; Injection Volumn: 1.5 ml; number of runs: 14; to give the first eluting isomer Example 14A (RT: 3.94) was 300 mg, as a white solid which was further purified by Prep-Achiral Column:Column: DAICEL DCpak P4VP, 20*250 cm, 5 μm; Mobile Phase A: CO2, Mobile Phase B: MeOH (8 mmol/L NH3.MeOH)-HPLC; Flow rate: 50 mL/min; Gradient: 25% B; 254 nm; Injection Volumn: 1 ml; number of runs: 10 to give first eluting isomer (Example 14A, RT1: 3.85, 178.3 mg). LCMS (ESI) m/z calcd for C₃₃H₄₆FN₆O₇P: 688; found: 689 (M+H). ¹H NMR (400 MHz, DMSO-d6) δ 8.27 (s, 1H), 7.86 (br s, 2H), 7.32 (d, J=7.6 Hz, 2H), 7.20-7.11 (m, 3H), 6.30-6.23 (m, 1H), 6.06-5.95 (m, 1H), 5.79 (d, J=5.6 Hz, 1H), 4.66-4.57 (m, 1H), 4.28-4.20 (m, 1H), 4.10-4.02 (m, 1H), 3.99-3.90 (m, 1H), 3.89-3.74 (m, 2H), 3.65 (s, 1H), 3.34 (s, 2H), 2.79-2.71 (m, 1H), 1.49-1.39 (m, 2H), 1.28-1.15 (m, 19H), 0.89-0.81 (m, 3H); and the second eluting isomer (Example 14B, RT: 5.13, 200 mg) as white solid which was further purified by Prep-chiral Column: CHIRALPAK IA, 2*25 cm, 5 μm; Mobile Phase A:Hex:DCM=3:1 (0.1% FA)-HPLC, Mobile Phase B: IPA-HPLC; Flow rate: 20 mL/min; Gradient: 30 B to 30 B in 8 min; 220/254 nm; RT1: 4.103; RT2:6.726; Injection Volumn: 1 ml; number of runs: 4; to give the product 99.0 mg, (RT: 4.103). LCMS (ESI) m/z calcd for C₃₃H₄₆FN₆O₇P: 688; found: 689 (M+H). ¹H NMR (400 MHz, DMSO-d6) δ 8.25 (s, 1H), 7.88 (brs, 2H), 7.35-7.26 (m, 2H), 7.18-7.09 (m, 3H), 6.32-6.24 (m, 1H), 6.05-5.94 (m, 1H), 5.82 (d, J=5.6 Hz, 1H), 4.64 (t, J=6.4 Hz, 1H), 4.26-4.17 (m, 1H), 4.14-4.06 (m, 1H), 3.94 (t, J=5.6 Hz, 2H), 3.75-3.62 (m, 2H), 2.84-2.71 (m, 1H), 1.49-1.43 (m, 2H), 1.28-1.14 (m, 21H), 0.89-0.81 (m, 3H)

### Example 15

### Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

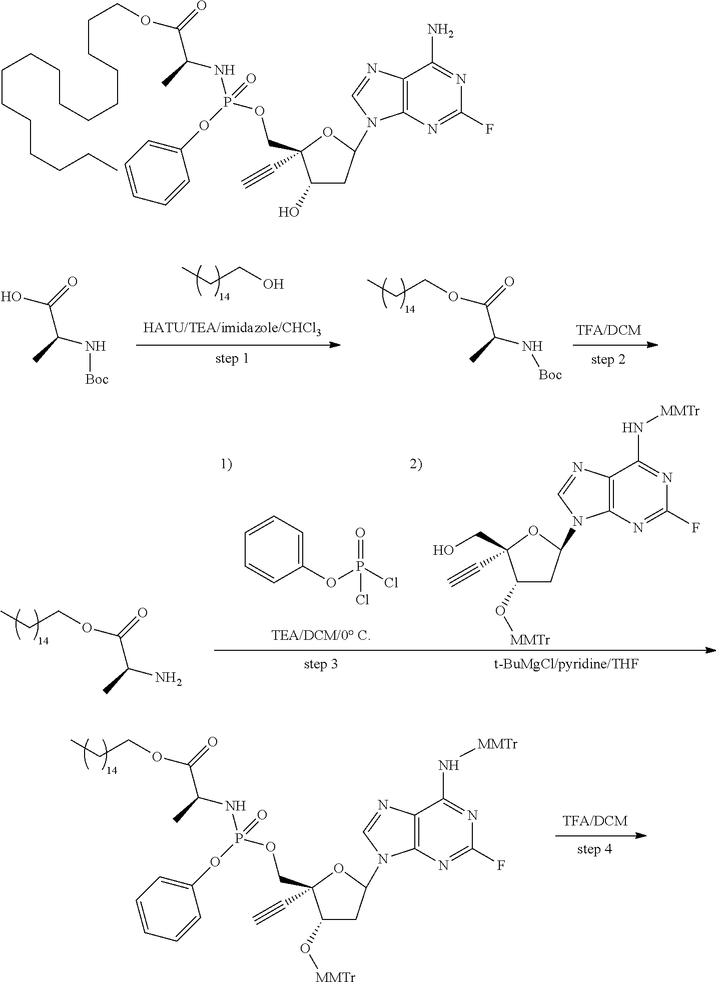

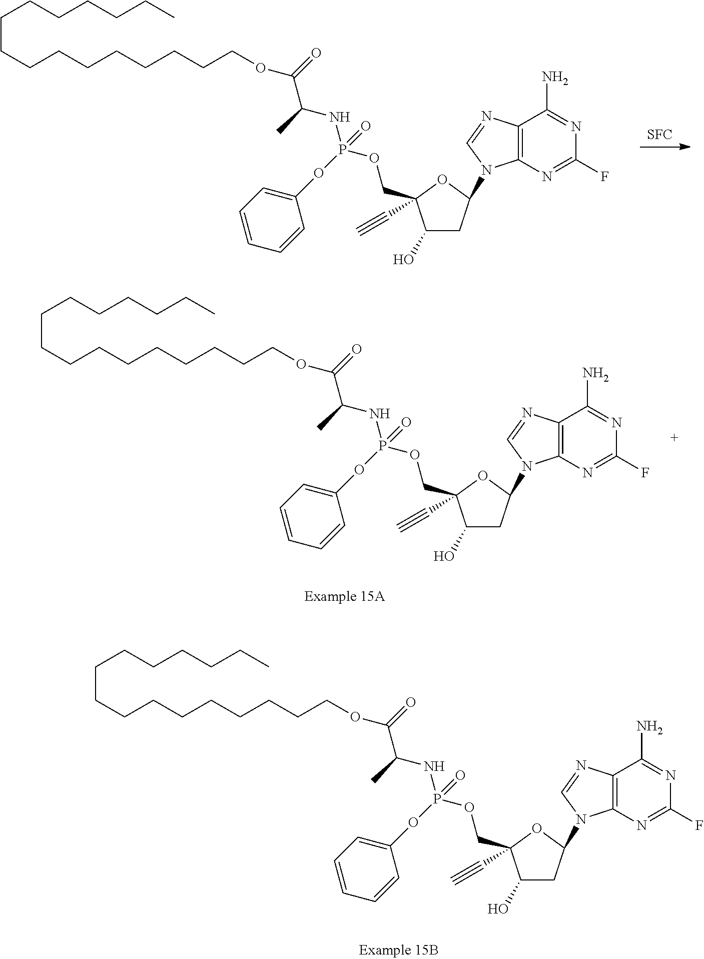

### Step 1: Hexadecyl (tert-butoxycarbonyl)-L-alaninate

To a stirred mixture of (tert-butoxycarbonyl)-L-alanine (10 g, 52.9 mmol) in chloroform (80 mL) was added HATU (30.1 g, 79 mmol), hexadecan-1-ol (19.22 g, 79 mmol), TEA (22.10 mL, 159 mmol) and imidazole (10.79 g, 159 mmol) at 0° C. The reaction mixture was stirred for 16 h at room temperature. TLC indicated completion of. reaction. The reaction mixture was quenched with water (100 mL) and extracted with DCM (100 mL*2). The organic phases were combined, washed with brine (30 mL), dried over Na₂SO₄, and concentrated under vacuum. The residue was purified by flash column chromatography (silica gel, 330 g, pet. ether:EtOAc=10:1) to give hexadecyl (tert-butoxycarbonyl)-L-alaninate (11 g, 90% purity, 49.8% yield) as a yellow oil. ¹H NMR (400 MHz, Chloroform-d): δ 5.08-4.99 (m, 1H), 4.36-4.23 (m, 1H), 4.18-4.05 (m, 2H), 1.68-1.52 (m, 5H), 1.44 (s, 9H), 1.40-1.20 (m, 26H), 0.92-0.83 (m, 3H).

### Step 2: Hexadecyl L-alaninate

To a stirred mixture of hexadecyl (tert-butoxycarbonyl)-L-alaninate (12 g, 29.0 mmol) in DCM (100 mL) was added TFA (20.0 mL, 519 mmol) at 0° C. The reaction mixture was stirred for 1 h at room temperature. LCMS indicated completion of. reaction. The reaction mixture was quenched with NaHCO₃ (100 mL). and extracted with DCM (80 mL×2). The organic phases were combined, washed with brine (15 mL), dried over Na2SO4, and concentrated under vacuum to give hexadecyl L-alaninate (7 g, 97% purity, 74.7% yield) as yellow solid. LCMS (ESI) m/z calcd for C₁₉H₃₉NO₂: 313; found: 314 (M+H). ¹H NMR (400 MHz, Chloroform-d) δ 4.20-4.09 (m, 2H), 3.95 (dd, J=7.2, 14.4 Hz, 1H), 1.68-1.59 (m, 2H), 1.55 (d, J=7.2 Hz, 3H), 1.36-1.20 (m, 26H), 0.90-0.84 (m, 3H).

### Step 3: Hexadecyl ((((2R,3S,5R)-2-ethynyl-5-(2-fluoro-6-(((4-methoxyphenyl)diphenylmethyl)amino)-9H-purin-9-yl)-3-(4-methoxyphenyl)diphenylmethoxy)tetrahydrofuran-2-yl)methoxy)(phenoxy)phosphoryl)-L-alaninate

To a solution of hexadecyl L-alaninate (2 g, 6.38 mmol) and TEA (1.937 g, 19.14 mmol) in DCM (20 mL) stirred under nitrogen at 0° C. was added. phenyl phosphorodichloridate (1.615 g, 7.65 mmol) portionwise. The reaction mixture was stirred at room temperature for 1 hour.

To a solution of. ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (2.67 g, 3.19 mmol) in pyridine (10.0 mL)/THF (10.0 mL) stirred under nitrogen at room temperature was added a solution of tert-butylmagnesium chloride/THF (8.93 mL, 8.93 mmol) dropwise. The reaction mixture was stirred at 25° C. for 0.5 hour. Then the above solution was added to this reaction mixture at room temperature. The resulting mixture was stirred at 25° C. for 16 hours. LCMS indicated completion of. reaction. The reaction mixture was evaporated in vacuum to give the crude product as red oil. The oil was purified by silica column (120 g) using 0%-60% EtOAc/pet. ether solvent gradient to give hexadecyl ((((2R,3S,5R)-2-ethynyl-5-(2-fluoro-6-(((4-methoxy-phenyl)diphenylmethyl)amino)-9H-purin-9-yl)-3-((4-methoxyphenyl)diphenylmethoxy)-tetrahydrofuran-2-yl)methoxy)(phenoxy)phosphoryl)-L-alaninate (800 mg, 80% purity, 7.78% yield) as a pale yellow semisolid. LCMS (ESI) m/z calcd for C₈₃H₉₈FN₆O₉P: 1288; found: 1289 (M+1).

### Step 4: Hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of hexadecyl ((((2R,3S,5R)-2-ethynyl-5-(2-fluoro-6-(((4-methoxyphenyl)-diphenylmethyl)amino)-9H-purin-9-yl)-3-((4-methoxyphenyl)diphenylmethoxy)tetrahydrofuran-2-yl)methoxy)(phenoxy)phosphoryl)-L-alaninate (800 mg, 0.620 mmol) in DCM (8 mL) stirred at room temperature was added TFA (0.8 mL) dropwise. The reaction mixture was stirred at room temperature for 30 min. LCMS indicated completion of. reaction. The reaction mixture was quenched with NaHCO₃ (50 mL) and extracted with DCM (30 mL×2). The organic phases were combined, washed with brine (15 mL), dried over Na2SO4, and concentrated under vacuum. The residue was purified by gel silica column (40 g, pet. ether:EtOAc=1:2) to crude product as colorless semisolid. The isomers were separated by Prep-CHIRAL-HPLC with the following conditions: Column: CHIRALPAK IA, 2*25 cm, 5 μm; Mobile Phase A: MTBE (2 mM NH3-MEOH)-HPLC, Mobile Phase B: EtOH-HPLC; Flow rate: 16 mL/min; Gradient: 30 B to 30 B in 12 min; 254/220 nm; RT1: 6.255; RT2: 10.222; Injection Volumn: 0.6 ml; number of runs: 7; to give first eluting isomer (Example 15A, RT1: 6.225, 40 mg, 92.09% purity, 7.97% yield) as white solid. LCMS (ESI) m/z calcd for C₃₇H₅₄FN₆O₇P: 744; found: 745 (M+1). ¹H NMR (400 MHz, Chloroform-d) δ 7.94 (s, 1H), 7.34-7.16 (m, 5H), 6.37-6.34 (m, 1H), 4.72 (t, J=7.6 Hz, 1H), 4.40 (d J=8.8 Hz, 2H), 4.09-4.05 (m, 3H), 3.79-3.69 (m, 1H), 2.77-2.67 (m, 3H), 1.61-1.58 (m, 2H), 1.40-1.24 (m, 29H), 0.89-0.86 (m, 3H), and second eluting isomer (Example 15B, RT2: 10.222, 27 mg, 99.80% purity, 5.83% yield) as white solid. LCMS (ESI) m/z calcd for C₃₇H₅₄FN₆O₇P: 744; found: 745 (M+1). ¹H NMR (400 MHz, Chloroform-d) δ 8.26 (s, 1H), 7.32-7.36 (m, 2H), 7.20-7.15 (m, 3H), 6.36 (t, J=4.4 Hz, 1H), 4.82 (t, J=6.8 Hz, 1H), 4.44-4.38 (m, 3H), 4.12-3.99 (m, 4H), 2.76-2.50 (m, 3H), 1.59-1.56 (m, 2H), 1.61-1.58 (m, 2H), 1.37 (d J=6.0 Hz, 2H), 1.30-1.24 (m, 26H), 0.89-0.86 (m, 3H).

### Example 16

### Docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

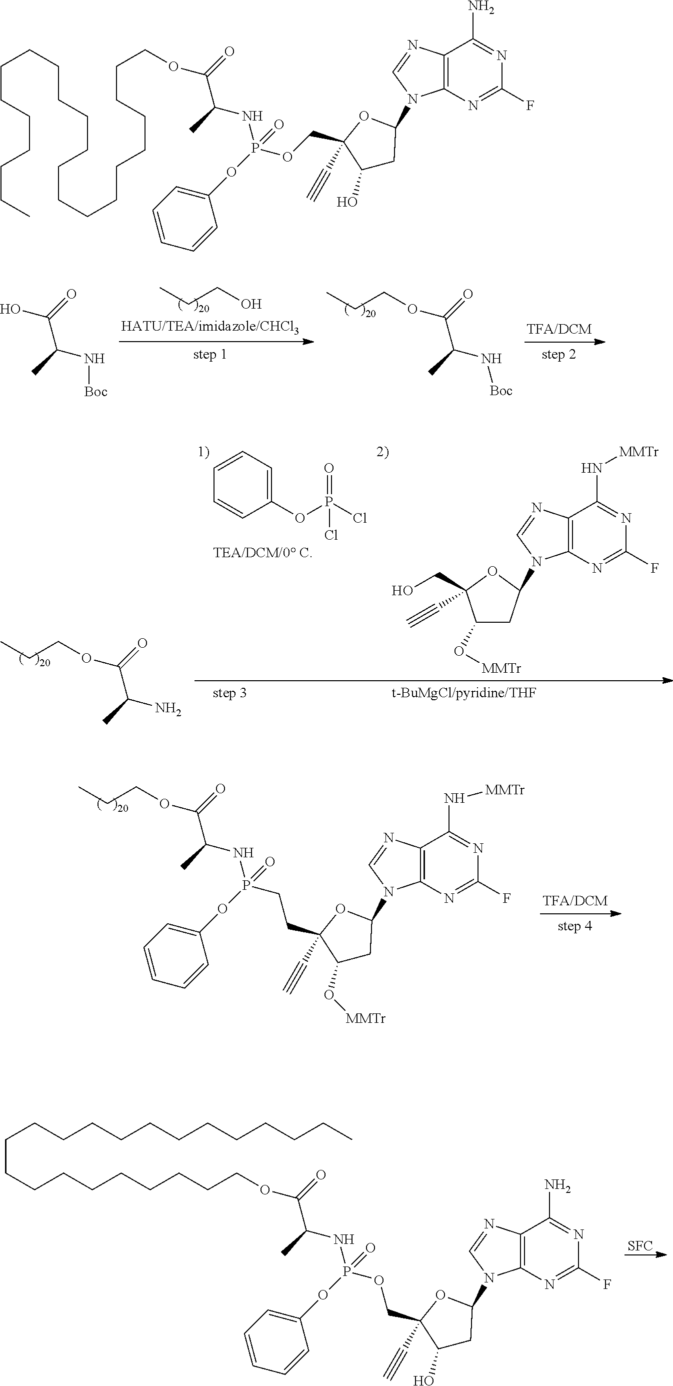

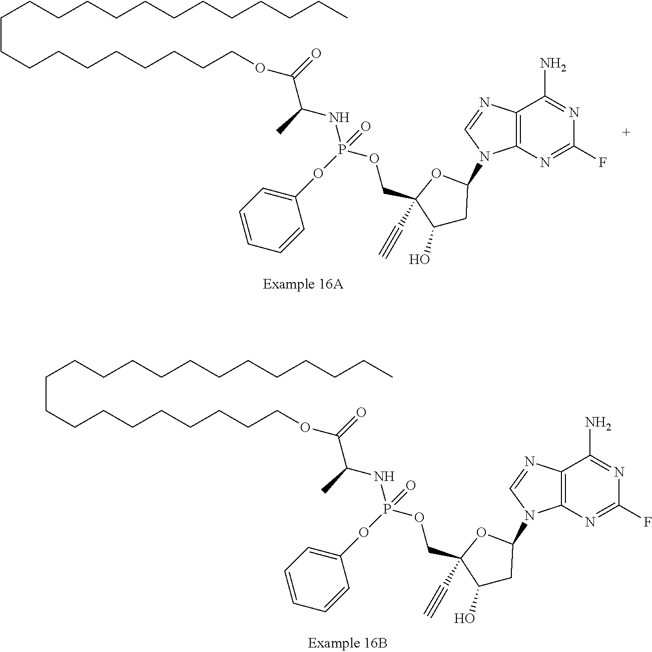 [SMILES: C#C[C@]1(COP(=O)(N[C@@H](C)C(=O)OCCCCCCCCCCCCCCCCCCCCCC)Oc2ccccc2)O[C@@H](n2cnc3c(N)nc(F)nc32)C[C@@H]1O ; C#C[C@]1(COP(=O)(N[C@@H](C)C(=O)OCCCCCCCCCCCCCCCCCCCCCC)Oc2ccccc2)O[C@@H](n2cnc3c(N)nc(F)nc32)C[C@@H]1O]

### Step 1: Docosyl (tert-butoxycarbonyl)-L-alaninate

To a stirred mixture of (tert-butoxycarbonyl)-L-alanine (10 g, 52.9 mmol) in chloroform (80 mL) was added HATU (30.1 g, 79 mmol), docosan-1-ol (19.22 g, 79 mmol), TEA (22.10 mL, 159 mmol) and imidazole (10.79 g, 159 mmol) at 0° C. The reaction mixture was stirred for 16 h at room temperature. TLC indicated completion of. reaction. The reaction mixture was quenched with water (100 mL) and extracted with DCM (100 mL*2). The organic phases were combined, washed with brine (30 mL), dried over Na₂SO₄, and concentrated under vacuum. The residue was purified by flash column chromatography (silica gel, 330 g, pet. ether:EtOAc=10:1) to give docosyl (tert-butoxycarbonyl)-L-alaninate (15 g, 100%, 68.6% yield) as yellow oil. ¹H NMR (400 MHz, Chloroform-d) δ 5.08-5.00 (m, 1H), 4.36-4.24 (m, 1H), 4.17-4.06 (m, 2H), 1.67-1.50 (m, 5H), 1.44 (br s, 9H), 1.33-1.21 (m, 38H), 0.90-0.84 (m, 3H).

### Step 2: Docosyl L-alaninate

To a stirred mixture of docosyl (tert-butoxycarbonyl)-L-alaninate (16 g, 32.1 mmol) in DCM (100 mL) was added TFA (20 mL, 519 mmol) at 0° C. The reaction mixture was stirred for 1 h at room temperature. LCMS indicated completion of. reaction. The reaction mixture was quenched with NaHCO₃ (100 mL). and extracted with DCM (80 mL×2). The organic phases were combined, washed with brine (15 mL), dried over Na2SO4 and concentrated under vacuum to give docosyl-L-alaninate (8 g, 90%, 62.0% yield) as yellow solid. LCMS (ESI) m/z calcd for C₂₅H₅₁NO₂: 397; found: 398 (M+H). ¹H NMR (400 MHz, Chloroform-d) δ 4.16-4.05 (m, 2H), 3.54 (dd, J=7.2, 14.0 Hz, 1H), 1.78 (br s, 2H), 1.67-1.56 (m, 2H), 1.41-1.15 (m, 41H), 0.93-0.82 (m, 3H).

### Step 3: Docosyl ((((2R,3S,5R)-2-ethynyl-5-(2-fluoro-6-(((4-methoxyphenyl)diphenylmethyl) amino)-9H-purin-9-yl)-3-((4-methoxyphenyl)diphenylmethoxy)tetrahydrofuran-2-yl)methoxy)(phenoxy)phosphoryl)-L-alaninate

To a solution of. docosyl L-alaninate (2 g, 5.03 mmol) and TEA (1.527 g, 17.09 mmol) in DCM (20 mL) stirred under nitrogen at 0° C. was added. phenyl phosphorodichloridate (1.273 g, 6.03 mmol) portionwise. The reaction mixture was stirred at room temperature. for 1 hour.

To a solution of. ((2R,3S,5R)-2-ethynyl-5-(2-fluoro-6-(((4-methoxyphenyl)diphenylmethyl)-amino)-9H-purin-9-yl)-3-((4-methoxyphenyl)diphenylmethoxy)tetrahydrofuran-2-yl)methanol (2.107 g, 2.51 mmol). in pyridine (10.0 mL)/THF (10.0 mL) stirred under nitrogen at room temperature was added a solution of tert-butylmagnesium chloride/THF (7.04 mL, 7.04 mmol) dropwise. The reaction mixture was stirred at 25° C. for 0.5 hour. Then, the above solution was added to this reaction mixture at room temperature. The resulting mixture was stirred at 25° C. for 16 hours. LCMS and TLC indicated completion of reaction. The reaction mixture was evaporated under vacuum to give the crude product as red oil. The oil was purified by silica column (120 g) using 0%-60% EtOAc/pet. ether solvent gradient to give docosyl ((((2R,3S,5R)-2-ethynyl-5-(2-fluoro-6-(((4-methoxyphen-yl)diphenylmethyl)amino)-9H-purin-9-yl)-3-((4-methoxyphenyl)diphenylmethoxy)tetrahydrofuran-2-yl)methoxy)(phenoxy)phosphoryl)-L-alaninate (1.8 g, 80% purity, 20.84% yield) as pale yellow semisolid. LCMS (ESI) m/z calcd for C₈₃H₉₈FN₆O₉P: 1372; found: 1373 (M+1). ¹H NMR (400 MHz, Chloroform-d) δ 7.94 (s, 1H), 7.59-7.45 (m, 4H), 7.37-7.00 (m, 26H), 6.85-6.73 (m, 4H), 6.15-6.10 (m, 1H), 4.51-3.94 (m, 4H), 3.84-3.66 (m, 10H), 2.89-2.64 (m, 2H), 2.07-1.91 (m, 2H), 1.42-1.17 (m, 41H), 0.90-0.84 (m, 3H).

### Step 4: Docosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of docosyl ((((2R,3S,5R)-2-ethynyl-5-(2-fluoro-6-(((4-methoxyphenyl)diphenylmethyl)amino)-9H-purin-9-yl)-3-((4-methoxyphenyl)diphenylmethoxy)tetrahydrofuran-2-yl)methoxy)(phenoxy)phosphoryl)-L-alaninate (2.1 g, 1.529 mmol), in DCM (15 mL) stirred at room temperature was added TFA (2 mL, 26.0 mmol) dropwise and stirred for 30 min. LCMS indicated completion of. reaction. The reaction mixture was quenched with NaHCO₃ (100 mL) and extracted with DCM (60 mL×2). The organic phases were combined, washed with brine (15 mL), dried over Na2SO4, and concentrated under vacuum. The residue was purified by silica gel column (120 g, pet. ether:EtOAc=1:1) to give the desired product as colorless semisolid. The isomers were separated by Prep-SFC with the following conditions: Column: CHIRALPAK IG, 3*25 cm, 5 μm; Mobile Phase A: CO2, Mobile Phase B: IPA; Flow rate: 80 mL/min; Gradient: 50% B; 220 nm; RT1: 6.41; RT2: 18.31; Injection Volumn: 4.5 ml; number of runs: 5. The first eluting isomer Example 16A (RT1: 6.41) was collected to give 329.7 mg (97.6% purity, 25.5% yield) as white solid. LCMS (ESI) m/z calcd for C₄₃H₆₆FN₆O₇P: 828; found: 829 (M+1). ¹H NMR (400 MHz, Chloroform-d) δ 8.03 (s, 1H), 7.36-7.10 (m, 5H), 6.63-6.01 (m, 3H), 4.71 (t, J=7.6 Hz, 1H), 4.44-4.34 (m, 2H), 4.13-3.97 (m, 3H), 3.81 (t, J=10.4 Hz, 1H), 2.77 (s, 1H), 2.76-2.62 (m, 2H), 1.63-1.54 (m, 2H), 1.39-1.17 (m, 41H), 0.90-0.84 (m, 3H); and the second isomer Example 16B (RT2: 18.31) collected to give 188.1 mg (98.06% purity, 14.5% yield) as white solid. LCMS (ESI) m/z calcd for C₄₃H₆₆FN₆O₇P: 828; found: 829 (M+1). ¹H NMR (400 MHz, Chloroform-d) δ 8.05 (s, 1H), 7.34-7.12 (m, 5H), 6.33 (dd, J=4.0, 7.2 Hz, 1H), 6.15-5.76 (m, 2H), 4.85 (t, J=6.4 Hz, 1H), 4.43-4.28 (m, 2H), 4.18-3.97 (m, 3H), 3.77 (t, J=10.4 Hz, 1H), 3.52 (br, s, 1H), 2.84-2.67 (m, 3H), 1.63-1.54 (m, 2H), 1.39 (d, J=6.8 Hz, 3H), 1.34-1.17 (m, 38H), 0.90-0.84 (m, 3H).

### Example 17

### Tridecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

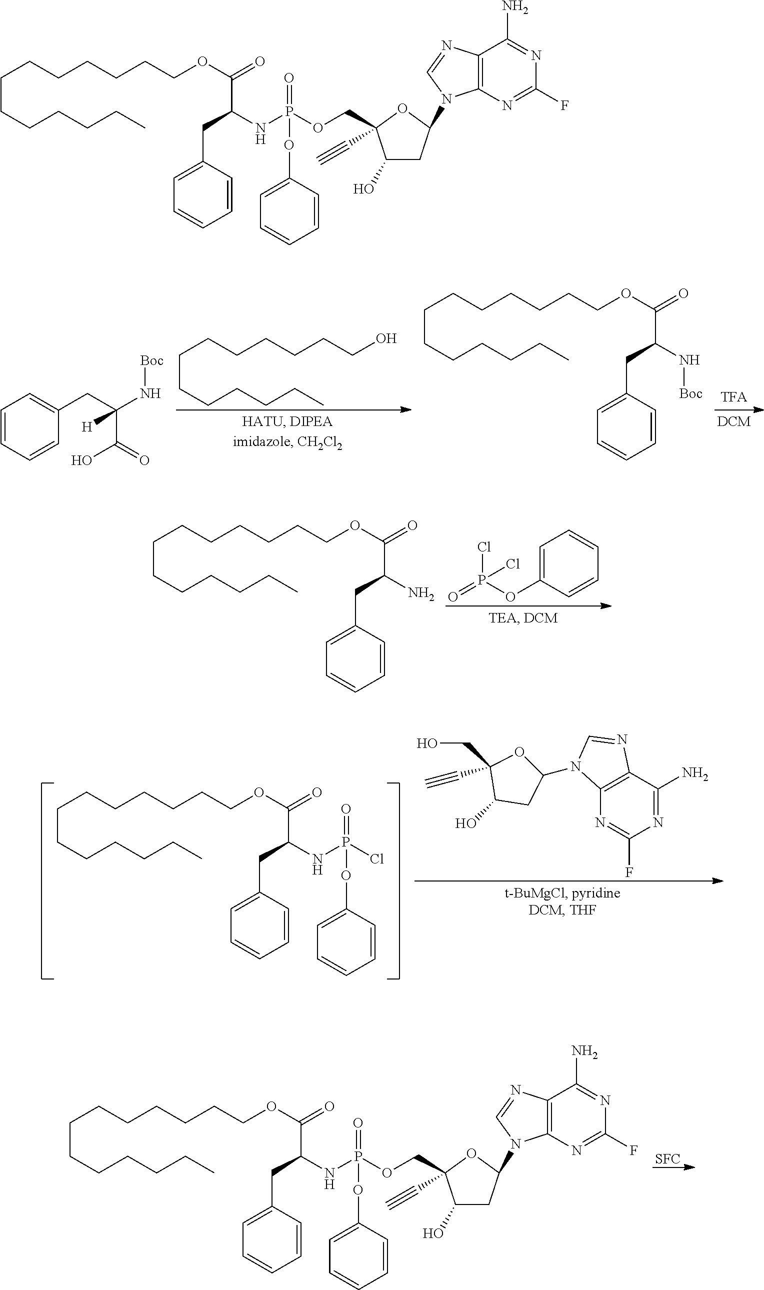

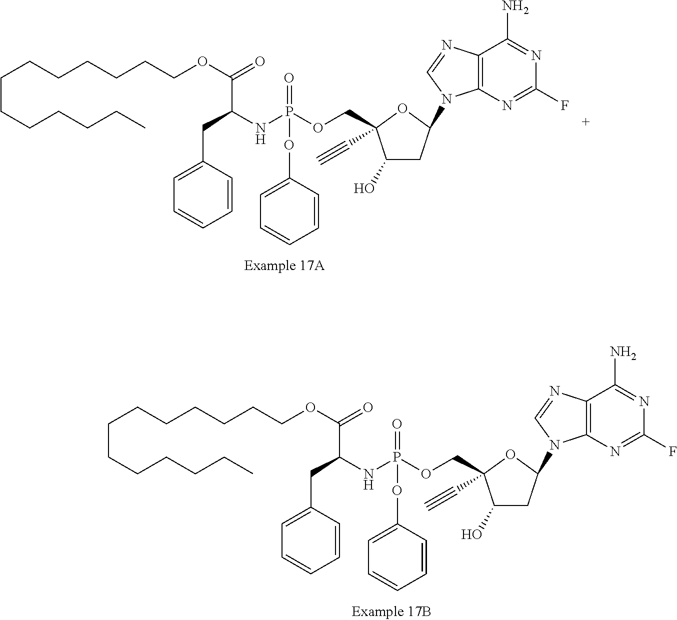 [SMILES: C#C[C@]1(COP(=O)(N[C@@H](Cc2ccccc2)C(=O)OCCCCCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OCCCCCCCCCCCCC)Oc2ccccc2)O[C@@H](n2cnc3c(N)nc(F)nc32)C[C@@H]1O]

### Step 1: Tridecyl (tert-butoxycarbonyl)-L-phenylalaninate

To a solution of tridecan-1-ol (10.80 g, 53.9 mmol), (tert-butoxycarbonyl)-L-phenylalanine (13 g, 49.0 mmol) and 2-(3H-[1,2,3]triazolo[4,5-b]pyridin-3-yl)-1,1,3,3-tetramethylisouronium hexafluorophosphate(V) (27.9 g, 73.5 mmol) in DCM (100 mL) was added 1H-imidazole (10.01 g, 147 mmol) and N-ethyl-N-isopropylpropan-2-amine (19.00 g, 147 mmol). The resulting mixture was stirred at 25° C. overnight. LCMS showed completion of reaction. The reaction was diluted with water (200 mL) and DCM (100 mL). The organic layer was separated and the aqueous layer was extracted with DCM (2×50 mL). The combined organic layers were washed with brine (200 mL), dried with Na₂SO₄, filtered and concentrated to give crude product. The residue was purified by combiflash (silica gel column 330 g, hexane:ethyl acetate=20:1). The appropriate fractions containing desired product were combined and concentrated in vacuo to give tridecyl (tert-butoxycarbonyl)-L-phenylalaninate (19.2 g, 34.6 mmol, 70.6% yield) as yellow solid. LCMS (M+Na)=447.3; Retention time (0.1% TFA)=3.25 min.

### Step 2: Tridecyl L-phenylalaninate

To a solution of tridecyl (tert-butoxycarbonyl)-L-phenylalaninate (19 g, 42.4 mmol) in DCM (100 mL) was added 2,2,2-trifluoroacetic acid (35 g, 307 mmol). The resulting mixture was stirred at 25° C. overnight. LCMS showed completion of reaction. The reaction was quenched with water (200 mL) and DCM (100 mL) then the organic layer was separated. The aqueous layer was extracted with DCM (2×50 mL). The combined organic layers were washed with brine (200 mL), dried with Na₂SO₄, filtered and concentrated to give tridecyl L-phenylalaninate (15.2 g, 33.1 mmol, 78% yield) as a yellow gum which was used in the next step without purification. LCMS (M+H)=348.0; Retention time (0.1% TFA)=4.65 min.

### Step 3: Tridecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of tridecyl L-phenylalaninate (1,481 mg, 4.26 mmol), triethylamine (0.594 mL, 4.26 mmol) in DCM (30 mL) was added phenyl phosphorodichloridate (899 mg, 4.26 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (500 mg, 1.705 mmol) in pyridine (10 mL)/THF (20 mL) was added tert-butylmagnesium chloride (4.26 mL, 4.26 mmol) dropwise under an atmosphere of nitrogen, then the mixture was stirred at room temperature for 30 min. The first prepared mixture was added dropwise to the second prepared mixture at room temperature. The resulting mixture was stirred at room temperature for 2 h. LCMS indicated completion of reaction. The reaction mixture was quenched with water, partitioned between dichloromethane (100 mL) and water 100 (mL). The organic phase was washed with water (100 mL) and saturated brine (100 mL), dried over sodium sulphate and evaporated in vacuo to give the crude product. The residue was purified by silica gel chromatography (80 g, MeOH: DCM=15:1) to give tridecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (590 mg, 0.722 mmol, 42.3% yield). LCMS (M+H)=779.3; Retention time (0.1% TFA)=2.22 min. The diastereomers were separated by Prep-SFC (Instrument: SFC-80 (Thar, Waters); Column: OZ 20×250 mm, 10 μm (Deicel): Column temperature: 40° C.; Mobile phase: CO2/MeOH (0.2% ammonia/methanol)=50/50; Flow rate: 80 g/min; Back pressure: 100 bar; Detection wavelength: 214 nm; Cycle time: 8 min; Sample solution: 590 mg dissolved in 50 ml methanol; Injection volume: 3 mL) to give first eluting isomer (Example 17A, RT1: 1.66 min, 100.5 mg, 0.127 mmol, 98.59%, yield: 16.79%) as an off-white solid; LCMS (M+H)=779.0; Retention time (0.1% TFA)=2.62 min. 1H NMR (400 MHz, DMSO) δ 8.20 (s, 1H), 7.85 (s, 2H), 7.25-7.17 (m, 5H), 7.13-7.07 (m, 3H), 6.98 (d, J=8.5 Hz, 2H), 6.27 (dd, J=7.4, 5.2 Hz, 1H), 6.13 (dd, J=12.9, 10.4 Hz, 1H), 5.78 (d, J=5.3 Hz, 1H), 4.54 (dd, J=11.5, 6.6 Hz, 1H), 4.05-3.96 (m, 2H), 3.91-3.83 (m, 3H), 3.65 (s, 1H), 2.93-2.86 (m, 1H), 2.80 (dd, J=13.5, 8.0 Hz, 1H), 2.73-2.66 (m, 1H), 2.46-2.40 (m, 1H), 1.40-1.32 (m, 2H), 1.18 (d, J=35.8 Hz, 20H), 0.85 (t, J=6.8 Hz, 3H). and second eluting isomer (Example 17B, RT2: 2.77 min, 85.5 mg, 0.107 mmol, 97.28%, yield: 14.10%) as an off-white solid; LCMS (M+H)=779.0; Retention time (0.1% TFA)=2.22 min. 1H NMR (400 MHz, DMSO) δ 8.24 (s, 1H), 7.85 (brs, 2H), 7.31-7.20 (m, 6H), 7.16-7.14 (m, 3H), 7.02 (d, J=8.5 Hz, 2H), 6.23 (dd, J=7.6, 4.9 Hz, 1H), 6.09 (dd, J=12.6, 10.5 Hz, 1H), 5.74 (s, 1H), 4.52 (t, J=7.2 Hz, 1H), 4.04-3.88 (m, 2H), 3.87-3.75 (m, 3H), 3.62 (s, 1H), 2.95-2.88 (m, 1H), 2.81-2.66 (m, 2H), 2.46-2.40 (m, 1H), 1.35-1.31 (m, 2H), 1.22 (s, 14H), 1.16-1.10 (m, 6H), 0.85 (t, J=6.8 Hz, 3H).

### Example 18

### Tetradecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

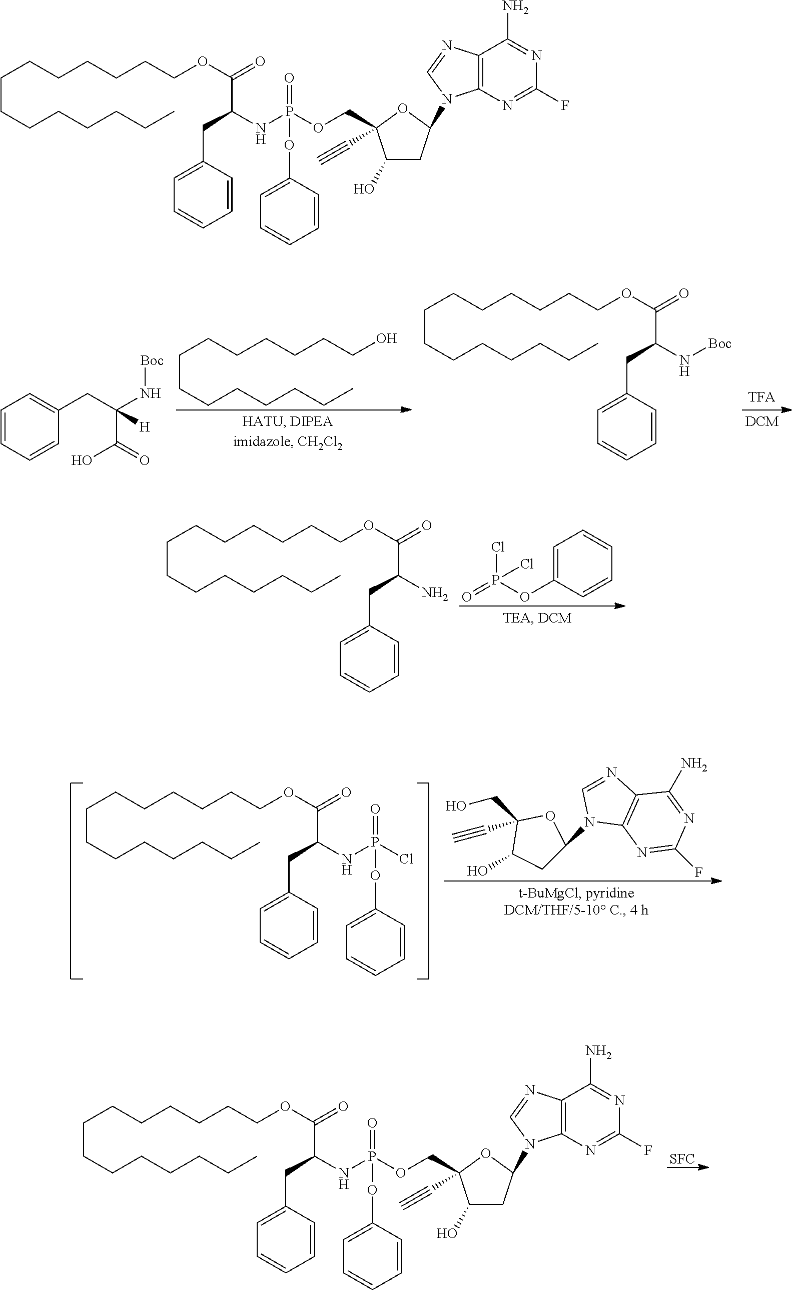

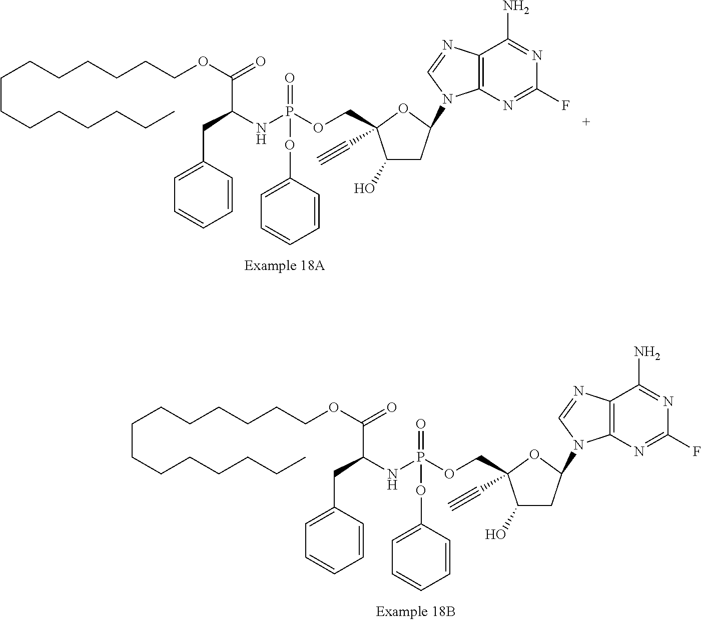 [SMILES: C#C[C@]1(COP(=O)(N[C@@H](Cc2ccccc2)C(=O)OCCCCCCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OCCCCCCCCCCCCCC)Oc2ccccc2)O[C@@H](n2cnc3c(N)nc(F)nc32)C[C@@H]1O]

### Step 1: Tetradecyl (tert-butoxycarbonyl)-L-phenylalaninate

To a solution of (tert-butoxycarbonyl)-L-phenylalanine (15 g, 56.5 mmol), 2-(3H-[1,2,3]triazolo[4,5-b]pyridin-3-yl)-1,1,3,3-tetramethylisouronium hexafluorophosphate(V) (32.2 g, 85 mmol) and tetradecan-1-ol (14.55 g, 67.8 mmol) in DCM (200 mL) was added 1H-imidazole (11.55 g, 170 mmol) and N-ethyl-N-isopropylpropan-2-amine (21.92 g, 170 mmol). The resulting mixture was stirred at 25° C. overnight. TLC showed completion of the reaction. The reaction was quenched with water (200 mL) and DCM (100 mL). Then, the organic layer was separated and the aqueous layer was extracted with DCM (2×200 mL). The combined organic layers were washed with brine (200 mL), dried over Na₂SO₄, filtered and concentrated to give crude product. The residue was purified by combiflash (silica gel column 330 g, hexane:ethyl acetate=20:1). The appropriate fractions containing desired product were combined and solvent was removed in vacuum to give tetradecyl (tert-butoxycarbonyl)-L-phenylalaninate (20 g, 41.2 mmol, 72.8% yield) as yellow solid. LCMS (M+Na)=485.0; Retention time (0.1% TFA)=3.56 min.

### Step 2: Tetradecyl L-phenylalaninate

To a solution of tetradecyl (tert-butoxycarbonyl)-L-phenylalaninate (30 g, 65.0 mmol) in DCM (150 mL) was added 2,2,2-trifluoroacetic acid (37.0 g, 325 mmol). The resulting mixture was stirred at room temperature overnight. LCMS showed completion of reaction. The reaction mixture was concentrated in vacuum to remove the volatiles and the pH of the mixture was adjusted to ˜5 with 1 N NaHCO₃. The resulting cloudy yellow solution was then partitioned with DCM, and the layers were separated. The aqueous layer was extracted with DCM, and the combined organic layers were dried over MgSO₄, filtered and concentrated in vacuum to give crude tetradecyl L-phenylalaninate (16 g, 42.0 mmol, 64.7% yield) which was used in the next step without purification. LCMS (M+H)=362.3; Retention time (0.1% TFA)=2.096 min.

### Step 3: Tetradecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of tetradecyl L-phenylalaninate (2.96 g, 8.18 mmol), triethylamine (1.141 mL, 8.18 mmol) in DCM (60 mL) was added phenyl phosphorodichloridate (1.223 mL, 8.18 mmol) in DCM (1 mL) dropwise under an atmosphere of nitrogen at about 5° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (1.2 g, 4.09 mmol) in THF (20 mL)/pyridine (10 mL) was added tert-butylmagnesium chloride (10.23 mL, 10.23 mmol) dropwise under an atmosphere of nitrogen, then the mixture was stirred at room temperature for 30 min. The first prepared mixture was added dropwise to the second prepared mixture at room temperature. The resulting mixture was stirred at room temperature for 2 h. LCMS indicated completion of reaction. The mixture was filtered and concentrated to afford yellow solid. The solid was dissolved in EtOAc (40 mL) and partitioned with HCl (0.05 N, 20 mL). The layers were separated, and the aqueous layer was extracted with EtOAc (40 mL). The combined organic phases were washed with brine, dried over Na₂SO₄, filtered and concentrated to afford the crude product. The residue was purified by reverse phase chromatography (SepaFlash® C18, 0-100% CH₃CN/10 mM NH₄—HCO₃H₂O, 100%) to give tetradecyl ((((2R,3S,5R)-5-(6-amino fluoro-9H-purin-9-yl)-2-ethynyl-3-hydroxytetrahydrofuran yl)methoxy)(phenoxy)phosphoryl)-L-phenylalaninate (430 mg, 0.500 mmol, 12.22% yield). LCMS (M+H)=793.2; Retention time (0.1% TFA)=2.31 min. The diastereomers were separated by Prep-SFC (Instrument: SFC-80 (Thar, Waters), Column: IG 20×250 mm, 10 μm (Daicel), Column temperature: 40° C., Mobile phase: CO₂/EtOH (1.0% ammonia/methanol)=40/60, Flow rate: 80 g/min, Back pressure: 100 bar, Detection wavelength: 214 nm, Cycle time: 13.4 min)) to give first elution isomer (Example 18A, RT1: 3.59 min, 200 mg, 0.252 mmol, 28.6% yield); LCMS (M+H)=793.2; Retention time (0.1% TFA)=2.298 min, HPLC: Retention time (0.1% NH₄HCO₃)=12.709 min; and second elution isomer (Example 18B, RT2: 5.18 min, 210 mg, 0.250 mmol, 29.7% yield) as gray solid. LCMS: no mass, Retention time (0.1% TFA)=2.306 min, HPLC: Retention time (0.1% NH₄HCO₃)=12.729 min. Each isomer was repurified by SFC to provide pure material. First elution isomer (Example 18A, RT1: 3.59 min, 136 mg, 0.172 mmol); LCMS (M+H)=793.3; Retention time (0.1% TFA)=2. 305 min, HPLC: Retention time (0.1% TFA)=13.715 min. ¹H NMR (400 MHz, CDCl₃) δ 7.76 (s, 1H), 7.31-7.27 (m, 2H), 7.25 (s, 1H), 7.22 (d, J=5.6 Hz, 2H), 7.15 (dd, J=7.6, 6.2 Hz, 3H), 7.06 (dd, J=7.5, 1.7 Hz, 2H), 6.32 (dd, J=7.4, 4.3 Hz, 1H), 6.09 (s, 2H), 4.67-4.59 (m, 1H), 4.24-4.14 (m, 2H), 4.03 (td, J=6.7, 1.3 Hz, 2H), 3.97 (dd, J=11.6, 8.7 Hz, 1H), 3.69-3.61 (m, 2H), 3.03-2.91 (m, 2H), 2.70 (s, 1H), 2.64 (dt, J=12.0, 5.2 Hz, 1H), 2.60-2.53 (m, 1H), 1.78 (s, 2H), 1.25 (d, J=3.5 Hz, 22H), 0.88 (t, J=6.8 Hz, 3H) and second elution isomer (Example 18B, RT2: 5.18 min, 205 mg, 0.259 mmol) was obtained as brick solid. LCMS: no mass, Retention time (0.1% TFA)=2.303 min, HPLC: Retention time (0.1% TFA)=13.76 min. ¹H NMR (400 MHz, CDCl₃) δ 7.90 (s, 1H), 7.23 (d, J=5.3 Hz, 2H), 7.20 (d, J=7.2 Hz, 2H), 7.15 (d, J=11.9 Hz, 1H), 7.10 (dd, J=13.2, 4.9 Hz, 5H), 6.31 (dd, J=7.2, 4.4 Hz, 1H), 6.21 (s, 2H), 4.72 (dd, J=12.1, 7.4 Hz, 1H), 4.26 (dt, J=11.5, 3.6 Hz, 2H), 4.20 (dd, J=11.2, 8.6 Hz, 1H), 4.02 (t, J=6.7 Hz, 2H), 3.96 (d, J=11.0 Hz, 1H), 3.75 (d, J=4.9 Hz, 1H), 2.99 (d, J=6.2 Hz, 2H), 2.74-2.70 (m, 1H), 2.70 (s, 1H), 2.66 (dd, J=13.7, 7.8 Hz, 1H), 1.82 (s, 2H), 1.23 (d, J=10.1 Hz, 22H), 0.87 (t, J=6.8 Hz, 3H).

### Example 19

### Pentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

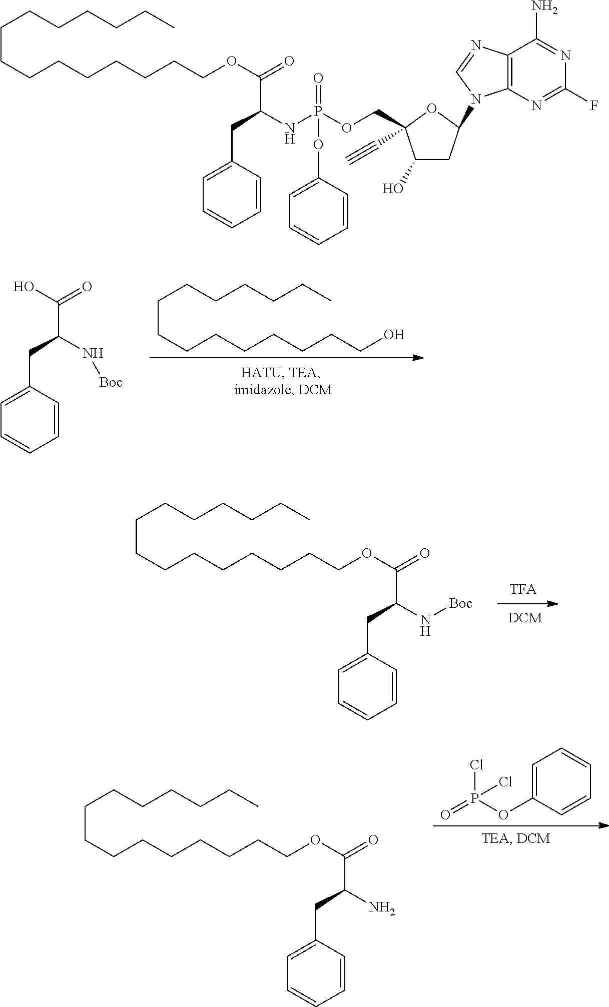

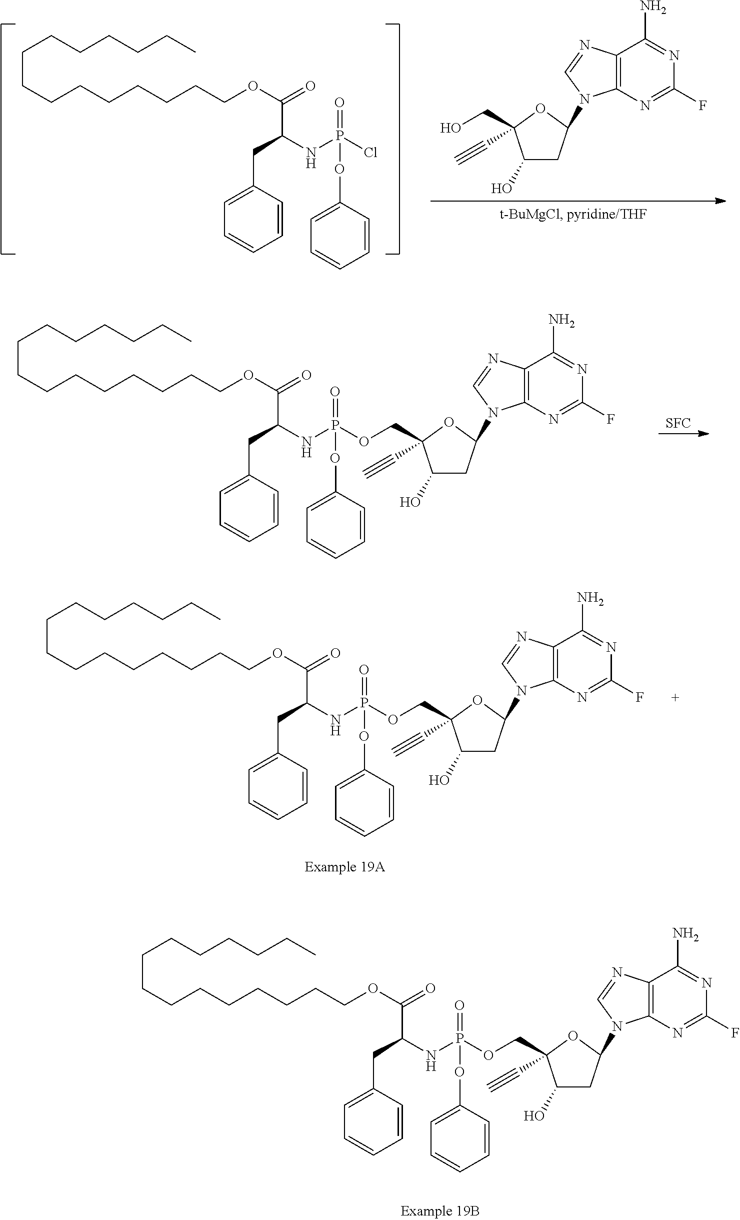

### Step 1: Pentadecyl (tert-butoxycarbonyl)-L-phenylalaninate

To a solution of pentadecan-1-ol (12.54 g, 54.9 mmol), (tert-butoxycarbonyl)-L-phenylalanine (11.2 g, 42.2 mmol) and 2-(3H-[1,2,3]triazolo[4,5-b]pyridin-3-yl)-1,1,3,3-tetramethylisouronium hexafluorophosphate(V) (24.08 g, 63.3 mmol) in DCM (100 mL) was added 1H-imidazole (8.62 g, 127 mmol) and N-ethyl-N-isopropylpropan-2-amine (16.37 g, 127 mmol). The resulting mixture was stirred at 25° C. overnight. LCMS showed the reaction was completed. The reaction was diluted with water (200 mL) and DCM (100 mL) then the organic layer was separated. The aqueous layer was extracted by DCM (50 mL×2), the combined organic layers were washed by brine (200 mL), dried over Na2SO4, concentrated to give crude product. The residue was purified by combiflash (silica gel column 330 g, hexane:ethyl acetate=20:1). The appropriate fractions were combined and solvent removed in vacuo to give pentadecyl (tert-butoxycarbonyl)-L-phenylalaninate (19.5 g, 36.2 mmol, 86% yield) was obtained as yellow solid. LCMS (M+Na)=498.1 and (M−99)=376.3; retention time: (0.1% TFA)=3.929.

### Step 2: Pentadecyl L-phenylalaninate

To a solution of pentadecyl (tert-butoxycarbonyl)-L-phenylalaninate (19.2 g, 40.4 mmol) in DCM (100 mL) was added 2,2,2-trifluoroacetic acid (35 g, 307 mmol). The resulting mixture was stirred at 25° C. overnight. LCMS showed completion of reaction. The reaction mixture was quenched with saturated sodium bicarbonate solution (till pH=7) then the organic layer was separated. The aqueous layer was extracted with DCM (2×50 mL). The combined organic layers were washed with brine (200 mL), dried with Na₂SO₄, filtered and concentrated to give crude pentadecyl L-phenylalaninate (15.1 g, 26.9 mmol, 66.6% yield) as a yellow solid. LCMS (M+H)=376.3; Retention time (0.1% TFA)=2.055 min.

### Step 3: Pentadecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

To a cold (ice-water bath) solution of pentadecyl L-phenylalaninate (3202 mg, 8.52 mmol), triethylamine (1.188 mL, 8.52 mmol) in dichloromethane (60 mL) was added phenyl phosphorodichloridate (1799 mg, 8.52 mmol) in DCM (10 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1000 mg, 3.41 mmol) in THF (40 mL)/pyridine (20 mL) was added tert-butylmagnesium chloride (8.52 mL, 8.52 mmol) dropwise under an atmosphere of nitrogen, then the mixture was stirred at room temperature for 30 min. The first prepared mixture was added dropwise to the second prepared mixture at room temperature. The resulting mixture was stirred room temperature for 16 h. LCMS indicated completion of reaction. The reaction mixture was filtered and concentrated, the mixture was added EtOAc (120 mL), and washed with water (80 mL) twice. The organic phase was dried with Na₂SO₄, filtererd, concentrated and purified by Prep-HPLC (Column: 100 g, 0-100% CH3CN/10 mM NH4HCO3 H2O) to give pentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (460 mg, 0.532 mmol, 15.60% yield) as a white solid. LCMS (M+H)=807.3; Retention time (10 mM NH₄HCO₃)=3.175 min. The diastereomers were separated by Prep-SFC (Instrument: SFC-80 (Thar, Waters); Column: OZ 20×250 mm, 10 μm (Deicel): Column temperature: 40° C.; Mobile phase: CO₂/MeOH (0.2% ammonia/methanol)=75/25; Flow rate: 100 g/min; Back pressure: 113 bar; Detection wavelength: 214 nm; Cycle time: 8 min; Sample solution: 500 mg dissolved in 50 ml methanol; Injection volume: 3 mL) to give first eluting isomer (Example 19A, RT1: 2.77 min, 117.5 mg, 0.146 mmol, 23.50% yield) as an off-white solid; LCMS (M+H)=807.0; Retention time (10 mM NH₄HCO₃)=3.153 min. ¹H NMR (400 MHz, CDCl₃) δ 7.76 (s, 1H), 7.35-7.27 (m, 3H), 7.23 (d, J=6.0 Hz, 2H), 7.19-7.13 (m, 3H), 7.10-7.03 (m, 2H), 6.32 (dd, J=7.3, 4.3 Hz, 1H), 5.90 (s, 2H), 4.62 (t, J=7.5 Hz, 1H), 4.28-4.11 (m, 2H), 4.10-3.99 (m, 2H), 3.95 (dd, J=11.7, 9.0 Hz, 1H), 3.55 (t, J=11.0 Hz, 2H), 2.98 (qd, J=14.1, 6.4 Hz, 2H), 2.70 (d, J=5.4 Hz, 1H), 2.68-2.53 (m, 2H), 1.69 (s, 2H), 1.25 (s, 24H), 0.88 (t, J=6.8 Hz, 3H). and second eluting isomer (Example 19B, RT2: 4.9 min, 203.1 mg, 0.252 mmol, 99.83, 40.6% yield) as an off-white solid. LCMS (M+H)=807.0; Retention time (10 mM NH₄HCO₃)=2.794 min. ¹H NMR (400 MHz, CDCl₃) δ 7.90 (s, 1H), 7.28 (t, J=3.8 Hz, 1H), 7.25-7.07 (m, 10H), 6.31 (dd, J=7.2, 4.3 Hz, 1H), 6.13 (s, 2H), 4.72 (dd, J=12.5, 7.5 Hz, 1H), 4.29-4.18 (m, 3H), 4.02 (t, J=6.7 Hz, 2H), 3.93 (d, J=10.6 Hz, 1H), 3.68 (s, 1H), 2.99 (d, J=6.2 Hz, 2H), 2.75-2.65 (m, 3H), 1.79 (s, 2H), 1.24 (d, J=9.4 Hz, 24H), 0.88 (t, J=6.8 Hz, 3H).

### Example 20

### Heptadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

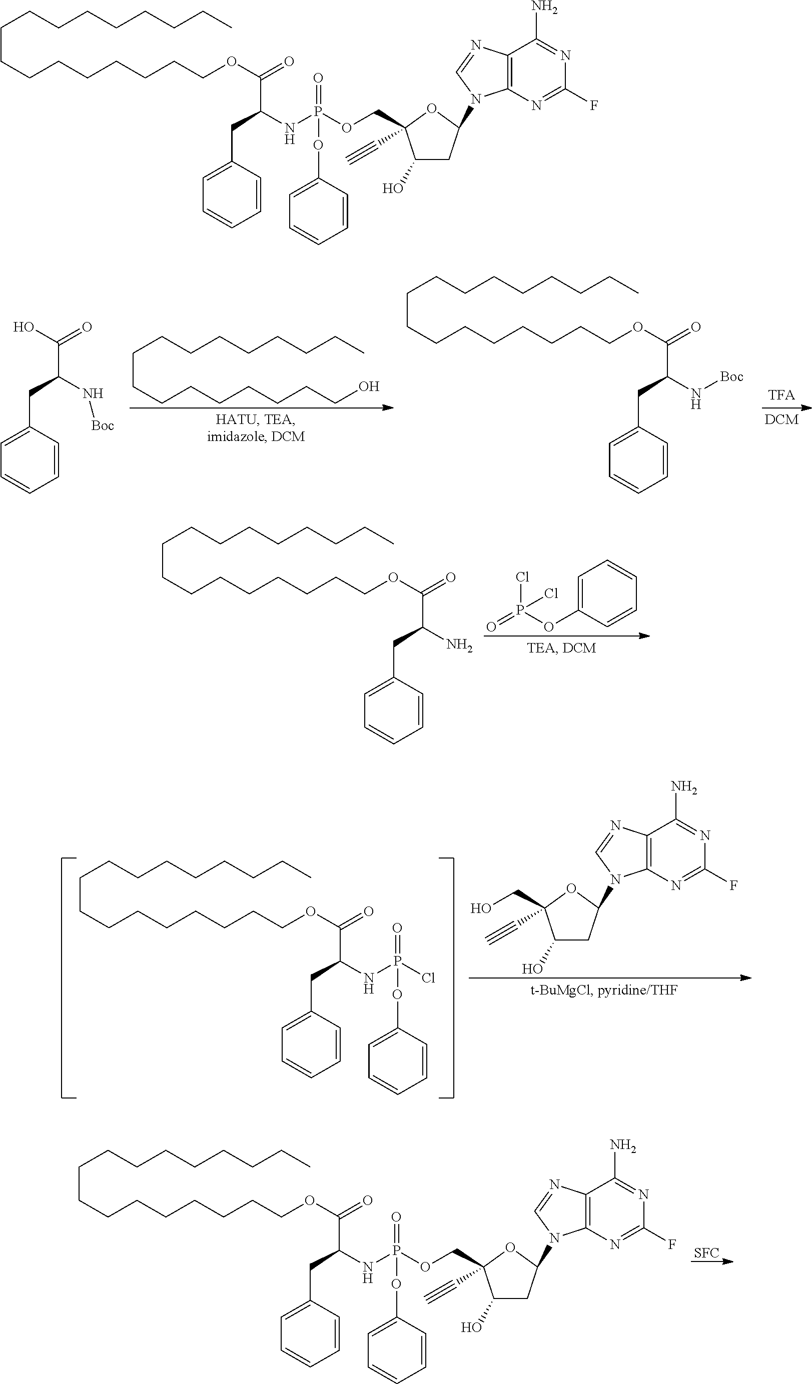

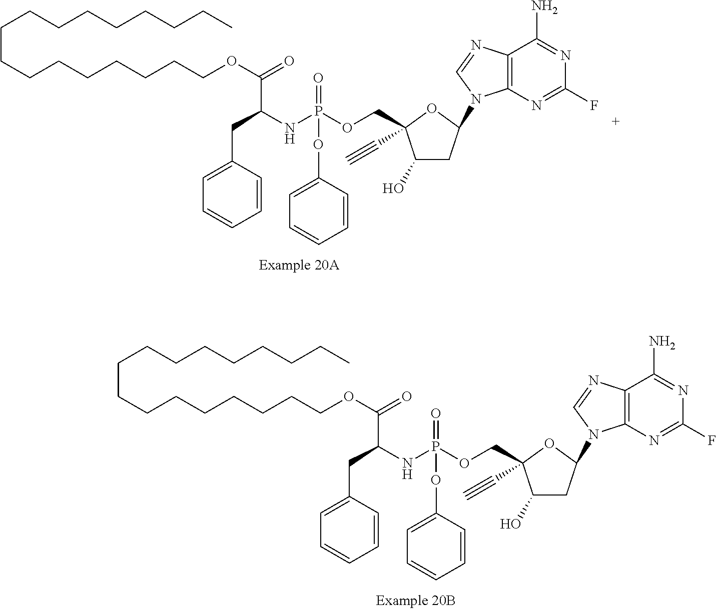 [SMILES: C#C[C@]1(COP(=O)(N[C@@H](Cc2ccccc2)C(=O)OCCCCCCCCCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OCCCCCCCCCCCCCCCCC)Oc2ccccc2)O[C@@H](n2cnc3c(N)nc(F)nc32)C[C@@H]1O]

### Step 1: Heptadecyl (tert-butoxycarbonyl)-L-phenylalaninate

To a solution of (tert-butoxycarbonyl)-L-phenylalanine (10.0 g, 37.7 mmol), 2-(3H-[1,2,3]triazolo[4,5-b]pyridin-3-yl)-1,1,3,3-tetramethylisouronium hexafluorophosphate(V) (21.50 g, 56.5 mmol), and heptadecan-1-ol (11.60 g, 45.2 mmol) in DCM (100 mL) was added 1H-imidazole (7.70 g, 113 mmol) and N-ethyl-N-isopropylpropan-2-amine (14.61 g, 113 mmol). The resulting mixture was stirred at 25° C. overnight. TLC showed completion of reaction. The reaction was quenched with water (200 mL) and then the organic layer was separated. The aqueous layer was extracted with DCM (2×50 mL). The combined organic layers were washed with brine (200 mL), dried with Na₂SO₄, filtered and concentrated to give crude product. The crude product was purified by flash chromatography (silica gel column 330 g, hexane:ethyl acetate=20:1) to give heptadecyl (tert-butoxycarbonyl)-L-phenylalaninate (18.5 g, 36.7 mmol, 97% yield) as a yellow solid. LCMS: no mass, ¹H NMR (400 MHz, CDCl₃) δ 7.31-7.27 (m, 2H), 7.23 (d, J=7.3 Hz, 1H), 7.13 (d, J=7.1 Hz, 2H), 4.98 (d, J=8.2 Hz, 1H), 4.57 (dd, J=14.1, 6.1 Hz, 1H), 4.07 (t, J=6.6 Hz, 2H), 3.12-3.02 (m, 2H), 1.59-1.54 (m, 2H), 1.41 (s, 9H), 1.25 (s, 30H), 0.87 (t, J=6.7 Hz, 3H).

### Step 2: Heptadecyl L-phenylalaninate

To a solution of heptadecyl (tert-butoxycarbonyl)-L-phenylalaninate (19.7 g, 39.1 mmol) in DCM (150 mL) was added TFA (20.27 mL, 263 mmol). The resulting mixture was stirred at room temperature overnight. LCMS showed completion of reaction. The reaction was quenched with saturated sodium bicarbonate solution (till pH=7) then the organic layer was separated. The aqueous layer was extracted with DCM (2×50 mL). The combined organic layers were washed with brine (200 mL), dried with Na₂SO₄, filtered and concentrated to give crude heptadecyl L-phenylalaninate (15.3 g, 19.31 mmol, 49.4% yield) as a yellow solid which was used in the next step without purification. LCMS (M+H)=404.3; Retention time (0.1% TFA)=2.175 min.

### Step 3: Heptadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of heptadecyl L-phenylalaninate (2753 mg, 6.82 mmol), triethylamine (1.188 mL, 8.52 mmol) in dichloromethane (60 mL) was added phenyl phosphorodichloridate (1799 mg, 8.52 mmol) in DCM (10 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1000 mg, 3.41 mmol) in THF (40 mL)/pyridine (20 mL) was added tert-butylmagnesium chloride (8.52 mL, 8.52 mmol) dropwise under an atmosphere of nitrogen, then the mixture was stirred at room temperature for 30 min. The first prepared mixture was added dropwise to the second prepared mixture at room temperature. The resulting mixture was stirred room temperature for 2 h. LCMS indicated completion of reaction. The reaction mixture was filtered, concentrated and the residue was diluted with EtOAc (120 mL), and washed with water (80 mL) twice. The organic phase was dried with Na₂SO₄, filtered, concentrated and purified by Prep-HPLC (Column: 100 g, 0-100% CH3CN/10 mM NH4HCO3 H2O) to give heptadecyl ((((2R,3S,5R)-5-(6-amino-2-fluoro-9H-purin-9-yl)-2-ethynyl-3-hydroxytetrahydrofuran yl)methoxy)(phenoxy)phosphoryl)-L-phenylalaninate (500 mg, 0.583 mmol, 17.10% yield) as a white solid. LCMS (M+H)=835.5; Retention time (10 mM NH₄HCO₃)=3.546 min. The diastereomers were separated by Prep-SFC (Instrument: SFC-80 (Thar, Waters); Column: OZ 20×250 mm, 10 μm (Deicel): Column temperature: 40° C.; Mobile phase: CO₂/MeOH (0.2% ammonia/methanol)=75/25; Flow rate: 100 g/min; Back pressure: 113 bar; Detection wavelength: 214 nm; Cycle time: 8 min; Sample solution: 500 mg dissolved in 50 ml methanol; Injection volume: 3 mL) to give first eluting isomer (Example 20A, RT1: 3.02 min, 145.8 mg, 0.168 mmol, 25.5% yield) as an off-white solid; LCMS (M+H)=835.0; Retention time (10 mM NH₄HCO₃)=3.552 min. ¹H NMR (400 MHz, CDCl₃) δ 7.77 (s, 1H), 7.30 (d, J=8.0 Hz, 2H), 7.23 (s, 3H), 7.16 (d, J=7.3 Hz, 3H), 7.07 (d, J=7.1 Hz, 2H), 6.32 (dd, J=7.3, 4.2 Hz, 1H), 6.09 (s, 2H), 4.63 (d, J=7.3 Hz, 1H), 4.22-4.15 (m, 2H), 4.03 (t, J=6.6 Hz, 2H), 3.99-3.94 (m, 1H), 3.71-3.63 (m, 2H), 2.97 (dd, J=14.3, 7.2 Hz, 2H), 2.71 (s, 1H), 2.61 (ddd, J=13.6, 13.1, 7.0 Hz, 2H), 1.77 (s, 2H), 1.25 (s, 28H), 0.87 (d, J=7.0 Hz, 3H). and second eluting isomer (Example 20B, RT2: 5.35 min, 240.5 mg, 0.281 mmol, 42.6% yield) as an off-white solid. LCMS (M+H)=835.0; Retention time (10 mM NH₄HCO₃)=3.562 min. ¹H NMR (400 MHz, CDCl₃) δ 7.91 (s, 1H), 7.14 (ddd, J=18.0, 14.6, 6.7 Hz, 10H), 6.31 (dd, J=7.1, 4.3 Hz, 1H), 6.16 (s, 2H), 4.71 (t, J=7.6 Hz, 1H), 4.28-4.20 (m, 3H), 4.02 (t, J=6.7 Hz, 2H), 3.93 (d, J=11.1 Hz, 1H), 3.69 (s, 1H), 2.99 (d, J=6.1 Hz, 2H), 2.73-2.67 (m, 3H), 1.87 (s, 2H), 1.24 (d, J=10.2 Hz, 28H), 0.87 (d, J=6.9 Hz, 3H).

### Example 21

### Octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

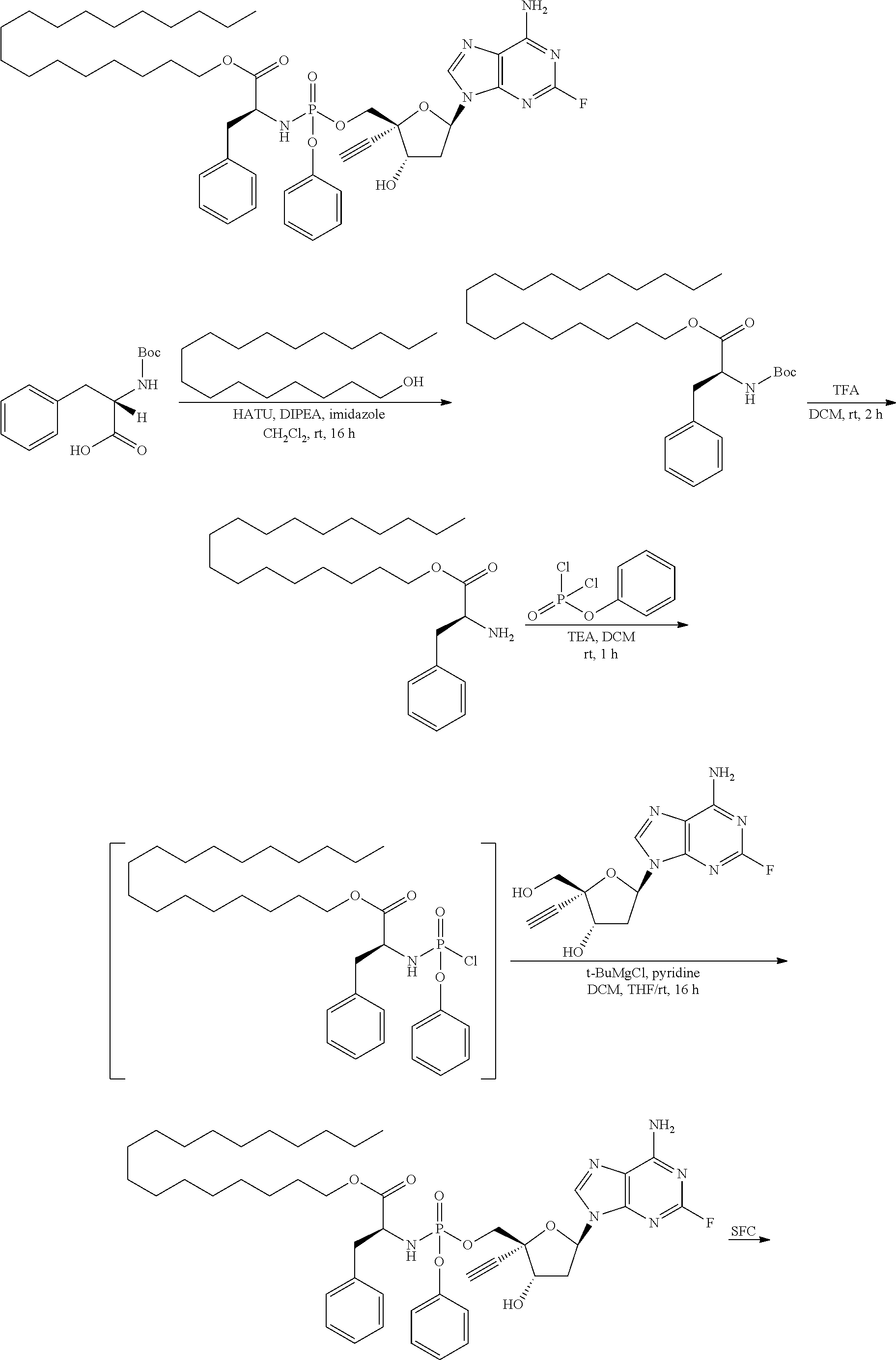

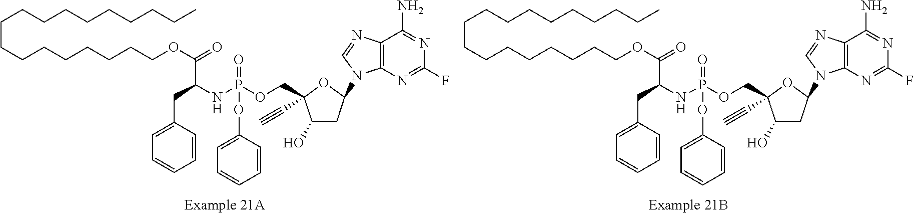 [SMILES: C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1O]

### Step 1: Octadecyl (tert-butoxycarbonyl)phenylalaninate

To a solution of octadecan-1-ol (32.5 g, 120 mmol), (tert-butoxycarbonyl)-L-phenylalanine (29 g, 109 mmol), and HATU (62.3 g, 164 mmol) in DCM (100 mL) was added 1H-imidazole (22.32 g, 328 mmol) and DIPEA (57.3 mL, 328 mmol). The resulting mixture was stirred at 25° C. overnight. TLC (Pet. ether:EtOAc=10:1, Rf=0.5) showed completion of reaction. The reaction was quenched with water (200 mL) and DCM (100 mL). Then, the organic layer was separated and the aqueous layer was extracted with DCM (2×50 mL). The combined organic layers were washed with brine (200 mL), dried with Na₂SO₄, filtered and concentrated to give crude product. The residue was purified by combiflash (silica gel column 330 g, hexane:ethyl acetate=20:1). The appropriate fractions containing desired product were combined and concentrated in vacuum to give octadecyl (tert-butoxycarbonyl)phenylalaninate (32 g, 61.8 mmol, 56.5% yield) as yellow solid.

### Step 2: Octadecyl phenylalaninate

To a solution of octadecyl (tert-butoxycarbonyl)phenylalaninate (32 g, 61.8 mmol) in DCM (400 mL) was added TFA (28.6 mL, 371 mmol). The resulting mixture was stirred at 25° C. for 2 h. TLC (pet. ether:EtOAc=10:1, Rf=0.1) showed the reaction was completed. The reaction was quenched with 1N NaHCO₃ (200 mL) and DCM (100 mL), then the organic layer was separated. The aqueous layer was extracted with DCM (2×100 mL). The combined organic layers were washed with brine (200 mL), dried with Na₂SO₄ and concentrated to give crude product octadecyl phenylalaninate (19 g, 45.5 mmol, 73.6 yield) which was used in the next step without purification.

### Step 3: Octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate

To a solution of octadecyl L-phenylalaninate (5.70 g, 13.64 mmol) and triethylamine (1.901 mL, 13.64 mmol) in DCM (80 mL) was added phenyl phosphorodichloridate (2.038 mL, 13.64 mmol) in DCM (80 mL) dropwise under an atmosphere of nitrogen at about 5° C. Then, the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (2 g, 6.82 mmol) in THF (40 mL)/pyridine (20 mL) was added tert-butylmagnesium chloride (17.05 mL, 17.05 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (40 mL). The combined organic phases were washed with brine, dried over Na₂SO₄, filtered and concentrated to afford the crude product. The residue was purified by reverse phase chromatography (SepaFlash® C18, 0-100% CH₃CN/10 mM NH₄HCO₃ H₂O) to give octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)phenylalaninate (1.2 g, 1.354 mmol, 19.85% yield) as yellow solid. LCMS (M+H)=849.4; Retention time (0.1% TFA)=3.568 min. The diastereomers were separated by Prep-SFC (Instrument: SFC-150 (Waters), Column: IH 20×250 mm, 10 μm (Daicel), Column temperature: 35° C., Mobile phase: CO₂/MeOH (0.2% ammonia/methanol)=60/40, Flow rate: 100 g/min, Back pressure: 100 bar, Detection wavelength: 214 nm, Cycle time: 4 min, Sample solution: 1,900 mg dissolved in 100 mL methanol, Injection volume: 3 mL) to give octadecyl fisrt eluting isomer (Example 21A, RT1: 1.76 min, 800 mg, 0.908 mmol, 38.5% yield); LCMS (M+H)=849.4; Retention time (0.1% TFA)=2.874 min. HPLC: Retention time (0.1% NH₄HCO₃): 11.004 min; ¹H NMR (400 MHz, CDCl₃) δ 7.92 (s, 1H), 7.25-7.17 (m, 5H), 7.14 (d, J=7.3 Hz, 1H), 7.12-7.08 (m, 4H), 6.31 (dd, J=7.3, 4.3 Hz, 1H), 6.10 (s, 2H), 4.71 (t, J=7.6 Hz, 1H), 4.26 (dd, J=11.2, 7.7 Hz, 2H), 4.20 (dd, J=11.3, 8.8 Hz, 1H), 4.03 (t, J=6.7 Hz, 2H), 3.88 (s, 1H), 2.99 (d, J=6.2 Hz, 2H), 2.76-2.63 (m, 3H), 1.86 (s, 2H), 1.24 (d, J=9.0 Hz, 30H), 0.88 (t, J=6.8 Hz, 3H) and second eluting isomer (Example 21B, RT2: 3.21 min, 650 mg, 0.766 mmol, 32.5% yield) as white solid; LCMS (M+H)=849.5; Retention time (0.1% TFA)=2.839 min. HPLC: Retention time (0.1% NH₄HCO₃): 10.931 min; ¹H NMR (400 MHz, CDCl₃) δ 7.78 (s, 1H), 7.30-7.26 (m, 2H), 7.23 (dd, J=8.4, 4.6 Hz, 3H), 7.15 (d, J=7.8 Hz, 3H), 7.06 (dd, J=7.5, 1.7 Hz, 2H), 6.32 (dd, J=7.4, 4.2 Hz, 1H), 6.24 (s, 2H), 4.64 (t, J=7.6 Hz, 1H), 4.23-4.15 (m, 2H), 4.02 (t, J=6.7 Hz, 2H), 3.97 (dd, J=11.5, 8.4 Hz, 1H), 3.73 (t, J=10.8 Hz, 1H), 3.04-2.92 (m, 2H), 2.70 (s, 1H), 2.66 (dt, J=13.7, 7.8 Hz, 1H), 2.57 (ddd, J=13.6, 7.1, 4.2 Hz, 1H), 1.89 (s, 2H), 1.24 (d, J=4.9 Hz, 30H), 0.87 (t, J=6.8 Hz, 3H).

### Example 22

### Pentadecan-8-yl((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

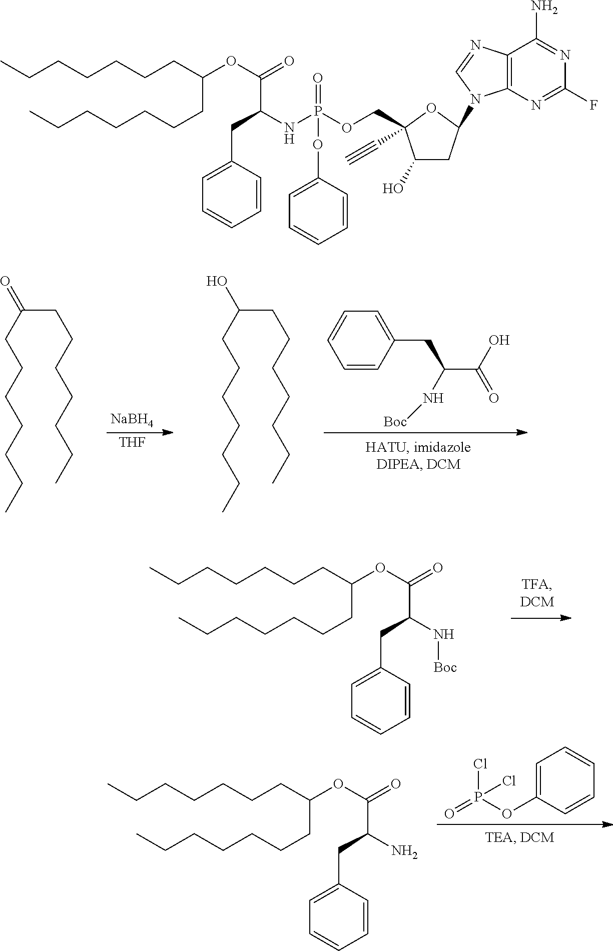

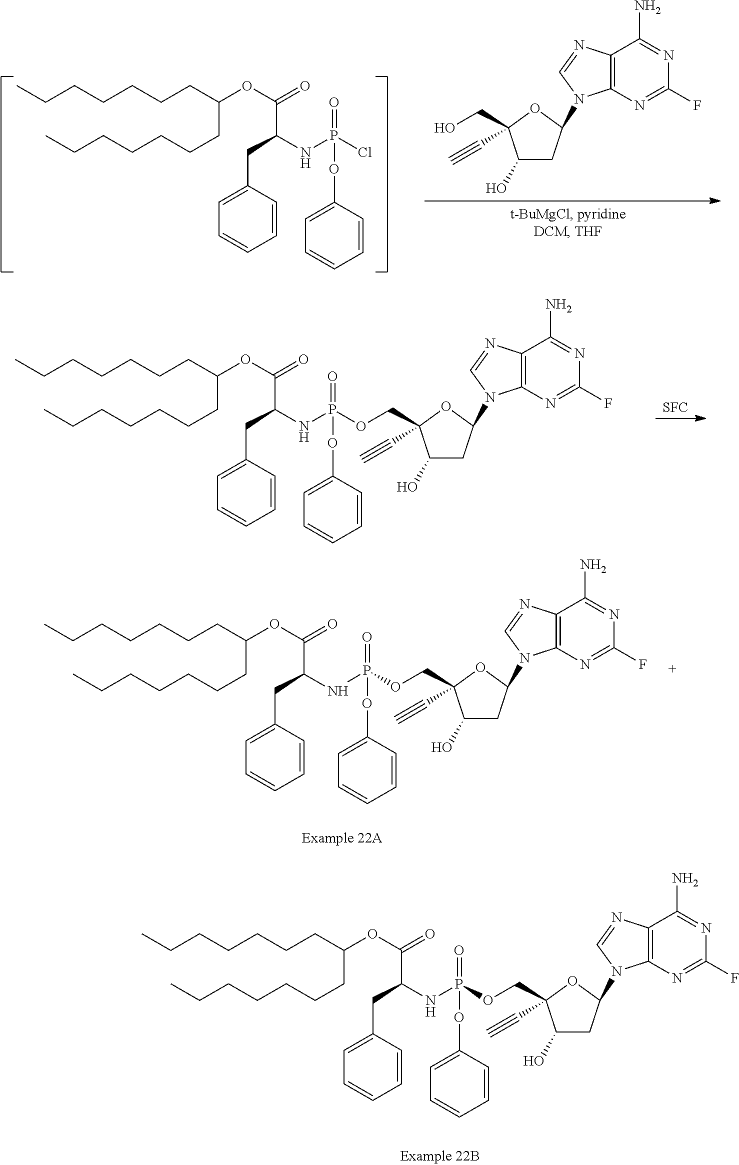

### Step 1: Pentadecan-8-ol

To a solution of pentadecan-8-one (5 g, 22.08 mmol) in THF (50 mL) was added NaBH₄ (1.253 g, 33.1 mmol). The resulting mixture was stirred at 25° C. for 12 h. TLC showed completion of reaction. The reaction was quenched with 2N NH₄Cl (30 mL), organic layer separated and the aqueous layer was extracted with EtOAc (3×40 mL). The combined organic layers were washed with brine (30 mL), dried over Na₂SO₄, filtered and concentrated to afford pentadecan-8-ol (4.1 g, 17.95 mmol, 81% yield). Rf=0.6 (petroleum ether/ethyl acetate=10/1) which was used in the next step without further purification.

### Step 2: Pentadecan-8-yl (tert-butoxycarbonyl)-L-phenylalaninate

To a mixture of pentadecan-8-ol (20.84 g, 91 mmol), (tert-butoxycarbonyl)-L-phenylalanine (22 g, 83 mmol) and HATU (47.3 g, 124 mmol) in DCM (300 mL) were added 1H-imidazole (16.94 g, 249 mmol) and DIPEA (43.4 mL, 249 mmol). The resulting mixture was stirred at 25° C. overnight. LCMS showed completion of reaction. The reaction was quenched with water (200 mL) and organic layer separated. Then, the aqueous layer was extracted with DCM (3×150 mL) and the combined organic layers were washed with brine (200 mL), dried over Na₂SO₄, filtered and concentrated. The residue was purified by combiflash (silica gel column 330 g, petroleum ether/ethyl acetate=20/1). The appropriate fractions containing desired product were combined and concentrated under vacuum to give pentadecan-8-yl (tert-butoxycarbonyl)-L-phenylalaninate (14 g, 29.4 mmol, 35.5 yield) as yellow oil. LCMS (M-Boc+H)=376.3; Retention time (0.05% TFA)=2.13 min.

### Step 3: Pentadecan-8-yl L-phenylalaninate

To a solution of pentadecan-8-yl (tert-butoxycarbonyl)-L-phenylalaninate (12 g, 25.2 mmol) in DCM (100 mL) was added TFA (1.943 mL, 25.2 mmol). The resulting mixture was stirred at 25° C. for 2 h. TLC showed completion of reaction. The reaction was neutralized with NaHCO₃ (50 mL), organic layer separated and the aqueous layer was extracted with DCM (3×100 mL). The combined organic layers were washed with brine (100 mL), dried over Na₂SO₄, filtered and concentrated. The residue was purified by reverse phase chromatography (SepaFlash® C18, 0-100% CH₃CN/10 mM NH₄HCO₃ H₂O) to give pentadecan-8-yl L-phenylalaninate (8 g, 21.30 mmol, 84% yield) as yellow semi-solid. LCMS (M+H)=376.4; Retention time (0.05% TFA)=1.88 min.

### Step 4: Pentadecan-8-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a mixture of pentadecan-8-yl L-phenylalaninate (2.56 g, 6.82 mmol), triethylamine (0.951 mL, 6.82 mmol) in DCM (40 mL) was added phenyl phosphorodichloridate (1.019 mL, 6.82 mmol) in DCM (1 mL) dropwise at 0° C., then the reaction mixture was stirred at the same temperature for 1 h. To a solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (20 mL) and Pyridine (10 mL) was added tert-butylmagnesium chloride (8.52 mL, 8.52 mmol) by dropwise under an atmosphere of nitrogen at 0° C. Then, the mixture was stirred at the same temperature for 30 min. The first prepared mixture was added dropwise to the second prepared mixture at 0° C. and stirred at 0° C. for 2 h. The reaction mixture was quenched with 2N NH₄Cl (10 mL) and diluted EtOAc (200 mL). The organic layer was washed with water (40 mL), brine (40 mL), dried over Na₂SO₄, filtered and concentrated. The residue was purified by reverse phase chromatography (SepaFlash® C18, 0-100% CH₃CN/10 mM NH₄HCO₃ H₂O) to give pentadecan-8-yl ((((2R,3S,5R)-5-(6-amino-2-fluoro-9H-purin-9-yl)-2-ethynyl-3-hydroxytetrahydro-furan-2-yl)methoxy)(phenoxy)phosphoryl)-L-phenylalaninate (150 mg, 0.184 mmol, 5.38% yield) as a white solid. LCMS (M+H)=807.3; Retention time (0.05% TFA)=2.28 min. The diastereomers were separated by Prep-SFC (Instrument: SFC-80 (Thar, Waters), Column: IG 20×250 mm, 10 μm (Daicel), Column temperature: 40° C., Mobile phase: CO₂/EtOH (1.0% ammonia/methanol)=35/65, Flow rate: 80 g/min, Back pressure: 100 bar, Detection wavelength: 214 nm, Cycle time: 14 min, Sample solution: 500 mg dissolved in 35 ml methanol, Injection volume: 2.5 ml) to give first eluting isomer (Example 22A, RT1: 1.9 min, 107 mg, 0.133 mmol, 100%, yield: 21.40%) as a grey solid; LCMS (M+H)=807.0; Retention time (10 mM NH₄HCO₃)=2.68 min. HPLC Retention time (10 mM NH₄HCO₃)=6.79 min. ¹H NMR (400 MHz, CDCl₃) δ 7.91 (s, 1H), 7.25-7.19 (m, 5H), 7.16-7.11 (m, 3H), 7.07 (d, J=8.8 Hz, 2H), 6.31 (dd, J=6.4, 5.2 Hz, 1H), 6.10 (brs, 2H), 4.83 (quint, J=6.0 Hz, 1H), 4.68 (t, J=8.0 Hz, 1H), 4.32-4.24 (m, 1H), 4.23-4.13 (m, 2H), 3.83 (t, J=10.8 Hz, 1H), 3.71 (s, 1H), 3.06 (dd, J=14.0, 6.8 Hz, 1H), 2.95 (dd, J=14.0, 6.8 Hz, 1H), 2.72-2.66 (m, 3H), 1.49-1.40 (m, 4H), 1.26-1.12 (m, 20H), 0.89-0.82 (m, 6H); and second eluting isomer (Example 22B, RT2: 2.8 min, 121 mg, 0.150 mmol, 100%, yield: 24.20%) as a white solid; LCMS (M+H)=807.0; Retention time (10 mM NH₄HCO₃)=2.64 min. HPLC Retention time (10 mM NH₄HCO₃)=6.67 min. ¹H NMR (400 MHz, CDCl₃) δ 7.63 (s, 1H), 7.32-7.27 (m, 3H), 7.25-7.20 (m, 2H), 7.18-7.13 (m, 5H), 6.29 (dd, J=7.6, 4.0 Hz, 1H), 5.88 (brs, 2H), 4.55-4.49 (m, 1H), 4.19-4.11 (m, 1H), 4.07 (dd, J=11.6, 10.0 Hz, 1H), 3.72 (dd, J=12.0, 8.8 Hz, 1H), 3.64-3.55 (m, 2H), 3.07 (ddd, J=13.6, 4.8, 2.4 Hz, 1H), 2.89 (dd, J=13.6, 7.6 Hz, 1H), 2.69 (s, 1H), 2.67-2.59 (m, 1H), 2.47 (ddd, J=13.6, 7.2, 4.0 Hz, 1H), 1.52-1.44 (m, 4H), 1.24-1.12 (m, 20H), 0.88-0.82 (m, 6H).

### Example 23

### Heptadecan-9-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

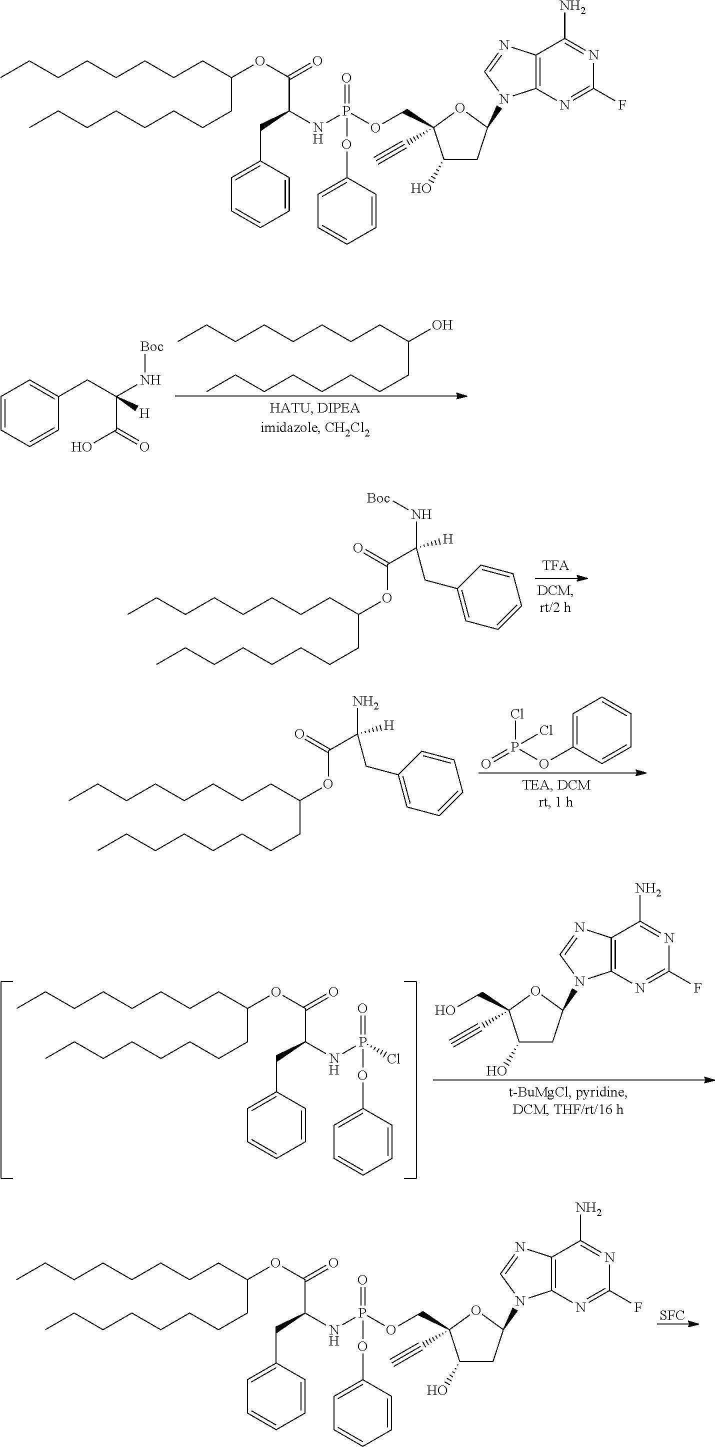

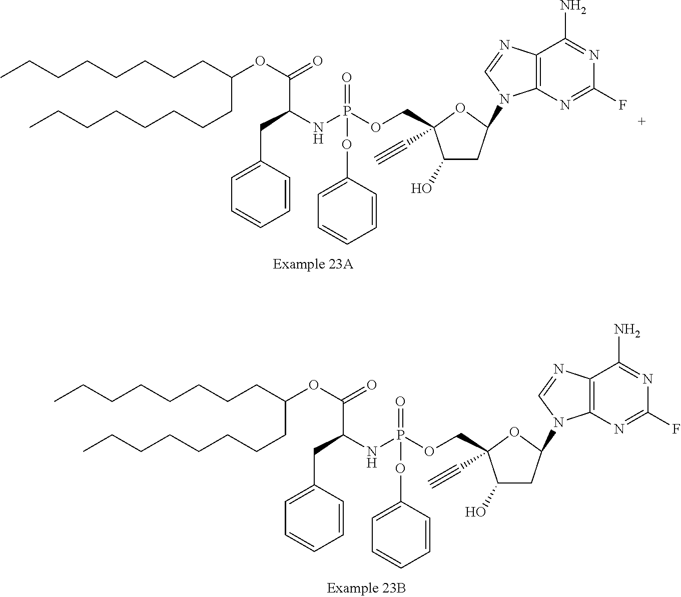 [SMILES: C#C[C@]1(COP(=O)(N[C@@H](Cc2ccccc2)C(=O)OC(CCCCCCCC)CCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OC(CCCCCCCC)CCCCCCCC)Oc2ccccc2)O[C@@H](n2cnc3c(N)nc(F)nc32)C[C@@H]1O]

### Step 1: Heptadecan-9-yl (tert-butoxycarbonyl)-L-phenylalaninate

A mixture of heptadecan-9-ol (12.5 g, 48.7 mmol), (tert-butoxycarbonyl)-L-phenylalanine (12.93 g, 48.7 mmol), imidazole (9.95 g, 146 mmol), DIPEA (25.5 mL, 146 mmol) and HATU (27.8 g, 73.1 mmol) in DCM (120 mL) was stirred at 25° C. for 16 h. LCMS showed the presence of new product. Water (200 mL) was added, and the mixture was extracted with DCM (150 mL×3). The combined organic phases were washed with brine, dried over Na₂SO₄ and concentrated. The residue was purified by silica gel chromatography (pet. ether:EtOAc=50: 1) to afford heptadecan-9-yl (tert-butoxycarbonyl)-L-phenylalaninate (18.8 g, 31.7 mmol, 65.1% yield) as white solid.

### Step 2: Heptadecan-9-yl L-phenylalaninate

A mixture of heptadecan-9-yl (tert-butoxycarbonyl)-L-phenylalaninate (18.8 g, 37.3 mmol) in TFA (30 mL, 389 mmol) and DCM (150 mL) was stirred at 25° C. overnight. LCMS showed completion of reaction. The reaction mixture was concentrated and pH of the residue was adjusted to 8-9 with 1N NaOH and extracted with DCM (100 mL×3). The combined organic phases were washed with brine, dried over Na₂SO₄ and concentrated. The residue was purified by silica gel chromatography (DCM:MeOH=10:1) to afford heptadecan-9-yl L-phenylalaninate (12.2 g, 30.2 mmol, 81% yield) as white solid. LCMS (M+H)=404.2; Retention time (0.1% TFA)=2. 212 min. ¹H NMR (400 MHz, CDCl₃) δ: 7.32-7.21 (m, 5H), 4.90-4.87 (m, 1H), 3.72-3.68 (m, 1H), 3.13-3.09 (m, 1H), 2.84-2.78 (m, 1H), 1.55-1.48 (m, 4H), 1.29-1.25 (m, 24H), 0.89-0.86 (m, 6H).

### Step 3: Heptadecan-9-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of heptadecan-9-yl L-phenylalaninate (2.75 g, 6.82 mmol), triethylamine (0.951 mL, 6.82 mmol) in DCM (60 mL) was added phenyl phosphorodichloridate (1.019 mL, 6.82 mmol) in DCM (1 mL) dropwise under an atmosphere of nitrogen at about 5° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (20 mL)/pyridine (10 mL) was added tert-butylmagnesium chloride (8.52 mL, 8.52 mmol) dropwise under an atmosphere of nitrogen at about 5° C., then the mixture was stirred at room temperature for 30 min. The first prepared mixture was slowly added to the second prepared mixture at room temperature and stirred for 2 h. LCMS showed completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (30×2 mL). The combined organic layers were washed with brine, dried over Na₂SO₄, filtered and concentrated to afford the crude product. The residue was purified by reverse phase chromatography (SepaFlash® C18, 0-100% CH₃CN/10 mM NH₄HCO₃ H₂O) to give heptadecan-9-yl ((((2R,3S,5R)-5-(6-amino-2-fluoro-9H-purin-9-yl)-2-ethynyl-3-hydroxytetrahydrofuran yl)methoxy)(phenoxy)phosphoryl)-L-phenylalaninate (600 mg, 0.660 mmol, 19.36% yield) as light-yellow solid. LCMS (M+H)=835.4; Retention time (0.1% TFA)=2.474 and 2.506 min. The diastereomers were separated by Prep-SFC (Instrument: SFC-150 (Waters), Column: OX 20×250 mm, 10 μm (Daicel), Column temperature: 35° C., Mobile phase: CO₂/ETOH(0.5% ammonia/methanol)=65/35; Flow rate: 100 g/min; Back pressure: 100 bar; Detection wavelength: 214 nm; Cycle time: 5 min; Sample solution: 700 mg dissolved in 60 ml methanol; Injection volume: 3 mL) to afford first eluting isomer (Example 23A, RT1: 1.13 min, 109 mg, 0.125 mmol, 17.43% yield); LCMS (M+H)=835.4; Retention time (0.1% TFA)=2.468 min. HPLC: Retention time (0.1% NH₄HCO₃)=9.409 min; ¹H NMR (400 MHz, CDCl₃) δ 7.92 (s, 1H), 7.25-7.17 (m, 5H), 7.14 (d, J=7.3 Hz, 1H), 7.12-7.08 (m, 4H), 6.31 (dd, J=7.3, 4.3 Hz, 1H), 6.10 (s, 2H), 4.71 (t, J=7.6 Hz, 1H), 4.26 (dd, J=11.2, 7.7 Hz, 2H), 4.20 (dd, J=11.3, 8.8 Hz, 1H), 4.03 (t, J=6.7 Hz, 2H), 3.88 (s, 1H), 2.99 (d, J=6.2 Hz, 2H), 2.76-2.63 (m, 3H), 1.86 (s, 2H), 1.24 (d, J=9.0 Hz, 30H), 0.88 (t, J=6.8 Hz, 3H). and second eluting isomer (Example 23B, RT2: 2.12 min, 145 mg, 0.167 mmol, 23.29% yield) as white solid; LCMS (M+H)=835.3; Retention time (0.1% TFA)=2.505 min. HPLC: Retention time (0.1% NH₄HCO₃)=9.584 min. ¹H NMR (400 MHz, CDCl₃) δ 7.78 (s, 1H), 7.30-7.26 (m, 2H), 7.23 (dd, J=8.4, 4.6 Hz, 3H), 7.15 (d, J=7.8 Hz, 3H), 7.06 (dd, J=7.5, 1.7 Hz, 2H), 6.32 (dd, J=7.4, 4.2 Hz, 1H), 6.24 (s, 2H), 4.64 (t, J=7.6 Hz, 1H), 4.23-4.15 (m, 2H), 4.02 (t, J=6.7 Hz, 2H), 3.97 (dd, J=11.5, 8.4 Hz, 1H), 3.73 (t, J=10.8 Hz, 1H), 3.04-2.92 (m, 2H), 2.70 (s, 1H), 2.66 (dt, J=13.7, 7.8 Hz, 1H), 2.57 (ddd, J=13.6, 7.1, 4.2 Hz, 1H), 1.89 (s, 2H), 1.24 (d, J=4.9 Hz, 30H), 0.87 (t, J=6.8 Hz, 3H).

### Example 24

### Nonadecan-10-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

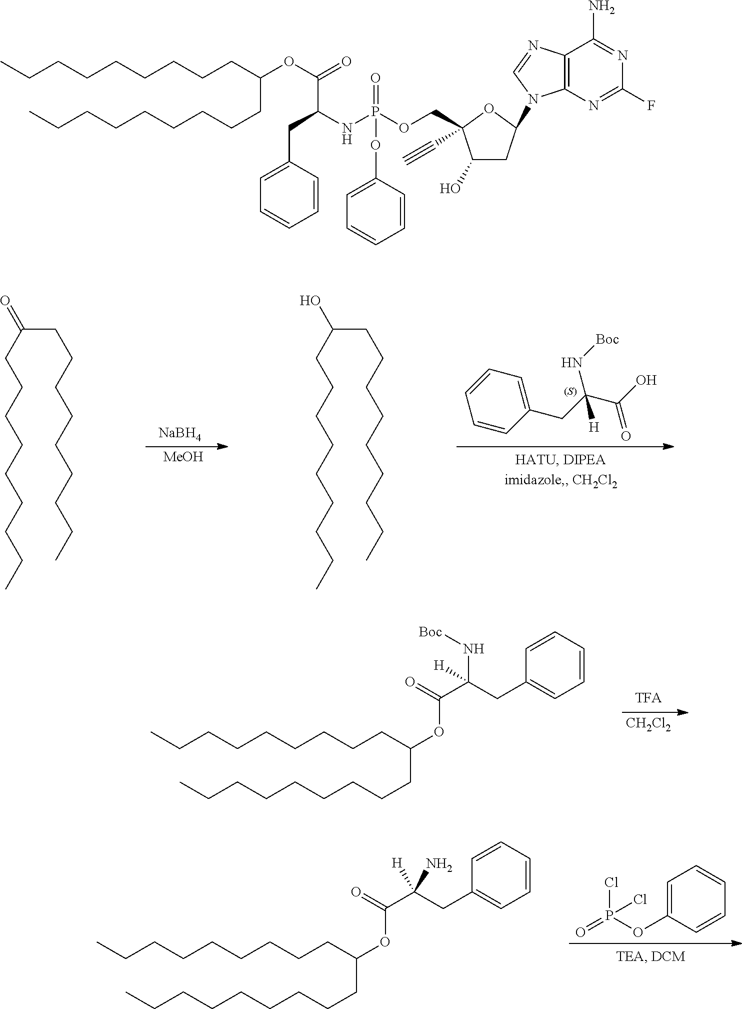

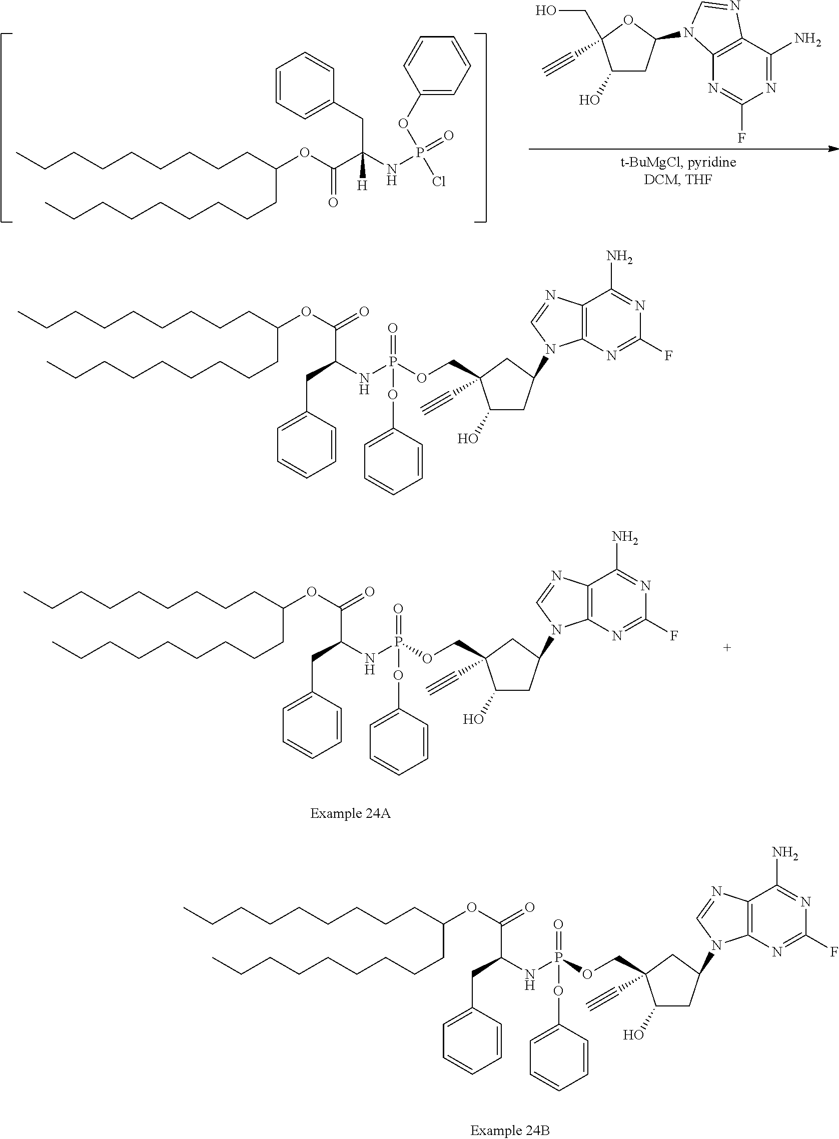

### Step 1: Nonadecan-10-ol

To a solution of nonadecan-10-one (24 g, 85 mmol) in methanol (300 mL) and THF (1,000 mL) was added NaBH₄ (4.82 g, 127 mmol) at 0° C. The reaction mixture was stirred at 25° C. for 2 h and quenched with slow addition of 200 (mL) sat. NH₄Cl and concentrated. Then, diluted with H₂O (500 mL) and EtOAc (600 mL). The organic phase was separated and the aqueous layer was extracted with EtOAc (200 ml×2). The combined organic layers were washed with water (200 ml×2), dried over Na₂SO₄ and concentrated to give nonadecan-10-ol (23 g, 81 mmol, 95% yield) as white solid which was used in the next step without purification. LCMS: no mass.

### Step 2: Nonadecan-10-yl (tert-butoxycarbonyl)-L-phenylalaninate

To a solution of (tert-butoxycarbonyl)-L-phenylalanine (11 g, 41.5 mmol) in DCM (250 mL) was added nonadecan-10-ol (12.39 g, 43.5 mmol), imidazole (8.47 g, 124 mmol), DIPEA (21.72 mL, 124 mmol) and HATU (23.65 g, 62.2 mmol) and the resulting mixture was stirred at 25° C. for 16 h. TLC showed presence of new compound. The reaction mixture was diluted with water (200 mL), organic layer separated and the aqueous layer extracted with DCM (150 mL×3). The combined organic phases were washed with brine, dried over Na₂SO₄ and concentrated. The residue was purified by silica gel chromatography (pet. ether:EtOAc=20:1) to afford nonadecan-10-yl (tert-butoxycarbonyl)-L-phenylalaninate (28 g, 26.3 mmol, 63.5% yield) as a white solid.

### Step 3: Nonadecan-10-yl L-phenylalaninate

To a solution of nonadecan-10-yl (tert-butoxycarbonyl)-L-phenylalaninate (18 g, 33.8 mmol) in DCM (80 mL) was added trifluoroacetic acid (20 mL) at 0° C. The resulting mixture was stirred at room temperature for 2 h. TLC showed completion of reaction. The solvents was removed under reduced pressure and the crude purified by silica gel chromatography (40 g, pet. ether:EtOAc=20:1 then DCM:MeOH=20:1) to give nonadecan-10-yl L-phenylalaninate (14 g, 29.2 mmol, 86% yield) as an oil.

### Step 4: Nonadecan-10-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of nonadecan-10-yl L-phenylalaninate (1,840 mg, 4.26 mmol), triethylamine (0.594 mL, 4.26 mmol) in DCM (30 mL) was added phenyl phosphorodichloridate (0.510 mL, 3.41 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (500 mg, 1.705 mmol) in THF (20 mL)/pyridine (10 mL) was added tert-butylmagnesium chloride (4.26 mL, 4.26 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. The solvent was removed under reduced pressure and residue was diluted with EtOAc (200 mL) and water (50 mL). The organic phase was separated and the aqueous layer was extracted with EtOAc (20 ml×2). The combined organic phases were washed with water (50 ml×2), dried over Na₂SO₄ and concentrated. The residue was purified by gel silica chromatography (80 g, MeOH:DCM=1:15) to give nonadecan-10-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (650 mg, 0.742 mmol, 43.5% yield) as a solid. LCMS (M+H)=863.2; Retention time (0.1% TFA)=2.77, 2.83 min. The diastereomers were separated by Prep-SFC (Instrument: SFC-80 (Thar, Waters); Column: OZ 20×250 mm, 10 μm (Daicel); Column temperature: 40° C.; Mobile phase: CO₂/MeOH (0.2% ammonia/methanol)=45/55; Flow rate: 80 g/min; Back pressure: 100 bar; Detection wavelength: 260 nm; Cycle time: 7.5 min; Sample solution: 356 mg dissolved in 10 mL methanol; Injection volume: 1.5 mL) to give first eluting isomer (Example 24A, RT1: 1.71 min, 184.6 mg, 0.217 mmol, 99.36%, yield: 26.8%) as an off-white solid; LCMS (M+H)=863.2; Retention time (0.1% TFA)=3.39 min. ¹H NMR (400 MHz, DMSO) δ 8.22 (s, 1H), 7.85 (brs, 2H), 7.27-7.19 (m, 4H), 7.18-7.08 (m, 4H), 7.04 (d, J=8.4 Hz, 2H), 6.23 (dd, J=7.6, 4.9 Hz, 1H), 6.09 (dd, J=11.9, 10.9 Hz, 1H), 5.73 (d, J=5.6 Hz, 1H), 4.68-4.60 (m, 1H), 4.51 (dd, J=12.8, 7.0 Hz, 1H), 4.03-3.92 (m, 2H), 3.75 (dd, J=10.9, 4.4 Hz, 1H), 3.61 (s, 1H), 2.98-2.89 (m, 1H), 2.78 (dd, J=13.5, 8.3 Hz, 1H), 2.74-2.66 (m, 1H), 2.46-2.39 (m, 1H), 1.31-1.00 (m, 32H), 0.83 (td, J=6.9, 3.1 Hz, 6H) and second eluting isomer (Example 24B, RT2: 2.41 min, 133.7 mg, 0.155 mmol, 98.94%, yield: 19.1%) as an off-white solid. LCMS (M+H)=863.5; Retention time (0.1% TFA)=3.34 min. ¹H NMR (400 MHz, DMSO) δ 8.17 (s, 1H), 7.85 (s, 2H), 7.25-7.16 (m, 5H), 7.15-7.07 (m, 3H), 6.99 (d, J=8.4 Hz, 2H), 6.26 (dd, J=7.3, 5.4 Hz, 1H), 6.13 (dd, J=13.1, 10.6 Hz, 1H), 5.76 (d, J=5.6 Hz, 1H), 4.66 (p, J=6.0 Hz, 1H), 4.50 (dd, J=12.5, 6.7 Hz, 1H), 3.99 (dd, J=10.7, 5.2 Hz, 1H), 3.95-3.85 (m, 2H), 3.64 (s, 1H), 2.95-2.87 (m, 1H), 2.80 (dd, J=13.5, 8.0 Hz, 1H), 2.70-2.62 (m, 1H), 2.46-2.37 (m, 1H), 1.30-0.98 (m, 32H), 0.83 (td, J=6.9, 4.7 Hz, 6H).

### Example 25

### Henicosan-11-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

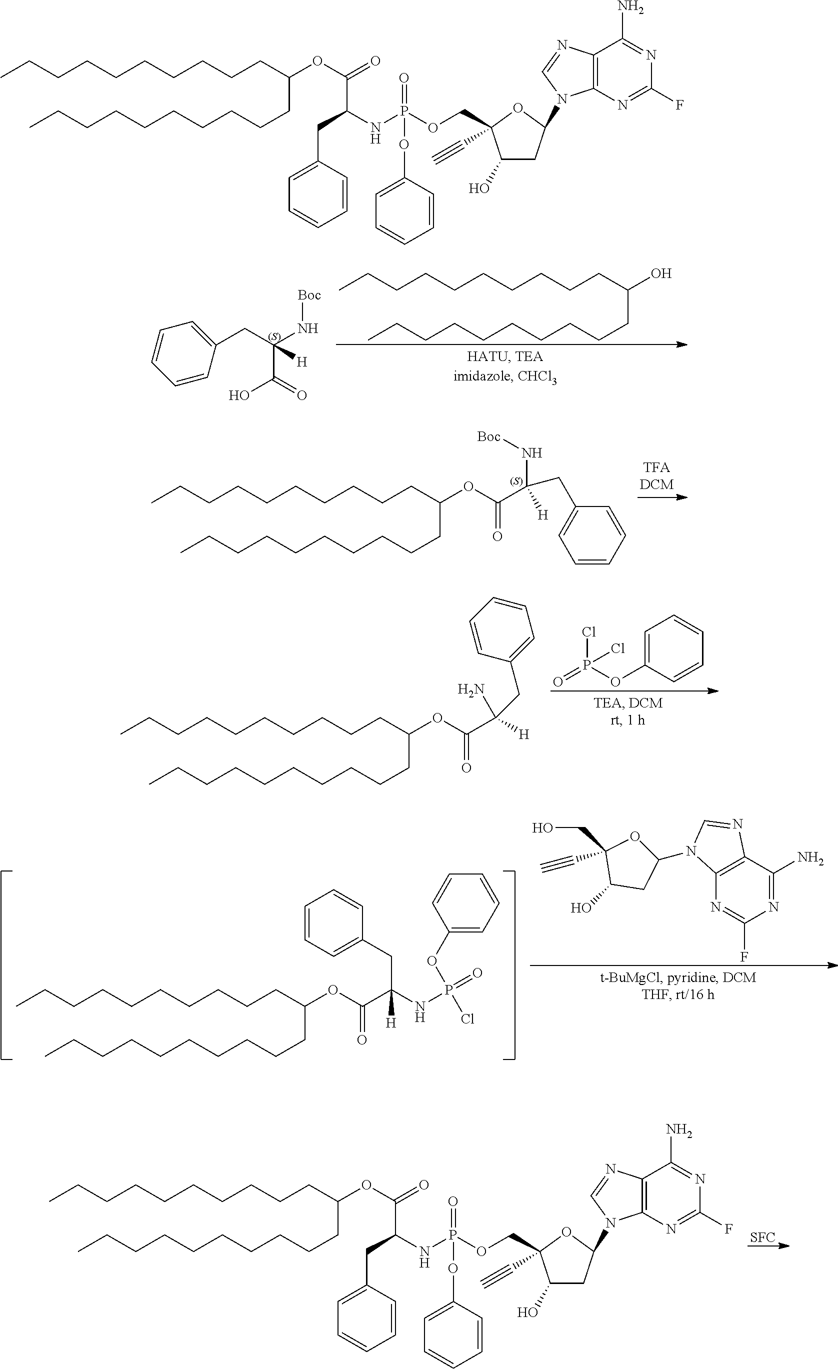

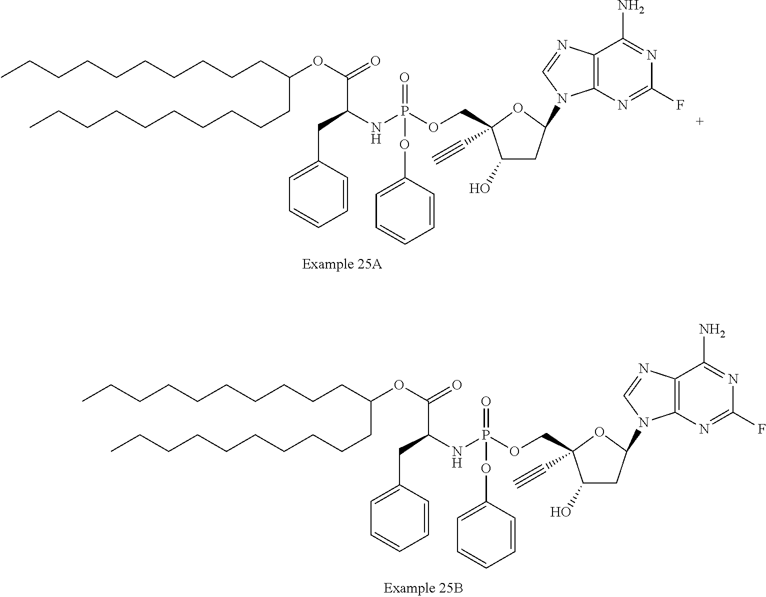 [SMILES: C#C[C@]1(COP(=O)(N[C@@H](Cc2ccccc2)C(=O)OC(CCCCCCCCCC)CCCCCCCCCC)Oc2ccccc2)O[C@@H](n2cnc3c(N)nc(F)nc32)C[C@@H]1O ; C#C[C@]1(COP(=O)(N[C@@H](Cc2ccccc2)C(=O)OC(CCCCCCCCCC)CCCCCCCCCC)Oc2ccccc2)O[C@@H](n2cnc3c(N)nc(F)nc32)C[C@@H]1O]

### Step 1: Henicosan-11-yl (tert-butoxycarbonyl)-L-phenylalaninate

A mixture of henicosan-11-ol (10 g, 32.0 mmol), (tert-butoxycarbonyl)-L-phenylalanine (8.49 g, 32.0 mmol), imidazole (6.53 g, 96 mmol), DIPEA (16.76 mL, 96 mmol) and HATU (18.25 g, 48.0 mmol) in DCM (120 mL) was stirred at 25° C. for 16 h. TLC (pet. ether:EtOAc=20:1, Rf=0.5) showed presence of new product. The reaction mixture was diluted with water (200 mL), organic layer separated and the aqueous layer was extracted with DCM (150 mL×3). The combined organic phases were washed with brine, dried over Na₂SO₄ and concentrated. The residue was purified by silica gel chromatography (pet. ether:EtOAc=50:1) to give henicosan-11-yl (tert-butoxycarbonyl)-L-phenylalaninate (13 g, 19.74 mmol, 61.7% yield) as white solid. LCMS (M+Na)=581.1; Retention time (0.1% TFA)=1.887 min.

### Step 2: Henicosan-11-yl L-phenylalaninate

A mixture of henicosan-11-yl(tert-butoxycarbonyl)-L-phenylalaninate (13 g, 23.22 mmol) in TFA (20 mL, 260 mmol) and DCM (100 mL) was stirred at 25° C. overnight. LCMS showed the presence of new product. The reaction was concentrated and pH of the residue was adjusted to 8-9 with 1N NaOH. The organic layer was separated and the aqueous layer extracted with DCM (100 mL×3). The combined organic phases were washed with brine, dried over Na₂SO₄ and concentrated. The residue was purified by silica gel chromatography (DCM:MeOH=10:1) to afford henicosan-11-yl L-phenylalaninate (6.28 g, 13.66 mmol, 58.8% yield) as white solid. LCMS (M+H)=460.3; Retention time (0.1% TFA)=2.411 min. ¹H NMR (400 MHz, CDCl₃) δ: 7.30-7.21 (m, 5H), 4.89-4.87 (m, 1H), 3.72-3.68 (m, 1H), 3.10-3.09 (m, 1H), 2.84-2.82 (m, 1H), 1.54-1.48 (m, 4H), 1.29-1.25 (m, 32H), 0.89-0.86 (m, 6H).

### Step 3: Henicosan-11-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of henicosan-11-yl L-phenylalaninate (1.254 g, 2.73 mmol), triethylamine (0.380 mL, 2.73 mmol) in DCM (20 mL) was added phenyl phosphorodichloridate (0.408 mL, 2.73 mmol) in DCM (1 mL) dropwise under an atmosphere of nitrogen at about 5° C. Then, the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (0.4 g, 1.364 mmol) in THF (20 mL)/pyridine (10 mL) was added tert-butylmagnesium chloride (3.41 mL, 3.41 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (30 mL×2). The combined organic phases were washed with brine, dried over Na₂SO₄, filtered and concentrated to afford the crude product. The residue was purified by reverse phase chromatography (SepaFlash® C18, 0-100% CH₃CN/10 mM NH₄CO₃ H₂O) to obtain henicosan-11-yl((((2R,3S,5R)-5-(6-amino-2-fluoro-9H-purin-9-yl)-2-ethynyl-3-hydroxytetrahydrofuran yl)methoxy)(phenoxy)phosphoryl)-L-phenylalaninate (240 mg, 0.256 mmol, 18.77% yield). LCMS: no mass, Retention time (0.1% TFA)=4.004, 4.097 min. The diastereomers were separated by two successive Prep-SFC purifications. First time with Instrument: SFC-150 (Waters), Column: OX 20×250 mm, 10 μm (Daicel), Column temperature: 35° C., Mobile phase: CO₂/MeOH (0.2% ammonia/methanol)=55/45, Flow rate: 100 g/min, Back pressure: 100 bar, Detection wavelength: 214 nm, Cycle time: 4.5 min, Sample solution: 280 mg dissolved in 40 ml methanol; Injection volume: 2 mL) and second time with Instrument: SFC-150 (Waters), Column: ID 20×250 mm, 10 μm (Daicel), Column temperature: 35° C., Mobile phase: CO₂/MeOH (0.2% ammonia/methanol)=55/45, Flow rate: 120 g/min, Back pressure: 100 bar, Detection wavelength: 214 nm, Cycle time: 5 min, Sample solution: 220 mg, dissolved in 40 mL methanol, Injection volume: 2 mL) to obtain first eluting isomer (Example 25A, RT1: 1.08 min, 133 mg, 0.145 mmol, 32.3% yield); LCMS: Retention time (0.1% TFA)=3.697 min. HPLC: Retention time (0.1% NH₄HCO₃)=12.652 min; ¹H NMR (400 MHz, CDCl₃) δ 7.64 (s, 1H), 7.29 (dd, J=11.0, 6.9 Hz, 3H), 7.25-7.20 (m, 2H), 7.18-7.13 (m, 5H), 6.29 (dd, J=7.6, 4.1 Hz, 1H), 5.86 (s, 2H), 4.90-4.82 (m, 1H), 4.51 (t, J=7.7 Hz, 1H), 4.19-4.11 (m, 1H), 4.70 (dd, J=11.8, 10.0 Hz 1H), 3.72 (dd, J=11.7, 9.3 Hz, 1H), 3.63-3.38 (m, 2H), 3.07 (d, J=10.8 Hz, 1H), 2.89 (dd, J=13.6, 7.7 Hz, 1H), 2.69 (s, 1H), 2.67-2.58 (m, 1H), 2.51-2.43 (m, 1H), 1.66 (s, 6H), 1.25-1.15 (m, 30H), 0.87 (dd, J=6.9, 5.8 Hz, 6H) and second eluting isomer (Example 25B, RT2: 1.69 min, 100 mg, 0.107 mmol, 23.77% yield); LCMS: Retention time (0.1% TFA)=3.785 min. HPLC: Retention time (0.1% NH₄HCO₃)=13.150 min; ¹H NMR (400 MHz, CDCl₃) δ 7.90 (s, 1H), 7.25-7.17 (m, 5H), 7.13 (t, J=6.8 Hz, 3H), 7.07 (d, J=8.0 Hz, 2H), 6.30 (t, J=5.7 Hz, 1H), 6.21 (s, 2H), 4.85-4.78 (m, 1H), 4.67 (dd, J=12.8, 7.8 Hz, 1H), 4.32-4.25 (m, 1H), 4.25-4.13 (m, 2H), 3.90 (t, J=10.7 Hz, 1H), 3.80 (d, J=4.2 Hz, 1H), 3.06 (dd, J=12.8, 5.6 Hz, 1H), 2.94 (dd, J=13.9, 7.2 Hz, 1H), 2.71-2.65 (m, 3H), 1.92 (s, 4H), 1.25-1.15 (m, 32H), 0.87 (t, J=7.2 Hz, 6H).

### Example 26

### Tricosan-12-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

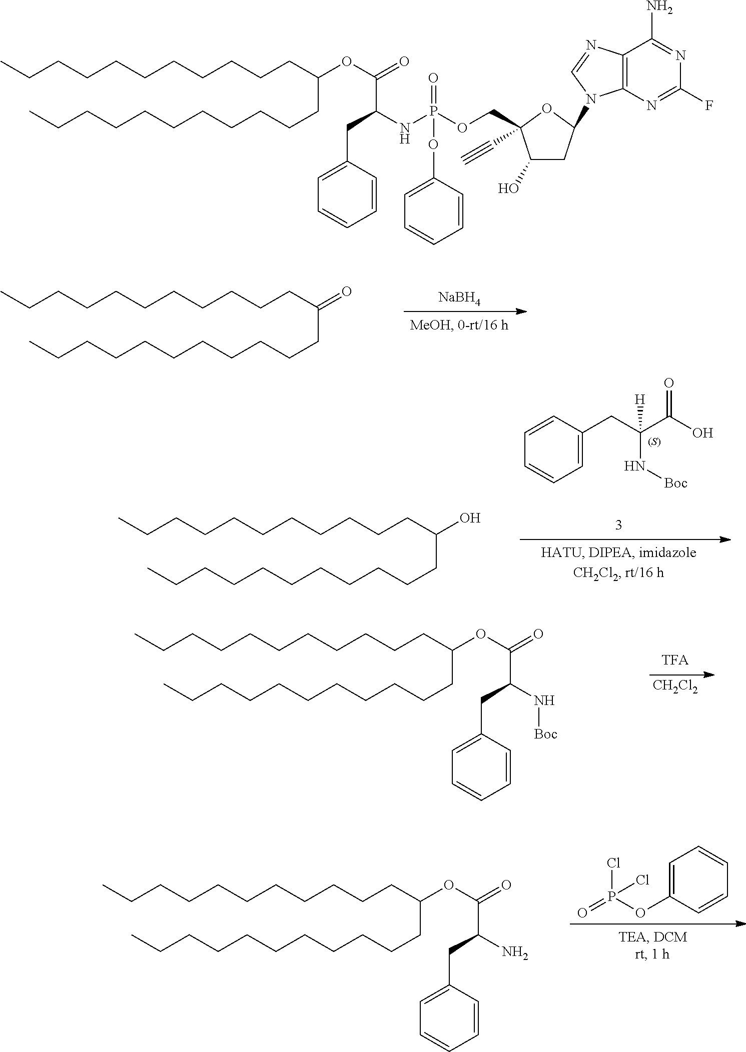

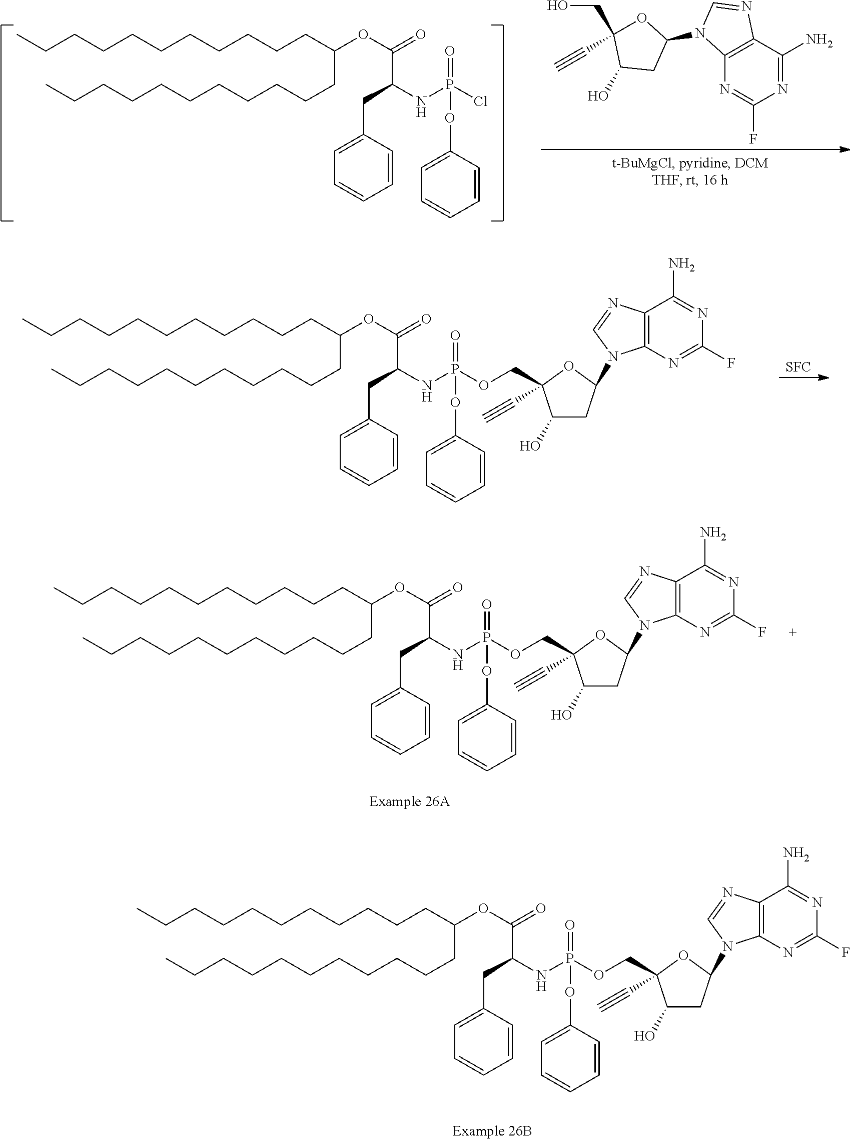

### Step 1: Tricosan-12-ol

To a solution of tricosan-12-one (50 g, 148 mmol) in THF (500 mL) and methanol (83 mL) was added NaBH₄ (8.38 g, 221 mmol) slowly at 0° C. The reaction mixture was stirred at room temperature for 16 h. TLC showed the reaction was completed. The mixture was carefully quenched with NH₄Cl solution and concentrated to remove organic solvent. The aqueous reaction mixture was extracted twice with EtOAc (500 mL) and combined organic phases were washed with water (300 ml), brine (300 mL), dried over Na₂SO₄, concentrated to give tricosan-12-ol (48 g, 141 mmol, 95% yield) as white solid.

### Step 2: Tricosan-12-yl (tert-butoxycarbonyl)-L-phenylalaninate

To a mixture of tricosan-12-ol (32 g, 94 mmol), (tert-butoxycarbonyl)-L-phenylalanine (24.92 g, 94 mmol), 1H-imidazole (19.19 g, 282 mmol) and HATU (53.6 g, 141 mmol) in DCM (1,000 mL) was added DIPEA (49.2 mL, 282 mmol) and the resulting mixture was stirred at room temperature for 16 h. TLC (pet. ether:EtOAc=20:1, Rf=0.7) showed the reaction was completed. The mixture was washed with water and dried over Na₂SO₄, and concentrated to give crude product. The crude product was purified by flash silica gel chromatography (pet. ether:EtOAc=20:1) to give tricosan-12-yl (tert-butoxycarbonyl)-L-phenylalaninate (30 g, 51.0 mmol, 54.3% yield) as a white solid.

### Step 3: Tricosan-12-yl L-phenylalaninate

To a solution of tricosan-12-yl (tert-butoxycarbonyl)-L-phenylalaninate (30 g, 51.0 mmol) in DCM (500 mL) was added trifluoroacetic acid (100 mL). The resulting mixture was stirred at room temperature for 12 h. TLC (pet. ether:EtOAc=5:1, Rf=0.3) showed the reaction was completed. The reaction mixture was concentrated to remove DCM and pH of the residue was adjusted with NaHCO₃ (aq) to ˜8 and extracted with EtOAc (2×100 mL). The combined organic phases were washed with water (300 ml), brine (300 mL), dried over Na₂SO₄ and concentrated to give crude product. The crude product was purified by flash silica gel chromatography (pet. ether:EtOAc=5:1) to give tricosan-12-yl L-phenylalaninate (19 g, 38.9 mmol, 76% yield) as a yellow oil.

### Step 4: Tricosan-12-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of tricosan-12-yl L-phenylalaninate (3.99 g, 8.18 mmol), triethylamine (1.141 mL, 8.18 mmol) in DCM (50 mL) was added phenyl phosphorodichloridate (1.223 mL, 8.18 mmol) in DCM (1 mL) dropwise under an atmosphere of nitrogen at about 5° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (1.2 g, 4.09 mmol) in THF (20 mL)/pyridine (10 mL) was added tert-butylmagnesium chloride (10.23 mL, 10.23 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (2×30 mL). The combined organic phases were washed with brine, dried over Na₂SO₄, filtered and concentrated to afford the crude product. The residue was purified by reverse phase chromatography (SepaFlash® C18, 0-100% CH₃CN/10 mM NH₄HCO₃ H₂O) to give tricosan-12-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (650 mg, 0.696 mmol, 17.02% yield). LCMS: Retention time (0.1% TFA)=4.242, 4.352 min. The diastereomers were separated by Prep-SFC ((Instrument: SFC-80 (Thar, Waters) Column: AD 20×250 mm, 10 μm (Daicel), Column temperature: 40° C. Mobile phase: CO₂/MeOH(1.0% ammonia/methanol)=40/60, Flow rate: 80 g/min Back pressure: 100 bar, Detection wavelength: 214 nm Cycle time: 9.6 min)) to give first eluting isomer (Example 26A, RT1: 1.89 min, 117 mg, 0.126 mmol, 24.11% yield); LCMS (M+H)=919.5; Retention time (0.1% TFA)=4.386 min. HPLC: Retention time (0.1% NH₄HCO₃): 13.113 min; ¹H NMR (400 MHz, CDCl₃) δ 7.90 (s, 1H), 7.25-7.16 (m, 5H), 7.12 (dd, J=14.8, 7.9 Hz, 3H), 7.07 (d, J=7.7 Hz, 2H), 6.40-5.96 (m, 3H), 4.85-4.77 (m, 1H), 4.68 (t, J=7.3 Hz, 1H), 4.31-4.12 (m, 3H), 4.00-3.71 (m, 2H), 3.06 (dd, J=13.9, 5.7 Hz, 1H), 2.94 (dd, J=13.8, 7.0 Hz, 1H), 2.72-2.64 (m, 3H), 1.89 (d, J=26.0 Hz, 4H), 1.28-1.15 (m, 36H), 0.87 (t, J=6.8 Hz, 6H) and second eluting isomer (Example 26B, RT2: 2.35 min, 130 mg, 0.139 mmol, 26.7% yield); LCMS: Retention time (0.1% TFA)=4.373 min. HPLC: Retention time (0.1% NH₄HCO₃)=14.360 min; ¹H NMR (400 MHz, CDCl₃) δ 7.67 (s, 1H), 7.29 (d, J=7.9 Hz, 2H), 7.25-7.19 (m, 3H), 7.14 (dd, J=5.1, 2.7 Hz, 5H), 6.29 (dd, J=7.5, 4.0 Hz, 1H), 6.12 (s, 2H), 4.88-4.83 (m, 1H), 4.51 (t, J=7.6 Hz, 1H), 4.18-4.11 (m, 1H), 4.08 (dd, J=11.8, 9.9 Hz, 1H), 3.77-3.69 (m, 3H), 3.10-3.04 (m, 1H), 2.89 (dd, J=13.7, 7.7 Hz, 1H), 2.68 (s, 1H), 2.63 (dt, J=13.6, 7.9 Hz, 1H), 2.46 (ddd, J=13.6, 7.2, 4.1 Hz, 1H), 1.88 (s, 4H), 1.25-1.15 (m, 36H), 0.87 (dd, J=7.0, 6.2 Hz, 6H).

### Example 27

### Tridecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

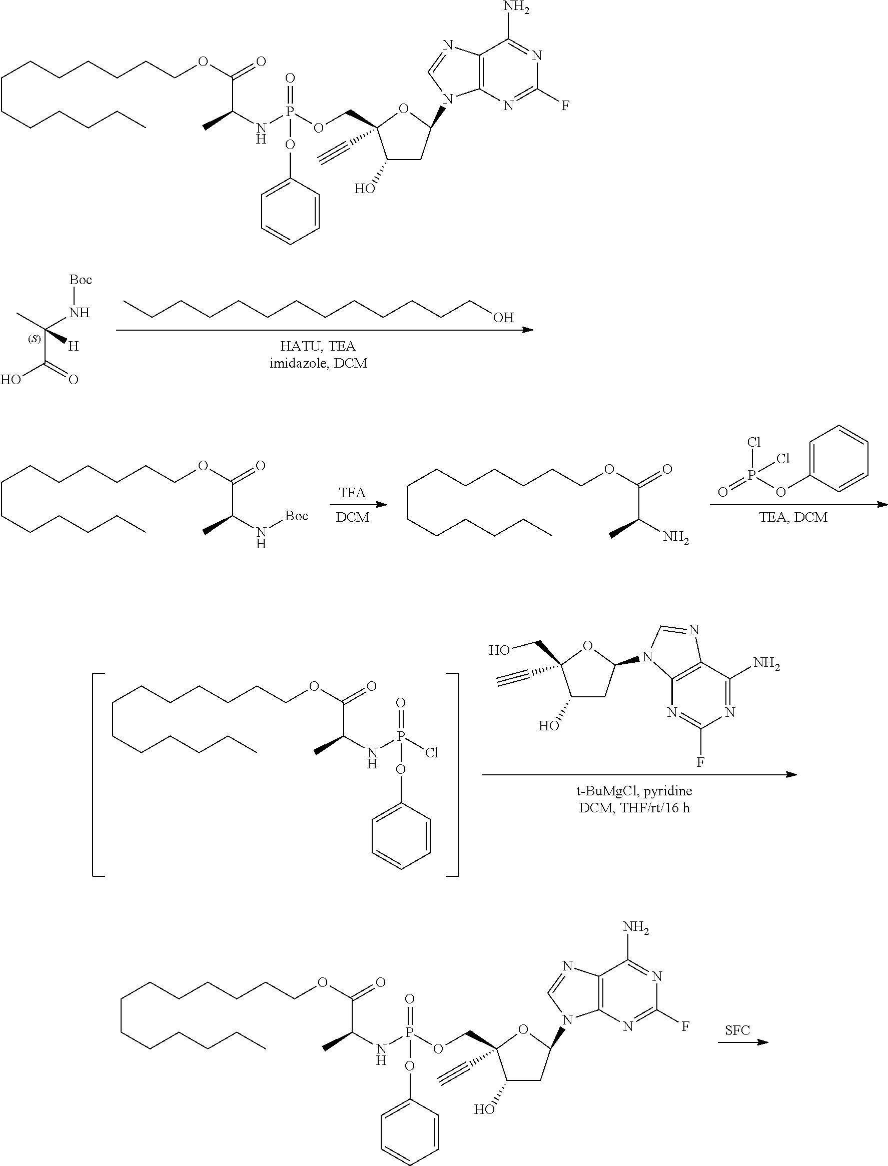

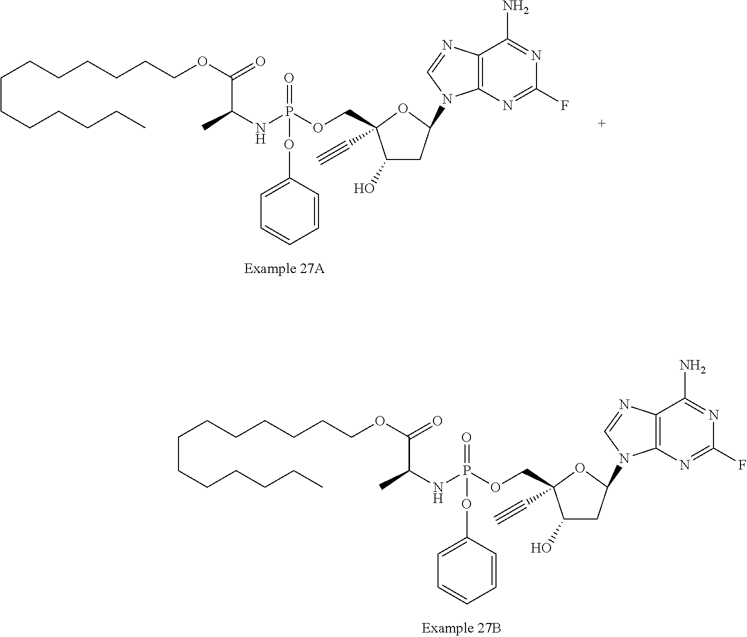 [SMILES: C#C[C@]1(COP(=O)(N[C@@H](C)C(=O)OCCCCCCCCCCCCC)Oc2ccccc2)O[C@@H](n2cnc3c(N)nc(F)nc32)C[C@@H]1O ; C#C[C@]1(COP(=O)(N[C@@H](C)C(=O)OCCCCCCCCCCCCC)Oc2ccccc2)O[C@@H](n2cnc3c(N)nc(F)nc32)C[C@@H]1O]

### Step 1: Tridecyl (tert-butoxycarbonyl)-L-alaninate

A mixture of (tert-butoxycarbonyl)-L-alanine (9.44 g, 49.9 mmol), tridecan-1-ol (10 g, 49.9 mmol), imidazole (10.19 g, 150 mmol), DIPEA (26.2 mL, 150 mmol) and HATU (28.5 g, 74.9 mmol) in DCM (100 mL) was stirred at 25° C. for 16 h. TLC showed the presence of new compound. Water (200 mL) was added and the mixture was extracted with DCM (150 mL×3). The combined organic phases were washed with brine, dried over Na₂SO₄ and concentrated. The residue was purified by silica gel chromatography (pet. ether:EtOAc=30:1) to afford tridecyl (tert-butoxycarbonyl)-L-alaninate (6.8 g, 18.30 mmol, 36.7% yield) as white solid. LCMS (M+Na)=394.3; Retention time (10 mM NH₄HCO₃)=2.548 min.

### Step 2: Tridecyl L-alaninate

To a mixture of tridecyl (tert-butoxycarbonyl)-L-alaninate (9 g, 24.22 mmol) in DCM (90 mL) was added TFA (16.01 mL, 208 mmol) at 0° C. The mixture was stirred at 25° C. for 2 h. LCMS showed the presence of product. The solution was concentrated and pH of the residue was adjusted to 8-9 with NaOH (1N). The mixture was extracted with DCM (100 mL×3). The combined organic phases were washed with brine, dried over Na₂SO₄ and concentrated. The residue was purified by silica gel chromatography (pet. ether:EtOAc=1:1.2) to afford tridecyl L-alaninate (6 g, 22.10 mmol, 91% yield) as white solid. LCMS (M+H)=272.4; Retention time=1.917 min.

### Step 3: Tridecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of tridecyl L-alaninate (3.05 g, 11.25 mmol), triethylamine (1.568 mL, 11.25 mmol) in DCM (50 mL) was added phenyl phosphorodichloridate (1.681 mL, 11.25 mmol) in DCM (1 mL) dropwise under an atmosphere of nitrogen at about 0° C., and the reaction mixture was stirred at 0° C. for 1 h. To a solution of (2R,3S,5R)-5-(6-amino-2-fluoro-9H-purin-9-yl)-2-ethynyl-2-(hydroxymethyl)tetrahydrofuran-3-ol (1.5 g, 5.11 mmol) in THF (20 mL)/pyridine (10 mL) was added tert-butylmagnesium chloride (12.79 mL, 12.79 mmol) dropwise under an atmosphere of nitrogen at 0° C. Then, the mixture was stirred at room temperature for 30 min. The first prepared mixture was added dropwise to the second prepared mixture at 0° C. and stirred at 0-5° C. for 2.5 h. LCMS indicated the presence of product. Sat. NH₄Cl was added to the reaction. The mixture was filtered and concentrated to afford yellow solid. Then, the solid was dissolved in EtOAc, filtered and concentrated to afford the crude product. The residue was purified by reverse phase chromatography (SepaFlash® C18 column, 0-100% CH₃CN/10 mM NH₄HCO₃ H₂O) to obtain tridecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (300 mg, 0.396 mmol, 7.74% yield) as yellow solid. LCMS (M+H)=703.3, Retention time (0.1% TFA)=2.100 min. The diastereomers were separated by Prep-SFC (Instrument: SFC-200 (Thar, Waters), Column: OX 20×250 mm, 10 μm (Daicel), Column temperature: 35° C., Mobile phase:CO₂/EtOH (0.2% ammonia/methanol)=70/30, Flow rate: 120 g/min, Back pressure: 100 bar, Detection wavelength: 214 nm, Cycle time: 6.8 min, Sample solution: 680 mg dissolved in 60 ml methanol, Injection volume: 1.5 mL) to give first eluting isomer (Example 27A, RT1: 1.86 min, 155 mg, 0.218 mmol, 22.50% yield) as white solid; LCMS: LCMS (M+H)=703.3, Retention time (0.1% TFA): 2.092 min; HPLC: Retention time (0.1% NH₄HCO₃): 10.443 min; ¹H NMR (400 MHz, CDCl₃) δ 7.85 (s, 1H), 7.31 (t, J=7.9 Hz, 2H), 7.21 (d, J=8.5 Hz, 2H), 7.16 (t, J=7.4 Hz, 1H), 6.35 (t, J=5.8 Hz, 1H), 6.05 (s, 2H), 4.75 (t, J=7.5 Hz, 1H), 4.39 (d, J=8.6 Hz, 2H), 4.13-3.98 (m, 3H), 3.76 (t, J=10.5 Hz, 1H), 3.66 (s, 1H), 2.76 (s, 1H), 2.71-2.66 (m, 2H), 1.63-1.55 (m, 2H), 1.35 (d, J=7.1 Hz, 3H), 1.25 (s, 20H), 0.88 (t, J=6.8 Hz, 3H) and second eluting isomer (Example 27B, RT2: 2.83 min, 210 mg, 0.295 mmol, 30.5% yield) as white solid; LCMS (M+H)=703.2, Retention time (0.1% TFA): 2.107 min; HPLC: Retention time (0.1% NH₄HCO₃): 10.498 min; ¹H NMR (400 MHz, CDCl₃) δ 7.96 (s, 1H), 7.28 (t, J=8.0 Hz, 2H), 7.18 (d, J=8.2 Hz, 2H), 7.14 (t, J=7.3 Hz, 1H), 6.40-6.15 (m, 3H), 4.86 (t, J=7.6 Hz, 1H), 4.44-4.30 (m, 2H), 4.15-3.91 (m, 5H), 2.81-2.68 (m, 3H), 1.62-1.53 (m, 2H), 1.37 (d, J=6.7 Hz, 3H), 1.23 (s, 20H), 0.87 (t, J=6.8 Hz, 3H).

### Example 28

### Tetradecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

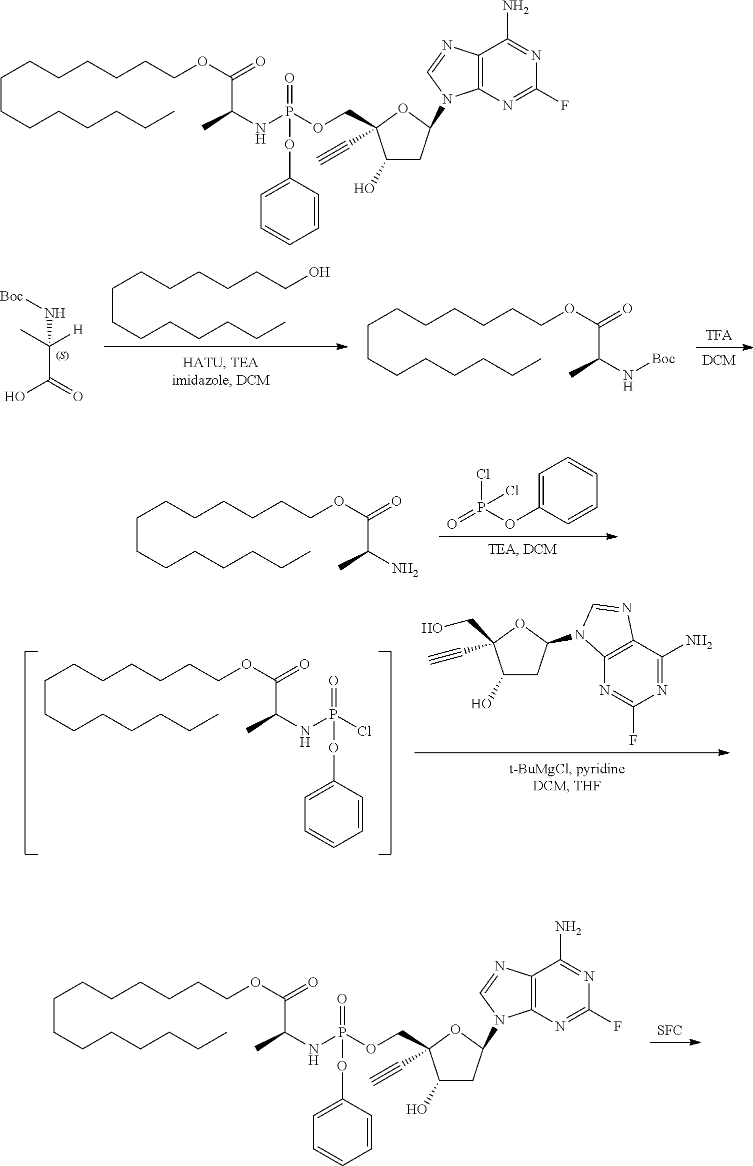

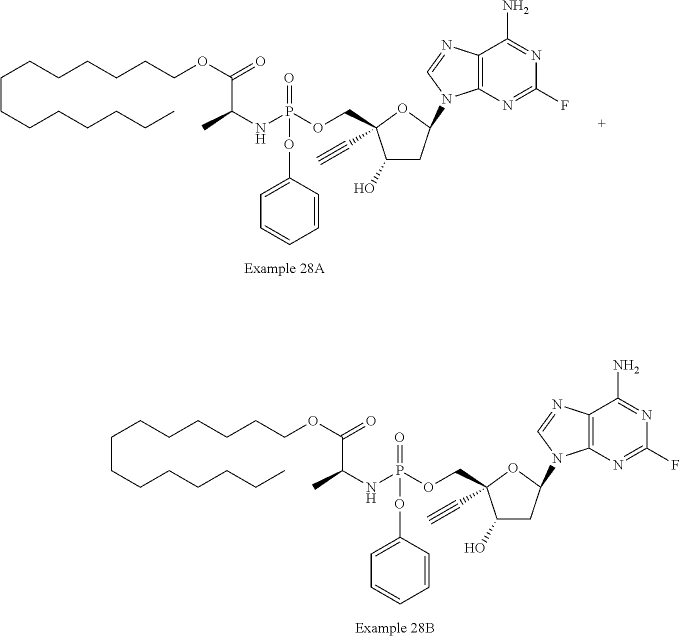 [SMILES: C#C[C@]1(COP(=O)(N[C@@H](C)C(=O)OCCCCCCCCCCCCCC)Oc2ccccc2)O[C@@H](n2cnc3c(N)nc(F)nc32)C[C@@H]1O ; C#C[C@]1(COP(=O)(N[C@@H](C)C(=O)OCCCCCCCCCCCCCC)Oc2ccccc2)O[C@@H](n2cnc3c(N)nc(F)nc32)C[C@@H]1O]

### Step 1: Tetradecyl (tert-butoxycarbonyl)-L-alaninate

To a solution of (tert-butoxycarbonyl)-L-alanine (12 g, 63.4 mmol), 2-(3H-[1,2,3]triazolo[4,5-b]pyridin-3-yl)-1,1,3,3-tetramethylisouronium hexafluorophosphate(V) (36.2 g, 95 mmol) and tetradecan-1-ol (16.32 g, 76 mmol) in DCM (200 mL) was added 1H-imidazole (12.95 g, 190 mmol) and N-ethyl-N-isopropylpropan-2-amine (24.59 g, 190 mmol). The resulting mixture was stirred at 25° C. overnight. TLC analysis showed the reaction was completed. The reaction was quenched with water (200 mL) and DCM (100 mL) then the organic layer was separated. The aqueous layer was extracted with EtOAc (2×200 mL), The combined organic layers were washed with brine (200 mL), dried with Na₂SO₄ and concentrated to give crude product. The residue was purified by combiflash (silica gel column 330 g, hexane:ethyl acetate=20:1). The appropriate fractions were combined and solvent removed in vacuo to give tetradecyl (tert-butoxycarbonyl)-L-alaninate (18 g, 42.0 mmol, 66.2% yield) as yellow solid. LCMS (M+Na)=408; Retention time (0.1% TFA)=3.22 min.

### Step 2: Tetradecyl L-alaninate

To a solution of tetradecyl (tert-butoxycarbonyl)-L-alaninate (30 g, 78 mmol) in DCM (150 mL) was added 2,2,2-trifluoroacetic acid (44.4 g, 389 mmol). The resulting mixture was stirred at room temperature for 16 h. LCMS analysis showed the reaction was completed. The reaction mixture was concentrated in vacuo to remove the volatiles and the residue was treated with 1 N NaHCO₃ (pH=5). The resulting cloudy, yellow solution was then partitioned with DCM, and the layers were separated. The aqueous layer was extracted with DCM, and the combined organic layers were dried over MgSO₄, filtered, and concentrated in vacuo to give tetradecyl L-alaninate (19 g, 66.6 mmol, 86% yield) which was used in the next step without purification. LCMS (M+H)=286; Retention time (0.1% TFA)=1.96 min.

### Step 3: Tetradecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold (ice-water bath) solution of tetradecyl L-alaninate (1217 mg, 4.26 mmol), triethylamine (0.594 mL, 4.26 mmol) in DCM (30 mL) was added phenyl phosphorodichloridate (899 mg, 4.26 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl) ethynyl-2-(hydroxymethyl)tetrahydrofuran-3-ol (500 mg, 1.705 mmol) in THF (20 mL)/pyridine (10 mL) was added tert-butylmagnesium chloride (4.26 mL, 4.26 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. The reaction mixture was quenched with water, partitioned between dichloromethane (100 mL) and water (100 mL). The organic phase was washed with water (100 mL), saturated brine (100 mL), dried over sodium sulphate and evaporated in vacuo to give the crude product. The residue was purified by silica gel column (25 g, MeOH:DCM=10:1) to give tetradecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (457 mg, 0.552 mmol, 32.4% yield). LCMS (M+H)=717.1; Retention time (0.1% TFA)=2.82 min. The diastereomers were separated by Prep-SFC (Instrument: SFC-80 (Thar, Waters); Column: AD 20×250 mm, 10 μm (Daicel); Column temperature: 40° C.; Mobile phase: CO₂/ETOH (1.0% ammonia/methanol)=45/55; Flow rate: 80 g/min; Back pressure: 100 bar; Detection wavelength: 214 nm; Cycle time: 7.7 min; Sample solution: 457 mg dissolved in 35 ml methanol; Injection volume: 3 mL) to give first eluting isomer (Example 28A, RT1: 1.69 min, 148.5 mg, 0.224 mmol, 100%, yield: 32.5%) as an off-white solid; LCMS (M+H)=717.1; Retention time (0.1% TFA)=2.81 min. ¹H NMR (400 MHz, DMSO) δ 8.24 (s, 1H), 7.84 (s, 2H), 7.32-7.27 (m, 2H), 7.13 (t, J=7.4 Hz, 3H), 6.28 (dd, J=7.6, 4.8 Hz, 1H), 5.98 (dd, J=13.4, 10.1 Hz, 1H), 5.81 (d, J=5.5 Hz, 1H), 4.65 (dd, J=12.7, 7.1 Hz, 1H), 4.22 (dd, J=10.8, 5.5 Hz, 1H), 4.10 (dd, J=10.9, 4.5 Hz, 1H), 3.97-3.89 (m, 2H), 3.73-3.65 (m, 2H), 2.81-2.73 (m, 1H), 2.48-2.42 (m, 1H), 1.50-1.43 (m, 2H), 1.20 (d, J=15.3 Hz, 22H), 1.13 (d, J=7.0 Hz, 3H), 0.85 (t, J=6.8 Hz, 3H). and second eluting isomer (Example 28B, RT2: 3.4 min, 154.8 mg, 0.231 mmol, 95.26%, yield: 32.3%) as an off-white solid; LCMS (M+H)=717.1; Retention time (0.1% TFA)=2.82 min. ¹H NMR (400 MHz, DMSO) δ 8.26 (s, 1H), 7.85 (s, 2H), 7.34-7.28 (m, 2H), 7.15 (dd, J=14.3, 7.5 Hz, 3H), 6.26 (dd, J=7.6, 4.9 Hz, 1H), 5.99 (dd, J=13.1, 10.0 Hz, 1H), 5.78 (d, J=5.4 Hz, 1H), 4.61 (dd, J=12.5, 7.2 Hz, 1H), 4.24 (dd, J=10.7, 5.9 Hz, 1H), 4.06 (dd, J=10.8, 4.7 Hz, 1H), 3.97-3.90 (m, 1H), 3.87-3.76 (m, 2H), 3.63 (s, 1H), 2.79-2.70 (m, 1H), 2.47-2.42 (m, 1H), 1.48-1.41 (m, 2H), 1.24-1.19 (m, 23H), 1.17 (s, 2H), 0.85 (t, J=6.8 Hz, 3H).

### Example 29

### Pentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

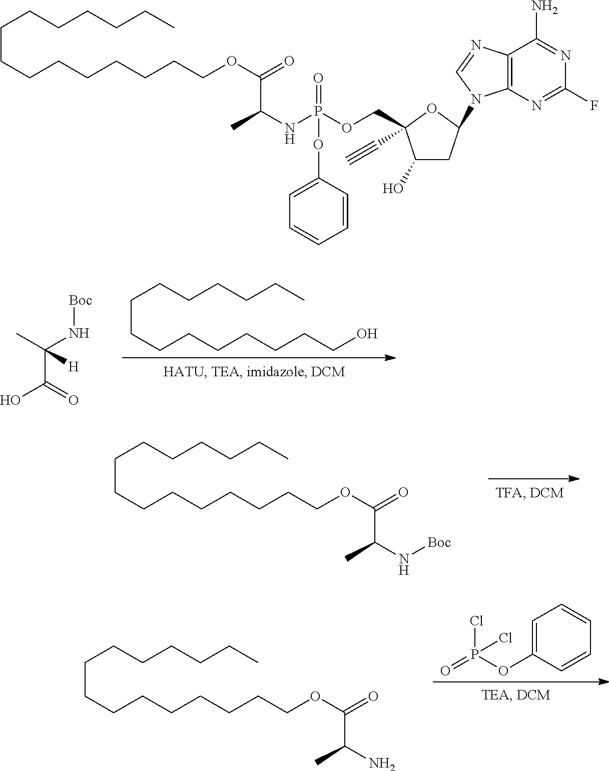 [SMILES: C#C[C@]1(COP(=O)(N[C@@H](C)C(=O)OCCCCCCCCCCCCCCC)Oc2ccccc2)O[C@@H](n2cnc3c(N)nc(F)nc32)C[C@@H]1O ; C[C@H](NC(=O)OC(C)(C)C)C(=O)O ; CCCCCCCCCCCCCCCO ; CCCCCCCCCCCCCCCOC(=O)[C@H](C)NC(=O)OC(C)(C)C ; CCCCCCCCCCCCCCCOC(=O)[C@H](C)N ; O=P(Cl)(Cl)Oc1ccccc1]

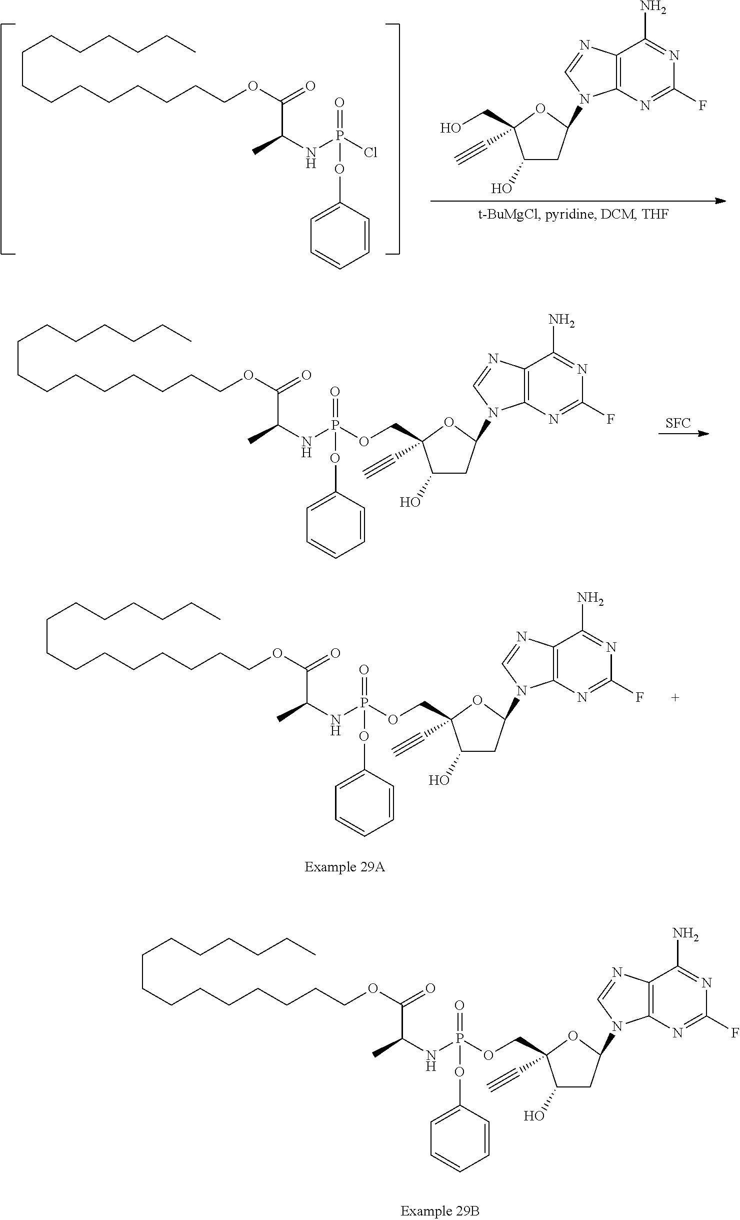

### Step 1: Pentadecyl (tert-butoxycarbonyl)-L-alaninate

To a mixture of pentadecan-1-ol (1.371 g, 6.00 mmol), tert-butoxycarbonyl)-L-alanine (1.135 g, 6 mmol), and HATU (3.42 g, 9 mmol) in DCM (20 mL) was added 1H-imidazole (1.225 g, 18.00 mmol) and DIPEA (3.14 mL, 18.00 mmol). The resulting mixture was stirred at 25° C. overnight. LCMS analysis showed the reaction was completed. Water (20 mL) was added and the mixture was extracted with DCM (20 mL×3). The combined organic layers were washed with water (20 mL×2), dried over Na₂SO₄, filtered and concentrated to afford pentadecyl (tert-butoxycarbonyl)-L-alaninate (2.4 g, 6.01 mmol, 100% yield) as colorless oil which was used in the next step without purification. ¹H NMR (400 MHz, CDCl₃) δ: 4.34-4.30 (m, 1H), 4.17-4.10 (m, 2H), 1.68-1.61 (m, 2H), 1.46 (s, 9H), 1.40 (d, J=7.2 Hz, 3H), 1.31-1.27 (m, 24H), 0.89 (t, J=6.8 Hz, 3H).

### Step 2: Pentadecyl L-alaninate

To a solution of pentadecyl (tert-butoxycarbonyl)-L-alaninate (2.398 g, 6 mmol) in DCM (6 mL) was added 2,2,2-trifluoroacetic acid (3 mL, 38.9 mmol). The resulting mixture was stirred at 25° C. for 3 h. LCMS showed the reaction was completed. The reaction was neutralized with NaHCO₃ (30 mL) and the aqueous layer was extracted with DCM (3×50 mL). The combined organic layers were washed with brine (40 mL), dried over Na₂SO₄, filtered and concentrated. The residue was purified by reverse phase chromatography (SepaFlash® C18 column, 0-100% CH₃CN/10 mM NH₄CO₃ H₂O) to give pentadecyl L-alaninate (1.62 g, 5.41 mmol, 90% yield) as colorless oil. LCMS (M+H)=300.1; Retention time (0.1% TFA)=2.04 min.

### Step 3: Pentadecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-alaninate

To a cold (ice-water bath) mixture of pentadecyl L-alaninate (2.043 g, 6.82 mmol), triethylamine (1.188 mL, 8.52 mmol) in DCM (42 mL) was added phenyl phosphorodichloridate (1439 mg, 6.82 mmol) in DCM (1 mL) dropwise under an atmosphere of nitrogen, then the reaction mixture was stirred at the same temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (28 mL) and pyridine (14 mL) was added tert-butylmagnesium chloride (8.52 mL, 8.52 mmol) dropwise under an atmosphere of nitrogen, then the mixture was stirred at the same temperature for 30 min. The first prepared mixture was added dropwise to the second prepared mixture at 0° C. and stirred at 0° C. for 2 h. LCMS showed the reaction was completed. The reaction mixture was filtered and concentrated. The residue was diluted with EtOAc (120 mL) and washed with water (50 mL×2). The aqueous layer was extracted with EtOAc (50 mL). The combined organic layers were dried with Na₂SO₄, filtered and concentrated. The residue was purified by reverse phase chromatography (SepaFlash® C18 column, 0-100% CH₃CN/10 mM NH₄HCO₃ H₂O) to give pentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (280 mg, 0.383 mmol, 11.24% yield) as white solid. LCMS (M+H)=731.2; Retention time (0.05% TFA)=2.28 min. The diastereomers were separated by Prep-SFC (Instrument: SFC-80 (Thar, Waters), Column: AD 20×250 mm, 10 μm (Daicel), Column temperature: 40° C., Mobile phase: CO₂/EtOH (1.0% ammonia/methanol)=40/60, Flow rate: 80 g/min, Back pressure: 100 bar, Detection wavelength: 260 nm, Cycle time: 8.5 min) to give first eluting isomer (Example 29A, RT1: 1.26 min, 89 mg, 0.122 mmol, 100%, yield: 31.8%) as a white solid; LCMS (M+H)=731.4; Retention time (0.05% TFA)=2.27 min. HPLC Retention time (10 mM NH₄HCO₃)=8.57 min. ¹H NMR (400 MHz, CDCl₃) δ 7.82 (s, 1H), 7.32 (t, J=8.0 Hz, 2H), 7.21 (d, J=7.6 Hz, 2H), 7.16 (t, J=7.2 Hz, 1H), 6.35 (t, J=5.6 Hz, 1H), 6.04 (brs, 2H), 4.79-4.71 (m, 1H), 4.39 (d, J=8.4 Hz, 2H), 4.14-3.96 (m, 3H), 3.80-3.70 (m, 2H), 2.76 (s, 1H), 2.69 (t, J=6.8 Hz, 2H), 1.64-1.55 (m, 2H), 1.35 (d, J=7.2 Hz, 3H), 1.29-1.20 (m, 24H), 0.88 (t, J=6.8 Hz, 3H) and second eluting isomer (Example 29B, RT2: 2.39 min, 80 mg, 0.109 mmol, 99.73%, yield: 28.6%) as a white solid; LCMS (M+H)=731.2; Retention time (0.05% TFA)=2.29 min. HPLC Retention time (10 mM NH₄HCO₃)=8.64 min. ¹H NMR (400 MHz, CDCl₃) δ 7.98 (s, 1H), 7.28 (t, J=8.0 Hz, 2H), 7.18 (d, J=8.0 Hz, 2H), 7.13 (t, J=7.2 Hz, 1H), 6.69 (brs, 2H), 6.36-6.32 (m, 1H), 4.85 (t, J=7.2 Hz, 1H), 4.44-4.31 (m, 4H), 4.12-3.95 (m, 3H), 2.76-2.71 (m, 3H), 1.60-1.52 (m, 2H), 1.35 (d, J=6.8 Hz, 3H), 1.27-1.20 (m, 24H), 0.88 (t, J=6.8 Hz, 3H).

### Example 30

### Hexadecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-alaninate

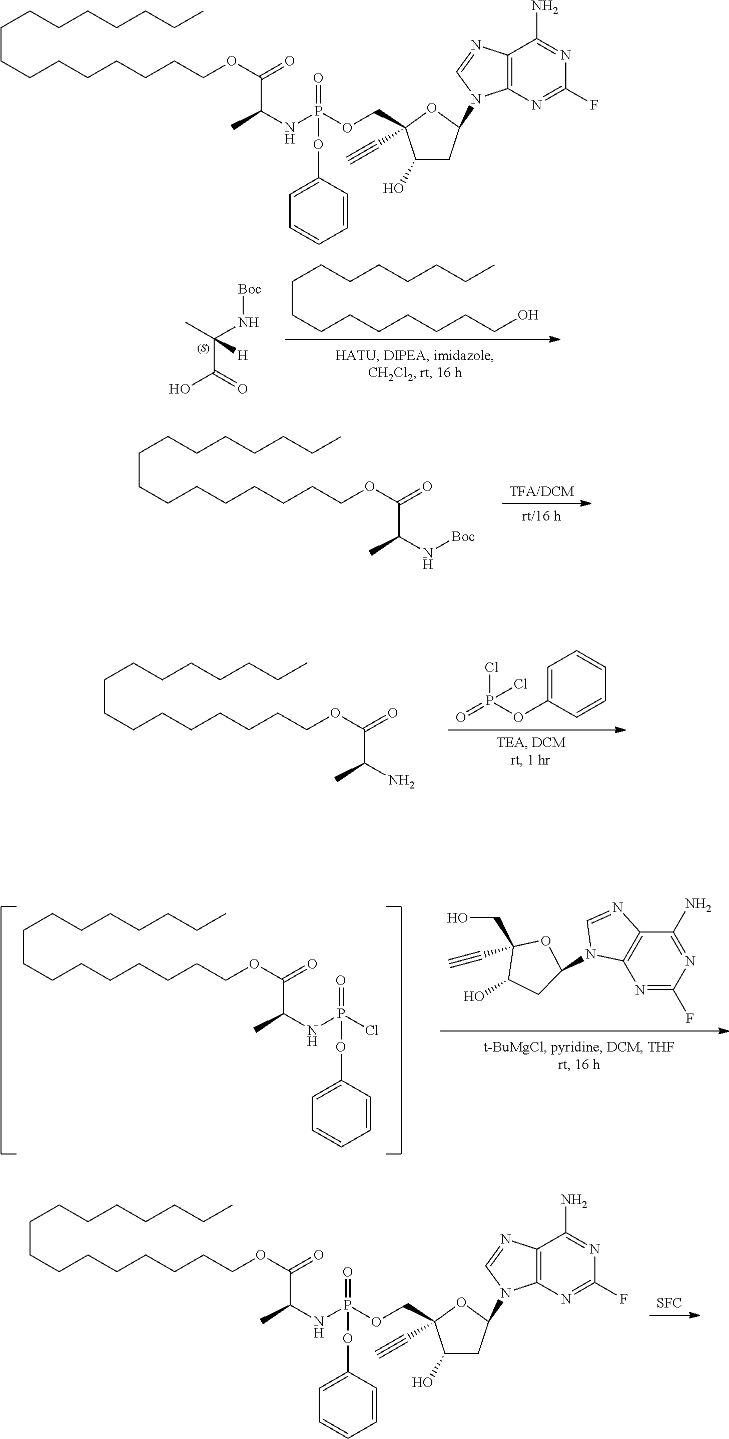

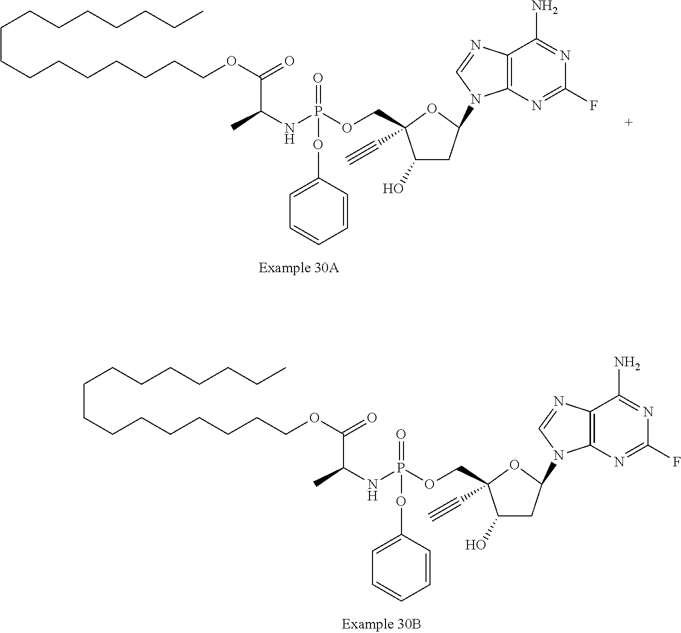 [SMILES: C#C[C@]1(COP(=O)(N[C@@H](C)C(=O)OCCCCCCCCCCCCCCCC)Oc2ccccc2)O[C@@H](n2cnc3c(N)nc(F)nc32)C[C@@H]1O ; C#C[C@]1(COP(=O)(N[C@@H](C)C(=O)OCCCCCCCCCCCCCCCC)Oc2ccccc2)O[C@@H](n2cnc3c(N)nc(F)nc32)C[C@@H]1O]

### Step 1: Hexadecyl (tert-butoxycarbonyl)-L-alaninate

To a solution of hexadecan-1-ol (1.409 g, 5.81 mmol), (tert-butoxycarbonyl)-L-alanine (1 g, 5.29 mmol) and HATU (3.01 g, 7.93 mmol) in DCM (10 mL) was added 1H-imidazole (1.079 g, 15.86 mmol) and DIPEA (2.77 mL, 15.86 mmol). The resulting mixture was stirred at 25° C. overnight. TLC (pet. ether:EtOAc=10:1, R.f=0.5) showed the reaction was completed. The reaction was quenched with water (20 mL) and DCM (10 mL). Then, the organic layer was separated and aqueous layer was extracted with DCM (2×10 mL). The combined organic layers were washed with brine (20 mL), dried with Na₂SO₄ and concentrated to give crude product. The residue was purified by combiflash (silica gel column 15 g, hexane:ethyl acetate=20:1). The appropriate fractions were combined and solvent removed in vacuum to give hexadecyl (tert-butoxycarbonyl)-L-alaninate (1.1 g, 2.66 mmol, 50.3% yield) as yellow solid.

### Step 2: Hexadecyl L-alaninate

To a solution of hexadecyl (tert-butoxycarbonyl)-L-alaninate (1 g, 2.418 mmol) in DCM (10 mL) was added TFA (0.931 mL, 12.09 mmol). The resulting mixture was stirred at 25° C. for 2 h. TLC (pet. ether:EtOAc=10:1, R.f=0.1) showed the reaction was completed. The reaction was quenched with water (20 mL) and DCM (10 mL), and the organic layer was separated. The aqueous layer was extracted with DCM (20 mL×2). The combined organic layers were washed with brine (20 mL), dried with Na₂SO₄ and concentrated to give crude product hexadecyl L-alaninate (688 mg, 2.194 mmol, 91% yield), which was used for the next step without purification.

### Step 3: hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold (ice-water bath) solution of hexadecyl L-alaninate (1.764 g, 5.63 mmol), triethylamine (0.784 mL, 5.63 mmol) in DCM (6 mL) was added phenyl phosphorodichloridate (1.187 g, 5.63 mmol) in DCM (1 mL) dropwise under an atmosphere of nitrogen. The reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1.1 g, 3.75 mmol) in THF (20 mL)/pyridine (10 mL) was added tert-butylmagnesium chloride (9.38 mL, 9.38 mmol) dropwise under an atmosphere of nitrogen, and the mixture was stirred at room temperature for 30 min. The first prepared mixture was added dropwise to the second prepared mixture at room temperature and stirred for 2 h. LCMS indicated presence of product. The reaction was quenched with water (100 mL) and EtOAc (100 mL), then the organic layer was separated. The aqueous layer was extracted by EtOAC (2×100 mL) and combined organic layers were washed with 1N HCL (50 mL), water (50 mL×2), followed by brine (50 mL), dried with Na2SO4, and concentrated to give crude product. The residue was purified by combiflash (silica gel column 80 g, DCM:MeOH=24:1). The appropriate fractions were combined and solvent was removed in vacuum to give hexadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (701 mg, 0.941 mmol, 25.09% yield) as yellow oil. LCMS (M+H)=745.3; Retention time (0.1% TFA)=2.38 min; The diastereomers were separated by Prep-SFC (Instrument: SFC-80 (Thar, Waters) Column: AD 20×250 mm, 10 μm (Daicel), Column temperature: 40° C. Mobile phase: CO₂/MeOH(1.0% ammonia/methanol)=40/60, Flow rate: 80 g/min; Back pressure: 100 bar; Detection wavelength: 214 nm; Cycle time: 9.6 min) to obtain first eluting isomer (Example 30A, RT1: 2.1 min, 180 mg, 0.242 mmol, 30.0% yield) as white solid; LCMS (M+H)=745.3; Retention time (10 mM NH₄HCO₃)=2.377 min; HPLC: Retention time (10 mM NH₄HCO₃)=13.121 min; ¹H NMR (400 MHz, CDCl₃) δ 7.86 (s, 1H), 7.32-7.26 (m, 2H), 7.19 (d, J=8.6 Hz, 2H), 7.13 (t, J=7.3 Hz, 1H), 6.35 (dd, J=6.6, 5.1 Hz, 2H), 6.33-6.27 (m, 1H), 4.76 (t, J=7.5 Hz, 1H), 4.42-4.38 (m, 2H), 4.10-4.03 (m, 2H), 4.02-3.97 (m, 1H), 3.89 (dd, J=27.2, 15.9 Hz, 2H), 2.75 (s, 1H), 2.71-2.65 (m, 2H), 1.92 (s, 2H), 1.34 (d, J=7.0 Hz, 3H), 1.25 (s, 26H), 0.87 (t, J=6.9 Hz, 3H) and second eluting isomer (Example 30B, RT2: 4.34 min, 250 mg, 0.336 mmol, 41.7% yield) as white solid. LCMS (M+H)=745.3; Retention time (10 mM NH₄HCO₃)=2. 383 min; HPLC: Retention time (10 mM NH₄HCO₃)=13.175 min; ¹H NMR (400 MHz, CDCl₃) δ 7.98 (s, 1H), 7.32-7.27 (m, 2H), 7.19 (d, J=8.6 Hz, 2H), 7.14 (t, J=7.3 Hz, 1H), 6.33 (dd, J=7.4, 4.1 Hz, 1H), 6.18 (s, 2H), 4.86 (t, J=7.6 Hz, 1H), 4.37 (ddd, J=20.2, 11.3, 8.4 Hz, 2H), 4.13-4.04 (m, 2H), 4.04-3.98 (m, 2H), 3.81 (s, 1H), 2.80-2.74 (m, 1H), 2.74-2.67 (m, 2H), 1.90 (s, 2H), 1.38 (d, J=6.2 Hz, 3H), 1.24 (d, J=4.6 Hz, 26H), 0.87 (t, J=6.8 Hz, 3H).

### Example 31

### Heptadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

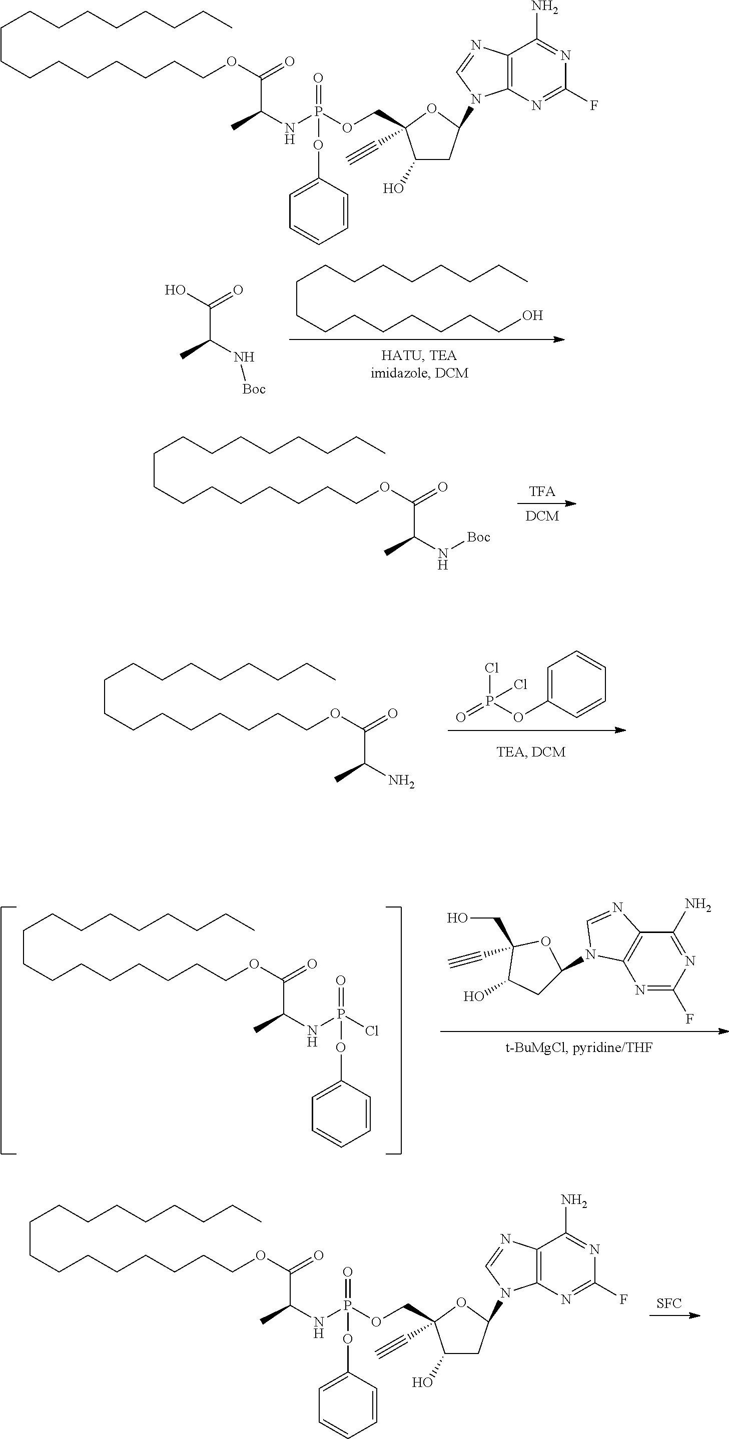

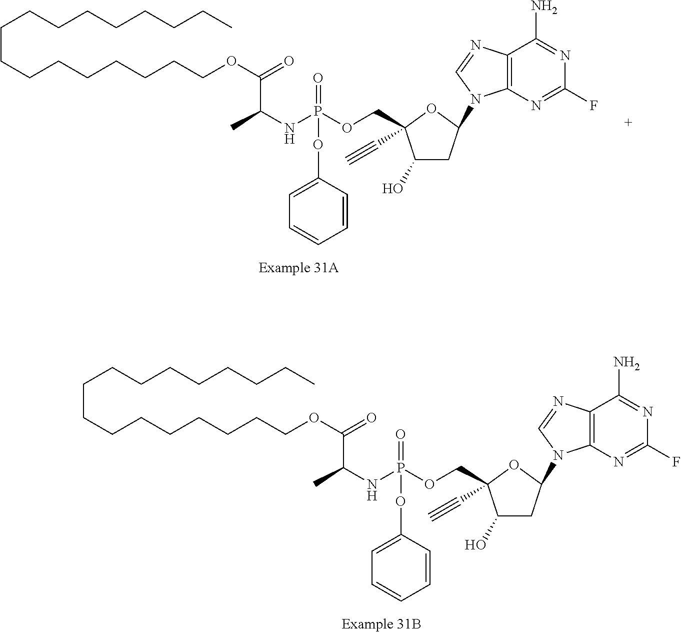 [SMILES: C#C[C@]1(COP(=O)(N[C@@H](C)C(=O)OCCCCCCCCCCCCCCCCC)Oc2ccccc2)O[C@@H](n2cnc3c(N)nc(F)nc32)C[C@@H]1O ; C#C[C@]1(COP(=O)(N[C@@H](C)C(=O)OCCCCCCCCCCCCCCCCC)Oc2ccccc2)O[C@@H](n2cnc3c(N)nc(F)nc32)C[C@@H]1O]

### Step 1: Heptadecyl (tert-butoxycarbonyl)-L-alaninate

A mixture of heptadecan-1-ol (12.5 g, 48.7 mmol), (tert-butoxycarbonyl)-L-alanine (9.22 g, 48.7 mmol), imidazole (9.95 g, 146 mmol), DIPEA (25.5 mL, 146 mmol) and HATU (27.8 g, 73.1 mmol) in DCM (120 mL) was stirred at 25° C. for 16 h. TLC showed the presence of new compound. The reaction was diluted with water (200 mL) and extracted with DCM (150 mL×3). The combined organic phases were washed with brine, dried over Na₂SO₄ and concentrated. The crude product was purified by flash chromatography (silica gel column 330 g, hexane:ethyl acetate=30:1) to give heptadecyl (tert-butoxycarbonyl)-L-alaninate (16.58 g, 38.8 mmol, 80% yield) as a colorless liquid. LCMS (M+H)=none; Retention time (0.1% TFA)=none. ¹H NMR (400 MHz, CDCl₃) δ 5.07 (d, J=7.4 Hz, 1H), 4.35-4.24 (m, 1H), 4.15-4.10 (m, 2H), 1.66-1.61 (m, 2H), 1.45 (s, 9H), 1.38 (d, J=7.2 Hz, 3H), 1.28 (d, J=17.9 Hz, 28H), 0.88 (t, J=6.8 Hz, 3H).

### Step 2: Heptadecyl L-alaninate

A mixture of heptadecyl (tert-butoxycarbonyl)-L-alaninate (16.58 g, 38.8 mmol) in TFA (30 mL, 389 mmol) and DCM (150 mL) was stirred at 25° C. overnight. LCMS showed the presence of product. The reaction mixture was concentrated, the pH of the resulting residue adjusted to 8-9 with NaOH (1N) and extracted with DCM (100 mL×3). The combined organic phases were washed with brine, dried over Na₂SO₄ and concentrated. The crude product was purified by flash chromatography (silica gel column 330 g, DCM:MeOH=10:1) to give heptadecyl L-alaninate (8.3 g, 25.3 mmol, 65.4% yield) as a white solid. ¹H NMR (400 MHz, d₆-DMSO) δ: 4.40-3.97 (m, 2H), 3.40-3.32 (m, 1H), 1.57-1.54 (m, 2H), 1.24-1.22 (m, 28H), 1.16-1.14 (m, 3H), 0.86-0.83 (m, 3H).

### Step 3: heptadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold (ice-water bath) solution of heptadecyl L-alaninate (1.675 g, 5.11 mmol) and TEA (0.951 mL, 6.82 mmol) in DCM (6.00 mL) was added phenyl phosphorodichloridate (1.079 g, 5.11 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (4 mL)/pyridine (2 mL) was added tert-butylmagnesium chloride (0.598 g, 5.11 mmol) dropwise under an atmosphere of nitrogen, and the mixture was stirred at room temperature for 30 min. The first prepared mixture was added dropwise to the second prepared mixture at room temperature and stirred for 16 h. LCMS indicated completion of reaction. The mixture was poured into water and extracted with EtOAc (50 mL). The organic phase was washed with brine (50 mL×3), dried over Na₂SO₄, filtered and concentrated. The crude product was purified by flash chromatography (silica gel column 40 g, DCM:MeOH=19:1) to give heptadecyl ((((2R,3S,5R)-5-(6-amino-2-fluoro-9H-purin-9-yl)-2-ethynyl-3-hydroxytetrahydrofuran yl)methoxy)(phenoxy)phosphoryl)-L-alaninate (500 mg, 0.626 mmol, 18.36% yield) as a white solid. LCMS (M+H)=759.1; Retention time (10 mM NH₄CO₃)=3.313 min. The diastereomers were separated by Prep-SFC (Instrument: SFC-80 (Thar, Waters); Column: Ad 20×250 mm, 5 μm (Daicel); Column temperature: 39.9° C.; Mobile phase: CO₂/MeOH(0.2% ammonia/methanol)=70/30; Flow rate: 120 g/min; Back pressure: 120 bar; Detection wavelength: 214 nm; Cycle time: 8 min; Sample solution: 500 mg dissolved in 50 ml methanol; Injection volume: 3 mL) to give first eluting isomer (Example 31A, RT1: 1.60 min, 124.3 mg, 0.158 mmol, 99.87, 24.05% yield) as an off-white solid; LCMS (M+H)=759.0; Retention time (10 mM NH₄HCO₃)=3.283 min. ¹H NMR (400 MHz, DMSO) δ 8.24 (s, 1H), 7.84 (s, 2H), 7.36-7.25 (m, 2H), 7.13 (t, J=7.4 Hz, 3H), 6.28 (dd, J=7.6, 4.7 Hz, 1H), 5.97 (dd, J=13.3, 10.1 Hz, 1H), 5.81 (d, J=5.5 Hz, 1H), 4.65 (dd, J=12.5, 7.0 Hz, 1H), 4.22 (dd, J=10.9, 5.5 Hz, 1H), 4.10 (dd, J=10.9, 4.6 Hz, 1H), 3.99-3.89 (m, 2H), 3.73-3.63 (m, 2H), 2.82-2.70 (m, 1H), 1.47 (s, 2H), 1.23 (s, 20H), 1.19 (s, 8H), 1.13 (d, J=7.1 Hz, 3H), 0.85 (t, J=6.8 Hz, 3H) and second eluting isomer (Example 31B, RT2: 2.37 min, 144.8 mg, 0.184 mmol, 91.54, 27.9% yield) as an off-white solid. LCMS (M+H)=759.0; Retention time (10 mM NH₄HCO₃)=3.312 min. ¹H NMR (400 MHz, DMSO) δ 8.26 (s, 1H), 7.85 (s, 2H), 7.35-7.27 (m, 2H), 7.20-7.10 (m, 4H), 6.27 (dd, J=7.6, 4.9 Hz, 1H), 5.98 (dd, J=13.1, 10.0 Hz, 1H), 5.79 (t, J=6.6 Hz, 1H), 4.61 (dd, J=12.7, 7.0 Hz, 1H), 4.24 (dd, J=10.7, 5.9 Hz, 1H), 4.06 (dd, J=10.8, 4.7 Hz, 1H), 3.93 (dd, J=11.9, 5.4 Hz, 1H), 3.87-3.75 (m, 2H), 3.63 (s, 1H), 2.74 (dd, J=11.8, 6.7 Hz, 1H), 1.44 (d, J=6.3 Hz, 2H), 1.23 (s, 20H), 1.18 (d, J=7.4 Hz, 11H), 0.84 (d, J=7.0 Hz, 3H).

### Example 32

### Octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

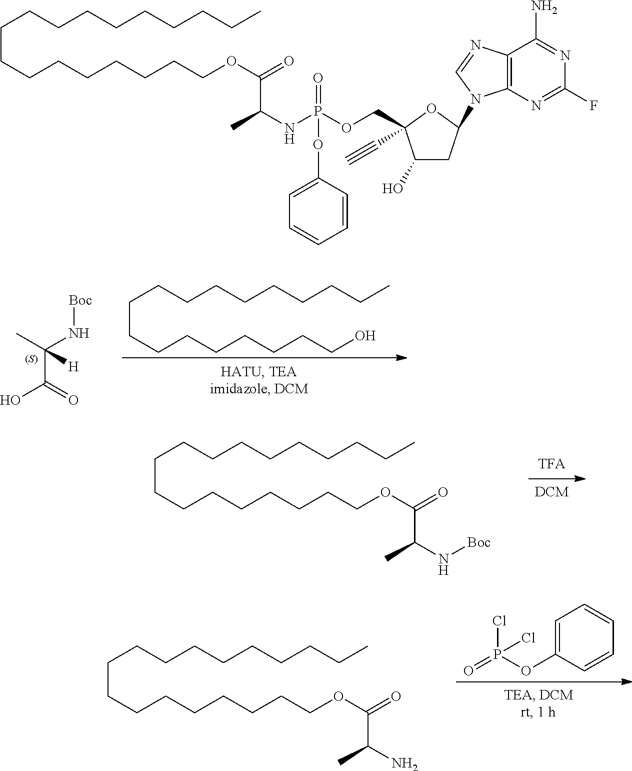

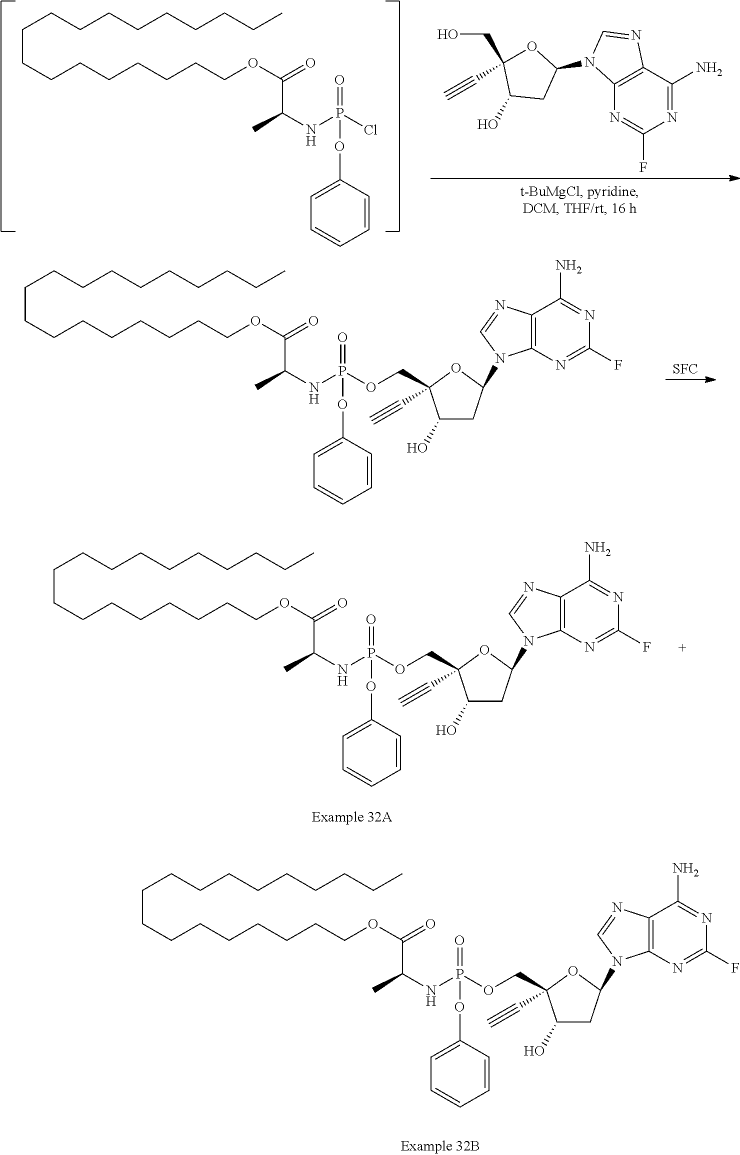

### Step 1: Octadecyl (tert-butoxycarbonyl)-L-alaninate

A mixture of octadecan-1-ol (24.92 g, 92 mmol), (tert-butoxycarbonyl)-L-alanine (16.6 g, 88 mmol), DIPEA (46.0 mL, 263 mmol), HATU (50.0 g, 132 mmol) and imidazole (17.92 g, 263 mmol) in DCM (200 mL) was stirred at 25° C. for 16 h. TLC (pet. ether:EtOAc=50:1, Rf=0.3) showed the presence of new product. Water (200 mL) was added, the mixture was extracted with DCM (150 mL×3). The combined organic phases were washed with brine, dried over Na₂SO₄ and concentrated. The residue was purified by silica gel chromatography (pet. ether:EtOAc=50:1) to afford octadecyl (tert-butoxycarbonyl)-L-alaninate (34.47 g, 78 mmol, 89% yield) as white solid. ¹H NMR (400 MHz, CDCl₃) δ 5.05 (d, J=6.3 Hz, 1H), 4.40-4.21 (m, 1H), 4.17-4.06 (m, 2H), 1.63 (t, J=7.1 Hz, 2H), 1.44 (s, 9H), 1.37 (d, J=7.2 Hz, 3H), 1.27 (d, J=18.1 Hz, 30H), 0.87 (t, J=6.8 Hz, 3H).

### Step 2: octadecyl L-alaninate

A mixture of octadecyl (tert-butoxycarbonyl)-L-alaninate (34.47 g, 78 mmol) in TFA (30 mL, 389 mmol) and DCM (150 mL) was stirred overnight at 25° C. LCMS showed the presence of new product. The reaction was concentrated and pH was of the residue was adjusted to 8-9 with NaOH (1N). The mixture was extracted with DCM (100 mL×3). The combined organic phases were washed with brine, dried over Na₂SO₄ and concentrated. The residue was purified by silica gel chromatography (DCM:MeOH=10:1) to afford octadecyl L-alaninate (26 g, 76 mmol, 98% yield) as white solid. LCMS (M+H)=342.2; Retention time (0.1% TFA)=2. 245 min; ¹H NMR (400 MHz, CDCl₃) δ: 4.12-4.08 (m, 2H), 3.55-3.54 (m, 1H), 1.68-1.61 (m, 4H), 1.34-1.33 (m, 5H), 1.33-1.22 (m, 28H), 0.89-0.86 (m, 3H).

### Step 3: Octadecyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-alaninate

To a cold (ice-water bath) solution of octadecyl L-alaninate (3.06 g, 8.95 mmol), triethylamine (1.248 mL, 8.95 mmol) in DCM (50 mL) was added phenyl phosphorodichloridate (1.888 g, 8.95 mmol) in DCM (1 mL) dropwise under an atmosphere of nitrogen. Then, the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1.05 g, 3.58 mmol) in THF (20 mL)/pyridine (10 mL) was added tert-butylmagnesium chloride (8.95 mL, 8.95 mmol) dropwise under an atmosphere of nitrogen, then the mixture was stirred at room temperature for 30 min. The first prepared mixture was added dropwise to the second prepared mixture at room temperature and stirred for 2 h. LCMS indicated the presence of new compound. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (30 mL×2). The combined organic phases were washed with brine, dried over Na₂SO₄, filtered and concentrated to afford the crude product. The residue was purified by reverse phase chromatography (SepaFlash® C18 column, 0-100% CH₃CN/10 mM NH₄HCO₃ H₂O) to obtain octadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (610 mg, 0.533 mmol, 14.90% yield) as white solid. LCMS (M+H)=773.4; Retention time (0.1% NH₄HCO₃): 3.50 min. The diastereomers were separated by Prep-SFC (Instrument: SFC-80 (Thar, Waters); Column: AD 20×250 mm, 10 μm (Daicel); Column temperature: 40° C.; Mobile phase: CO₂/MeOH(1.0% ammonia/methanol)=40/60; Flow rate: 80 g/min; Back pressure: 100 bar; Detection wavelength: 214 nm; Cycle time: 9.6 min)) to give first eluting isomer; (example 32A, RT1: 1.264 min, 250 mg, 0.323 mmol, 41.0% yield) as white solid; LCMS (M+H)=773.3; Retention time (0.1% TFA)=2.636 min; HPLC: Retention time (0.1% TFA)=14.191 min; ¹H NMR (400 MHz, CDCl₃) δ 7.89 (s, 1H), 7.31 (t, J=7.8 Hz, 2H), 7.23-7.18 (m, 2H), 7.16 (t, J=7.3 Hz, 1H), 6.35 (t, J=5.7 Hz, 1H), 6.14 (brs, 2H), 4.74 (t, J=7.5 Hz, 1H), 4.39 (d, J=8.4 Hz, 2H), 4.14-3.95 (m, 3H), 3.86-3.52 (m, 2H), 2.76 (s, 1H), 2.69 (dd, J=9.6, 5.1 Hz, 2H), 1.79 (s, 6H), 1.35 (d, J=7.1 Hz, 3H), 1.25 (s, 26H), 0.87 (t, J=6.8 Hz, 3H) and second eluting isomer (Example 32B, RT2: 1.724 min, 250 mg, 0.323 mmol, 41.0% yield) as white solid. LCMS (M+H)=773.4; Retention time (0.1% TFA)=2. 638 min; HPLC: Retention time (0.1% TFA)=14. 259 min; ¹H NMR (400 MHz, CDCl₃) δ 7.96 (s, 1H), 7.29 (t, J=8.4 Hz, 2H), 7.18 (d, J=8.6 Hz, 2H), 7.14 (t, J=7.4 Hz, 1H), 6.33 (dd, J=7.4, 4.1 Hz, 1H), 6.20 (brs, 1H), 4.86 (t, J=7.7 Hz, 1H), 4.46-4.29 (m, 2H), 4.19-3.83 (m, 5H), 2.87-2.63 (m, 3H), 1.89-1.74 (m, 4H), 1.38 (d, J=6.5 Hz, 3H), 1.25 (s, 28H), 0.88-0.87 (m, 3H).

### Example 33

### Tridecan-7-yl ((((2R,3S,5R)-5-(6-amino-2-fluoro-9H-purin-9-yl)-2-ethynyl hydroxytetrahydrofuran-2-yl)methoxy)(phenoxy)phosphoryl) alaninate

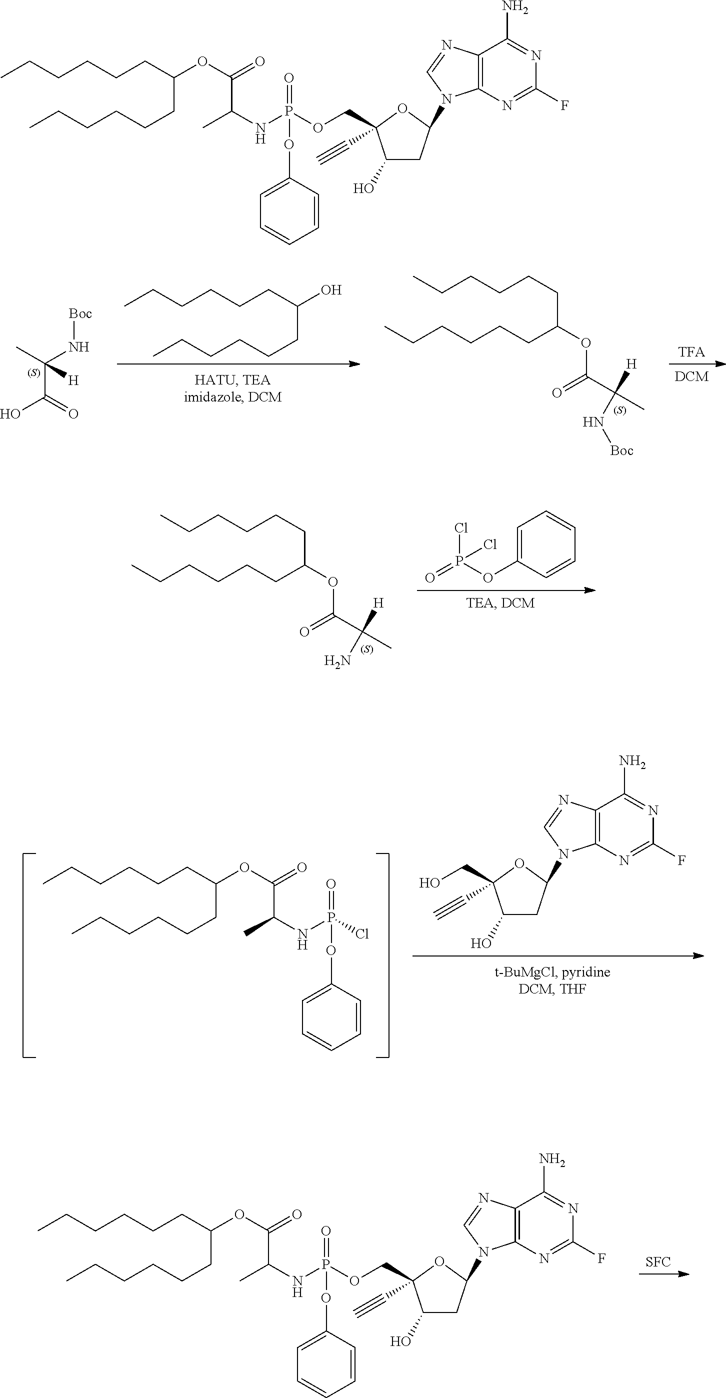

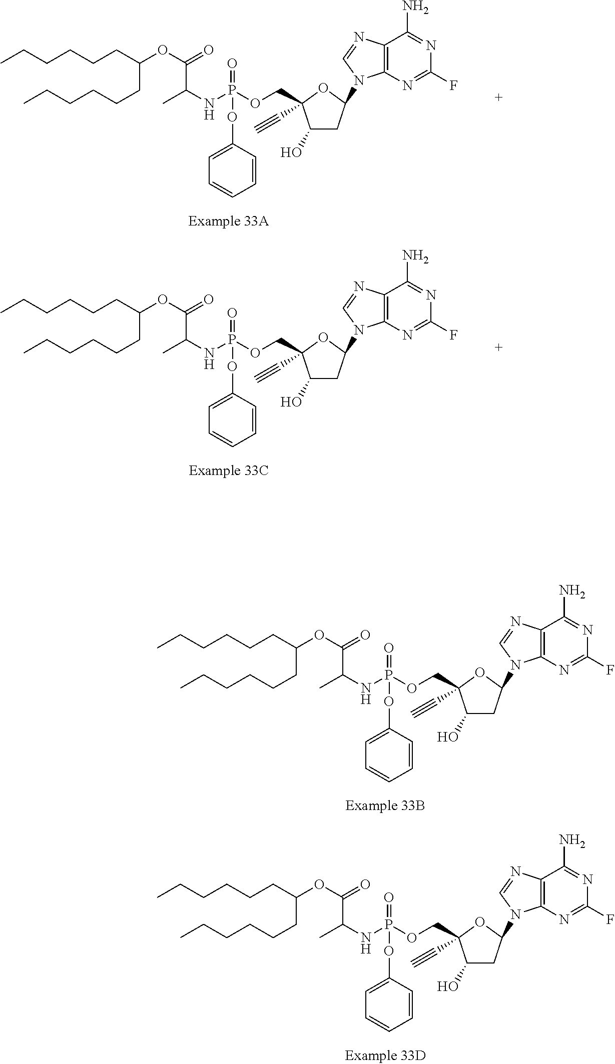 [SMILES: C#C[C@]1(COP(=O)(NC(C)C(=O)OC(CCCCCC)CCCCCC)Oc2ccccc2)O[C@@H](n2cnc3c(N)nc(F)nc32)C[C@@H]1O ; C#C[C@]1(COP(=O)(NC(C)C(=O)OC(CCCCCC)CCCCCC)Oc2ccccc2)O[C@@H](n2cnc3c(N)nc(F)nc32)C[C@@H]1O ; C#C[C@]1(COP(=O)(NC(C)C(=O)OC(CCCCCC)CCCCCC)Oc2ccccc2)O[C@@H](n2cnc3c(N)nc(F)nc32)C[C@@H]1O ; C#C[C@]1(COP(=O)(NC(C)C(=O)OC(CCCCCC)CCCCCC)Oc2ccccc2)O[C@@H](n2cnc3c(N)nc(F)nc32)C[C@@H]1O]

### Step 1: Tridecan-7-yl (tert-butoxycarbonyl)-L-alaninate

To a solution of (tert-butoxycarbonyl)-L-alanine (10.1 g, 53.4 mmol), tridecan-7-ol (11.23 g, 56.0 mmol), HATU (30.4 g, 80 mmol), imidazole (10.90 g, 160 mmol) and DIPEA (28.0 mL, 160 mmol) in DCM (200 mL) was stirred at 25° C. overnight. TLC showed starting material was consumed and a new compound was detected. Water (200 mL) was added and the reaction mixture was extracted with DCM (150 ml×3). The combined organic phases were washed with brine, dried over sodium sulfate and concentrated to afford the residue. The residue was purified by silica gel chromatography (pet. ether:EtOAc=100:1-20:1) to obtain tridecan-7-yl (tert-butoxycarbonyl)-L-alaninate (14.4 g, 36.8 mmol, 69.0 yield) as yellow solid.

### Step 2: Tridecan-7-yl L-alaninate

A solution of tridecan-7-yl (tert-butoxycarbonyl)-L-alaninate (14.4 g, 38.8 mmol) and TFA (25 mL, 324 mmol) in DCM (125 mL) was stirred at 25° C. overnight. TLC showed starting material was consumed and a new compound was detected. The reaction mixture was concentrated and the pH of the residue was adjusted to 8 with 1 N NaOH and the resulting mixture was extracted with DCM (100 ml×3). The combined organic phases were washed with brine, dried over sodium sulfate and concentrated to afford the residue. The residue was purified by silica gel chromatography (pet. ether:EtOAc=20:1-DCM:MeOH=20:1) to obtain tridecan-7-yl L-alaninate (10.3 g, 37.9 mmol, 98% yield) as yellow oil. ¹H NMR: (400 MHz, d6-DMSO): 4.93-4.87 (m, 1H), 3.84-3.79 (m, 1H), 1.55-1.53 (m, 4H), 1.50 (d, J=7.2 Hz, 3H), 1.22-1.26 (m, 16H), 0.88 (t, J=6.8 Hz, 6H).

### Step 3: Tridecan-7-yl ((((2R,3S,5R)-5-(6-amino-2-fluoro-9H-purin-9-yl)-2-ethynyl-3-hydroxytetrahydrofuran-2-yl) methoxy)(phenoxy)phosphoryl)-L-alaninate

To a cold (ice-water bath) solution of tridecan-7-yl L-alaninate (2314 mg, 8.52 mmol), triethylamine (1.188 mL, 8.52 mmol) in DCM (40 mL) was added phenyl phosphorodichloridate (1.274 mL, 8.52 mmol) in DCM (40 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1000 mg, 3.41 mmol) in THF (30 mL)/pyridine (15 mL) was added tert-butylmagnesium chloride (8.52 mL, 8.52 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. H₂O (20 mL) and EtOAc (30 mL) were added. The organic phase was separated and the aqueous layer was extracted with EtOAc (20 ml×2). The organic phases were combined, washed with water (20 mL×2), dried over Na₂SO₄ and concentrated. The crude purified by silica gel chromatography (DCM:MeOH=10:1) to give tridecan-7-yl ((((2R,3S,5R)-5-(6-amino-2-fluoro-9H-purin-9-yl)-2-ethynyl-3-hydroxytetrahydrofuran-2-yl) methoxy)(phenoxy)phosphoryl)-L-alaninate (320 mg, 0.450 mmol, 13.18% yield). LCMS (M+H)=703.3; Retention time (0.1% TFA)=2.04 min. The diastereomers were separated by Prep-SFC (Instrument: SFC-150 (Waters); Column: OX 20×250 mm, 10 μm (Daicel); Column temperature: 35° C.; Mobile phase: CO₂/MeOH(0.2% ammonia/methanol)=70/30; Flow rate: 100 g/min; Back pressure: 100 bar; Detection wavelength: 214 nm; Cycle time: 7 min; Sample solution: 700 mg dissolved in 40 ml methanol; Injection volume: 2 mL) to give first eluting isomer (Example 33A, RT1: 2.12 min, 100%) as an off-white solid; LCMS (M+H)=703.3; Retention time (0.1% TFA)=2.59 min. ¹H NMR (400 MHz, DMSO) δ 8.23 (s, 1H), 7.82 (brs, 2H), 7.29 (t, J=7.9 Hz, 2H), 7.13 (t, J=8.8 Hz, 3H), 6.28 (dd, J=7.6, 4.8 Hz, 1H), 5.94 (dd, J=13.4, 10.3 Hz, 1H), 5.80 (d, J=5.5 Hz, 1H), 4.75-4.60 (m, 2H), 4.21 (dd, J=10.8, 5.6 Hz, 1H), 4.08 (dd, J=10.9, 4.4 Hz, 1H), 3.73-3.63 (m, 2H), 2.80-2.72 (m, 1H), 2.48-2.41 (m, 1H), 1.47-1.37 (m, 4H), 1.22-1.07 (m, 19H), 0.80 (dt, J=17.3, 6.9 Hz, 6H); second eluting isomer (Example 33B, RT2: 2.43 min, 98.25%) as an off-white solid; LCMS (M+H)=703.3; Retention time (0.1% TFA)=2.60 min. ¹H NMR (400 MHz, DMSO) δ 8.19 (s, 1H), 7.84 (brs, 2H), 7.33-7.27 (m, 2H), 7.20-7.12 (m, 3H), 6.26 (dd, J=7.4, 5.1 Hz, 1H), 5.98 (dd, J=12.9, 10.3 Hz, 1H), 5.79 (d, J=5.5 Hz, 1H), 4.75-4.68 (m, 1H), 4.59 (dd, J=12.4, 6.8 Hz, 1H), 4.18 (dd, J=10.7, 5.1 Hz, 1H), 4.10 (dd, J=10.7, 5.1 Hz, 1H), 3.77-3.69 (m, 1H), 3.65 (s, 1H), 2.77-2.68 (m, 1H), 2.47-2.40 (m, 1H), 1.46-1.37 (m, 4H), 1.24-1.13 (m, 19H), 0.82 (td, J=6.8, 1.7 Hz, 6H); third eluting isomer (Example 33C, RT3: 2.89 min, 97.55%) as an off-white solid; LCMS (M+H)=703.3; Retention time (0.1% TFA)=2.61 min. ¹H NMR (400 MHz, DMSO) δ 8.19 (s, 1H), 7.84 (brs, 2H), 7.33-7.27 (m, 2H), 7.20-7.12 (m, 3H), 6.26 (dd, J=7.4, 5.1 Hz, 1H), 5.98 (dd, J=12.9, 10.3 Hz, 1H), 5.79 (d, J=5.5 Hz, 1H), 4.75-4.68 (m, 1H), 4.59 (dd, J=12.4, 6.8 Hz, 1H), 4.18 (dd, J=10.7, 5.1 Hz, 1H), 4.10 (dd, J=10.7, 5.1 Hz, 1H), 3.77-3.69 (m, 1H), 3.65 (s, 1H), 2.77-2.68 (m, 1H), 2.47-2.40 (m, 1H), 1.46-1.37 (m, 4H), 1.24-1.13 (m, 19H), 0.82 (td, J=6.8, 1.7 Hz, 6H). and fourth eluting isomer (Example 33D, RT4: 3.95 min, 99.54%) as an off-white solid; LCMS (M+H)=703.3; Retention time (0.1% TFA)=2.62 min. ¹H NMR (400 MHz, DMSO) δ 8.19 (s, 1H), 7.84 (brs, 2H), 7.33-7.27 (m, 2H), 7.20-7.12 (m, 3H), 6.26 (dd, J=7.4, 5.1 Hz, 1H), 5.98 (dd, J=12.9, 10.3 Hz, 1H), 5.79 (d, J=5.5 Hz, 1H), 4.75-4.68 (m, 1H), 4.59 (dd, J=12.4, 6.8 Hz, 1H), 4.18 (dd, J=10.7, 5.1 Hz, 1H), 4.10 (dd, J=10.7, 5.1 Hz, 1H), 3.77-3.69 (m, 1H), 3.65 (s, 1H), 2.77-2.68 (m, 1H), 2.47-2.40 (m, 1H), 1.46-1.37 (m, 4H), 1.24-1.13 (m, 19H), 0.82 (td, J=6.8, 1.7 Hz, 6H).

### Example 34

### Pentadecan-8-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

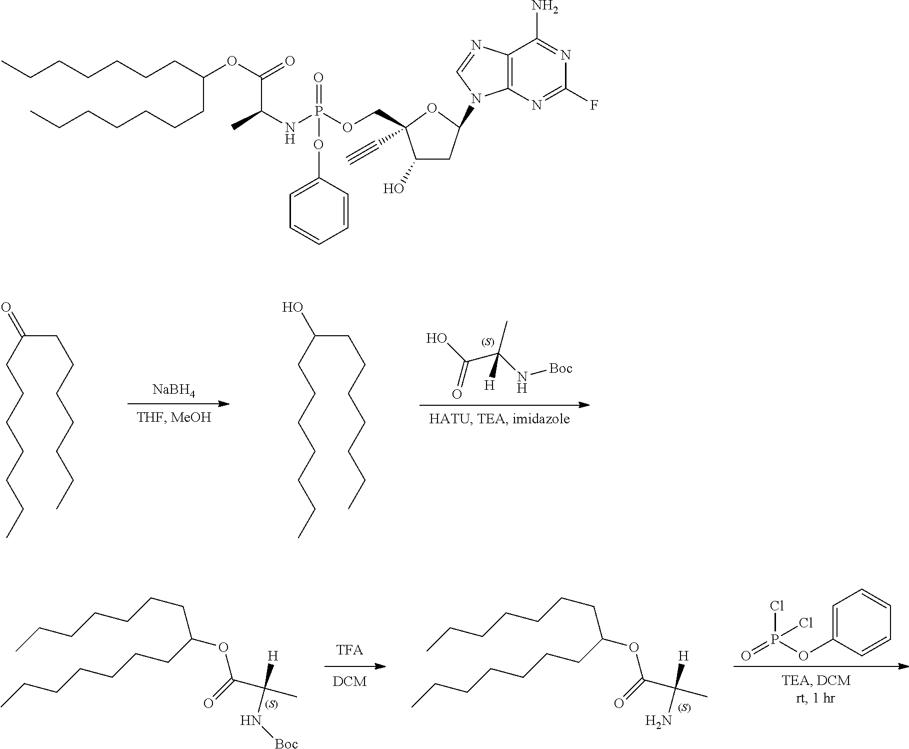

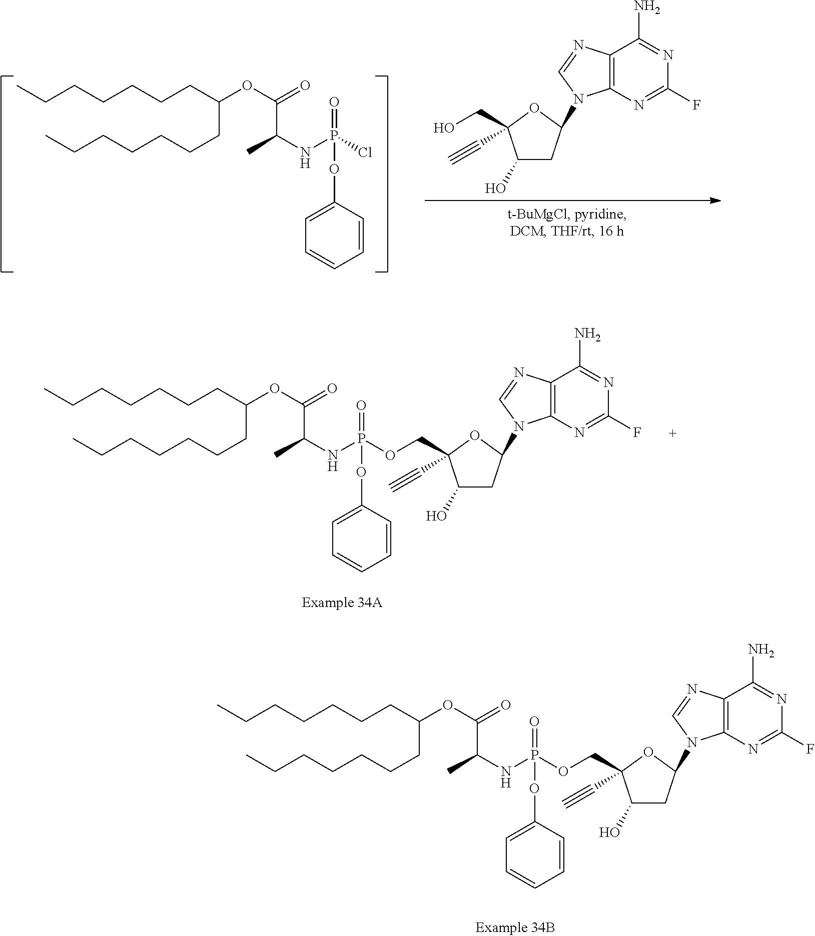

### Step 1: Pentadecan-8-ol

To a solution of pentadecan-8-one (10 g, 44.2 mmol) in THF (50 mL) and methanol (8.33 mL) was added NaBH₄ (2.506 g, 66.3 mmol) at 0° C. The reaction mixture was stirred at room temperature for 16 h. TLC showed the reaction was completed. The reaction mixture was quenched with NH₄Cl solution, concentrated to remove organic solvent and the aqueous mixture was extracted twice with EtOAc (80 mL). Then, the combined organic phases were washed with brine (80 mL), dried Na₂SO₄, filtered and concentrated to give crude product pentadecan-8-ol (9.5 g, 41.6 mmol, 94% yield) as white solid which was used in the next step.

### Step 2: pentadecan-8-yl (tert-butoxycarbonyl)-L-alaninate

To a solution of pentadecan-8-ol (9 g, 39.4 mmol), (tert-butoxycarbonyl)-L-alanine (7.46 g, 39.4 mmol) and HATU (22.47 g, 59.1 mmol) in DCM (300 mL) was added 1H-imidazole (8.05 g, 118 mmol) and DIPEA (20.65 mL, 118 mmol). The resulting mixture was stirred at 25° C. overnight. TLC (hexane:ethyl acetate=20:1, R.f=0.7) showed the reaction was completed. The reaction was quenched with water (100 mL) and DCM (100 mL). The organic layer was separated and aqueous layer was extracted with DCM (2×100 mL). Then combined organic layers were washed with brine (200 mL), dried with Na₂SO₄ and concentrated to give crude product. The residue was purified by combiflash (silica gel column 120 g, hexane:ethyl acetate=20:1). The appropriate fractions were combined and solvent removed in vacuo to give pentadecan-8-yl (tert-butoxycarbonyl)-L-alaninate (12 g, 30.0 mmol, 76% yield) as yellow oil.

### Step 3: Pentadecan-8-yl L-alaninate

To a solution of pentadecan-8-yl (tert-butoxycarbonyl)-L-alaninate (12 g, 30.0 mmol) in DCM (50 mL) was added trifluoroacetic acid (10.00 mL). The resulting mixture was stirred at 25° C. for 2 h. TLC (DCM:MeOH=20:1, R.f=0.4) showed the reaction was completed. The pH of the reaction mixture was adjusted with NaOH (aq., 1 mol/L) to ˜7 and extracted with DCM (2×100 mL). The combined organic layers were washed with brine (80 mL), dried with Na₂SO₄, concentrated to give crude product. The residue was purified by combiflash (silica gel column 120 g, DCM:MeOH=20:1). The appropriate fractions were combined and solvent was removed in vacuo to give pentadecan-8-yl L-alaninate (8 g, 26.7 mmol, 89% yield) as a yellow oil.

### Step 4: Pentadecan-8-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate

To a cold (ice-water bath) solution of pentadecan-8-yl L-alaninate (4.09 g, 13.64 mmol), triethylamine (1.901 mL, 13.64 mmol) in DCM (6 mL) was added phenyl phosphorodichloridate (2.88 g, 13.64 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydromethyl)tetrahydrofuran-3-ol (2 g, 6.82 mmol) in THF (4 mL)/pyridine (2 mL) was added tert-butylmagnesium chloride (17.05 mL, 17.05 mmol) dropwise under an atmosphere of nitrogen, and the mixture was stirred at room temperature for 30 min. The first prepared mixture was added dropwise to the second prepared mixture at room temperature and stirred at −20° C. for 60 h. LCMS showed the presence of new product. The cold (ice-water bath) reaction mixture was quenched with sat. NH₄Cl (20 mL), filtered and concentrated. The filtrate was extracted with EtOAc (30 mL×2). The combined organic phases were washed with sat. NaCl (20 mL), dried over Na₂SO₄ and concentrated. The residue was purified by reverse phase column chromatography (120 g, CH₃CN/10 mM NH₄CO₃ H₂O=1:3). The appropriate fractions were combined and solvent was removed in vacuo to give pentadecan-8-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate (350 mg, 0.464 mmol, 6.80% yield) as a white solid. LCMS: no mass, Retention time (0.1% TFA)=2.77 min. The diastereomers were separated by Prep-SFC (Instrument: SFC-150 (Waters); Column: OX 20×250 mm, 10 μm (Deicel): Column temperature: 35° C.; Mobile phase: CO₂/MeOH(0.2% ammonia/methanol)=60/40; Flow rate: 100 g/min; Back pressure: 100 bar; Detection wavelength: 214 nm; Cycle time: 5 min; Sample solution: 580 mg dissolved in 60 ml methanol; Injection volume: 2 mL) to afford first eluting isomer (Example 34A, RT1: 1.40 min, 180 mg, 0.242 mmol, 30.4 yield) as white solid; Retention time (0.1% TFA)=2.164 min, m/z=731.2 (M+H); HPLC: Retention time (0.1% NH₄HCO₃)=12.438 min; ¹H NMR (400 MHz, CDCl₃) δ 7.83 (s, 1H), 7.28 (d, J=8.3 Hz, 1H), 7.25-7.22 (m, 1H), 7.19 (d, J=8.0 Hz, 2H), 7.12 (t, J=7.2 Hz, 1H), 6.45 (s, 2H), 6.34 (dd, J=7.2, 4.2 Hz, 1H), 4.85 (p, J=6.2 Hz, 1H), 4.73 (t, J=7.6 Hz, 1H), 4.39 (d, J=8.2 Hz, 2H), 4.22-3.89 (m, 3H), 2.74 (s, 1H), 2.73-2.59 (m, 2H), 1.49 (d, J=4.5 Hz, 4H), 1.35 (d, J=6.7 Hz, 3H), 1.21 (d, J=6.4 Hz, 20H), 0.88-0.82 (m, 6H) and second eluting isomer (Example 34B, RT2: 2.42 min, 156 mg, 0.207 mmol, 26.0 yield) as white solid; Retention time (0.1% TFA)=2.189 min, m/z=731.3 (M+H); HPLC: Retention time (0.1% NH₄HCO₃)=12.549 min; ¹H NMR (400 MHz, CDCl₃) δ 7.98 (s, 1H), 7.24 (s, 1H), 7.17 (d, J=8.3 Hz, 2H), 7.11 (t, J=7.3 Hz, 1H), 6.82 (s, 2H), 6.33 (t, J=5.7 Hz, 1H), 4.88-4.79 (m, 2H), 4.46-4.24 (m, 4H), 4.06-3.95 (m, 1H), 2.76-2.66 (m, 3H), 1.46 (d, J=6.1 Hz, 4H), 1.36 (d, J=7.0 Hz, 3H), 1.20 (t, J=15.3 Hz, 20H), 0.83 (td, J=6.8, 3.3 Hz, 6H).

### Example 35

### Heptadecan-9-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

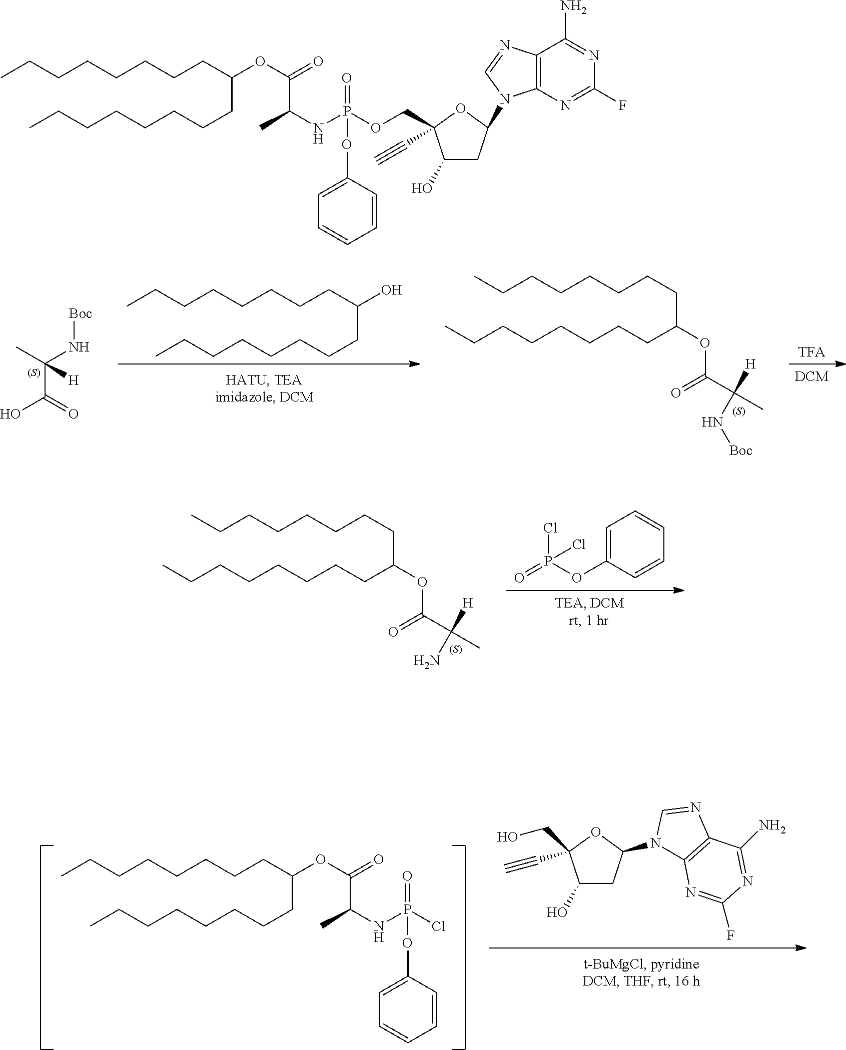

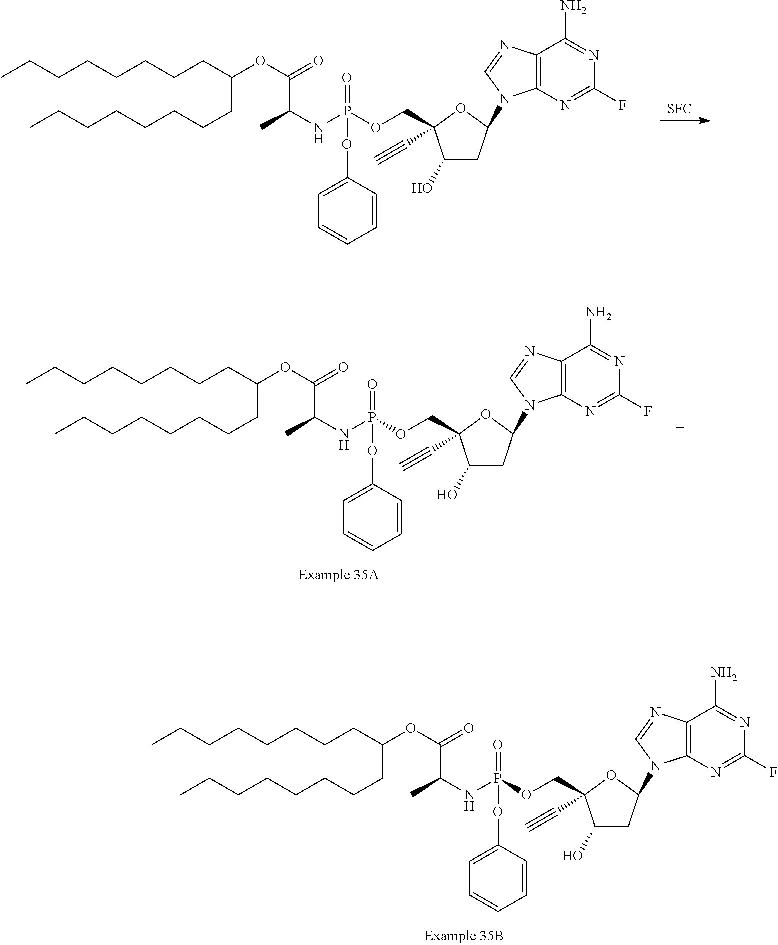

### Step 1: Heptadecan-9-yl (tert-butoxycarbonyl)-L-alaninate

A mixture of heptadecan-9-ol (12.5 g, 48.7 mmol), (tert-butoxycarbonyl)-L-alanine (9.22 g, 48.7 mmol), imidazole (9.95 g, 146 mmol), DIPEA (25.5 mL, 146 mmol) and HATU (27.8 g, 73.1 mmol) in DCM (120 mL) was stirred at 25° C. for 16 h. LCMS showed the presence of new product. Water (200 mL) was added, and the mixture was extracted with DCM (150 mL×3). The combined organic phases were washed with brine, dried over Na₂SO₄ and concentrated. The residue was purified by silica gel chromatography (DCM:MeOH=5:1) to afford heptadecan-9-yl (tert-butoxycarbonyl)-L-alaninate (18.3 g, 42.8 mmol, 88% yield) as white solid. ¹H NMR (400 MHz, CDCl₃) δ 5.09 (d, J=7.0 Hz, 1H), 4.94-4.85 (m, 1H), 4.36-4.20 (m, 1H), 1.52 (d, J=5.1 Hz, 4H), 1.44 (s, 9H), 1.37 (d, J=7.1 Hz, 3H), 1.25 (s, 24H), 0.87 (t, J=6.7 Hz, 6H).

### Step 2: Heptadecan-9-yl L-alaninate

A mixture of heptadecan-9-yl (tert-butoxycarbonyl)-L-alaninate (18.3 g, 42.8 mmol) in TFA (30 mL, 389 mmol) and DCM (150 mL) was stirred at 25° C. overnight. LCMS showed the presence of product. The reaction was concentrated and pH of the residue was adjusted to 8-9 with NaOH (1N), and the mixture was extracted with DCM (100 mL×3). The combined organic phases were washed with brine, dried over Na₂SO₄ and concentrated. The residue was purified by silica gel chromatography (DCM:MeOH=10:1) to afford heptadecan-9-yl L-alaninate (13.65 g, 41.7 mmol, 97% yield) as white solid. LCMS (M+H)=328.3; Retention time (0.1% TFA)=2.099 min. ¹H NMR (400 MHz, CDCl₃) δ: 4.89-4.87 (m, 1H), 3.53-3.51 (m, 1H), 1.62 (s, 2H), 1.53-1.52 (m, 4H), 1.33 (d, J=6.8 Hz, 3H), 1.29-1.25 (m, 24H), 0.89-0.86 (m, 6H).

### Step 3: Heptadecan-9-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a solution of heptadecan-9-yl L-alaninate (2.79 g, 8.52 mmol), triethylamine (1.188 mL, 8.52 mmol) in DCM (39.9 mL) was added phenyl phosphorodichloridate (1.274 mL, 8.52 mmol) in DCM (1 mL) dropwise under an atmosphere of nitrogen at about 5° C. Then, the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (20 mL)/pyridine (10 mL) was added tert-butylmagnesium chloride (8.52 mL, 8.52 mmol) dropwise under an atmosphere of nitrogen, then the mixture was stirred at room temperature for 30 min. The first prepared mixture was added dropwise to the second prepared mixture at room temperature and stirred for 2 h. LCMS indicated presence of product. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (30 mL×2). The combined organic phases were washed with brine, dried over Na₂SO₄, filtered and concentrated to afford the crude product. The residue was purified by reverse phase chromatography (SepaFlash® C18 column, 0-100% CH₃CN/10 mM NH₄HCO₃ H₂O) to obtain heptadecan-9-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (380 mg, 0.501 mmol, 14.68% yield). LCMS (M+H)=759.5; Retention time (0.1% TFA)=2.323, 2.351 min. The diastereomers were separated by pre-SFC (Instrument: SFC-80 (Thar, Waters) Column: OZ 20×250 mm, 10 μm (Daicel); Column temperature: 40° C.; Mobile phase: CO₂/MeOH(0.2% ammonia/methanol)=55/45; Flow rate: 80 g/min; Back pressure: 100 bar Detection wavelength: 260 nm Cycle time: 8 min)) to give first eluting isomer; (Example 35A RT1: 2.5 min, 181 mg, 0.229 mmol, 45.7% yield); LCMS (M+H)=759.3; Retention time (0.1% TFA)=2.294 min; HPLC: Retention time (0.1% TFA)=12.178 min; ¹H NMR (400 MHz, CDCl₃) δ 7.81 (s, 1H), 7.31 (t, J=7.9 Hz, 2H), 7.21 (d, J=8.4 Hz, 2H), 7.15 (t, J=7.2 Hz, 1H), 6.35 (dd, J=7.0, 4.7 Hz, 1H), 6.06 (s, 2H), 4.89-4.84 (m, 1H), 4.72 (t, J=7.5 Hz, 1H), 4.39 (d, J=8.9 Hz, 2H), 4.00 (dd, J=16.2, 8.0 Hz, 1H), 3.85-3.77 (m, 1H), 3.71 (s, 1H), 2.75 (s, 1H), 2.70-2.63 (m, 2H), 1.84 (s, 2H), 1.36 (d, J=7.0 Hz, 3H), 1.22 (s, 26H), 0.89-0.84 (m, 6H) and second eluting isomer (Example 35B, RT2: 5.9 min, 130 mg, 0.168 mmol, 33.5% yield) as white solid. LCMS (M+H)=759.4; Retention time (0.1% TFA)=2.352 min; HPLC: Retention time (0.1% NH₄HCO₃)=12.989 min; ¹H NMR (400 MHz, CDCl₃) δ 7.95 (s, 1H), 7.31-7.27 (m, 2H), 7.18 (d, J=8.1 Hz, 2H), 7.14 (t, J=7.4 Hz, 1H), 6.33 (dd, J=7.3, 4.2 Hz, 1H), 6.06 (s, 2H), 4.91-4.82 (m, 2H), 4.43-4.28 (m, 2H), 4.03 (dd, J=15.9, 8.9 Hz, 1H), 3.95-3.88 (m, 1H), 3.73 (s, 1H), 2.82-2.66 (m, 3H), 1.81 (s, 2H), 1.40 (d, J=6.9 Hz, 3H), 1.21 (d, J=13.5 Hz, 26H), 0.86 (td, J=6.8, 3.5 Hz, 6H).

### Example 36

Nonadecan-10-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

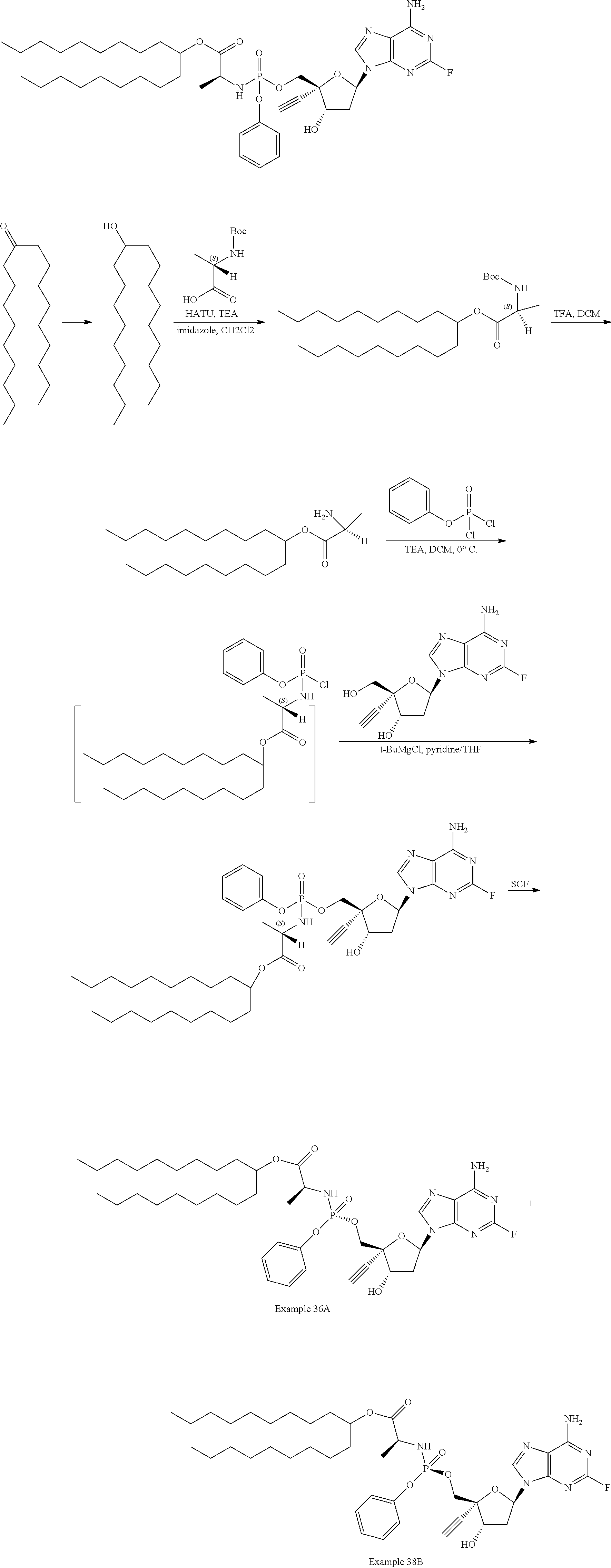

### Step-1: nonadecan-10-ol

To a solution of nonadecan-10-one (24 g, 85 mmol) in methanol (300 mL) and THF (1000 mL) was added NaBH4 (4.82 g, 127 mmol) at 0° C. The reaction mixture was stirred at 25° C. for 2 h and quenched with 200 (mL) sat. NH₄Cl by dropwise addition and concentrated to remove volatile organic solvents. The result mixture was diluted with H₂O (500 mL) and EtOAc (600 mL) and organic phase was separated. The aqueous layer was extracted with EtOAc (200 ml×2) and the combined organic phases were washed with water (200 mL×2), dried over Na₂SO₄ and concentrated to give crude nonadecan-10-ol (23 g, 81 mmol, 95% yield) as white solid.

### Step-2: Nonadecan-10-yl (tert-butoxycarbonyl)-L-alaninate

A mixture of (tert-butoxycarbonyl)-L-alanine (1.6 g, 8.46 mmol), nonadecan-10-ol (2.6 g, 9.14 mmol), imidazole (1.727 g, 25.4 mmol), DIPEA (4.43 mL, 25.4 mmol) and HATU (4.82 g, 12.68 mmol) in DCM (30 mL) was stirred at 25° C. for 16 h. TLC showed the presence of new product. Water (100 mL) was added, and the reaction mixture was extracted with DCM (150 mL×3). The combined organic phases were washed with brine, dried over Na₂SO₄, filtered and concentrated. The residue was purified by silica gel chromatography (pet. ether:EtOAc=20:1) to afford nonadecan-10-yl (tert-butoxycarbonyl)-L-alaninate (3 g, 1.975 mmol, 23.35% yield) as white solid.

### Step-3: Nonadecan-10-yl L-alaninate

To a solution of nonadecan-10-yl (tert-butoxycarbonyl)-L-alaninate (3 g, 6.58 mmol) in DCM (15 mL) was added TFA (5.07 mL, 65.8 mmol) at 25° C. and stirred for 4 h. TLC showed starting material was consumed completely. The reaction mixture was concentrated to dryness and the pH of residue was adjusted to 5-6 with sat. Na₂CO₃ solution, and then extracted with DCM (50 mL×3). The combined organic layers were washed with brine, dried over Na₂SO₄, filtered and concentrated. The residue was purified by silica gel chromatography to afford nonadecan-10-yl L-alaninate (1.8 g, 4.56 mmol, 69.2% yield). ¹H NMR (400 MHz, DMSO) δ 5.76 (s, 1H), 4.81-4.67 (m, 1H), 3.37 (dt, J=8.9, 4.5 Hz, 1H), 2.04 (d, J=39.2 Hz, 1H), 1.48 (s, 4H), 1.31-1.12 (m, 31H), 0.85 (t, J=6.8 Hz, 6H).

### Step-4: Nonadecan-10-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate

To a cold (ice-water bath) solution of nonadecan-10-yl alaninate (3032 mg, 8.52 mmol), triethylamine (1.188 mL, 8.52 mmol) in DCM (30 mL) was added phenyl phosphorodichloridate (1799 mg, 8.52 mmol) in DCM (1 mL) dropwise under an atmosphere of nitrogen, then the reaction mixture was stirred at room temperature for 1 h. To a cold solution of (2R,3S,5R)-5-(6-amino-2-fluoro-9H-purin-9-yl)-2-ethynyl-2-(hydroxymethyl)tetrahydrofuran-3-ol (1000 mg, 3.41 mmol) in THF (20 mL)/pyridine (10 mL) was added tert-butylmagnesium chloride (8.52 mL, 8.52 mmol) dropwise under an atmosphere of nitrogen at 0° C., and then the mixture was warmed and stirred at room temperature for 30 min. The first prepared mixture was added dropwise to the second prepared mixture at room temperature and stirred for 2 h. LCMS indicated the completion of reaction. The reaction mixture was filtered, concentrated, and then the residue was diluted with DCM and water. Then, the organic layer was washed with 0.5 N HCl three times and with brine. The organic phase was dried over Na₂SO₄, filtered and concentrated. The residue was purified by flash chromatography (DCM:MeOH=0-5%) to give nonadecan-10-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate (450 mg, 0.543 mmol, 15.93% yield). LCMS (M+H)=787.7; Retention time (0.1% TFA)=2.55 min. The diastereomers were separated by Prep-SFC (Instrument: SFC-80 (Waters), Column: OZ 20×250 mm, 10 μm (Daicel), Column temperature: 40° C.; Mobile phase: CO₂/MeOH(1.0% ammnia/methanol)=50/50; Flow rate: 80 g/min; Back pressure: 100 bar; Detection wavelength: 260 nm; Cycle time: 9 min) to afford first elutin isomer (Example 36A, RT1: 1.08 min, 180 mg, 0.224 mmol, 100.00%, yield: 44.1%) as an off-white solid; LCMS (M+H)=787.7; Retention time (0.1% TFA)=2.55 min. HPLC: Retention time (10 mM NH₄CO₃)=14.032 min. ¹H NMR (400 MHz, MeOD) δ 8.17 (s, 1H), 7.38-7.23 (m, 2H), 7.17 (dd, J=14.1, 7.9 Hz, 3H), 6.36 (dd, J=7.8, 4.1 Hz, 1H), 4.37 (dd, J=11.1, 6.2 Hz, 1H), 4.24 (dd, J=11.0, 4.5 Hz, 1H), 3.79-3.62 (m, 1H), 3.22 (s, 1H), 2.92-2.80 (m, 1H), 2.73-2.55 (m, 1H), 1.50 (s, 4H), 1.30-1.11 (m, 34H), 0.89 (td, J=6.9, 3.8 Hz, 6H) and second eluting isomer (Example 36B, RT2: 2.22 min, 110 mg, 0.137 mmol, 99.82%, yield: 27.1%) as an off-white solid; nonadecan-10-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate (110 mg, 0.137 mmol, 27.0% yield). LCMS (M+H)=787.7; Retention time (0.1% TFA)=2.59 min. HPLC: Retention time (10 mM NH₄HCO₃)=14.250 min. ¹H NMR (400 MHz, MeOD) δ 8.17 (s, 1H), 7.35-7.22 (m, 2H), 7.15 (dd, J=14.2, 7.5 Hz, 3H), 6.34 (dd, J=7.5, 4.1 Hz, 1H), 4.32 (ddd, J=32.4, 11.0, 6.0 Hz, 2H), 3.91 (dd, J=9.8, 7.0 Hz, 1H), 3.19 (s, 1H), 2.80 (dd, J=11.0, 6.6 Hz, 1H), 2.71-2.61 (m, 1H), 1.50 (s, 4H), 1.35-1.18 (m, 34H), 0.89 (td, J=6.8, 3.7 Hz, 6H).

### Example 37

### Henicosan-11-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

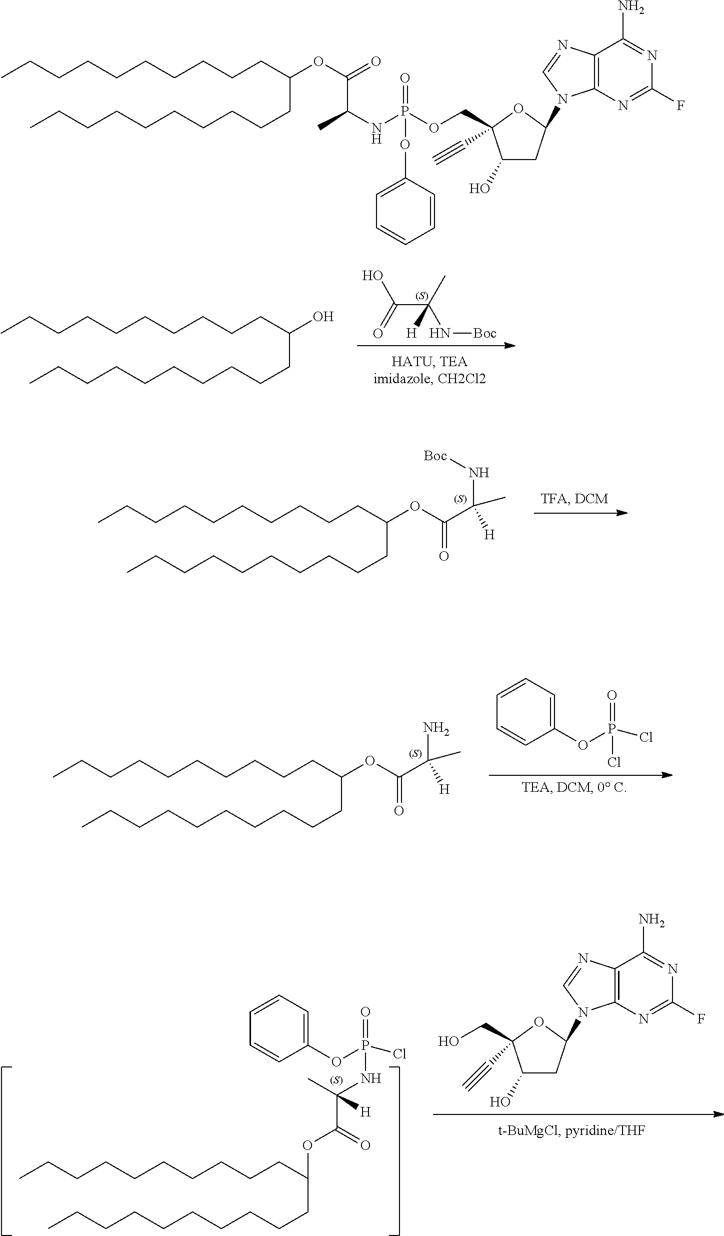

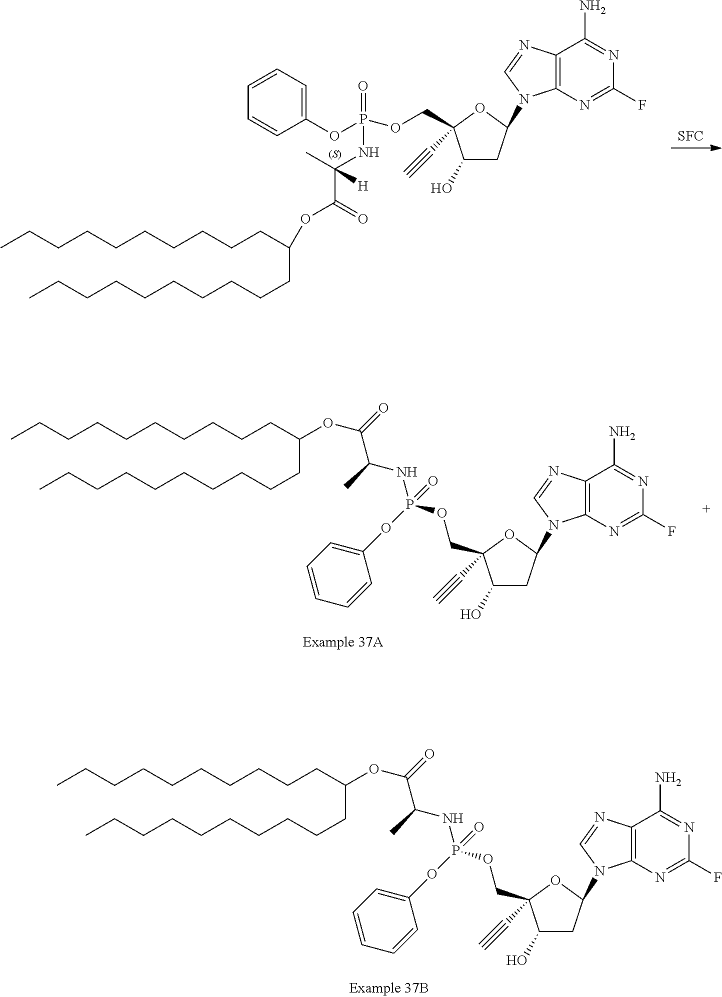

### Step 1: Henicosan-11-yl (tert-butoxycarbonyl)-L-alaninate

A mixture of (tert-butoxycarbonyl)-L-alanine (1.6 g, 8.46 mmol), henicosan-11-ol (10.6 g, 33.9 mmol), imidazole (1.727 g, 25.4 mmol), DIPEA (4.43 mL, 25.4 mmol) and HATU (4.82 g, 12.68 mmol) in DCM (100 mL) was stirred at 25° C. for 16 h. TLC showed the presence of new product. Water (100 mL) was added and the mixture was extracted with DCM (150 mL×3). The combined organic phases were washed with brine, dried over Na₂SO₄, filtered and concentrated. The residue was purified by silica gel chromatography (pet. ether:EtOAc=20:1) to afford henicosan-11-yl (tert-butoxycarbonyl)-L-alaninate (8 g, 4.96 mmol, 58.7% yield) as white solid.

### Step 2: Henicosan-11-yl L-alaninate

To a solution of henicosan-11-yl (tert-butoxycarbonyl)-L-alaninate (3 g, 6.20 mmol) in DCM (50 mL) was added TFA (4.78 mL, 62.0 mmol) at 25° C. and stirred for 4 h. TLC showed starting material was consumed completely. The reaction mixture was concentrated to dryness and pH of the residue was adjusted to 5-6 with sat. Na₂CO₃ solution, and extracted with DCM (100 mL×3). The combined organic layers were washed with brine, dried over Na₂SO₄, filtered and concentrated. The residue was purified by silica gel chromatography to afford henicosan-11-yl L-alaninate (2.4 g, 5.63 mmol, 91% yield). ¹H NMR (400 MHz, DMSO) δ 4.82-4.72 (m, 1H), 3.39 (dd, J=13.9, 6.9 Hz, 1H), 2.24 (s, 2H), 1.48 (s, 4H), 1.30-1.13 (m, 35H), 0.85 (t, J=6.8 Hz, 6H).

### Step 4: Henicosan-11-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate

To a cold (ice-water bath) solution of henicosan-11-yl alaninate (1308 mg, 3.41 mmol), triethylamine (0.475 mL, 3.41 mmol) in DCM (30 mL) was added phenyl phosphorodichloridate (719 mg, 3.41 mmol) in DCM (2 mL) dropwise under an atmosphere of nitrogen, then the reaction mixture was stirred at room temperature for 1 h. To a cold solution of (2R,3S,5R)-5-(6-amino-2-fluoro-9H-purin-9-yl)-2-ethynyl-2-(hydroxymethyl)tetrahydrofuran-3-ol (400 mg, 1.364 mmol) in THF (20 mL)/pyridine (8 mL) was added tert-butylmagnesium chloride (3.41 mL, 3.41 mmol) dropwise under an atmosphere of nitrogen at 0° C., and then the mixture was warmed and stirred at room temperature for 30 min. The first prepared mixture was added dropwise to the second prepared mixture at room temperature and stirred for 2 h. LCMS indicated the completion of reaction. The reaction mixture was filtered, concentrated, and then the residue was diluted with DCM and water. Then, the organic layer was washed with 0.5 N HCl three times and with brine. The organic phase was dried over Na₂SO₄, filtered and concentrated. The residue was purified by flash chromatography to afford henicosan-11-yl ((((2R,3S,5R)-5-(6-amino-2-fluoro-9H-purin-9-yl)-2-ethynyl-3-hydroxytetrahydrofuran-2-yl) methoxy) (phenoxy) phosphoryl)-L-alaninate (210 mg, 0.258 mmol, 18.89% yield). LCMS (M+H)=815.7; Retention time (0.1% TFA)=3.40 min. The diastereomers were separated by Prep-SFC (Instrument: SFC-80 (Waters), Column: OZ 20×250 mm, 10 μm (Daicel), Column temperature: 40° C.; Mobile phase: CO₂/MeOH(1.0% ammonia/methanol)=50/50; Flow rate: 80 g/min; Back pressure: 100 bar; Detection wavelength: 259 nm; Cycle time: 9 min) to afford first eluting isomer (Example 37A, RT1: 0.88 min, 130 mg, 0.152 mmol, 100.00%, yield: 30.9%) as an off-white solid; LCMS (M+H)=815.7; Retention time (0.1% TFA)=3.40 min. HPLC: Retention time (10 mM NH₄CO₃)=9.820 min. ¹H NMR (400 MHz, DMSO) δ 8.23 (s, 1H), 7.83 (s, 2H), 7.29 (t, J=7.9 Hz, 2H), 7.13 (t, J=7.5 Hz, 3H), 6.28 (dd, J=7.6, 4.9 Hz, 1H), 5.96 (dd, J=13.6, 10.3 Hz, 1H), 5.80 (d, J=5.5 Hz, 1H), 4.80-4.49 (m, 2H), 4.21 (dd, J=10.9, 5.6 Hz, 1H), 4.07 (dd, J=10.8, 4.3 Hz, 1H), 3.80-3.56 (m, 2H), 2.82-2.71 (m, 1H), 1.42 (s, 4H), 1.26-1.07 (m, 36H), 0.84 (td, J=6.9, 2.4 Hz, 6H) and second eluting isomer (Example 37B, RT2: 1.68 min, 121 mg, 0.158 mmol, 98.93%, yield: 32.2%) as an off-white solid; LCMS (M+H)=815.7; Retention time (0.1% TFA)=3.42 min. HPLC: Retention time (0.05% TFA)=16.378 min. ¹H NMR (400 MHz, DMSO) δ 8.26 (s, 1H), 7.84 (s, 2H), 7.39-7.21 (m, 2H), 7.21-7.10 (m, 3H), 6.27 (dd, J=7.4, 5.0 Hz, 1H), 5.97 (dd, J=12.7, 10.1 Hz, 1H), 5.78 (d, J=5.5 Hz, 1H), 4.76-4.52 (m, 2H), 4.25 (dd, J=10.8, 6.3 Hz, 1H), 4.14-4.06 (m, 1H), 3.81 (td, J=10.1, 7.2 Hz, 1H), 3.64 (s, 1H), 2.73 (dt, J=27.8, 11.4 Hz, 1H), 1.41 (s, 4H), 1.20 (d, J=7.2 Hz, 36H), 0.84 (dt, J=7.0, 3.3 Hz, 6H).

### Example 38

### Tricosan-12-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

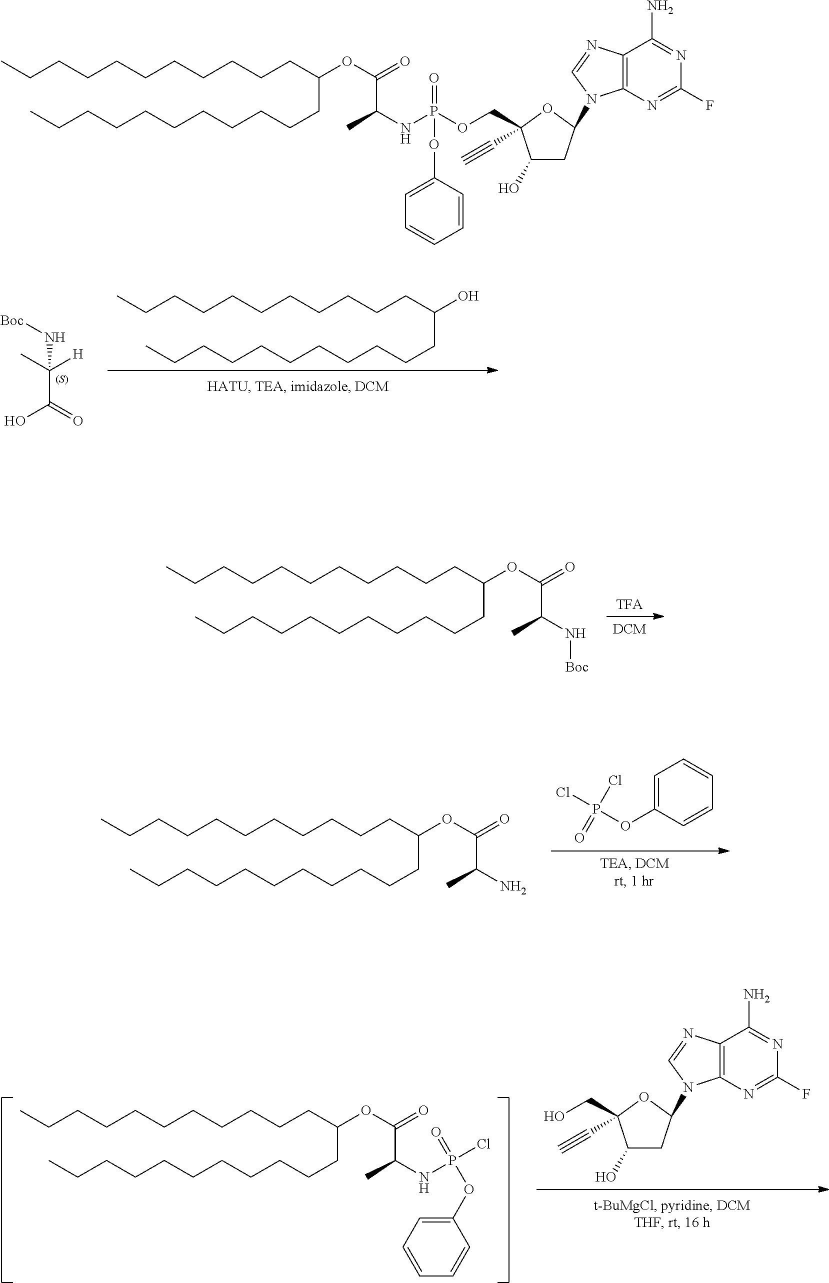

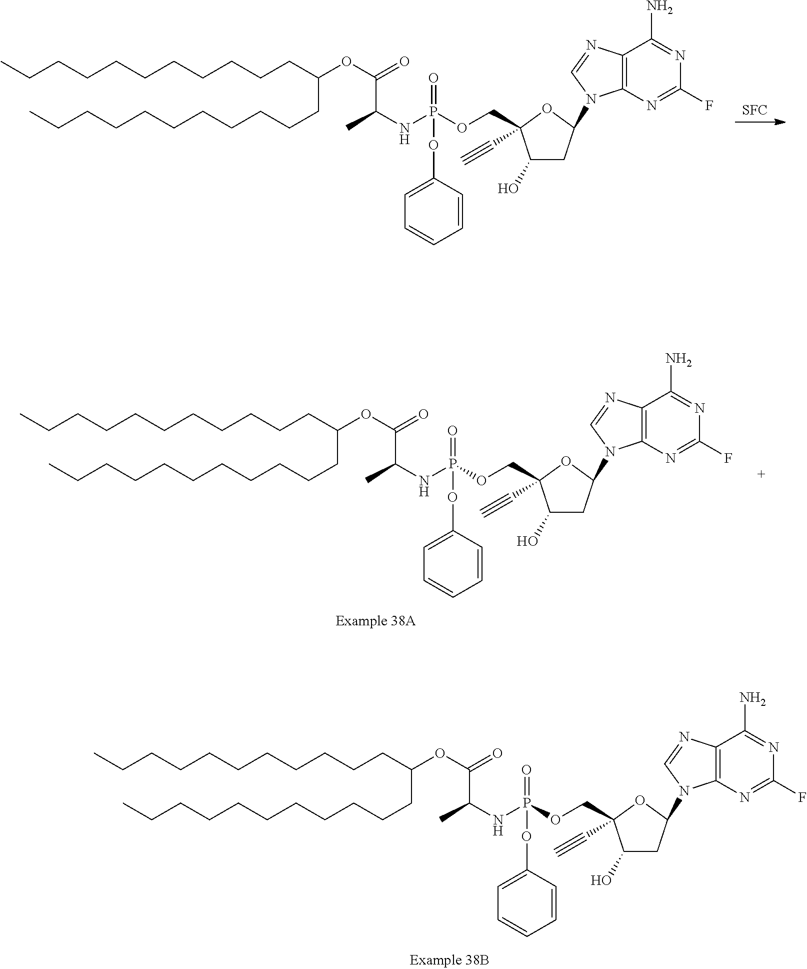

### Step 1: Tricosan-12-yl (tert-butoxycarbonyl)-L-alaninate

A mixture of (tert-butoxycarbonyl)-L-alanine (9.01 g, 47.62 mmol), tricosan-12-ol (17.03 g, 50.0 mmol), HATU (27.2 g, 71.4 mmol), imidazole (9.73 g, 143 mmol) and DIPEA (24.95 mL, 143 mmol) in DCM (200 mL) was stirred at 25° C. overnight. TLC showed presence of starting material and a new compound. Water was added and the mixture was extracted with DCM (150 ml×3). The combined organic phases were washed with brine, dried over Na₂SO₄ and concentrated to afford the residue. The residue was purified by silica gel chromatography (pet. ether:EtOAc=20:1) to obtain tricosan-12-yl (tert-butoxycarbonyl)-L-alaninate (16.7 g, 31.0 mmol, 65.1% yield) as yellow solid. ¹H NMR (400 MHz, d6-DMSO) δ: 7.26 (s, 1H), 4.78-4.76 (m, 1H), 3.95-3.91 (m, 1H), 1.53-1.46 (m, 4H), 1.27 (s, 9H), 1.23-1.11 (m, 39H), 0.83 (t, J=7.2 Hz, 6H).

### Step 2: Tricosan-11-yl L-alaninate

A mixture of tricosan-11-yl (tert-butoxycarbonyl)-L-alaninate (16.7 g, 32.6 mmol) and TFA (30 mL, 389 mmol) in DCM (150 mL) was stirred at 25° C. overnight. TLC showed starting material was consumed and a new compound was detected. The reaction mixture was concentrated and the pH of the residue was adjusted to 8 with 1 N NaOH and extracted with DCM (100 ml×3). The combined organic phases were washed with brine, dried over Na₂SO₄ and concentrated to afford residue. The residue was purified by silica gel chromatography (pet. ether:EtOAc=20:1-DCM:MeOH=10:1) to obtain tricosan-11-yl L-alaninate (7.4 g, 17.97 mmol, 55.1% yield) as yellow oil. ¹H NMR (400 MHz, CDCl₃) δ: 8.89 (t, J=6.4 Hz, 1H), 3.54 (q, J=6.8 Hz, 14.0 Hz, 1H), 2.06 (s, 2H), 1.50-1.53 (m, 4H), 1.35 (d, J=6.8 Hz, 3H), 1.30-1.32 (m, 36H), 0.88 (t, J=6.8 Hz, 6H).

### Step 3: Tricosan-12-yl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold solution of tricosan-12-yl L-alaninate (3.37 g, 8.18 mmol), triethylamine (1.141 mL, 8.18 mmol) in DCM (50 mL) was added phenyl phosphorodichloridate (1.223 mL, 8.18 mmol) in DCM (1 mL) dropwise under an atmosphere of nitrogen at about 5° C., then the reaction mixture was stirred at room temperature for 1 h. To a cold solution of (2R,3S,5R)-5-(6-amino-2-fluoro-9H-purin-9-yl)-2-ethynyl-2-(hydroxymethyl)tetrahydrofuran-3-ol (1.2 g, 4.09 mmol) in THF (20 mL)/pyridine (10 mL) was added tert-butylmagnesium chloride (10.23 mL, 10.23 mmol) dropwise under an atmosphere of nitrogen at 0° C. Then, the mixture was stirred at room temperature for 30 min. The first prepared mixture was added dropwise to the second prepared mixture at room temperature and stirred for 2 h. LCMS indicated presence of product. The mixture was filtered and concentrated to afford yellow solid. Then, the solid was dissolved in EtOAc, filtered and concentrated to afford the crude product. The residue was purified by reverse phase chromatography (SepaFlash® C18 column, 0-100% CH3CN/10 mM NH₄HCO₃ H₂O) to obtain tricosan-12-yl ((((2R,3S,5R) (6-amino-2-fluoro-9H-purin-9-yl)-2-ethynyl-3-hydroxytetrahydrofuran yl)methoxy)(phenoxy)phosphoryl)-L-alaninate (850 mg, 0.972 mmol, 23.76% yield) as yellow solid. LCMS: Retention time (0.1% TFA)=4.004, 4.097 min. The diasteromers were separated by prep-SFC (Instrument: SFC-80 (Thar, Waters); Column: OX 20×250 mm, 10 μm (Deicel): Column temperature: 35° C.; Mobile phase: CO₂/MeOH (0.2% ammonia/methanol)=55/45; Flow rate: 100 g/min; Back pressure: 100 bar; Detection wavelength: 214 nm; Cycle time: 4 min; Sample solution: 880 mg dissolved in 30 ml methanol and DCM) to afford first eluting isomer (Example 38A, RT1: 1.2 min, 296 mg, 0.338 mmol, 40.7% yield) as white solid; LCMS: Retention time (0.1% TFA)=3.907 min; HPLC: Retention time (0.1% TFA)=13.606 min; ¹H NMR (400 MHz, CDCl₃) δ 7.79 (s, 1H), 7.31 (t, J=7.9 Hz, 2H), 7.21 (d, J=7.7 Hz, 2H), 7.15 (t, J=7.3 Hz, 1H), 6.35 (dd, J=7.0, 4.7 Hz, 1H), 6.05 (s, 2H), 4.87 (d, J=6.2 Hz, 1H), 4.72 (t, J=7.5 Hz, 1H), 4.38 (d, J=8.9 Hz, 2H), 4.00 (dd, J=15.8, 8.1 Hz, 1H), 3.86-3.62 (m, 2H), 2.75 (s, 1H), 2.72-2.62 (m, 2H), 1.76 (s, 4H), 1.36 (d, J=7.0 Hz, 3H), 1.26-1.20 (m, 36H), 0.87 (t, J=6.8 Hz, 6H) and second eluting isomer (Example 38B, RT2: 2.56 min, 168 mg, 0.197 mmol, 23.73% yield) as white solid; LCMS (M+H)=843.4; Retention time (0.1% TFA)=3.983 min; HPLC: Retention time (0.1% TFA)=13.942 min; ¹H NMR (400 MHz, CDCl₃) δ 7.95 (s, 1H), 7.31-7.27 (m, 2H), 7.18 (d, J=8.6 Hz, 2H), 7.15 (t, J=7.3 Hz, 1H), 6.33 (dd, J=7.4, 4.2 Hz, 1H), 6.00 (s, 2H), 4.86 (dd, J=11.7, 5.8 Hz, 2H), 4.36 (ddd, J=20.6, 11.3, 8.7 Hz, 2H), 4.03 (dd, J=16.1, 9.0 Hz, 1H), 3.91-3.84 (m, 1H), 3.71 (s, 1H), 2.81-2.67 (m, 3H), 1.77 (s, 4H), 1.40 (d, J=7.0 Hz, 3H), 1.26-1.18 (d, J=14.5 Hz, 36H), 0.87 (t, J=6.9 Hz, 6H).

### Example 39

Hexadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

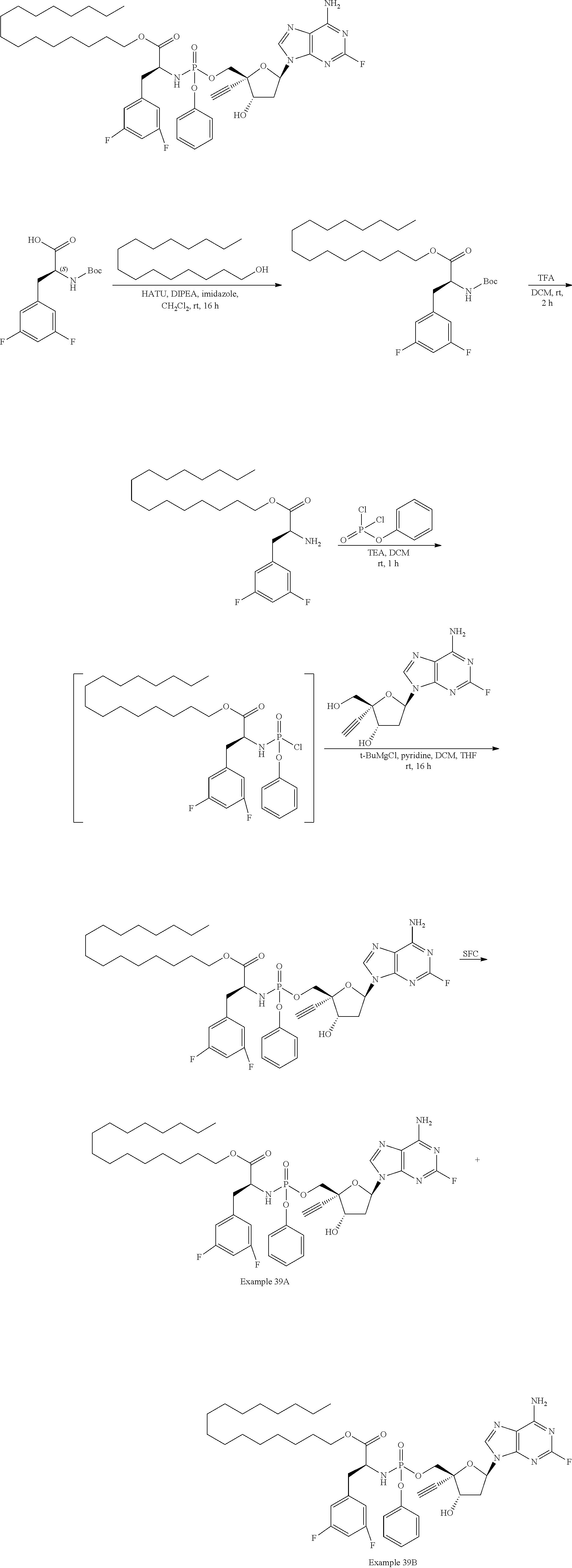

### Step 1: Hexadecyl (S)-2-((tert-butoxycarbonyl)amino)-3-(3,5-difluorophenyl)propanoate

To a solution of hexadecan-1-ol (8.85 g, 36.5 mmol), (S)-2-((tert-butoxycarbonyl)amino)-3-(3,5-difluorophenyl)propanoic acid (10 g, 33.2 mmol) and HATU (18.93 g, 49.8 mmol) in DCM (100 mL) was added imidazole (6.78 g, 100 mmol) and DIPEA (17.39 mL, 100 mmol). The resulting mixture was stirred at 25° C. overnight. TLC (pet. ether:EtOAc=10:1, Rf=0.5) indicated completion of reaction. The reaction was quenched with water (100 mL) and DCM (50 mL), and the organic layer was separated. The aqueous layer was extracted with DCM (2×50 mL). The combined organic layers were washed with brine (200 mL), dried with Na₂SO₄ and concentrated to give crude product. The residue was purified by combiflash (silica gel column 330 g, hexane:ethyl acetate=20:1). The appropriate fractions containing product were combined and the solvent was removed in vacuum to give hexadecyl (S)-2-((tert-butoxycarbonyl)amino)-3-(3,5-difluorophenyl)propanoate (8.5 g, 16.17 mmol, 48.7% yield) as yellow solid. ¹H NMR (400 MHz, MeOD) δ 6.87 (d, J=6.6 Hz, 2H), 6.82 (dd, J=14.9, 5.6 Hz, 1H), 4.37 (dd, J=9.2, 5.8 Hz, 1H), 4.13 (t, J=6.5 Hz, 2H), 3.15 (dd, J=13.8, 5.7 Hz, 1H), 2.94 (dd, J=13.7, 9.4 Hz, 1H), 1.67-1.57 (m, 2H), 1.41 (s, 9H), 1.37 (d, J=12.3 Hz, 2H), 1.31 (s, 24H), 0.92 (t, J=6.8 Hz, 3H).

### Step 2: Hexadecyl (S)-2-amino-3-(3,5-difluorophenyl)propanoate

To a solution of hexadecyl (S)-2-((tert-butoxycarbonyl)amino)-3-(3,5-difluorophenyl)propanoate (8.5 g, 16.17 mmol) in DCM (100 mL) was added TFA (7.47 mL, 97 mmol). The resulting mixture was stirred at 25° C. 2 h. TLC (pet. ether:EtOAc=10:1, Rf=0.05) indicated completion of reaction. The reaction was diluted with water (200 mL) and DCM (100 mL). The organic layer was separated and the aqueous layer was extracted with DCM (2×100 mL). The combined organic layers were washed with brine (50 mL), dried with Na₂SO₄ and concentrated to give crude hexadecyl (S)-2-amino-3-(3,5-difluorophenyl)propanoate (6.3 g, 14.80 mmol, 92% yield) which was used in the next step without purification.

### Step 3: Hexadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a cold (ice-water bath) solution of hexadecyl (S)-2-amino-3-(3,5-difluorophenyl)propanoate (3.99 g, 9.38 mmol), triethylamine (1.307 mL, 9.38 mmol) in DCM (60 mL) was added phenyl phosphorodichloridate (1.978 g, 9.38 mmol) in DCM (10 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1.1 g, 3.75 mmol) in THF (30 mL)/pyridine (15 mL) was added tert-butylmagnesium chloride (9.38 mL, 9.38 mmol) dropwise under an atmosphere of nitrogen, and the mixture was stirred at room temperature for 30 min. The first prepared mixture was added dropwise to the second prepared mixture at room temperature and stirred for 2 h. LCMS indicated completion of reaction. The mixture was filtered and concentrated to afford yellow solid. Then, the solid was dissolved in EtOAc (40 mL) and partitioned with HCl (0.05 N, 20 mL). The layers were separated, and the aqueous layer was extracted with EtOAc (40 mL). The combined organic phases were washed with brine, dried over Na₂SO₄, filtered and concentrated to afford the crude product. The residue was purified by combiflash (silica gel column 20 g, DCM:MeOH=24:1). The appropriate fractions containing product were combined and the solvent was removed in vacuum to give hexadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (500 mg, 0.583 mmol, 15.56% yield) as yellow solid. LCMS (M+H)=857.1; Retention time (0.1% TFA)=2.52 min; The diastereomers were separated by SFC (Instrument: SFC-80 (Thar, Waters), Column: OZ 20×250 mm, 10 μm (Deicel), Column temperature: 40° C.; Mobile phase: CO₂/ETOH(1.0% ammonia/methanol)=60/40; Flow rate: 80 g/min; Back pressure: 100 bar; Detection wavelength: 259 nm; Cycle time: 9 min) to afford first eluting isomer (Example 39A, RT1: 1.67 min, 200 mg, 0.233 mmol, 40.0% yield) as white solid; LCMS (M+H)=857.3; Retention time (0.1% TFA)=2.512 min; HPLC: Retention time (0.1% TFA)=13.439 min; ¹H NMR (400 MHz, DMSO) δ 8.18 (s, 1H), 7.84 (s, 2H), 7.22 (t, J=7.9 Hz, 2H), 7.09 (t, J=7.4 Hz, 1H), 6.97 (s, 1H), 6.95 (s, 1H), 6.88 (d, J=6.3 Hz, 2H), 6.26 (dd, J=7.4, 5.1 Hz, 1H), 6.15 (dd, J=12.3, 10.5 Hz, 1H), 5.78 (d, J=5.5 Hz, 1H), 4.55 (dd, J=12.6, 6.9 Hz, 1H), 4.02 (dd, J=9.8, 5.2 Hz, 2H), 3.93 (ddd, J=13.8, 10.8, 4.2 Hz, 3H), 3.64 (s, 1H), 2.95 (d, J=11.0 Hz, 1H), 2.80 (dd, J=13.7, 9.0 Hz, 1H), 2.69 (dd, J=12.0, 6.3 Hz, 1H), 2.43 (dd, J=13.6, 7.2 Hz, 2H), 1.43 (s, 2H), 1.19 (d, J=23.9 Hz, 26H), 0.85 (t, J=6.8 Hz, 3H) and second eluting isomer (Example 39B, RT2: 2.82 min, 200 mg, 0.233 mmol, 40.0% yield) was obtained as white solid. LCMS (M+H)=857.3; Retention time (0.1% TFA)=2.512 min; HPLC: Retention time (0.1% NH₄HCO₃)=13.736 min. LCMS (M+H)=857.3; Retention time (0.1% TFA)=2.52 min; HPLC: Retention time (0.1% TFA)=13.39 min; ¹H NMR (400 MHz, DMSO) δ 8.22 (s, 1H), 7.83 (s, 2H), 7.25 (t, J=7.9 Hz, 2H), 7.11 (t, J=7.2 Hz, 1H), 7.01 (d, J=8.5 Hz, 2H), 6.91 (d, J=8.6 Hz, 2H), 6.23 (dd, J=7.6, 4.8 Hz, 1H), 6.14 (dd, J=12.7, 10.6 Hz, 1H), 5.74 (d, J=5.6 Hz, 1H), 4.52 (dd, J=12.9, 7.2 Hz, 1H), 4.06 (dd, J=10.8, 6.4 Hz, 1H), 3.98 (dd, J=9.4, 6.0 Hz, 1H), 3.89-3.83 (m, 1H), 3.82-3.75 (m, 1H), 3.60 (s, 1H), 3.36 (dd, J=11.7, 6.5 Hz, 1H), 2.96 (dd, J=12.1, 4.6 Hz, 1H), 2.80 (dd, J=13.5, 9.1 Hz, 1H), 2.73-2.65 (m, 1H), 2.43 (dd, J=13.5, 7.5 Hz, 2H), 1.40-1.35 (m, 2H), 1.23 (d, J=4.9 Hz, 26H), 0.86-0.83 (m, 3H).

### Example 40

### Octadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)poropanoate

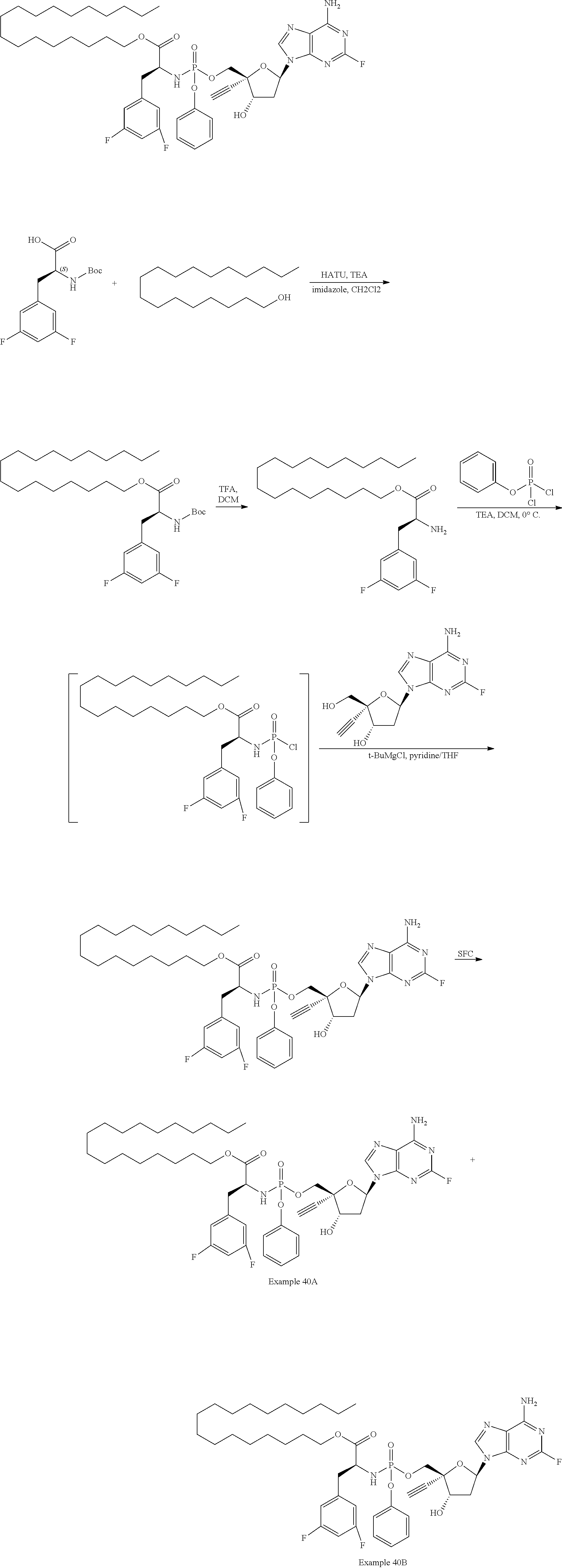

### Step-1: Octadecyl (S)-2-((tert-butoxycarbonyl)amino)-3-(3,5-difluorophenyl)propanoate

A mixture of octadecan-1-ol (8.98 g, 33.2 mmol), (S)-2-((tert-butoxycarbonyl)amino)-3-(3,5-difluorophenyl)propanoic acid (10 g, 33.2 mmol), imidazole (6.78 g, 100 mmol), DIPEA (17.39 mL, 100 mmol) and HATU (18.93 g, 49.8 mmol) in DCM (500 mL was stirred at 25° C. for 16 hs. TLC showed presence of new product. Water (500 mL) was added and the resulting mixture was extracted with DCM (250 mL×3). The combined organic phases were washed with brine, dried over Na₂SO₄, filtered and concentrated. The residue was purified by silica gel chromatography (pet. ether:EtOAc=30: 1) to afford octadecyl (S)-2-((tert-butoxycarbonyl)amino)-3-(3,5-difluorophenyl)propanoate (16 g, 28.9 mmol, 87% yield) as white solid. ¹H NMR (400 MHz, DMSO) δ 7.34-6.98 (m, 3H), 4.21-4.01 (m, 3H), 3.03-2.84 (m, 3H), 2.83 (qd, J=13.5, 7.0 Hz, 2H), 1.49 (s, 3H), 1.39 (s, 9H), 1.23 (s, 30H), 0.84 (t, J=6.8 Hz, 3H).

### Step-2: Octadecyl (S)-2-amino-3-(3,5-difluorophenyl)propanoate

To a solution of (octadecyl (S)-2-((tert-butoxycarbonyl)amino)-3-(3,5-difluorophenyl)propanoate (16 g, 28.9 mmol) in DCM (100 mL) was added TFA (95 mL, 1228 mmol) at 25° C. The reaction mixture was stirred at 25° C. for 4 h. TLC showed starting material was consumed completely. The reaction was concentrated to dryness and the pH of the residue was adjusted to 5-6 with Na₂CO₃ solution, and then extracted with DCM (200 mL×3). The combined organic layers were washed with brine, dried over Na₂SO₄, filtered and concentrated. The residue was purified by silica gel chromatography to afford octadecyl (S)-2-amino-3-(3,5-difluorophenyl)propanoate (8.0 g, 17.63 mmol, 61.0% yield). ¹H NMR (400 MHz, DMSO) δ 7.14-6.83 (m, 3H), 4.08-3.85 (m, 2H), 3.60 (t, J=6.9 Hz, 1H), 2.83 (qd, J=13.5, 7.0 Hz, 2H), 1.48 (d, J=6.6 Hz, 2H), 1.23 (s, 30H), 0.85 (t, J=6.8 Hz, 3H).

### Step-3: Octadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a solution of octadecyl (S)-2-amino-3-(3,5-difluorophenyl)propanoate (1.547 g, 3.41 mmol), triethylamine (1.188 mL, 8.52 mmol) in DCM (48 mL) was added phenyl phosphorodichloridate (1.799 g, 8.52 mmol) in DCM (3 mL) dropwise under an atmosphere of nitrogen at 0° C., and then the reaction mixture was stirred at room temperature for 1 h. Meanwhile to a solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (24 mL)/pyridine (12 mL) was added tert-butylmagnesium chloride (8.52 mL, 8.52 mmol) dropwise under an atmosphere of nitrogen at 0° C., and then the mixture was warmed and stirred at room temperature for 30 min. The first prepared mixture was added dropwise to the second prepared mixture at room temperature. The mixture was stirred at room temperature for 2 h. LCMS indicated the completion of reaction. The reaction mixture was filtered, concentrated, and then the residue was diluted with DCM. The mixture was added water and organic layer separated, washed with 0.5 N HCl three times and brine. The organic phase was dried over Na₂SO₄, filtered and concentrated. The residue was purified by flash chromatography (0-5% MeOH/DCM) to give octadecyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (450 mg, 0.483 mmol, 14.17% yield). LCMS (M+H)=885.7, Retention time (0.1% TFA)=3.40 min. The diastereomers were separated with Prep-SFC (Instrument: SFC-80 (Thar, Waters), Column: OZ 20×250 mm, 10 μm (Daicel), Column temperature: 40° C.; Mobile phase: CO₂/MEOH(0.2% ammonia/methanol)=50/50; Flow rate: 80 g/min; Back pressure: 100 bar; Detection wavelength: 260 nm; Cycle time: 9 min) to give first eluting isomer (Example 40A, RT1: 1.92 min, 218.5 mg, 0.2469 mmol, 100% 24.71% yield) as an off-white solid; LCMS (M+H)=885.7; Retention time (0.1% TFA)=3.40 min. HPLC: Retention time (0.05% TFA)=12.337 min. ¹H NMR (400 MHz, MeOD) δ 8.14 (s, 1H), 7.27 (t, J=7.9 Hz, 2H), 7.21-7.03 (m, 3H), 6.76 (dd, J=8.4, 5.7 Hz, 3H), 6.36 (dd, J=7.5, 4.3 Hz, 1H), 4.75 (t, J=7.4 Hz, 1H), 4.20 (ddd, J=37.6, 11.0, 5.2 Hz, 2H), 4.09-3.94 (m, 3H), 3.22 (s, 1H), 3.03 (ddd, J=13.6, 5.8, 2.5 Hz, 1H), 2.90-2.74 (m, 2H), 2.64 (dd, J=14.4, 6.8 Hz, 1H), 1.63-1.40 (m, 2H), 1.37-1.15 (m, 32H), 0.91 (t, J=6.8 Hz, 3H) and second eluting isomer (Example 40B, RT2: 3.33 min, 100 mg, 0.1389 mmol, 99.67%, 20.71% yield) as an off-white solid; LCMS (M+H)=885.7; Retention time (0.1% TFA)=3.42 min. HPLC: Retention time (0.05% TFA)=15.548 min. ¹H NMR (400 MHz, MeOD) δ 8.11-7.87 (m, 1H), 7.12 (t, J=7.7 Hz, 2H), 7.02-6.89 (m, 3H), 6.76-6.51 (m, 3H), 6.22 (dd, J=7.1, 4.3 Hz, 1H), 4.68-4.48 (m, 1H), 4.11 (dd, J=10.8, 6.1 Hz, 1H), 4.01 (dd, J=12.7, 6.7 Hz, 2H), 3.84 (ddd, J=33.5, 20.7, 14.4 Hz, 2H), 3.06 (d, J=10.5 Hz, 1H), 2.92 (dd, J=13.5, 5.7 Hz, 1H), 2.76 (dd, J=13.6, 8.4 Hz, 1H), 2.71-2.61 (m, 1H), 2.58-2.49 (m, 1H), 1.50-1.25 (m, 2H), 1.14 (d, J=15.7 Hz, 32H), 0.78 (t, J=6.4 Hz, 3H).

### Example 41

### Icosyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

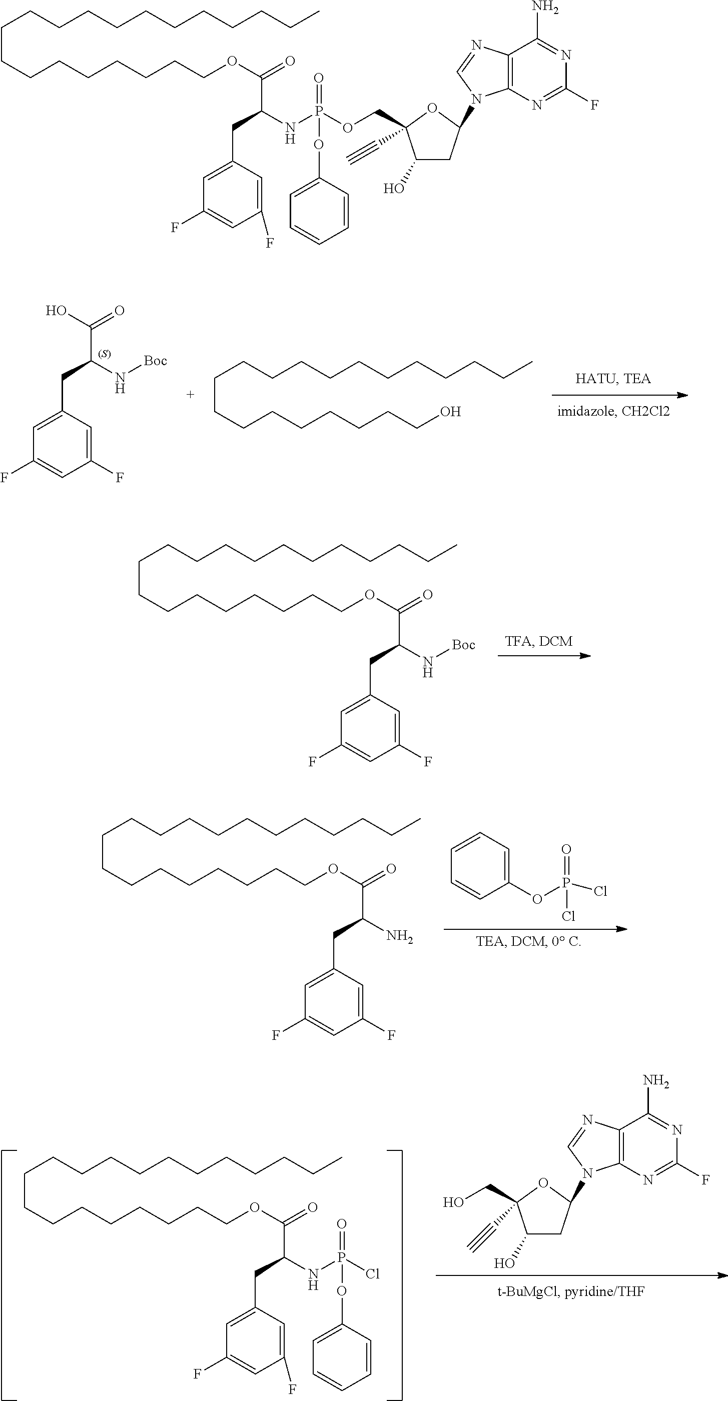

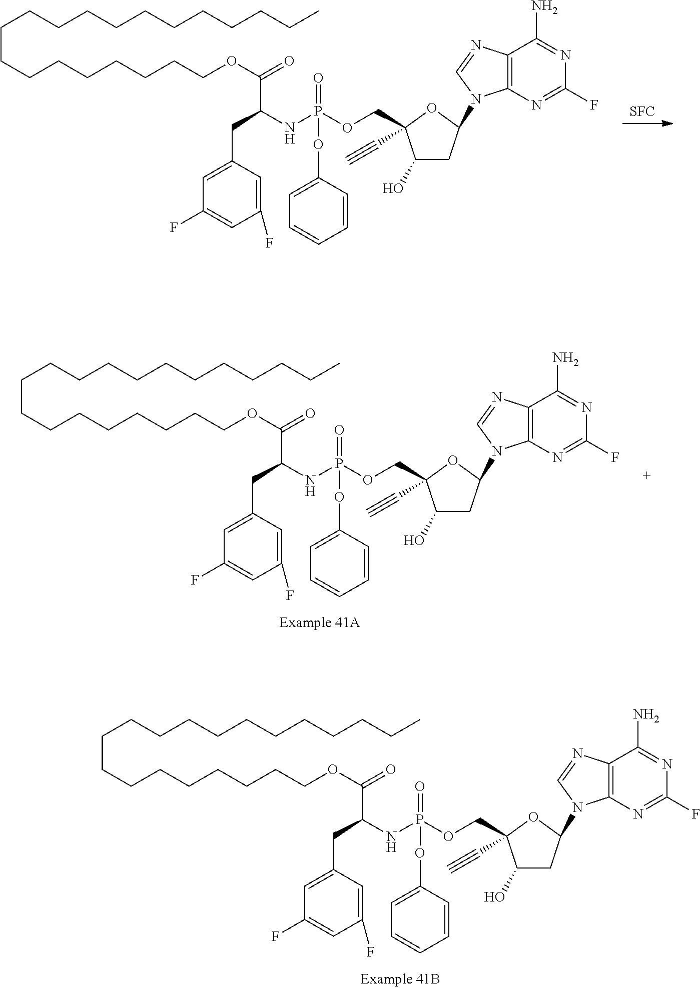

### Step-1: Icosyl (S)-2-((tert-butoxycarbonyl)amino)-3-(3,5-difluorophenyl)propanoate

A mixture of icosan-1-ol (9.91 g, 33.2 mmol), (S)-2-((tert-butoxycarbonyl)amino)-3-(3,5-difluorophenyl)propanoic acid (10 g, 33.2 mmol), imidazole (6.78 g, 100 mmol), DIPEA (17.39 mL, 100 mmol) and HATU (18.93 g, 49.8 mmol) in DCM (500 mL) was stirred at 25° C. for 16 h. TLC showed the presence of new product. Water (500 mL) was added and the reaction mixture was extracted with DCM (250 mL×3). The combined organic phases were washed with brine, dried over Na₂SO₄, filtered and concentrated. The residue was purified by silica gel chromatography (pet. ether:EtOAc=30:1) to afford icosyl (S)-2-((tert-butoxycarbonyl)amino)-3-(3,5-difluorophenyl)propanoate (14 g, 24.06 mmol, 72.5% yield) as white solid. ¹H NMR (400 MHz, DMSO) δ 7.36-7.34 (m, 1H), 7.06-6.98 (m, 3H), 4.02-3.99 (m, 3H), 2.88 (m, 3H), 1.51-1.48 (m, 2H), 1.32 (s, 9H), 1.27 (S, 34H), 0.86-0.83 (m, 3H).

### Step-2: Icosyl (S)-2-amino-3-(3,5-difluorophenyl)propanoate

To a solution of (icosyl (S)-2-((tert-butoxycarbonyl)amino)-3-(3,5-difluorophenyl)propanoate (14 g, 24.06 mmol) in DCM (50 mL) was added TFA (50 mL, 649 mmol) at 25° C. The reaction mixture was stirred at 25° C. for 4 h. TLC showed the reaction was completed. The reaction was concentrated to dryness. The pH of the residue was adjusted to 5-6 with Na₂CO₃ and extracted with DCM (100 mL×3). The combined organic layers were washed with brine, dried over Na₂SO₄, filtered and concentrated. The residue was purified by silica gel chromatography to afford icosyl (S)-2-amino-3-(3,5-difluorophenyl)propanoate (8 g, 16.61 mmol, 69.0% yield). ¹H NMR (400 MHz, DMSO) δ 7.11-6.84 (m, 3H), 3.98 (dd, J=11.6, 5.9 Hz, 2H), 3.60 (t, J=6.9 Hz, 1H), 2.94-2.72 (m, 2H), 2.07 (s, 2H), 1.47 (s, 2H), 1.23 (s, 34H), 0.84 (d, J=6.8 Hz, 3H).

### Step-3: Icosyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a solution of icosyl (S)-2-amino-3-(3,5-difluorophenyl)propanoate (1.643 g, 3.41 mmol), triethylamine (1.188 mL, 8.52 mmol) in DCM (48 mL) was added phenyl phosphorodichloridate (1.799 g, 8.52 mmol) in DCM (3 mL) dropwise under an atmosphere of nitrogen at 0° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (1 g, 3.41 mmol) in THF (20 mL)/pyridine (10 mL) was added tert-butylmagnesium chloride (8.52 mL, 8.52 mmol) dropwise under an atmosphere of nitrogen at 0° C., and then the mixture was warmed and stirred at room temperature for 30 min. The first prepared mixture was added dropwise to the second prepared mixture at room temperature and stirred for 2 h. LCMS indicated the completion of reaction. The reaction mixture was filtered, concentrated, and the residue was diluted with DCM. The mixture was added water and then extracted. The organic layer was washed with 0.5 N HCl three times and brine. The organic phase was dried over Na₂SO₄, filtered and concentrated. The residue was purified by flash chromatography (DCM:MeOH=0-5%) to give icosyl (2S)-2-(((((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate (400 mg, 0.438 mmol, 12.85% yield). LCMS (M+H)=914.7; Retention time (0.1% TFA)=3.82 min. The diastereomers were separated with Prep-SFC (Instrument: SFC-80 (Thar, Waters), Column: OZ 20×250 mm, 10 μm (Daicel), Column temperature: 40° C.; Mobile phase: CO₂/ETOH(1.0% ammonia/methanol)=55/45; Flow rate: 80 g/min; Back pressure: 100 bar; Detection wavelength: 260 nm; Cycle time: 9 min) to give first eluting isomer (example 41A, RT1: 1.3 min, 112.6 mg, 0.1231 mmol, 100% 20.71% yield) as an off-white solid; LCMS (M+H)=914.7; Retention time (0.1% TFA)=3.82 min. HPLC: Retention time (0.1% NH₄HCO₃)=12.222 min. ¹H NMR (400 MHz, MeOD) δ 8.14 (s, 1H), 7.27 (t, J=7.9 Hz, 2H), 7.19-7.04 (m, 3H), 6.76 (dd, J=8.4, 5.7 Hz, 3H), 6.36 (dd, J=7.5, 4.3 Hz, 1H), 4.74 (d, J=7.4 Hz, 1H), 4.25 (dd, J=11.0, 5.2 Hz, 1H), 4.15 (dd, J=11.0, 5.2 Hz, 1H), 4.08-3.97 (m, 3H), 3.22 (s, 1H), 3.03 (ddd, J=13.6, 5.8, 2.5 Hz, 1H), 2.91-2.72 (m, 2H), 2.71-2.59 (m, 1H), 1.53 (d, J=5.6 Hz, 2H), 1.36-1.19 (m, 36H), 0.91 (t, J=6.8 Hz, 3H) and second eluting isomer (Example 41B, RT2: 2.13 min, 109.5 mg, 0.1197 mmol, 99.71%, 19.74% yield) as an off-white solid; LCMS (M+H)=914.7; Retention time (0.1% TFA)=3.84 min. HPLC: Retention time (0.1% NH₄HCO₃)=13.861 min. ¹H NMR (400 MHz, MeOD) δ 8.14 (d, J=6.0 Hz, 1H), 7.24 (t, J=7.9 Hz, 2H), 7.14-7.01 (m, 3H), 6.88-6.66 (m, 3H), 6.32 (dd, J=7.6, 4.2 Hz, 1H), 4.72 (dd, J=18.3, 10.7 Hz, 1H), 4.20 (dd, J=11.0, 6.2 Hz, 1H), 4.14-4.01 (m, 2H), 3.98 (qt, J=10.7, 6.6 Hz, 2H), 3.17 (d, J=8.6 Hz, 1H), 3.02 (ddd, J=13.6, 6.1, 2.2 Hz, 1H), 2.92-2.71 (m, 2H), 2.63 (dt, J=13.6, 7.7 Hz, 1H), 1.51 (d, J=6.2 Hz, 2H), 1.39-1.19 (m, 36H), 0.90 (t, J=6.8 Hz, 3H).

### Example 42

### Docosyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

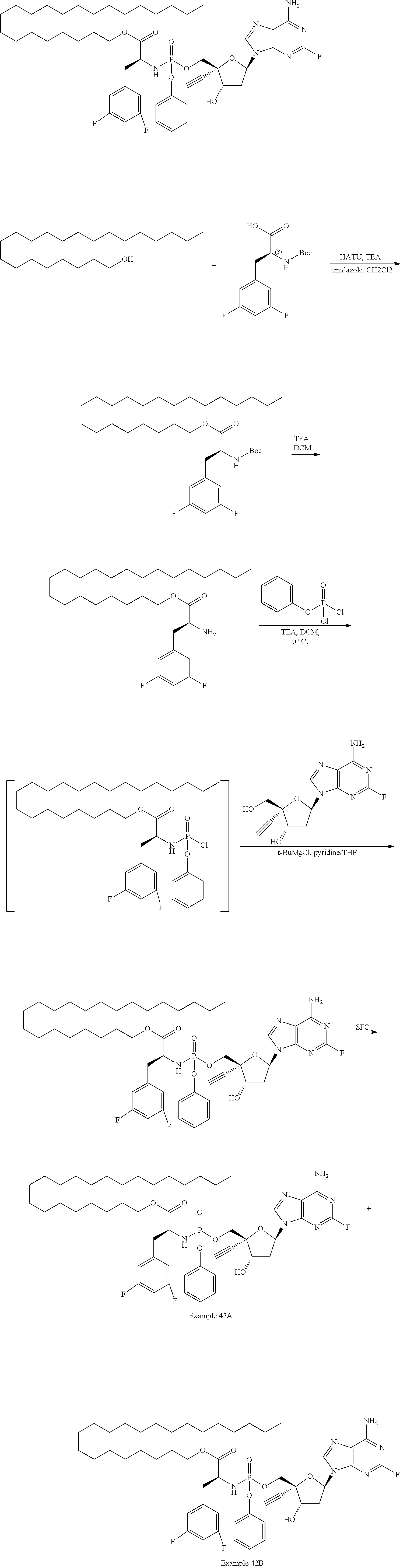

### Step-1: Docosyl (S)-2-((tert-butoxycarbonyl)amino)-3-(3,5-difluorophenyl)propanoate

A mixture of docosan-1-ol (10.84 g, 33.2 mmol), (S)-2-((tert-butoxycarbonyl)amino)-3-(3,5-difluorophenyl)propanoic acid (10 g, 33.2 mmol), imidazole (6.78 g, 100 mmol), DIPEA (17.39 mL, 100 mmol) and HATU (18.93 g, 49.8 mmol) in DCM (500 mL) was stirred at 25° C. for 16 h. TLC showed the presence of new product. Water (500 mL) was added and the reaction mixture was extracted with DCM (250 mL×3). The combined organic phases were washed with brine, dried over Na₂SO₄, filtered and concentrated. The residue was purified by silica gel chromatography (pet. ether:EtOAc=30:1) to afford docosyl (S)-2-((tert-butoxycarbonyl)amino)-3-(3,5-difluorophenyl)propanoate (16 g, 26.2 mmol, 79 yield) as white solid. ¹H NMR (400 MHz, DMSO) δ 7.36-7.34 (m, 1H), 7.06-6.98 (m, 3H), 4.03-4.00 (m, 3H), 3.00-2.68 (m, 3H), 1.51-1.48 (m, 2H), 1.32 (s, 9H), 1.27 (S, 38H), 0.86-0.83 (m, 3H).

### Step-2: Docosyl (S)-2-amino-3-(3,5-difluorophenyl)propanoate

To a solution of docosyl (S)-2-((tert-butoxycarbonyl)amino)-3-(3,5-difluorophenyl)propanoate (10 g, 16.40 mmol) in DCM (50 mL) was added TFA (1.263 mL, 16.40 mmol) at 0° C. Then, the mixture was warmed to room temperature and stirred for 2 h. TLC showed completion of reaction. The pH of the reaction mixture was adjusted to 7-8 with sat. Na₂CO₃ solution and extracted with DCM (100 ml×3). The combined organic phases were washed with brine, dried over Na₂SO₄, filtered and concentrated to give the residue. The residue was purified by silica gel chromatography (DCM:MeOH=20:1) to give docosyl (S)-2-amino-3-(3,5-difluorophenyl)propanoate (8 g, 14.91 mmol, 91% yield). ¹H NMR (400 MHz, DMSO) δ 7.00 (d, J=34.2 Hz, 3H), 5.76 (s, 1H), 3.99 (s, 2H), 3.62 (s, 1H), 2.83 (s, 2H), 1.47 (s, 2H), 1.23 (s, 38H), 0.85 (s, 3H).

### Step-3: Docosyl (2S)-2-(((((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate

To a solution of docosyl (S)-2-amino-3-(3,5-difluorophenyl)propanoate (6.52 g, 12.79 mmol), triethylamine (1.782 mL, 12.79 mmol) in DCM (40 mL) was added phenyl phosphorodichloridate (2.70 g, 12.79 mmol) in DCM (1 mL) dropwise under an atmosphere of nitrogen at 0° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (1.5 g, 5.11 mmol) in THF (3 mL)/pyridine (1.5 mL) was added tert-butylmagnesium chloride (12.79 mL, 12.79 mmol) dropwise under an atmosphere of nitrogen at 0° C., and then the mixture was warmed to room temperature and stirred for 30 min. The first prepared mixture was added dropwise to the second prepared mixture at room temperature and stirred for 2 h. LCMS indicated the completion of reaction. The reaction mixture was filtered, concentrated, and then the residue was diluted with DCM. The mixture was added water and extracted. Then, the organic layer was washed with 0.5 N HCl three times and brine. The organic phase was dried over Na₂SO₄, filtered and concentrated. The residue was purified by flash chromatography (DCM:MeOH=0-5%) to give docosyl (2S)-2-(((((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (800 mg, 0.816 mmol, 15.95% yield). LCMS (M+H)=941.7; Retention time (0.1% TFA)=4.42 min. The diastereomers were separated with Prep-SFC (Instrument: SFC-80 (Thar, Waters), Column: IG 20×250 mm, 10 μm (Daicel), Column temperature: 40° C.; Mobile phase: CO₂/ETOH (1.0% ammonia/methanol)=40/60; Flow rate: 80 g/min; Back pressure: 100 bar; Detection wavelength: 260 nm; Cycle time: 10.5 min) to give first eluting isomer (Example 42A, RT1: 1.58 min, 231.6 mg, 0.2459 mmol, 100% 23.21% yield) as an off-white solid; LCMS (M+H)=941.7; Retention time (0.1% TFA)=4.42 min. HPLC: Retention time (0.05% TFA)=23.397 min. ¹H NMR (400 MHz, MeOD) δ 8.12 (s, 1H), 7.25 (t, J=7.9 Hz, 2H), 7.12 (t, J=7.0 Hz, 1H), 7.08-7.01 (m, 2H), 6.81-6.65 (m, 3H), 6.34 (dd, J=7.5, 4.3 Hz, 1H), 4.73 (t, J=7.4 Hz, 1H), 4.23 (dd, J=11.0, 5.2 Hz, 1H), 4.14 (dd, J=11.0, 5.2 Hz, 1H), 4.06-3.98 (m, 3H), 3.20 (s, 1H), 3.01 (ddd, J=13.7, 5.8, 2.6 Hz, 1H), 2.90-2.68 (m, 2H), 2.62 (dt, J=13.6, 7.6 Hz, 1H), 1.51 (d, J=6.0 Hz, 2H), 1.26 (d, J=18.0 Hz, 40H), 0.90 (t, J=6.9 Hz, 3H) and second eluting isomer (Example 42B, RT2: 2.77 min, 279.5 mg, 0.2968 mmol, 99.49%, 24.82% yield) as an off-white solid; LCMS (M+H)=941.7; Retention time (0.1% TFA)=4.43 min. HPLC: Retention time (0.05% TFA)=23.502 min. ¹H NMR (400 MHz, MeOD) δ 8.05 (d, J=18.2 Hz, 1H), 7.14 (t, J=7.9 Hz, 2H), 7.05-6.90 (m, 3H), 6.77-6.55 (m, 3H), 6.22 (dd, J=7.6, 4.2 Hz, 1H), 4.63 (t, J=7.5 Hz, 1H), 4.10 (dd, J=11.0, 6.2 Hz, 1H), 4.05-3.96 (m, 2H), 3.87 (tdd, J=9.8, 6.1, 3.7 Hz, 2H), 3.09 (d, J=6.6 Hz, 1H), 2.98-2.87 (m, 1H), 2.83-2.61 (m, 2H), 2.53 (dt, J=13.5, 7.8 Hz, 1H), 1.44-1.32 (m, 2H), 1.16 (d, J=15.7 Hz, 40H), 0.79 (t, J=6.8 Hz, 3H).

### Example 43

### Icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

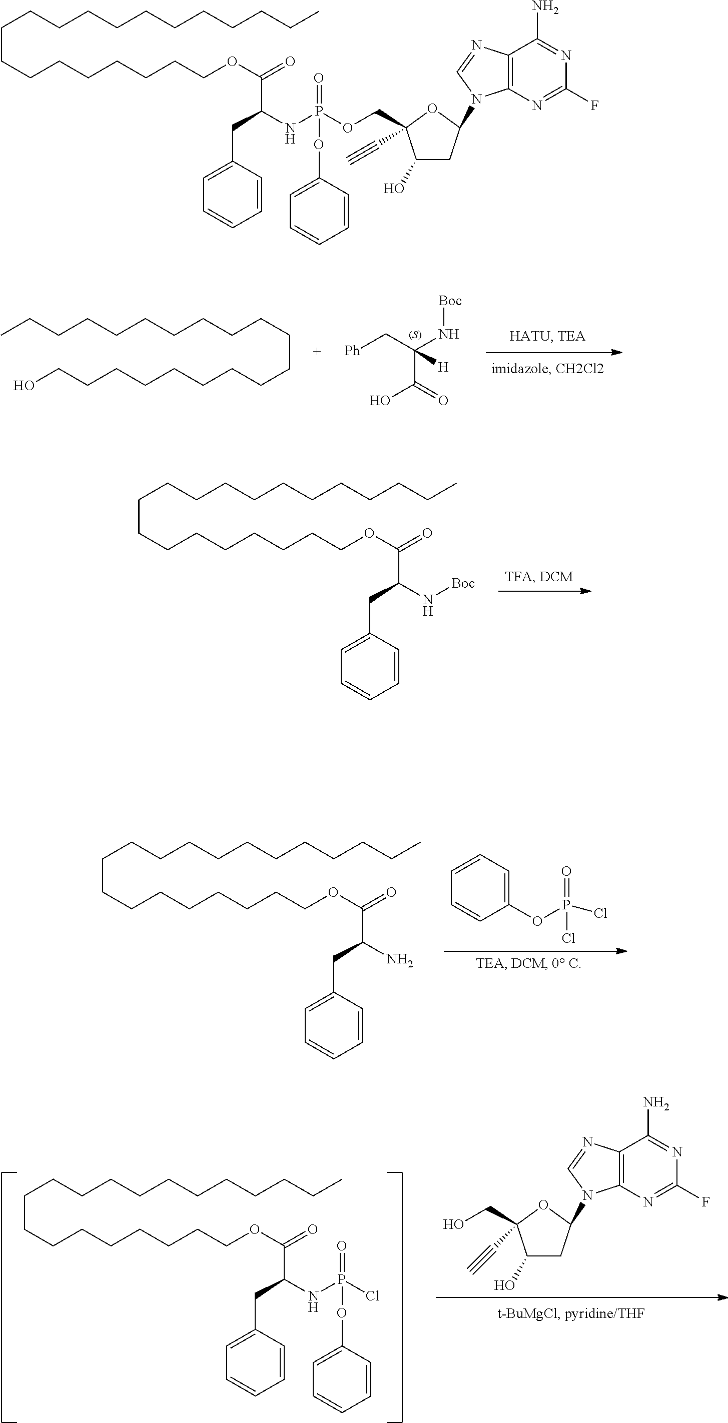

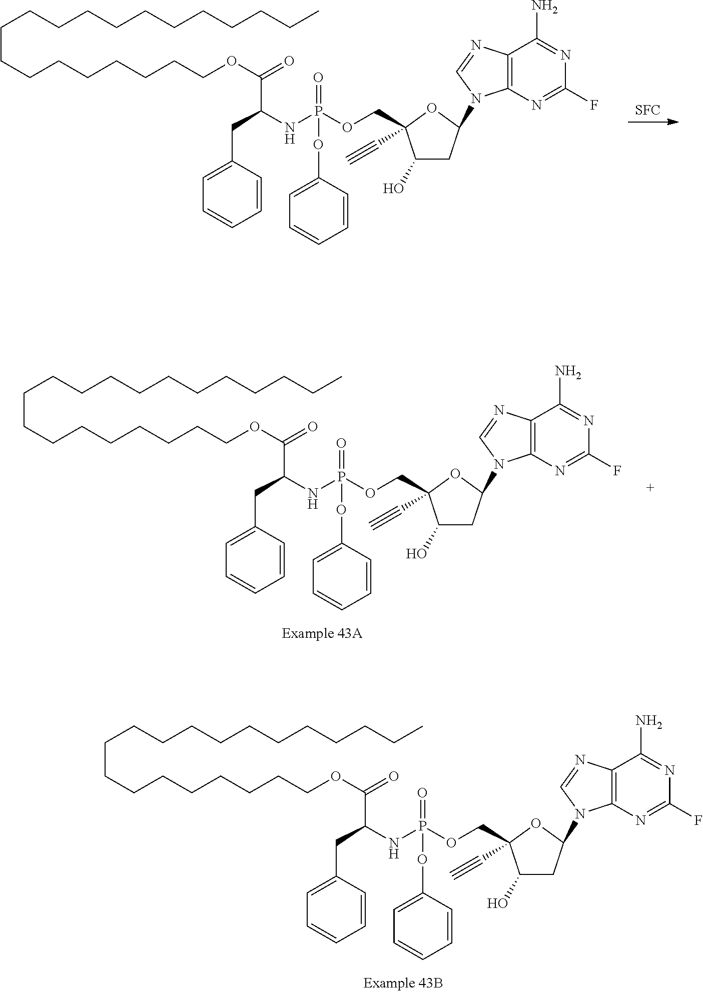

### Step-1: Icosyl (tert-butoxycarbonyl)-L-phenylalaninate

A mixture of icosan-1-ol (33.8 g, 113 mmol), (tert-butoxycarbonyl)-L-phenylalanine (25 g, 94 mmol), imidazole (19.25 g, 283 mmol), DIPEA (49.4 mL, 283 mmol) and HATU (53.7 g, 141 mmol) in DCM (200 mL) was stirred at 25° C. for 16 h. TLC showed the presence of new product. Water (200 mL) was added and the mixture was extracted with DCM (150 mL×3). The combined organic phases were washed with brine, dried over Na₂SO₄, filtered and concentrated. The residue was purified by silica gel chromatography (pet. ether:EtOAc=30:1) to afford icosyl (tert-butoxycarbonyl)-L-phenylalaninate (20 g, 10.99 mmol, 11.67% yield) as white solid. ¹H NMR (400 MHz, CDCl₃) δ 7.3-7.28 (m, 1H), 7.26-7.11 (m, 4H), 4.99 (d, J=7.4 Hz, 1H), 4.57 (d, J=7.3 Hz, 1H), 4.12-4.02 (m, 2H), 3.49 (s, 1H), 3.17-2.99 (m, 2H), 1.42 (s, 8H), 1.26 (s, 36H), 0.88 (t, J=6.8 Hz, 3H).

### Step-2: Icosyl L-phenylalaninate

To a solution of icosyl (tert-butoxycarbonyl)-L-phenylalaninate (30 g, 55.0 mmol) in DCM (200 mL) was added TFA (42.3 mL, 550 mmol) at 0° C. After 5 min, the mixture was warmed to room temperature and stirred for 2 h. TLC showed starting material was consumed completely. The pH of the reaction mixture was adjusted to 7-8 with sat. Na₂CO₃ solution and extracted with DCM (300 mL×3). The combined organic phases were washed with brine, dried over Na₂SO₄, filtered and concentrated to give the residue. The residue was purified by silica gel chromatography (DCM:MeOH=20:1) to give icosyl L-phenylalaninate (25 g, 50.5 mmol, 92% yield). ¹H NMR (400 MHz, DMSO) δ 7.34-7.08 (m, 5H), 4.00-3.88 (m, 2H), 3.55 (t, J=6.8 Hz, 1H), 2.92-2.73 (m, 2H), 1.91 (s, 2H), 1.52-1.39 (m, 2H), 1.23 (s, 35H), 0.85 (t, J=6.8 Hz, 3H).

### Step-3: Icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of icosyl L-phenylalaninate (7.60 g, 17.05 mmol), triethylamine (2.376 mL, 17.05 mmol) in DCM (48 mL) was added phenyl phosphorodichloridate (3.60 g, 17.05 mmol) in DCM (1 mL) dropwise under an atmosphere of nitrogen in ice water, then the reaction mixture was stirred at room temperature for 1 h. To a solution of (2R,3S,5R)-5-(6-amino-2-fluoro-9H-purin-9-yl)-2-ethynyl-2-(hydromethyl)tetrahydrofuran-3-ol (2 g, 6.82 mmol) in THF (24 mL)/pyridine (12 mL) was added tert-butylmagnesium chloride (17.05 mL, 17.05 mmol) dropwise under an atmosphere of nitrogen at 0° C., and then the mixture was warmed to room temperature and stirred for 30 min. The first prepared mixture was added dropwise to the second prepared mixture at room temperature and stirred for 2 h. LCMS indicated the completion of reaction. The reaction mixture was filtered, concentrated, and then the residue was diluted with DCM. The mixture was added water and extracted. Then, the organic layer was washed with 0.5 N HCl three times and brine. The organic phase was dried over Na₂SO₄, filtered and concentrated. The residue was purified by flash chromatography (0-5% MeOH/DCM) to give icosyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (800 mg, 0.867 mmol, 12.71% yield). LCMS (M+H)=877.7, Retention time (0.1% TFA)=3.82 min. The diastereomers were separated with Prep-SFC (Instrument: SFC-80 (Thar, Waters), Column: OZ 20×250 mm, 10 μm (Deicel), Column temperature: 60° C.; Mobile phase: CO₂/ETOH (1.0% ammonia/methanol)=60/40; Flow rate: 80 g/min; Back pressure: 100 bar; Detection wavelength: 260 nm; Cycle time: 9 min) to give first eluting isomer (Example 43A, RT1: 1.35 min, 125.8 mg, 0.1434 mmol, 100.0%, yield: 16.79%) as an off-white solid; LCMS (M+H)=877.7, Retention time (0.1% TFA)=3.82 min. HPLC: Retention time (0.1% NH₄HCO₃)=13.350 min. ¹H NMR (400 MHz, MeOD) δ 8.14 (d, J=18.6 Hz, 1H), 7.33-7.18 (m, 5H), 7.16-6.99 (m, 5H), 6.42-6.28 (m, 1H), 4.76-4.65 (m, 1H), 4.17 (dt, J=37.1, 18.6 Hz, 1H), 4.02 (ddd, J=23.5, 12.5, 5.9 Hz, 4H), 2.98 (dt, J=15.3, 7.5 Hz, 1H), 2.86 (dd, J=11.8, 6.5 Hz, 1H), 2.80-2.59 (m, 2H), 1.47 (dd, J=18.5, 12.9 Hz, 2H), 1.39-1.08 (m, 36H), 0.91 (t, J=6.4 Hz, 3H) and second eluting isomer (Example 43B, RT2: 2.04 min, 207.5 mg, 0.2366 mmol, 99.56%, yield: 17.10%) as an off-white solid; LCMS (M+H)=877.7, Retention time (0.1% TFA)=3.84 min. HPLC: Retention time (0.1% NH₄HCO₃)=12.581 min. ¹H NMR (400 MHz, MeOD) δ 8.06-7.96 (m, 1H), 7.18-6.89 (m, 10H), 6.24-6.12 (m, 1H), 4.58 (t, J=7.5 Hz, 1H), 4.04-3.94 (m, 2H), 3.91-3.73 (m, 3H), 3.08-3.03 (m, 1H), 2.91 (ddd, J=13.5, 6.3, 2.2 Hz, 1H), 2.84-2.40 (m, 3H), 1.4-1.27 (m, 2H), 1.23-1.08 (m, 36H), 0.79 (t, J=6.9 Hz, 3H).

### Example 44

12,12,13,13,14,14,15,15,15-Nonafluoropentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

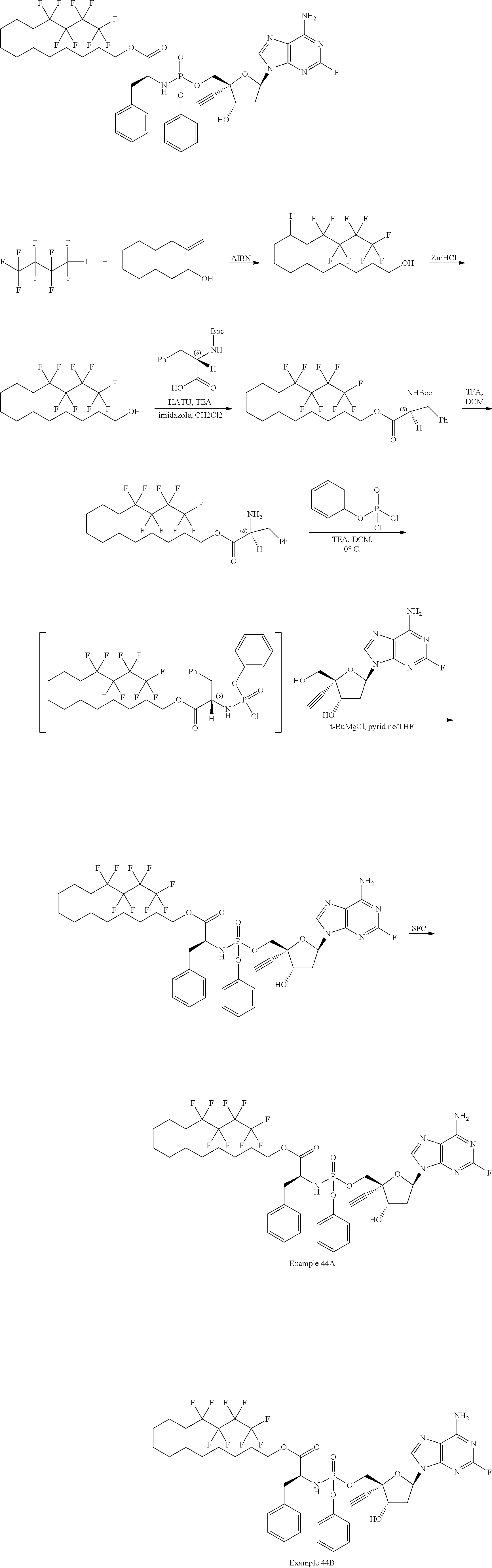

### Step-1: 12,12,13,13,14,14,15,15,15-Nonafluoro-10-iodopentadecan-1-ol

A mixture of 1,1,1,2,2,3,3,4,4-nonafluoro-4-iodobutane (40 g, 116 mmol), undec-10-en-1-ol (21.66 g, 127 mmol) and AIBN (1.899 g, 11.56 mmol) was heated at 65° C. in a sealed glass tube filled with nitrogen gas while gentle stirring. After 12 h, the resulting mixture was distilled under vacuum to give 12,12,13,13,14,14,15,15,15-nonafluoro-10-iodopentadecan-1-ol (50 g, 97 mmol, 84% yield). ¹H NMR (400 MHz, CDCl₃): δ 4.35 (m, 1H), 3.53 (t, J=16.0, 6.4 Hz, 2H), 2.97-2.86 (m, 2H), 1.88-1.73 (m, 2H), 1.49-1.42 (m, 2H), 1.33 (s, 14H).

### Step-2: 12,12,13,13,14,14,15,15,15-Nonafluoropentadecan-1-ol

To a slurry of zinc (28.5 g, 436 mmol) in ethanol (50 mL) was added AcOH (1.386 mL, 24.21 mmol). A solution of 12,12,13,13,14,14,15,15,15-nonafluoro-10-iodopentadecan-1-ol (50 g, 97 mmol) in ethanol (10.0 mL) was added dropwise with stirring over 1 hour, and the reaction mixture was heated at 50° C. for 4 h. TLC showed the presence of new compound. The reaction was concentrated to dryness. The pH of the residue was adjusted to 5-6 with aqueous Na₂CO₃, and extracted with EtOAc (50 mL×3). The combined organic layers were washed with brine, dried over Na₂SO₄, filtered and concentrated. The crude product was purified by silica gel chromatography to afford 12,12,13,13,14,14,15,15,15-nonafluoropentadecan-1-ol (21 g, 53.8 mmol, 55.5% yield). ¹H NMR (400 MHz, CDCl₃): δ 3.54 (t, J=17.0, 6.8 Hz, 2H), 2.16-2.12 (m, 2H), 1.62-1.51 (m, 4H), 1.33 (s, 14H).

### Step-3: 12,12,13,13,14,14,15,15,15-Nonafluoropentadecyl (tert-butoxycarbonyl)-L-phenylalaninate

A mixture of 12,12,13,13,14,14,15,15,15-nonafluoropentadecan-1-ol (22.07 g, 56.5 mmol), (tert-butoxycarbonyl)-L-phenylalanine (15 g, 56.5 mmol), imidazole (11.55 g, 170 mmol), DIPEA (29.6 mL, 170 mmol) and HATU (32.2 g, 85 mmol) in DCM (500 mL) was stirred at 25° C. for 16 h. TLC showed the presence of new compound. The reaction mixture was diluted with water (500 mL) and extracted with DCM (250 mL×3). The combined organic phases were washed with brine, dried over Na₂SO₄ and concentrated. The residue was purified by chromatography (pet. ether:EtOAc=1:1) to afford 12,12,13,13,14,14,15,15,15-nonafluoropentadecyl (tert-butoxycarbonyl)-L-phenylalaninate (18 g, 28.2 mmol, 49.9% yield) as white solid. ¹H NMR (400 MHz, CDCl₃): δ 7.29-7.27 (m, 3H), 7.13 (d, J=17.0, 6.8 Hz, 2H), 5.04 (m, 1H), 4.52 (m, 1H), 4.08 (t, J=15.0, 6.0 Hz, 2H), 3.09-3.06 (m, 2H), 2.06-2.01 (m, 2H), 1.61-1.57 (m, 2H), 1.41 (s, 14H), 1.28 (s, 9H).

### Step-4: 12,12,13,13,14,14,15,15,15-Nonafluoropentadecyl L-phenylalaninate

To a solution of 12,12,13,13,14,14,15,15,15-nonafluoropentadecyl (tert-butoxycarbonyl)-L-phenylalaninate (18 g, 28.2 mmol) in DCM (10 mL) was added TFA (10 mL, 130 mmol) at 25° C. and stirred for 4 h. TLC showed completion of reaction. The reaction was concentrated to dryness. The pH of the residue was adjusted to 5-6 with aqueous Na2CO3, and extracted with EtOAc (50 mL×3). The obtained organic layers were washed with brine, dried over Na₂SO₄, filtered and concentrated. The crude product was purified by silica gel chromatography to afford 12,12,13,13,14,14,15,15,15-nonafluoropentadecyl L-phenylalaninate (12 g, 22.33 mmol, 79% yield). ¹H NMR (400 MHz, CDCl₃): δ 7.31-7.26 (m, 3H), 7.24-7.18 (m, 2H), 4.09 (t, J=17.0, 6.8 Hz, 2H), 3.73-3.70 (m, 1H), 3.08-2.83 (m, 2H), 2.03 (m, 2H), 1.59-1.57 (m, 4H), 1.51 (s, 2H). 1.23 (s, 9H).

### Step-5: 12,12,13,13,14,14,15,15,15-Nonafluoropentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of 12,12,13,13,14,14,15,15,15-nonafluoropentadecyl L-phenylalaninate (7.33 g, 13.64 mmol), triethylamine (1.901 mL, 13.64 mmol) in DCM (48 mL) was added phenyl phosphorodichloridate (2.88 g, 13.64 mmol) in DCM (1 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (2.0 g, 6.82 mmol) in THF (24 mL)/pyridine (12 mL) was added tert-butylmagnesium chloride (13.64 mL, 13.64 mmol) by dropwise under an atmosphere of nitrogen, then the mixture was stirred at room temperature for 30 min. The first prepared mixture was added dropwise to the second prepared mixture at room temperature and stirred rt for 2 h. LCMS indicated completion of reaction. The reaction mixture was filtered and the filtrate was concentrated. Then, the residue was diluted with DCM, washed with water, 0.5 N HCl (three times), brine, dried over Na2SO4, filtered and concentrated. The residue was purified by silica gel chromatography (0-5% MeOH/DCM) to give 12,12,13,13,14,14,15,15,15-nonafluoropentadecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1 g, 1.032 mmol, 15.13% yield). LCMS (M+H)=969.7; Retention time (0.1% TFA)=2.15 min. The diastereomers were separated with Prep-SFC (Instrument: SFC-80 (Thar, Waters), Column: AS 20×250 mm, 10 μm (Daicel), Column temperature: 40° C., Mobile phase: CO₂/MeOH (0.2% ammonia/methanol)=70/30, Flow rate: 70 g/min, Back pressure: 100 bar, Detection wavelength: 260 nm, Cycle time: 6.5 min, Sample solution: 510 mg dissolved in 12 mL methanol, Injection volume: 2 ml) to give first eluting isomer (Example 44A, RT1: 1.66 min, 100.5 mg, 0.127 mmol, 98.59%, yield: 16.79%) as an off-white solid; LCMS (M+H)=969.7; Retention time (0.1% TFA)=2.76 min. HPLC: Retention time (10 mMM NH₄HCO₃)=7.911 min. ¹H NMR (400 MHz, DMSO) δ 8.24 (d, J=8.9 Hz, 1H), 7.85 (s, 2H), 7.35-7.07 (m, 8H), 7.02 (d, J=8.4 Hz, 2H), 6.23 (dd, J=7.5, 4.9 Hz, 1H), 6.14-6.00 (m, 1H), 5.74 (d, J=5.6 Hz, 1H), 4.52 (dd, J=12.6, 7.2 Hz, 1H), 3.89 (dddd, J=42.6, 35.9, 14.2, 9.5 Hz, 5H), 3.63 (d, J=8.9 Hz, 1H), 2.99-2.85 (m, 1H), 2.84-2.63 (m, 2H), 2.18 (dd, J=18.2, 10.0 Hz, 2H), 1.50 (dd, J=15.5, 7.8 Hz, 2H), 1.38-1.28 (m, 4H), 1.18 (d, J=15.8 Hz, 13H) and second eluting isomer (Example 44B, RT2: 2.77 min, 85.5 mg, 0.107 mmol, 97.28%, yield: 14.10%) as an off-white solid; LCMS (M+H)=969.7; Retention time (0.1% TFA)=2.75 min. HPLC: Retention time (10 mMM NH₄HCO₃)=7.876 min. ¹H NMR (400 MHz, DMSO) δ 8.20 (s, 1H), 7.85 (s, 2H), 7.29-7.16 (m, 5H), 7.11 (dd, J=7.2, 3.2 Hz, 3H), 6.98 (d, J=8.5 Hz, 2H), 6.28 (dd, J=7.2, 5.2 Hz, 1H), 6.20-6.06 (m, 1H), 5.78 (d, J=5.5 Hz, 1H), 4.54 (dd, J=12.5, 6.8 Hz, 1H), 4.11-3.92 (m, 2H), 3.95-3.79 (m, 3H), 3.66 (s, 1H), 2.88 (d, J=7.1 Hz, 1H), 2.80 (dd, J=13.5, 7.9 Hz, 1H), 2.75-2.64 (m, 1H), 2.19 (s, 2H), 1.56-1.42 (m, 2H), 1.35 (d, J=6.8 Hz, 4H), 1.27-1.07 (m, 13H).

### Example 45

### 7,7,8,8,9,9,10,10,11,11,12,12,12-Tridecafluorododecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

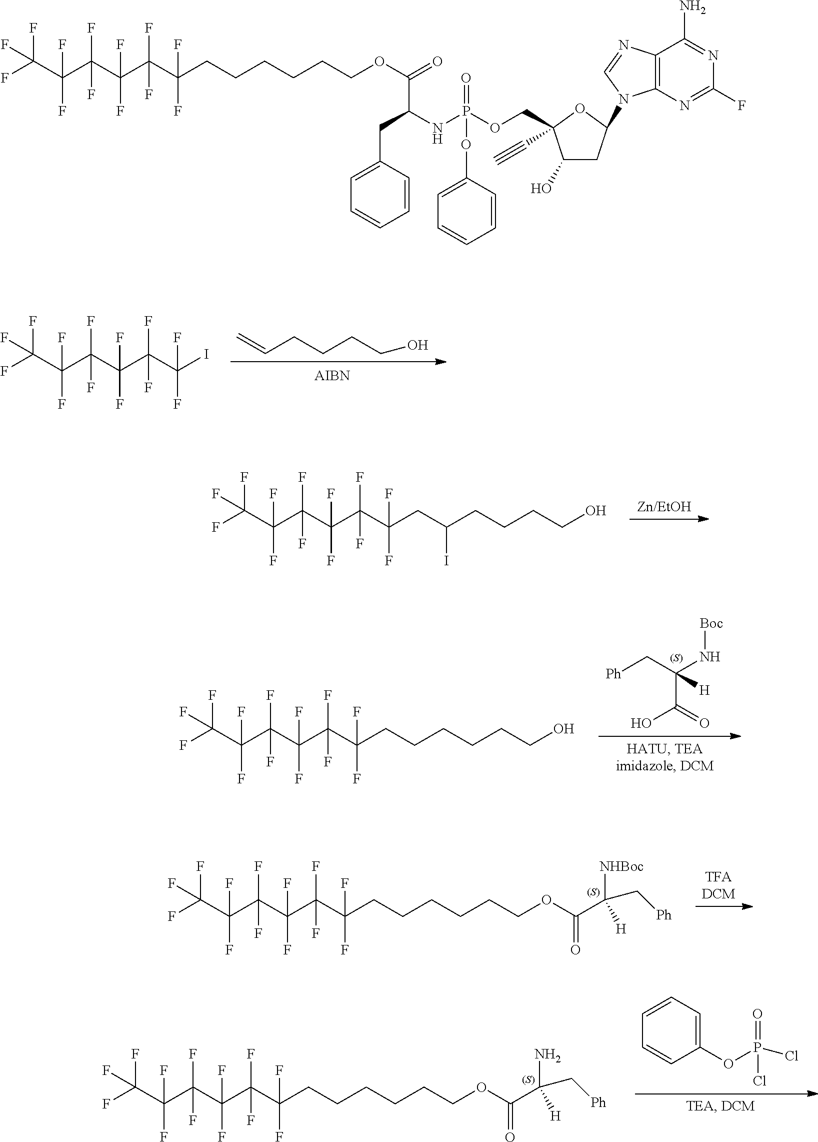

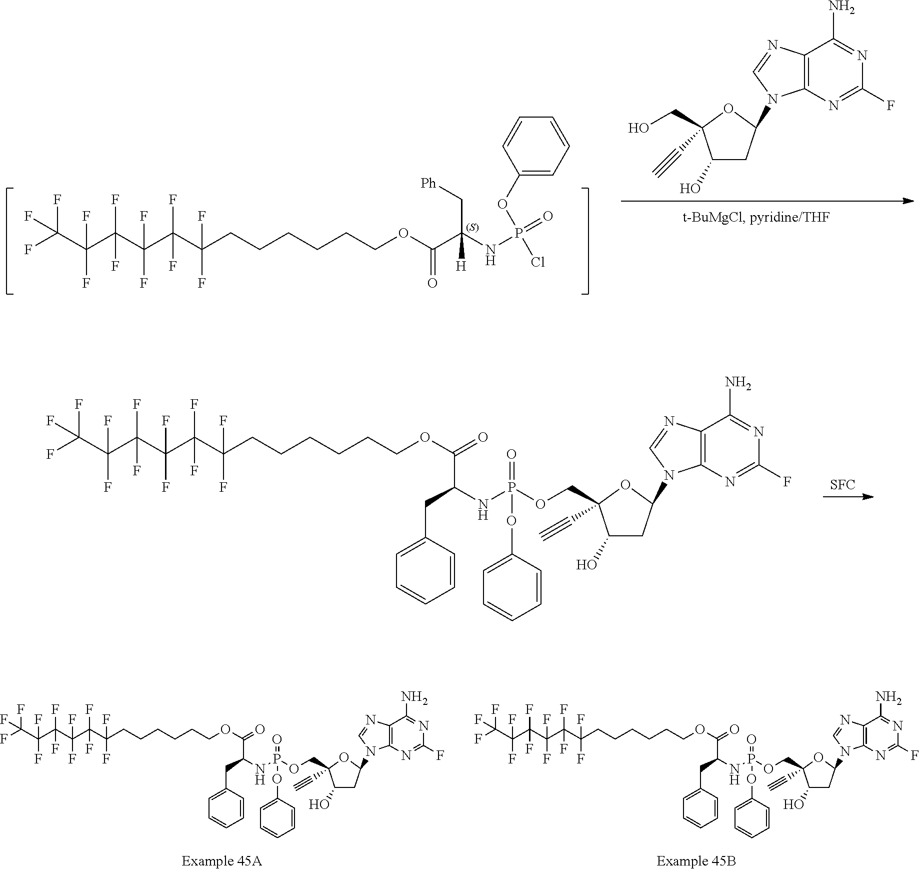

### Step-1: 7,7,8,8,9,9,10,10,11,11,12,12,12-Tridecafluoro-5-iodododecan-1-ol

A mixture of 1,1,1,2,2,3,3,4,4,5,5,6,6-tridecafluoro-6-iodohexane (50 g, 112 mmol), hex-5-en-1-ol (11.23 g, 112 mmol) and AIBN (1.841 g, 11.21 mmol) was heated at 65° C. in a sealed glass tube filled with nitrogen gas for 12 h while gentle stirring. TLC showed presence of new compound. The crude reaction mixture was purified by silica gel chromatography to afford 7,7,8,8,9,9,10,10,11,11,12,12,12-tridecafluoro-5-iodododecan-1-ol (56 g, 103 mmol, 91% yield). ¹H NMR (400 MHz, CDCl₃): δ 4.37-4.33 (m, 1H), 3.66-3.41 (m, 2H), 3.41 (br, 1H), 2.96-2.78 (m, 2H), 1.89-1.83 (m, 2H), 1.67-1.50 (m, 4H).

### Step-2: 7,7,8,8,9,9,10,10,11,11,12,12,12-Tridecafluorododecan-1-ol

To a slurry of zinc (23.94 g, 366 mmol) in ethanol (50 mL) was added 7,7,8,8,9,9,10,10,11,11,12,12,12-tridecafluoro-5-iodododecan-1-ol (40 g, 73.2 mmol). The reaction mixture was heated at 50° C. for 4 h. TLC showed presence of new compound. The reaction was concentrated to dryness. The crude product was purified by silica gel chromatography to afford 7,7,8,8,9,9,10,10,11,11,12,12,12-tridecafluorododecan-1-ol (30 g, 71.4 mmol, 97% yield). ¹H NMR (400 MHz, CDCl₃): δ 3.64 (t, J=16.0, 6.4 Hz, 2H), 2.08-2.03 (m, 2H), 1.64-1.59 (m, 4H), 1.43-1.40 (m, 4H).

### Step-3: 7,7,8,8,9,9,10,10,11,11,12,12,12-Tridecafluorododecyl (tert-butoxycarbonyl)-L-phenylalaninate

A mixture of 7,7,8,8,9,9,10,10,11,11,12,12,12-tridecafluorododecan-1-ol (10 g, 23.80 mmol), (tert-butoxycarbonyl)-L-phenylalanine (8 g, 30.2 mmol), imidazole (6.16 g, 90 mmol), DIPEA (15.80 mL, 90 mmol) and HATU (17.20 g, 45.2 mmol) in DCM (500 mL) was stirred at 25° C. for 16 h. TLC showed presence of new compound. The reaction was diluted with water (500 mL), organic layer was separated and aqueous layer extracted with DCM (250 mL×3). The combined organic phases were washed with brine, dried over Na₂SO₄ and concentrated. The residue was purified by chromatography (pet. ether:EtOAc=1:1) to afford 7,7,8,8,9,9,10,10,11,11,12,12,12-tridecafluorododecyl (tert-butoxycarbonyl)-L-phenylalaninate (12 g, 17.98 mmol, 59.6% yield) as white solid. ¹H NMR (400 MHz, CDCl₃): 7.30-7.23 (m, 3H), 7.15-7.13 (m, 2H), 5.03-4.90 (m, 1H), 4.61-4.56 (m, 1H), 4.11-4.06 (m, 2H), 3.08-3.07 (m, 2H), 2.08-2.02 (m, 2H), 1.60-1.55 (m, 4H), 1.42 (s, 9H), 1.37-1.26 (m, 4H).

Step-4: 7,7,8,8,9,9,10,10,11,11,12,12,12-Tridecafluorododecyl L-phenylalaninate A mixture of 7,7,8,8,9,9,10,10,11,11,12,12,12-tridecafluorododecyl (tert-butoxycarbonyl)-L-phenylalaninate (12 g, 17.98 mmol) and TFA (100 mL, 1298 mmol) in DCM (100 mL) was stirred at 25° C. for 2 h. TLC showed presence of new compound. The reaction mixture was concentrated to dryness. The pH of the residue was adjusted to 7 with aqueous Na₂CO₃ solution and extracted with DCM (300 mL). The organic phase was dried, filtered and concentrated. The residue was purified by chromatography (DCM:MeOH=30:1) to give 7,7,8,8,9,9,10,10,11,11,12,12,12-tridecafluorododecyl L-phenylalaninate (8 g, 14.10 mmol, 78% yield) as white solid. ¹H NMR (400 MHz, CDCl₃): 7.25-7.21 (m, 3H), 7.17-7.14 (m, 2H), 3.97-3.90 (m, 2H), 3.57-3.53 (m, 1H), 2.82-2.79 (m, 2H), 2.52-2.51 (m, 1H), 2.13-1.99 (m, 2H), 1.48-1.44 (m, 4H), 1.30-1.16 (m, 4H).

### Step-5: 7,7,8,8,9,9,10,10,11,11,12,12,12-Tridecafluorododecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of 7,7,8,8,9,9,10,10,11,11,12,12,12-tridecafluorododecyl L-phenylalaninate (5804 mg, 10.23 mmol), triethylamine (3.56 mL, 25.6 mmol) in DCM (60 mL) was added phenyl phosphorodichloridate (5396 mg, 25.6 mmol) in DCM (60 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl (hydroxymethyl)tetrahydrofuran-3-ol (3000 mg, 10.23 mmol) in THF (60 mL)/pyridine (30 mL) was added tert-butylmagnesium chloride (25.6 mL, 25.6 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. NH₄Cl solution was added to residue to adjust pH to 7, organic layer separated and aqueous layer extracted with DCM (300 mL). The combined organic layers were washed with 0.5 N HCl (100 ml) three times and brine, dried over Na₂SO₄, filtered and concentrated. The residue was purified by silica gel chromatography (0-5% MeOH/DCM) to give 7,7,8,8,9,9,10,10,11,11,12,12,12-tridecafluorododecyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (462 mg, 0.463 mmol, 4.52% yield). LCMS (M+H)=999.0; Retention time (0.1% TFA)=2.03 min. The diastereomers were separated with Prep-SFC (Instrument: SFC-80 (Thar, Waters); Column: IG 20×250 mm, 10 μm (Daicel); Column temperature: 40° C.; Mobile phase: CO2/IPA(0.2% ammonia/methanol)=55/45; Flow rate: 80 g/min; Back pressure: 100 bar; Detection wavelength: 260 nm; Cycle time: 6 min) to afford first eluting isomer (Example 45A, 1.46 min, 134.2 mg, 0.134 mmol, 100%, yield: 16.79%) as an off-white solid; LCMS (M+H)=999.7; Retention time (0.1% TFA)=2.60 min. HPLC: Retention time (0.05% TFA)=12.070 min. ¹HNMR (400 MHz, DMSO) δ 8.20 (s, 1H), 8.00-7.71 (m, 2H), 7.32-6.84 (m, 10H), 6.35-6.22 (m, 1H), 6.21-6.05 (m, 1H), 5.87-5.74 (m, 1H), 4.62-4.43 (m, 1H), 4.08-3.95 (m, 2H), 3.95-3.78 (m, 3H), 3.66 (s, 1H), 2.95-2.61 (m, 3H), 2.30-2.00 (m, 2H), 1.50-1.02 (m, 9H) and second eluting isomer (Example 45B, RT2: 2.15 min, 155.1 mg, 0.155 mmol, 100%, yield: 14.10%) as an off-white solid; LCMS (M+H)=999.7; Retention time (0.1% TFA)=2.60 min. HPLC: Retention time (0.05% TFA)=12.048 min. ¹H NMR (400 MHz, DMSO) δ 8.20 (s, 1H), 8.00-7.74 (m, 2H), 7.32-6.71 (m, 10H), 6.28 (dd, J=7.4, 5.1 Hz, 1H), 6.14 (dd, J=13.0, 10.4 Hz, 1H), 5.79 (d, J=5.5 Hz, 1H), 4.55 (d, J=5.7 Hz, 1H), 4.09-3.96 (m, 2H), 3.92-3.81 (m, 3H), 3.66 (s, 1H), 2.78 (ddd, J=50.3, 17.6, 6.9 Hz, 3H), 2.15 (d, J=8.1 Hz, 2H), 1.58-1.02 (m, 9H).

### Example 46

### 5,5,6,6,7,7,8,8,8-Nonafluorooctyl ((((2R,3S,5R)-5-(6-amino-2-fluoro-9H-purin-9-yl) ethynyl-3-hydroxytetrahydrofuran-2-yl)methoxy)(phenoxy)phosphoryl)-L-phenylalaninate

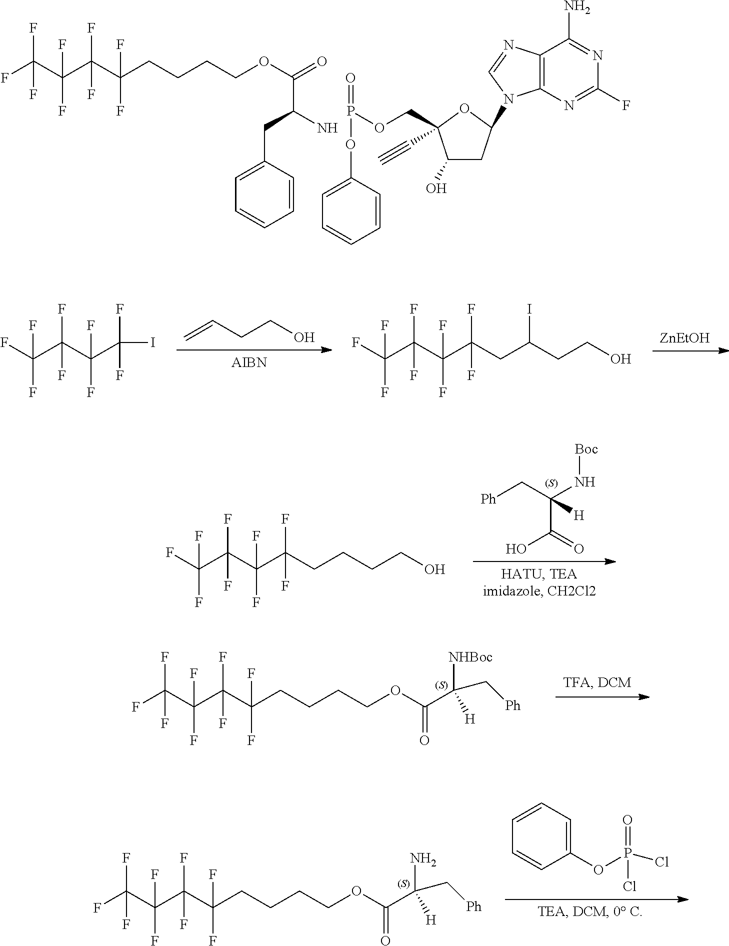

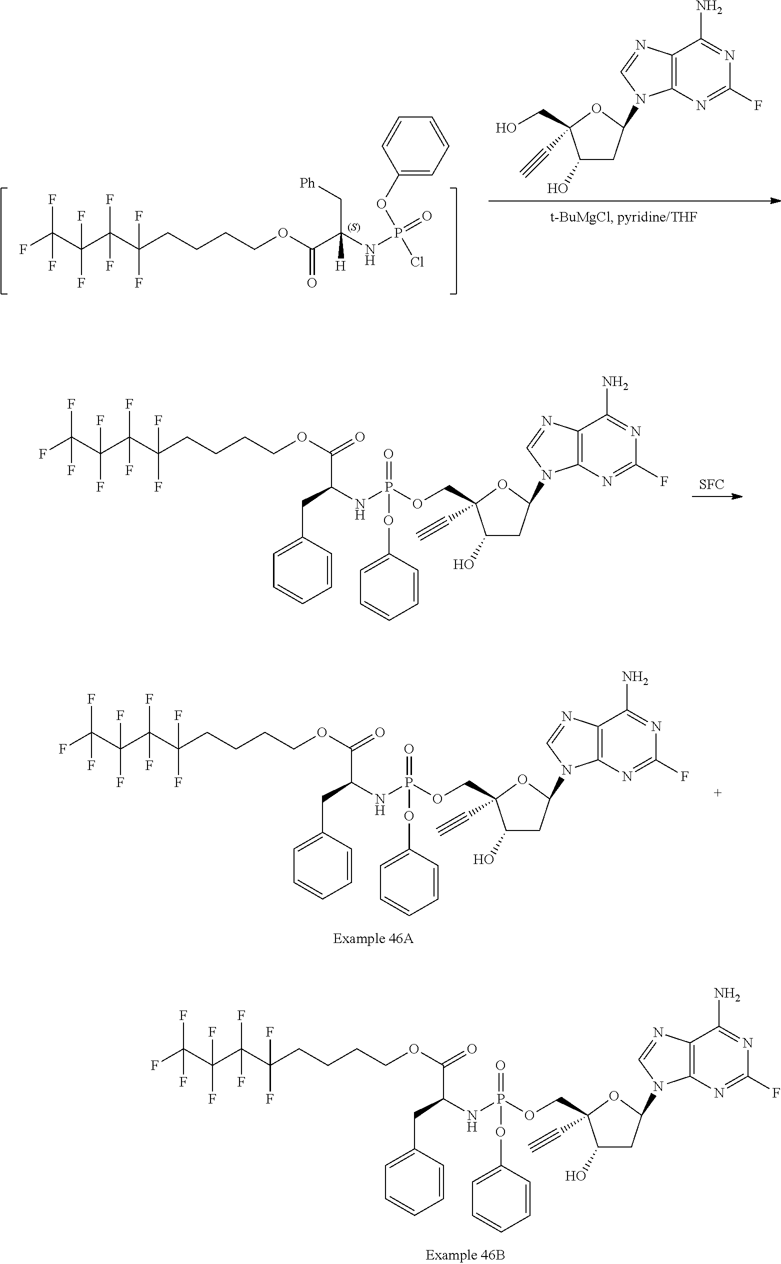

### Step 1: 5,5,6,6,7,7,8,8,8-Nonafluoro-3-iodooctan-1-ol

A mixture of 1,1,1,2,2,3,3,4,4-nonafluoro-4-iodobutane (40 g, 116 mmol), but-3-en-1-ol (8.34 g, 116 mmol) and AIBN (1.899 g, 11.56 mmol) was heated at 65° C. in a sealed glass tube filled with nitrogen gas for 12 h while gentle stirring. The crude reaction mixture was purified by silica gel chromatography to afford 5,5,6,6,7,7,8,8,8-nonafluoro-3-iodooctan-1-ol (32 g, 77 mmol, 66.2% yield). ¹H NMR (400 MHz, CDCl₃) δ 4.53-4.51 (m, 1H), 4.13-4.09 (m, 1H), 3.88-3.76 (m, 2H), 2.92-2.91 (m, 2H), 1.27-1.23 (m, 2H).

### Step 2: 5,5,6,6,7,7,8,8,8-Nonafluorooctan-1-ol

To a slurry of zinc (6.26 g, 96 mmol) in ethanol (5 mL) and was added 5,5,6,6,7,7,8,8,8-nonafluoro-3-iodooctan-1-ol (10.0 g, 23.92 mmol). The reaction mixture was heated at 50° C. for 4 h. TLC showed presence of new compound. The reaction mixture was filtered and the filtrate was concentrated to give the residue. The residue was purified by silica gel chromatography (pet. ether:EtOAc=1:1) to afford 5,5,6,6,7,7,8,8,8-nonafluorooctan-1-ol (3 g, 9.76 mmol, 40.8% yield). ¹H NMR (400 MHz, CDCl₃) δ 3.69-3.66 (m, 2H), 2.37 (s, 1H), 2.15-2.06 (m, 2H), 1.73-1.63 (m, 4H).

### Step 3: 5,5,6,6,7,7,8,8,8-Nonafluorooctyl (tert-butoxycarbonyl)-L-phenylalaninate

A mixture of 5,5,6,6,7,7,8,8,8-nonafluorooctan-1-ol (63.9 g, 219 mmol), (tert-butoxycarbonyl)-L-phenylalanine (58 g, 219 mmol), imidazole (44.6 g, 656 mmol), DIPEA (115 mL, 656 mmol) and HATU (125 g, 328 mmol) in DCM (500 mL) was stirred at 25° C. for 16 h. TLC showed presence of new compound. The reaction mixture was diluted with water (500 mL), organic layer was separated and aqueous layer extracted with DCM (250 mL×3). The combined organic phases were washed with brine, dried over Na₂SO₄ and concentrated. The residue was purified by chromatography (pet. ether:EtOAc=1:1) to afford 5,5,6,6,7,7,8,8,8-nonafluorooctyl (tert-butoxycarbonyl)-L-phenylalaninate (48 g, 82 mmol, 37.4% yield) as white solid. ¹H NMR (400 MHz, CDCl₃) δ 7.30-7.23 (m, 3H), 7.23-7.13 (m, 2H), 5.02-5.00 (m, 1H), 4.57-4.55 (m, 1H), 4.14-4.08 (m, 2H), 3.07-3.06 (m, 2H), 1.66-1.62 (m, 2H), 1.42 (s, 9H), 1.27-1.23 (M, 2H).

### Step 4: 5,5,6,6,7,7,8,8,8-Nonafluorooctyl L-phenylalaninate

To a cold (ice-water bath) solution of 5,5,6,6,7,7,8,8,8-nonafluorooctyl (tert-butoxycarbonyl)-L-phenylalaninate (48 g, 89 mmol) in DCM (500 mL) was added TFA (68.6 mL, 890 mmol) and the mixture was stirred at 25° C. for 2 h. TLC showed presence of new compound. The reaction mixture was concentrated to dryness. The pH of the residue was adjusted to 7 with Na₂CO₃ solution and extracted with DCM (300 mL). The organic phase was dried, filtered and concentrated. The residue was purified by chromatography (DCM:MeOH=20:1) to afford the 5,5,6,6,7,7,8,8,8-nonafluorooctyl L-phenylalaninate (36 g, 78 mmol, 87% yield) as white solid. ¹H NMR (400 MHz, CDCl₃) 7.31-7.22 (m, 3H), 7.19-7.17 (m, 2H), 4.12-4.09 (m, 2H), 3.74-3.70 (m, 2H), 3.06-2.85 (m, 2H), 2.05 (m, 2H), 1.67-1.52 (m, 4H).

### Step 5: 5,5,6,6,7,7,8,8,8-Nonafluorooctyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a cold (ice-water bath) solution of 5,5,6,6,7,7,8,8,8-nonafluorooctyl L-phenylalaninate (5.99 g, 13.63 mmol), triethylamine (1.900 mL, 13.63 mmol) in DCM (60 mL) was added phenyl phosphorodichloridate (2.88 g, 13.63 mmol) in DCM (60 mL) dropwise under an atmosphere of nitrogen, then the reaction mixture was stirred at room temperature for 1 h. To a cold (ice-water bath) solution of (2R,3S,5R)-5-(6-amino-2-fluoro-9H-purin-9-yl)-2-ethynyl-2-(hydroxymethyl)tetrahydrofuran-3-ol (1.997 g, 6.81 mmol) in THF) (40 mL)/pyridine (20 mL) was added tert-butylmagnesium chloride (13.63 mL, 13.63 mmol) dropwise under an atmosphere of nitrogen, then the mixture was stirred at room temperature for 30 min. The first prepared mixture was added dropwise to the second prepared mixture at room temperature and stirred for 2 h. LCMS indicated completion of reaction. The reaction mixture was filtered and the filtration was concentrated. The residue was diluted with DCM, washed with water, 0.5 N HCl (three times), brine, dried over Na2SO4, filtered and concentrated. The residue was purified by silica gel column (0-5% MeOH/DCM) to give 5,5,6,6,7,7,8,8,8-nonafluorooctyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (1.2 g, 1.378 mmol, 20.24% yield). LCMS (M+H)=871.7; Retention time (0.1% TFA)=1.85 min. The diastereomers were separated with Prep-SFC (Instrument: SFC-80 (Thar, Waters), Column: OZ 20×250 mm, 10 μm (Daicel), Column temperature: 40° C., Mobile phase: CO2/MEOH(0.2% ammonia/methanol)=70/30, Flow rate: 80 g/min, Back pressure: 100 bar, Detection wavelength: 260 nm, Cycle time: 6 min) to afford first eluting isomer (Example 46A, RT1: 2.07 min, 100.0 mg, 0.114 mmol, 98.71%, yield: 16.79%) as an off-white solid; LCMS (M+H)=871.7; Retention time (0.1% TFA)=1.85 min. HPLC: Retention time (0.05% TFA)=10.761 min. ¹H NMR (400 MHz, DMSO) δ 8.20 (s, 1H), 8.00-7.74 (m, 2H), 7.32-6.71 (m, 10H), 6.28 (dd, J=7.4, 5.1 Hz, 1H), 6.14 (dd, J=13.0, 10.4 Hz, 1H), 5.79 (d, J=5.5 Hz, 1H), 4.55 (d, J=5.7 Hz, 1H), 4.09-3.96 (m, 2H), 3.92-3.81 (m, 3H), 3.66 (s, 1H), 2.78 (ddd, J=50.3, 17.6, 6.9 Hz, 3H), 2.15 (d, J=8.1 Hz, 2H), 1.58-1.02 (m, 9H) and second eluting isomer (Example 46B, RT2: 2.96 min, 148.0 mg, 0.169 mmol, 98.1%, yield: 14.10%) as an off-white solid; LCMS (M+H)=871.7; Retention time (0.1% TFA)=2.35 min. ¹H NMR (400 MHz, DMSO). HPLC: Retention time (0.05% TFA)=10.769 min. δ 8.20 (s, 1H), 8.00-7.74 (m, 2H), 7.32-6.71 (m, 10H), 6.28 (dd, J=7.4, 5.1 Hz, 1H), 6.14 (dd, J=13.0, 10.4 Hz, 1H), 5.79 (d, J=5.5 Hz, 1H), 4.55 (d, J=5.7 Hz, 1H), 4.09-3.96 (m, 2H), 3.92-3.81 (m, 3H), 3.66 (s, 1H), 2.78 (ddd, J=50.3, 17.6, 6.9 Hz, 3H), 2.15 (d, J=8.1 Hz, 2H), 1.58-1.02 (m, 9H).

### Example 47

Isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

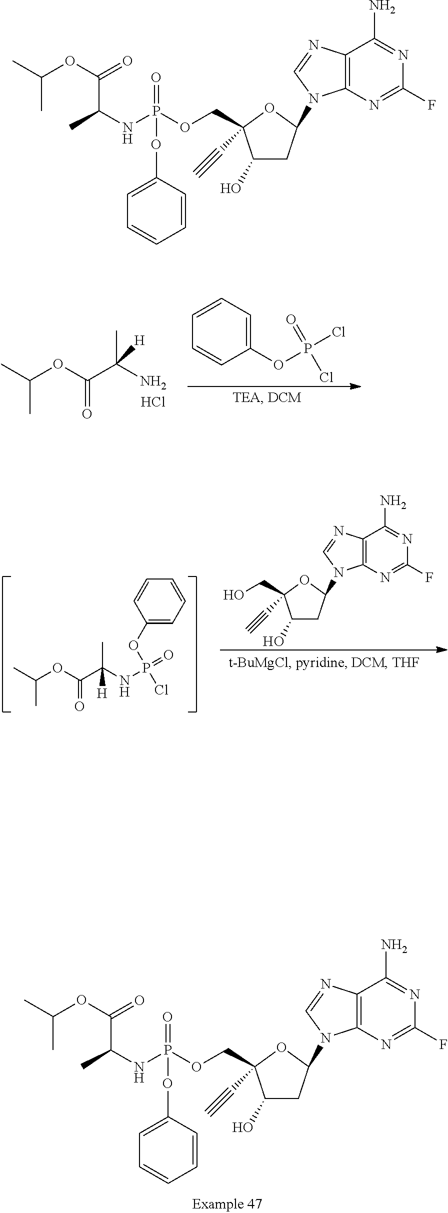

### Isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a cold (ice-water bath) mixture of isopropyl isopropyl L-alaninate hydrochloride (114 mg, 0.682 mmol) and triethylamine (0.190 mL, 1.364 mmol) in DCM (6 mL) was added phenyl phosphorodichloridate (144 mg, 0.682 mmol) in DCM (1 mL) dropwise, then the reaction mixture was stirred at 0° C. for 1 h. To a solution of (2R,3S,5R)-5-(6-amino-2-fluoro-9H-purin-9-yl)-2-ethynyl-2-(hydroxymethyl)tetrahydrofuran-3-ol (100 mg, 0.341 mmol) in THF (4 mL) and pyridine (2 mL) was added tert-butylmagnesium chloride (0.852 mL, 0.852 mmol) dropwise under an atmosphere of nitrogen in 0° C., then the mixture was stirred at the same temperature for 30 min. The first prepared mixture was added dropwise to the second prepared mixture at 0° C. and stirred room temperature for 2 h. LCMS showed the reaction was completed. The reaction mixture was filtered and the filtrate was concentrated, then the residue was diluted with EtOAc (50 mL). The organic layer was washed with water (20 mL), brine (20 mL), dried over Na₂SO₄, filtered and concentrated. The residue was purified by reverse phase chromatography (SepaFlash® C18, 0-100% CH₃CN/10 mM NH4HCO₃ H₂O) to give isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (10 mg, 0.018 mmol, 5.21% yield) as a white solid. LCMS (M+H)=562.8; Retention time (10 mMM NH₄HCO₃)=1.28, 1.29 min. HPLC Retention time (10 mMM NH₄HCO₃)=7.20, 7.34 min. ¹H NMR (400 MHz, CDCl₃) δ 7.93 (d, J=38.8 Hz, 1H), 7.32-7.28 (m, 1H), 7.25-7.22 (m, 1H), 7.21-7.16 (m, 2H), 7.12 (dd, J=16.4, 8.0 Hz, 1H), 6.63 (brs, 2H), 6.34 (dt, J=11.6, 6.8 Hz, 1H), 5.02-4.92 (m, 1H), 4.81 (dt, J=6.8, 4.0 Hz, 1H), 4.45-4.33 (m, 2H), 4.20 (ddd, J=79.6, 11.6, 9.6 Hz, 1H), 4.00-3.89 (m, 1H), 2.78-2.64 (m, 3H), 1.32 (dd, J=10.8, 7.2 Hz, 3H), 1.21-1.13 (m, 6H).

### Example 48

### 2-Ethylbutyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

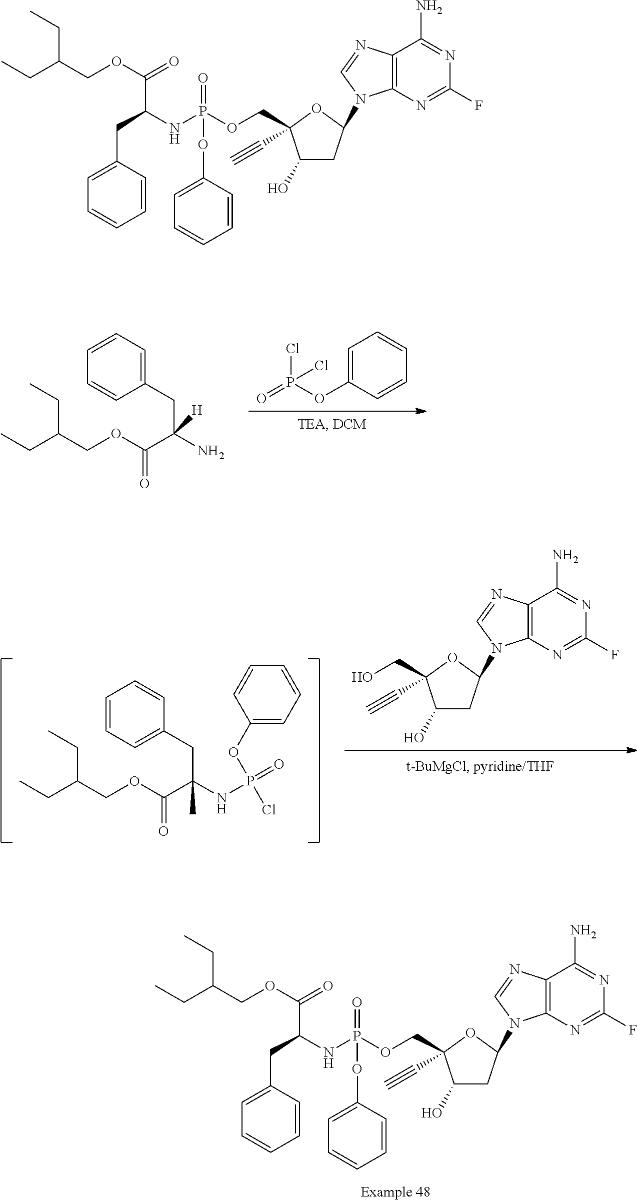

### 2-Ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a solution of 2-ethylbutyl L-phenylalaninate (213 mg, 0.852 mmol), triethylamine (0.119 mL, 0.852 mmol) in DCM (5 mL) was added phenyl phosphorodichloridate (0.127 mL, 0.852 mmol) in DCM (5 mL) dropwise under an atmosphere of nitrogen at 0° C., then the reaction mixture was stirred at 0° C. for 1 h. To a solution of (2R,3S,5R)-5-(6-amino-2-fluoro-9H-purin-9-yl)-2-ethynyl-2-(hydroxymethyl)tetrahydrofuran-3-ol (100 mg, 0.341 mmol) in THF (20 mL) and pyridine (10 mL) was added tert-butylmagnesium chloride (0.852 mL, 0.852 mmol) dropwise under an atmosphere of nitrogen at 0° C. Then the mixture was stirred at the same temperature for 30 min. The first prepared mixture was added to the second prepared mixture by dropwise at 0° C. and stirred at 10° C. for 2 h and at room temperature for 2 h. LCMS showed the reaction was completed. The reaction mixture was filtered and the filtrate was concentrated, then the residue was diluted with EtOAc (50 mL). The organic layer was washed with water (20 mL), brine (20 mL), dried over Na₂SO₄, filtered and concentrated. The residue was purified by reverse phase chromatography (SepaFlash® C18, 0-100% CH₃CN/10 mM NH₄HCO₃ H₂O) to give 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (10 mg, 0.015 mmol, 4.31% yield) as a white solid. LCMS (M+H)=680.8; Retention time (10 mM NH₄HCO₃)=1.59 min. HPLC Retention time (10 mM NH₄HCO₃)=9.54 min. ¹H NMR (400 MHz, CDCl₃) δ 8.19-7.75 (m, 1H), 7.26-7.17 (m, 6H), 7.17-6.99 (m, 5H), 6.42-6.20 (m, 2H), 5.01-4.60 (m, 1H), 4.32-4.15 (m, 2H), 4.11-3.75 (m, 4H), 3.18-2.90 (m, 2H), 2.75-2.50 (m, 3H), 1.54-1.37 (m, 1H), 1.34-1.18 (m, 4H), 10.90-0.76 (m, 6H).

### Example 49

### 2-Ethylbutyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-alaninate

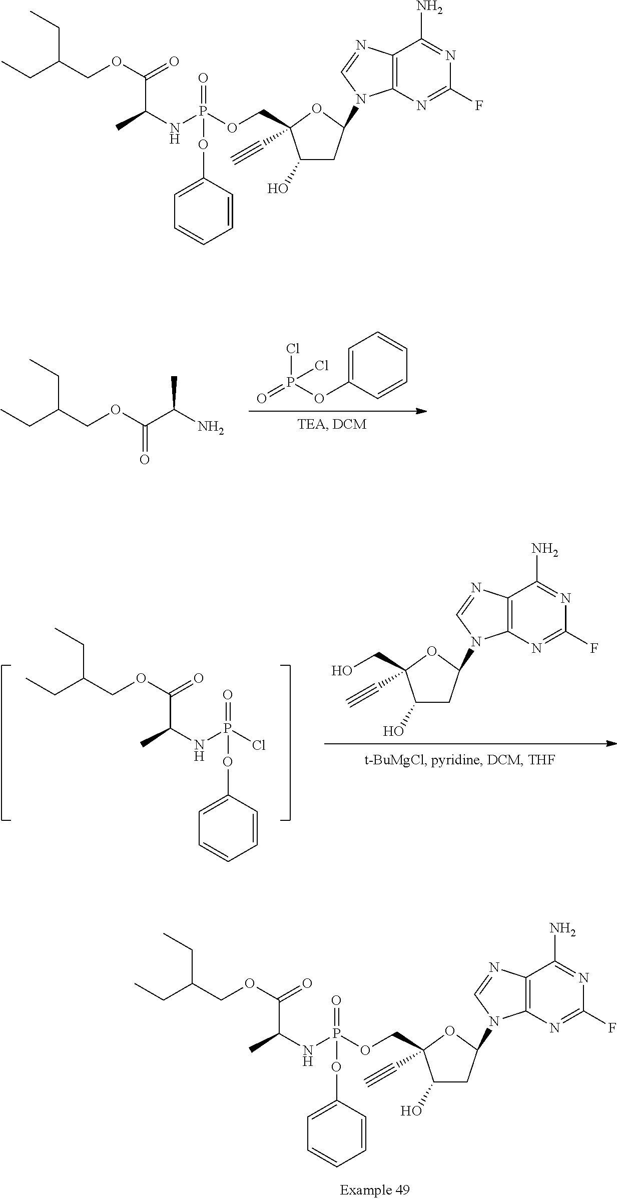

### 2-Ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate

To a mixture of 2-ethylbutyl L-alaninate (118 mg, 0.682 mmol) and triethylamine (69.0 mg, 0.682 mmol) in DCM (5 mL) was added phenyl phosphorodichloridate (144 mg, 0.682 mmol) dropwise at 0° C., then the reaction mixture was stirred at 0° C. for 1 h. To a solution of (2R,3S,5R)-5-(6-amino-2-fluoro-9H-purin-9-yl)-2-ethynyl-2-(hydroxymethyl)tetrahydrofuran-3-ol (100 mg, 0.341 mmol) in THF (4.00 mL) and pyridine (2 mL) was added tert-butylmagnesium chloride (100 mg, 0.852 mmol) dropwise under an atmosphere of nitrogen at 0° C. Then, the mixture was stirred at the same temperature for 30 min. The first prepared mixture was added to the second prepared mixture by dropwise at 0° C. and stirred at 0° C. for 2 h. LCMS showed the reaction was completed. The reaction mixture was filtered and the filtrate was concentrated The residue was diluted with EtOAc (50 mL), washed with water (20 mL), brine (20 mL), dried over Na₂SO₄, filtered and concentrated. The residue was purified by reverse phase chromatography (SepaFlash® C18, 0-100% CH₃CN/10 mMM NH₄HCO₃ H₂O) to give 2-ethylbutyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-alaninate (10 mg, 0.016 mmol, 4.69% yield) as a white solid. LCMS (M+H)=605.2; Retention time (0.05% TFA)=1.83 min. HPLC Retention time (10 mM NH₄HCO₃)=8.67 min. ¹H NMR (400 MHz, CDCl₃) δ 8.04-7.87 (m, 1H), 7.32-7.28 (m, 2H), 7.22-7.10 (m, 3H), 6.58 (brs, 2H), 6.39-6.32 (m, 1H), 5.00-4.72 (m, 1H), 4.47-4.20 (m, 3H), 4.18-3.92 (m, 4H), 2.80-2.63 (m, 3H), 1.51-1.43 (m, 1H), 1.37-1.27 (m, 6H), 0.90-0.80 (m, 6H).

### Example 50

### Isopropyl ((((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

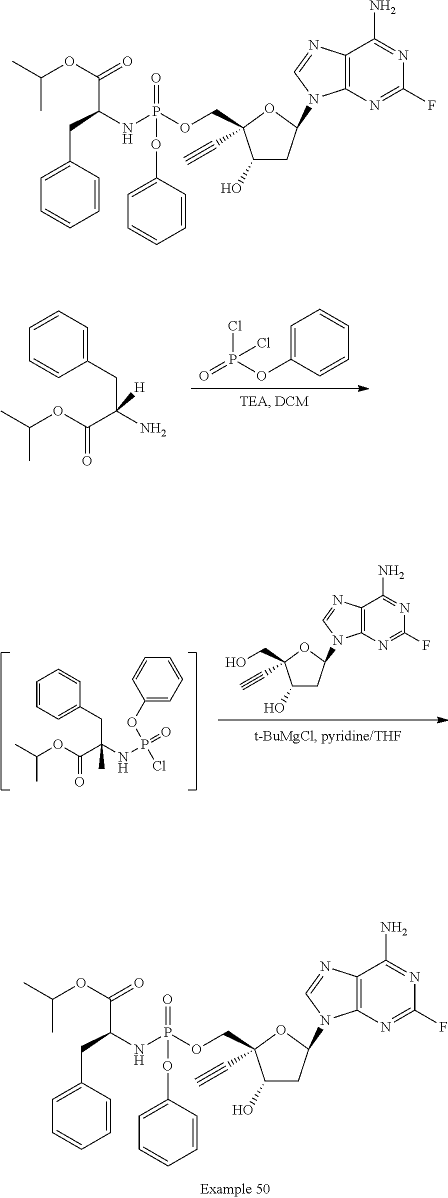

### Isopropyl ((((2R,3S,5R)-5-(6-amino-2-fluoro-9H-purin-9-yl)-2-ethynyl-3-hydroxytetrahydrofuran-2-yl)methoxy) (phenoxy)phosphoryl)-L-phenylalaninate

To a solution of isopropyl L-phenylalaninate (212 mg, 1.023 mmol), triethylamine (0.143 mL, 1.023 mmol) in DCM (6 mL) was added phenyl phosphorodichloridate (0.153 mL, 1.023 mmol) in DCM (1 mL) dropwise under an atmosphere of nitrogen at about 0° C., then the reaction mixture was stirred at room temperature for 1 h. To a solution of (2R,3S,5R)-5-(6-amino-2-fluoro-9H-purin-9-yl)-2-ethynyl-2-(hydroxymethyl)tetrahydrofuran-3-ol (120 mg, 0.409 mmol) in THF (3 mL) and Pyridine (1.5 mL) was added tert-butylmagnesium chloride (1.023 mL, 1.023 mmol) dropwise under an atmosphere of nitrogen at 0° C. Then the mixture was stirred at the same temperature for 30 min. The first prepared mixture was added dropwise to the second prepared mixture at 0° C. and stirred at 10° C. for 2 h. LCMS indicated completion of reaction. The reaction mixture was quenched with water, partitioned between dichloromethane (100 mL) and water 100 (mL). The organic phase was washed with water (100 mL) and brine (100 mL), dried over sodium sulphate and evaporated in vacuo to give the crude product. The residue was purified by reverse phase chromatography (SepaFlash® C18, 0-100% CH₃CN/10 mM NH₄HCO₃ H₂O) to give isopropyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (30 mg, 0.047 mmol, 11.48% yield) as a white solid. LCMS (M+H)=638.9; Retention time (10 mM NH₄HCO₃)=1.644 min. HPLC: Retention time (10 mM NH₄HCO₃)=8.351 min. ¹H NMR (400 MHz, CDCl₃) δ 7.99-7.75 (m, 1H), 7.31-7.27 (m, 1H), 7.25-7.14 (m, 5H), 7.09 (dd, J=18.6, 7.3 Hz, 4H), 6.34-6.28 (m, 1H), 6.03 (s, 2H), 4.95 (dt, J=12.2, 6.3 Hz, 1H), 4.73 (tt, J=32.8, 7.8 Hz, 1H), 4.31-4.05 (m, 3H), 4.00-3.74 (m, 1H), 3.67 (s, 1H), 2.97 (dd, J=10.8, 6.7 Hz, 2H), 2.80-2.55 (m, 3H), 1.20-1.11 (m, 6H).

### Example 51

### Nonadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

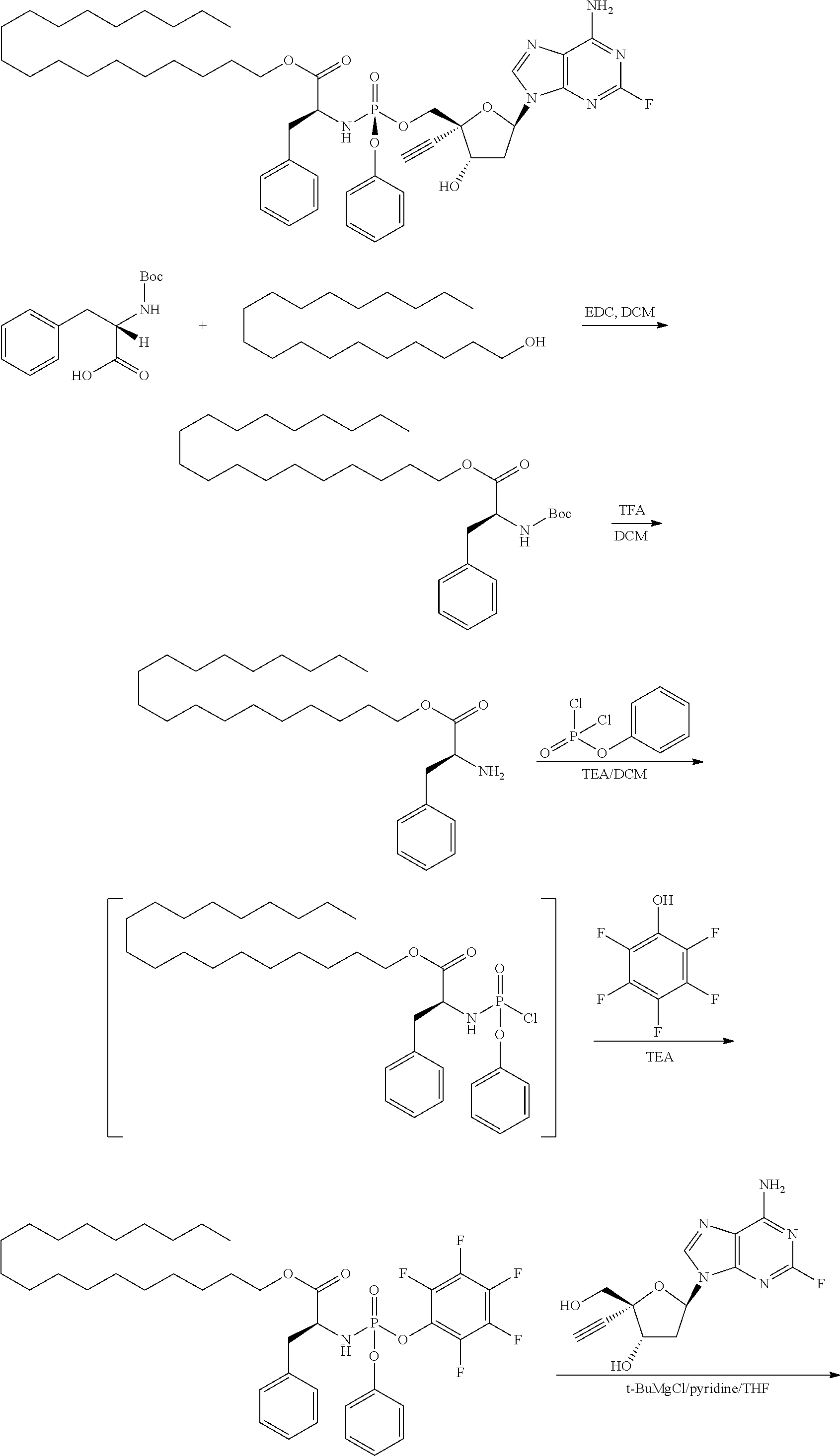

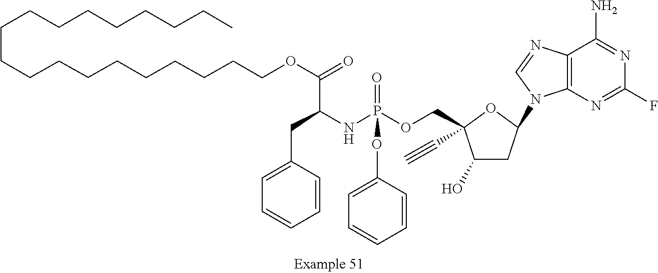 [SMILES: C#C[C@]1(CO[P@@](=O)(N[C@@H](Cc2ccccc2)C(=O)OCCCCCCCCCCCCCCCCCCC)Oc2ccccc2)O[C@@H](n2cnc3c(N)nc(F)nc32)C[C@@H]1O]

### Step 1: Nonadecyl (tert-butoxycarbonyl)-L-phenylalaninate

To a stirred mixture of (tert-butoxycarbonyl)-L-phesnylalanine (26.6 g, 100 mmol) and EDC (24.96 g, 130 mmol) in DCM (150 mL) was added DMAP (1.224 g, 10.02 mmol) at 0° C. After 30 min, nonadecan-1-ol (28.5 g, 100 mmol) was added and the resulting reaction mixture was stirred at 25° C. for 16 h. TLC showed the reaction was completed. The reaction mixture was diluted with water (80 mL) and extracted with DCM (100 mL×2). The combined organic phases were washed with brine, dried over Na₂SO₄ and concentrated. The residue was purified by silica gel chromatography (pet. ether:EtOAc=10:1) to afford nonadecyl (tert-butoxycarbonyl)-L-phenylalaninate (42 g, 79 mmol, 79 yield) as a yellow oil. ¹H NMR (400 MHz, CDCl₃) δ 7.30-7.23 (m, 3H), 7.14-7.12 (m, 2H), 4.99-4.97 (m, 1H), 4.57-4.55 (m, 1H), 4.09-4.05 (m, 2H), 3.09-3.06 (t, J=6.8 Hz, 3H), 1.61-1.55 (t, J=6.8 Hz, 3H), 1.41 (s, 9H), 1.29 (bs, 30H), 0.86 (t, J=6.8 Hz, 3H).

### Step 2: Nonadecyl L-phenylalaninate

To a stirred solution of nonadecyl (tert-butoxycarbonyl)-L-phenylalaninate (42.5 g, 80 mmol) in DCM (300 mL) was added TFA (60 mL, 779 mmol) at 0° C. After 5 min, the cold bath was removed and the mixture was stirred at 25° C. for 2 h. TLC showed the reaction was completed. The reaction was concentrated in vacuum and the resulting residue was added water (100 mL) and pH was adjusted to 8-9 with sat. Na₂CO₃ and extracted with DCM (100 mL×3). The combined organic phases were washed with brine (80 mL), dried over Na₂SO₄, filtered and concentrated. The residue was purified by silica gel chromatography (DCM:MeOH=100:0 to 20:1) to afford nonadecyl L-phenylalaninate (31 g, 71.8 mmol, 90% yield). ¹H NMR (400 MHz, CDCl₃) δ 7.30 (t, J=7.2 Hz, 2H), 7.25-7.13 (m, 3H), 4.09 (t, J=6.7 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.59 (d, J=6.8 Hz, 2H), 1.27 (bs, J=9.3 Hz, 32H), 0.88 (t, J=6.8 Hz, 3H).

### Step 3: Nonadecyl ((S)-(perfluorophenoxy)(phenoxy)phosphoryl)-L-phenylalaninate

To a stirred solution of nonadecyl L-phenylalaninate (31 g, 71.8 mmol) in anhydrous DCM (50 mL) was added triethylamine (10.98 mL, 79 mmol) dropwise at −70° C. over 15 min. To this mixture was added a solution of phenyl phosphorodichloridate (15.00 g, 71.1 mmol) in anhydrous DCM (30 mL) over 1 h. The reaction mixture was stirred at this temperature for 30 min and then allowed to warm to 0° C. over 2 h and stirred for 1 h. To this mixture was added a solution of 2,3,4,5,6-pentafluorophenol (13.09 g, 71.1 mmol) and triethylamine (10.98 mL, 79 mmol) in DCM (20 mL) over 20 min. The crude mixture was allowed to stir at 0° C. for 4 h. TLC showed the reaction was completed. The white solid (triethylamine hydrochloride) was filtered off and washed with DCM (50 mL). The filtrate was concentrated under reduced pressure, the resulting residue was triturated with THF (250 mL), and the solids (triethylamine hydrochloride salt) were removed by filtration. The cake was washed with THF (2×50 mL) and the combined filtrates were concentrated under reduced pressure. The crude product was triturated with EtOAc (200 mL) and collected by filtration nonadecyl ((S)-(perfluorophenoxy)(phenoxy)phosphoryl)-L-phenylalaninate (20 g, 21.22 mmol, 29.6% yield) as a white solid (>98% de as determined by ³¹PNMR). ¹H NMR (400 MHz, CDCl₃) δ 7.41-7.27 (m, 2H), 7.25-7.15 (m, 5H), 7.15-6.98 (m, 3H), 4.43 (d, J=5.8 Hz, 1H), 4.13-3.96 (m, 2H), 3.84-3.74 (m, 1H), 3.10 (ddd, J=20.0, 13.7, 5.8 Hz, 2H), 1.54 (s, 2H), 1.36-1.08 (m, 32H), 0.88 (t, J=6.8 Hz, 3H). ³¹PNMR (CDCl₃, 162 MHz) δ −1.54.

### Step 4: Nonadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40 mL) and pyridine (2.000 mL) was added tert-butylmagnesium chloride (1.431 mL, 1.432 mmol) dropwise at 0° C. and stirred at 25° C. for 30 min. A solution of nonadecyl ((S)-(perfluorophenoxy)(phenoxy)phosphoryl)-L-phenylalaninate (514 mg, 0.682 mmol) in THF (5 mL) was added dropwise to the above solution at −15° C. and stirred at −15° C. for 16 h. TLC showed the reaction was completed. The reaction was diluted with 1M HCl (10 mL) and extracted with EtOAc (20 mL×3). The combined organic layers were washed with a solution of NaHCO₃ (20 mL), brine (20 mL), dried with Na₂SO₄, filtered and concentrated. The crude product was purified by silica gel chromatography (40 g, DCM:MeOH=100:0 to 20:1) to afford nonadecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (152 mg, 0.174 mmol, 25.5% yield) as white solid. LCMS (M+H)=862.7; Retention time (0.1% TFA)=3.69 min. ¹H NMR (400 MHz, CDCl₃) δ 7.99 (s, 1H), 7.28 (d, J=5.8 Hz, 1H), 7.25-7.07 (m, 9H), 6.32 (dd, J=7.1, 4.4 Hz, 1H), 6.27-5.78 (m, 2H), 4.71 (t, J=7.6 Hz, 1H), 4.23 (dt, J=20.1, 11.3 Hz, 3H), 4.04 (t, J=6.7 Hz, 2H), 3.89 (s, 1H), 3.75-3.46 (m, 1H), 3.00 (d, J=6.2 Hz, 2H), 2.83-2.62 (m, 3H), 1.53 (s, 2H), 1.24 (d, J=8.0 Hz, 32H), 0.88 (t, J=6.8 Hz, 3H).

### Example 52

### Henicosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

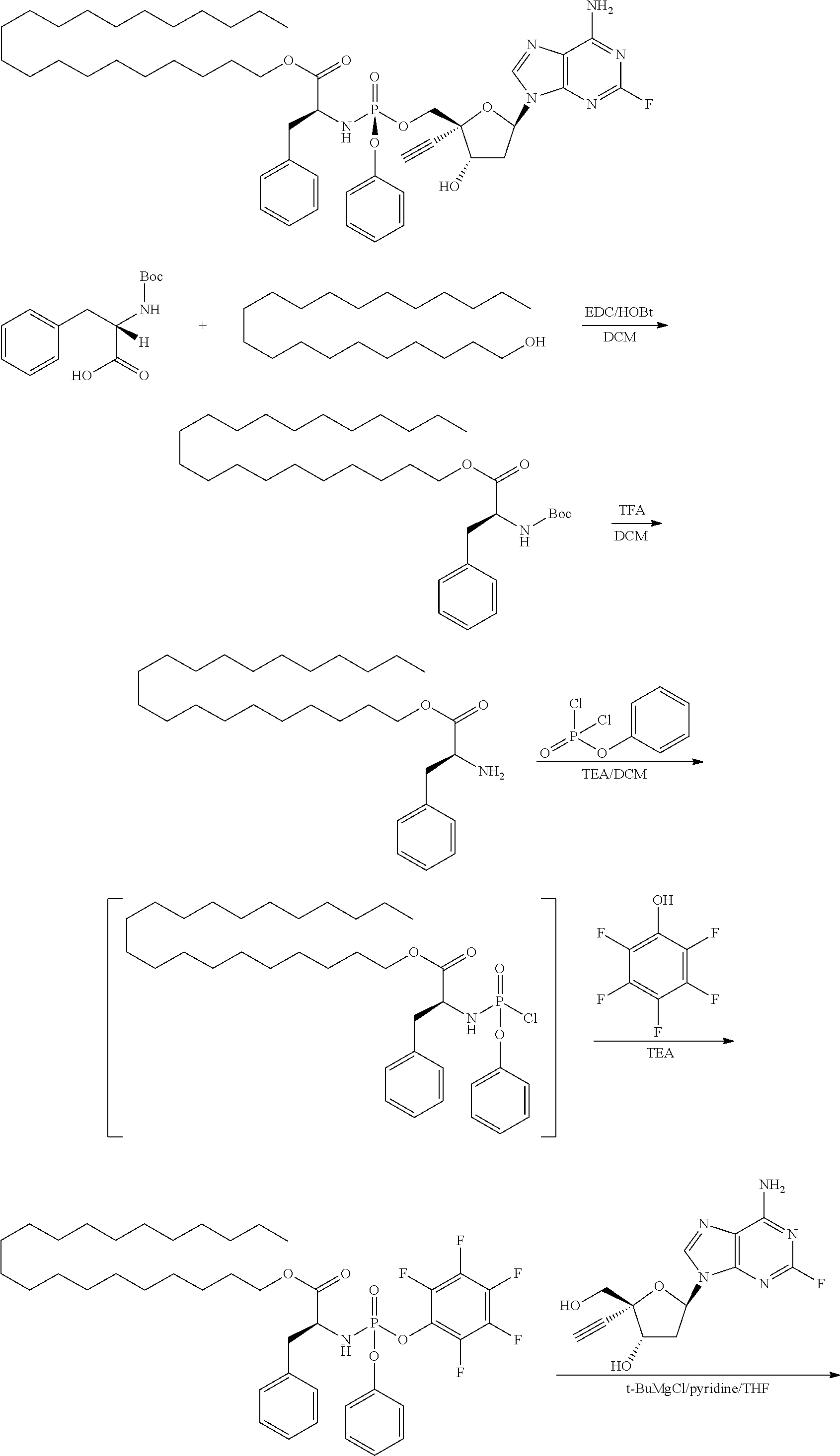

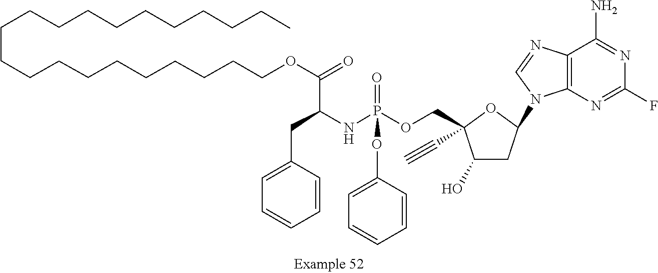 [SMILES: C#C[C@]1(CO[P@@](=O)(N[C@@H](Cc2ccccc2)C(=O)OCCCCCCCCCCCCCCCCCCCCC)Oc2ccccc2)O[C@@H](n2cnc3c(N)nc(F)nc32)C[C@@H]1O]

### Step 1: Henicosyl (tert-butoxycarbonyl)-L-phenylalaninate

To a stirred mixture of (tert-butoxycarbonyl)-L-phenylalanine (5 g, 18.85 mmol), HOBt (3.82 g, 28.3 mmol) and EDC (4.34 g, 22.62 mmol) in DCM (150 mL) was added TEA (2.63 mL, 18.85 mmol) at 0° C. After stirring for 30 min, henicosan-1-ol (5.89 g, 18.85 mmol) was added and the resulting reaction mixture was stirred at 25° C. for 16 h. TLC showed the reaction was completed. The reaction mixture was diluted with water (80 mL) and extracted with DCM (100 mL×2). The combined organic phases were washed with brine, dried over Na₂SO₄, filtered and concentrated. The residue was purified by silica gel chromatography (pet. ether:EtOAc=10:1) to afford henicosyl (tert-butoxycarbonyl)-L-phenylalaninate (6.1 g, 10.90 mmol, 57.8% yield) as a yellow oil. ¹H NMR (400 MHz, CDCl3) δ 7.30-7.22 (m, 3H), 7.14-7.12 (m, 2H), 4.99-4.97 (m, 1H), 4.57-4.55 (m, 1H), 4.09-4.05 (m, 2H), 3.09-3.06 (t, J=6.8 Hz, 3H), 1.61-1.55 (t, J=6.8 Hz, 3H), 1.41 (s, 9H), 1.25 (bs, 32H), 0.85 (t, J=6.8 Hz, 3H).

### Step 2: Henicosyl L-phenylalaninate

To a stirred solution of henicosyl (tert-butoxycarbonyl)-L-phenylalaninate (6.1 g, 10.90 mmol) in DCM (50 mL) was added TFA (25 mL, 324 mmol) at 0° C. After 5 min, the mixture was warmed to 25° C. and stirred for 2 h. TLC showed the presence of new product. The reaction mixture was concentrated, the residue was diluted with water (50 mL) and the pH adjusted to 8-9 with sat. Na₂CO₃ and extracted with DCM (50 mL×3). The combined organic phases were washed with brine (80 mL), dried over Na₂SO₄, filtered and concentrated. The residue was purified by silica gel chromatography (DCM:MeOH=100:0 to 20:1) to afford henicosyl L-phenylalaninate (4.56 g, 9.92 mmol, 91% yield). ¹H NMR (400 MHz, CDCl₃) δ 7.34-7.27 (m, 2H), 7.26-7.16 (m, 3H), 4.09 (dd, J=8.6, 4.8 Hz, 2H), 3.72 (dd, J=7.8, 5.4 Hz, 1H), 3.08 (dd, J=13.5, 5.4 Hz, 1H), 2.87 (dd, J=13.5, 7.8 Hz, 1H), 1.58 (s, 2H), 1.25 (bs, 36H), 0.88 (t, J=6.8 Hz, 3H).

### Step 3: Henicosyl ((S)-(perfluorophenoxy)(phenoxy)phosphoryl)-L-phenylalaninate

To a stirred solution of henicosyl L-phenylalaninate (4.5 g, 9.79 mmol) in anhydrous DCM (50 mL) was added triethylamine (1.497 mL, 10.77 mmol) dropwise at −70° C. over 15 min. To the reaction mixture was added a solution of phenyl phosphorodichloridate (2.044 g, 9.69 mmol) in anhydrous DCM (30 mL) over 1 h. The reaction mixture was stirred for 30 min at −70° C., then allowed to warm to 0° C. over 2 h and stirred for 1 h. To this mixture was added a solution of 2,3,4,5,6-pentafluorophenol (1.784 g, 9.69 mmol) and triethylamine (1.497 mL, 10.77 mmol) in DCM (20 mL) over 20 min. The crude mixture was allowed to stir at 0° C. for 4 h. The white solid (triethylamine hydrochloride) was filtered off and washed with DCM (50 mL). The filtrate was concentrated under reduced pressure and the residue was triturated with tert-buty methyl ether (250 mL). Then, the solid triethylamine hydrochloride salt was removed by filtration. The cake was washed with tert-buty methyl ether (2×50 mL), and the combined filtrates were concentrated under reduced pressure. The crude product was triturated with 20% EtOAc in hexanes (200 mL) and solids collected by filtration to afford henicosyl ((S)-(perfluorophenoxy)(phenoxy)phosphoryl)-L-phenylalaninate (2.2 g, 2.64 mmol, 27.0 yield) as a white solid (>94% de as determined by ³¹PNMR). ¹H NMR (400 MHz, CDCl₃) δ 7.35 (t, J=7.9 Hz, 2H), 7.26-7.14 (m, 6H), 7.05 (dd, J=7.0, 2.4 Hz, 2H), 4.50-4.36 (m, 1H), 4.04 (ddd, J=10.7, 8.7, 4.0 Hz, 2H), 3.87-3.76 (m, 1H), 3.10 (ddd, J=20.0, 13.7, 5.8 Hz, 2H), 1.54 (s, 2H), 1.25 (s, 36H), 0.88 (t, J=6.8 Hz, 3H). ³¹PNMR (CDCl₃, 162 MHz) δ −1.54.

### Step 4: Henicosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.2 g, 0.682 mmol) in THF (40 mL) and pyridine (2.000 mL) was added tert-butylmagnesium chloride (1.432 mL, 1.432 mmol) dropwise at 0° C. and the resulting reaction was stirred at 25° C. for 30 min. A solution of henicosyl ((S)-(perfluorophenoxy)(phenoxy)phosphoryl)-L-phenylalaninate (0.693 g, 0.887 mmol) in THF (20 mL) was added dropwise to the above solution at −15° C. and stirred at −15° C. for 4 h. TLC showed presence of new product. The reaction was quenched with 2M HCl (20 mL) and extracted with EtOAc (30 mL×3). The combined organic phases were washed with a solution of NaHCO₃ (20 mL), brine (20 mL), dried with Na₂SO₄, filtered and concentrated. The crude product was purified by silica gel chromatography (25 g, DCM/MeOH=100/0 to 10/1) to afford henicosyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (139 mg, 0.150 mmol, 21.96% yield) as white solid. LCMS: Retention time (0.5% TFA)=4.127 min. ¹H NMR (400 MHz, CDCl₃) δ 7.93 (s, 1H), 7.26-7.02 (m, 10H), 6.30 (d, J=4.3 Hz, 1H), 6.27-5.94 (m, 2H), 4.72 (s, 1H), 4.23 (dd, J=16.1, 8.5 Hz, 3H), 4.03 (s, 3H), 3.79-3.44 (m, 1H), 2.99 (d, J=5.9 Hz, 2H), 2.71 (d, J=5.7 Hz, 3H), 1.52 (s, 2H), 1.24 (d, J=9.0 Hz, 36H), 0.88 (t, J=6.8 Hz, 3H).

### Example 53

### Pentadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

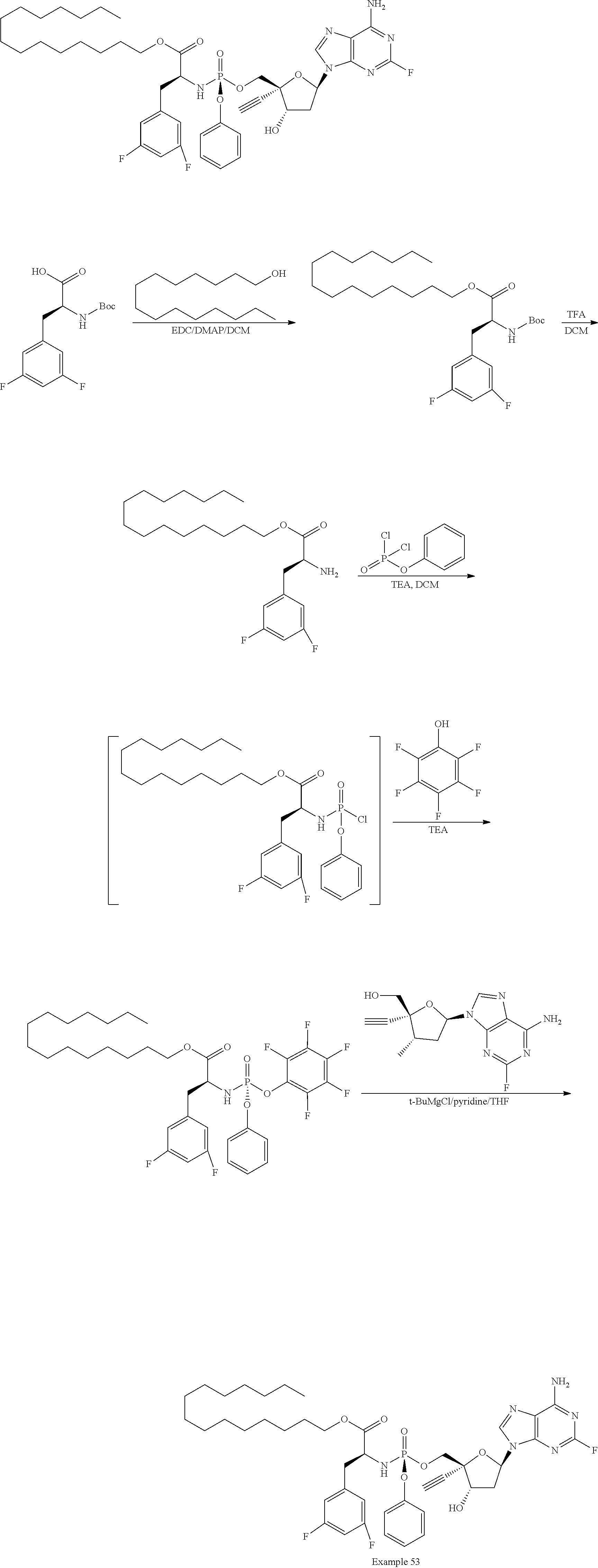

### Step 1: Pentadecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (4 g, 13.28 mmol) in DCM (50 mL) was added DMAP (0.162 g, 1.328 mmol) and EDC (3.31 g, 17.26 mmol) at 0° C. After 30 min, pentadecan-1-ol (3.94 g, 17.26 mmol) was added and the resulting reaction mixture was stirred at 25° C. for 2 h. LCMS showed the reaction was completed. The reaction mixture was diluted with water (40 mL) and extracted with DCM (2×100 mL). The combined organic layers were washed with brine (40 mL), dried over Na₂SO₄, filtered and concentrated to afford pentadecyl (S)-2-((tert-butoxycarbonyl)amino)-3-(3,5-difluorophenyl)propanoate (5.8 g, 11.33 mmol, 85% yield) as a yellow oil which was used in the next step without further purification. LCMS: (M+Na)=534.2; Retention time (0.1% TFA)=3.19 min.

### Step 2: Pentadecyl (S)-2-amino-3-(3,5-difluorophenyl)propanoate

To a stirred solution of pentadecyl (S)-2-((tert-butoxycarbonyl)amino)-3-(3,5-difluorophenyl)propanoate (5.8 g, 11.33 mmol) in DCM (50 mL) was added TFA (15 mL, 195 mmol) at 0° C. The resulting reaction mixture was stirred at 25° C. for 4 h. LCMS showed the reaction was completed. The reaction was concentrated and the residue was diluted with NaHCO₃ (30 mL) and extracted with DCM (3×80 mL). The combined organic layers were washed with brine (50 mL), dried over Na₂SO₄, filtered and concentrated. The residue was purified by silica gel chromatography (petroleum ether/ethyl acetate=2/1) to give pentadecyl (S)-2-amino-3-(3,5-difluorophenyl)propanoate (4.5 g, 10.93 mmol, 96 yield) as a light yellow oil. LCMS (M+H)=412.4; Retention time (0.05% TFA)=2.01 min.

### Step 3: Pentadecyl (S)-3-(3,5-difluorophenyl)-2-(((S)-(perfluorophenoxy)(phenoxy)phosphoryl)amino)propanoate

To a stirred solution of pentadecyl (S)-2-amino-3-(3,5-difluorophenyl)propanoate (4.5 g, 10.93 mmol) in DCM (60 mL) was added triethylamine (1.672 mL, 12.03 mmol) dropwise at −70° C. To this mixture was added a solution of phenyl phosphorodichloridate (2.284 g, 10.82 mmol) in anhydrous DCM (8 mL) dropwise. The reaction mixture was stirred at this temperature for additional 30 min and allowed to warm to 0° C. over 2 h and stirred for 1 h. To this mixture was added a solution of 2,3,4,5,6-pentafluorophenol (2.012 g, 10.93 mmol) and triethylamine (1.672 mL, 12.03 mmol) in DCM (10 mL) dropwise. The mixture was allowed to stir at 0° C. overnight. LCMS showed the reaction was completed. The white solid (triethylamine hydrochloride) was filtered off and washed with DCM (1×10 mL). The filtrate was concentrated under reduced pressure, the residue was triturated with tert-butyl mether ether (100 mL) and the solid triethylamine hydrochloride salt was removed by filtration. The cake was washed with tert-butyl mether ether (2×15 mL) and the combined filtrates were concentrated under reduced pressure. The residue was triturated with 15% EtOAc in hexanes (80 mL) and solids collected by filtration to give pentadecyl (S)-3-(3,5-difluorophenyl)-2-(((S)-(perfluorophenoxy)(phenoxy)phosphoryl)amino)propanoate (1.3 g, 1.772 mmol, 16.21% yield) as a white solid (>98% de as determined by ³¹PNMR). LCMS (M+H)=734; Retention time (0.1% TFA)=3.82 min. ³¹PNMR (CDCl₃, 162 MHz) δ −1.61.

### Step 4: Pentadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40.00 mL) and pyridine (10 mL) was added tert-butylmagnesium chloride (1.4 mL, 1.364 mmol) dropwise at −15° C. The reaction mixture was stirred at the same temperature for 1 h. Then, to this reaction mixture was added a solution of pentadecyl (S)-3-(3,5-difluorophenyl)-2-(((S)-(perfluorophenoxy)(phenoxy)phosphoryl)amino)propanoate (701 mg, 0.955 mmol) in anhydrous THF (15 mL) dropwise and stirred at −15° C. for 4 h. LCMS showed the reaction was completed. The reaction mixture was quenched with 2N NH₄Cl (20 mL) and diluted with EtOAc (200 mL). The organic layer was washed with water (40 mL), brine (40 mL), dried over Na₂SO₄, filtered and concentrated. The residue was purified by reverse phase chromatography (SepaFlash® C18 column, BOSTON, 40 g, 0-100% CH₃CN/10 mM NH₄HCO₃ H₂O) to give pentadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (214 mg, 0.246 mmol, 36.0% yield) as a white solid. LCMS (M+H)=843.0; Retention time (10 mM NH₄HCO₃)=2.87 min; purity: 100% (254 nm). HPLC Retention time (10 mM NH₄HCO₃)=9.31 min. ¹H NMR (400 MHz, CDCl₃) δ 7.94 (s, 1H), 7.30-7.24 (m, 2H), 7.16-7.09 (m, 3H), 6.66-6.57 (m, 3H), 6.42-6.10 (m, 3H), 4.79 (dd, J=12.4, 7.2 Hz, 1H), 4.40-4.26 (m, 3H), 4.25-4.17 (m, 1H), 4.01 (t, J=6.0 Hz, 2H), 3.91 (d, J=4.8 Hz, 1H), 2.95 (d, J=6.0 Hz, 2H), 2.80 (ddd, J=13.6, 6.8, 4.0 Hz, 1H), 2.73 (s, 1H), 2.72-2.66 (m, 1H), 1.51 (quint, J=6.8 Hz, 2H), 1.28-1.19 (m, 24H), 0.88 (t, J=6.8 Hz, 3H).

### Example 54

### Undecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

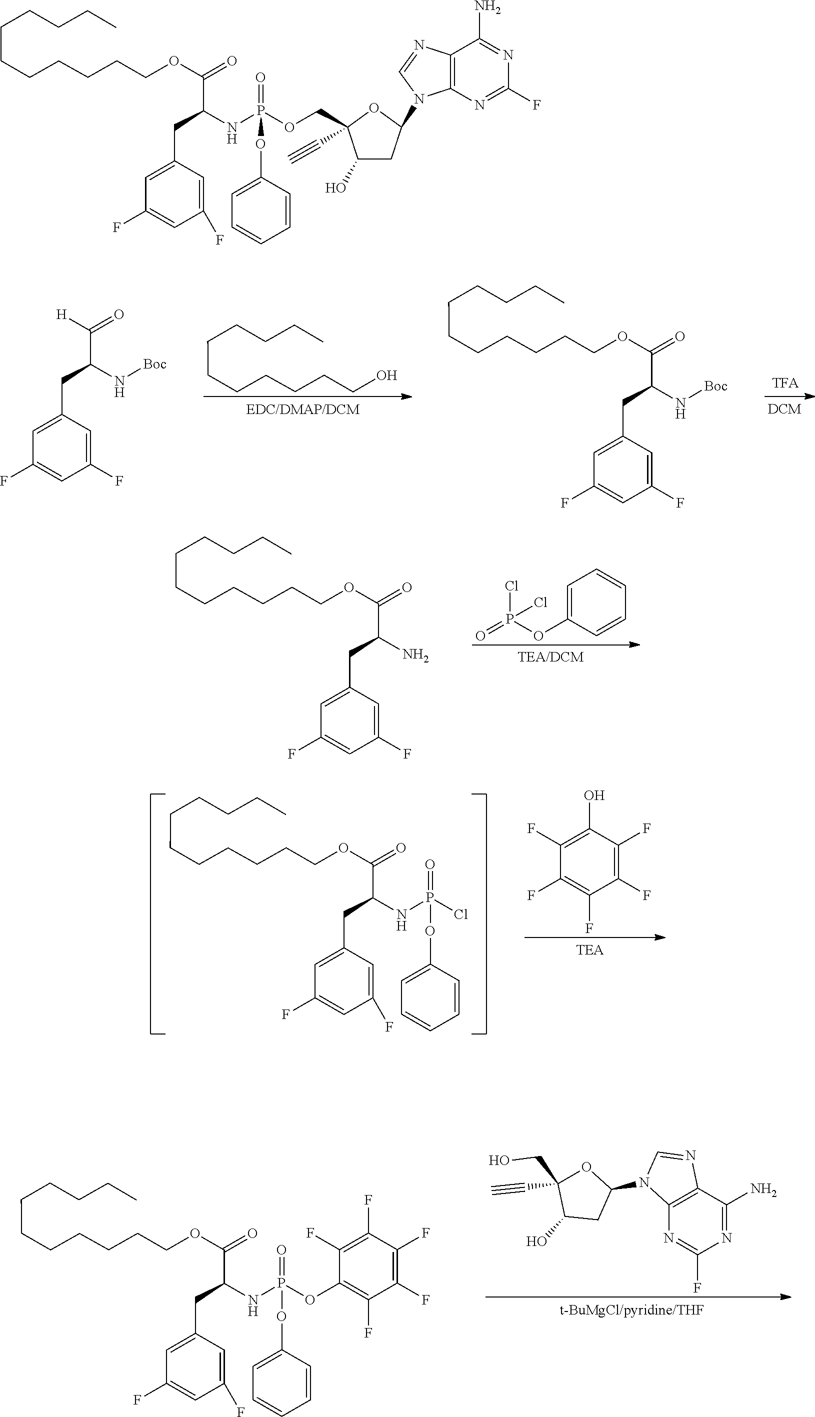

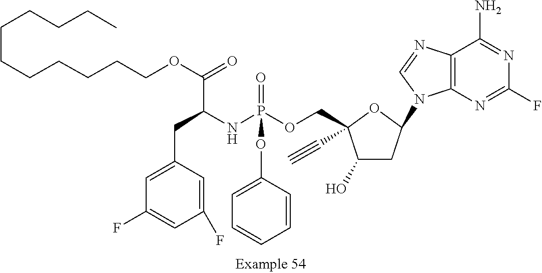 [SMILES: C#C[C@]1(CO[P@@](=O)(N[C@@H](Cc2cc(F)cc(F)c2)C(=O)OCCCCCCCCCCC)Oc2ccccc2)O[C@@H](n2cnc3c(N)nc(F)nc32)C[C@@H]1O]

### Step 1: Undecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (5 g, 16.60 mmol) in DCM (80 mL) was added DMAP (0.203 g, 1.660 mmol) and EDC (4.14 g, 21.57 mmol) at 0° C. After for 30 min, undecan-1-ol (3.72 g, 21.57 mmol) was added at 0° C. and the resulting reaction mixture was stirred at 25° C. for 4 h. LCMS showed the reaction was completed. The reaction mixture was diluted with water (50 mL) and extracted with DCM (2×100 mL). The combined organic layers were washed with brine (40 mL), dried over Na₂SO₄, filtered and concentrated to afford undecyl (S)-2-((tert-butoxycarbonyl)amino)-3-(3,5-difluorophenyl)propanoate (6.9 g, 15.15 mmol, 91% yield) as a yellow solid which was used in the next step without further purification. LCMS: (M+Na)=478.1; Retention time (0.1% TFA)=2.80 min.

### Step 2: Undecyl (S)-2-amino-3-(3,5-difluorophenyl)propanoate

To a stirred solution of undecyl (S)-2-((tert-butoxycarbonyl)amino)-3-(3,5-difluorophenyl)propanoate (6.9 g, 15.15 mmol) in DCM (60 mL) was added TFA (15 mL, 195 mmol) at 0° C. The resulting reaction mixture was stirred at 25° C. for 4 h. LCMS showed the reaction was completed. The reaction mixture was concentrated under reduced pressure and the residue was diluted with NaHCO₃ (20 mL)/water (50 mL) and extracted with DCM (3×100 mL). The combined organic layers were washed with brine (60 mL), dried over Na₂SO₄, filtered and concentrated. The residue was purified by silica gel chromatography (petroleum ether/ethyl acetate=2/1) to give undecyl (S)-2-amino-3-(3,5-difluorophenyl)propanoate (3.8 g, 10.52 mmol, 69.4% yield) as a yellow solid. LCMS (M+H)=356.3; Retention time (0.05% TFA)=1.78 min.

### Step 3: Undecyl (5)-3-(3,5-difluorophenyl)-2-((S)-(perfluorophenoxy)(phenoxy)phosphor-yl)amino)propanoate

To a stirred solution of undecyl (S)-2-amino-3-(3,5-difluorophenyl)propanoate (3.8 g, 10.69 mmol) in DCM (80 mL) was added triethylamine (1.635 mL, 11.76 mmol) dropwise at −70° C. To this mixture was added a solution of phenyl phosphorodichloridate (2.233 g, 10.58 mmol) in anhydrous DCM (10 mL) dropwise. The reaction mixture was stirred at this temperature for additional 30 min and allowed to warm to 0° C. over 2 h and stirred for 1 h. To this mixture was added a solution of 2,3,4,5,6-pentafluorophenol (1.968 g, 10.69 mmol) and triethylamine (1.635 mL, 11.76 mmol) in DCM (20 mL) dropwise. The resulting mixture was allowed to stir at 0° C. for 4 h. LCMS showed the reaction was completed. The white solid (triethylamine hydrochloride) was filtered off and washed with DCM (1×10 mL). The filtrate was concentrated under reduced pressure and the residue was triturated with tert-butyl mether ether (150 mL) and the solid triethylamine hydrochloride salt was removed by filtration. The cake was washed with tert-butyl mether ether (2×30 mL) and the filtrate was concentrated under reduced pressure. The residue was triturated with 15% EtOAc in hexanes (80 mL) and solids collected by filtration undecyl (S)-3-(3,5-difluorophenyl)-2-(((S)-(perfluorophenoxy)(phenoxy)phosphoryl)amino)propanoate (2.0 g, 2.95 mmol, 27.6% yield) as a white solid (>98% de as determined by ³¹PNMR). LCMS (M+H)=678.2; Retention time (0.05% TFA)=3.13 min. ³¹PNMR (CDCl₃, 162 MHz) δ −1.58.

### Step 4: Undecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40.00 mL) and pyridine (10 mL) was added tert-butylmagnesium chloride (1.4 mL, 1.364 mmol) dropwise at −15° C. After 1 h, a solution of undecyl (S)-3-(3,5-difluorophenyl)-2-(((S)-(perfluorophenoxy)(phenoxy)phosphoryl)amino)propanoate (647 mg, 0.955 mmol) in anhydrous THF (15 mL) was added dropwise and stirred at −15° C. for 4 h. LCMS showed the reaction was completed. The reaction mixture was quenched with 2N NH₄C₁ (10 mL) and the mixture was diluted with EtOAc (200 mL). The organic layer was washed with water (40 mL), brine (40 mL), dried over Na₂SO₄, filtered and concentrated. The residue was purified by reverse phase chromatography (SepaFlash® C18 column, BOSTON, 40 g, 0-100% CH₃CN/10 mM NH₄HCO₃ H₂O) to give undecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (201 mg, 0.249 mmol, 36.5% yield) as a white solid. LCMS (M+H)=786.9; Retention time (10 mM NH₄HCO₃)=2.32 min; purity: 100% (254 nm). HPLC Retention time (10 mM NH₄HCO₃)=7.69 min. ¹H NMR (400 MHz, CDCl₃) δ 7.92 (s, 1H), 7.31-7.24 (m, 2H), 7.16-7.10 (m, 3H), 6.67-6.58 (m, 3H), 6.32 (dd, J=7.2, 4.0 Hz, 1H), 6.07 (brs, 2H), 4.78 (t, J=7.2 Hz, 1H), 4.32 (ddd, J=26.4, 11.2, 7.2 Hz, 2H), 4.26-4.18 (m, 1H), 4.08 (d, J=10.4 Hz, 1H), 4.03 (t, J=6.8 Hz, 2H), 3.65 (s, 1H), 2.96 (d, J=6.4 Hz, 2H), 2.82 (ddd, J=13.6, 7.2, 4.4 Hz, 1H), 2.73 (s, 1H), 2.72-2.64 (m, 1H), 1.52 (quint, J=6.8 Hz, 2H), 1.28-1.20 (m, 16H), 0.88 (t, J=6.8 Hz, 3H).

### Example 55

### Tridecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

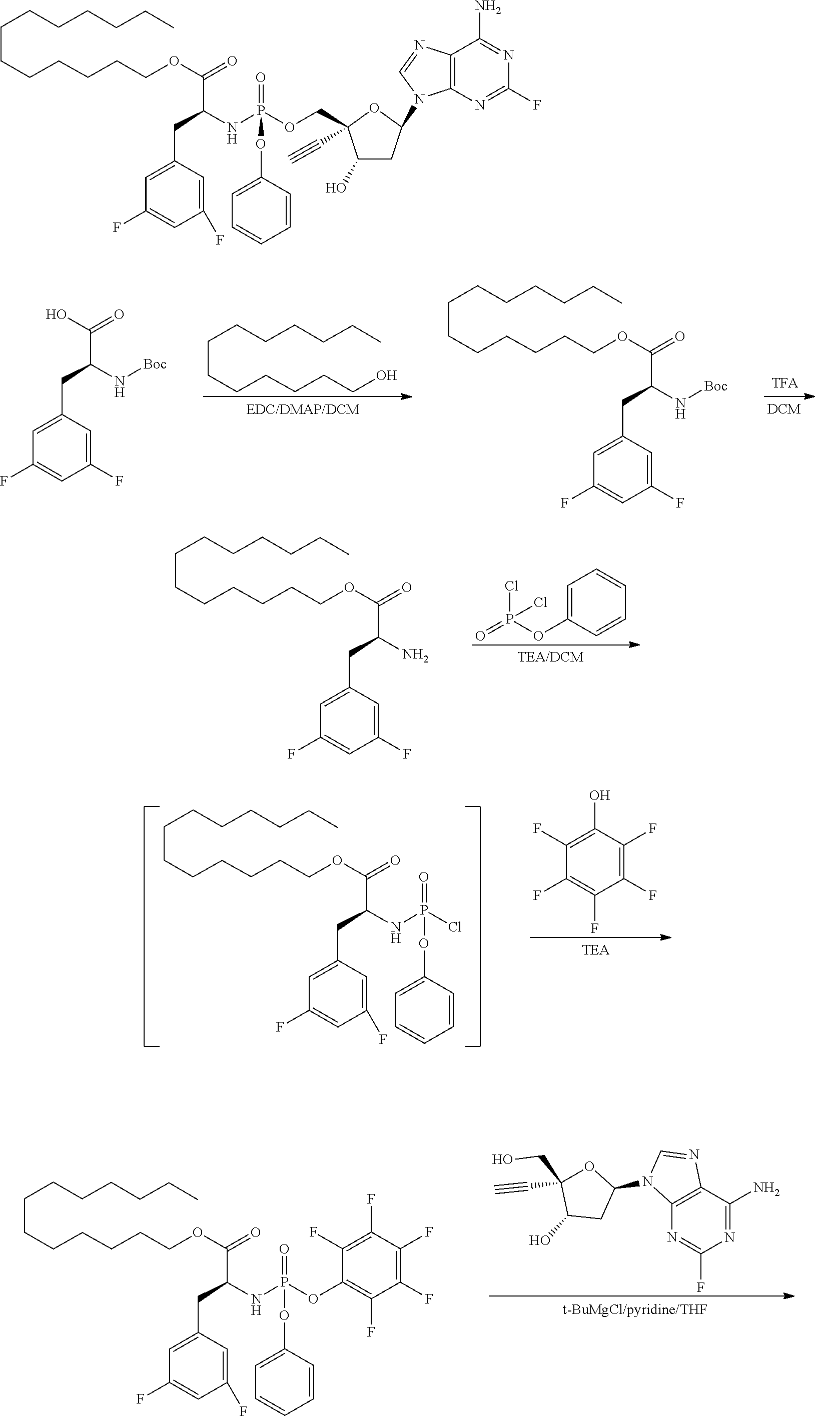

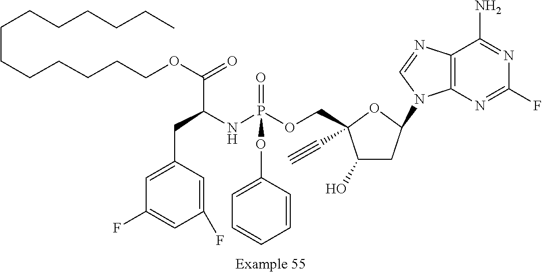 [SMILES: C#C[C@]1(CO[P@@](=O)(N[C@@H](Cc2cc(F)cc(F)c2)C(=O)OCCCCCCCCCCCCC)Oc2ccccc2)O[C@@H](n2cnc3c(N)nc(F)nc32)C[C@@H]1O]

### Step 1: Tridecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (4 g, 13.28 mmol) in DCM (60 mL) was added DMAP (0.162 g, 1.328 mmol) and EDC (3.31 g, 17.26 mmol) at 0° C. After 30 min, tridecan-1-ol (3.46 g, 17.26 mmol) was added and the resulting reaction mixture was stirred at 25° C. for 4 h. LCMS showed the reaction was completed. The reaction mixture was diluted with water (50 mL) and extracted with DCM (2×100 mL). The combined organic phases were washed with brine (40 mL), dried over Na₂SO₄, filtered and concentrated to afford tridecyl (S)-2-((tert-butoxycarbonyl)amino)-3-(3,5-difluorophenyl)propanoate (5.7 g, 11.79 mmol, 89% yield) as a yellow solid which was used in the next step without further purification. LCMS: (M+Na)=506.2; Retention time (0.1% TFA)=3.15 min.

### Step 2: Tridecyl (S)-2-amino-3-(3,5-difluorophenyl)propanoate

To a stirred solution of tridecyl (S)-2-((tert-butoxycarbonyl)amino)-3-(3,5-difluorophenyl)propanoate (5.7 g, 11.79 mmol) in DCM (50 mL) was added TFA (12 mL, 156 mmol) dropwise at 0° C. The resulting reaction mixture was stirred at 25° C. for 4 h. LCMS showed the reaction was completed. The reaction mixture was concentrated under reduced pressure. The residue was diluted with NaHCO₃ (20 mL)/water (50 mL) and extracted with DCM (3×100 mL). The combined organic layers were washed with brine (60 mL), dried over Na₂SO₄, filtered and concentrated. The residue was purified by silica gel chromatography (petroleum ether/ethyl acetate=2/1) to give tridecyl (S)-2-amino-3-(3,5-difluorophenyl)propanoate (2.6 g, 6.56 mmol, 55.7% yield) as a light yellow solid. LCMS (M+H)=384.4; Retention time (0.05% TFA)=1.88 min.

### Step 3: Tridecyl (S)-3-(3,5-difluorophenyl)-2-((S)-(perfluorophenoxy)(phenoxy)phosphor-yl)amino)propanoate

To a stirred solution of tridecyl (S)-2-amino-3-(3,5-difluorophenyl)propanoate (2.6 g, 6.78 mmol) in DCM (60 mL) was added triethylamine (1.037 mL, 7.46 mmol) dropwise at −70° C. To this mixture was added a solution of phenyl phosphorodichloridate (1.416 g, 6.71 mmol) in anhydrous DCM (10 mL) dropwise. The reaction mixture was stirred at this temperature for additional 30 min and then allowed to warm to 0° C. over 2 h and stirred for 1 h. To this mixture was added a solution of 2,3,4,5,6-pentafluorophenol (1.248 g, 6.78 mmol) and triethylamine (1.037 mL, 7.46 mmol) in DCM (15 mL) dropwise. The resulting mixture was allowed to stir at 0° C. for 4 h. LCMS showed the reaction was completed. The white solid (triethylamine hydrochloride) was filtered off and washed with DCM (1×10 mL). The filtrate was concentrated under reduced pressure, the residue was triturated with tert-butyl mether ether (120 mL) and the triethylamine hydrochloride salt was removed by filtration. The cake was washed with tert-butyl mether ether (2×30 mL) and the filtrate was concentrated under reduced pressure. The residue was triturated with 15% EtOAc in hexanes (50 mL) and solids collected by filtration tridecyl (S)-3-(3,5-difluorophenyl) (((S)-(perfluorophenoxy)(phenoxy)phosphoryl)amino)propanoate (1.1 g, 1.559 mmol, 22.99% yield) as a white solid (>98% de as determined by ³¹PNMR). LCMS (M+H)=706.3; Retention time (0.05% TFA)=3.40 min. ³¹P NMR (CDCl₃, 162 MHz) δ −1.58.

### Step 4: Tridecyl (S)-2-(((S)-(((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40.00 mL) and pyridine (10 mL) was added tert-butylmagnesium chloride (1.4 mL, 1.364 mmol) dropwise at −15° C. After 1 h, a solution of tridecyl (S)-3-(3,5-difluorophenyl)-2-(((S)-(perfluorophenoxy)(phenoxy)phosphoryl)amino)propanoate (674 mg, 0.955 mmol) in anhydrous THF (15 mL) was added dropwise and stirred at −15° C. for 4 h. LCMS showed the reaction was completed. The reaction mixture was quenched with 2N NH₄Cl (10 mL) and diluted with EtOAc (200 mL). The organic layer was washed with water (40 mL), brine (40 mL), dried over Na₂SO₄, filtered and concentrated. The residue was purified by reverse phase chromatography (SepaFlash® C18 column, BOSTON, 40 g, 0-100 CH₃CN/10 mM NH₄HCO₃ H₂O) to give tridecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (209 mg, 0.255 mmol, 37.4% yield) as a white solid. LCMS (M+H)=814.8; Retention time (10 mM NH₄HCO₃)=2.56 min; purity: 100% (254 nm). HPLC Retention time (10 mM NH₄HCO₃)=8.26 min. ¹H NMR (400 MHz, CDCl₃) δ 7.93 (s, 1H), 7.30-7.24 (m, 2H), 7.15-7.10 (m, 3H), 6.66-6.58 (m, 3H), 6.32 (dd, J=7.6, 4.0 Hz, 1H), 6.19 (brs, 2H), 4.78 (dd, J=12.8, 7.2 Hz, 1H), 4.33 (ddd, J=26.0, 11.6, 7.2 Hz, 2H), 4.25-4.16 (m, 2H), 4.02 (t, J=6.8 Hz, 2H), 3.76 (d, J=5.6 Hz, 1H), 2.96 (d, J=5.6 Hz, 2H), 2.80 (ddd, J=13.6, 7.2, 4.4 Hz, 1H), 2.73 (s, 1H), 2.72-2.66 (m, 1H), 1.51 (quint, J=6.8 Hz, 2H), 1.28-1.20 (m, 20H), 0.88 (t, J=6.8 Hz, 3H).

### Example 56

### Heptadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

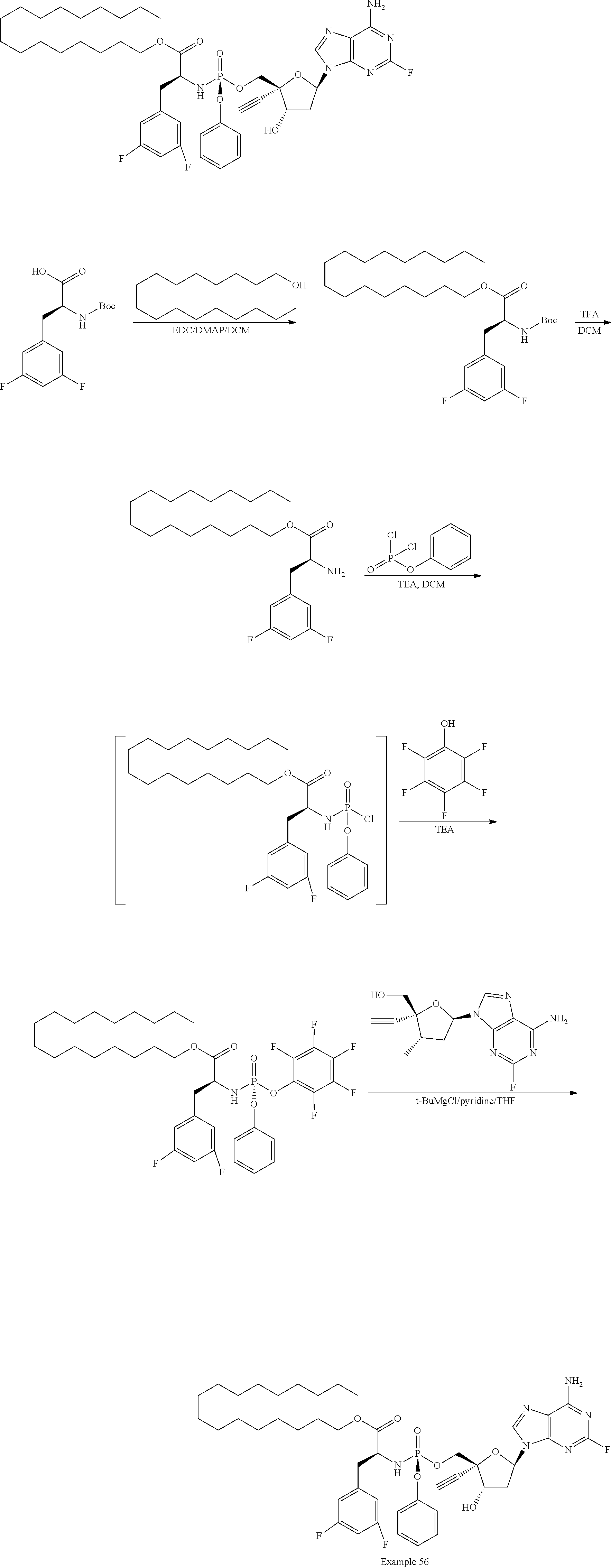

### Step 1: Heptadecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (4 g, 13.28 mmol) in DCM (80 mL) was added DMAP (0.162 g, 1.328 mmol) and EDC (3.31 g, 17.26 mmol) at 0° C. After 30 min, heptadecan-1-ol (4.43 g, 17.26 mmol) was added and the resulting reaction mixture was stirred at 25° C. for 16 h. LCMS showed the reaction was completed. The reaction mixture was diluted with water (50 mL) and extracted with DCM (2×100 mL). The combined organic layers were washed with brine (50 mL), dried over Na₂SO₄, filtered and concentrated to afford heptadecyl (S)-2-((tert-butoxycarbonyl)amino)-3-(3,5-difluorophenyl)propanoate (6.3 g, 11.67 mmol, 88% yield) as a yellow solid which was used in the next step without further purification. LCMS: (M+Na)=563.2; Retention time (0.1% TFA)=4.62 min.

### Step 2: Heptadecyl (S)-2-amino-3-(3,5-difluorophenyl)propanoate

To a stirred solution of heptadecyl (S)-2-((tert-butoxycarbonyl)amino)-3-(3,5-difluorophenyl)propanoate (5.8 g, 10.75 mmol) in DCM (100 mL) was added TFA (20 mL, 260 mmol) dropwise at 0° C. The resulting reaction mixture was stirred at 25° C. for 4 h. LCMS showed the reaction was completed. The reaction was concentrated and the residue was neutralized with NaHCO₃ (40 mL) and extracted with DCM (3×100 mL). The combined organic layers were washed with brine (50 mL), dried over Na₂SO₄, filtered and concentrated. The residue was purified by silica gel chromatography (petroleum ether/ethyl acetate=1/1) to give heptadecyl (S)-2-amino-3-(3,5-difluorophenyl)propanoate (4.4 g, 10.01 mmol, 93% yield) as a light yellow solid. LCMS (M+H)=440.2; Retention time (0.1% TFA)=2.31 min.

### Step 3: heptadecyl (S)-3-(3,5-difluorophenyl)-2-(((S)-(perfluorophenoxy)(phenoxy)phosphoryl)amino)propanoate

To a stirred solution of heptadecyl (S)-2-amino-3-(3,5-difluorophenyl)propanoate (4.4 g, 10.01 mmol) in DCM (100 mL) was added triethylamine (1.530 mL, 11.01 mmol) dropwise at −70° C. To this mixture was added a solution of phenyl phosphorodichloridate (2.090 g, 9.91 mmol) in anhydrous DCM (10 mL) dropwise. The reaction mixture was stirred at this temperature for additional 30 min and then allowed to warm to 0° C. over 2 h and stirred for 1 h. To this mixture was added a solution of 2,3,4,5,6-pentafluorophenol (1.842 g, 10.01 mmol) and triethylamine (1.530 mL, 11.01 mmol) in DCM (20 mL) dropwise. The crude mixture was allowed to stir at 0° C. for 16 h. LCMS showed the reaction was completed. The white solid (triethylamine hydrochloride) was filtered off and washed with DCM (1×30 mL). The filtrate was concentrated under reduced pressure, the residue was triturated with tert-butyl mether ether (200 mL) and the triethylamine hydrochloride salt was removed by filtration. The cake was washed with tert-butyl mether ether (2×20 mL) and the filtrate was concentrated under reduced pressure. The residue was triturated with 15% EtOAc in hexanes (80 mL) and solids collected by filtration heptadecyl (S)-3-(3,5-difluorophenyl)-2-(((S)-(perfluorophenoxy)(phenoxy)phosphoryl)amino)propanoate (1.6 g, 2.100 mmol, 20.99% yield) as a white solid (>98% de as determined by ³¹P NMR). LCMS (M+H)=762.1; Retention time (0.1% TFA)=4.70 min. ³¹PNMR (CDCl₃, 162 MHz) δ −1.60.

### Step 4: Heptadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40.00 mL) and pyridine (10 mL) was added tert-butylmagnesium chloride (159 mg, 1.364 mmol) dropwise at −15° C. After 1 h, a solution of heptadecyl (S)-3-(3,5-difluorophenyl)-2-(((S)-(perfluorophenoxy)(phenoxy)phosphoryl)amino)propanoate (727 mg, 0.955 mmol) in anhydrous THF (20 mL) was added dropwise and stirred at −15° C. for 4 h. LCMS showed the reaction was completed. The reaction mixture was quenched with 2N NH₄Cl (10 mL) and the mixture was partitioned between EtOAc (150 mL) and H₂O (30 mL). The organic layer was washed with brine (30 mL), dried over Na₂SO₄, filtered and concentrated. The residue was purified by reverse phase chromatography (SepaFlash® C18 column, BOSTON, 40 g, 0-100% CH₃CN/10 mM NH₄HCO₃ H₂O) to give heptadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (172 mg, 0.193 mmol, 28.3% yield) as a pale solid. LCMS (M+H)=871.3; Retention time (0.05% TFA)=3.24 min; purity: 96.84% (254 nm). HPLC Retention time (10 mM NH₄HCO₃)=8.81 min. ¹H NMR (400 MHz, CDCl₃) δ 7.95 (s, 1H), 7.29-7.23 (m, 2H), 7.12 (t, J=8.0 Hz, 3H), 6.70-6.38 (m, 5H), 6.32 (dd, J=7.2, 4.0 Hz, 1H), 4.79 (dd, J=12.8, 7.2 Hz, 1H), 4.45 (t, J=10.8 Hz, 1H), 4.39-4.28 (m, 2H), 4.24-4.16 (m, 1H), 4.12 (d, J=5.6 Hz, 1H), 4.04-3.96 (m, 2H), 2.94 (d, J=5.6 Hz, 2H), 2.82-2.66 (m, 3H), 1.50 (quint, J=6.0 Hz, 2H), 1.28-1.18 (m, 28H), 0.88 (t, J=6.8 Hz, 3H).

### Example 57

### Dodecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

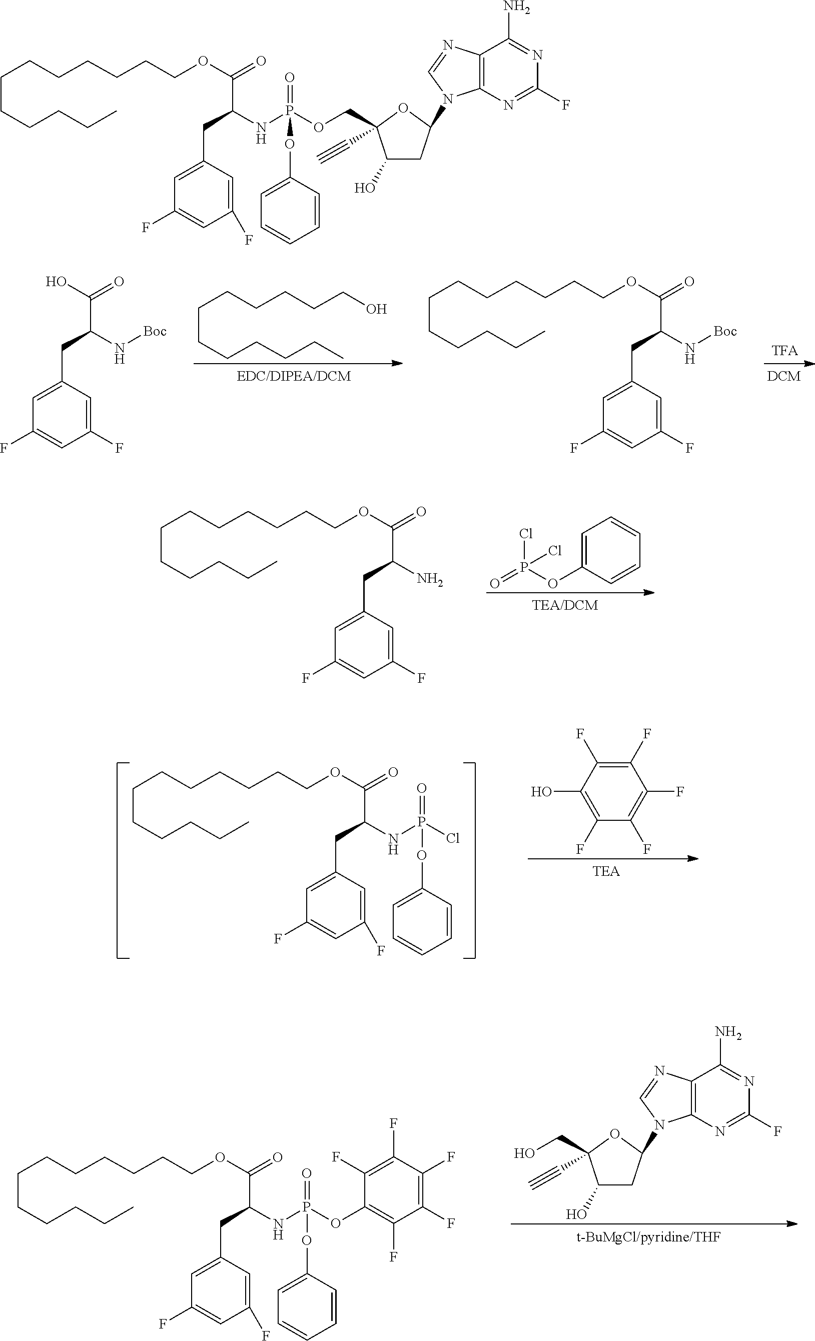

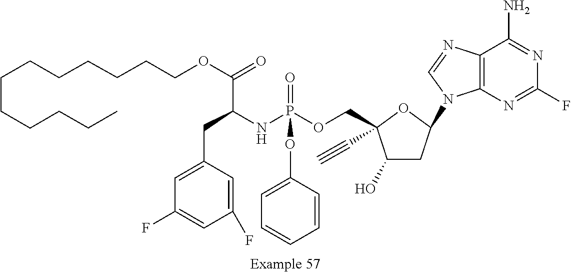 [SMILES: C#C[C@]1(CO[P@@](=O)(N[C@@H](Cc2cc(F)cc(F)c2)C(=O)OCCCCCCCCCCCC)Oc2ccccc2)O[C@@H](n2cnc3c(N)nc(F)nc32)C[C@@H]1O]

### Step 1: Dodecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (5 g, 16.60 mmol), dodecan-1-ol (3.09 g, 16.60 mmol) and EDC (4.14 g, 21.57 mmol) in DCM (50 mL) was added N,N-dimethylpyridin-4-amine (2.027 g, 16.60 mmol) at 0° C. The resulting reaction mixture was stirred at 25° C. for 16 h. LCMS showed the reaction was completed. The mixture was diluted with water (80 mL) and extracted with DCM (100 mL) twice. The combined organic phases were washed with brine (100 mL) twice, dried over Na₂SO₄ and concentrated to give dodecyl (S)-2-((tert-butoxycarbonyl)amino)-3-(3,5-difluorophenyl)propanoate (5.6 g, 11.92 mmol, 71.9% yield) as a white solid which was used in the next step without further purification. LCMS (M+Na)=492.1; Retention time (0.1% TFA)=2.954.

### Step 2: Dodecyl (S)-2-amino-3-(3,5-difluorophenyl)propanoate

To a stirred solution of dodecyl (S)-2-((tert-butoxycarbonyl)amino)-3-(3,5-difluorophenyl)propanoate (5.6 g, 11.92 mmol) in DCM (50 mL) was added TFA (10 mL) at 0° C. The mixture was stirred at room temperature for 16 h. LCMS showed the reaction completed. The mixture was concentrated under vaccum to remove most of DCM and TFA. Ice water (20 mL) was added and the pH was adjusted to 7 by aq. NaHCO₃ and extracted with EtOAc (50 mL×2). The combined organic phases were washed with brine, dried over Na₂SO₄ and concentrated. The crude product was purified by flash chromatography (silica gel, 80 g, pet. ether:EtOAc=3:1) to give dodecyl (S)-2-amino (3,5-difluorophenyl)propanoate (4 g, 7.01 mmol, 58.8% yield) as a white solid. LCMS (M+H)=370.1; Retention time (0.1% TFA)=1.965.

### Step 3: Dodecyl (S)-3-(3,5-difluorophenyl)-2-((S)-(perfluorophenoxy)(phenoxy)phosphor-yl)amino)propanoate

To a stirred solution of dodecyl (S)-2-amino-3-(3,5-difluorophenyl)propanoate (4 g, 10.83 mmol) in DCM (50 mL) was added triethylamine (1.580 mL, 11.37 mmol) at −70° C. over 15 min dropwise. To this mixture was added a solution of phenyl phosphorodichloridate (2.261 g, 10.72 mmol) in anhydrous DCM (35 mL) over 30 min. The reaction mixture was stirred at this temperature for additional 30 min and then allowed to warm to 0° C. over 2 h and stirred for 1 h. To this mixture was added a solution of 2,3,4,5,6-pentafluorophenol (1.973 g, 10.72 mmol) and triethylamine (1.655 mL, 11.91 mmol) in DCM (30 mL) over 20 min. LCMS indicated completion of reaction. The crude mixture was allowed to stir at 0° C. for 4 h and the white solid (triethylamine hydrochloride) was filtered off and washed with DCM (250 mL). The filtrate was concentrated under reduced pressure. The residue was triturated with tert-butyl mether ether (150 mL) and the triethylamine hydrochloride salt was removed by filtration. The cake was washed with tert-butyl mether ether (100 mL) and the filtrate was concentrated. The solid was triturated in pet. ether:EtOAc=5:1 (20:4 mL), filtered and washed with pet. ether:EtOAc=5:1(40:8 mL) to give dodecyl (S)-3-(3,5-difluorophenyl)-2-(((S)-(perfluorophenoxy)(phenoxy)phosphoryl)amino)propanoate (700 mg, 1.012 mmol, 9.35% yield) as white solid (>98% de as determined by ³¹PNMR). LCMS (M+H)=691.0; Retention time (10 mM NH₄CO₃)=3.010 min. ¹H NMR (400 MHz, DMSO) δ 7.31 (t, J=7.9 Hz, 2H), 7.18 (t, J=7.3 Hz, 1H), 7.03-6.99 (m, 3H), 6.94 (t, J=6.4 Hz, 2H), 4.15 (qd, J=10.3, 5.5 Hz, 1H), 4.00 (t, J=6.4 Hz, 2H), 3.09-3.00 (m, 1H), 2.85 (dd, J=13.7, 9.7 Hz, 1H), 1.47 (d, J=7.0 Hz, 2H), 1.21 (t, J=12.7 Hz, 18H), 0.83 (t, J=6.8 Hz, 3H). ³¹PNMR (DMSO-d6, 162 MHz) δ 0.21.

### Step 4: Dodecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl (hydroxymethyl)tetrahydrofuran-3-ol (200 mg, 0.682 mmol) in THF (20 mL) and pyridine (1.00 mL) was added tert-butylmagnesium chloride (1.432 mL, 1.432 mmol) dropwise at −15° C. After 2 h, a solution of dodecyl (S)-3-(3,5-difluorophenyl)-2-(((S)-(perfluorophenoxy)(phenoxy)phosphoryl)amino)propanoate (660 mg, 0.955 mmol) in THF (12.5 mL) was added dropwise and stirred at −15° C. for 4 h. LCMS showed the reaction was completed. The reaction was quenched with sat. NH₄Cl (10 mL) and filtered. The filtrate was concentrated and dissolved in EtOAc (50 mL), washed with water (30 mL) twice and the combined queous layers were extracted with EtOAc (30 mL) twice. The combined organic phases were washed with brine (30 mL), dried with Na₂SO₄, filtered and concentrated in vacuum. The crude product was purified by reverse phase chromagraphy (SepaFlash® C18 column, BOSTON, 40 g, 0-100% CH₃CN/10 mM NH₄HCO₃ H₂O) to give dodecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (131 mg, 0.164 mmol, 23.99% yield) as a white solid. LCMS (M+H)=801.1; Retention time (10 mM NH₄HCO₃)=2.584 min. ¹H NMR (400 MHz, DMSO) δ 8.23 (s, 1H), 7.83 (s, 2H), 7.25 (t, J=7.9 Hz, 2H), 7.11 (t, J=7.3 Hz, 1H), 7.01 (d, J=8.5 Hz, 2H), 6.99-6.88 (m, 3H), 6.24 (dd, J=7.6, 4.8 Hz, 1H), 6.16 (dd, J=12.8, 10.6 Hz, 1H), 5.76 (d, J=5.6 Hz, 1H), 4.53 (dd, J=12.7, 7.1 Hz, 1H), 4.07 (dd, J=10.7, 6.1 Hz, 1H), 4.02-3.95 (m, 1H), 3.84 (dddd, J=32.0, 17.2, 10.8, 6.4 Hz, 3H), 3.62 (s, 1H), 2.96 (dd, J=13.7, 3.3 Hz, 1H), 2.79 (dd, J=13.5, 9.2 Hz, 1H), 2.74-2.65 (m, 1H), 2.44 (dd, J=13.3, 7.5 Hz, 1H), 1.41-1.32 (m, 2H), 1.22 (s, 12H), 1.15 (s, 6H), 0.85 (t, J=6.8 Hz, 3H).

### Example 58

### Nonadecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

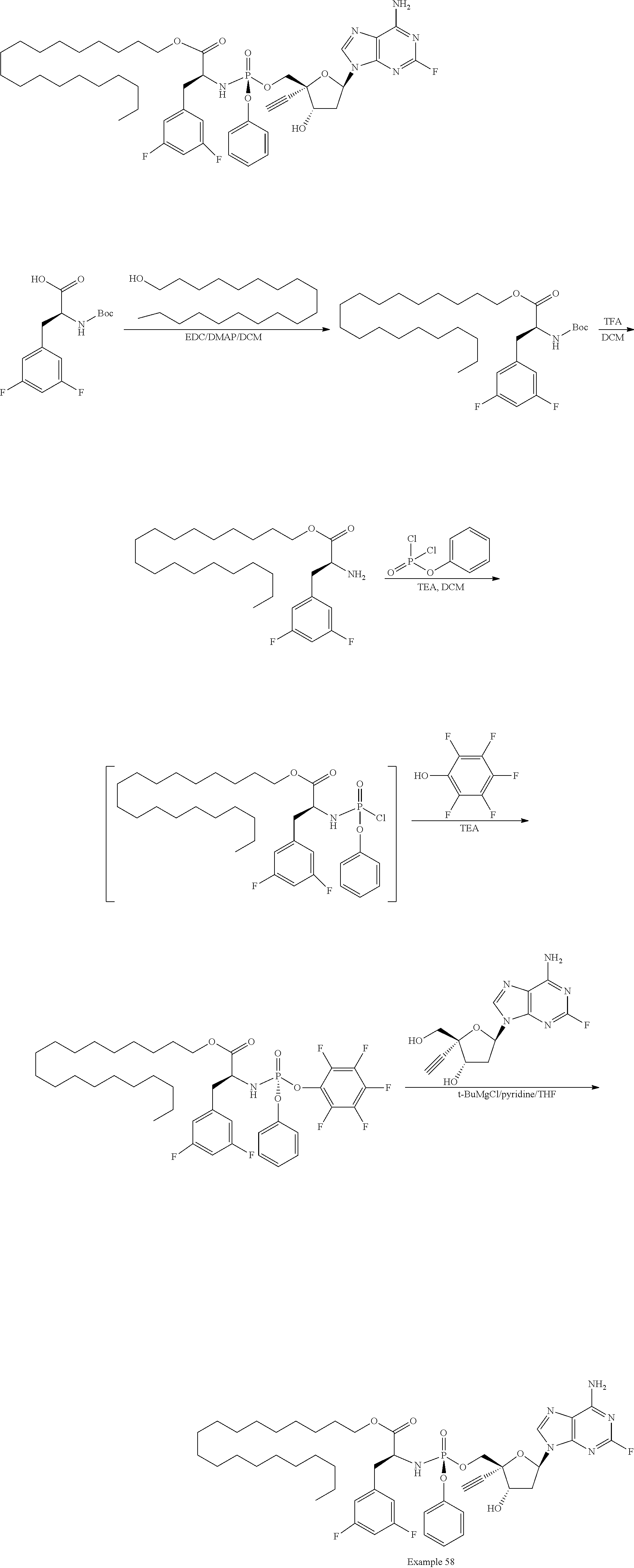

### Step 1: Nonadecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (5 g, 16.60 mmol), nonadecan-1-ol (4.72 g, 16.60 mmol) in DCM (50 mL) was added EDC (4.14 g, 21.57 mmol) and N,N-dimethylpyridin-4-amine (2.027 g, 16.60 mmol) at 0° C. The resulting reaction mixture was stirred at 25° C. for 16 h. TLC showed the reaction was completed. The mixture was concentrated under vaccum, the residue was taken up in EtOAc (50 mL) and washed with water (30 mL). The aqueous layer was back extracted with EtOAc (30 mL×2) and the combined organic phases were washed with brine (80 mL) twice, dried over Na₂SO₄ and concentrated to give nonadecyl (S)-2-((tert-butoxycarbonyl)amino)-3-(3,5-difluorophenyl)propanoate (5 g, 8.81 mmol, 53.1% yield) as a white solid which was used in the next step without further purification. LCMS (M+Na)=none; Retention time (0.1% TFA)=none.

### Step 2: Nonadecyl (S)-2-amino-3-(3,5-difluorophenyl)propanoate

To a stirred solution of nonadecyl (S)-2-((tert-butoxycarbonyl)amino)-3-(3,5-difluorophenyl)propanoate (5 g, 8.81 mmol) in DCM (50 mL) was added TFA (10 mL) at 0° C. The mixture was stirred at 25° C. for 16 h. LCMS showed the reaction was completed. The mixture was concentrated under vaccum to remove most of DCM and TFA. The residue was diluted with ice water (20 mL) and the pH was adjusted to 7 with aq. NaHCO₃ and extracted with EtOAc (50 mL×2). The combined organic phases were washed with brine (50 mL), dried over Na₂SO₄ and concentrated. The crude product was purified by flash chromatography (silica gel, 80 g, pet. ether:EtOAc=3:1) to give nonadecyl (S)-2-amino-3-(3,5-difluorophenyl)propanoate (3.6 g, 7.70 mmol, 87% yield) as a white solid. LCMS (M+H)=468.1; Retention time (0.1% TFA)=2.387.

### Step 3: Nonadecyl (S)-3-(3,5-difluorophenyl)-2-(((S)-(perfluorophenoxy)(phenoxy)phosphoryl)amino)propanoate

To a stirred solution of nonadecyl (S)-2-amino-3-(3,5-difluorophenyl)propanoate (3 g, 6.41 mmol) in DCM (50 mL) was added triethylamine (0.936 mL, 6.74 mmol) at −70° C. over 15 min dropwise. To this mixture was added a solution of phenyl phosphorodichloridate (1.340 g, 6.35 mmol) in anhydrous DCM (35 mL) over 30 min. The reaction mixture was stirred at this temperature for additional 30 min and then allowed to warm to 0° C. over 2 h and stirred for 1 h. To this mixture was added a solution of 2,3,4,5,6-pentafluorophenol (1.169 g, 6.35 mmol) and triethylamine (0.981 mL, 7.06 mmol) in DCM (30 mL) over 20 min and stirred at 0° C. for 4 h. LCMS indicated completion of reaction. The white solid (triethylamine hydrochloride) was filtered off and washed with DCM (250 mL). The filtrate was concentrated under reduced pressure. The residue was triturated with tert-butyl mether ether (150 mL) and the triethylamine hydrochloride salt was removed by filtration. The cake was washed with tert-butyl mether ether (100 mL). The solid was triturated in pet. ether:EtOAc=5:1 (25:5 mL), filtered and washed with pet. ether:EtOAc=5:1 (40:8 mL) to give nonadecyl (S)-3-(3,5-difluorophenyl)-2-(((S)-(perfluorophenoxy)(phenoxy)phosphoryl)amino)propanoate (1.1 g, 1.393 mmol, 21.71 yield) as a white solid (>98% de as determined by ³¹PNMR). ¹H NMR (400 MHz, DMSO) δ 7.24 (d, J=53.4 Hz, 3H), 6.97 (d, J=27.4 Hz, 5H), 3.99 (s, 2H), 3.07 (s, 2H), 2.86 (s, 1H), 1.48 (s, 2H), 1.21 (s, 32H), 0.84 (s, 3H). ³¹PNMR (DMSO-d6, 162 MHz) δ 0.16.

### Step 4: Nonadecyl (S)-2-((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (250 mg, 0.852 mmol) in THF (20 mL) and pyridine (1 mL) was added tert-butylmagnesium chloride (1.790 mL, 1.790 mmol) dropwise at −15° C. After 2 h, a solution of nonadecyl (S)-3-(3,5-difluorophenyl)-2-(((S)-(perfluorophenoxy)(phenoxy)phosphoryl)amino)propanoate (943 mg, 1.193 mmol) in THF (10 mL) was added dropwise to the above solution and stirred at −15° C. for 4 h. LCMS showed the reaction was completed. The reaction was quenched sat. NH₄Cl (10 mL) and filtered. The filtrate was concentrated and dissolved in EtOAc (50 mL). The organic layer was washed with water (30 mL) twice and the combined aqueous layers were back extracted with EtOAc (30 mL) twice. The combined organic phases were washed with brine (30 mL), dried with Na₂SO₄, filtered and concentrated in vacuum. The crude product was purified by reverse phase chromatography (SepaFlash® C18 column, BOSTON, 40 g, 0-100% CH3CN/10 mM NH₄HCO₃ H2O) to give nonadecyl (S)-2-(((S)-(((2R,3S,5R) (6-amino-2-fluoro-9H-purin-9-yl)-2-ethynyl-3-hydroxytetrahydrofuran yl)methoxy)(phenoxy)phosphoryl)amino)-3-(3,5-difluorophenyl)propanoate (242.5 mg, 0.270 mmol, 31.6% yield) as a white solid. LCMS (M+H)=899.0; Retention time (10 mM NH₄HCO₃)=4.061 min. ¹H NMR (400 MHz, DMSO) δ 8.23 (s, 1H), 7.84 (s, 2H), 7.25 (t, J=7.9 Hz, 2H), 7.11 (t, J=7.3 Hz, 1H), 7.03-6.99 (m, 2H), 6.99-6.88 (m, 3H), 6.23 (dd, J=7.6, 4.8 Hz, 1H), 6.16 (dd, J=12.8, 10.5 Hz, 1H), 5.76 (d, J=5.6 Hz, 1H), 4.52 (dd, J=12.8, 7.2 Hz, 1H), 4.09-3.74 (m, 5H), 3.62 (s, 1H), 2.99-2.92 (m, 1H), 2.79 (dd, J=13.5, 9.3 Hz, 1H), 2.73-2.66 (m, 1H), 2.43 (dd, J=13.4, 7.6 Hz, 1H), 1.37 (s, 2H), 1.23 (s, 26H), 1.15 (s, 6H), 0.85 (t, J=6.8 Hz, 3H).

### Example 59

### Tetradecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

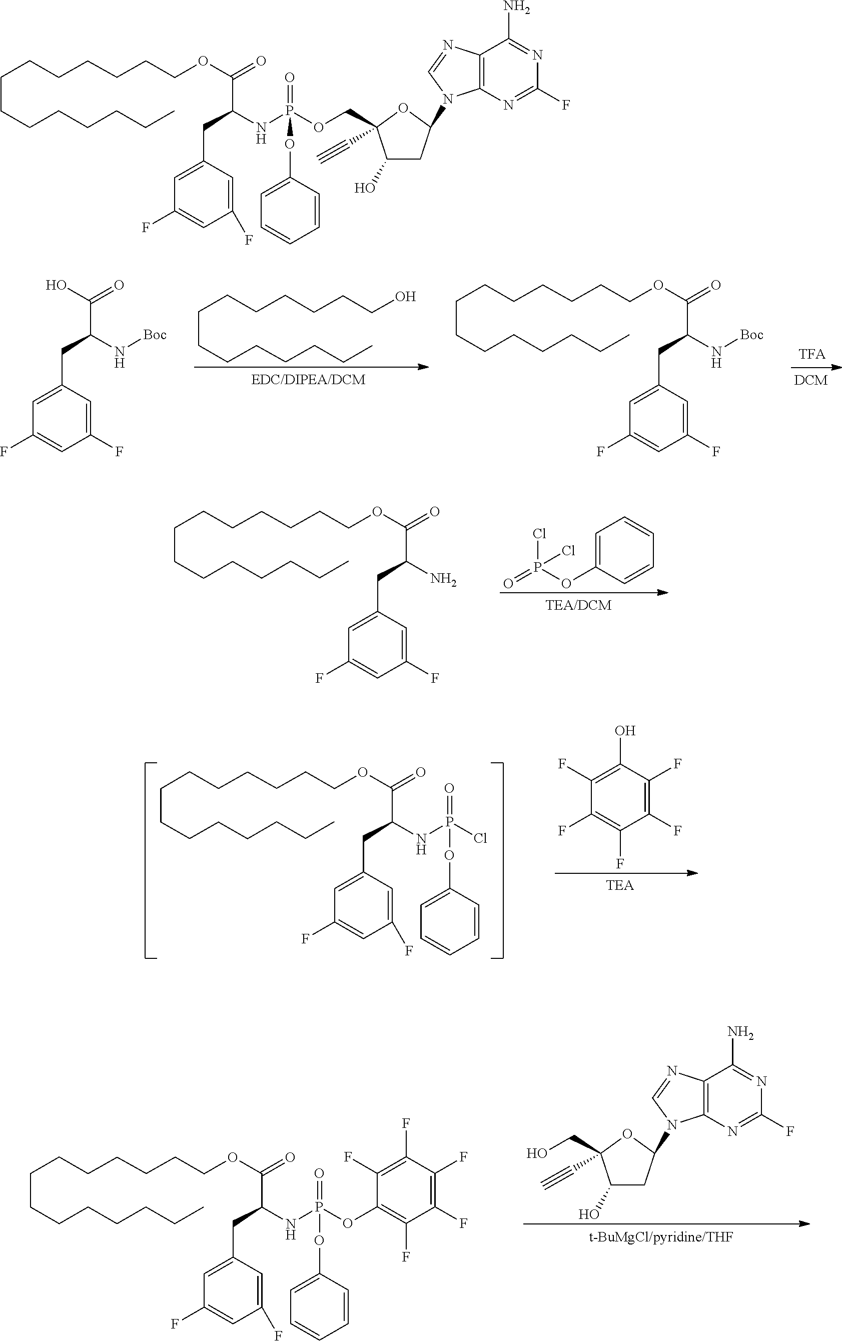

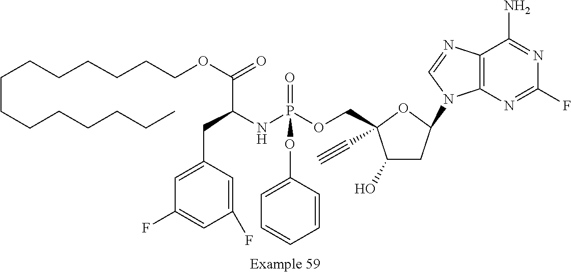 [SMILES: C#C[C@]1(CO[P@@](=O)(N[C@@H](Cc2cc(F)cc(F)c2)C(=O)OCCCCCCCCCCCCCC)Oc2ccccc2)O[C@@H](n2cnc3c(N)nc(F)nc32)C[C@@H]1O]

### Step 1: Tetradecyl (S)-2-((tert-butoxycarbonyl)amino)-3-(3,5-difluorophenyl)propanoate

To a stirred mixture of (S)-2-((tert-butoxycarbonyl)amino)-3-(3,5-difluorophenyl)propanoic acid (5 g, 16.60 mmol) in DCM (10 mL) was added DMAP (0.203 g, 1.660 mmol), tetradecan-1-ol (4.63 g, 21.60 mmol) and EDC (4.14 g, 21.61 mmol) in DCM (40 mL) was added at 0° C. The mixture was stirred at room temperature for 2 h. LCMS showed presence of new product. The reaction mixture was diluted with water (30 mL) and extracted with DCM (15 mL×3). The combined organic phases were washed with brine, dried over Na₂SO₄ and concentrated. The crude purified by silica gel chromatography (120 g, pet. ether:EtOAc=30:1) to afford tetradecyl (S)-2-((tert-butoxycarbonyl)amino)-3-(3,5-difluorophenyl)propanoate (7.2 g, 14.02 mmol, 85% yield) as white solid. LCMS (M+Na)=386.1; Retention time (10 mM NH₄HCO₃)=2.319 min.

### Step 2: Tetradecyl (S)-2-amino-3-(3,5-difluorophenyl)propanoate

To a stirred solution of tetradecyl (S)-2-((tert-butoxycarbonyl)amino)-3-(3,5-difluorophenyl)propanoate (7.2 g, 14.47 mmol) in DCM (50 mL) was added TFA (11.15 mL, 145 mmol). The resulting mixture was stirred at 5° C. for 3 h. LCMS showed the reaction was completed. The pH was adjusted to ˜8 with 1M NaOH (1 mol/L) and organic phase was separated. The aqueous layer was extracted with DCM (20 mL×2). The combined organic phases were washed with water (30 mL×2), brine (30 mL×2) dried over Na₂SO₄ and concentrated. The crude product was purified by silica gel chromatography (120 g, Santai, DCM:MeOH=10:1) to give tetradecyl (S)-2-amino-3-(3,5-difluorophenyl)propanoate (3.7 g, 7.86 mmol, 54.3% yield) as a white solid. LCMS (M+H)=398.4; Retention time (0.1% TFA)=2.59 min.

### Step 3: Tetradecyl (S)-3-(3,5-difluorophenyl)-2-((S)-(perfluorophenoxy)(phenoxy)phosphoryl)amino)propanoate

To a stirred solution of tetradecyl (S)-2-amino-3-(3,5-difluorophenyl)propanoate (3.7 g, 9.31 mmol) in anhydrous DCM (35 mL) was added Et₃N (1.362 mL, 9.77 mmol) at −70° C. over 15 min dropwise. To this mixture was added a solution of phenyl phosphorodichloridate (1.944 g, 9.21 mmol) in anhydrous DCM (35 mL) over 1 h. The reaction mixture was stirred at this temperature for additional 30 min and then allowed to warm to 0° C. over 2 h and stirred for 1 h. To this mixture was added a solution of Et₃N (1.427 mL, 10.24 mmol) and 2,3,4,5,6-pentafluorophenol (1.696 g, 9.21 mmol) in DCM (30 mL) over 20 min. The reaction mixture was stirred at this temperature 45 min. LCMS showed the reaction was completed. The white solid (triethylamine hydrochloride) was filtered off and washed with DCM (1×25 mL). The filtrate was concentrated under reduced pressure, the residue was triturated with tert-butyl mether ether (25 mL) and the triethylamine hydrochloride salt was removed by filtration. The cake was washed with tert-butyl mether ether (25 mL), and the combined filtrate was concentrated under reduced pressure. The crude solid was triturated in pet. ether:EtOAc=5:1 (30:6 mL), filtered and washed with pet. ether:EtOAc=5:1 (40:8 mL) to give tetradecyl (S)-3-(3,5-difluorophenyl)-2-(((S)-(perfluorophenoxy)(phenoxy)phosphoryl)amino)propanoate (1.2 g, 1.667 mmol, 17.92% yield) as a white solid (>98% de as determined by ³¹PNMR). LCMS (M+H)=719.1; Retention time (0.1% TFA)=3.57 min. ³¹PNMR (CDCl₃, 162 MHz) δ −1.60.

### Step 4: Tetradecyl (S)-2-((S)-(((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl (hydroxymethyl)tetrahydrofuran-3-ol (250 mg, 0.852 mmol) in THF (50 mL) and pyridine (12.50 mL) was added tert-butylmagnesium chloride (1.790 mL, 1.790 mmol) dropwise at 0° C. Then the reaction was stirred at 0° C. for 30 min. A solution of tetradecyl (S)-3-(3,5-difluorophenyl)-2-(((S)-(perfluorophenoxy)(phenoxy)phosphoryl)amino)propanoate (859 mg, 1.193 mmol) in THF (12.5 mL) was added dropwise to the above solution at −15° C. Then, the reaction was stirred at −15° C. for 16 h. LCMS showed the reaction was completed. The reaction was diluted with sat. NH₄C₁ (20 mL) and extracted with EtOAc (50 mL×3). The combined organic layers were washed with brine (50 mL), dried with Na₂SO₄, filtered and concentrated in vacuum. The crude product was purified by prep-HPLC (basic) (A: water (10 mM NH₄HCO₃), B: CH3CN, (0-90% B over 8 min, stop at 16 min)) to give tetradecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (203.4 mg, 0.241 mmol, 28.2% yield) as a white solid. LCMS (M+H)=829.0; Retention time (10 mM NH₄HCO₃)=2.84 min. ¹H NMR (400 MHz, DMSO) δ 8.23 (s, 1H), 7.85 (s, 2H), 7.25 (t, J=7.9 Hz, 2H), 7.10 (t, J=7.3 Hz, 1H), 7.01 (d, J=8.5 Hz, 2H), 6.97-6.89 (m, 3H), 6.24 (dd, J=7.6, 4.8 Hz, 1H), 6.16 (dd, J=12.8, 10.6 Hz, 1H), 5.77 (d, J=5.6 Hz, 1H), 4.53 (dd, J=12.8, 7.1 Hz, 1H), 4.09-3.95 (m, 2H), 3.93-3.77 (m, 3H), 3.61 (s, 1H), 3.00-2.92 (m, 1H), 2.79 (dd, J=13.5, 9.2 Hz, 1H), 2.74-2.66 (m, 1H), 2.48-2.41 (m, 1H), 1.42-1.33 (m, 2H), 1.18 (d, J=25.2 Hz, 22H), 0.83 (t, J=6.8 Hz, 3H).

### Example 60

### Henicosyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

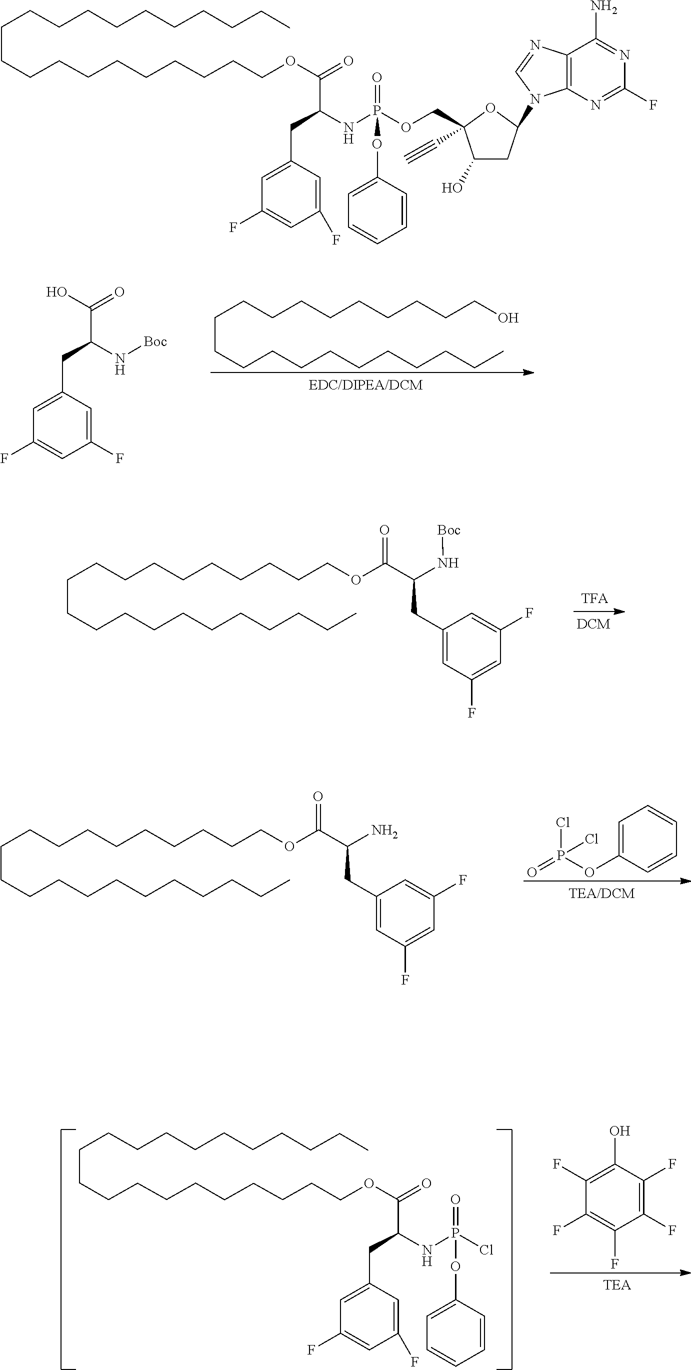

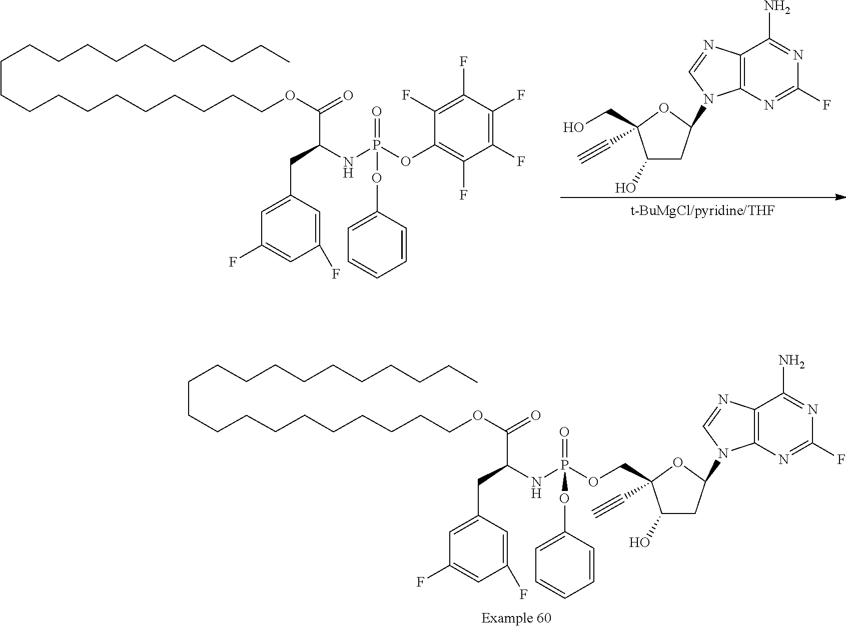 [SMILES: CCCCCCCCCCCCCCCCCCCCCOC(=O)[C@H](Cc1cc(F)cc(F)c1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2cc(F)cc(F)c2)C(=O)OCCCCCCCCCCCCCCCCCCCCC)Oc2ccccc2)O[C@@H](n2cnc3c(N)nc(F)nc32)C[C@@H]1O]

### Step 1: Henicosyl (S)-2-((tert-butoxycarbonyl)amino)-3-(3,5-difluorophenyl)propanoate

To a stirred mixture of (S)-2-((tert-butoxycarbonyl)amino)-3-(3,5-difluorophenyl)propanoic acid (3.7 g, 12.28 mmol) in DCM (20 mL) was added DMAP (0.150 g, 1.228 mmol) at 0° C., and the mixture was stirred at 0° C. for 0.5 h. Henicosan-1-ol (5.00 g, 15.98 mmol) and EDC (3.07 g, 15.99 mmol) in DCM (40 mL) was added at 0° C. The mixture was stirred at room temperature for 2 h. LCMS showed the completion of reaction. The reaction mixture was diluted with water (50 mL) and extracted with DCM (30 mL×3). The combined organic phases were washed with brine, dried over Na₂SO₄ and concentrated to give henicosyl (S)-2-((tert-butoxycarbonyl)amino)-3-(3,5-difluorophenyl)propanoate (7 g, 11.75 mmol) which was used in the next step without further purification.

### Step 2: Henicosyl (S)-2-amino-3-(3,5-difluorophenyl)propanoate

To a stirred solution of henicosyl (S)-2-((tert-butoxycarbonyl)amino)-3-(3,5-difluorophenyl)propanoate (7 g, 11.75 mmol) in DCM (50 mL) was added TFA (9.05 mL, 117 mmol). The resulting mixture was stirred at 5° C. for 2.5 h. LCMS showed the reaction was completed. The pH of the reaction mixture was adjusted to ˜8 with 1M NaOH, organic phase washed with sat NaCl (50 mL), dried (Na₂SO₄) and concentrated. The crude purified by silica gel chromatography (120 g, Santai, DCM:MeOH=50:1) to give henicosyl (S)-2-amino-3-(3,5-difluorophenyl)propanoate (4.4 g, 8.63 mmol, 73.5% yield) as an oil. LCMS (M+H)=496.5; Retention time (0.1% TFA)=3.41 min.

### Step 3: Henicosyl (S)-3-(3,5-difluorophenyl)-2-(((S)-(perfluorophenoxy)(phenoxy)phosphoryl)amino)propanoate

To a stirred solution of henicosyl (S)-2-amino-3-(3,5-difluorophenyl)propanoate (4.4 g, 8.88 mmol) in anhydrous DCM (50 mL) was added triethylamine (1.299 mL, 9.32 mmol) at −75° C. over 15 min dropwise. To this mixture was added a solution of phenyl phosphorodichloridate (1.854 g, 8.79 mmol) in anhydrous DCM (50 mL) over 1 h. The reaction mixture was stirred at this temperature for additional 30 min and then allowed to warm to 0° C. over 2 h and stirred for 1 h. To this mixture was added a solution of triethylamine (1.361 mL, 9.76 mmol) and 2,3,4,5,6-pentafluorophenol (1.617 g, 8.79 mmol) in DCM (30 mL) over 20 min. The mixture was stirred for 16 h at 0° C. and 1 h at room temperature. The white solid (triethylamine hydrochloride) was filtered off and washed with DCM (1×25 mL). The filtrate was concentrated under reduced pressure, the residue was triturated with tert-butyl mether ether (50 mL) and the triethylamine hydrochloride salt was removed by filtration. The cake was washed with tert-butyl mether ether (30 mL) and the combined filtrate was concentrated. The crude solid was triturated in pet. ether:EtOAc=3:1 (30:10 mL), filtered and washed with pet. ether:EtOAc=2:1 (20:10 mL) to give henicosyl (S)-3-(3,5-difluorophenyl)-2-(((S)-(perfluorophenoxy)(phenoxy)phosphoryl)amino)propanoate (1.01 g, 1.235 mmol, 13.91 yield) as a white solid (>98% de as determined by ³¹PNMR). 1H NMR (400 MHz, CDCl₃) δ 7.37 (t, J=7.7 Hz, 2H), 7.22 (d, J=8.1 Hz, 3H), 6.67 (t, J=8.9 Hz, 1H), 6.58 (d, J=6.1 Hz, 2H), 4.47-4.39 (m, 1H), 4.15-4.04 (m, 2H), 3.85 (t, J=10.4 Hz, 1H), 3.12-3.02 (m, 2H), 1.25 (s, 38H), 0.88 (t, J=6.6 Hz, 3H). ³¹PNMR (CDCl₃, 162 MHz) δ −1.60.

### Step 4: Henicosyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (250 mg, 0.852 mmol) in THF (50 mL) and pyridine (12.50 mL) was added tert-butylmagnesium chloride (1.790 mL, 1.790 mmol) drop-wise at 0° C. Then the reaction was stirred at 0° C. for 30 min. A solution of henicosyl (S)-3-(3,5-difluorophenyl)-2-(((S)-(perfluorophenoxy)(phenoxy)phosphoryl)amino)propanoate (976 mg, 1.193 mmol) in THF (12.5 mL) was added dropwise to the above solution at −15° C. Then, the reaction was stirred at −15° C. for 16 h. TLC showed the reaction was completed. The reaction mixture was diluted with sat. NH₄Cl (20 mL) and extracted with EtOAc (50 mL×3). The combined organic layers were washed with brine (50 mL), dried with Na₂SO₄, filtered and concentrated in vacuum. The crude was purified by silica gel chromatography (SanTai, 40 g, DCM:MeOH=20:1) to give henicosyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (259.1 mg, 0.279 mmol, 32.8% yield) as white solid. ¹H NMR (400 MHz, CDCl₃) δ 8.03 (brs, 1H), 7.29-7.27 (m, 1H), 7.24 (s, 1H), 7.15-7.09 (m, 3H), 6.65-6.62 (m, 2H), 6.61-6.56 (m, 1H), 6.35-6.25 (m, 2H), 4.78 (t, J=7.6 Hz, 1H), 4.39-4.30 (m, 2H), 4.29-4.21 (m, 2H), 4.02 (dd, J=6.8, 5.7 Hz, 2H), 2.96 (d, J=5.9 Hz, 2H), 2.83-2.76 (m, 1H), 2.74-2.68 (m, 2H), 1.24 (d, J=8.3 Hz, 40H), 0.87 (t, J=6.8 Hz, 3H).

### Example 61

2-Butylhexyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

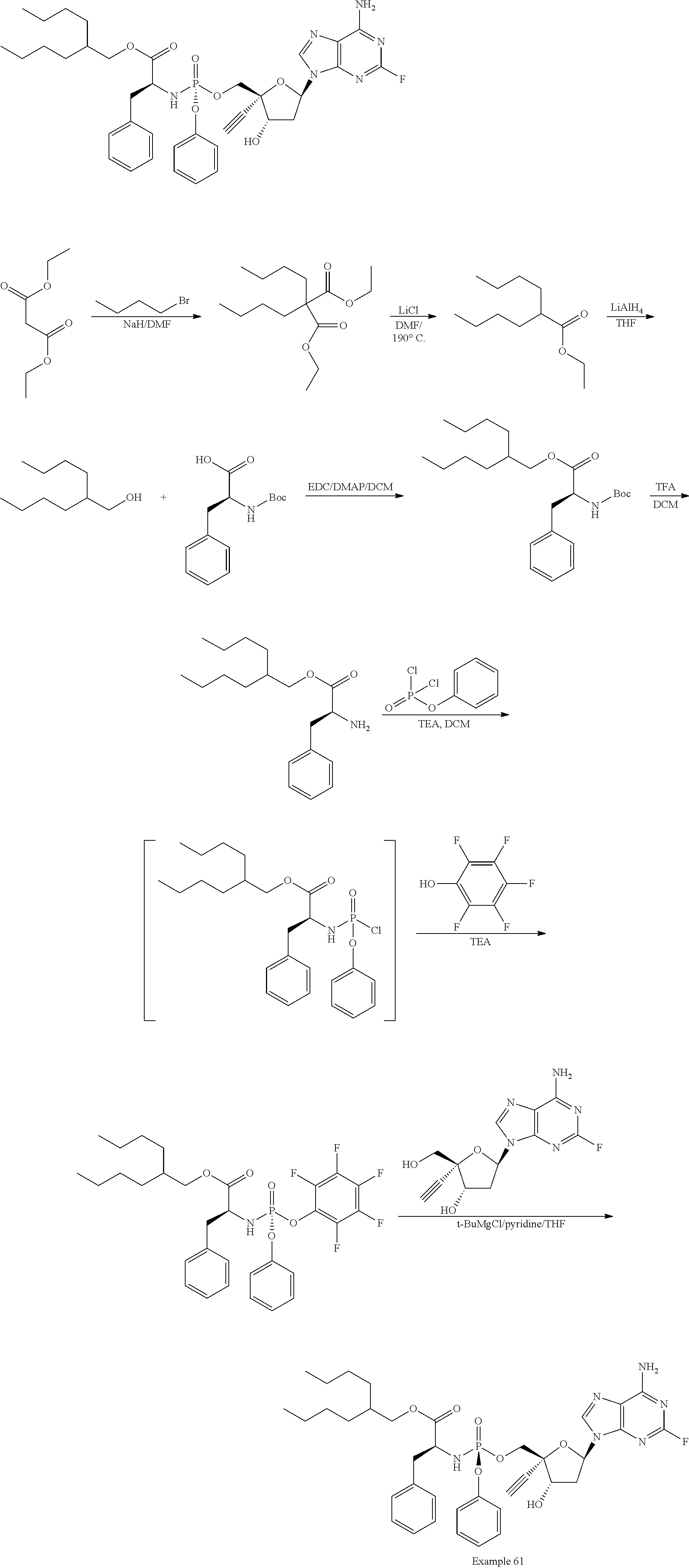

### Step 1: Diethyl 2,2-dibutylmalonate

To a stirred suspension of sodium hydride (68.7 g, 1717 mmol) in DMF (1000 mL) was added diethyl malonate (110 g, 687 mmol) dropwise at 0° C. under argon atmosphere. After 10 min, a solution of 1-bromobutane (235 g, 1717 mmol) in DMF (300 mL) was added to the mixture and the resulting mixture was stirred at 25° C. for 16 h. The reaction mixture was quenched with water (3,000 mL) and extracted with EtOAc (1,000 mL×2). The combined organic layers were washed with water (2,000 mL×3) and brine (1,000 mL), dried over Na₂SO₄, filtered and evaporated in vacuo to afford diethyl 2,2-dibutylmalonate (270 g, 734 mmol, 107% yield) which was used in the next step without further purification LCMS: (M+H)=273; Retention time (0.05% TFA)=2.05 min.

### Step 2: Ethyl 2-butylhexanoate

To a stirred mixture of diethyl 2,2-dibutylmalonate (40 g, 147 mmol) and water (2.65 mL, 147 mmol) in DMSO (150 mL) was added lithium chloride (12.45 g, 294 mmol) and stirred at 190° C. for 32 h under N₂ atmosphere. LCMS showed the reaction was completed. The mixture was cooled to room temperature and poured into ice water (600 mL) and extracted with EtOAc (200 mL×2). The combined organic layers were washed with water (300 mL×2), brine (300 mL), dried over anhydrous Na₂SO₄ and concentrated to afford ethyl 2-butylhexanoate (15 g, 48.7 mmol, 33.1% yield) as an yellow oil which was used in the next step without further purification. LCMS: (M+H)=201.3; Retention time (0.1% TFA)=2.13 min.

### Step 3: 2-Butylhexan-1-ol

A stirred mixture of ethyl 2-butylhexanoate (15 g, 74.9 mmol) and LiAlH₄ (150 mL, 150 mmol) in THF (150 mL) was heated at 70° C. for 1 h. Then, the reaction mixture was cooled to 0° C. carefully quenched with successive addition of water (5.7 mL), 15% aq. NaOH (5.7 mL) and water (17.1 mL). Anhydrous Na₂SO₄ (50 g) was added and the resulting mixture was stirred for 30 min. The resulting mixture was diluted with THF (100 mL) and the solid was removed by filtration. The filtrate was evaporated under reduced pressure to give 2-butylhexan-1-ol (10 g, 56.9 mmol, 76% yield) as an yellow oil which was used in the next step without further purification. ¹H NMR (400 MHz, CDCl₃) δ 3.54 (d, J=5.2 Hz, 2H), 1.35-1.25 (m, 12H), 0.93-0.83 (m, 6H).

### Step 4: 2-Butylhexyl (tert-butoxycarbonyl)-L-phenylalaninate

To a stirred mixture of (tert-butoxycarbonyl)-L-phenylalanine (8.38 g, 31.6 mmol) and EDC (7.87 g, 41.1 mmol) in DCM (150 mL) was added DMAP (0.386 g, 3.16 mmol) at 0° C. After 30 min, 2-butylhexan-1-ol (5.0 g, 31.6 mmol) was added. The resulting reaction mixture was stirred at 25° C. for 2 h. TLC showed the reaction was completed. The reaction mixture was diluted with water (80 mL) and extracted with DCM (100 mL×2). The combined organic phases were washed with brine, dried over Na₂SO₄, filtered and concentrated to afford 2-butylhexyl (tert-butoxycarbonyl)-L-phenylalaninate (8.2 g, 20.22 mmol, 64.0% yield) as a yellow oil which was used in the next step without purification. ¹H NMR (400 MHz, CDCl₃) δ 7.26 (dq, J=14.3, 7.1 Hz, 3H), 7.14 (d, J=7.0 Hz, 2H), 4.98 (t, J=11.8 Hz, 1H), 4.58 (dt, J=16.2, 8.1 Hz, 1H), 4.02-3.95 (m, 2H), 3.07 (qt, J=31.7, 15.8 Hz, 2H), 1.41 (s, 9H), 1.26 (dd, J=22.6, 5.5 Hz, 12H), 0.88 (t, J=6.8 Hz, 6H).

### Step 5: 2-Butylhexyl L-phenylalaninate

To a stirred solution of 2-butylhexyl (tert-butoxycarbonyl)-L-phenylalaninate (8.2 g, 20.22 mmol) in DCM (75 mL) was added TFA (20 mL, 260 mmol) at 0° C. After 5 min, the mixture was stirred at room temperature for 2 h. TLC showed starting material was consumed. The reaction was concentrated in vacuum to give the residue, which was added water (100 mL) and adjusted pH to 8-9 with sat. Na₂CO₃. The resulting mixture was extracted with DCM (200 mL×3), washed with brine, dried over Na₂SO₄, filtered and concentrated. The residue was purified by silica gel column (DCM:MeOH=100:0\20:1) to give 2-butylhexyl L-phenylalaninate (6.0 g, 19.64 mmol, 97% yield) as white solid. ¹H NMR (400 MHz, CDCl₃) δ 7.31-7.18 (m, 5H), 4.01-3.99 (m, 2H), 3.72 (dt, J=25.8, 12.9 Hz, 1H), 3.06 (dd, J=13.5, 5.4 Hz, 1H), 2.88 (dd, J=13.5, 7.8 Hz, 1H), 1.60-1.51 (br, 2H), 1.28 (d, J=17.6 Hz, 12H), 0.89 (t, J=6.7 Hz, 6H).

### Step 6: 2-Butylhexyl ((S)-(perfluorophenoxy)(phenoxy)phosphoryl)-L-phenylalaninate

To a stirred solution of 2-butylhexyl L-phenylalaninate (7.6 g, 24.88 mmol) in anhydrous DCM (50 mL) was added triethylamine (3.80 mL, 27.4 mmol at −70° C. over 15 min dropwise. To this mixture was added a solution of phenyl phosphorodichloridate (5.20 g, 24.63 mmol) in anhydrous DCM (30 mL) over 1 h. The reaction mixture was stirred at −70° C. for additional 30 min and allowed to warm to 0° C. over 2 h and stirred at 0° C. for 1 h. To this mixture was added a solution of 2,3,4,5,6-pentafluorophenol (4.53 g, 24.63 mmol) and triethylamine (3.80 mL, 27.4 mmol in DCM (20 mL) over 20 min and stirred at 0° C. for 4 h. TLC showed the reaction was completed. The white solid (triethylamine hydrochloride) was filtered off and washed with DCM (1×50 mL). The filtrate was concentrated under reduced pressure and the residue was triturated with tert-butyl methyl ether (250 mL). Then, the triethylamine hydrochloride salt was removed by filtration. The cake was washed with tert-butyl methyl ether (2×50 mL) and the combined filtrates were concentrated. The crude product was triturated with 5% EtOAc in hexanes (40 mL) and solids collected by filtration to afford 2-butylhexyl ((S)-(perfluorophenoxy)(phenoxy)phosphoryl)-L-phenylalaninate (2.5 g, 3.98 mmol, 16.01 yield) as a white solid (>98% de as determined by ³¹PNMR). ¹H NMR (400 MHz, CDCl₃) δ 7.35 (t, J=7.9 Hz, 2H), 7.25-7.11 (m, 6H), 7.04 (dd, J=7.0, 2.4 Hz, 2H), 4.53-4.35 (m, 1H), 3.96 (ddd, J=36.3, 10.8, 5.7 Hz, 2H), 3.80-3.70 (m, 1H), 3.10 (ddd, J=20.0, 13.7, 5.8 Hz, 2H), 1.54 (s, 1H), 1.33-1.11 (m, 12H), 0.88 (t, J=6.5 Hz, 6H). ³¹PNMR (CDCl₃, 162 MHz) δ −1.53.

### Step 7: 2-Butylhexyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.2 g, 0.682 mmol) in THF (40 mL) and pyridine (2.000 mL) was added tert-butylmagnesium chloride (1.432 mL, 1.432 mmol) dropwise at 0° C. Then, the reaction was stirred at 25° C. for 30 min. A solution of 2-butylhexyl ((S)-(perfluorophenoxy)(phenoxy)phosphoryl)-L-phenylalaninate (0.556 g, 0.887 mmol) in THF (20 mL) was added dropwise to the above solution at −15° C. and stirred at −15° C. for 4 h. TLC showed starting material was consumed. The reaction was quenched with 2M HCl (20 mL) and extracted with EtOAc (30 mL×3). The combined organic phases were washed with a solution of NaHCO₃ (20 mL), brine (20 mL), dried with Na₂SO₄, filtered and concentrated. The residue was purified by silica gel chromatography (25 g, DCM/MeOH=100/0 to 10/1) to afford 2-butylhexyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (108 mg, 0.141 mmol, 20.63% yield) as white solid. LCMS (M+H)=737; Retention time (0.1% TFA)=1.94 min. ¹H NMR (400 MHz, CDCl₃) δ 7.94 (s, 1H), 7.26-7.05 (m, 10H), 6.36-6.26 (m, 1H), 6.26-5.91 (m, 2H), 4.72 (t, J=7.5 Hz, 1H), 4.37-4.14 (m, 3H), 3.95 (d, J=5.7 Hz, 3H), 3.00 (d, J=6.3 Hz, 2H), 2.81-2.59 (m, 3H), 1.53 (s, 1H), 1.21 (s, 12H), 0.86 (t, J=6.7 Hz, 6H).

### Example 62

### 2-Pentylheptyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

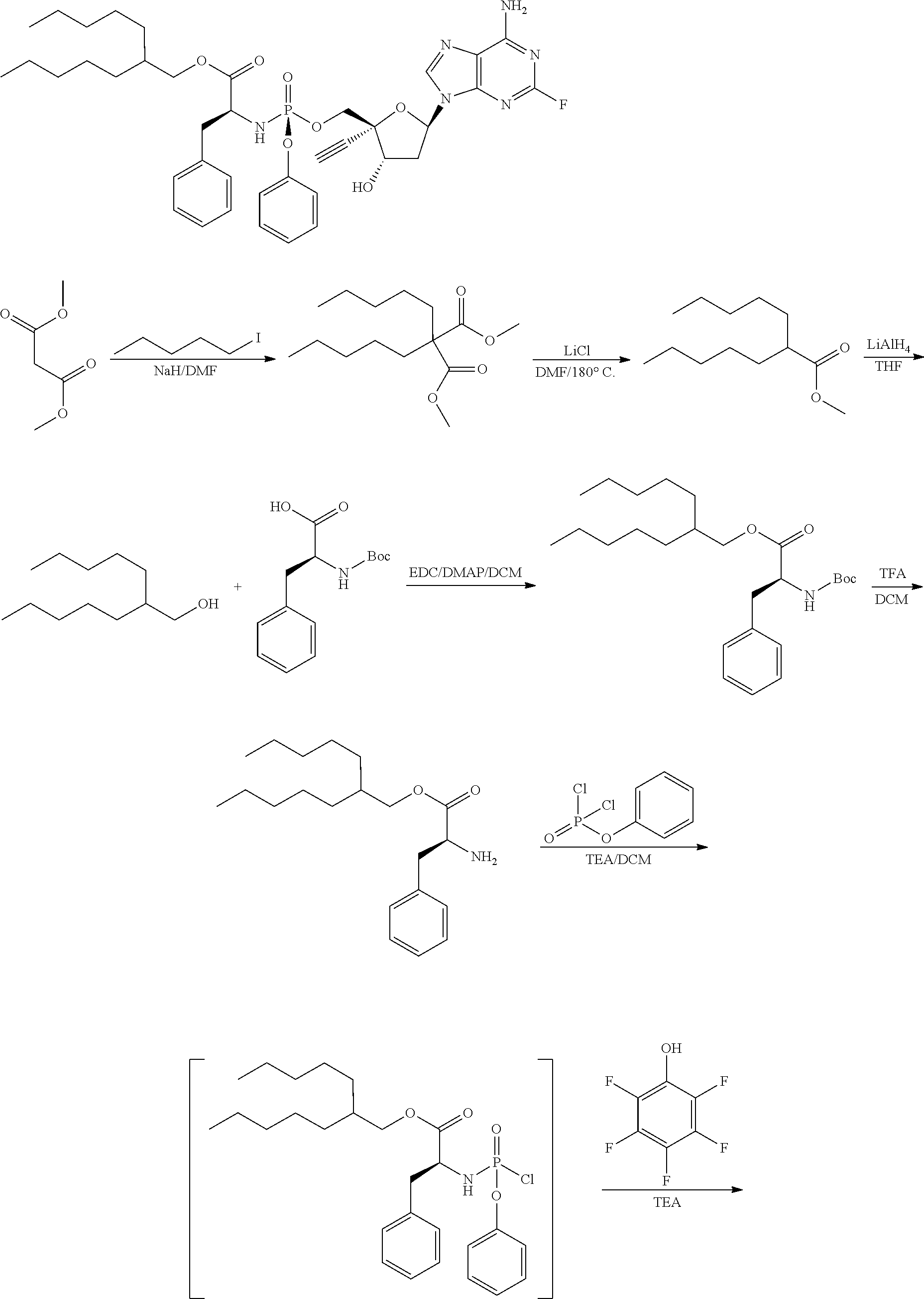

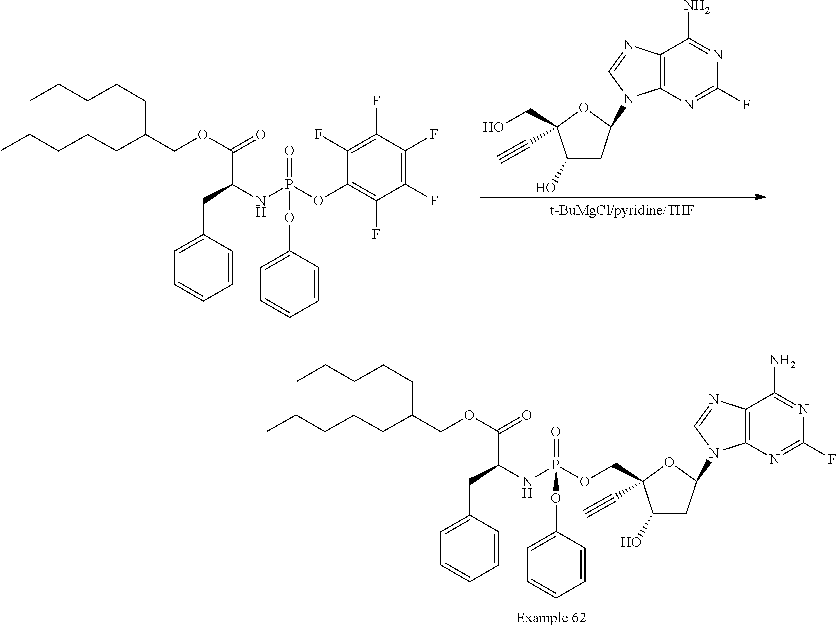 [SMILES: CCCCCC(CCCCC)COC(=O)[C@H](Cc1ccccc1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2ccccc2)C(=O)OCC(CCCCC)CCCCC)Oc2ccccc2)O[C@@H](n2cnc3c(N)nc(F)nc32)C[C@@H]1O]

### Step 1: Dimethyl 2,2-dipentylmalonate

To a stirred suspension of sodium hydride (7.57 g, 189 mmol) in DMF (100 mL) was added dimethyl malonate (10 g, 76 mmol) dropwise at 0° C. under N₂ atmosphere. After 30 min, a solution of 1-iodopentane (37.5 g, 189 mmol) in DMF (30 mL) was added and the resulting mixture was stirred at room temperature for 16 h. The reaction mixture was quenched with water (200 mL) and extracted with diethyl ether (80 mL) twice. The combined organic layers were washed with water (100 mL×2) and brine (100 mL×2), dried over Na₂SO₄, filtered and evaporated in vacuum to afford dimethyl 2,2-dipentylmalonate (27 g, 49.6 mmol, 65.5% yield) which was used in the next step without purification. LCMS (M+H)=273.2, Retention time (0.05% TFA): 2.66 min.

### Step 2: Methyl 2-pentylheptanoate

A stirred mixture of dimethyl 2,2-dipentylmalonate (27 g, 99 mmol), lithium chloride (8.40 g, 198 mmol) and water (1.786 mL, 99 mmol) in DMSO (150 mL) was heated at 180° C. under N₂ atmosphere for 6 h. LCMS showed the reaction was completed. The mixture was cooled to room temperature and diluted with H₂O (200 mL). The mixture was extracted with EtOAc (80 mL) twice and combined organic layers were washed with brine (200 mL), dried over Na₂SO₄ and concentrated to obtain methyl 2-pentylheptanoate (16 g, 39.6 mmol, 39.9% yield) which was used in the next step without purification. LCMS (M+H)=215.3; Retention time (0.05% TFA): 2.86 min.

### Step 3: 2-Pentylheptan-1-ol

To a stirred solution of methyl 2-pentylheptanoate (16 g, 74.6 mmol) in THF (150 mL) was added 1M LiAlH₄/THF (149 mL, 149 mmol) dropwise at room temperature. The mixture was stirred at 70° C. for 2 h and cooled to 0° C. The reaction mixture was quenched with successive addition of water (5 mL), 15% aq. NaOH (5 mL) and water (15 mL). The resulting mixture was diluted with EtOAc and the precipitate was removed by filtration. The filtrate was evaporated under reduced pressure to obtain 2-pentylheptan-1-ol (14 g, 67.6 mmol, 91% yield) as light yellow oil which was used in the next step without purification. ¹H NMR (400 MHz, CDCl₃) δ 3.52 (d, J=5.5 Hz, 2H), 1.32-1.22 (m, 17H), 0.88 (t, J=6.9 Hz, 6H).

### Step 4: 2-Pentylheptyl (tert-butoxycarbonyl)-L-phenylalaninate

To a stirred mixture of (tert-butoxycarbonyl)-L-phenylalanine (9.75 g, 36.8 mmol) and EDC (9.16 g, 47.8 mmol) in DCM (150 mL) was added DMAP (0.449 g, 3.68 mmol) at 0° C. After 30 min, 2-pentylheptan-1-ol (6.85 g, 36.8 mmol) was added and the resulting reaction mixture was stirred at 25° C. for 16 h. TLC showed the reaction was completed. The reaction mixture was diluted with water (80 mL) and extracted with DCM (100 mL×2). The combined organic phases were washed with brine, dried over Na₂SO₄ and concentrated. The residue was purified by silica gel chromatography (pet. ether:EtOAc=10:1) to afford 2-pentylheptyl (tert-butoxycarbonyl)-L-phenylalaninate (16 g, 36.9 mmol, 100% yield) as a yellow oil. ¹H NMR (400 MHz, CDCl₃) δ 7.30-7.22 (m, 3H), 7.12 (d, J=1.6 Hz, 2H), 4.98 (t, J=4.0 Hz, 1H), 4.58 (m, 1H), 4.00-3.96 (m, 2H), 3.07 (qt, J=31.7, 15.8 Hz, 2H), 1.41 (s, 9H), 1.26 (dd, J=22.6, 5.5 Hz, 16H), 0.88 (t, J=6.8 Hz, 6H).

### Step 5: 2-Pentylheptyl L-phenylalaninate

To a stirred solution of 2-pentylheptyl (tert-butoxycarbonyl)-L-phenylalaninate (16.0 g, 36.9 mmol) in DCM (80 mL) was added TFA (40 mL, 519 mmol) at 0° C. After 5 min, the mixture was warmed to room temperature and stirred for 2 h. TLC showed the reaction was completed. The reaction was concentrated to dryness, water (80 mL) was added and the pH was adjusted to 8-9 with sat. Na₂CO₃, extracted with DCM (80 mL×3). The combined organic phases were washed with brine (100 mL), dried over Na₂SO₄, filtered and concentrated. The residue was purified by silica gel chromatography (DCM:MeOH=100:0 to 20:1) to afford 2-pentylheptyl L-phenylalaninate (8.6 g, 25.8 mmol, 69.9% yield) as yellow oil. ¹H NMR (400 MHz, CDCl₃) δ 7.33-7.27 (m, 2H), 7.22 (ddd, J=14.6, 6.7, 4.0 Hz, 3H), 4.08-3.94 (m, 2H), 3.88-3.64 (m, 1H), 3.19-3.01 (m, 1H), 2.92-2.80 (m, 1H), 1.61 (d, J=5.4 Hz, 1H), 1.49 (s, 2H), 1.35-1.20 (m, 16H), 0.89 (t, J=6.9 Hz, 6H).

### Step 6: 2-Pentylheptyl ((S)-(perfluorophenoxy)(phenoxy)phosphoryl)-L-phenylalaninate

To a stirred solution of 2-pentylheptyl L-phenylalaninate (8.2 g, 24.59 mmol) in anhydrous DCM (50 mL) was added triethylamine (3.76 mL, 27.0 mmol) at −70° C. over 15 min dropwise. To this mixture was added a solution of phenyl phosphorodichloridate (5.14 g, 24.34 mmol) in anhydrous DCM (30 mL) over 1 h. The reaction mixture was stirred at −70° C. for additional 30 min and allowed to warm to 0° C. over 2 h and stirred for another 1 h at to 0° C. To this mixture was added a solution of 2,3,4,5,6-pentafluorophenol (4.48 g, 24.34 mmol) and triethylamine (3.76 mL, 27.0 mmol) in DCM (20 mL) over 20 min and stirred at 0° C. for 4 h. TLC showed the reaction was completed. The white solid (triethylamine hydrochloride) was filtered off and washed with DCM (50 mL). The filtrate was concentrated under reduced pressure and the residue was triturated with tert-butyl methyl ether (250 mL). The triethylamine hydrochloride salt was removed by filtration. The cake was washed with tert-butyl methyl ether (2×50 mL) and the combined filtrates were concentrated. The crude product was triturated with hexanes (50 mL) and solids collected to give 2-pentylheptyl ((S)-(perfluorophenoxy)(phenoxy)phosphoryl)-L-phenylalaninate (4.2 g, 6.21 mmol, 25.3% yield) as a white solid (>97% de as determined by ³¹PNMR). ¹H NMR (400 MHz, CDCl₃) δ 7.35 (t, J=7.9 Hz, 2H), 7.25-7.15 (m, 6H), 7.04 (dd, J=7.0, 2.4 Hz, 2H), 4.45 (dd, J=10.4, 4.8 Hz, 1H), 3.96 (ddd, J=35.1, 10.8, 5.7 Hz, 2H), 3.83-3.71 (m, 1H), 3.10 (ddd, J=20.0, 13.7, 5.7 Hz, 2H), 1.55 (s, 1H), 1.36-1.07 (m, 16H), 0.88 (dd, J=7.1, 6.3 Hz, 6H). ³¹PNMR (CDCl₃, 162 MHz) δ −1.53.

### Step 7: 2-Pentylheptyl ((S)-(((2R,3S,5R)-5-(6-amino-2-fluoro-9H-purin-9-yl)-2-ethynyl-3-hydroxytetrahydrofuran-2-yl)methoxy) (phenoxy)phosphoryl)-L-phenylalaninate

To a stirred solution of (2R,3S,5R)-5-(6-amino-2-fluoro-9H-purin-9-yl)-2-ethynyl (hydroxymethyl)tetrahydrofuran-3-ol (0.2 g, 0.682 mmol) in THF (40 mL) and pyridine (2.000 mL) was added tert-butylmagnesium chloride (1.432 mL, 1.432 mmol) dropwise at 0° C. Then, the reaction was stirred at 25° C. for 30 min. A solution of 2-pentylheptyl ((S)-(perfluorophenoxy)(phenoxy)phosphoryl)-L-phenylalaninate (0.581 g, 0.887 mmol) in THF (20 mL) was added dropwise to the above solution at −15° C. and stirred at −15° C. for 4 h. TLC showed starting material was consumed. The reaction was quenched with 2M HCl (20 mL) and extracted with EtOAc (30 mL×3). The combined organic phases were washed with a solution of NaHCO₃ (20 mL), brine (20 mL), dried with Na₂SO₄, filtered and concentrated. The crude product was purified by flash chromatography (25 g, DCM/MeOH=100/0 to 10/1) to afford 2-pentylheptyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (122 mg, 0.154 mmol, 22.57% yield) as white solid. LCMS (M+H)=765; Retention time (0.1% TFA)=2.06 min. ¹H NMR (400 MHz, CDCl₃) δ 7.94 (s, 1H), 7.26-7.01 (m, 10H), 6.36-6.23 (m, 1H), 6.20-5.82 (m, 2H), 4.71 (s, 1H), 4.38-4.12 (m, 3H), 3.96 (d, J=5.7 Hz, 2H), 3.85 (s, 1H), 3.70-3.46 (m, 1H), 3.01 (d, J=6.2 Hz, 2H), 2.81-2.57 (m, 3H), 1.55 (s, 1H), 1.36-1.07 (m, 16H), 0.86 (t, J=7.0 Hz, 6H).

### Example 63

### 2-Hexyloctyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

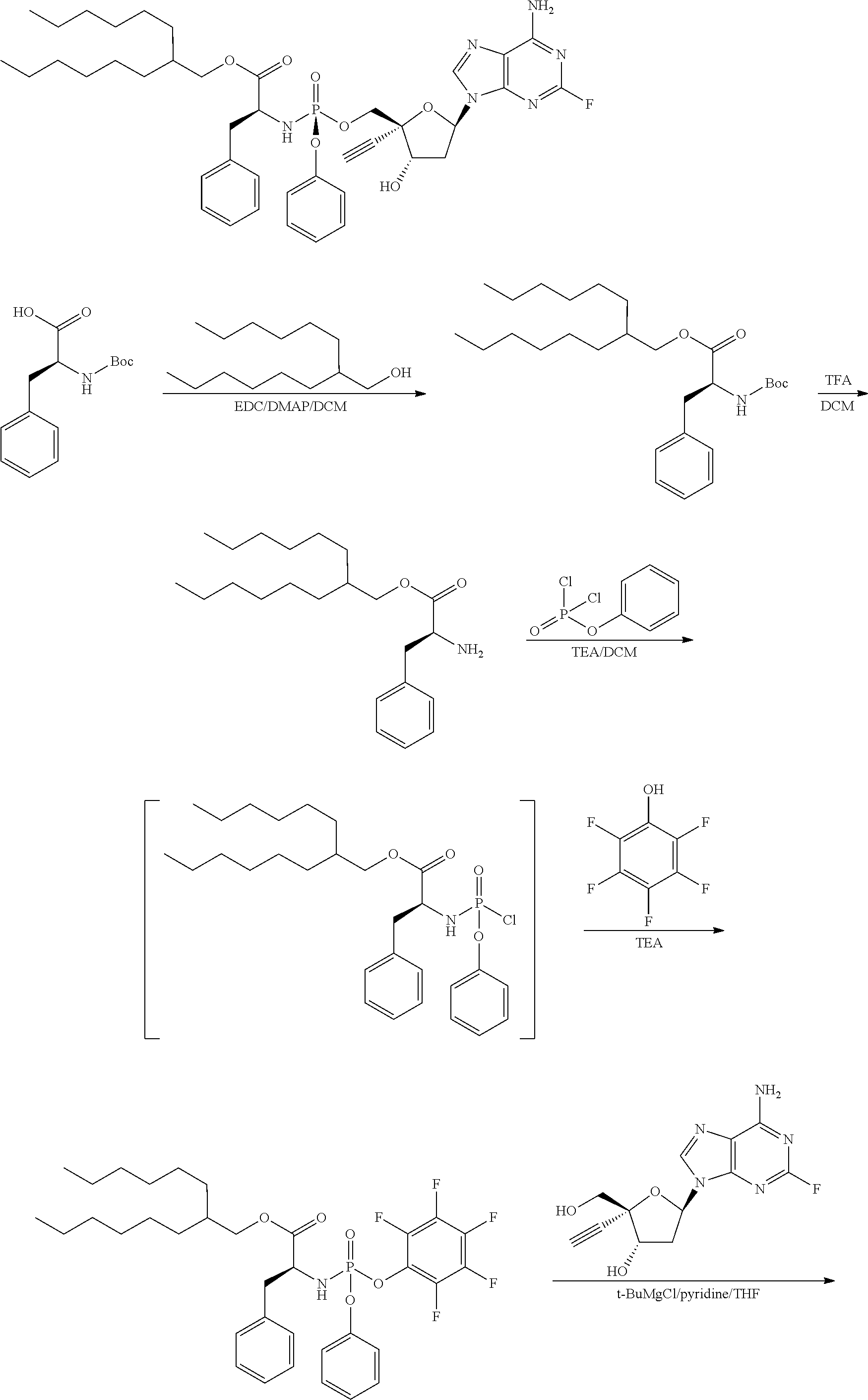

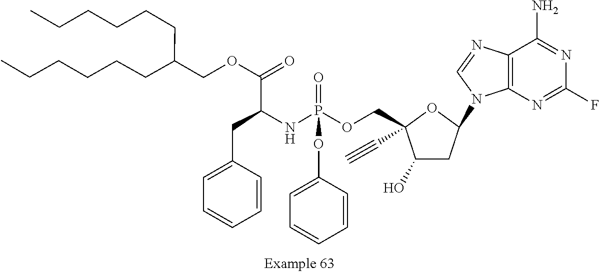 [SMILES: C#C[C@]1(CO[P@@](=O)(N[C@@H](Cc2ccccc2)C(=O)OCC(CCCCCC)CCCCCC)Oc2ccccc2)O[C@@H](n2cnc3c(N)nc(F)nc32)C[C@@H]1O]

### Step 1: 2-Hexyloctyl (tert-butoxycarbonyl)-L-phenylalaninate

To a stirred mixture of (tert-butoxycarbonyl)-L-phenylalanine (5 g, 18.85 mmol) in DCM (100 mL) was added DMAP (0.230 g, 1.885 mmol) and EDC (4.73 g, 24.69 mmol) at 0° C. After 30 min, 2-hexyloctan-1-ol (5.26 g, 24.53 mmol) was added at 0° C. and stirred at room temperature for 2 h. TLC showed starting material was consumed. The reaction mixture was diluted with water (100 mL) and extracted with DCM (150 mL×3). The combined organic phases were washed with brine, dried over Na₂SO₄ and concentrated to give 2-hexyloctyl (tert-butoxycarbonyl)-L-phenylalaninate (5.2 g, 6.76 mmol, 35.9% yield) as white solid which was used in the next step without further purification. ¹H NMR (400 MHz, CDCl₃) δ 7.26 (dq, J=14.3, 7.1 Hz, 3H), 7.13 (d, J=7.0 Hz, 2H), 4.97 (t, J=11.8 Hz, 1H), 4.58 (dt, J=16.2, 8.1 Hz, 1H), 4.26-3.90 (m, 2H), 3.07 (qt, J=31.7, 15.8 Hz, 2H), 1.42 (s, 9H), 1.28 (dd, J=22.6, 5.5 Hz, 20H), 0.88 (t, J=6.8 Hz, 6H).

### Step 2: 2-Hexyloctyl L-phenylalaninate

To a stirred solution of 2-hexyloctyl (tert-butoxycarbonyl)-L-phenylalaninate (25 g, 54.1 mmol) in DCM (75 mL was added TFA (41.7 mL, 541 mmol) at 0° C. and stirred at room temperature for 2 h. Then, the reaction was concentrated and the pH of the residue was adjusted to 8-9 with sat. Na₂CO₃ and extracted with DCM (80 mL×3). The combined organic phases were washed with brine (100 mL), dried over Na₂SO₄, filtered and concentrated. The residue was purified by silica gel chromatography (DCM:MeOH=100:0 to 20:1) to afford 2-hexyloctyl L-phenylalaninate (20 g, 52.5 mmol, 97% yield). ¹H NMR (400 MHz, CDCl₃) δ 7.39-7.01 (m, 5H), 4.05-3.94 (m, 2H), 3.72 (dt, J=25.8, 12.9 Hz, 1H), 3.08 (dd, J=13.5, 5.4 Hz, 1H), 2.87 (dd, J=13.5, 7.8 Hz, 1H), 1.57 (d, J=23.8 Hz, 2H), 1.28 (d, J=17.6 Hz, 20H), 0.89 (t, J=6.7 Hz, 6H).

### Step 3: 2-Hexyloctyl ((S)-(perfluorophenoxy)(phenoxy)phosphoryl)-L-phenylalaninate

To a stirred solution of 2-hexyloctyl L-phenylalaninate (15 g, 41.5 mmol) in anhydrous DCM (50 mL) was added triethylamine (6.34 mL, 45.6 mmol) at −70° C. over 15 min dropwise. To this mixture was added a solution of phenyl phosphorodichloridate (8.67 g, 41.1 mmol) in anhydrous DCM (30 mL) over 1 h at −70° C. The reaction mixture was stirred at this temperature for additional 30 min and then allowed to warm to 0° C. over 2 h and stirred for another 1 h. To this mixture was added a solution of 2,3,4,5,6-pentafluorophenol (7.56 g, 41.1 mmol) and triethylamine (6.34 mL, 45.6 mmol) in DCM (20 mL) over 20 min and stirred at 0° C. for 4 h. TLC showed the reaction was completed. The white solid (triethylamine hydrochloride) was filtered off and washed with DCM (50 mL). The filtrate was concentrated under reduced pressure. The residue was triturated with tert-butyl methyl ether (250 mL), and the triethylamine hydrochloride salt was removed by filtration. The cake was washed with tert-butyl methyl ether (2×50 mL) and the combined filtrates were concentrated. The crude product was triturated with hexanes (80 mL) and filtered to afford 2-hexyloctyl ((S)-(perfluorophenoxy)(phenoxy)phosphoryl)-L-phenylalaninate (2.0 g, 2.93 mmol, 7.05% yield) as a white solid (>98% de as determined by ³¹PNMR). ¹H NMR (400 MHz, CDCl₃) δ 7.35 (t, J=7.9 Hz, 2H), 7.22 (ddd, J=15.5, 6.8, 1.4 Hz, 6H), 7.04 (dd, J=7.0, 2.4 Hz, 2H), 4.50-4.36 (m, 1H), 3.96 (ddd, J=33.3, 10.8, 5.7 Hz, 2H), 3.79 (t, J=11.1 Hz, 1H), 3.10 (ddd, J=20.0, 13.7, 5.8 Hz, 2H), 1.54 (s, 1H), 1.35-1.09 (m, 20H), 0.88 (dd, J=7.0, 6.0 Hz, 6H). ³¹PNMR (CDCl₃, 162 MHz) δ −1.41.

### Step 4: 2-Hexyloctyl ((S)-(((2R,3S,5R)-5-(6-amino-24 luoro-9H-purin-9-yl)-2-ethynyl hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl (hydroxymethyl)tetrahydrofuran-3-ol (0.2 g, 0.682 mmol) in THF (40 mL) and pyridine (2.000 mL) was added tert-butylmagnesium chloride (1.432 mL, 1.432 mmol) dropwise at 0° C. Then, the reaction was stirred at 25° C. for 30 min. A solution of 2-hexyloctyl ((S)-(perfluorophenoxy)(phenoxy)phosphoryl)-L-phenylalaninate (0.606 g, 0.887 mmol) in THF (20 mL) was added dropwise to the above solution at −15° C. and stirred at −15° C. for 4 h. TLC showed starting material was consumed. The reaction was quenched with 2M HCl (20 mL) and extracted with EtOAc (30 mL×3). The combined organic phases were washed with a solution of NaHCO₃ (20 mL), brine (20 mL), dried with Na₂SO₄, filtered and concentrated. The residue was purified by silica gel chromatography (25 g, DCM/MeOH=100/0 to 10/1) to afford 2-hexyloctyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (210 mg, 0.254 mmol, 37.3% yield) as white solid. LCMS (M+H)=793; Retention time (0.1% TFA)=2.22 min. ¹H NMR (400 MHz, CDCl₃) δ 7.94 (s, 1H), 7.26-7.04 (m, 10H), 6.31 (dd, J=7.3, 4.4 Hz, 1H), 6.25-5.73 (m, 2H), 4.71 (s, 1H), 4.22 (dt, J=20.4, 11.5 Hz, 3H), 3.97 (d, J=5.7 Hz, 2H), 3.76 (t, J=10.9 Hz, 1H), 3.61-3.40 (m, 1H), 3.01 (d, J=6.2 Hz, 2H), 2.81-2.54 (m, 3H), 1.55 (s, 1H), 1.22 (s, 20H), 0.87 (t, J=6.9 Hz, 6H).

### Example 64

### 2-Heptylnonyl ((((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

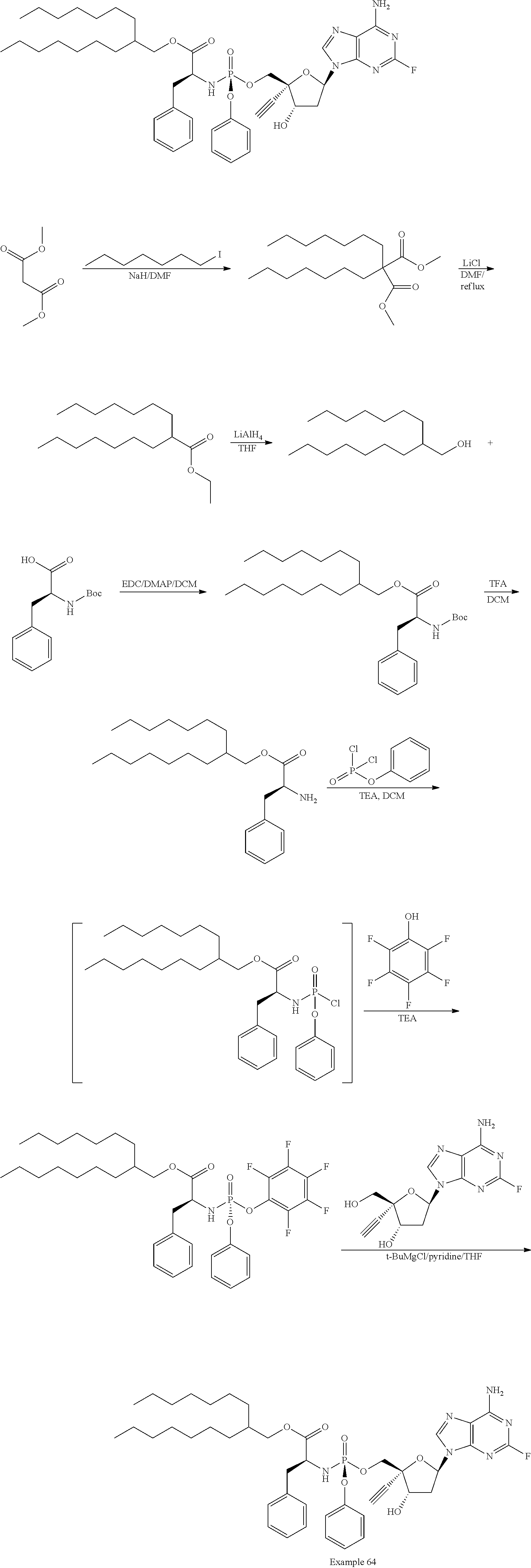

### Step 1: Dimethyl 2,2-diheptylmalonate

To a stirred suspension of sodium hydride (11.35 g, 284 mmol) in DMF (250 mL) was added dropwise dimethyl malonate (15 g, 114 mmol) at 0° C. under argon atmosphere. After 10 min, a solution of 1-iodoheptane (64.2 g, 284 mmol) in DMF (30 mL) was added and the resulting mixture was stirred at 25° C. for 3 h. LCMS showed the presence of desired product. The reaction mixture was quenched with water and extracted with EtOAc (2×30 mL). The combined organic layers were washed with water and brine, dried over MgSO₄, filtered and concentrated. The residue was purified by silica gel chromatography (EtOAc/hexane=0/100 to 2/98) to afford dimethyl 2,2-diheptylmalonate (9 g, 27.4 mmol, 24.13% yield) as colorless liquid. LCMS (M+H)=328; Retention time (0.1% TFA)=2.80 min.

### Step 2: Methyl 2-heptylnonanoate

A stirred mixture of the dimethyl 2,2-diheptylmalonate (25 g, 76 mmol), lithium chloride (6.45 g, 152 mmol) and water (0.78 mLin DMSO (75 mL) was heated at reflux for 6 h. Then, the reaction was cooled to room temperature and diluted with water (200 mL). The phases were separated and the aqueous layer was extracted with EtOAc (3×200 mL). The combined organic layers were washed with brine (200 mL) and dried over anhydrous Na₂SO₄, filtered, concentrated to give methyl 2-heptylnonanoate (20 g, 66.6 mmol, 87 yield) which was used in the next step without further purification. ¹H NMR (400 MHz, CDCl₃) δ 3.66 (d, J=8.7 Hz, 3H), 2.33 (tt, J=8.9, 5.4 Hz, 1H), 1.51-1.35 (m, 4H), 1.38-1.14 (m, 26H), 0.94-0.80 (m, 12H).

### Step 3: 2-Heptylnonan-1-ol

A mixture of methyl 2-heptylnonanoate (5 g, 18.49 mmol) and LiAlH₄ (37.0 mL, 37.0 mmol, 1M in THF) in THF (50 mL) was heated at reflux for 2 h. Then, cooled to 0° C. and carefully quenched with successive addition of water (1.5 mL), 15% aq. NaOH (1.5 mL) and water (3.9 mL). The solvent were removed under vacuum. The residue was dissolved in EtOAc (50 mL) and filtered. The resulting filtrate was washed with brine (200 mL), dried over Na₂SO₄, filtered and evaporated to dryness to afford 2-heptylnonan-1-ol (4.2 g, 15.59 mmol, 84% yield) as an yellow oil which was used in the next step without purification. 1H NMR (400 MHz, CDCl₃) δ 3.54 (d, J=5.5 Hz, 2H), 1.27 (s, 24H), 0.88 (t, J=6.8 Hz, 6H).

### Step 4: 2-Heptylnonyl (tert-butoxycarbonyl)-L-phenylalaninate

To a stirred mixture of (tert-butoxycarbonyl)-L-phenylalanine (5.47 g, 20.62 mmol) in DCM (100 mL) was added DMAP (0.252 g, 2.062 mmol), 2-heptylnonan-1-ol (5 g, 20.62 mmol) and EDC (5.14 g, 26.8 mmol) at 0° C. and stirred at room temperature for 2 h. TLC showed starting material was consumed. The reaction mixture was diluted with water (100 mL) and extracted with DCM (150 mL×3). The combined organic phases were washed with brine, dried over Na₂SO₄, filtered and concentrated to to give 2-heptylnonyl (tert-butoxycarbonyl)-L-phenylalaninate (8 g, 9.80 mmol, 47.5% yield) as an yellow oil which was used in the next step without purification.

### Step 5: 2-Heptylnonyl L-phenylalaninate

To a stirred solution of 2-heptylnonyl (tert-butoxycarbonyl)-L-phenylalaninate (5 g, 10.21 mmol) in DCM (75 mL was added TFA (7.87 mL, 102 mmol) at 0° C. and stirred at room temperature for 2 h. Then, the reaction mixture was concentrated and the pH of the residue was adjusted to 8-9 with sat. Na₂CO₃, extracted with DCM (80 mL×3). The combined organic phases were washed with brine (100 mL), dried over Na₂SO₄, filtered and concentrated. The residue was purified by silica gel chromatography (DCM:MeOH=100:0 to 20:1) to afford 2-heptylnonyl L-phenylalaninate (2.6 g, 6.34 mmol, 62.1% yield). ¹H NMR (400 MHz, CDCl₃) δ 7.36-7.13 (m, 5H), 4.05-3.95 (m, 2H), 3.79-3.63 (m, 1H), 3.08 (dd, J=13.5, 5.4 Hz, 1H), 2.91-2.80 (m, 1H), 1.58 (d, J=16.8 Hz, 3H), 1.33-1.20 (m, 24H), 0.88 (dd, J=8.2, 5.4 Hz, 6H).

### Step 6: 2-Heptylnonyl ((S)-(perfluorophenoxy)(phenoxy)phosphoryl)-L-phenylalaninate

To a stirred solution of 2-heptylnonyl L-phenylalaninate (8 g, 20.53 mmol) in anhydrous DCM (100 mL) was added triethylamine (2.86 mL, 20.53 mmol) at −70° C. over 15 min dropwise. To this mixture was added a solution of phenylphosphonic dichloride (4.00 g, 20.53 mmol) in anhydrous DCM (50 mL) over 1 h. The reaction mixture was stirred at this temperature for additional 30 min and then allowed to warm to 0° C. over 2 h and stirred for another 1 h. To this mixture was added a solution of 2,3,4,5,6-pentafluorophenol (3.78 g, 20.53 mmol and TEA (2.86 mL, 20.53 mmol) in DCM (30 mL) over 20 min and stirred at 0° C. for 16 h. The white solid (triethylamine hydrochloride) was filtered off and washed with DCM (25 mL). The filtrate was concentrated under reduced pressure, the residue was triturated with tert-butyl methyl ether (150 mL) and the triethylamine hydrochloride salt was removed by filtration. The cake was washed with tert-butyl methyl ether (2×25 mL) and the combined filtrates were concentrated under reduced pressure to give crude solid containing an even mixture of diastereomers. The diastereomers were triturated with hexanes (100 mL) and filtered to give 2-heptylnonyl ((S)-(perfluorophenoxy)(phenoxy)phosphoryl)-L-phenylalaninate (2.5 g, 3.16 mmol, 15.40 yield) as a white solid (>97% de as determined by ³¹PNMR). ¹H NMR (400 MHz, CDCl₃) δ 7.43-7.31 (m, 2H), 7.26-7.15 (m, 6H), 7.11-6.98 (m, 2H), 4.46 (ddd, J=15.7, 10.1, 5.8 Hz, 1H), 4.05-3.85 (m, 2H), 3.78 (t, J=11.1 Hz, 1H), 3.20-2.97 (m, 3H), 1.52 (d, J=16.7 Hz, 1H), 1.33-1.19 (m, 24H), 0.88 (t, J=6.7 Hz, 6H). ³¹PNMR (CDCl₃, 162 MHz) δ −1.54.

### Step 7: 2-Heptylnonyl ((S)-(((2R,3S,5R)-5-(6-amino-2-fluoro-9H-purin-9-yl)-2-ethynyl-3-hydroxytetrahydrofuran-2-yl)methoxy) (phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (20 mL) and pyridine (1.000 mL) was added tert-butylmagnesium chloride (2.148 mL, 2.148 mmol) dropwise at 0° C. and stirred at 25° C. for 30 min. A solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (20 mL) was added dropwise to the above solution at −15° C. and stirred at −15° C. for 12 h. LCMS showed the reaction was completed. The reaction was quenched with 2M HCl (10 mL) and extracted with EtOAc (20 mL×3). The combined organic layers were washed with a solution of NaHCO₃ (20 mL), brine (20 mL), dried over Na₂SO₄, filtered and concentrated in vacuum. The crude product was purified by the silica gel chromatography (25 g, DCM:MeOH=20:1) to afford 2-heptylnonyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (145 mg, 0.174 mmol, 16.97% yield). LCMS: Retention time (0.1% TFA)=3.03 min. ¹H NMR (400 MHz, CDCl₃) δ 7.90 (s, 1H), 7.25-6.98 (m, 10H), 6.31 (dd, J=7.1, 4.5 Hz, 3H), 4.73 (t, J=7.6 Hz, 1H), 4.38-4.15 (m, 3H), 4.03-3.88 (m, 4H), 3.00 (d, J=6.2 Hz, 2H), 2.75-2.60 (m, 3H), 1.53 (s, 1H), 1.35-1.10 (m, 24H), 0.95-0.83 (m, 6H).

### Example 65

### 2-Octyldecyl ((S)-(((2R,3S,5R)-5-(6-amino-2-fluoro-9H-purin-9-yl)-2-ethynyl hydroxytetrahydrofuran-2-yl)methoxy)(phenoxy)phosphoryl)-L-phenylalaninate

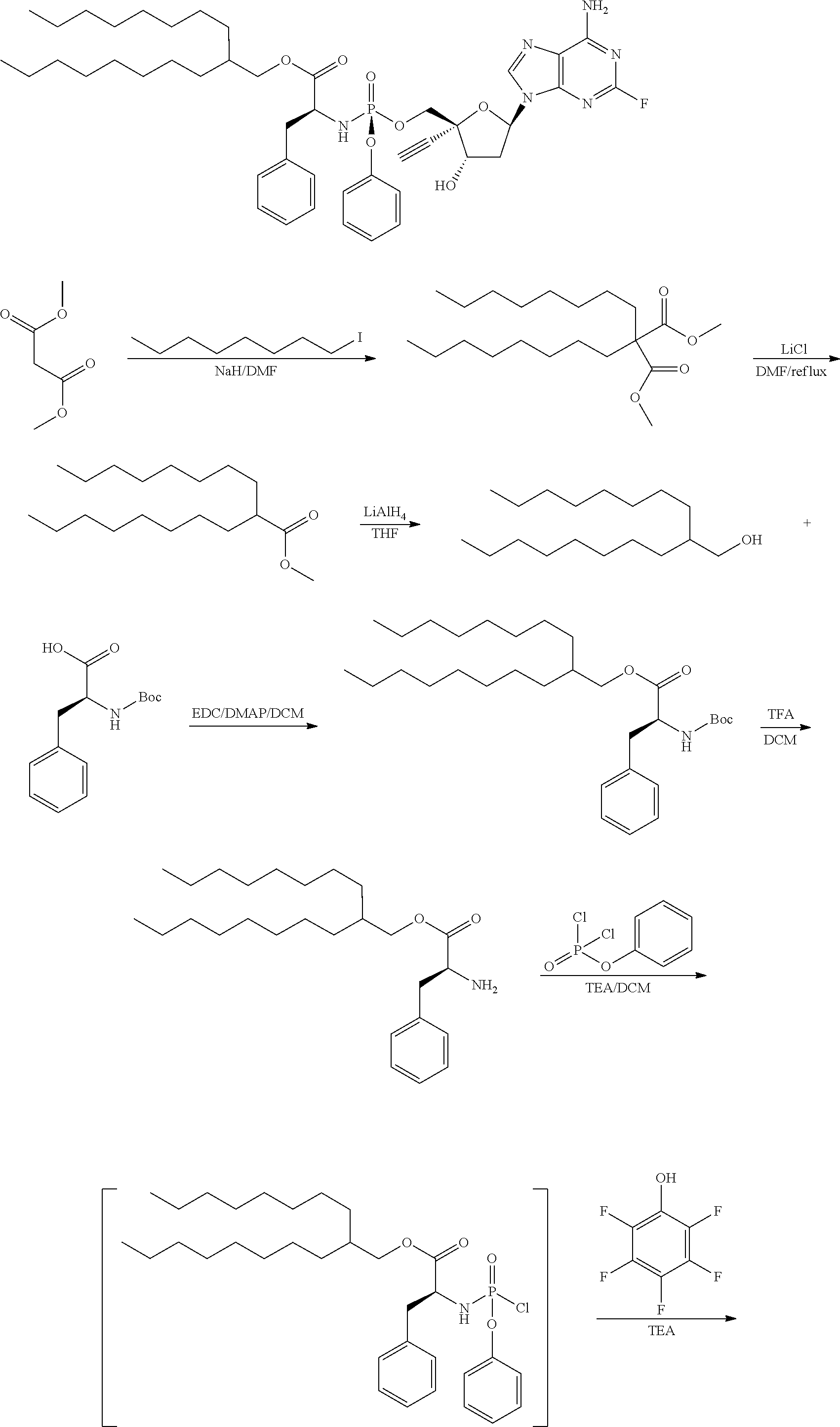

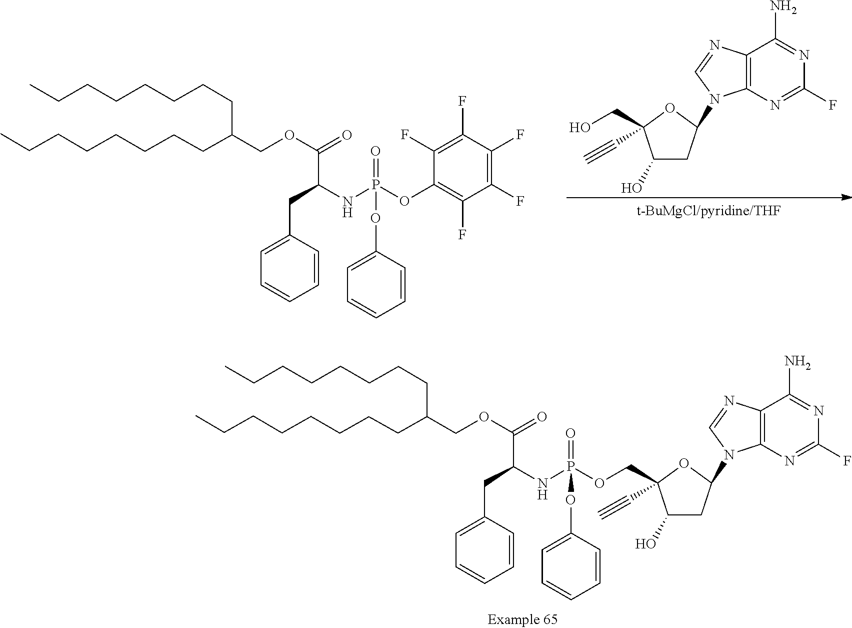 [SMILES: CCCCCCCCC(CCCCCCCC)COC(=O)[C@H](Cc1ccccc1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2ccccc2)C(=O)OCC(CCCCCCCC)CCCCCCCC)Oc2ccccc2)O[C@@H](n2cnc3c(N)nc(F)nc32)C[C@@H]1O]

### Step 1: Dimethyl 2,2-dioctylmalonate

To a stirred suspension of sodium hydride (12.87 g, 322 mmol) in DMF (30 mL) was added dropwise dimethyl malonate (14.71 mL, 129 mmol) at 0° C. under argon atmosphere. After 10 min, a solution of 1-iodooctane (77 g, 322 mmol) in DMF (30 mL) was added to the mixture and the resulting mixture was stirred at room temperature for 3 h. LCMS showed the presence of desired product. The reaction mixture was quenched with water and extracted with EtOAc (2×20 mL). The combined organic layers were washed with water and brine, dried over MgSO₄, filtered and evaporated. The residue was purified by silica gel chromatography (EtOAc/hexanes=0/100 to 2/98) to afford dimethyl 2,2-dioctylmalonate (42 g, 106 mmol, 82% yield) as yellow oil. LCMS (M+H)=356; Retention time (0.1% TFA)=2.767 min.

### Step 2: Methyl 2-octyldecanoate

A mixture of dimethyl 2,2-dioctylmalonate (12 g, 33.7 mmol), lithium chloride (2.85 g, 67.3 mmol) and water (0.68 mL) in DMSO (50 mL) was stirred at reflux for 6 h. LCMS showed the presence of desired product. The reaction mixture was diluted with water (80 mL) and extracted with EtOAc (200 mL×2). The combined organic phases were washed with brine, dried over Na₂SO₄, filtered and concentrated. The residue was purified by silica gel chromatography (100% pet. ether) to afford methyl 2-octyldecanoate (11.16 g, 33.7 mmol, 100% yield). LCMS (M+H)=299; Retention time (0.1% TFA)=3.85 min.

### Step 3: 2-Octyldecan-1-ol

A mixture of methyl 2-octyldecanoate (40.5 g, 136 mmol) and LiAlH₄ (228 mL, 228 mmol, 1M in THF) in THF (50 mL) was heated at reflux for 1 h. TLC showed the reaction was completed. The reaction mixture was cooled to 0° C. and quenched with successive addition of water (1.5 mL), 15% aq. NaOH (1.5 mL) and water (3.9 mL). The resulting mixture was filtered and the filtrate was dried over Na₂SO₄, filtered and evaporated to dryness to afford 2-octyldecan-1-ol (26 g, 96 mmol, 70.8% yield) which was used in the next step without further purification. ¹HNMR (400 MHz, CDCl₃) δ 3.59-3.44 (m, 2H), 1.27 (s, 29H), 0.94-0.84 (m, 6H).

### Step 4: 2-Octyldecyl (tert-butoxycarbonyl)-L-phenylalaninate

To a stirred mixture of (tert-butoxycarbonyl)-L-phenylalanine (5.88 g, 22.18 mmol) and EDC (5.53 g, 28.8 mmol) in DCM (150 mL) was added DMAP (0.271 g, 2.218 mmol) at 0° C. After 30 min, 2-octyldecan-1-ol (6.0 g, 22.18 mmol) was added and stirred at 25° C. for 16 h. TLC showed the reaction was completed. The reaction mixture was diluted with water (80 mL) and extracted with DCM (100 mL×2). The combined organic phases were washed with brine, dried over Na₂SO₄ and concentrated. The residue was purified by silica gel chromatography (pet. ether:EtOAc=10:1) to afford 2-octyldecyl (tert-butoxycarbonyl)-L-phenylalaninate (8.1 g, 14.86 mmol, 67.0% yield) as a yellow oil. δ 7.30-7.22 (m, 3H), 7.12 (d, J=1.6 Hz, 2H), 4.98 (t, J=4.0 Hz, 1H), 4.58 (m, 1H), 4.00-3.96 (m, 2H), 3.07 (qt, J=31.7, 15.8 Hz, 2H), 1.41 (s, 9H), 1.26 (dd, J=22.6, 5.5 Hz, 28H), 0.88 (t, J=6.8 Hz, 6H).

### Step 5: 2-Octyldecyl L-phenylalaninate

To a stirred solution of 2-octyldecyl (tert-butoxycarbonyl)-L-phenylalaninate (8.1 g, 15.64 mmol) in DCM (75 mL) was added TFA (20 mL, 260 mmol) at 0° C. and the mixture was stirred at room temperature for 2 h. Then, the reaction mixture was concentrated and the pH of the residue was adjusted to 8-9 with sat. Na₂CO₃, and extracted with DCM (80 mL×3). The combined organic phases were washed with brine (100 mL), dried over Na₂SO₄, filtered and concentrated. The residue was purified by silica gel chromatography (DCM:MeOH=100:0 to 20:1) to afford 2-octyldecyl L-phenylalaninate (5.0 g, 11.97 mmol, 77% yield) as a white solid. δ 7.31-7.18 (m, 5H), 4.01-3.99 (m, 2H), 3.72 (dt, J=25.8, 12.9 Hz, 1H), 3.06 (dd, J=13.5, 5.4 Hz, 1H), 2.88 (dd, J=13.5, 7.8 Hz, 1H), 1.59-1.53 (br, 2H), 1.28 (d, J=17.6 Hz, 28H), 0.88 (t, J=6.7 Hz, 6H).

### Step 6: 2-Octyldecyl ((S)-(perfluorophenoxy)(phenoxy)phosphoryl)-L-phenylalaninate

To a stirred solution of 2-octyldecyl L-phenylalaninate (5.0 g, 11.97 mmol) in anhydrous DCM (50 mL) was added triethylamine (1.830 mL, 13.17 mmol) at −70° C. over 15 min dropwise. To this mixture was added a solution of phenyl phosphorodichloridate (2.5 g, 11.85 mmol) in anhydrous DCM (30 mL) over 1 h at −70° C. The reaction mixture was stirred at this temperature for additional 30 min and then allowed to warm to 0° C. over 2 h and stirred for another 1 h. To this mixture was added a solution of 2,3,4,5,6-pentafluorophenol (2.181 g, 11.85 mmol) and triethylamine (1.830 mL, 13.17 mmol) in DCM (20 mL) over 20 min and stirred at 0° C. for 4 h. TLC showed the reaction was completed. The white solid (triethylamine hydrochloride) was filtered off and washed with DCM (50 mL). The filtrate was concentrated under reduced pressure and the residue was triturated with tert-butyl methyl ether (150 mL). Then, the triethylamine hydrochloride salt was removed by filtration. The cake was washed with tert-butyl methyl ether (2×50 mL) and the combined filtrates were concentrated. The crude product was triturated with hexanes (50 mL) and filtered to give 2-octyldecyl ((S)-(perfluorophenoxy)(phenoxy)phosphoryl)-L-phenylalaninate (1.1 g, 1.487 mmol, 12.42% yield) as a white solid (>98% de as determined by ³¹PNMR). ¹H NMR (400 MHz, CDCl3) δ 7.35 (t, J=7.9 Hz, 2H), 7.25-7.16 (m, 6H), 7.04 (dd, J=6.9, 2.4 Hz, 2H), 4.46 (t, J=5.6 Hz, 1H), 3.96 (ddd, J=33.0, 10.8, 5.7 Hz, 2H), 3.82-3.68 (m, 1H), 3.10 (ddd, J=20.0, 13.8, 5.8 Hz, 2H), 1.54 (s, 1H), 1.26 (t, J=15.2 Hz, 28H), 0.88 (t, J=6.7 Hz, 6H). ³¹PNMR (CDCl₃, 162 MHz) δ −1.53.

### Step 7: 2-Octyldecyl ((S)-(((2R,3S,5R)-5-(6-amino-2-fluoro-9H-purin-9-yl)-2-ethynyl-3-hydroxytetrahydrofuran-2-yl)methoxy) (phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (40 mL) and pyridine (2.000 mL) was added tert-butylmagnesium chloride (2.148 mL, 2.148 mmol) dropwise at 0° C. Then, the reaction was stirred at 25° C. for 30 min. A solution of 2-octyldecyl ((S)-(perfluorophenoxy)(phenoxy)phosphoryl)-L-phenylalaninate (1.060 g, 1.432 mmol) in THF (20 mL) was added dropwise to the above solution at −15° C. and stirred at −15° C. for 4 h. TLC showed starting material was consumed. The reaction was quenched with 2M HCl (20 mL) and extracted with EtOAc (30 mL×3). The combined organic phases were washed with a solution of NaHCO₃ (20 mL), brine (20 mL), dried with Na₂SO₄, filtered and concentrated. The residue was purified by silica gel chromatography (25 g, DCM/MeOH=100/0 to 10/1) to afford 2-octyldecyl ((S)-(((2R,3S,5R)-5-(6-amino-2-fluoro-9H-purin-9-yl) ethynyl-3-hydroxytetrahydrofuran-2-yl)methoxy)(phenoxy)phosphoryl)-L-phenylalaninate (175 mg, 0.200 mmol, 19.54% yield) as white solid. LCMS (M+Na)=871; Retention time (0.05% TFA)=2.715 min. ¹H NMR (400 MHz, MeOD) δ 8.12 (s, 1H), 7.36-6.98 (m, 10H), 6.31 (dd, J=7.6, 4.3 Hz, 1H), 4.68 (t, J=7.5 Hz, 1H), 4.17-4.05 (m, 2H), 3.98 (dd, J=11.0, 5.3 Hz, 1H), 3.93-3.80 (m, 2H), 3.18 (s, 1H), 3.01 (s, 1H), 2.90-2.81 (m, 1H), 2.74 (s, 1H), 2.67-2.56 (m, 1H), 1.49 (s, 1H), 1.25 (t, J=15.8 Hz, 28H), 0.89 (t, J=6.9 Hz, 6H).

### Example 66

### 2-Nonylundecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

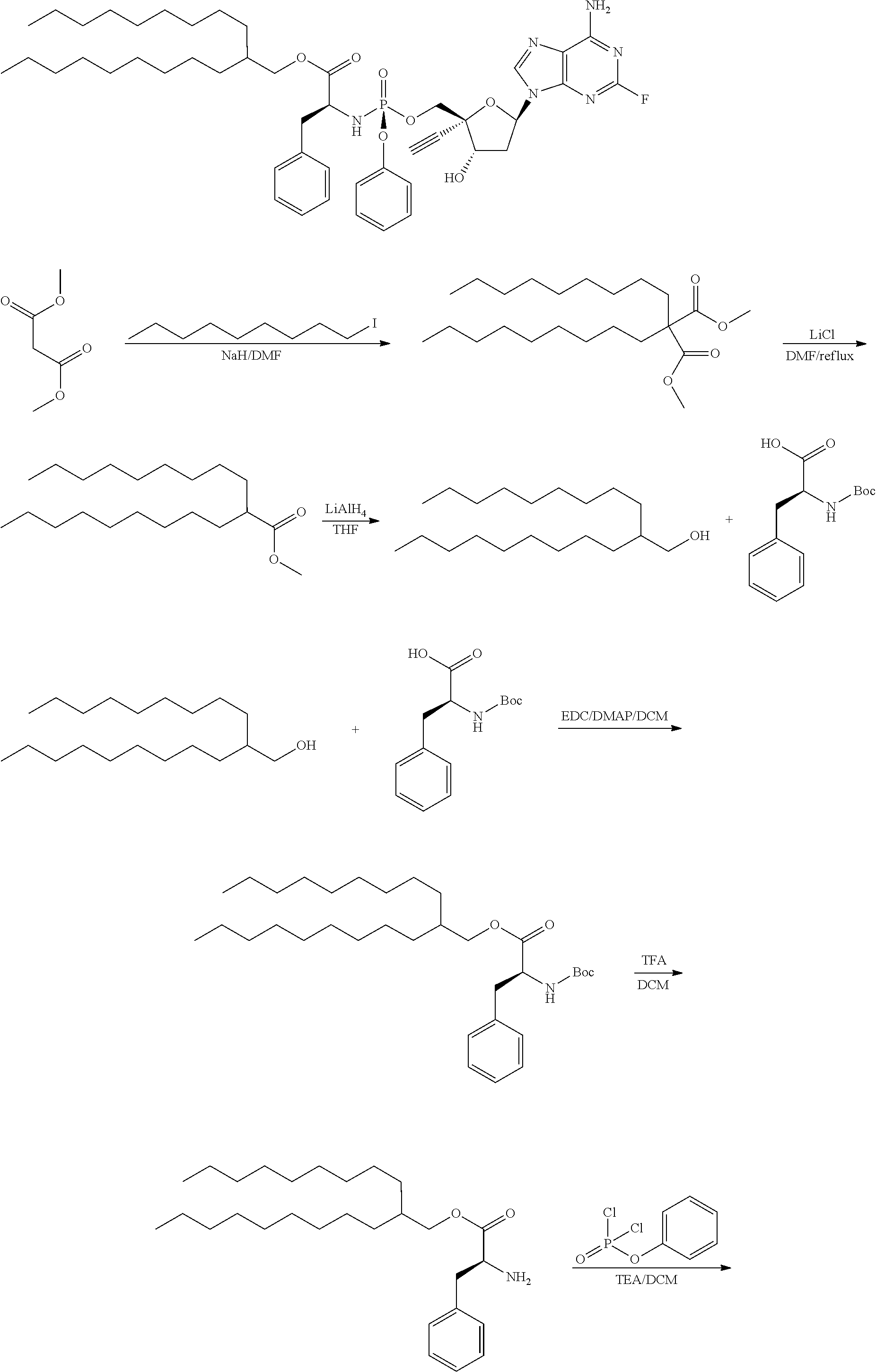

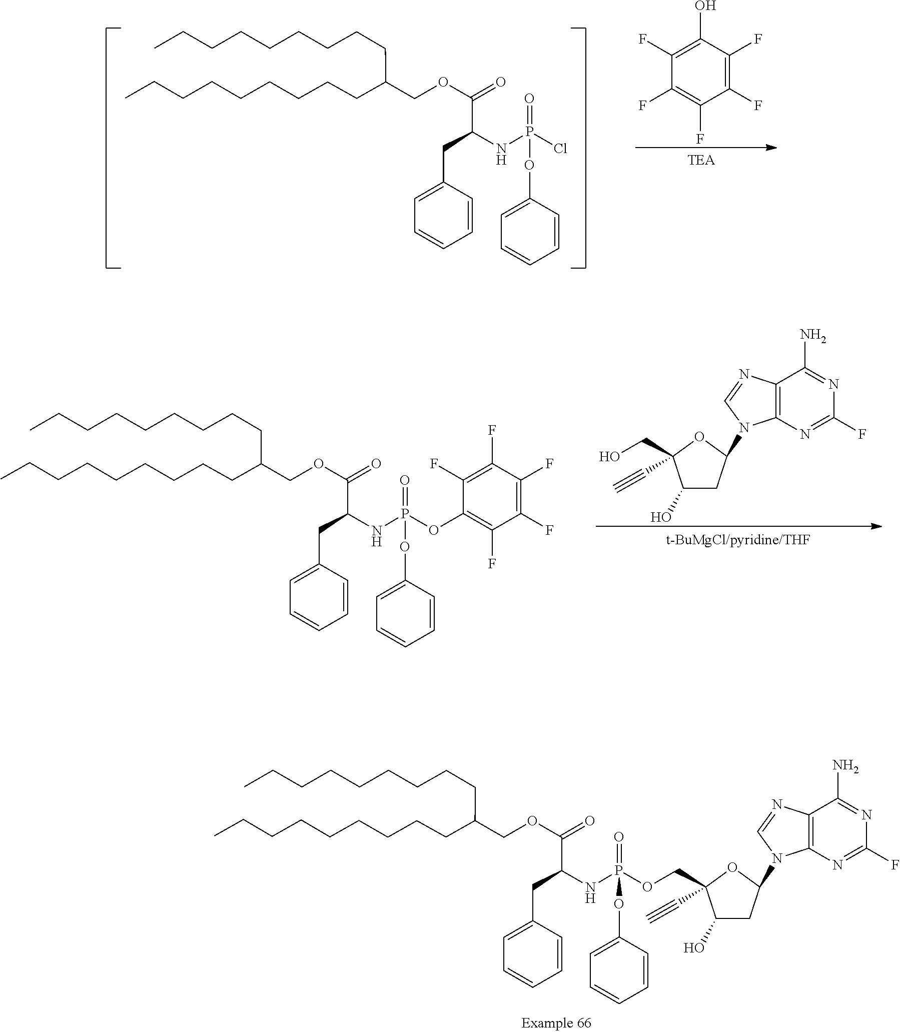

### Step 1: Dimethyl 2,2-dinonylmalonate

To a stirred suspension of sodium hydride (7.66 g, 191 mmol) in DMF (120 mL) was added dimethyl malonate (10 g, 76 mmol) dropwise at 0° C. under argon atmosphere. After 10 min, a solution of 1-iodononane (48.7 g, 191 mmol) in DMF (75 mL) was added to the mixture, and the resulting mixture was stirred at room temperature for 3 h. LCMS showed the reaction was completed. The reaction mixture was quenched with water (100 mL) and extracted with EtOAc (150 mL×2). The combined organic layers were washed with water and brine (100 mL), dried over MgSO₄, filtered and evaporated under vacuum to give dimethyl 2,2-dinonylmalonate (27 g, 70.2 mmol) which was used in the next step without further purification. LCMS (M+H)=385.4, Retention time (0.1% TFA): 3.75 min.

### Step 2: Methyl 2-nonylundecanoate

A stirred mixture of dimethyl 2,2-dinonylmalonate (27 g, 70.2 mmol), H₂O (1.265 mL, 70.2 mmol) and lithium chloride (5.95 g, 140 mmol) in DMSO (110 mL) was heated at 190° C. under N₂ atmosphere for 8 h. LCMS showed the reaction was completed. The reaction was cooled, diluted with EtOAc (150 mL) and H₂O (200 mL). The mixture was stirred vigorously for 30 min, and the organic phase was washed with sat NaCl (100 mL×2), dried over Na₂SO₄ and concentrated to give methyl 2-nonylundecanoate (21 g, 64.3 mmol, 92% yield) as an oil which was used in the next step without further purification. LCMS (M+H)=327.4, Retention time (0.1% TFA): 4.371 min.

### Step 3: 2-Nonylundecan-1-ol

To a stirred solution of methyl 2-nonylundecanoate (21 g, 64.3 mmol) in THF (150 mL) was added 1M LiAlH₄/THF (129 mL, 129 mmol) dropwise at room temperature and the mixture was stirred at 70° C. for 1 h. TLC (pet. ether:EtOAc=50:1, Rf=0.9) showed there was a new compound. After cooling to 0° C., the reaction mixture was quenched with successive addition of water (5 mL), 15% aq. NaOH (5 mL), water (15 mL) and Na₂SO₄ (60 g). The resulting mixture was diluted with THF and the solid was removed by filtration. The filtrate was evaporated under reduced pressure to give 2-nonylundecan-1-ol (20 g, 60.3 mmol, 94% yield) as oil was used in the next step without further purification. ¹H NMR (400 MHz, CDCl₃) δ 3.53 (d, J=5.4 Hz, 2H), 1.49-1.40 (m, 1H), 1.26 (s, 32H), 0.88 (t, J=6.8 Hz, 6H).

### Step 4: 2-Nonylundecyl (tert-butoxycarbonyl)-L-phenylalaninate

To a stirred mixture of (tert-butoxycarbonyl)-L-phenylalanine (8.89 g, 33.5 mmol) and EDC (8.35 g, 43.5 mmol) in DCM (150 mL) was added DMAP (0.409 g, 3.35 mmol) at 0° C. After 30 min, 2-nonylundecan-1-ol (10 g, 33.5 mmol) and the resulting reaction mixture was stirred at 25° C. for 16 h. TLC showed the reaction was completed. The reaction mixture was diluted with water (80 mL) and extracted with DCM (100 mL×2). The combined organic phases were washed with brine, dried over Na₂SO₄ and concentrated. The residue was purified by silica gel chromatography (pet. ether:EtOAc=10:1) to afford 2-nonylundecyl (tert-butoxycarbonyl)-L-phenylalaninate (16 g, 29.3 mmol, 88% yield) as an yellow oil. ¹H NMR (400 MHz, CDCl₃) δ 7.28-7.22 (m, 3H), 7.13 (d, J=1.6 Hz, 2H), 4.98 (t, J=4.0 Hz, 1H), 4.57 (m, 1H), 4.00-3.96 (m, 2H), 3.01 (qt, J=31.7, 15.8 Hz, 2H), 1.41 (s, 9H), 1.26 (dd, J=22.6, 5.5 Hz, 32H), 0.88 (t, J=6.8 Hz, 6H).

### Step 5: 2-Nonylundecyl L-phenylalaninate

To a stirred solution of 2-nonylundecyl (tert-butoxycarbonyl)-L-phenylalaninate (17.1 g, 31.3 mmol) in DCM (90 mL) was added TFA (45 mL, 584 mmol) at 0° C. and the mixture was stirred at room temperature for 2 h. Then, the reaction mixture was concentrated and the pH of the residue was adjusted to 8-9 with sat. Na₂CO₃, and extracted with DCM (80 mL×3). The combined organic phases were washed with brine (100 mL), dried over Na₂SO₄, filtered and concentrated. The residue was purified by silica gel chromatography (DCM: MeOH=100:0 to 20:1) to afford 2-nonylundecyl L-phenylalaninate (8.5 g, 19.07 mmol, 60.9% yield) as yellow oil. ¹H NMR (400 MHz, CDCl₃) δ 7.30 (t, J=7.2 Hz, 2H), 7.26-7.13 (m, 3H), 4.21-3.90 (m, 2H), 3.84-3.59 (m, 1H), 3.21-3.01 (m, 1H), 2.97-2.82 (m, 1H), 1.60 (s, 1H), 1.51 (s, 2H), 1.28 (d, J=15.1 Hz, 32H), 0.88 (t, J=6.8 Hz, 6H).

### Step 6: 2-Nonylundecyl ((S)-(perfluorophenoxy)(phenoxy)phosphoryl)-L-phenylalaninate

To a stirred solution of 2-nonylundecyl L-phenylalaninate (8 g, 17.95 mmol) in anhydrous DCM (100 mL) was added triethylamine (2.502 mL, 17.95 mmol) at −70° C. over 15 min dropwise. To this mixture was added a solution of phenylphosphonic dichloride (3.50 g, 17.95 mmol) in anhydrous DCM (50 mL) over 1 h at −70° C. The reaction mixture was stirred at this temperature for additional 30 min and then allowed to warm to 0° C. over 2 h and stirred for another 1 h. To this mixture was added a solution of 2,3,4,5,6-pentafluorophenol (3.30 g, 17.95 mmol) and TEA (2.502 mL, 17.95 mmol) in DCM (30 mL) over 20 min and stirred at 0° C. for 16 h. LCMS showed the reaction was completed. The white solid (triethylamine hydrochloride) was filtered off and washed with DCM (25 mL). The filtrate was concentrated under reduced pressure and the residue was triturated with tert-butyl methyl ether (150 mL). Then, the triethylamine hydrochloride salt was removed by filtration. The cake was washed with tert-butyl methyl ether (2×25 mL) and the combined filtrates were concentrated under reduced pressure to give crude solid containing an even mixture of diastereomers. The mixture was triturated with hexanes (15 mL) and filtered to afford 2-nonylundecyl ((S)-(perfluorophenoxy)(phenoxy)phosphoryl)-L-phenylalaninate (1.5 g, 1.856 mmol, 10.34% yield) as a white solid (>98% de as determined by ³¹PNMR). ¹H NMR (400 MHz, CDCl₃) δ 7.35 (t, J=7.8 Hz, 2H), 7.26-7.15 (m, 6H), 7.03 (dd, J=13.7, 9.1 Hz, 2H), 4.45 (ddd, J=15.6, 10.1, 5.8 Hz, 1H), 4.09-3.89 (m, 2H), 3.86-3.75 (m, 1H), 3.22-2.99 (m, 2H), 1.54 (s, 1H), 1.27 (dd, J=27.8, 9.6 Hz, 32H), 0.88 (t, J=6.7 Hz, 6H). ³¹PNMR (CDCl₃, 162 MHz) δ −1.53.

### Step 7: 2-Nonylundecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate

To a stirred solution of (2R,3S,5R)-5-(6-amino-2-fluoro-9H-purin-9-yl)-2-ethynyl-2-(hydroxymethyl)tetrahydrofuran-3-ol (0.3 g, 1.023 mmol) in THF (20 mL) and pyridine (1.00 mL) was added tert-butylmagnesium chloride (1.074 mL, 1.074 mmol) dropwise at 0° C. and stirred at 25° C. for 30 min. A solution of 2-nonylundecyl ((S)-(perfluorophenoxy)(phenoxy)phosphoryl)-L-phenylalaninate (0.943 g, 1.228 mmol) in THF (20 mL) was added dropwise to the above solution at −15° C. Then, the reaction was stirred at −15° C. for 12 h. LCMS showed the reaction was completed. The reaction was quenched with 2M HCl (10 mL) and extracted with EtOAc (20 mL×3). The combined organic layers were washed with a solution of NaHCO₃ (20 mL), brine (20 mL), dried over Na₂SO₄, filtered and concentrated in vacuum. The crude product was purified by the silica gel chromatography (25 g, MeOH/DCM=0-7%) to give 2-nonylundecyl ((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)-L-phenylalaninate (110 mg, 0.125 mmol, 12.19%). LCMS: Retention time (0.1% TFA)=3.73 min. ¹H NMR (400 MHz, CDCl₃) δ 7.91 (s, 1H), 7.24-7.02 (m, 10H), 6.31 (dd, J=7.2, 4.4 Hz, 1H), 6.06 (s, 2H), 4.71 (t, J=7.3 Hz, 1H), 4.36-4.10 (m, 3H), 3.96 (d, J=5.7 Hz, 2H), 3.83 (t, J=10.9 Hz, 1H), 3.61 (s, 1H), 3.15-2.91 (m, 2H), 2.78-2.60 (m, 3H), 1.54 (s, 1H), 1.34-1.14 (m, 32H), 0.87 (t, J=6.8 Hz, 6H).

### Example 67

### 2-Butylhexyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

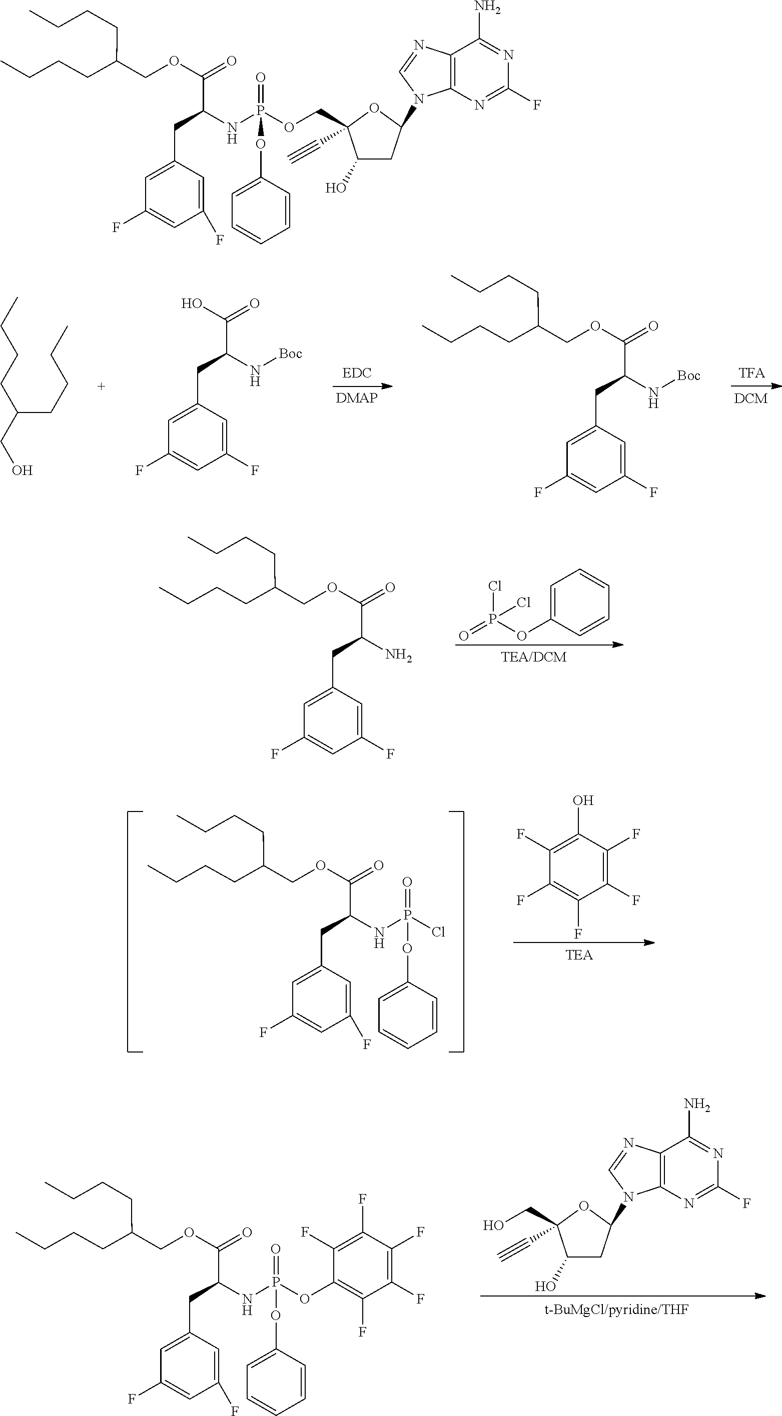

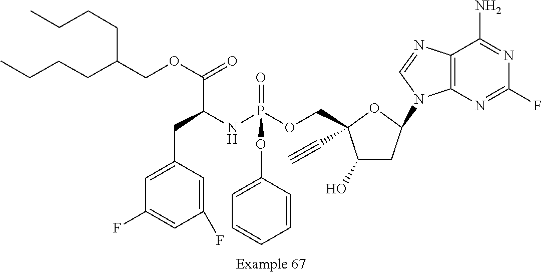 [SMILES: C#C[C@]1(CO[P@@](=O)(N[C@@H](Cc2cc(F)cc(F)c2)C(=O)OCC(CCCC)CCCC)Oc2ccccc2)O[C@@H](n2cnc3c(N)nc(F)nc32)C[C@@H]1O]

### Step 1: 2-Butylhexyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (4 g, 13.28 mmol) in DCM (60 mL) were added DMAP (0.162 g, 1.328 mmol) and EDC (3.31 g, 17.26 mmol) at 0° C. The resulting reaction mixture was stirred at the same temperature for 30 min, then 2-butylhexan-1-ol (2.52 g, 15.93 mmol) was added. The resulting reaction mixture was stirred at 25° C. for 16 h. LCMS showed the reaction was completed. The reaction was diluted with water (50 mL) and extracted with DCM (100 mL×2). The combined organic layers were washed with brine (40 mL), dried over Na₂SO₄, filtered and concentrated to afford 2-butylhexyl (S)-2-((tert-butoxycarbonyl)amino)-3-(3,5-difluorophenyl)propanoate (4.6 g, 10.42 mmol, 78% yield) as a white solid which was used in the next step without further purification. LCMS: (M+Na)=464.0; Retention time (0.1% TFA)=2.59 min.

### Step 2: 2-Butylhexyl (S)-2-amino-3-(3,5-difluorophenyl)propanoate

To a stirred solution of 2-butylhexyl (S)-2-((tert-butoxycarbonyl)amino)-3-(3,5-difluorophenyl)propanoate (4.6 g, 10.42 mmol) in DCM (60 mL) was added TFA (15 mL, 195 mmol) dropwise at 0° C. The resulting reaction mixture was stirred at 25° C. for 16 h. LCMS showed the reaction was completed. The reaction mixture was concentrated and the residue was neutralized with NaHCO₃ (40 mL) and extracted with DCM (3×80 mL). The combined organic layers were washed with brine (40 mL), dried over Na₂SO₄, filtered and concentrated. The residue was purified by silica gel chromatography (petroleum ether/ethyl acetate=2/1) to give 2-butylhexyl (S)-2-amino-3-(3,5-difluorophenyl)propanoate (3.3 g, 9.66 mmol, 93% yield) as a light yellow solid. LCMS (M+H)=342.1; Retention time (0.1% TFA)=1.76 min; purity: 100% (254 nm).

### Step 3: 2-Butylhexyl (S)-3-(3,5-difluorophenyl)-2-((S)-(perfluorophenoxy)(phenoxy)phosphoryl)amino)propanoate

To a stirred solution of 2-butylhexyl (S)-2-amino-3-(3,5-difluorophenyl)propanoate (3.3 g, 9.66 mmol) in DCM (60 mL) was added triethylamine (1.478 mL, 10.63 mmol) dropwise at −70° C. Then to this mixture was added a solution of phenyl phosphorodichloridate (2.019 g, 9.57 mmol) in anhydrous DCM (10 mL) dropwise. The reaction mixture was stirred at this temperature for additional 30 min and then allowed to warm to 0° C. over 2 h and stirred for 1 h. To this mixture was added a solution of 2,3,4,5,6-pentafluorophenol (1.779 g, 9.66 mmol) and triethylamine (1.478 mL, 10.63 mmol) in DCM (15 mL) dropwise and stirred at 0° C. for 4 h. LCMS showed the reaction was completed. The mixture was concentrated and the residue was triturated with tert-butyl methyl ether (120 mL) and the triethylamine hydrochloride salt was removed by filtration. The cake was washed with tert-butyl methyl ether (2×30 mL) and the combined filtrates were concentrated under reduced pressure. The residue was triturated with 10% EtOAc in hexanes (40 mL) and solids were collected by filtration to obtain 2-butylhexyl (S)-3-(3,5-difluorophenyl)-2-(((S)-(perfluorophenoxy)(phenoxy)phosphoryl)amino)propanoate (1.4 g, 2.110 mmol, 21.83% yield) as a white solid (>98% de as determined by ³¹PNMR). LCMS (M+H)=664.9; Retention time (0.1% TFA)=2.65 min. ³¹PNMR (CDCl₃, 162 MHz) δ −1.55.

### Step 4: 2-Butylhexyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl (hydroxymethyl)tetrahydrofuran-3-ol (200 mg, 0.682 mmol) in THF (40 mL) and pyridine (10 mL) was added tert-butylmagnesium chloride (1.364 mL, 1.364 mmol) dropwise at −15° C. The reaction mixture was stirred at the same temperature for 1 h. Then, to the reaction mixture was added a solution of 2-butylhexyl (S)-3-(3,5-difluorophenyl)-2-(((S)-(perfluorophenoxy)(phenoxy)phosphoryl)amino)propanoate (634 mg, 0.955 mmol) in anhydrous THF (20 mL) dropwise and stirred at −15° C. for 4 h. LCMS showed the reaction was completed. The reaction mixture was quenched with 2N NH₄C₁ (10 mL) and partitioned between EtOAc (200 mL) and H₂O (30 mL). The organic layer was washed with brine (30 mL), dried over Na₂SO₄, filtered and concentrated. The residue was purified by reverse phase chromatography (SepaFlash® C18 column, BOSTON, 40 g, 0-100 CH₃CN/10 mM NH₄HCO₃ H₂O) to obtain 2-butylhexyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (133 mg, 0.164 mmol, 24.02% yield) as a white solid. LCMS (M+H)=772.9; Retention time (10 mM NH₄HCO₃)=2.22 min; purity: 100% (254 nm). HPLC Retention time (10 mM NH₄HCO₃)=11.32 min. ¹H NMR (400 MHz, CDCl₃) δ 7.94 (s, 1H), 7.32-7.29 (m, 2H), 7.19-7.12 (m, 3H), 6.68-6.62 (m, 3H), 6.34 (dd, J=7.6, 4.4 Hz, 1H), 6.03 (brs, 2H), 4.81 (dd, J=12.4, 7.2 Hz, 1H), 4.41-4.25 (m, 3H), 4.03-3.95 (m, 3H), 3.59 (d, J=5.2 Hz, 1H), 2.99 (d, J=6.0 Hz, 2H), 2.85 (ddd, J=13.6, 7.2, 4.0 Hz, 1H), 2.75 (s, 1H), 2.74-2.67 (m, 1H), 1.60-1.51 (m, 1H), 1.29-1.20 (m, 12H), 0.88 (t, J=6.8 Hz, 6H).

### Example 68

### 2-Heptylnonyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

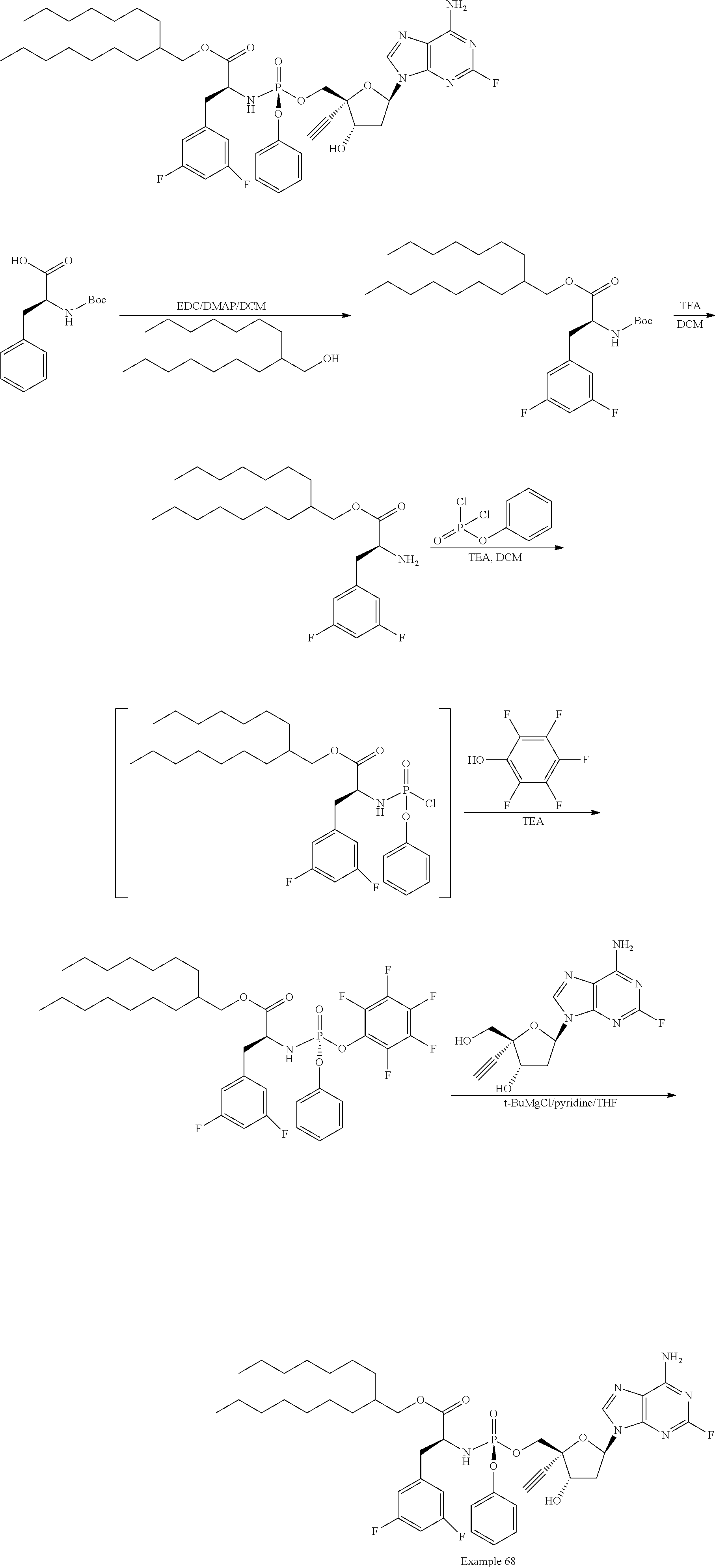

### Step 1: 2-Heptylnonyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (10 g, 33.2 mmol) in DCM (120 mL) were added DMAP (0.405 g, 3.32 mmol) and EDC (8.27 g, 43.1 mmol) at 0° C. and stirred at the same temperature for 30 min. Then, to this mixture was added a solution of 2-heptylnonan-1-ol (9.66 g, 39.8 mmol) in DCM (10 mL) and stirred at 25° C. for 16 h. LCMS showed the reaction was completed. The reaction mixture was diluted with water (50 mL) and extracted with DCM (200 mL×3). The combined organic layers were washed with brine (50 mL), dried over Na₂SO₄, filtered and concentrated. The residue was purified by silica gel chromatography (petroleum ether/ethyl acetate=20/1) to give 2-heptylnonyl (S)-2-((tert-butoxycarbonyl)amino)-3-(3,5-difluorophenyl)propanoate (12.5 g, 22.13 mmol, 66.7% yield) as a white solid. LCMS: (M+Na)=548.2; Retention time (0.05% TFA)=3.83 min; purity: 93.08% (214 nm).

### Step 2: 2-Heptylnonyl (S)-2-amino-3-(3,5-difluorophenyl)propanoate

To a stirred solution of 2-heptylnonyl (S)-2-((tert-butoxycarbonyl)amino)-3-(3,5-difluorophenyl)propanoate (12.5 g, 23.78 mmol) in DCM (90 mL) was added TFA (20 mL, 260 mmol) dropwise at 0° C. and stirred at 25° C. for 4 h. LCMS showed the reaction was completed. The reaction mixture was concentrated and the residue was neutralized with NaHCO₃ (50 mL). The mixture was partitioned between water (50 mL) and DCM (300 mL). The aqueous layer was extracted with DCM (2×200 mL). The combined organic layers were washed with brine (60 mL), dried over Na₂SO₄, filtered and concentrated. The residue was purified by silica gel chromatography (petroleum ether/ethyl acetate=2/1) to give 2-heptylnonyl (S)-2-amino-3-(3,5-difluorophenyl)propanoate (7 g, 15.98 mmol, 67.2 yield) as a light yellow oil. LCMS (M+H)=426.4; Retention time (0.05% TFA)=2.12 min; purity: 97.15% (214 nm).

### Step 3: 2-Heptylnonyl (S)-3-(3,5-difluorophenyl)-2-((S)-(perfluorophenoxy)(phenoxy)phosphoryl)amino)propanoate

To a stirred solution of 2-heptylnonyl (S)-2-amino-3-(3,5-difluorophenyl)propanoate (7 g, 16.45 mmol) in DCM (100 mL) was added triethylamine (2.51 mL, 18.09 mmol) dropwise at −70° C. To this mixture was added a solution of phenyl phosphorodichloridate (3.44 g, 16.28 mmol) in anhydrous DCM (20 mL) dropwise. The reaction mixture was stirred at this temperature for additional 30 min and then allowed to warm to 0° C. over 2 h and stirred for 1 h. To this mixture was added a solution of 2,3,4,5,6-pentafluorophenol (3.03 g, 16.45 mmol) and triethylamine (2.51 mL, 18.09 mmol) in DCM (20 mL) dropwise. The mixture was allowed to stirred at 0° C. for 16 h. LCMS showed the reaction was completed. The reaction mixture was concentrated under reduced pressure and the residue was triturated with tert-butyl methyl ether (200 mL), and the triethylamine hydrochloride salt was removed by filtration. The cake was washed with tert-butyl methyl ether (2×30 mL) and the combined filtrate was concentrated under reduced pressure. The mixture was triturated with 1% EtOAc in hexanes (50 mL) and solids collected by filtration. The filtrate was concentrated and the residue was triturated with 1% EtOAc in hexanes (25 mL) and solids collected by filtration. This procedure was repeated twice to give 2-heptylnonyl (S)-3-(3,5-difluorophenyl)-2-(((S)-(perfluorophenoxy)(phenoxy)phosphoryl)amino)propanoate (1.2 g, 1.527 mmol, 9.28% yield) as a white solid (>98% de as determined by ³¹PNMR). LCMS (M+H)=748.0; Retention time (0.1% TFA)=3.85 min. ³¹PNMR (CDCl₃, 162 MHz) δ −1.55.

### Step 4: 2-Heptylnonyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (200 mg, 0.682 mmol) in THF (40.00 mL) and pyridine (10 mL) was added tert-butylmagnesium chloride (1.364 mL, 1.364 mmol) dropwise at −15° C. The reaction mixture was stirred at the same temperature for 1 h. Then, to this reaction mixture was added a solution of 2-heptylnonyl (S)-3-(3,5-difluorophenyl)-2-(((S)-(perfluorophenoxy)(phenoxy)phosphoryl)amino)propanoate (663 mg, 0.887 mmol) in anhydrous THF (10 mL) dropwise. The reaction mixture was stirred at −15° C. for 3 h. LCMS showed the reaction was completed. The reaction mixture was quenched with 2N NH₄Cl (10 mL) and partitioned between EtOAc (200 mL) and H₂O (30 mL). The organic layer was washed with brine (30 mL), dried over Na₂SO₄, filtered and concentrated. The residue was purified by reverse phase chromatography (SepaFlash® C18 column, BOSTON, 40 g, 0-100% MeOH/10 mM NH₄CO₃ H₂O) to give 2-heptylnonyl (S)-2-(((S)-(((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate (189 mg, 0.214 mmol, 31.4% yield) as a white solid. LCMS (M+H)=857.0; Retention time (0.1% TFA)=2.70 min; purity: 100% (254 nm). HPLC Retention time (10 mM NH₄CO₃)=7.41 min. ¹H NMR (400 MHz, CDCl₃) δ 7.94 (s, 1H), 7.29-7.24 (m, 2H), 7.16-7.10 (m, 3H), 6.66-6.59 (m, 3H), 6.32 (dd, J=7.6, 4.4 Hz, 1H), 6.18 (brs, 2H), 4.80 (t, J=7.6 Hz, 1H), 4.39-4.22 (m, 3H), 4.16 (t, J=10.4 Hz, 1H), 3.94 (d, J=5.6 Hz, 2H), 3.70 (brs, 1H), 2.97 (d, J=6.4 Hz, 2H), 2.80 (ddd, J=13.6, 7.6, 4.4 Hz, 1H), 2.73 (s, 1H), 2.71-2.65 (m, 1H), 1.53 (brs, 1H), 1.26-1.18 (m, 24H), 0.87 (t, J=6.8 Hz, 6H).

### Example 69

### 2-Octyldecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

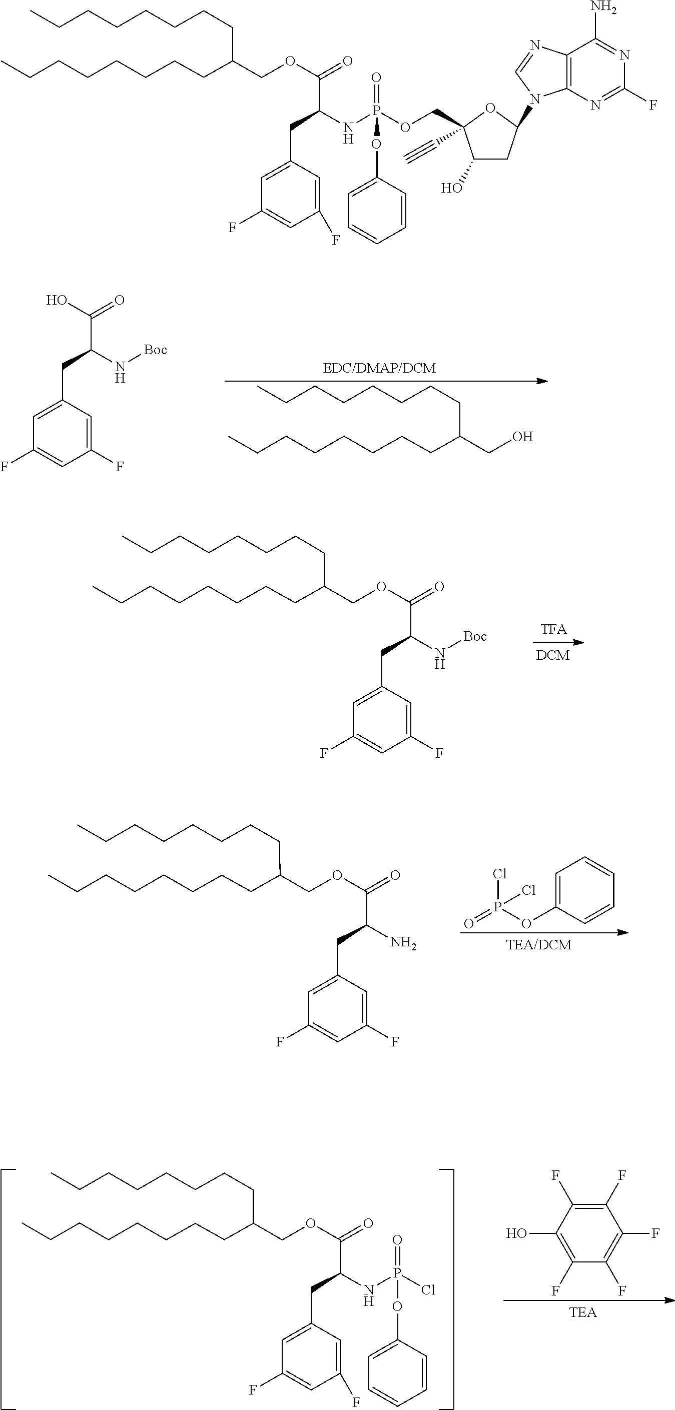

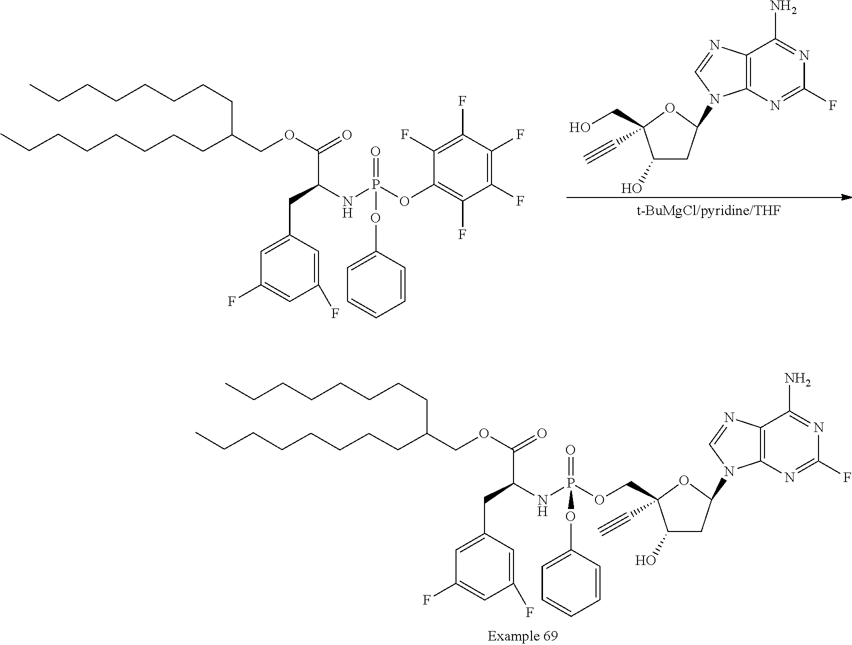 [SMILES: CCCCCCCCC(CCCCCCCC)COC(=O)[C@H](Cc1cc(F)cc(F)c1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2cc(F)cc(F)c2)C(=O)OCC(CCCCCCCC)CCCCCCCC)Oc2ccccc2)O[C@@H](n2cnc3c(N)nc(F)nc32)C[C@@H]1O]

### Step 1: 2-Octyldecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (10 g, 33.2 mmol) in DCM (120 mL) were added DMAP (0.405 g, 3.32 mmol) and EDC (8.27 g, 43.1 mmol) at 0° C. and stirred at the same temperature for 30 min. Then, to this mixture was added 2-octyldecan-1-ol (10.77 g, 39.8 mmol) in DCM (10 mL) and stirred at 25° C. for 16 h. LCMS showed the reaction was completed. The reaction mixture was diluted with water (50 mL) and extracted with DCM (200 mL×2). The combined organic layers were washed with brine (50 mL), dried over Na₂SO₄, filtered and concentrated. The residue was purified by silica gel chromatography (petroleum ether/ethyl acetate=20/1) to give 2-octyldecyl (S)-2-((tert-butoxycarbonyl)amino)-3-(3,5-difluorophenyl)propanoate (13.5 g, 20.11 mmol, 60.6% yield) as a white solid. LCMS: (M+Na)=576.2; Retention time (0.1% TFA)=4.81 min; purity: 82.50% (214 nm).

### Step 2: 2-Octyldecyl (S)-2-amino-3-(3,5-difluorophenyl)propanoate

To a stirred solution of 2-octyldecyl (S)-2-((tert-butoxycarbonyl)amino)-3-(3,5-difluorophenyl)propanoate (13.5 g, 24.38 mmol) in DCM (100 mL) was added TFA (20 mL, 260 mmol) dropwise at 0° C. and stirred at 25° C. for 16 h. LCMS showed the reaction was completed. The reaction was concentrated and the residue was neutralized with NaHCO₃ (50 mL) and extracted with DCM (3×200 mL). The combined organic layers were washed with brine (60 mL), dried over Na₂SO₄, filtered and concentrated. The residue was purified by silica gel chromatography (petroleum ether/ethyl acetate=2/1) to give 2-octyldecyl (S)-2-amino-3-(3,5-difluorophenyl)propanoate (7.1 g, 15.65 mmol, 64.2% yield) as a light yellow oil. LCMS (M+H)=454.3; Retention time (0.05% TFA)=2.35 min; purity: 100 (254 nm).

### Step 3: 2-Octyldecyl (S)-3-(3,5-difluorophenyl)-2-((S)-(perfluorophenoxy)(phenoxy)phosphoryl)amino)propanoate

To a stirred solution of 2-octyldecyl (S)-2-amino-3-(3,5-difluorophenyl)propanoate (7.1 g, 15.65 mmol) in DCM (100 mL) was added triethylamine (2.393 mL, 17.22 mmol) dropwise at −70° C. To this mixture was added a solution of phenyl phosphorodichloridate (3.27 g, 15.49 mmol) in anhydrous DCM (20 mL) dropwise. The reaction mixture was stirred at this temperature for additional 30 min and then allowed to warm to 0° C. over 2 h and stirred for 1 h. To this mixture was added a solution of 2,3,4,5,6-pentafluorophenol (2.88 g, 15.65 mmol) and triethylamine (2.393 mL, 17.22 mmol) in DCM (20 mL) dropwise. The crude mixture was allowed to stirred at 0° C. for 16 h. LCMS showed the reaction was completed. The reaction mixture was concentrated under reduced pressure and the residue was triturated with tert-butyl methyl ether (200 mL), and the triethylamine hydrochloride salt was removed by filtration. The cake was washed with tert-butyl methyl ether (2×30 mL) and the combined filtrate was concentrated under reduced pressure. The mixture was triturated with hexanes (50 mL) and solids collected by filtration. The filtrate was concentrated and the residue was triturated with hexanes (25 mL) and solids collected by filtration. This procedure was repeated twice to give 2-octyldecyl (S)-3-(3,5-difluorophenyl)-2-(((S)-(perfluorophenoxy)(phenoxy)phosphoryl)amino)propanoate (1.5 g, 1.570 mmol, 10.03% yield) as a white solid (>98% de as determined by ³¹PNMR). LCMS (M+H)=776.0; Retention time (0.1% TFA)=4.75 min. ³¹PNMR (CDCl₃, 162 MHz) δ −1.56.

### Step 4: 2-Octyldecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl (hydroxymethyl)tetrahydrofuran-3-ol (250 mg, 0.852 mmol) in THF (50.00 mL) and pyridine (12 mL) was added tert-butylmagnesium chloride (1.705 mL, 1.705 mmol) dropwise at −15° C. The reaction mixture was stirred at the same temperature for 1 h. Then, to this reaction mixture was added a solution of 2-octyldecyl (S)-3-(3,5-difluorophenyl)-2-(((S)-(perfluorophenoxy)(phenoxy)phosphoryl)amino)propanoate (860 mg, 1.108 mmol) in anhydrous THF (15 mL) dropwise. The reaction mixture was stirred at −15° C. for 3 h. LCMS showed the reaction was completed. The reaction mixture was quenched with 2N NH₄Cl (10 mL) and partitioned between EtOAc (200 mL) and H₂O (40 mL). The organic layer was washed with brine (40 mL), dried over Na₂SO₄, filtered and concentrated. The residue was purified by reverse phase chromatography (SepaFlash® C18 column, BOSTON, 40 g, 0-100% MeOH/10 mM NH₄HCO₃ H₂O) to give 2-octyldecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (265 mg, 0.290 mmol, 34.0% yield) as a white solid. LCMS (M+H)=885.1; Retention time (0.1% TFA)=3.01 min; purity: 100% (254 nm). HPLC Retention time (10 mM NH₄HCO₃)=8.68 min. ¹H NMR (400 MHz, CDCl₃) δ 7.98 (s, 1H), 7.30-7.27 (m, 2H), 7.16-7.10 (m, 3H), 6.67-6.60 (m, 3H), 6.32 (dd, J=7.6, 4.4 Hz, 1H), 5.99 (brs, 2H), 4.78 (t, J=7.6 Hz, 1H), 4.37-4.24 (m, 3H), 3.96 (d, J=5.6 Hz, 2H), 3.93 (t, J=6.4 Hz, 1H), 2.99 (d, J=6.0 Hz, 2H), 2.82 (ddd, J=13.6, 6.8, 4.4 Hz, 1H), 2.49 (brs, 1H), 2.73 (s, 1H), 2.72-2.65 (m, 1H), 1.55 (brs, 1H), 1.27-1.20 (m, 28H), 0.87 (t, J=6.4 Hz, 6H).

### Example 70

### 2-Pentylheptyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

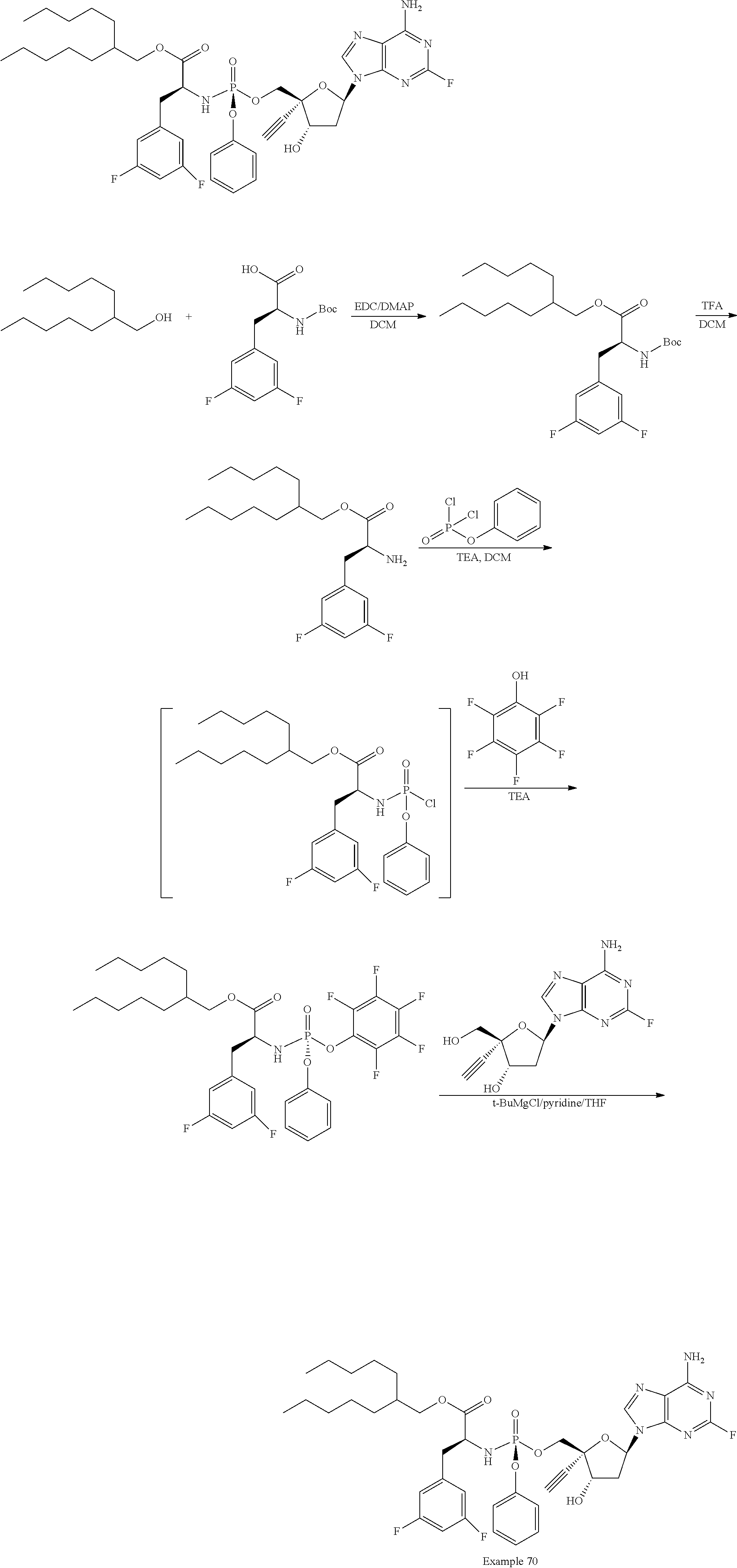

### Step 1: 2-Pentylheptyl (S)-2-((tert-butoxycarbonyl)amino)-3-(3,5-difluorophenyl)propanoate

To a stirred mixture of (S)-2-((tert-butoxycarbonyl)amino)-3-(3,5-difluorophenyl)propanoic acid (8.71 g, 28.9 mmol) in DCM (50 mL) was added EDC (7.22 g, 37.6 mmol) and DMAP (0.353 g, 2.89 mmol) at 0° C. After 30 min, 2-pentylheptan-1-ol (7.00 g, 37.6 mmol) in DCM (20 mL) was added at 0° C. and the reaction was stirred at 15° C. for 16 h. LCMS showed the product was detected. The reaction mixture was diluted with water (30 mL) and extracted with DCM (25 mL×2). The combined organic phases were washed with brine, dried over Na₂SO₄ and concentrated to give 2-pentylheptyl (S)-2-((tert-butoxycarbonyl)amino)-3-(3,5-difluorophenyl)propanoate (11.2 g, 7.99 mmol, 27.6% yield) as yellow oil which was used in the next step without further purification. LCMS (2M+Na)=961.2; Retention time (10% TFA)=2.854 min.

### Step 2: 2-Pentylheptyl (S)-2-amino-3-(3,5-difluorophenyl)propanoate

To a stirred mixture of 2-pentylheptyl (S)-2-((tert-butoxycarbonyl)amino)-3-(3,5-difluorophenyl)propanoate (11.2 g, 23.85 mmol) in DCM (60 mL) was added TFA (18.06 mL, 234 mmol) at 0° C. and it was stirred at 15° C. overnight. LCMS showed the reaction was completed. The reaction was concentrated and the pH of the residue was adjusted to 8-9 with sat. Na₂CO₃. The mixture was partitioned, and the aqueous phase was extracted with DCM (50 mL×2). The combined organic phases were washed with sat. Na₂CO₃, dried over Na₂SO₄ and concentrated. The residue was purified by silica gel chromatography (Santai, 120 g, DCM:MeOH=20:1) to afford 2-pentylheptyl (S)-2-amino-3-(3,5-difluorophenyl)propanoate (7.15 g, 13.57 mmol, 56.9% yield) as light yellow oil. LCMS (M+H)=370.1; Retention time (0.1% TFA)=1.932 min. ¹H NMR (400 MHz, CDCl₃) δ 6.79 (d, J=5.7 Hz, 2H), 6.72 (td, J=8.9, 2.1 Hz, 1H), 4.19 (t, J=6.8 Hz, 1H), 3.99 (t, J=5.6 Hz, 2H), 3.23 (ddd, J=21.6, 14.3, 6.8 Hz, 2H), 1.53 (d, J=5.3 Hz, 1H), 1.30-1.14 (m, 16H), 0.87 (t, J=7.0 Hz, 6H).

### Step 3: 2-Pentylheptyl (S)-3-(3,5-difluorophenyl)-2-(((S)-(perfluorophenoxy)(phenoxy)-phosphoryl)amino)propanoate

To a stirred solution of 2-pentylheptyl (S)-2-amino-3-(3,5-difluorophenyl)propanoate (7.15 g, 19.35 mmol) in anhydrous DCM (50 mL) was added triethylamine (2.82 mL, 20.32 mmol) at −70° C. over 15 min dropwise. To this mixture was added a solution of phenyl phosphorodichloridate (4.04 g, 19.16 mmol) in anhydrous DCM (20 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (3.53 g, 19.16 mmol) and triethylamine (2.96 mL, 21.29 mmol) in DCM (20 mL) over 20 min and stirred at 0° C. for 3 h. LCMS showed the presence of desired product. The white solid (triethylamine hydrochloride) was filtered off and washed with DCM (1×25 mL). The filtrate was concentrated under reduced pressure. The residue was triturated with tert-butyl methyl ether (200 mL) and the triethylamine hydrochloride salt was removed by filtration. The cake was washed with tert-butyl methyl ether (2×25 mL) and the combined filtrate was concentrated under reduced pressure. The crude solid was triturated in pet. ether (80 mL), filtered and washed with pet. ether (2×15 mL) to give 2-pentylheptyl (S)-3-(3,5-difluorophenyl)-2-(((S)-(perfluorophenoxy)(phenoxy)phosphoryl)amino)propanoate (1.3 g, 1.406 mmol, 7.26 yield) as white solid (>95% de as determined by ³¹PNMR). LCMS (M+H)=692.0, Retention time (0.1% NH₄HCO₃)=2.89 min. ³¹PNMR (CDCl₃, 162 MHz) δ −1.58.

### Step 4: 2-Pentylheptyl (S)-2-((S)-(((2R,3S,5R)-5-(6-amino-2-fluoro-9H-purin-9-yl)-2-ethynyl-3-hydroxytetrahydrofuran-2-yl)methoxy)(phenoxy) phosphoryl)amino)-3-(3,5-di fluorophenyl) propanoate

To a stirred solution of (2R,3S,5R)-5-(6-amino-2-fluoro-9H-purin-9-yl)-2-ethynyl-2-(hydroxymethyl)tetrahydrofuran-3-ol (0.22 g, 0.750 mmol) in THF (40 mL) and pyridine (10 mL) was added tert-butylmagnesium chloride (1.575 mL, 1.575 mmol) drop-wise at −15° C. After 30 min, a solution of 2-pentylheptyl (S)-3-(3,5-difluorophenyl)-2-(((S)-(perfluorophenoxy)(phenoxy)phosphoryl)amino)propanoate (0.675 g, 0.975 mmol) in THF (10 mL) was added dropwise to the above solution at −15° C. and as stirred at −15° C. for 16 h. LCMS showed the reaction was completed. The reaction was quenched with sat. NH₄Cl (20 mL) and extracted with EtOAc (20 mL×3). The combined organic layers were washed with brine (20 mL), dried with Na₂SO₄, filtered and concentrated in vacuum. The crude product was purified by reverse chromatography (SepaFlash® C18 column, BOSTON, 40 g, 0-100% CH₃CN/10 mM NH₄HCO₃ H₂O) to give 2-pentylheptyl (S)-2-(((S)-(((2R,3S,5R)-5-(6-amino-2-fluoro-9H-purin-9-yl)-2-ethynyl-3-hydroxytetrahydrofuran yl)methoxy)(phenoxy)phosphoryl)amino)-3-(3,5-difluorophenyl)propanoate (166 mg, 0.199 mmol, 26.5% yield) as white solid. LCMS (M+H)=801.3, Retention time (0.1% TFA): 2.065 min; HPLC: Retention time (10 mM NH₄CO₃)=12.055 min; ¹H NMR (400 MHz, CDCl₃) δ 8.85 (s, 1H), 7.26-7.25 (m, 1H), 7.23 (d, J=7.7 Hz, 2H), 7.09 (t, J=9.3 Hz, 3H), 6.69 (d, J=5.9 Hz, 2H), 6.57 (t, J=8.9 Hz, 1H), 6.36 (dd, J=6.4, 3.8 Hz, 1H), 4.80 (s, 1H), 4.70 (t, J=7.5 Hz, 1H), 4.42-4.30 (m, 2H), 4.24-4.14 (m, 1H), 3.94 (d, J=5.6 Hz, 2H), 3.06-2.94 (m, 2H), 2.82-2.69 (m, 3H), 1.54 (s, 1H), 1.30-1.24 (m, 4H), 1.21 (s, 12H), 0.86 (t, J=7.0 Hz, 6H).

### Example 71

### 2-Nonylundecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

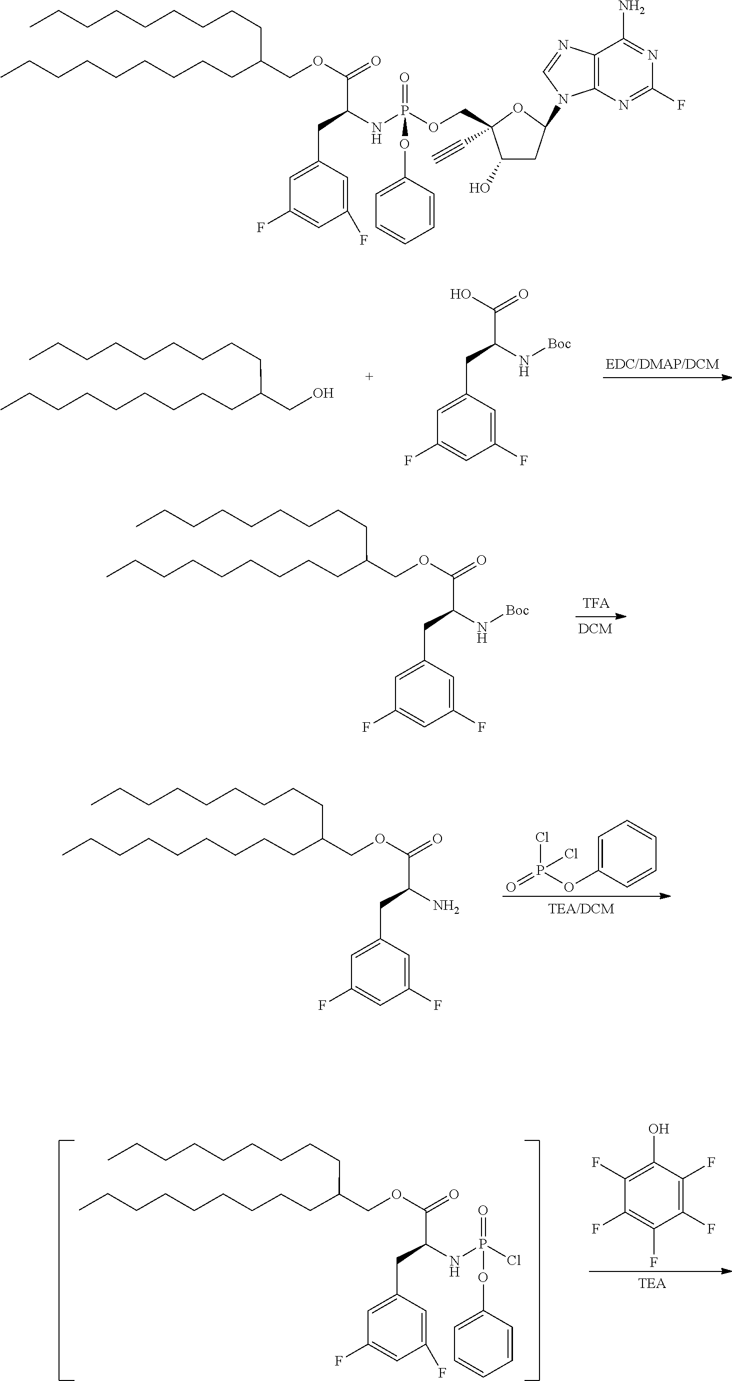

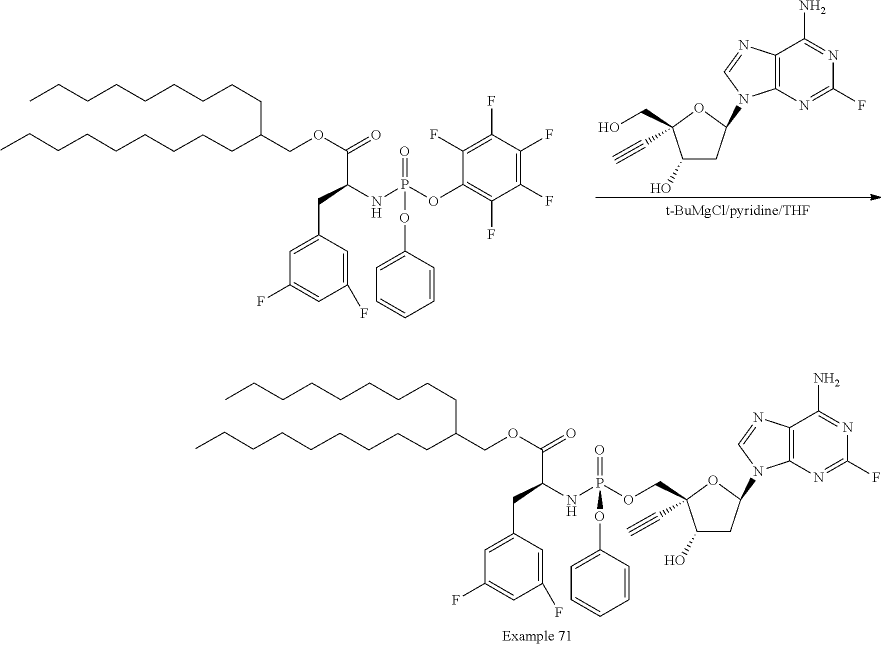 [SMILES: CCCCCCCCCC(CCCCCCCCC)COC(=O)[C@H](Cc1cc(F)cc(F)c1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2cc(F)cc(F)c2)C(=O)OCC(CCCCCCCCC)CCCCCCCCC)Oc2ccccc2)O[C@@H](n2cnc3c(N)nc(F)nc32)C[C@@H]1O]

### Step 1: 2-Nonylundecyl (S)-2-((tert-butoxycarbonyl)amino)-3-(3,5-difluorophenyl)propanoate

To a stirred solution of (S)-2-((tert-butoxycarbonyl)amino)-3-(3,5-difluorophenyl)propanoic acid (6 g, 19.91 mmol) in DCM (60 mL) was added N,N-dimethylpyridin-4-amine (0.243 g, 1.991 mmol) and 3-(((ethylimino)methylene)amino)-N,N-dimethylpropan-1-amine hydrochloride (4.97 g, 25.9 mmol) at 0° C. After 30 min, a solution of 2-nonylundecan-1-ol (7.73 g, 25.9 mmol) in DCM (15 mL) was added dropwise to the above solution at 0° C. and stirred overnight at 15° C. Then, the reaction mixture was diluted with water (30 mL) and partitioned. Aqueous phase was extracted with DCM (35 mL×2) and the combined organic phases were washed with brine (20 mL), dried with Na₂SO₄, filtered and concentrated in vacuum to give 2-nonylundecyl (S)-2-((tert-butoxycarbonyl)amino)-3-(3,5-difluorophenyl)propanoate (14.06 g, 24.17 mmol, 121% yield) as an yellow oil which was used in the next step without purification.

### Step 2: 2-Nonylundecyl (S)-2-amino-3-(3,5-difluorophenyl)propanoate

To a stirred mixture of 2-nonylundecyl (S)-2-((tert-butoxycarbonyl)amino)-3-(3,5-difluorophenyl)propanoate (16.26 g, 27.9 mmol) in DCM (60 mL) was added TFA (21.16 mL, 275 mmol) at 0° C. and stirred at 15° C. overnight. LCMS showed the reaction was completed. The reaction was concentrated and the pH of the residue was adjusted to 8-9 with sat. Na₂CO₃. The mixture was extracted with DCM (50 mL×2). The combined organic phases were washed with sat. Na₂CO₃, dried over Na₂SO₄ and concentrated. The residue was purified by silica gel chromatography (Santai, 120 g, DCM:MeOH=50:1) to afford 2-nonylundecyl (S)-2-amino-3-(3,5-difluorophenyl)propanoate (4.5 g, 7.88 mmol, 28.2% yield) as light yellow oil. LCMS (M+H)=482.4; Retention time (0.1% TFA)=2.838 min. ¹H NMR (400 MHz, CDCl₃) δ 6.74 (d, J=6.2 Hz, 2H), 6.69 (td, J=9.0, 2.2 Hz, 1H), 4.00 (d, J=5.7 Hz, 2H), 3.71 (dd, J=7.4, 5.8 Hz, 1H), 3.04 (dd, J=13.6, 5.6 Hz, 1H), 2.85 (dd, J=13.6, 7.6 Hz, 1H), 1.29 (s, 1H), 1.25 (s, 32H), 0.87 (t, J=6.7 Hz, 6H).

### Step 3: 2-Nonylundecyl (S)-3-(3,5-difluorophenyl)-2-(((S)-(perfluorophenoxy)(phenoxy)-phosphoryl)amino)propanoate

To a stirred solution of 2-nonylundecyl (S)-2-amino-3-(3,5-difluorophenyl)propanoate (4.5 g, 9.34 mmol) in anhydrous DCM (50 mL) was added triethylamine (1.363 mL, 9.81 mmol) dropwise at −70° C. over 15 min. To this mixture was added a solution of phenyl phosphorodichloridate (1.951 g, 9.25 mmol) in anhydrous DCM (15 mL) over 1 h. The reaction mixture was stirred at this temperature for additional 30 min and then allowed to warm to 0° C. over 2 h and stirred for 1 h. To this mixture was added a solution of 2,3,4,5,6-pentafluorophenol (1.702 g, 9.25 mmol) and triethylamine (1.428 mL, 10.28 mmol) in DCM (15 mL) over 20 min and stirred at 0° C. for 3 h. LCMS showed the presence of desired product. The white solid (triethylamine hydrochloride) was filtered off and washed with DCM (1×25 mL). The filtrate was concentrated under reduced pressure. The residue was triturated with tert-butyl methyl ether (150 mL) and the triethylamine hydrochloride salt was removed by filtration. The cake was washed with tert-butyl methyl ether (2×25 mL) and the combined filtrate was concentrated under reduced pressure. The crude solid was triturated in pet. ether (5 mL) and stirred for 16 h. The mixture was filtered to give 2-nonylundecyl (S)-3-(3,5-difluorophenyl)-2-(((S)-(perfluorophenoxy)(phenoxy)phosphoryl)amino)propanoate (550 mg, 0.684 mmol, 7.32 yield) as white solid (>95% de as determined by ³¹PNMR). ¹H NMR (400 MHz, CDCl₃) δ 7.36 (t, J=7.5 Hz, 2H), 7.28 (s, 1H), 7.22 (t, J=7.2 Hz, 3H), 6.67 (t, J=8.7 Hz, 1H), 6.57 (d, J=6.1 Hz, 2H), 4.44 (s, 1H), 4.05 (dd, J=10.7, 5.4 Hz, 1H), 3.96 (dd, J=10.4, 5.5 Hz, 1H), 3.88 (t, J=10.4 Hz, 1H), 1.57 (s, 1H), 1.32 (d, J=6.2 Hz, 2H), 1.25 (s, 30H), 0.87 (d, J=6.7 Hz, 6H). ³¹PNMR (CDCl₃, 162 MHz) δ −1.59.

### Step 4: 2-Nonylundecyl (S)-2-((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (0.154 g, 0.525 mmol) in THF (30 mL) and pyridine (7.50 mL) was added tert-butylmagnesium chloride (1.103 mL, 1.103 mmol) dropwise at −15° C. After 30 min, a solution of 2-nonylundecyl (S)-3-(3,5-difluorophenyl)-2-(((S)-(perfluorophenoxy)(phenoxy)phosphoryl)amino)propanoate (0.549 g, 0.683 mmol) in THF (7.5 mL) was added dropwise to the above solution at −15° C. and stirred at −15° C. for 4 h. LCMS showed the reaction was completed. The reaction mixture was quenched with sat. NH₄Cl (20 mL) ad extracted with EtOAc (20 mL×3). The combined organic layers were washed with brine (20 mL), dried with Na₂SO₄, filtered and concentrated in vacuum. The crude product was purified by reverse chromatography column (SepaFlash® C18 column, BOSTON, 40 g, 0-100% CH₃CN/10 mM NH₄HCO₃ H₂O) to give 2-nonylundecyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate (175 mg, 0.187 mmol, 35.6% yield) as white solid. LCMS (M+H)=913.5, Retention time (0.1% TFA): 3.656 min; HPLC: Retention time (10 mM NH₄HCO₃)=10.917 min; ¹H NMR (400 MHz, CDCl₃) δ 7.94 (s, 1H), 7.24 (s, 1H), 7.12 (dd, J=10.8, 8.0 Hz, 3H), 6.63 (d, J=7.6 Hz, 2H), 6.59 (dd, J=8.9, 2.2 Hz, 1H), 6.39-6.09 (m, 3H), 4.79 (t, J=7.5 Hz, 1H), 4.39-4.29 (m, 2H), 4.28-4.20 (m, 2H), 3.94 (d, J=5.7 Hz, 2H), 3.76 (s, 1H), 2.96 (d, J=3.7 Hz, 2H), 2.80 (ddd, J=13.5, 7.0, 4.2 Hz, 1H), 2.72 (s, 1H), 2.71-2.65 (m, 1H), 1.53 (s, 1H), 1.21 (d, J=12.3 Hz, 32H), 0.87 (t, J=6.9 Hz, 6H).

### Example 72

### 2-Hexyloctyl (S)-2-(((S)-(((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate

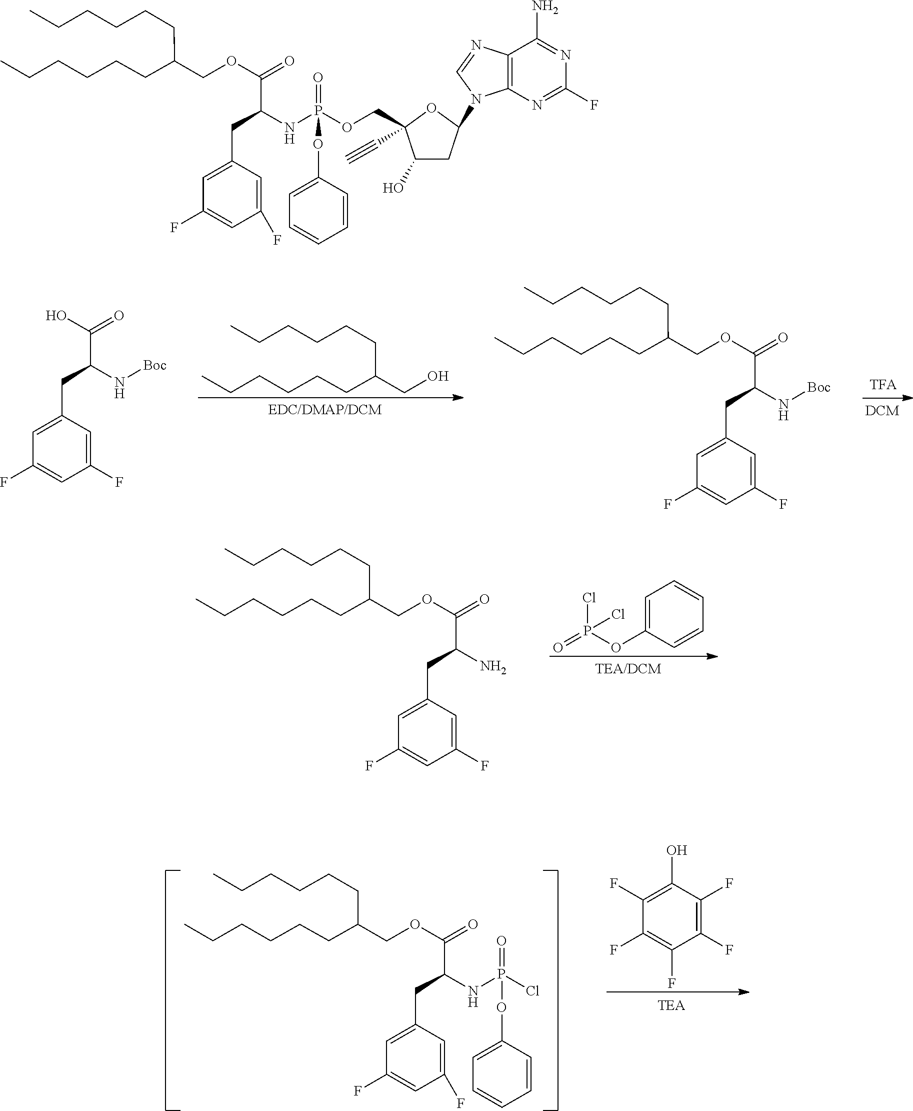

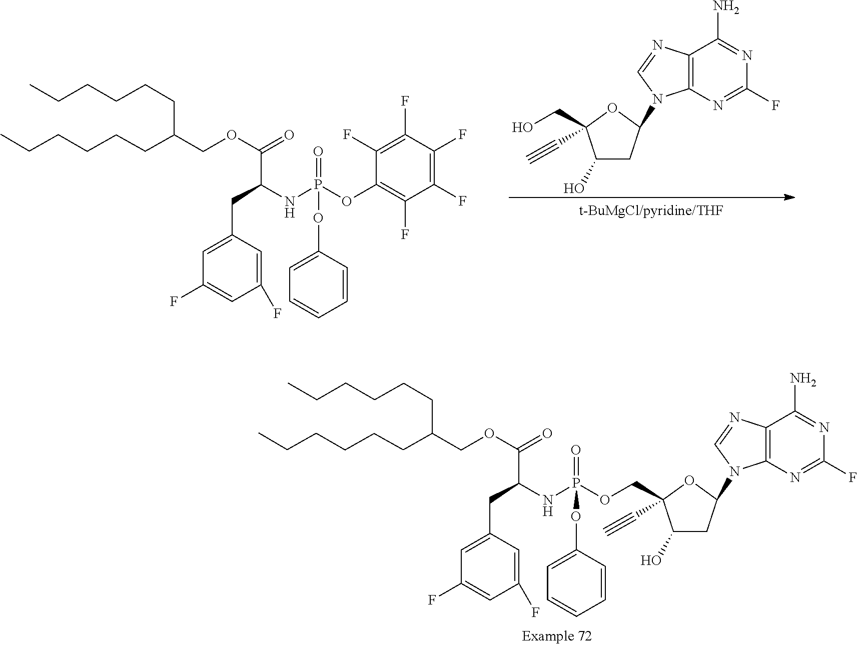 [SMILES: CCCCCCC(CCCCCC)COC(=O)[C@H](Cc1cc(F)cc(F)c1)NP(=O)(Oc1ccccc1)Oc1c(F)c(F)c(F)c(F)c1F ; C#C[C@]1(CO)O[C@@H](n2cnc3c(N)nc(F)nc32)C[C@@H]1O ; C#C[C@]1(CO[P@@](=O)(N[C@@H](Cc2cc(F)cc(F)c2)C(=O)OCC(CCCCCC)CCCCCC)Oc2ccccc2)O[C@@H](n2cnc3c(N)nc(F)nc32)C[C@@H]1O]

### Step 1: 2-Hexyloctyl (S)-2-((tert-butoxycarbonyl)amino)-3-(3,5-difluorophenyl)propanoate

To a stirred mixture of (S)-2-((tert-butoxycarbonyl)amino)-3-(3,5-difluorophenyl)propanoic acid (5 g, 16.60 mmol), DMAP (0.203 g, 1.660 mmol), and 2-hexyloctan-1-ol (4.63 g, 21.60 mmol) was added a solution of EDC (4.14 g, 21.61 mmol) in DCM (20 mL) 0° C. and stirred at 0° C. for 2 h. LCMS showed the presence of desired product. The reaction mixture was diluted with water (50 mL) and the organic phase was washed with brine, dried over Na₂SO₄ and concentrated to give 2-hexyloctyl (S)-2-((tert-butoxycarbonyl)amino)-3-(3,5-difluorophenyl)propanoate (8 g, 15.33 mmol, 92% yield) as an oil. LCMS (M+Na)=520.2; Retention time (0.1% TFA)=3.42 min.

### Step 2: 2-Hexyloctyl (S)-2-amino-3-(3,5-difluorophenyl)propanoate

To a stirred solution of 2-hexyloctyl (S)-2-((tert-butoxycarbonyl)amino)-3-(3,5-difluorophenyl)propanoate (8 g, 16.07 mmol) in DCM (30 mL) was added TFA (12.38 mL, 161 mmol) and the resulting mixture was stirred at 5° C. for 2.5 h. LCMS showed the reaction was completed. The pH of the reaction mixture was adjusted to ˜8 with 1N NaOH, diluted with water (30 mL), vigorously stirred for 20 min, and the organic phase separated and washed with sat. NaCl (50 mL), dried (Na₂SO₄) and concentrated. The crude was purified by silica gel chromatography (120 g, Santai, EtOAc:pet. ether=1:50) to give 2-hexyloctyl (S)-2-amino-3-(3,5-difluorophenyl)propanoate (4.1 g, 7.08 mmol, 44.1 yield) as an oil. LCMS (M+H)=398.4; Retention time (0.1% TFA)=1.92 min.

### Step 3: 2-Hexyloctyl (S)-3-(3,5-difluorophenyl)-2-(((S)-(perfluorophenoxy)(phenoxy)-phosphoryl)amino)propanoate

To a stirred solution of 2-hexyloctyl (S)-2-amino-3-(3,5-difluorophenyl)propanoate (4.1 g, 10.31 mmol) in DCM (50 mL) was added Et₃N (1.509 mL, 10.83 mmol) at −70° C. over 15 min dropwise. To this mixture was added a solution of phenyl phosphorodichloridate (2.154 g, 10.21 mmol) in anhydrous DCM (35 mL) over 1 h. The reaction mixture was stirred at this temperature for additional 30 min and then allowed to warm to 0° C. over 2 h and stirred for 2 h. To this mixture was added a solution of Et₃N (1.581 mL, 11.34 mmol) and 2,3,4,5,6-pentafluorophenol (1.879 g, 10.21 mmol) in DCM (30 mL) over 20 min and stirred at 0° C. for 4 h. LCMS showed the reaction was completed. The white solid (triethylamine hydrochloride) was filtered off and washed with DCM (1×25 mL). The filtrate was concentrated under reduced pressure, the residue was triturated with tert-butyl methyl ether (50 mL), and the triethylamine hydrochloride salt was removed by filtration. The cake was washed with tert-butyl methyl ether (25 mL), and the combined filtrate was concentrated under reduced pressure to give 2.0 g. The crude solid was triturated in pet. ether (10 mL), filtered and washed with pet. ether (5 mL) to give 2-hexyloctyl (S)-3-(3,5-difluorophenyl)-2-(((S)-(perfluorophenoxy)(phenoxy)phosphoryl)amino)propanoate (800 mg, 1.112 mmol, 10.78% yield) as a white solid (>95% de as determined by ³¹PNMR). LCMS (M+H)=720.2, Retention time (0.1% TFA)=3.45 min. ³¹PNMR (CDCl₃, 162 MHz) δ −1.56.

### Step 4: 2-Hexyloctyl (S)-2-(((S)-(((2R,3S,5R)-5-(6-amino-2-fluoro-9H-purin-9-yl)-2-ethynyl-3-hydroxytetrahydrofuran-2-yl)methoxy)(phenoxy)phosphoryl)amino)-3-(3,5-difluorophenyl)propanoate

To a stirred solution of (2R,3S,5R)-5-(6-amino-2-fluoro-9H-purin-9-yl)-2-ethynyl-2-(hydroxymethyl)tetrahydrofuran-3-ol (250 mg, 0.852 mmol) in THF (50 mL) and pyridine (12.50 mL) was added tert-butylmagnesium chloride (1.790 mL, 1.790 mmol) dropwise at −15° C. After 30 min, a solution of 2-hexyloctyl (S)-3-(3,5-difluorophenyl)-2-(((S)-(perfluorophenoxy)(phenoxy)phosphoryl)amino)propanoate (614 mg, 0.852 mmol) in THF (12.5 mL) was added dropwise to the above solution at −15° C. and stirred at −15° C. for 16 h. TLC showed the reaction was completed. The reaction mixture was quenched with sat. NH₄Cl (20 mL) and extracted with EtOAc (50 mL×3). The combined organic layers were washed with brine (50 mL), dried with Na₂SO₄, filtered and concentrated in vacuum. The crude was purified by silica gel chromatography (SanTai, 40 g, DCM:MeOH=20:1) to give 2-hexyloctyl (S)-2-(((S)-(((2R,3S,5R)-5-(6-amino-2-fluoro-9H-purin-9-yl)-2-ethynyl hydroxytetrahydrofuran-2-yl)methoxy)(phenoxy)phosphoryl)amino)-3-(3,5-difluorophenyl)propanoate (216.7 mg, 0.250 mmol, 29.3% yield) as a white solid. ¹H NMR (400 MHz, CDCl₃) δ 8.12 (brs, 1H), 7.30-7.27 (m, 1H), 7.24 (s, 1H), 7.12 (dd, J=13.6, 7.6 Hz, 3H), 6.67-6.62 (m, 2H), 6.59 (dd, J=9.0, 1.9 Hz, 1H), 6.41-6.16 (m, 2H), 4.77 (t, J=7.1 Hz, 1H), 4.39-4.28 (m, 2H), 4.28-4.16 (m, 2H), 3.96 (d, J=5.6 Hz, 2H), 2.98 (d, J=5.4 Hz, 2H), 2.84-2.75 (m, 1H), 2.74-2.66 (m, 2H), 1.31-1.15 (m, 23H), 0.86 (t, J=6.8 Hz, 6H).

### Pharmacokinetics

Protocol for EFdA Rat PK Studies Conducted at Charles River Laboratories, NY.

A total of seven naïve male Wistar Han rats, 200-250 g, were received from Charles River Laboratories, Kingston, N.Y. All animals were received from the supplier equipped with a surgically-implanted jugular vein catheter (JVC) for blood sample collection. Following an acclimation period, the animals were assigned to the study based on acceptable health as determined by a staff veterinarian and catheter patency. Animals were placed into two groups of 3 rats per group. Fasting of the animals before or after dosing was not required. The final study design is presented in the Table 1 below.

TABLE 1

| | | | Dose | Dose | Dose | | |
|---|---|---|---|---|---|---|---|
| | No. of | Test | Level | Conc. | Volume | | |
| Group | Males | Compound | (mg/kg) | (mg/mL) | (mL/kg) | Dose Vehicle | Dose Route |
| 1 | 3 | EFdA | 20 | 60.46 | 0.33 | 0.5% P407/ | Subcutaneous |
| 2 | 3 | | 20 | 60.46 | 0.33 | 0.5% | Intramuscular |
| | | | | | | PEG3350/ | |
| | | | | | | 3.5% Mannitol | |
| | | | | | | in water | |

PEG3350 = Polyethylene glycol 3350.

On Day 1 each animal in Group 1 received a single subcutaneous injection of prepared test article at a target dose level of 20 mg/kg and at a dose volume of 0.33 mL/kg. Each animal in Group 2 received a single intramuscular injection of prepared test article into a thigh muscle at a target dose level of 20 mg/kg and at a dose volume of 0.33 mL/kg. The injection sites were shaved prior to dosing and identified with an indelible marker for daily monitoring during the study. The animals were manually restrained for dosing and were not sedated. The dose suspension was mixed well by inversion before each dose to ensure homogeneity. All dosing was performed as detailed in the study protocol and was completed without incident. Following dosing and at each sample collection time point the animals and injection sites were observed for any clinically relevant abnormalities. In addition, all animals are monitored twice daily by the veterinary staff for clinical abnormalities. No abnormal clinical observations and no injection site reactions were noted during the study period.

Blood samples were collected from the study animals as detailed in the sample collection Table 2 below.

TABLE 2

| Dose Group/ | |
|---|---|
| Collection Information | Whole Blood for PK |
| 1 and 2 | 0.5, 1, 4, 7, 24, 48, 72 hours post-dose |
| | and Day 6, 8, 11, 15, 22, 25, 29 |
| Anticoagulant | NaF/Na₂EDTA |
| Volume/Time point | 250 μL |

Each blood sample for PK (250 μL) was collected from the jugular vein catheter or by venipuncture of a jugular vein. The blood was transferred to a tube containing NaF/Na₂EDTA anticoagulant and inverted several times to mix. The blood samples were maintained on crushed ice before centrifugation at 2200×g for 10 minutes at 5° C. to isolate plasma. The resulting plasma samples were transferred to individual polypropylene tubes in a 96-well plate format and immediately placed on dry ice until storage at nominally −80° C. prior to analysis.

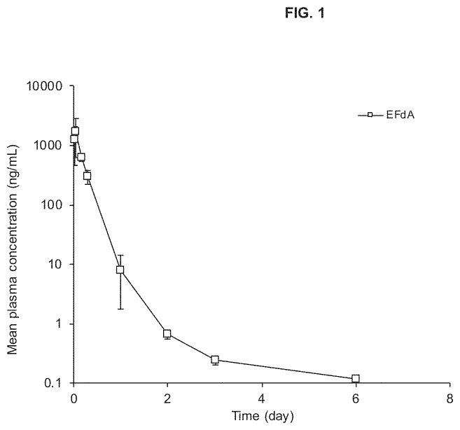

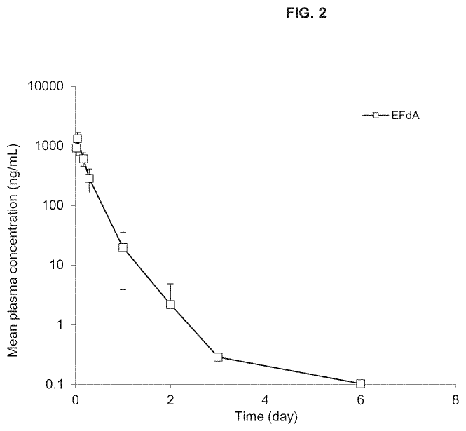

The PK plasma samples were analyzed by the Testing Facility to determine the concentration of EFdA using an LC-MS/MS Research Grade Assay (RGA-1) with sensitivity to 0.1 ng/mL. Following review of sample analysis data through Day 29, further sample analysis was discontinued and the study was terminated. The individual and mean plasma concentration-time data are presented in Tables 3 and 4 and mean concentration-time profiles are shown in FIGS. 1 and 2.

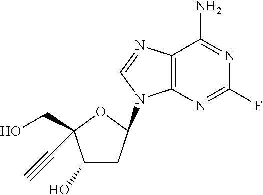 [SMILES: C#C[C@]1(CO)O[C@@H](n2cnc3c(N)nc(F)nc32)C[C@@H]1O]

### (2R,3S,5R)-5-(6-amino-2-fluoro-9H-purin-9-yl)-2-ethynyl-2-(hydroxymethyl)tetrahydrofuran-3-ol

(EFdA)

TABLE 3
Individual and mean plasma concentration-time data of
EFdA from rat SC PK study
Assayed Concentrations (ng/mL)

| Time | Rat#1 | Rat#2 | Rat#3 | Mean |
|---|---|---|---|---|
| 0.5 hr | 1000 | 640 | 2210 | 1283.33 |
| 1 hr | 1160 | 1040 | 3030 | 1430.0 |
| 4 hr | 534 | 676 | 708 | 639.33 |
| 7 hr | 320 | 372 | 216 | 302.67 |
| 24 hr | 6.70 | 14.7 | 2.53 | 7.98 |
| 48 hr | 0.771 | 0.713 | 0.543 | 0.68 |
| 72 hr | 0.251 | 0.270 | 0.201 | 0.241 |
| Day 6 | BQL | BQL | 0.117 | 0.04 |
| Day 8 | BQL | BQL | BQL | BQL |
| Day 11 | BQL | BQL | BQL | BQL |
| Day 15 | BQL | BQL | BQL | BQL |
| Day 22 | BQL | BQL | BQL | BQL |
| Day 25 | 0.221 | BQL | BQL | 0.221 |
| Day 29 | BQL | BQL | BQL | BQL |

BQL—Below Quantitation Limit, <0.100 ng/mL

TABLE 4
Individual and mean plasma concentration-time data of
EFdA from rat IM PK study
Assayed Concentrations (ng/mL)

| Time | Rat#1 | Rat#2 | Rat#3 | Mean |
|---|---|---|---|---|
| 0.5 hr | 929 | 1150 | 684 | 921.0 |
| 1 hr | 957 | 1640 | 1400 | 1332.33 |
| 4 hr | 662 | 436 | 733 | 610.33 |
| 7 hr | 241 | 191 | 426 | 286.0 |
| 24 hr | 24.7 | 1.96 | 32.6 | 19.75 |
| 48 hr | 5.25 | 0.369 | 0.915 | 2.18 |
| 72 hr | 0.301 | 0.316 | 0.241 | 0.29 |
| Day 6 | 0.102 | BQL | 0.103 | 0.10 |
| Day 8 | BQL | BQL | BQL | BQL |
| Day 11 | BQL | BQL | BQL | BQL |
| Day 15 | BQL | BQL | BQL | BQL |
| Day 22 | BQL | BQL | BQL | BQL |
| Day 25 | BQL | BQL | BQL | BQL |
| Day 29 | BQL | BQL | BQL | BQL |

BQL—Below Quantitation Limit, <0.100 ng/mL

General Protocol for Prodrugs Rat PK Studies Conducted at ChemPartner, China

A total of three naïve male Wistar Han rats, 200-250 g, were received from Beijing VR Laboratory Animal Co. LTD. Following an acclimation period, the animals were assigned to the study based on acceptable health as determined by a staff veterinarian and catheter patency. Fasting of the animals before or after dosing was not required.

The final study design is presented in the Table 5 below.

TABLE 5

| No. | Dose | Dose | Dose | | |
|---|---|---|---|---|---|
| of | Level | Conc. | Volume | | Dose |
| Males | (mg/kg) | (mg/mL) | (mL/kg) | Dose Vehicle | Route |
| 3 | 20 | 10 | 2 | 2% Kolliphor | Intra- |
| | (equiva- | | | P407 + 2% | muscular |
| | lent of | | | PEG3350 + | |
| | EFdA | | | 3.5% Mannitol + | |
| | | | | 92.5% Water | |

PEG3350 = Polyethylene glycol 3350.

On Day 1, each animal received a single intramuscular injection of prepared test article at a target dose level of 20 mg/kg equivalent of EFdA and at a dose volume of 2 mL/kg. The injection sites were at gastrocnemius and identified with an indelible marker for daily monitoring during the study. The animals were manually restrained for dosing and were not sedated. The dose suspension was mixed well by inversion before each dose to ensure homogeneity. All dosing was performed as detailed in the study protocol and was completed without incident. Following dosing and at each sample collection time point the animals and injection sites were observed for any clinically relevant abnormalities. In addition, all animals are monitored twice daily by the veterinary staff for clinical abnormalities. No abnormal clinical observations and no injection site reactions were noted during the study period.

Blood samples were collected from the study animals as detailed in the sample collection Table 6 below

TABLE 6

| Collection Information | Whole Blood for PK |
|---|---|
| Collection time | Day 1, 7, 14, 21, 28, 35, 42, 49, 56, 63, 70, |
| | 77 and 84 post dose (the dosing day is Day 0) |
| Anticoagulant | NaF/Na₂EDTA |
| Volume/Time point | 200 μL blood |

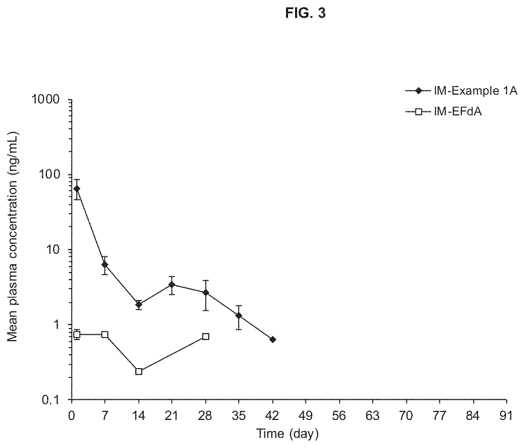

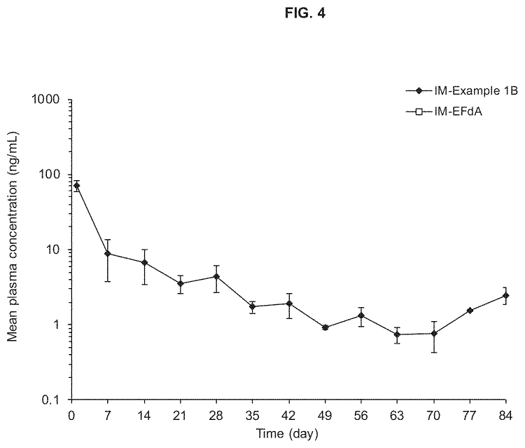

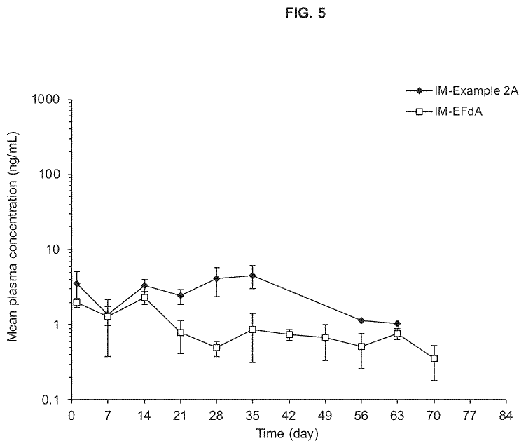

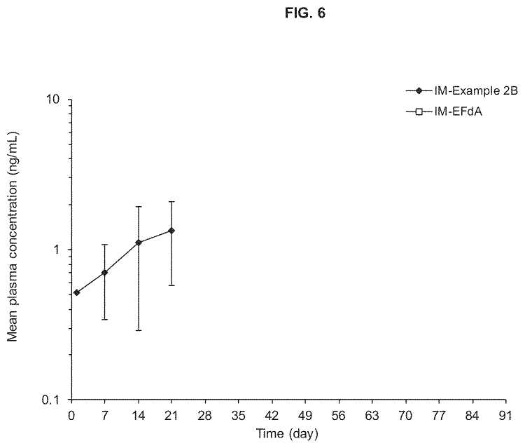

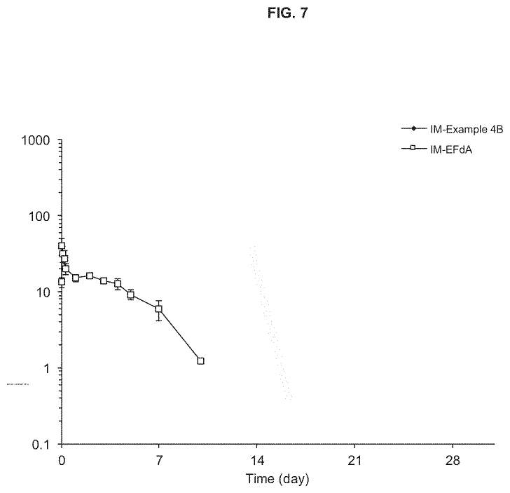

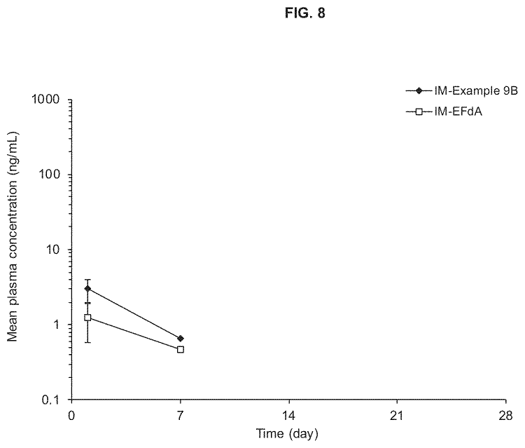

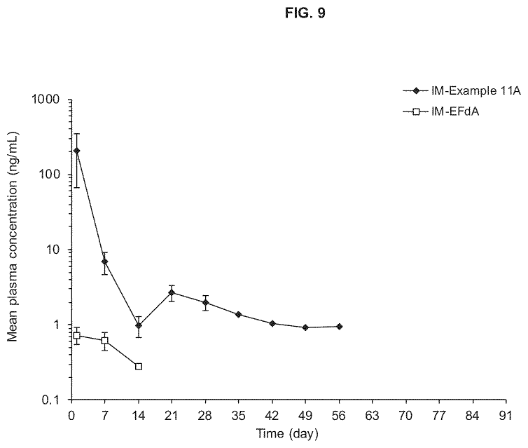

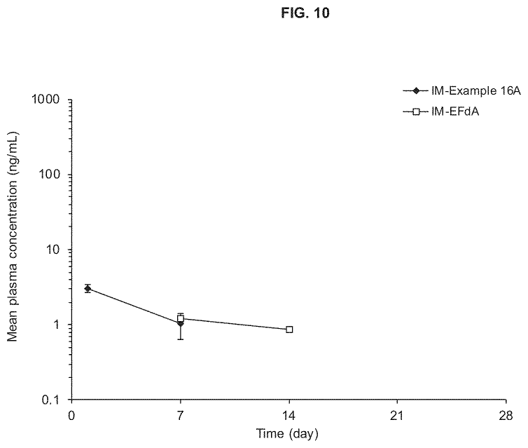

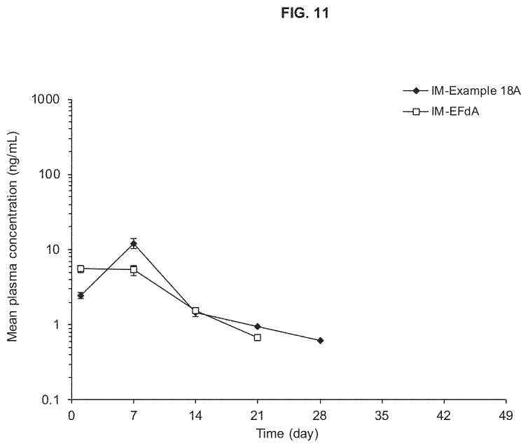

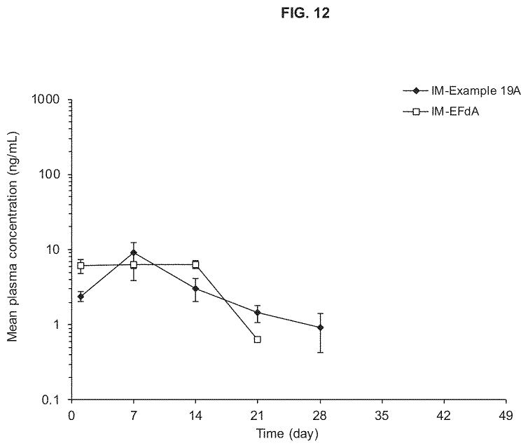

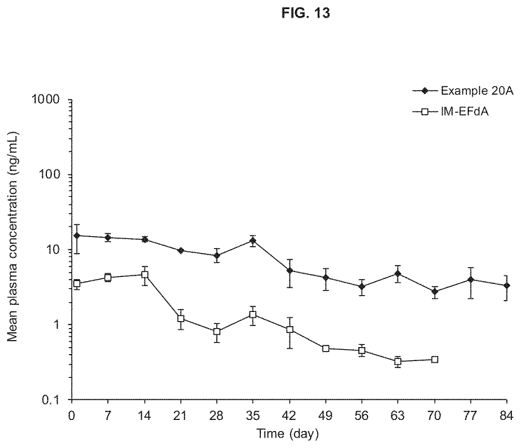

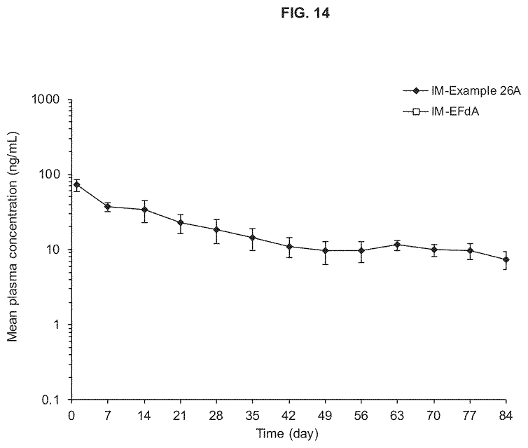

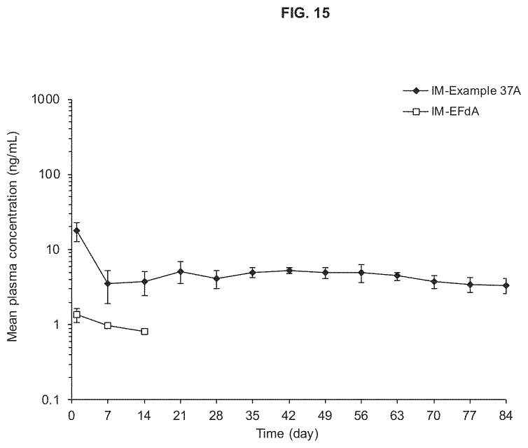

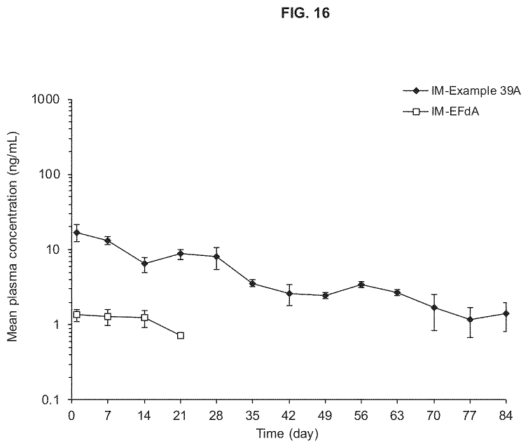

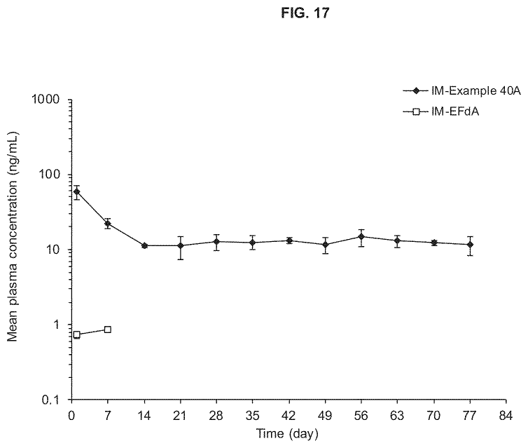

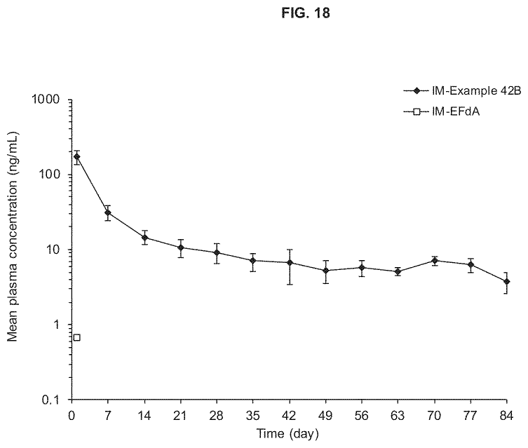

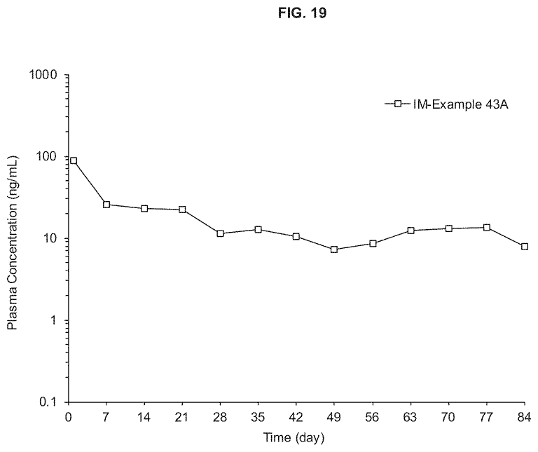

Each blood sample for PK (200 μL) was collected from tail vein. The blood was transferred to a tube containing NaF/Na₂EDTA anticoagulant and inverted several times to mix. Draw 150 μL whole blood from the tube, add 150 uL 100 mM ammonium acetate, pH 4 buffer and mix well. The resulting blood samples were transferred to individual polypropylene tubes and immediately placed on dry ice until storage at nominally −80° C. prior to LC-MS/MS analysis. The mean plasma concentration-time data are presented in Tables 7 to 23 and mean concentration-time profiles are shown in FIGS. 3 to 19.

TABLE 7
Mean plasma concentration-time data of Example 1A (prodrug)
and EFdA (parent) after single intramuscular injection
of Example 1A at 20 mg/kg in male Wistar Han Rats

| | Mean concentration | Mean concentration |
|---|---|---|
| Sampling time | of Example 1A | of EFdA |
| (day) | (ng/mL) | (ng/mL) |
| 1 | 65.3 | 0.747 |
| 7 | 6.38 | 0.747 |
| 14 | 1.86 | 0.242 |
| 21 | 3.42 | BQL |
| 28 | 2.72 | 0.694 |
| 35 | 1.34 | BQL |
| 42 | 0.633 | BQL |
| 49 | BQL | BQL |
| 56 | BQL | BQL |
| 63 | BQL | BQL |
| 70 | BQL | BQL |
| 77 | BQL | BQL |
| 84 | BQL | BQL |
| 91 | BQL | BQL |

BQL = below quantification limit, 0.3-0.5 ng/mL for Example 1A and 0.1-o.3 ng/mL for EFdA

TABLE 8
Mean plasma concentration-time data of Example 1B (prodrug)
and EFdA (parent) after single intramuscular injection
of Example 1B at 62 mg/kg in male Wistar Han Rats

| | Mean concentration | Mean concentration |
|---|---|---|
| Sampling time | of Example 1B | of EFdA |
| (day) | (ng/mL) | (ng/mL) |
| 1 | 70.5 | BQL |
| 7 | 8.75 | BQL |
| 14 | 6.78 | BQL |
| 21 | 3.53 | BQL |
| 28 | 4.36 | BQL |
| 35 | 1.72 | BQL |
| 42 | 1.92 | BQL |
| 49 | 0.912 | BQL |
| 56 | 1.33 | BQL |
| 63 | 0.748 | BQL |
| 70 | 0.767 | BQL |
| 77 | 1.55 | BQL |
| 84 | 2.48 | BQL |

BQL = below quantification limit; 0.3 ng/mL for both

TABLE 9
Mean plasma concentration-time data of Example 2A (prodrug)
and EFdA (parent) after single intramuscular injection
of Example 2A at 56 mg/kg in male Wistar Han Rats

| Sampling | Mean concentration | Mean concentration |
|---|---|---|
| time | of Example 2A | of EFdA |
| (day) | (ng/mL) | (ng/mL) |
| 1 | 3.54 | 1.97 |
| 7 | 1.35 | 1.28 |
| 14 | 3.35 | 2.33 |
| 21 | 2.42 | 0.783 |
| 28 | 4.08 | 0.491 |
| 35 | 4.53 | 0.860 |
| 42 | BQL | 0.743 |
| 49 | BQL | 0.667 |
| 56 | 1.14 | 0.513 |
| 63 | 1.05 | 0.762 |
| 70 | BQL | 0.354 |
| 77 | BQL | BQL |
| 84 | BQL | BQL |

BQL = below quantification limit, o.5 ng/mL for Example 2A and 0.1-0.3 ng/mL for EFdA

TABLE 10
Mean plasma concentration-time data of Example 2B (prodrug)
and EFdA (parent) after single intramuscular injection
of Example 2B at 20 mg/kg in male Wistar Han Rats

| Sampling | Mean concentration | Mean concentration |
|---|---|---|
| time | of Example 2B | of EFdA |
| (day) | (ng/mL) | (ng/mL) |
| 1 | 0.514 | BQL |
| 7 | 0.708 | BQL |
| 14 | 1.11 | BQL |
| 21 | 1.33 | BQL |
| 28 | BQL | BQL |
| 35 | BQL | BQL |

BQL = below quantification limit, 0.2-0.3 ng/mL for both

TABLE 11
Mean plasma concentration-time data of Example 4B (prodrug)
and EFdA (parent) after single intramuscular injection of
at 20 mg/kg equivalent of EFdA in male Wistar Han Rats

| Sampling | Mean concentration | Mean concentration |
|---|---|---|
| time | of Example 4B | of EFdA |
| (day) | (ng/mL) | (ng/mL) |
| 0.0208 | BQL | 13.5 |
| 0.0417 | BQL | 40.2 |
| 0.125 | BQL | 31.6 |
| 0.208 | BQL | 27.3 |
| 0.292 | BQL | 20.1 |
| 1 | BQL | 15.3 |
| 2 | BQL | 16.5 |
| 3 | BQL | 14.1 |
| 4 | BQL | 12.8 |
| 5 | BQL | 9.26 |
| 7 | BQL | 5.97 |
| 10 | | 1.23 |
| 14 | | BQL |
| 17 | | BQL |
| 21 | | BQL |
| 24 | | BQL |
| 28 | | BQL |
| 31 | | BQL |

BQL = below quantification limit, 0.5 ng/mL for Example 4B and 0.5 to 1 ng/mL for EFdA

TABLE 12
Mean plasma concentration-time data of Example 9B (prodrug) and
EFdA (parent) after single intramuscular injection of Example
9B at 20 mg/kg equivalent of EFdA in male Wistar Han Rats

| Sampling | Mean concentration | Mean concentration |
|---|---|---|
| time | of Example 9B | of EFdA |
| (day) | (ng/mL) | (ng/mL) |
| 1 | 3.01 | 1.25 |
| 7 | 0.663 | 0.467 |
| 14 | BQL | BQL |
| 21 | BQL | BQL |
| 28 | BQL | BQL |

BQL = below quantification limit, 0.2-0.3 ng/mL for Example 9B and 0.2 ng/mL for EFdA

TABLE 13
Mean plasma concentration-time data of Example 11A (prodrug) and
EFdA (parent) after single intramuscular injection of Example
11A at 20 mg/kg equivalent of EFdA in male Wistar Han Rats

| Sampling | Mean concentration | Mean concentration |
|---|---|---|
| time | of Example 11A | of EFdA |
| (day) | (ng/mL) | (ng/mL) |
| 1 | 207 | 0.729 |
| 7 | 6.93 | 0.621 |
| 14 | 0.991 | 0.275 |
| 21 | 2.68 | BQL |
| 28 | 2.00 | BQL |
| 35 | 1.35 | BQL |
| 42 | 1.03 | BQL |
| 49 | 0.929 | BQL |
| 56 | 0.950 | BQL |
| 63 | BQL | BQL |
| 70 | BQL | BQL |
| 77 | BQL | BQL |
| 84 | BQL | BQL |
| 91 | BQL | BQL |

BQL = below quantification limit, 0.3-0.5 ng/mL for Example 11A and 0.1-0.3 ng/mL for EFdA

TABLE 14
Mean plasma concentration-time data of Example 16A (prodrug) and
EFdA (parent) after single intramuscular injection of Example
16A at 20 mg/kg equivalent of EFdA in male Wistar Han Rats

| | Mean concentration | Mean concentration |
|---|---|---|
| Sampling time | of Example 16A | of EFdA |
| (day) | (ng/mL) | (ng/mL) |
| 1 | 3.05 | BQL |
| 7 | 1.03 | 1.20 |
| 14 | BQL | 0.856 |
| 21 | BQL | BQL |
| 28 | BQL | BQL |

BQL = below quantification limit, 0.3 ng/mL for both

TABLE 15
Mean plasma concentration-time data of Example 18A (prodrug)
and EFdA (parent) after single intramuscular injection
of Example 18A at 54 mg/kg in male Wistar Han Rats

| Sampling | Mean concentration | Mean concentration |
|---|---|---|
| time | of Example 18A | of EFdA |
| (day) | (ng/mL) | (ng/mL) |
| 1 | 2.47 | 5.55 |
| 7 | 12.0 | 5.35 |
| 14 | 1.44 | 1.55 |
| 21 | 0.947 | 0.674 |
| 28 | 0.614 | BQL |
| 35 | BQL | BQL |
| 42 | BQL | BQL |
| 49 | BQL | BQL |

BQL = below quantification limit, 0.3 ng/mL for both

TABLE 16
Mean plasma concentration-time data of Example 19A (prodrug)
and EFdA (parent) after single intramuscular injection
of Example 19A at 55 mg/kg in male Wistar Han Rats

| Sampling | Mean concentration | Mean concentration |
|---|---|---|
| time | of Example 19A | of EFdA |
| (day) | (ng/mL) | (ng/mL) |
| 1 | 2.37 | 6.09 |
| 7 | 9.08 | 6.27 |
| 14 | 3.06 | 6.40 |
| 21 | 1.45 | 0.635 |
| 28 | 0.909 | BQL |
| 35 | BQL | BQL |
| 42 | BQL | BQL |
| 49 | BQL | BQL |

BQL = below quantification limit, 0.3 ng/mL for both

TABLE 17
Mean plasma concentration-time data of Example 20A (prodrug)
and EFda (parent) after single intramuscular injection
of Example 20A at 57 mg/kg in male Wistar Han Rats

| Sampling | Mean concentration | Mean concentration |
|---|---|---|
| time | of Example 20A | of EFdA |
| (day) | (ng/mL) | (ng/mL) |
| 1 | 15.3 | 3.48 |
| 7 | 14.6 | 4.26 |
| 14 | 13.8 | 4.61 |
| 21 | 9.76 | 1.22 |
| 28 | 8.45 | 0.814 |
| 35 | 13.3 | 1.35 |
| 42 | 5.27 | 0.865 |
| 49 | 4.25 | 0.484 |
| 56 | 3.20 | 0.458 |
| 63 | 4.84 | 0.322 |
| 70 | 2.74 | 0.346 |
| 77 | 4.00 | BQL |
| 84 | 3.32 | BQL |

BQL = below quantification limit, 0.3 ng/mL for Example 20A and 0.1-0.3 ng/mL for EFdA

TABLE 18
Mean plasma concentration-time data of Example 26A
(prodrug) and EFdA (parent) after single intramuscular
injection of at 63 mg/kg in male Wistar Han Rats

| Sampling | Mean concentration | Mean concentration |
|---|---|---|
| time | of Example 26A | of EFdA |
| (day) | (ng/mL) | (ng/mL) |
| 1 | 72.7 | BQL |
| 7 | 37.6 | BQL |
| 14 | 33.8 | BQL |
| 21 | 22.9 | BQL |
| 28 | 18.5 | BQL |
| 35 | 14.4 | BQL |
| 42 | 11.1 | BQL |
| 49 | 9.62 | BQL |
| 56 | 9.80 | BQL |
| 63 | 11.5 | BQL |
| 70 | 9.95 | BQL |
| 77 | 9.69 | BQL |
| 84 | 7.43 | BQL |

BQL = below quantification limit, 0.3 ng/ml for both

TABLE 19
Mean plasma concentration-time data of Example 37A (prodrug)
and EFdA (parent) after single intramuscular injection
of Example 37A at 56 mg/kg in male Wistar Han Rats

| Sampling | Mean concentration | Mean concentration |
|---|---|---|
| time | of Example 37A | of EFdA |
| (day) | (ng/mL) | (ng/mL) |
| 1 | 17.9 | 1.35 |
| 7 | 3.58 | 0.986 |
| 14 | 3.80 | 0.811 |
| 21 | 5.16 | BQL |
| 28 | 4.16 | BQL |
| 35 | 4.95 | BQL |
| 42 | 5.29 | BQL |
| 49 | 4.89 | BQL |
| 56 | 4.94 | BQL |
| 63 | 4.46 | BQL |
| 70 | 3.78 | BQL |
| 77 | 3.46 | BQL |
| 84 | 3.32 | BQL |

BQL = below quantification limit, 0.3 ng/ml for both

TABLE 20
Mean plasma concentration-time data of Example 39A (prodrug)
and EFdA after single intramuscular injection of Example
39A at 58 mg/kg in male Wistar Han Rats

| Sampling | Mean concentration | Mean concentration |
|---|---|---|
| time | of Example 39A | of EFdA |
| (day) | (ng/mL) | (ng/mL) |
| 1 | 17.0 | 1.35 |
| 7 | 13.2 | 1.28 |
| 14 | 6.43 | 1.23 |
| 21 | 8.77 | 0.710 |
| 28 | 7.98 | BQL |
| 35 | 3.56 | BQL |
| 42 | 2.61 | BQL |
| 49 | 2.45 | BQL |
| 56 | 3.42 | BQL |
| 63 | 2.70 | BQL |
| 70 | 1.68 | BQL |
| 77 | 1.18 | BQL |
| 84 | 1.39 | BQL |

BQL = below quantification limit, 0.3 ng/mL for both

TABLE 21
Mean plasma concentration-time data of Example 40A (prodrug)
and EFdA (parent) after single intramuscular injection
of Example 40A at 60 mg/kg in male Wistar Han Rats

| | Mean concentration | Mean concentration |
|---|---|---|
| Sampling time | of Example 40A | of EFdA |
| (day) | (ng/mL) | (ng/mL) |
| 1 | 59.2 | 0.741 |
| 7 | 22.4 | 0.854 |
| 14 | 11.2 | BQL |
| 21 | 11.2 | BQL |
| 28 | 12.7 | BQL |
| 35 | 12.6 | BQL |
| 42 | 13.2 | BQL |
| 49 | 11.7 | BQL |
| 56 | 14.7 | BQL |
| 63 | 13.0 | BQL |
| 70 | 12.2 | BQL |
| 77 | 11.7 | BQL |
| 84 | 10.3 | BQL |

BQL = below quantification limit, 0.3 ng/ml for both

TABLE 22
Mean plasma concentration-time data of Example 42B (prodrug)
and EFdA (parent) after single intramuscular injection
of Example 42B at 64 mg/kg in male Wistar Han Rats

| Sampling | Mean concentration | Mean concentration |
|---|---|---|
| time | of Example 42A | of EFdA |
| (day) | (ng/mL) | (ng/mL) |
| 1 | 170 | 0.683 |
| 7 | 31.4 | BQL |
| 14 | 14.6 | BQL |
| 21 | 10.8 | BQL |
| 28 | 9.21 | BQL |
| 35 | 7.04 | BQL |
| 42 | 6.72 | BQL |
| 49 | 5.30 | BQL |
| 56 | 5.70 | BQL |
| 63 | 5.17 | BQL |
| 70 | 7.07 | BQL |
| 77 | 6.23 | BQL |
| 84 | 3.77 | BQL |

BQL = below quantification limit, 0.3 ng/mL for both

TABLE 23
Mean plasma concentration-time data of Example 43A (prodrug)
and EFdA (parent) after single intramuscular injection
of Example 43A at 60 mg/kg in male Wistar Han Rats

| Sampling | Mean concentration | Mean concentration |
|---|---|---|
| time | of Example 43A | of EFdA |
| (day) | (ng/mL) | (ng/mL) |
| 1 | 89.3 | BQL |
| 7 | 25.4 | BQL |
| 14 | 22.7 | BQL |
| 21 | 22.1 | BQL |
| 28 | 11.3 | BQL |
| 35 | 12.7 | BQL |
| 42 | 10.4 | BQL |
| 49 | 7.22 | BQL |
| 56 | 8.67 | BQL |
| 63 | 12.5 | BQL |
| 70 | 13.1 | BQL |
| 77 | 13.5 | BQL |
| 84 | 7.94 | BQL |

BQL = below quantification limit, 0.3 ng/mL for both

As shown above pharmacokinetic results, in rat IM and SC administration of 20 mg/kg EFdA (parent) showed a detectable duration of exposure of EFdA for less than 8-days, likely reflecting rapid absorption of the compound as a consequence of its high solubility and high permeability. Therefore, an approach involving design of novel lipophilic prodrugs of EFdA as a means to modulate the physicochemical properties of the EFdA as a strategy to improve its suitability for use as a long-acting injectable antiviral agent was pursued. The rat IM data demonstrate that sustained exposure of EFdA and/or prodrug for extended period was achieved with several lipophilic prodrugs suggests potential utility of lipophilic prodrugs in HIV therapy as a long-acting IM or SC administrative prodrugs of EFdA.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present disclosure.

1. A compound of formula (I): 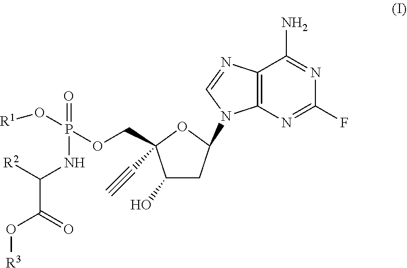
wherein:
R¹ is selected from the group consisting of (C₁-C₂₅) alkyl, (C₁-C₂₅) alkylaryl, (C₁-C₂₀) alkyl-CO₂R⁴ and aryl;
R² is selected from the group consisting of (C₁-C₁₀) alkyl; (C₁-C₁₀) alkylaryl and aryl;
R³ is selected from the group consisting of (C₁-C₂₅) alkyl, (C₁-C₂₅) alkylaryl and aryl; and
R⁴ is selected from the group consisting of (C₁-C₂₅) alkyl, (C₁-C₂₅) alkylaryl and aryl; and
wherein each of R¹, R², R³, and R⁴ may be independently and optionally substituted by one or more (C₁-C₁₄) alkyl, Cl, F, oxo, or (C₁-C₆) alkoxy;
or a pharmaceutically acceptable salt thereof.

2. The compound according to claim 1, or a pharmaceutically acceptable salt thereof, wherein R¹ is (C₆-C₁₄)aryl.

3. The compound according to claim 2, or a pharmaceutically acceptable salt thereof, wherein R¹ is C₆ aryl.

4. The compound according to claim 1, or a pharmaceutically acceptable salt thereof, wherein R¹ is (C₁-C₂₀) alkyl-CO₂R₄.

5. The compound according to claim 4, or a pharmaceutically acceptable salt thereof, wherein R¹ is selected from (C₁-C₂₀) alkyl-CO₂R⁴, wherein R⁴ is (C₁-C₂₅) alkyl.

6. The compound according to claim 1, or a pharmaceutically acceptable salt thereof, wherein R² is (C₁-C₁₀)alkyl(C₆-C₁₄)aryl.

7. The compound according to claim 1, or a pharmaceutically acceptable salt thereof, wherein R² is C₁ alkyl C₆aryl.

8. The compound according to claim 1, or a pharmaceutically acceptable salt thereof, wherein R³ is (C₁-C₂₅)alkyl.

9. The compound according to claim 1, or a pharmaceutically acceptable salt thereof, wherein R¹ is C₆ aryl, R² is C₁ alkyl or C₁ alkyl C₆aryl and R³ is (C₁-C₂₅)alkyl.

10. The compound according to claim 1, or a pharmaceutically acceptable salt thereof, wherein R¹ is (C₁-C₂₅)alkyl-CO₂-R⁴, R² is selected from C₁ alkyl or C₁ alkyl C₆ aryl, and R³ is (C₁-C₂₅)alky

11. The compound according to claim 1, or a pharmaceutically acceptable salt thereof, wherein R² is (C₁-C₁₀)alkyl.

12. The compound according to claim 1, or a pharmaceutically acceptable salt thereof, wherein R² is (C₁-C₅) alkyl.

13. A compound selected from the group consisting of:

| Parent Structure | Chemical Name |
|---|---|
| 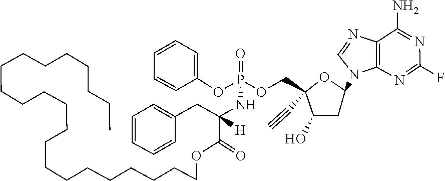 [SMILES: C#C[C@]1(CO[P@@](=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1O] | Docosyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)-L- phenylalaninate |
| 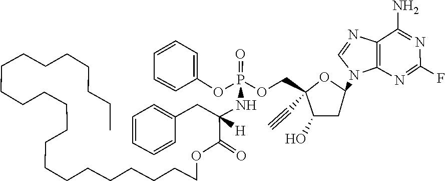 [SMILES: C#C[C@]1(CO[P@](=O)(N[C@@H](Cc2ccccc2)C(=O)OCCCCCCCCCCCCCCCCCCCCCC)Oc2ccccc2)O[C@@H](n2cnc3c(N)nc(F)nc32)C[C@@H]1O] | Docosyl ((R)-(((2R,3S,5R)-5-(6-amino-2- fluoro-9H-purin-9-yl)-2-ethynyl-3- hydroxytetrahydrofuran-2- yl)methoxy)(phenoxy)phosphoryl)-L- phenylalaninate |
| 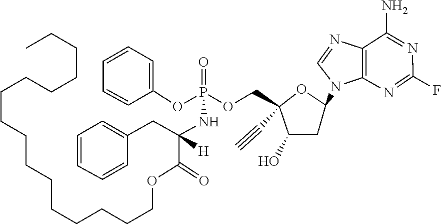 [SMILES: C#C[C@]1(CO[P@@](=O)(N[C@@H](Cc2ccccc2)C(=O)OCCCCCCCCCCCCCCCC)Oc2ccccc2)O[C@@H](n2cnc3c(N)nc(F)nc32)C[C@@H]1O] | Hexadecyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)-L- phenylalaninate |
| 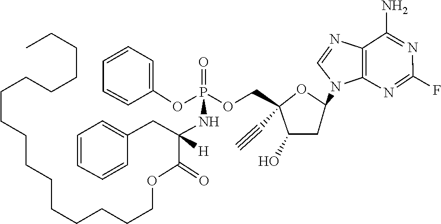 [SMILES: C#C[C@]1(CO[P@](=O)(N[C@@H](Cc2ccccc2)C(=O)OCCCCCCCCCCCCCCCC)Oc2ccccc2)O[C@@H](n2cnc3c(N)nc(F)nc32)C[C@@H]1O] | Hexadecyl ((R)-(((2R,3S,5R)-5-(6- amino-2-fluoro-9H-purin-9-yl)-2- ethynyl-3-hydroxytetrahydrofuran-2-yl) methoxy)(phenoxy)phosphoryl)-L- phenylalaninate |
| 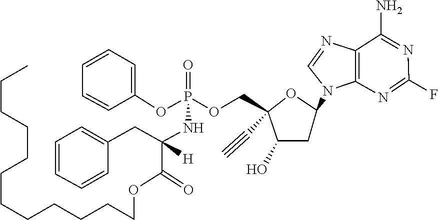 [SMILES: C#C[C@]1(CO[P@@](=O)(N[C@@H](Cc2ccccc2)C(=O)OCCCCCCCCCCCC)Oc2ccccc2)O[C@@H](n2cnc3c(N)nc(F)nc32)C[C@@H]1O] | Dodecyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)-L- phenylalaninate |
| 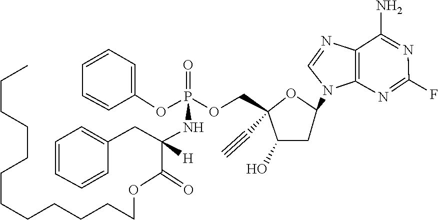 [SMILES: C#C[C@]1(CO[P@](=O)(N[C@@H](Cc2ccccc2)C(=O)OCCCCCCCCCCCC)Oc2ccccc2)O[C@@H](n2cnc3c(N)nc(F)nc32)C[C@@H]1O] | Dodecyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)-L- phenylalaninate |
| 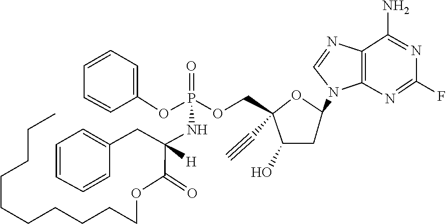 [SMILES: C#C[C@]1(CO[P@@](=O)(N[C@@H](Cc2ccccc2)C(=O)OCCCCCCCCCC)Oc2ccccc2)O[C@@H](n2cnc3c(N)nc(F)nc32)C[C@@H]1O] | Decyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)-L- phenylalaninate |
| 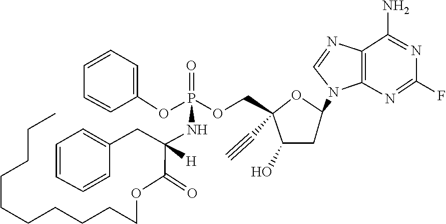 [SMILES: C#C[C@]1(CO[P@](=O)(N[C@@H](Cc2ccccc2)C(=O)OCCCCCCCCCC)Oc2ccccc2)O[C@@H](n2cnc3c(N)nc(F)nc32)C[C@@H]1O] | Decyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphory)-L- phenylalaninate |
| 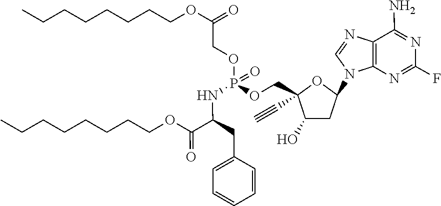 [SMILES: C#C[C@]1(CO[P@@](=O)(N[C@@H](Cc2ccccc2)C(=O)OCCCCCCCC)OCC(=O)OCCCCCCCC)O[C@@H](n2cnc3c(N)nc(F)nc32)C[C@@H]1O] | Octyl ((S)-(((2R,3S,5R)-5-(6-amino-2- fluoro-9H-purin-9-yl)-2-ethynyl-3- hydroxytetrahydrofuran-2-yl) methoxy)(2-(octyloxy)-2-oxoethoxy) phosphoryl)-L-phenylalaninate |
| 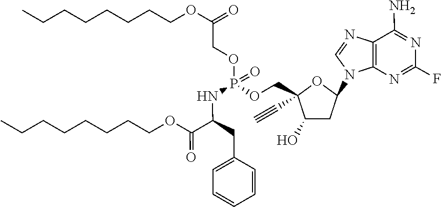 [SMILES: C#C[C@]1(CO[P@](=O)(N[C@@H](Cc2ccccc2)C(=O)OCCCCCCCC)OCC(=O)OCCCCCCCC)O[C@@H](n2cnc3c(N)nc(F)nc32)C[C@@H]1O] | Octyl ((R)-(((2R,3S,5R)-5-(6-amino-2- fluoro-9H-purin-9-yl)-2-ethynyl-3- hydroxytetrahydrofuran-2-yl) methoxy)(2-(octyloxy)-2- oxoethoxy)phosphoryl)-L- phenylalaninate |
| 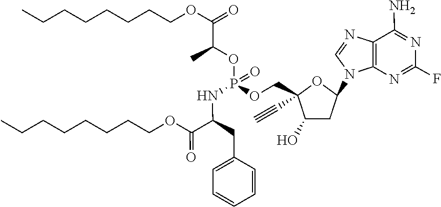 [SMILES: C#C[C@]1(CO[P@@](=O)(N[C@@H](Cc2ccccc2)C(=O)OCCCCCCCC)O[C@@H](C)C(=O)OCCCCCCCC)O[C@@H](n2cnc3c(N)nc(F)nc32)C[C@@H]1O] | Octyl ((S)-(((2R,3S,5R)-5-(6-amino-2- fluoro-9H-purin-9-yl)-2-ethynyl-3- hydroxytetrahydrofuran-2-yl)methoxy) (((S)-1-(octyloxy)-1-oxopropan-2-yl) oxy)phosphoryl)-L-phenylalaninate |
| 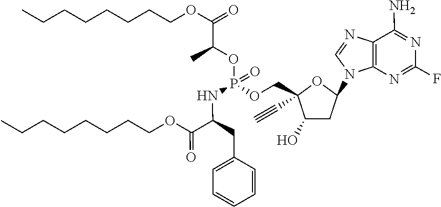 [SMILES: C#C[C@]1(CO[P@](=O)(N[C@@H](Cc2ccccc2)C(=O)OCCCCCCCC)O[C@@H](C)C(=O)OCCCCCCCC)O[C@@H](n2cnc3c(N)nc(F)nc32)C[C@@H]1O] | Octyl ((R)-(((2R,3S,5R)-5-(6-amino- 2-fluoro-9H-purin-9-yl)-2-ethynyl-3- hydroxytetrahydrofuran-2-yl)methoxy) (((S)-1-(octyloxy)-1-oxopropan-2-yl) oxy)phosphoryl)-L-phenylalaninate |
| 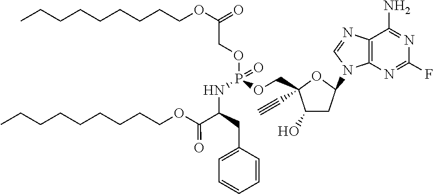 [SMILES: C#C[C@]1(CO[P@@](=O)(N[C@@H](Cc2ccccc2)C(=O)OCCCCCCCCC)OCC(=O)OCCCCCCCCC)O[C@@H](n2cnc3c(N)nc(F)nc32)C[C@@H]1O] | Nonyl ((S)-(((2R,3S,5R)-5-(6-amino- 2-fluoro-9H-purin-9-yl)-2-ethynyl-3- hydroxytetrahydrofuran-2-yl)methoxy) (2-(nonyloxy)-2-oxoethoxy)phosphoryl)- L-phenylalaninate |
| 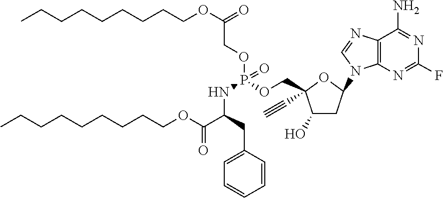 [SMILES: C#C[C@]1(CO[P@](=O)(N[C@@H](Cc2ccccc2)C(=O)OCCCCCCCCC)OCC(=O)OCCCCCCCCC)O[C@@H](n2cnc3c(N)nc(F)nc32)C[C@@H]1O] | Nonyl ((R)-(((2R,3S,5R)-5-(6-amino- 2-fluoro-9H-purin-9-yl)-2-ethynyl-3- hydroxytetrahydrofuran-2-yl)methoxy) (2-(nonyloxy)-2-oxoethoxy)phosphoryl)- L-phenylalaninate |
| 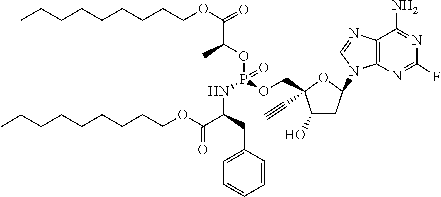 | Nonyl ((S)-(((2R,3S,5R)-5-(6-amino- 2-fluoro-9H-purin-9-yl)-2-ethynyl-3- hydroxytetrahydrofuran-2-yl)methoxy) (((S)-1-(nonyloxy)-1-oxopropan-2- yl)oxy)phosphoryl)-L-phenylalaninate |
| 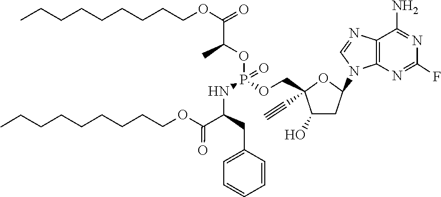 [SMILES: C#C[C@]1(CO[P@](=O)(N[C@@H](Cc2ccccc2)C(=O)OCCCCCCCCC)O[C@@H](C)C(=O)OCCCCCCCCC)O[C@@H](n2cnc3c(N)nc(F)nc32)C[C@@H]1O] | Nonyl ((R)-(((2R,3S,5R)-5-(6-amino- 2-fluoro-9H-purin-9-yl)-2-ethynyl-3- hydroxytetrahydrofuran-2-yl)methoxy) (((S)-1-(nonyloxy)-1-oxopropan-2-yl) oxy)phosphoryl)-L-phenylalaninate |
| 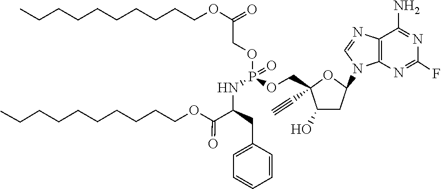 [SMILES: C#C[C@]1(CO[P@@](=O)(N[C@@H](Cc2ccccc2)C(=O)OCCCCCCCCCC)OCC(=O)OCCCCCCCCCC)O[C@@H](n2cnc3c(N)nc(F)nc32)C[C@@H]1O] | Decyl ((S)-(((2R,3S,5R)-5-(6-amino- 2-fluoro-9H-purin-9-yl)-2-ethynyl-3- hydroxytetrahydrofuran-2-yl)methoxy) (2-(decyloxy)-2-oxoethoxy)phosphoryl)- L-phenylalaninate |
| 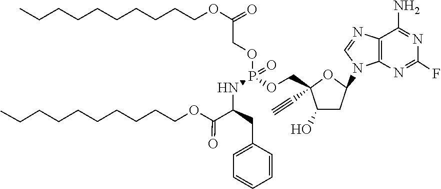 [SMILES: C#C[C@]1(CO[P@](=O)(N[C@@H](Cc2ccccc2)C(=O)OCCCCCCCCCC)OCC(=O)OCCCCCCCCCC)O[C@@H](n2cnc3c(N)nc(F)nc32)C[C@@H]1O] | Decyl ((R)-(((2R,3S,5R)-5-(6-amino- 2-fluoro-9H-purin-9-yl)-2-ethynyl-3- hydroxytetrahydrofuran-2-yl)methoxy) (2-(decyloxy)-2-oxoethoxy)phosphoryl)- L-phenylalaninate |
| 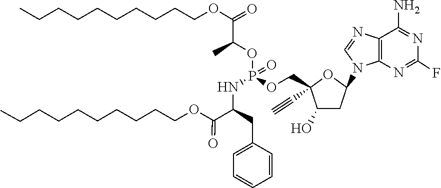 [SMILES: C#C[C@]1(CO[P@@](=O)(N[C@@H](Cc2ccccc2)C(=O)OCCCCCCCCCC)O[C@@H](C)C(=O)OCCCCCCCCCC)O[C@@H](n2cnc3c(N)nc(F)nc32)C[C@@H]1O] | Decyl ((S)-(((2R,3S,5R)-5-(6-amino- 2-fluoro-9H-purin-9-yl)-2-ethynyl-3- hydroxytetrahydrofuran-2-yl)methoxy) (((S)-1-(decyloxy)-1-oxopropan-2-yl) oxy)phosphoryl)-L-phenylalaninate |
| 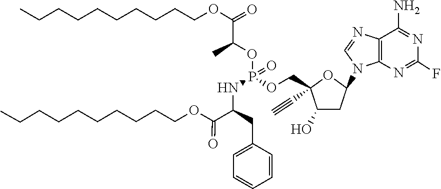 [SMILES: C#C[C@]1(CO[P@](=O)(N[C@@H](Cc2ccccc2)C(=O)OCCCCCCCCCC)O[C@@H](C)C(=O)OCCCCCCCCCC)O[C@@H](n2cnc3c(N)nc(F)nc32)C[C@@H]1O] | Decyl ((R)-(((2R,3S,5R)-5-(6-amino- 2-fluoro-9H-purin-9-yl)-2-ethynyl-3- hydroxytetrahydrofuran-2-yl)methoxy) (((S)-1-(decyloxy)-1-oxopropan-2-yl) oxy)phosphoryl)-L-phenylalaninate |
| 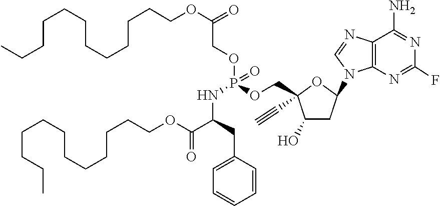 [SMILES: C#C[C@]1(CO[P@@](=O)(N[C@@H](Cc2ccccc2)C(=O)OCCCCCCCCCCCC)OCC(=O)OCCCCCCCCCCCC)O[C@@H](n2cnc3c(N)nc(F)nc32)C[C@@H]1O] | Dodecyl ((S)-(((2R,3S,5R)-5-(6-amino- 2-fluoro-9H-purin-9-yl)-2-ethynyl-3- hydroxytetrahydrofuran-2-yl)methoxy) (2-(dodecyloxy)-2-oxoethoxy)phosphoryl)- L-phenylalaninate |
| 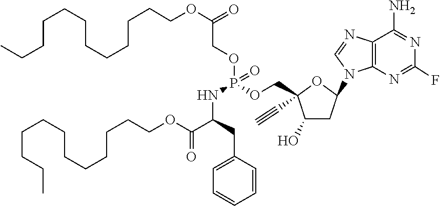 [SMILES: C#C[C@]1(CO[P@](=O)(N[C@@H](Cc2ccccc2)C(=O)OCCCCCCCCCCCC)OCC(=O)OCCCCCCCCCCCC)O[C@@H](n2cnc3c(N)nc(F)nc32)C[C@@H]1O] | Dodecyl ((R)-(((2R,3S,5R)-5-(6-amino- 2-fluoro-9H-purin-9-yl)-2-ethynyl-3- hydroxytetrahydrofuran-2-yl)methoxy) (2-(dodecyloxy)-2-oxoethoxy)phosphoryl)- L-phenylalaninate |
| 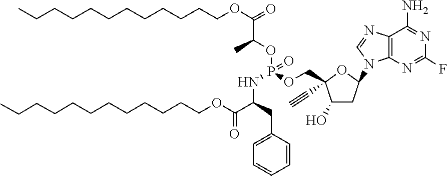 [SMILES: C#C[C@]1(CO[P@@](=O)(N[C@@H](Cc2ccccc2)C(=O)OCCCCCCCCCCCC)O[C@@H](C)C(=O)OCCCCCCCCCCCC)O[C@@H](n2cnc3c(N)nc(F)nc32)C[C@@H]1O] | Dodecyl ((S)-(((2R,3S,5R)-5-(6-amino- 2-fluoro-9H-purin-9-yl)-2-ethynyl-3- hydroxytetrahydrofuran-2-yl)methoxy) (((S)-1-(dodecyloxy)-1-oxopropan-2-yl) oxy)phosphoryl)-L-phenylalaninate |
| 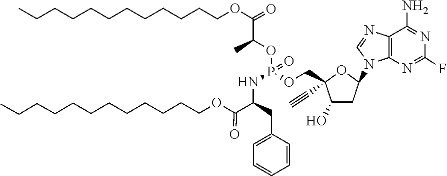 [SMILES: C#C[C@]1(CO[P@](=O)(N[C@@H](Cc2ccccc2)C(=O)OCCCCCCCCCCCC)O[C@@H](C)C(=O)OCCCCCCCCCCCC)O[C@@H](n2cnc3c(N)nc(F)nc32)C[C@@H]1O] | Dodecyl ((R)-(((2R,3S,5R)-5-(6-amino- 2-fluoro-9H-purin-9-yl)-2-ethynyl-3- hydroxytetrahydrofuran-2-yl)methoxy) (((S)-1-(dodecyloxy)-1-oxopropan-2-yl) oxy)phosphoryl)-L-phenylalaninate |
| 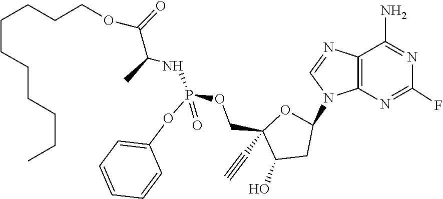 [SMILES: C#C[C@]1(CO[P@](=O)(N[C@@H](C)C(=O)OCCCCCCCCCC)Oc2ccccc2)O[C@@H](n2cnc3c(N)nc(F)nc32)C[C@@H]1O] | Decyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 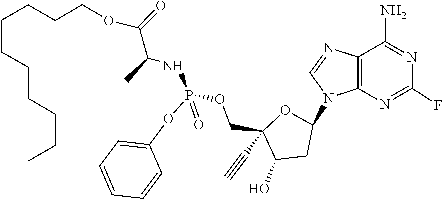 [SMILES: C#C[C@]1(CO[P@@](=O)(N[C@@H](C)C(=O)OCCCCCCCCCC)Oc2ccccc2)O[C@@H](n2cnc3c(N)nc(F)nc32)C[C@@H]1O] | Decyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 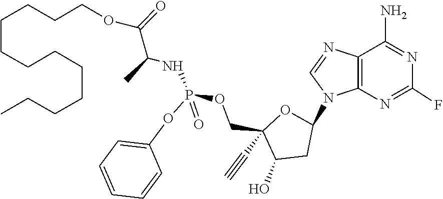 [SMILES: C#C[C@]1(CO[P@](=O)(N[C@@H](C)C(=O)OCCCCCCCCCCCC)Oc2ccccc2)O[C@@H](n2cnc3c(N)nc(F)nc32)C[C@@H]1O] | Dodecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 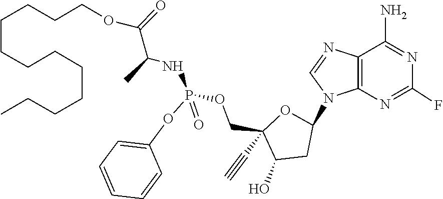 [SMILES: C#C[C@]1(CO[P@@](=O)(N[C@@H](C)C(=O)OCCCCCCCCCCCC)Oc2ccccc2)O[C@@H](n2cnc3c(N)nc(F)nc32)C[C@@H]1O] | Dodecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 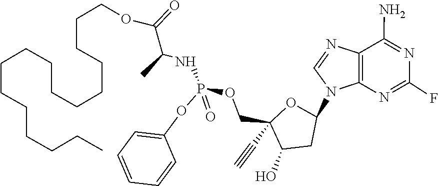 [SMILES: C#C[C@]1(CO[P@](=O)(N[C@@H](C)C(=O)OCCCCCCCCCCCCCCCC)Oc2ccccc2)O[C@@H](n2cnc3c(N)nc(F)nc32)C[C@@H]1O] | Hexadecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 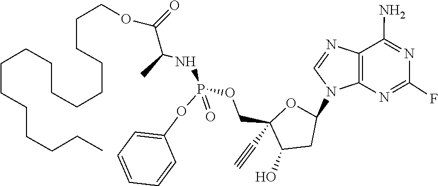 [SMILES: C#C[C@]1(CO[P@@](=O)(N[C@@H](C)C(=O)OCCCCCCCCCCCCCCCC)Oc2ccccc2)O[C@@H](n2cnc3c(N)nc(F)nc32)C[C@@H]1O] | Hexadecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 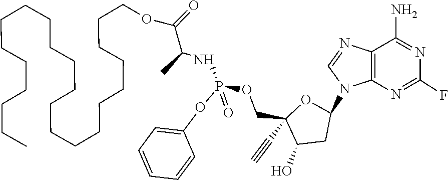 [SMILES: C#C[C@]1(CO[P@](=O)(N[C@@H](C)C(=O)OCCCCCCCCCCCCCCCCCCCCCC)Oc2ccccc2)O[C@@H](n2cnc3c(N)nc(F)nc32)C[C@@H]1O] | Docosyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 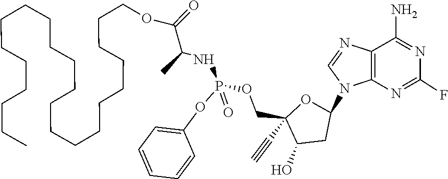 [SMILES: C#C[C@]1(CO[P@@](=O)(N[C@@H](C)C(=O)OCCCCCCCCCCCCCCCCCCCCCC)Oc2ccccc2)O[C@@H](n2cnc3c(N)nc(F)nc32)C[C@@H]1O] | Docosyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 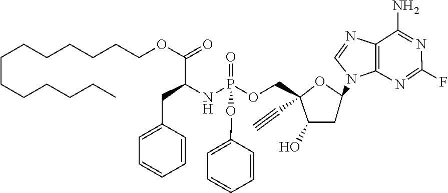 [SMILES: C#C[C@]1(CO[P@](=O)(N[C@@H](Cc2ccccc2)C(=O)OCCCCCCCCCCCCC)Oc2ccccc2)O[C@@H](n2cnc3c(N)nc(F)nc32)C[C@@H]1O] | Tridecyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 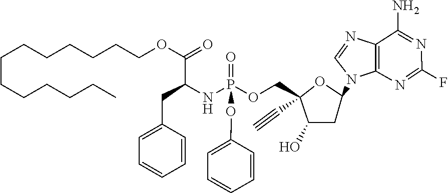 [SMILES: C#C[C@]1(CO[P@@](=O)(N[C@@H](Cc2ccccc2)C(=O)OCCCCCCCCCCCCC)Oc2ccccc2)O[C@@H](n2cnc3c(N)nc(F)nc32)C[C@@H]1O] | Tridecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 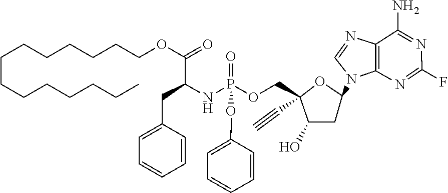 [SMILES: C#C[C@]1(CO[P@](=O)(N[C@@H](Cc2ccccc2)C(=O)OCCCCCCCCCCCCCC)Oc2ccccc2)O[C@@H](n2cnc3c(N)nc(F)nc32)C[C@@H]1O] | Tetradecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 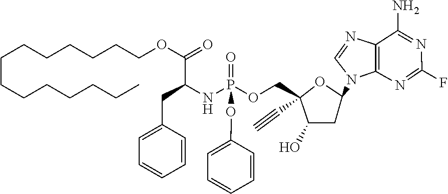 [SMILES: C#C[C@]1(CO[P@@](=O)(N[C@@H](Cc2ccccc2)C(=O)OCCCCCCCCCCCCCC)Oc2ccccc2)O[C@@H](n2cnc3c(N)nc(F)nc32)C[C@@H]1O] | Tetradecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 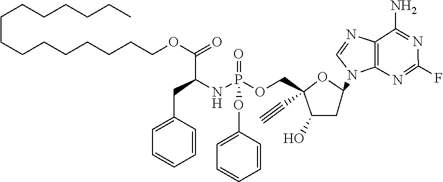 [SMILES: C#C[C@]1(CO[P@](=O)(N[C@@H](Cc2ccccc2)C(=O)OCCCCCCCCCCCCCCC)Oc2ccccc2)O[C@@H](n2cnc3c(N)nc(F)nc32)C[C@@H]1O] | Pentadecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 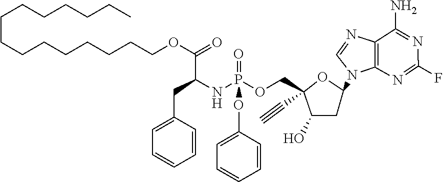 [SMILES: C#C[C@]1(CO[P@@](=O)(N[C@@H](Cc2ccccc2)C(=O)OCCCCCCCCCCCCCCC)Oc2ccccc2)O[C@@H](n2cnc3c(N)nc(F)nc32)C[C@@H]1O] | Pentadecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 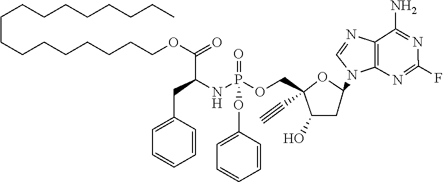 [SMILES: C#C[C@]1(CO[P@](=O)(N[C@@H](Cc2ccccc2)C(=O)OCCCCCCCCCCCCCCCCC)Oc2ccccc2)O[C@@H](n2cnc3c(N)nc(F)nc32)C[C@@H]1O] | Heptadecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 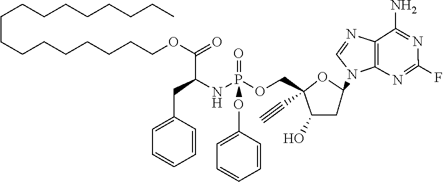 [SMILES: C#C[C@]1(CO[P@@](=O)(N[C@@H](Cc2ccccc2)C(=O)OCCCCCCCCCCCCCCCCC)Oc2ccccc2)O[C@@H](n2cnc3c(N)nc(F)nc32)C[C@@H]1O] | Heptadecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 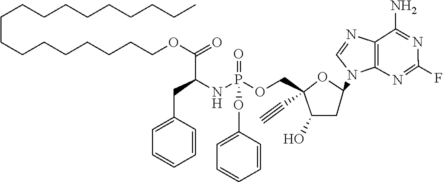 [SMILES: C#C[C@]1(CO[P@](=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1O] | Octadecyl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 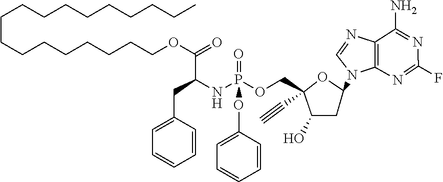 [SMILES: C#C[C@]1(CO[P@@](=O)(N[C@@H](Cc2ccccc2)C(=O)OCCCCCCCCCCCCCCCCCC)Oc2ccccc2)O[C@@H](n2cnc3c(N)nc(F)nc32)C[C@@H]1O] | Octadecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 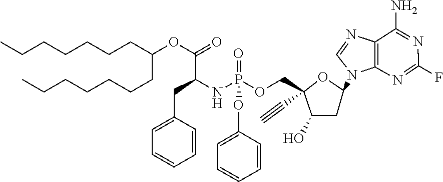 [SMILES: C#C[C@]1(CO[P@](=O)(N[C@@H](Cc2ccccc2)C(=O)OC(CCCCCCC)CCCCCCC)Oc2ccccc2)O[C@@H](n2cnc3c(N)nc(F)nc32)C[C@@H]1O] | Pentadecan-8-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 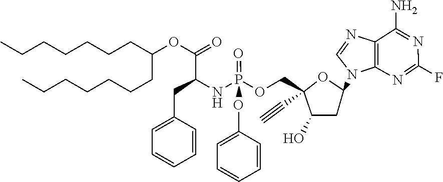 [SMILES: C#C[C@]1(CO[P@@](=O)(N[C@@H](Cc2ccccc2)C(=O)OC(CCCCCCC)CCCCCCC)Oc2ccccc2)O[C@@H](n2cnc3c(N)nc(F)nc32)C[C@@H]1O] | Pentadecan-8-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 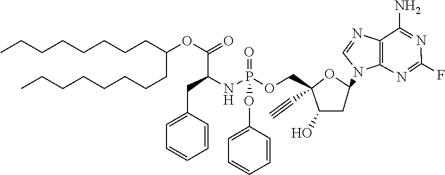 [SMILES: C#C[C@]1(CO[P@](=O)(N[C@@H](Cc2ccccc2)C(=O)OC(CCCCCCCC)CCCCCCCC)Oc2ccccc2)O[C@@H](n2cnc3c(N)nc(F)nc32)C[C@@H]1O] | Heptadecan-9-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 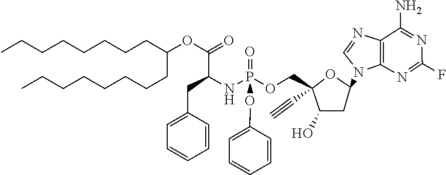 [SMILES: C#C[C@]1(CO[P@@](=O)(N[C@@H](Cc2ccccc2)C(=O)OC(CCCCCCCC)CCCCCCCC)Oc2ccccc2)O[C@@H](n2cnc3c(N)nc(F)nc32)C[C@@H]1O] | Heptadecan-9-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 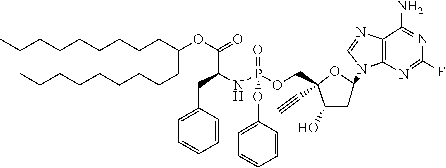 [SMILES: C#C[C@]1(CO[P@](=O)(N[C@@H](Cc2ccccc2)C(=O)OC(CCCCCCCCC)CCCCCCCCC)Oc2ccccc2)O[C@@H](n2cnc3c(N)nc(F)nc32)C[C@@H]1O] | Nonadecan-10-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 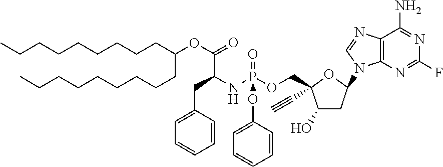 [SMILES: C#C[C@]1(CO[P@@](=O)(N[C@@H](Cc2ccccc2)C(=O)OC(CCCCCCCCC)CCCCCCCCC)Oc2ccccc2)O[C@@H](n2cnc3c(N)nc(F)nc32)C[C@@H]1O] | Nonadecan-10-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 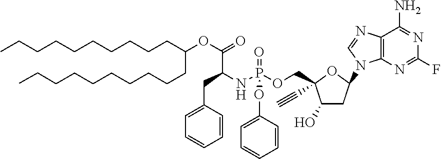 [SMILES: C#C[C@]1(CO[P@](=O)(N[C@@H](Cc2ccccc2)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((R)-(((2R,3S,5R)-5- (6-amino-2-fluoro-9H-purin-9-yl)-2-ethynyl- 3-hydroxytetrahydrofuran-2-yl) methoxy)(phenoxy)phosphoryl)- L-phenylalaninate |
| 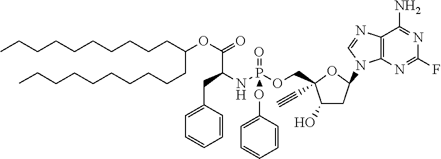 [SMILES: C#C[C@]1(CO[P@@](=O)(N[C@@H](Cc2ccccc2)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 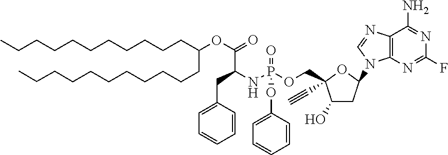 [SMILES: C#C[C@]1(CO[P@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 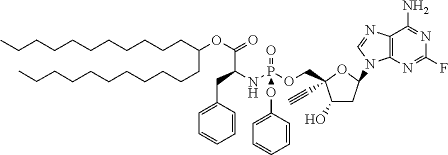 [SMILES: C#C[C@]1(CO[P@@](=O)(N[C@@H](Cc2ccccc2)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 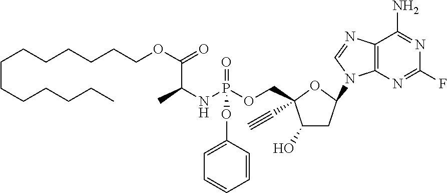 [SMILES: C#C[C@]1(CO[P@](=O)(N[C@@H](C)C(=O)OCCCCCCCCCCCCC)Oc2ccccc2)O[C@@H](n2cnc3c(N)nc(F)nc32)C[C@@H]1O] | Tridecyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 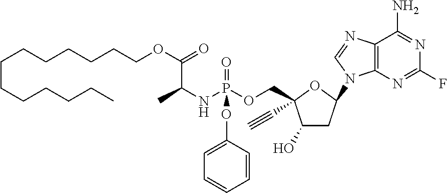 [SMILES: C#C[C@]1(CO[P@@](=O)(N[C@@H](C)C(=O)OCCCCCCCCCCCCC)Oc2ccccc2)O[C@@H](n2cnc3c(N)nc(F)nc32)C[C@@H]1O] | Tridecyl ((S)-(((2R,3S,5R)-5-(6-amino-2- fluoro-9H-purin-9-yl)-2-ethynyl- 3-hydroxytetrahydrofuran-2-yl) methoxy)(phenoxy)phosphoryl)- L-alaninate |
| 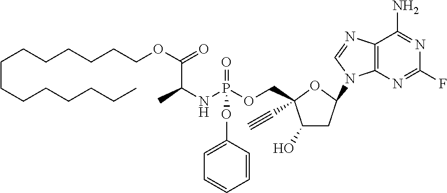 [SMILES: C#C[C@]1(CO[P@](=O)(N[C@@H](C)C(=O)OCCCCCCCCCCCCCC)Oc2ccccc2)O[C@@H](n2cnc3c(N)nc(F)nc32)C[C@@H]1O] | Tetradecyl ((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 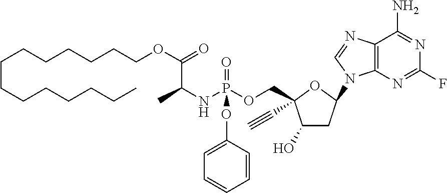 [SMILES: C#C[C@]1(CO[P@@](=O)(N[C@@H](C)C(=O)OCCCCCCCCCCCCCC)Oc2ccccc2)O[C@@H](n2cnc3c(N)nc(F)nc32)C[C@@H]1O] | Tetradecyl ((S)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 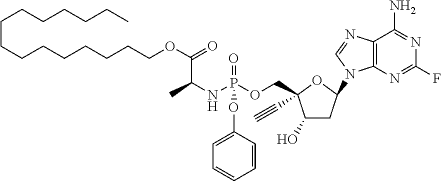 [SMILES: C#C[C@]1(CO[P@](=O)(N[C@@H](C)C(=O)OCCCCCCCCCCCCCCC)Oc2ccccc2)O[C@@H](n2cnc3c(N)nc(F)nc32)C[C@@H]1O] | Pentadecyl ((R)-(((2R,3S,5R)-5-(6-amino- 2-fluoro-9H-purin-9-yl)-2-ethynyl-3- hydroxytetrahydrofuran-2yl)- methoxy)(phenoxy)phosphoryl)- L-alaninate |
| 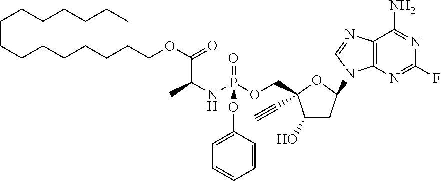 [SMILES: C#C[C@]1(CO[P@@](=O)(N[C@@H](C)C(=O)OCCCCCCCCCCCCCCC)Oc2ccccc2)O[C@@H](n2cnc3c(N)nc(F)nc32)C[C@@H]1O] | Pentadecyl ((S)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 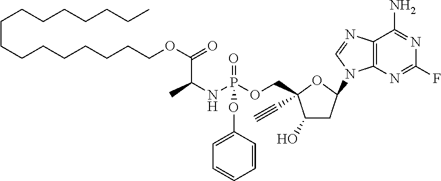 [SMILES: C#C[C@]1(CO[P@](=O)(N[C@@H](C)C(=O)OCCCCCCCCCCCCCCCC)Oc2ccccc2)O[C@@H](n2cnc3c(N)nc(F)nc32)C[C@@H]1O] | Hexadecyl ((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 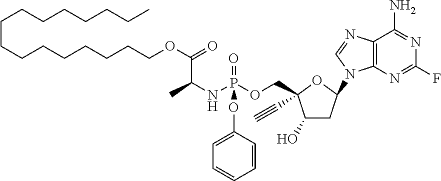 [SMILES: C#C[C@]1(CO[P@@](=O)(N[C@@H](C)C(=O)OCCCCCCCCCCCCCCCC)Oc2ccccc2)O[C@@H](n2cnc3c(N)nc(F)nc32)C[C@@H]1O] | Hexadecyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 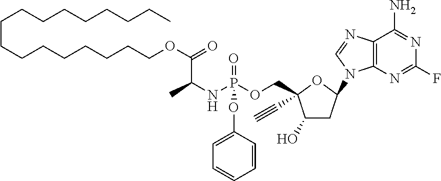 [SMILES: C#C[C@]1(CO[P@](=O)(N[C@@H](C)C(=O)OCCCCCCCCCCCCCCCCC)Oc2ccccc2)O[C@@H](n2cnc3c(N)nc(F)nc32)C[C@@H]1O] | Heptadecyl ((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 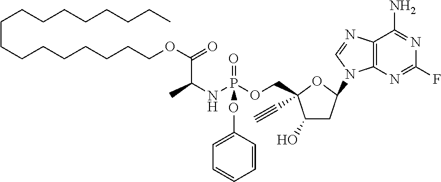 [SMILES: C#C[C@]1(CO[P@@](=O)(N[C@@H](C)C(=O)OCCCCCCCCCCCCCCCCC)Oc2ccccc2)O[C@@H](n2cnc3c(N)nc(F)nc32)C[C@@H]1O] | Heptadecyl ((S)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 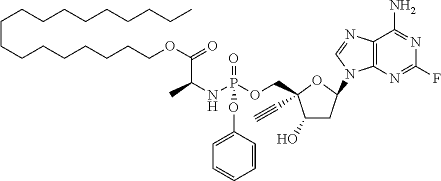 [SMILES: C#C[C@]1(CO[P@](=O)(N[C@@H](C)C(=O)OCCCCCCCCCCCCCCCCCC)Oc2ccccc2)O[C@@H](n2cnc3c(N)nc(F)nc32)C[C@@H]1O] | Octadecyl ((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 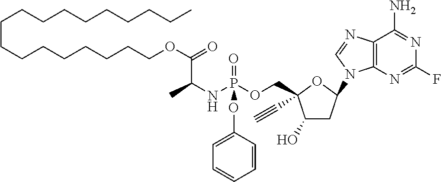 [SMILES: C#C[C@]1(CO[P@@](=O)(N[C@@H](C)C(=O)OCCCCCCCCCCCCCCCCCC)Oc2ccccc2)O[C@@H](n2cnc3c(N)nc(F)nc32)C[C@@H]1O] | Octadecyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 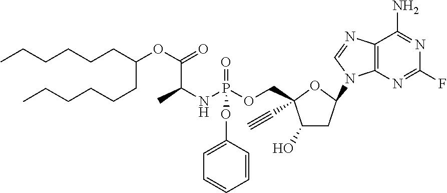 [SMILES: C#C[C@]1(CO[P@](=O)(N[C@@H](C)C(=O)OC(CCCCCC)CCCCCC)Oc2ccccc2)O[C@@H](n2cnc3c(N)nc(F)nc32)C[C@@H]1O] | Tridecan-7-yl ((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 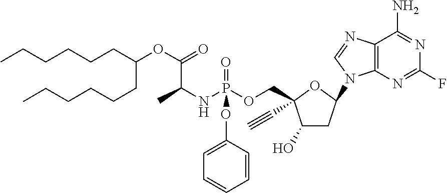 [SMILES: C#C[C@]1(CO[P@@](=O)(N[C@@H](C)C(=O)OC(CCCCCC)CCCCCC)Oc2ccccc2)O[C@@H](n2cnc3c(N)nc(F)nc32)C[C@@H]1O] | Tridecan-7-yl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-alaninate |
| 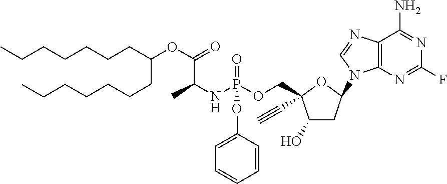 [SMILES: C#C[C@]1(CO[P@](=O)(N[C@@H](C)C(=O)OC(CCCCCCC)CCCCCCC)Oc2ccccc2)O[C@@H](n2cnc3c(N)nc(F)nc32)C[C@@H]1O] | Pentadecan-8-yl ((R)-(((2R,3S,5R)-5-(6- amino-2-fluoro-9H-purin-9-yl)-2-ethynyl- 3-hydroxytetrahydrofuran-2- yl)methoxy)(phenoxy)phosphoryl)- L-alaninate |
| 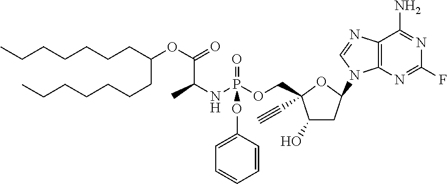 [SMILES: C#C[C@]1(CO[P@@](=O)(N[C@@H](C)C(=O)OC(CCCCCCC)CCCCCCC)Oc2ccccc2)O[C@@H](n2cnc3c(N)nc(F)nc32)C[C@@H]1O] | Pentadecan-8-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 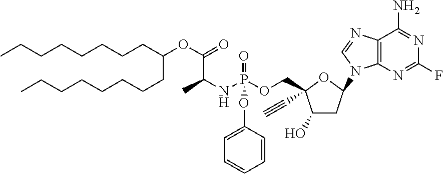 [SMILES: C#C[C@]1(CO[P@](=O)(N[C@@H](C)C(=O)OC(CCCCCCCC)CCCCCCCC)Oc2ccccc2)O[C@@H](n2cnc3c(N)nc(F)nc32)C[C@@H]1O] | Heptadecan-9-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 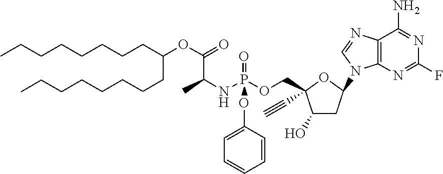 [SMILES: C#C[C@]1(CO[P@@](=O)(N[C@@H](C)C(=O)OC(CCCCCCCC)CCCCCCCC)Oc2ccccc2)O[C@@H](n2cnc3c(N)nc(F)nc32)C[C@@H]1O] | heptadecan-9-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 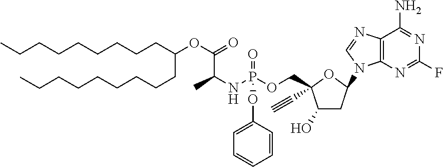 [SMILES: C#C[C@]1(CO[P@](=O)(N[C@@H](C)C(=O)OC(CCCCCCCCC)CCCCCCCCC)Oc2ccccc2)O[C@@H](n2cnc3c(N)nc(F)nc32)C[C@@H]1O] | Nonadecan-10-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 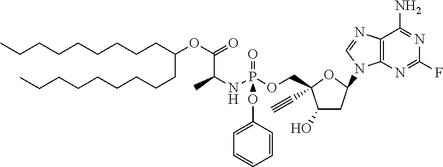 [SMILES: C#C[C@]1(CO[P@@](=O)(N[C@@H](C)C(=O)OC(CCCCCCCCC)CCCCCCCCC)Oc2ccccc2)O[C@@H](n2cnc3c(N)nc(F)nc32)C[C@@H]1O] | Nonadecan-10-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 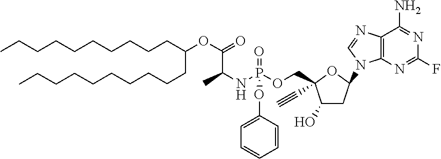 [SMILES: C#C[C@]1(CO[P@](=O)(N[C@@H](C)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 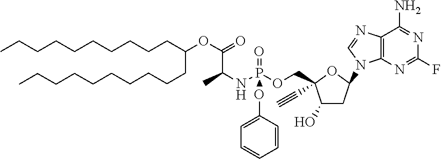 [SMILES: C#C[C@]1(CO[P@@](=O)(N[C@@H](C)C(=O)OC(CCCCCCCCCC)CCCCCCCCCC)Oc2ccccc2)O[C@@H](n2cnc3c(N)nc(F)nc32)C[C@@H]1O] | Henicosan-11-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 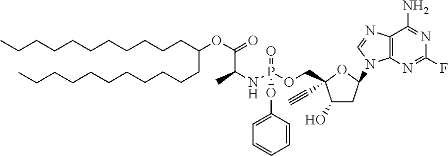 [SMILES: C#C[C@]1(CO[P@](=O)(N[C@@H](C)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)- L-alaninate |
| 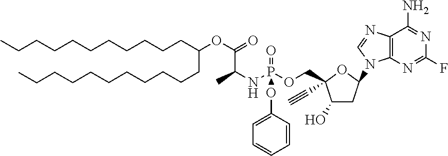 [SMILES: C#C[C@]1(CO[P@@](=O)(N[C@@H](C)C(=O)OC(CCCCCCCCCCC)CCCCCCCCCCC)Oc2ccccc2)O[C@@H](n2cnc3c(N)nc(F)nc32)C[C@@H]1O] | Tricosan-12-yl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-alaninate |
| 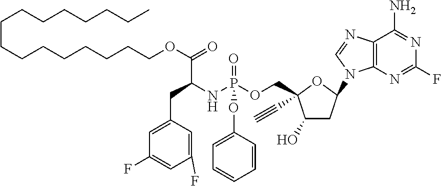 [SMILES: C#C[C@]1(CO[P@](=O)(N[C@@H](Cc2cc(F)cc(F)c2)C(=O)OCCCCCCCCCCCCCCCC)Oc2ccccc2)O[C@@H](n2cnc3c(N)nc(F)nc32)C[C@@H]1O] | Hexadecyl (S)-2-(((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 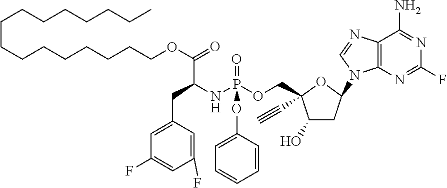 [SMILES: C#C[C@]1(CO[P@@](=O)(N[C@@H](Cc2cc(F)cc(F)c2)C(=O)OCCCCCCCCCCCCCCCC)Oc2ccccc2)O[C@@H](n2cnc3c(N)nc(F)nc32)C[C@@H]1O] | Hexadecyl (S)-2-(((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 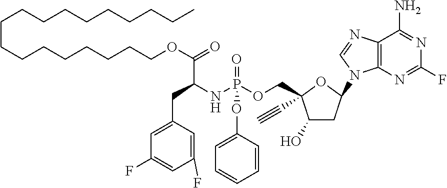 [SMILES: C#C[C@]1(CO[P@](=O)(N[C@@H](Cc2cc(F)cc(F)c2)C(=O)OCCCCCCCCCCCCCCCCCC)Oc2ccccc2)O[C@@H](n2cnc3c(N)nc(F)nc32)C[C@@H]1O] | Octadecyl (S)-2-(((R)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 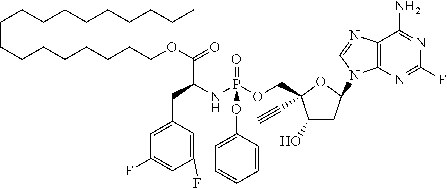 [SMILES: C#C[C@]1(CO[P@@](=O)(N[C@@H](Cc2cc(F)cc(F)c2)C(=O)OCCCCCCCCCCCCCCCCCC)Oc2ccccc2)O[C@@H](n2cnc3c(N)nc(F)nc32)C[C@@H]1O] | octadecyl (S)-2-(((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 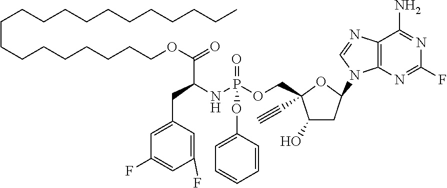 [SMILES: C#C[C@]1(CO[P@](=O)(N[C@@H](Cc2cc(F)cc(F)c2)C(=O)OCCCCCCCCCCCCCCCCCCCC)Oc2ccccc2)O[C@@H](n2cnc3c(N)nc(F)nc32)C[C@@H]1O] | Icosyl (S)-2-(((R)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 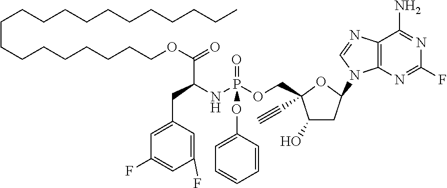 [SMILES: C#C[C@]1(CO[P@@](=O)(N[C@@H](Cc2cc(F)cc(F)c2)C(=O)OCCCCCCCCCCCCCCCCCCCC)Oc2ccccc2)O[C@@H](n2cnc3c(N)nc(F)nc32)C[C@@H]1O] | Icosyl (S)-2-(((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 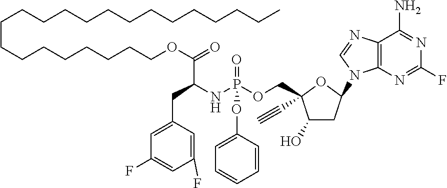 [SMILES: C#C[C@]1(CO[P@](=O)(N[C@@H](Cc2cc(F)cc(F)c2)C(=O)OCCCCCCCCCCCCCCCCCCCCCC)Oc2ccccc2)O[C@@H](n2cnc3c(N)nc(F)nc32)C[C@@H]1O] | Docosyl (S)-2-(((R)-(((2R,3S,5R)-5-(6-amino- 2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 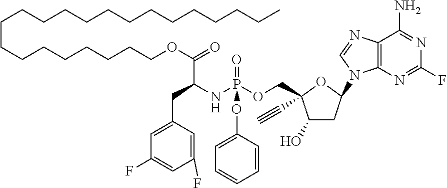 [SMILES: C#C[C@]1(CO[P@@](=O)(N[C@@H](Cc2cc(F)cc(F)c2)C(=O)OCCCCCCCCCCCCCCCCCCCCCC)Oc2ccccc2)O[C@@H](n2cnc3c(N)nc(F)nc32)C[C@@H]1O] | Docosyl (S)-2-(((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)amino)- 3-(3,5-difluorophenyl)propanoate |
| 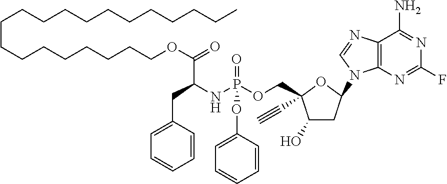 [SMILES: C#C[C@]1(CO[P@](=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1O] | Icosyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 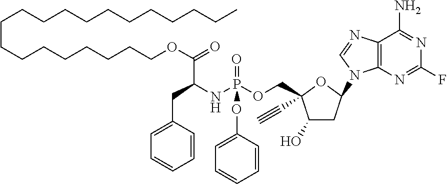 [SMILES: C#C[C@]1(CO[P@@](=O)(N[C@@H](Cc2ccccc2)C(=O)OCCCCCCCCCCCCCCCCCCCC)Oc2ccccc2)O[C@@H](n2cnc3c(N)nc(F)nc32)C[C@@H]1O] | Icosyl ((S)-(((2R,3S,5R)-5-(6-amino-2-fluoro- 9H-purin-9-yl)-2-ethynyl-3- hydroxytetrahydrofuran-2-yl) methoxy)(phenoxy)phosphoryl)- L-phenylalaninate |
| 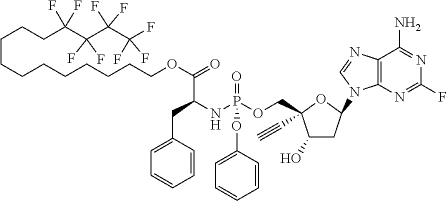 [SMILES: C#C[C@]1(CO[P@](=O)(N[C@@H](Cc2ccccc2)C(=O)OCCCCCCCCCCCC(F)(F)C(F)(F)C(F)(F)C(F)(F)F)Oc2ccccc2)O[C@@H](n2cnc3c(N)nc(F)nc32)C[C@@H]1O] | 12,12,13,13,14,14,15,15,15- Nonafluoropentadecyl ((R)-(((2R,3S,5R)-5- (6-amino-2-fluoro-9H-purin-9-yl)-2-ethynyl- 3-hydroxytetrahydrofuran-2-yl) methoxy)(phenoxy)phosphoryl)- L-phenylalaninate |
| 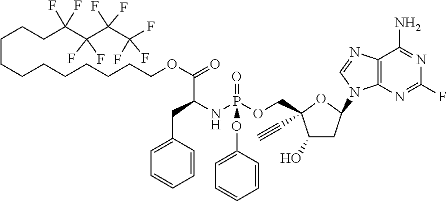 [SMILES: C#C[C@]1(CO[P@@](=O)(N[C@@H](Cc2ccccc2)C(=O)OCCCCCCCCCCCC(F)(F)C(F)(F)C(F)(F)C(F)(F)F)Oc2ccccc2)O[C@@H](n2cnc3c(N)nc(F)nc32)C[C@@H]1O] | 12,12,13,13,14,14,15,15,15- Nonafluoropentadecyl ((S)-(((2R,3S,5R)-5- (6-amino-2-fluoro-9H-purin-9-yl)-2-ethynyl- 3-hydroxytetrahydrofuran-2-yl) methoxy)(phenoxy)phosphoryl)- L-phenylalaninate |
| 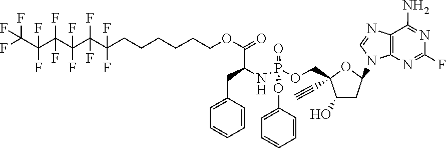 | 7,7,8,8,9,9,10,10,11,11,12,12,12- Tridecafluorododecyl ((R)-(((2R,3S,5R)-5- (6-amino-2-fluoro-9H-purin-9-yl)-2-ethynyl- 3-hydroxytetrahydrofuran-2-yl) methoxy)(phenoxy)phosphoryl)- L-phenylalaninate |
| 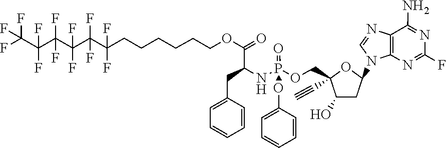 | 7,7,8,8,9,9,10,10,11,11,12,12,12- Tridecafluorododecyl ((S)-(((2R,3S,5R)-5-(6- amino-2-fluoro-9H-purin-9-yl)-2-ethynyl- 3-hydroxytetrahydrofuran-2-yl) methoxy)(phenoxy)phosphoryl)- L-phenylalaninate |
| 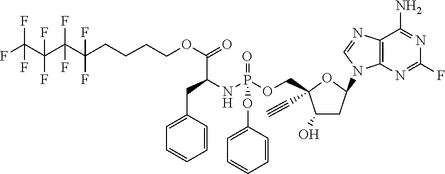 | 5,5,6,6,7,7,8,8,8-Nonafluorooctyl ((R)- (((2R,3S,5R)-5-(6-amino-2-fluoro-9H-purin- 9-yl)-2-ethynyl-3-hydroxytetrahydrofuran-2- yl)methoxy)(phenoxy)phosphoryl)- L-phenylalaninate |
| 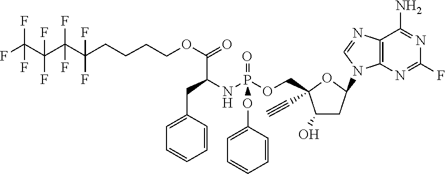 | 5,5,6,6,7,7,8,8,8-Nonafluorooctyl ((S)- (((2R,3S,5R)-5-(6-amino-2-fluoro-9H-purin- 9-yl)-2-ethynyl-3-hydroxytetrahydrofuran-2- yl)methoxy)(phenoxy)phosphoryl)- L-phenylalaninate |
| 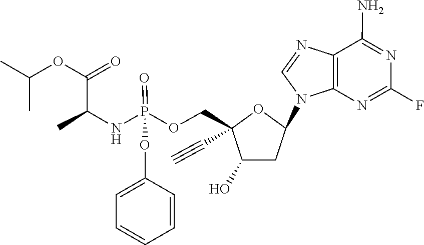 | Isopropyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 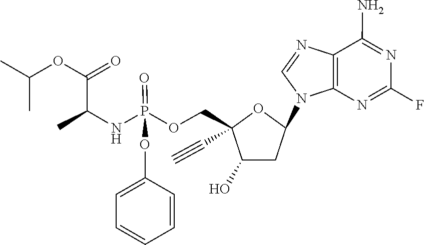 | Isopropyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 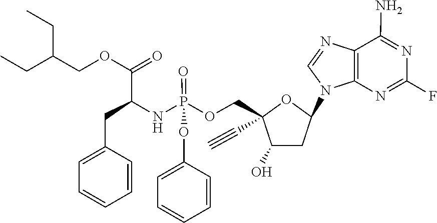 | 2-Ethylbutyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 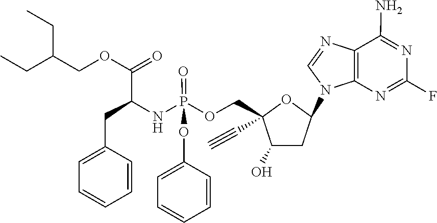 | 2-Ethylbutyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 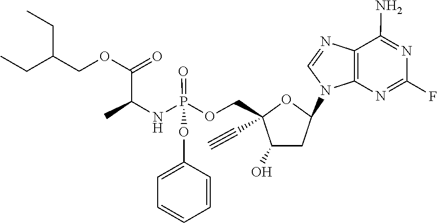 | 2-Ethylbutyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 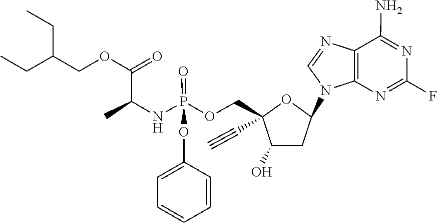 | 2-Ethylbutyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-alaninate |
| 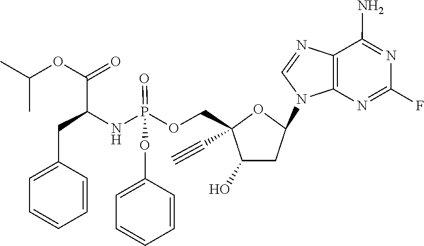 | Isopropyl ((R)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 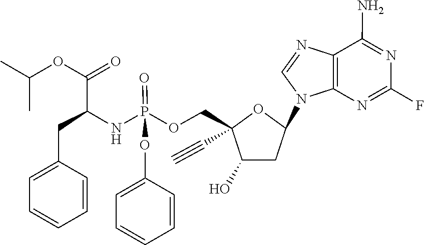 | Isopropyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 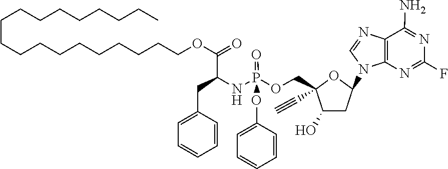 | Nonadecyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 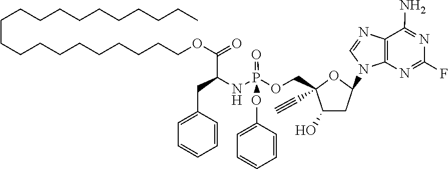 | Henicosyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 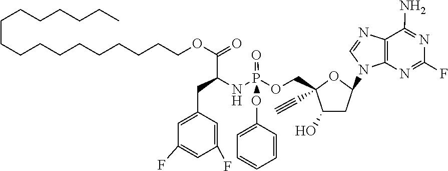 | Pentadecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 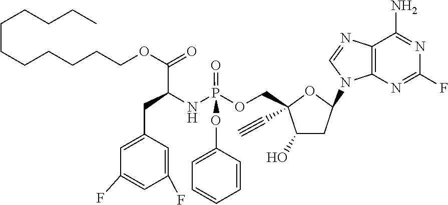 | Undecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 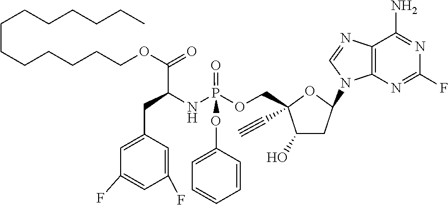 | Tridecyl (S)-2-(((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 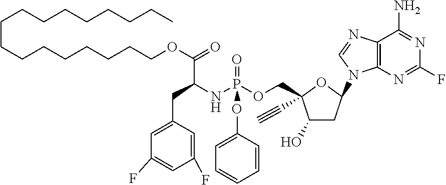 | Heptadecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 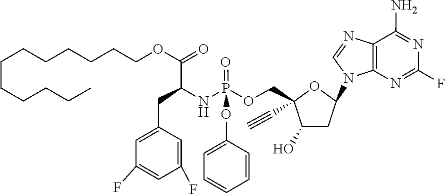 | Dodecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 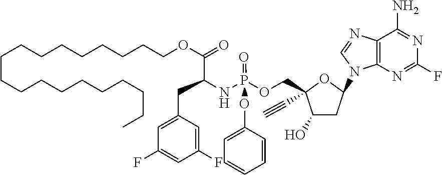 | Nonadecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 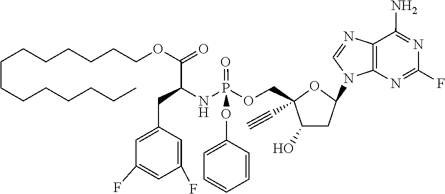 | Tetradecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 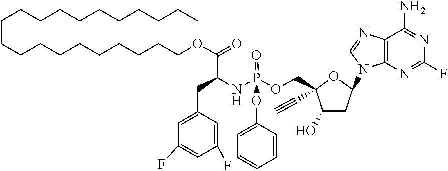 | Henicosyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 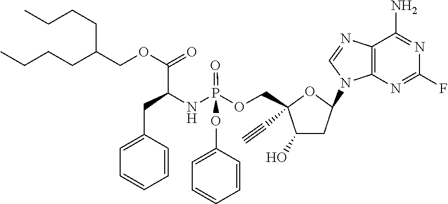 | 2-Butylhexyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 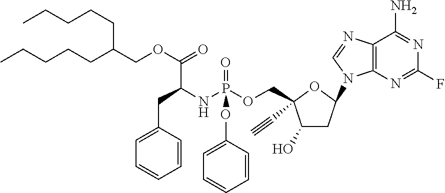 | 2-Pentylheptyl ((S)-(((2R,3S,5R)-5-(6-amino- 2-fluoro-9H-purin-9-yl)-2-ethynyl-3- hydroxytetrahydrofuran-2-yl) methoxy)(phenoxy)phosphoryl)- L-phenylalaninate |
| 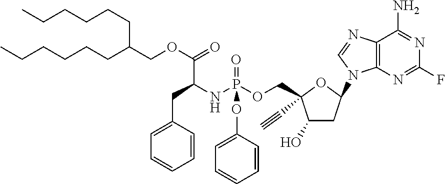 | 2-Hexyloctyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 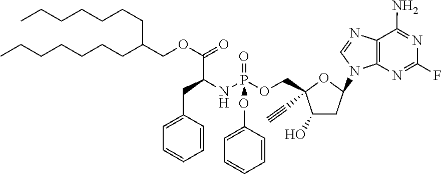 | 2-Heptylnonyl ((((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 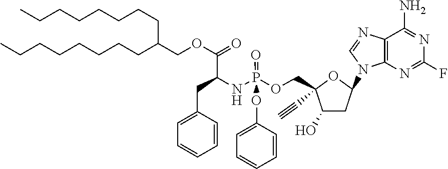 | 2-Octyldecyl ((S)-(((2R,3S,5R)-5-(6-amino-2- fluoro-9H-purin-9-yl)-2-ethynyl-3- hydroxytetrahydrofuran-2-yl) methoxy)(phenoxy)phosphoryl)- L-phenylalaninate |
| 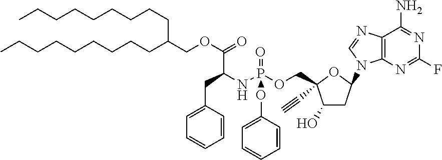 | 2-Nonylundecyl ((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)- L-phenylalaninate |
| 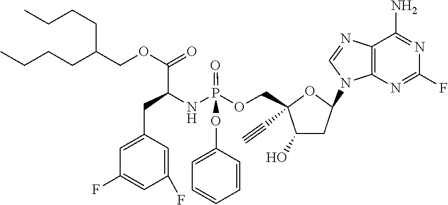 | 2-Butylhexyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 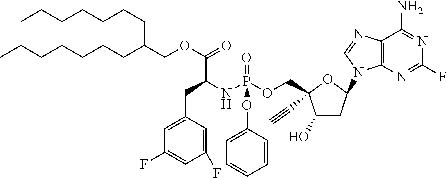 | 2-Heptylnonyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 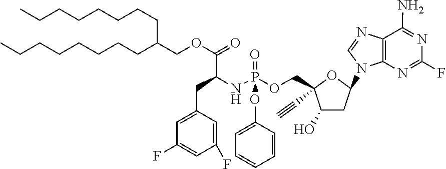 | 2-Octyldecyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 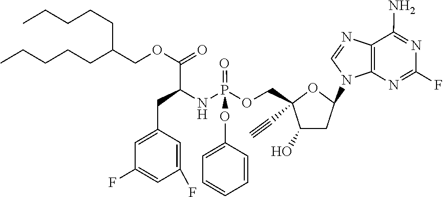 | 2-Pentylheptyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)- 3-(3,5-difluorophenyl)propanoate |
| 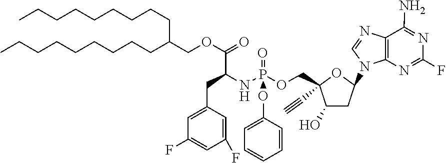 | 2-Nonylundecyl (S)-2-(((S)-(((2R,3S,5R)-5- (6-amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |
| 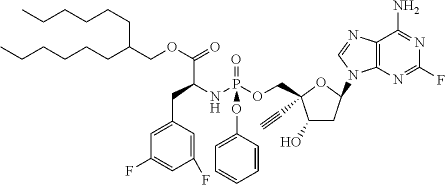 | 2-Hexyloctyl (S)-2-(((S)-(((2R,3S,5R)-5-(6- amino-2-fluoro-9H-purin-9-yl)-2-ethynyl-3- hydroxytetrahydrofuran-2-yl) methoxy)(phenoxy)phosphoryl)amino)-3- (3,5-difluorophenyl)propanoate |

pharmaceutically acceptable salts thereof.

14. A compound of formula (I): 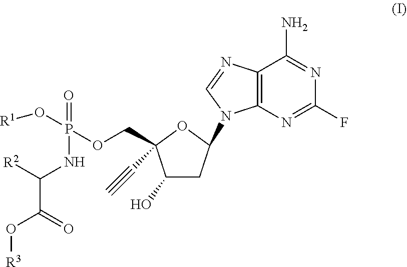
wherein:
R¹ is aryl;
R² is selected from the group consisting of (C₁-C₁₀) alkylaryl;
R³ is aryl; and
R⁴ is selected from the group consisting of (C₁-C₂₅) alkyl; and
wherein each of R¹, R², R³, and R⁴ may be independently and optionally substituted by one or more Cl or F;
or pharmaceutically acceptable salts thereof.

15. The compound of claim 14, or a pharmaceutically acceptable salt thereof, wherein R² is (C₁-C₄) alkylaryl.

16. The compound of claim 14, or a pharmaceutically acceptable salt thereof, wherein R² is (C₁)alkylaryl.

17. The compound of claim 14, or a pharmaceutically acceptable salt thereof, wherein at least one of R² and R³ is substituted by one or more of Cl or F.

18. The compound of claim 14, or a pharmaceutically acceptable salt thereof, wherein at least one of R² and R³ is substituted by one or more F.

19. The compound of claim 14, or a pharmaceutically acceptable salt thereof, wherein R² is (C₁) alkyl-aryl, wherein C₆ aryl is substituted by two substituents which are F.

20. The compound of claim 14, or a pharmaceutically acceptable salt thereof, wherein R² is:

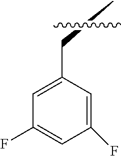

21. The compound of claim 14, or a pharmaceutically acceptable salt thereof, wherein R³ is (C₅-C₂₀) alkyl

22. The compound of claim 14, or a pharmaceutically acceptable salt thereof, wherein R³ is (C₅-C₂₀) alkyl substituted by two or more substituents which are each F.

23. The compound of claim 14, or a pharmaceutically acceptable salt thereof, wherein R³ is (C₅-C₁₅) alkyl, substituted by 5 to 15 substituents which are each F.

24. A pharmaceutical composition comprising a compound according to claim 1, or a pharmaceutically acceptable salt thereof, and a pharmaceutically acceptable excipient.

25. The composition of claim 24, wherein the composition is present in parenteral form.

26. The composition of claim 24, wherein the composition is in a tablet form.

27. The composition of claim 24, wherein the composition is formulated as a long acting parenteral injection.

28. A method of treating an HIV infection in a patient comprising administering to the subject a compound of claim 1, or a pharmaceutically acceptable salt thereof.

29. A method of preventing an HIV infection in a patient at risk for developing an HIV infection, comprising administering to the subject a compound of claim 1, or a pharmaceutically acceptable salt thereof.

30. (canceled)

31. (canceled)

32. (canceled)

33. (canceled)

34. A combination comprising a compound according claim 1, or a pharmaceutically acceptable salt thereof, and one or more additional agents active against HIV.

35. A pharmaceutical composition comprising a combination according to claim 34 and a pharmaceutically acceptable excipient.

36. A method of treating an HIV infection in a patient comprising administering to the subject a combination of claim 34.

37. A method of preventing an HIV infection in a patient at risk for developing an HIV infection, comprising administering to the subject a combination of claim 34.

38. (canceled)

39. (canceled)

40. (canceled)

41. (canceled)
